(19) United States
(12) Patent Application Publication
(10) Pub. No.: US 2025/0136619 A1
(43) Pub. Date: May 1, 2025

(54) HETEROBIFUNCTIONAL COMPOUNDS AND THEIR USE IN TREATING DISEASE

(71) Applicant: HALDA THERAPEUTICS OPCO, INC., New Haven, CT (US)

(72) Inventors: Samuel W. GERRITZ, Guilford, CT (US); Katherine J. KAYSER-BRICKER, Branford, CT (US); Kyle J. EASTMAN, Killingworth, CT (US); Taavi NEKLESA, Orange, CT (US); Kanak Shail RAINA, Branford, CT (US); James John MOUSSEAU, Salem, CT (US); Nilesh ZAWARE, Hamden, CT (US)

(21) Appl. No.: 18/696,622

(22) PCT Filed: Oct. 4, 2022
(86) PCT No.: PCT/US2022/045603

§ 371 Date: Mar. 28, 2024

Related U.S. Application Data
(60) Provisional application No. 63/251,713, filed on Oct. 4, 2021.

(51) Int. Cl. C07D 519/00; A61K 31/4439; A61K 31/4709; A61K 31/496; A61K 31/506; A61K 31/519; A61K 31/5365; A61K 31/5377; A61K 31/551; A61P 35/00; C07D 239/48; C07D 401/14; C07D 403/12; C07D 403/14; C07D 417/14; C07D 471/04; C07D 487/04; C07D 495/14; C07D 498/04

(52) U.S. Cl. CPC C07D 519/00; A61K 31/4439; A61K 31/4709; A61K 31/496; A61K 31/506; A61K 31/519; A61K 31/5365; A61K 31/5377; A61K 31/551; A61P 35/00; C07D 239/48; C07D 401/14; C07D 403/12; C07D 403/14; C07D 417/14; C07D 471/04; C07D 487/04; C07D 495/14; C07D 498/04

(57) ABSTRACT

The invention provides heterobifunctional compounds comprising an effector protein binding moiety selected from mTor, PLK1, CDKI, CDK2, CDK9, BRD4, AURKA, AURKB, MEK, Src, c-KIT, KIF11, HSP90, tubulin, proteasome, topoisomerase or HD AC which is linked to a moiety that binds to a target protein selected from KRAS, HER2 or EGFR. Pharmaceutical compositions and their use in treating disease, such as cancer, are also disclosed.

### CROSS-REFERENCE TO RELATED APPLICATION

This application claims priority to U.S. provisional patent application No. 63/251,713, filed on Oct. 4, 2021, the contents of which are hereby incorporated by reference in their entirety.

### FIELD OF THE INVENTION

The invention provides heterobifunctional compounds, pharmaceutical compositions, and their use in treating disease, such as cancer.

### BACKGROUND

Cancer continues to be a significant health problem despite the substantial research efforts and scientific advances reported in the literature for treating this disease. Solid tumors, including prostate cancer, breast cancer, and lung cancer remain highly prevalent among the world population. The incidence of prostate cancer increases with age, and with increasing longevity of human subjects, there continues to be a corresponding rise in the number of patients suffering from prostate cancer. Breast cancer is one of the most common cancers among women and is a leading cause of death for women between ages 50-55. Lung cancer is a leading cause of death among cancer patients, where over 85% of lung cancers are non-small cell lung cancer (NSCLC). Many lung cancers are attributed to tobacco smoking. Current treatment options for these cancers are not effective for all patients and/or can have substantial adverse side effects.

New therapies are needed to address this unmet need in cancer therapy. In particular, new therapies are needed that achieve an anti-cancer effect through a different mechanism than commonly available therapies. Exemplary mechanisms for common anti-cancer therapies include (a) alkylation of DNA which limits ability of the cell to reproduce, (b) topoisomerase inhibition, in which the therapeutic agent inhibits the activity of a topoisomerases thereby limiting separation of strands of DNA, and (c) mitotic inhibition, where the therapeutic agent reduces ability of the cell to divide. New therapies that achieve an anti-cancer effect through a different mechanism present an opportunity to treat cancers more effectively and/or to treat cancers that have become resistant to currently available medicines.

The present invention addresses the foregoing needs and provides other related advantages.

### SUMMARY

The invention provides heterobifunctional compounds, pharmaceutical compositions, and their use in treating disease, such as cancer. In particular, one aspect of the invention provides a collection of heterobifunctional compounds, such as a compound represented by Formula I:

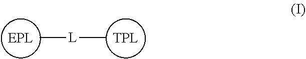

or a pharmaceutically acceptable salt thereof, where the variables are as defined in the detailed description. Further description of additional collections of heterobifunctional compounds are described in the detailed description. The compounds may be part of a pharmaceutical composition comprising a pharmaceutically acceptable carrier.

Another aspect of the invention provides a method of treating cancer. The method comprises administering to a patient in need thereof a therapeutically effective amount of a compound described herein, such as a compound of Formula I to treat the cancer.

Another aspect of the invention provides a method of treating hepatitis. The method comprises administering to a patient in need thereof a therapeutically effective amount of a compound described herein, such as a compound of Formula I, to treat the hepatitis.

Another aspect of the invention provides a method of causing death of a cancer cell. The method comprises contacting a cancer cell with an effective amount of a compound described herein, such as a compound of Formula I, to cause death of the cancer cell.

### DETAILED DESCRIPTION

The invention provides heterobifunctional compounds, pharmaceutical compositions, and their use in treating disease, such as cancer. The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C.C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

Compounds of the present invention include those described generally herein, and are further illustrated by the classes, subclasses, and species disclosed herein. As used herein, the following definitions shall apply unless otherwise indicated. These definitions apply regardless of whether a term is used by itself or in combination with other terms, unless otherwise indicated. Hence, the definition of “alkyl” applies to “alkyl” as well as the “alkyl” portions of “—O-alkyl” etc. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl) alkyl, (cycloalkenyl) alkyl or (cycloalkyl) alkenyl.

As used herein, the term “bicyclic ring” or “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

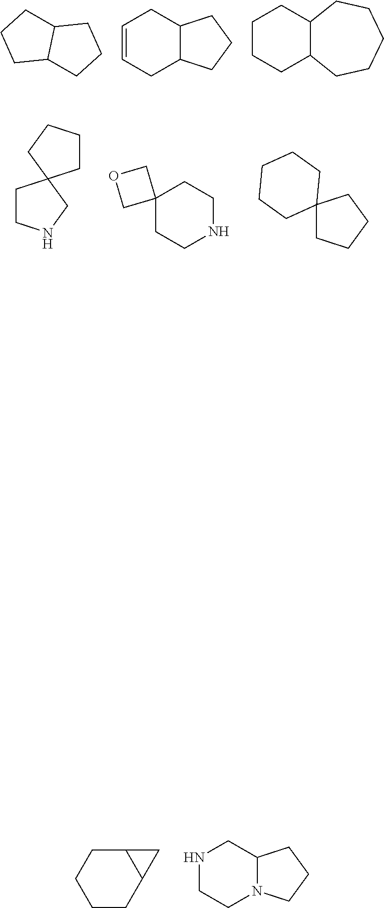

Exemplary bridged bicyclics include:

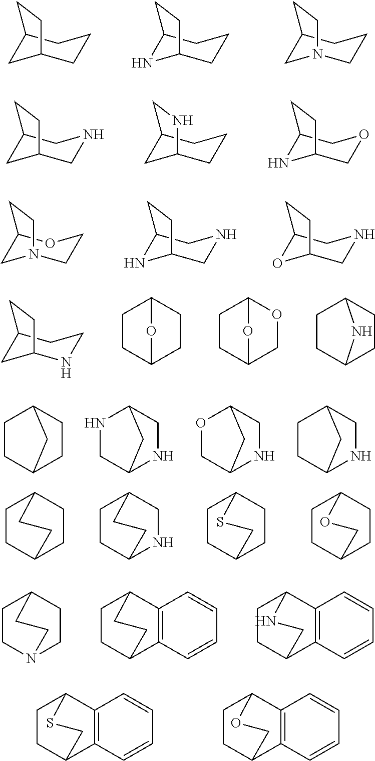

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N(as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “—(C₀ alkylene)-” refers to a bond. Accordingly, the term “—(C₀₋₃ alkylene)-” encompasses a bond (i.e., C₀) and a —(C₁₋₃ alkylene)-group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “haloaryl” refers to an aryl group that is substituted with at least one halogen. Exemplary haloaryl groups include chlorophenyl (e.g., 3-chlorophenyl, 4-chlorophenyl), fluorophenyl, and the like. The term “phenylene” refers to a bivalent phenyl group.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused.

Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted. The term “haloheteroaryl” refers to a heteroaryl group that is substituted with at least one halogen. Exemplary haloheteroaryl groups include chloropyridine, fluoropyridine, chloropyrazole, fluoropyrazole, and the like. The term “heteroarylene” refers to a bivalent heteroaryl group. Similarly, the terms “pyrazolylene”, “imidazolylene”, and “pyrrolylene”, respectively refer to bivalent pyrazolyl, imidazolyl, and pyrrolyl groups. Similarly, the terms “pyridinylene” and “pyrimidinylene”, respectively refer to bivalent pyridinyl and pyrimidinyl groups.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N(as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted. The term “heterocyclylene” refers to a bivalent heterocyclyl group.

As used herein, the term “heterocycloakyl” refers to a saturated heterocyclyl. The term “heterocycloakyl” refers to a bivalent heterocycloakyl group.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{o}; —(CH₂)₀₋₄OR^{o}; —O(CH₂)₀₋₄R^{o}, —O—(CH₂)₀₋₄ C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR₂^{o}; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR—, SC(S)SR^{o}; —(CH₂)₀₋₄ SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄ S(O)₂R^{o}; —(CH₂)₀₋₄S(O)₂OR^{o}; —(CH₂)₀₋₄OS(O)₂R^{o}; —S(O)₂NR₂^{o}; —S(O)(NR^{o})R^{o}; —S(O)₂N═C(NR^{o}₂)₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR₂^{o}; —P(O)₂R^{o}; —P(O)R^{o}2; —OP(O)R^{o}2; —OP(O)(OR^{o})₂; SiR₃^{o}; —(C₁₋₄ straight or branched alkylene)O—N(R^{o})₂; or —(C₁₋₄ straight or branched alkylene)C(O)O—N(R^{o})₂.

Each R^{o} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{o}selected from ═O and ═S; or each R^{o} is optionally substituted with a monovalent substituent independently selected from halogen, —(CH₂)₀₋₂R•, -(haloR•), —(CH₂)₀₋₂OH, —(CH₂)₀₋₂OR•, —(CH₂)₀₋₂CH(OR•)₂; —O(haloR•), —CN, —N₃, —(CH₂)₀₋₂C(O)R•, —(CH₂)₀₋₂C(O)OH, —(CH₂)₀₋₂ C(O)OR•, —(CH₂)₀₋₂SR•, —(CH₂)₀₋₂SH, —(CH₂)₀₋₂NH₂, —(CH₂)₀₋₂NHR•, —(CH₂)₀₋₂NR•₂, —NO₂, —SiR•₃, —OSiR•₃, —C(O)SR•, —(C₁₋₄ straight or branched alkylene)C(O)OR•, or —SSR•.

Each R• is independently selected from C₁_₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R• is unsubstituted or where preceded by halo is substituted only with one or more halogens; or wherein an optional substituent on a saturated carbon is a divalent substituent independently selected from ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R•, -(haloR•), —OH, —OR•, —O(haloR•), —CN, —C(O)OH, —C(O)OR•, —NH₂, —NHR•, —NR•₂, or —NO₂, wherein each R• is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R• is unsubstituted or where preceded by halo is substituted only with one or more halogens.

An optional substituent on a substitutable nitrogen is independently —R^{†}, —NR₂^{†}, —C(O)R^{†}, —C(O)OR^{†}, —C(O)C(O)R^{†}, —C(O)CH₂C(O)R^{†}, —S(O)₂R^{†}, —S(O)₂NR₂^{†}, —C(S)NR₂^{†}, —C(NH)NR₂^{†}, or —N(R^{†})S(O)₂R^{†}; wherein each R^{†} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ aliphatic, R^{†} is optionally substituted with halogen, —R^{•}, -(haloR•), —OH, —OR•, —O(haloR•), —CN, —C(O)OH, —C(O)OR•, —NH₂, —NHR•, —NR₂•, or —NO₂, wherein each R• is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R• is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977)66 (1)1-19; P. Gould, International J. of Pharmaceutics (1986)33 201-217; Anderson et al., The Practice of Medicinal Chemistry (1996), Academic Press, New York; and in The Orange Book (Food & Drug Administration, Washington, D.C. on their website). These disclosures are incorporated herein by reference.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as a atropisomer (e.g., substituted biaryls), all forms of such atropisomer are considered part of this invention.

Chemical names, common names, and chemical structures may be used interchangeably to describe the same structure. If a chemical compound is referred to using both a chemical structure and a chemical name, and an ambiguity exists between the structure and the name, the structure predominates. It should also be noted that any carbon as well as heteroatom with unsatisfied valences in the text, schemes, examples and tables herein is assumed to have the sufficient number of hydrogen atom(s) to satisfy the valences.

The terms “a” and “an” as used herein mean “one or more” and include the plural unless the context is inappropriate.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “cycloalkylene” refers to a bivalent cycloalkyl group.

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like. The term “chloroalkyl” refers to an alkyl group that is substituted with at least one chloro. The term “bromoalkyl” refers to an alkyl group that is substituted with at least one bromo. The term “haloalkylene” refers to a bivalent haloalkyl group.

The term “hydroxyalkyl” refers to an alkyl group that is substituted with at least one hydroxyl. Exemplary hydroxyalkyl groups include —CH₂CH₂OH, —C(H)(OH)CH₃, —CH₂C(H)(OH)CH₂CH₂OH, and the like.

The term “heteroalkyl” refers to an alkyl group in which one or more carbon atoms has been replaced by a heteroatom (e.g., N, O, or S). Exemplary heteroalkyl groups include —OCH₃, —CH₂OCH₃, —CH₂CH₂N(CH₃)₂, and —CH₂CH₂OH. The heteroalkyl group may contain, for example, from 2-4, 2-6, or 2-8 atoms selected from the group consisting of carbon and a heteroatom (e.g., N, O, or S). The phrase 3-8 membered heteroalkyl refers to a heteroalkyl group having from 3 to 8 atoms selected from the group consisting of carbon and a heteroatom. The term “heteroalkylene” refers to a bivalent heteroalkyl group.

The terms “alkenyl” and “alkynyl” are art-recognized and refer to unsaturated aliphatic groups analogous in length and possible substitution to the alkyls described above, but that contain at least one double or triple bond respectively. The term “haloalkenyl” refers to an alkenyl group that is substituted with at least one halogen. The term “fluoroalkenyl” refers to an alkenyl group that is substituted with at least one fluoro. The term “nitroalkenyl” refers to an alkenyl group that is substituted with at least one nitro.

The term “carbocyclylene” refers to a bivalent cycloaliphatic group.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. The term “haloalkoxyl” refers to an alkoxyl group that is substituted with at least one halogen. Exemplary haloalkoxyl groups include —OCH₂F, —OCHF₂, —OCF₃, —OCH₂CF₃, —OCF₂CF₃, and the like.

The term “oxo” is art-recognized and refers to a “═O” substituent. For example, a cyclopentane susbsituted with an oxo group is cyclopentanone.

The term “amino” is art-recognized and refers to both unsubstituted and substituted amines, e.g., a moiety that may be represented by the general formulas:

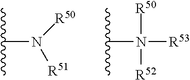

wherein R⁵⁰, R⁵¹, R⁵² and R⁵³ each independently represent a hydrogen, an alkyl, an alkenyl, —(CH₂)ₘ—R⁶¹, or R⁵⁰ and R⁵¹, taken together with the N atom to which they are attached complete a heterocycle having from 4 to 8 atoms in the ring structure; R⁶¹ represents an aryl, a 3-7 membered cycloalkyl, a 4-7 membered cycloalkenyl, 5-10 membered heteroaryl, or 3-10 membered heterocyclyl; and m is zero or an integer in the range of 1 to 8.

The term “amido” is art-recognized and refers to both unsubstituted and substituted amides, e.g., a moiety that may be represented by the general formulas:

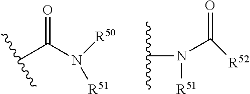

wherein R⁵⁰ and R⁵¹ each independently represent a hydrogen, an alkyl, an alkenyl, —(CH₂) m-R⁶¹, or R⁵⁰ and R⁵¹, taken together with the N atom to which they are attached complete a heterocycle having from 4 to 8 atoms in the ring structure; R⁶¹ represents an aryl, a 3-7 membered cycloalkyl, a 4-7 membered cycloalkenyl, 5-10 membered heteroaryl, or 3-10 membered heterocyclyl; and m is zero or an integer in the range of 1 to 8; and R52 is an alkyl, an alkenyl, or —(CH₂)ₘ—R⁶¹.

The symbol “ [symbol] ” indicates a point of attachment.

When any substituent or variable occurs more than one time in any constituent or the compound of the invention, its definition on each occurrence is independent of its definition at every other occurrence, unless otherwise indicated.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H2O.

As used herein, the terms “subject” and “patient” are used interchangeable and refer to organisms to be treated by the methods of the present invention. Such organisms preferably include, but are not limited to, mammals (e.g., murines, simians, equines, bovines, porcines, canines, felines, and the like), and most preferably includes humans.

The term “IC₅₀” is art-recognized and refers to the concentration of a compound that is required to achieve 50% inhibition of the target.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

In addition, when a compound of the invention contains both a basic moiety (such as, but not limited to, a pyridine or imidazole) and an acidic moiety (such as, but not limited to, a carboxylic acid) zwitterions (“inner salts”) may be formed. Such acidic and basic salts used within the scope of the invention are pharmaceutically acceptable (i.e., non-toxic, physiologically acceptable) salts. Such salts of the compounds of the invention may be formed, for example, by reacting a compound of the invention with an amount of acid or base, such as an equivalent amount, in a medium such as one in which the salt precipitates or in an aqueous medium followed by lyophilization.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### I. Heterobifunctional Compounds

The invention provides heterobifunctional compounds. The compounds are generally represented by the following formula:

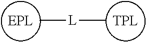

or a pharmaceutically acceptable salt thereof, wherein EPL is a moiety that binds to an effector protein selected from mTOR, PLK1, CDK1, CDK2, CDK9, BRD4, AURKA, AURKB, MEK, Src, c-KIT, KIF11, HSP90, tubulin, proteasome, topoisomerase, or HDAC; L is a linker; and TPL is a moiety that binds to a target protein selected from BTK, androgen receptor protein, or IDH1.

The compounds may be used in the pharmaceutical compositions and therapeutic methods described herein. Exemplary compounds are described in the following sections, along with exemplary procedures for making the compounds.

### Part A: Compounds of Formula I

Another aspect of the invention provides a compound represented by Formula I:

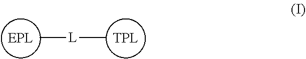

or a pharmaceutically acceptable salt thereof; wherein:
EPL is a moiety that binds to an effector protein selected from mTOR, PLK1, CDK1, CDK2, CDK9, BRD4, AURKA, AURKB, MEK, Src, c-KIT, KIF11, HSP90, tubulin, proteasome, topoisomerase, or HDAC;
L is a linker; and
TPL is a moiety that binds to a target protein selected from BTK, androgen receptor protein, or IDH1.

The definitions of variables in Formula I above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I.

The compound may be further characterized according to, for example, the identity of L and/or TPL. Exemplary further embodiments for L and TPL are provided in Part C below.

As generally defined above, EPL is a moiety that binds to an effector protein selected from mTOR, PLK1, CDK1, CDK2, CDK9, BRD4, AURKA, AURKB, MEK, Src, c-KIT, KIF11, HSP90, Tubulin, Proteasome, Topoisomerase, or HDAC. In certain embodiments, the EPL is a moiety that binds to mTOR, PLK1, CDK1, CDK2, CDK9, or BRD4. In certain embodiments, the EPL is a moiety that binds to mTOR. In certain embodiments, the EPL is a moiety that binds to PLK1. In certain embodiments, the EPL is a moiety that binds to CDK1. In certain embodiments, the EPL is a moiety that binds to CDK2. In certain embodiments, the EPL is a moiety that binds to CDK9. In certain embodiments, the EPL is a moiety that binds to BRD4. In certain embodiments, the EPL is a moiety that binds to AURKA. In certain embodiments, the EPL is a moiety that binds to AURKB. In certain embodiments, the EPL is a moiety that binds to MEK. In certain embodiments, the EPL is a moiety that binds to Src. In certain embodiments, the EPL is a moiety that binds to c-KIT. In certain embodiments, the EPL is a moiety that binds to KIF11. In certain embodiments, the EPL is a moiety that binds to HSP90. In certain embodiments, the EPL is a moiety that binds to tubulin. In certain embodiments, the EPL is a moiety that binds to proteasome. In certain embodiments, the EPL is a moiety that binds to topoisomerase. In certain embodiments, the EPL is a moiety that binds to HDAC.

A. Moiety for mTOR

In certain embodiments, the EPL is a moiety that binds to Mammalian Target of Rapamycin (mTOR). Exemplary moieties that bind mTOR are reported in the literature, including:

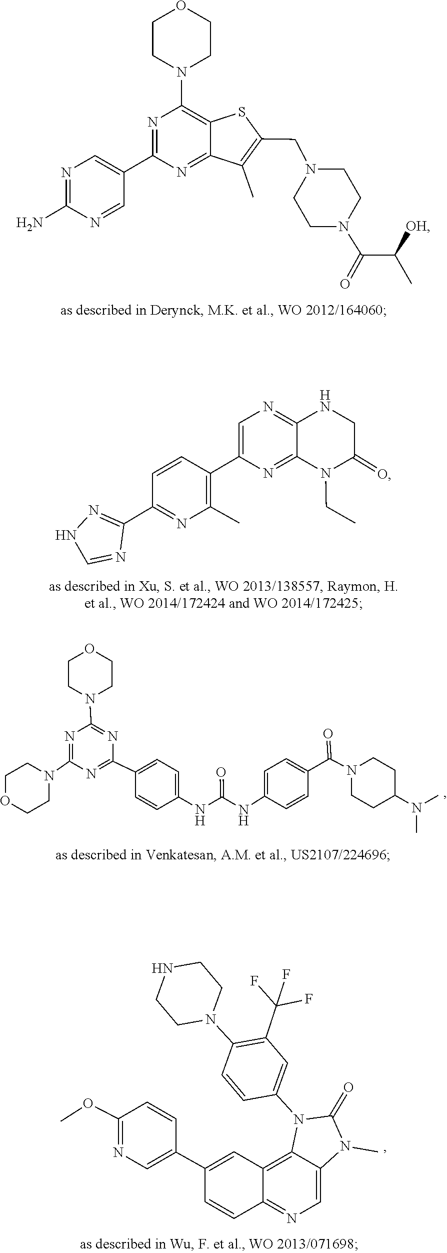

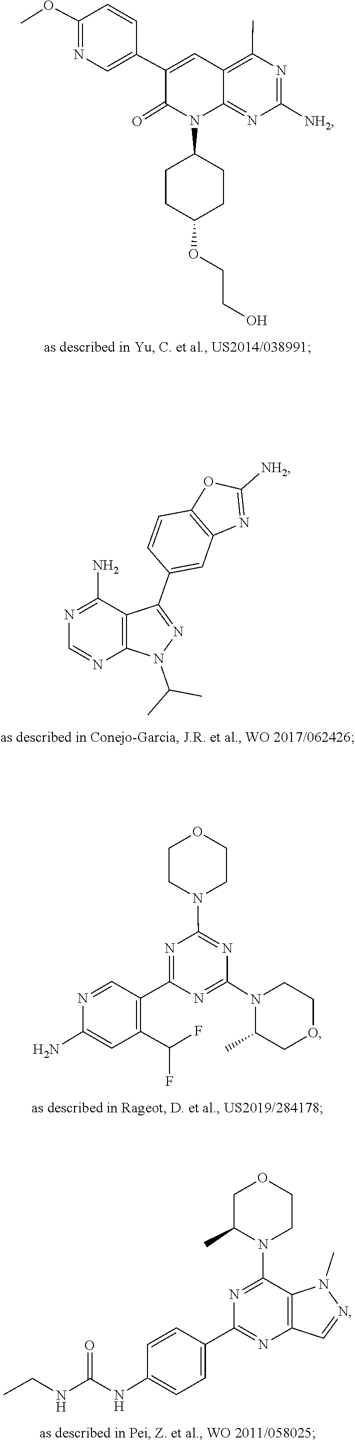 [SMILES: COc1ccc(-c2cc3c(C)nc(N)nc3n([C@H]3CC[C@H](OCCO)CC3)c2=O)cn1 ; CC(C)n1nc(-c2ccc3oc(N)nc3c2)c2c(N)ncnc21 ; C[C@H]1COCCN1c1nc(-c2cnc(N)cc2C(F)F)nc(N2CCOCC2)n1 ; CCNC(=O)Nc1ccc(-c2nc(N3CCOC[C@@H]3C)c3c(cnn3C)n2)cc1]

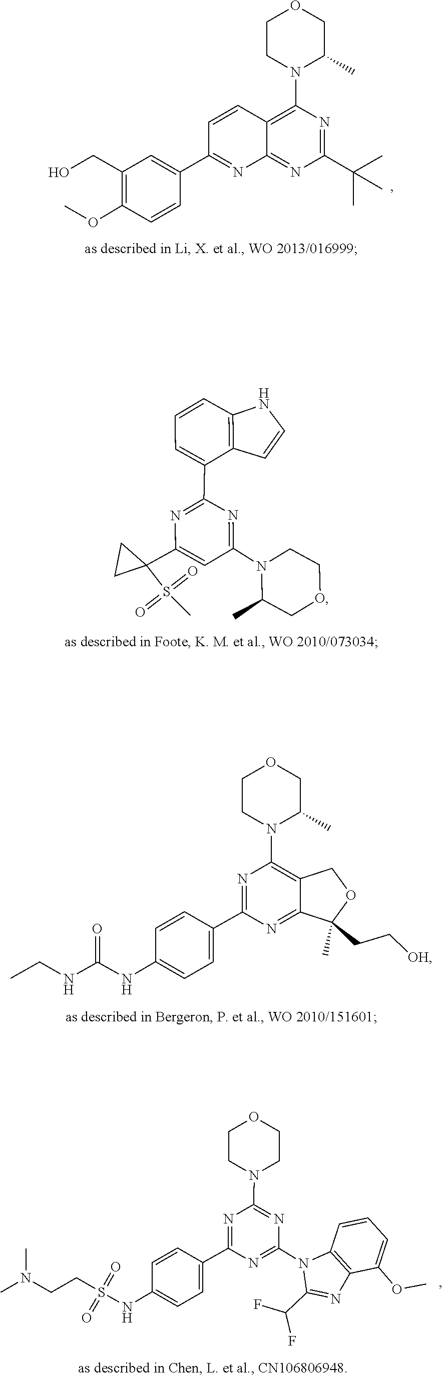

In certain embodiments, the EPL is a radical of one of the above compounds, which is attached to L through a modifiable oxygen, nitrogen, or carbon atom.

In certain embodiments, the EPL has the following formula:

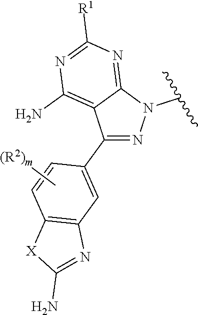

wherein:
R¹ and each R² represent independently for each occurrence halo, C₁-C₆ alkyl, C₁-C₆ haloalkyl, C₃-C₆ cycloalkyl, hydroxyl, C₁-C₆ alkoxy, or cyano;
R³ is hydrogen, C₁-C₆ alkyl, or C₃-C₆ cycloalkyl;
X is O, S, or N(R³); and
m is 0, 1, 2, or 3.

In certain embodiments, the EPL has the following formula:

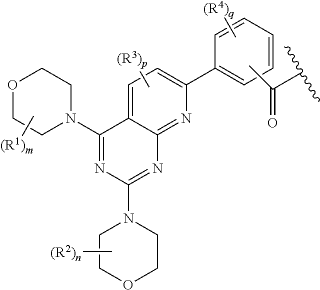

wherein:
R¹ and R² each represent independently for each occurrence halo, C₁-C₆ alkyl, C₁-C₆ haloalkyl, or C₃-C₆ cycloalkyl;
R³ and R⁴ each represent independently for each occurrence halo, C₁-C₆ alkyl, C₁-C₆ haloalkyl, C₃-C₆ cycloalkyl, hydroxyl, C₁-C₆ alkoxy, or cyano;
m, n, and q each represent independently 0, 1, 2, or 3; and
p is 0, 1, or 2.

In certain embodiments, the EPL is one of the following:

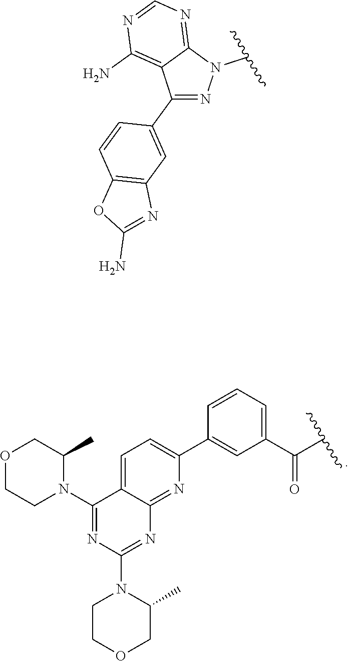 [SMILES: *n1nc(-c2ccc3oc(N)nc3c2)c2c(N)ncnc21 ; *C(=O)c1cccc(-c2ccc3c(N4CCOC[C@H]4C)nc(N4CCOC[C@H]4C)nc3n2)c1]

### B. Moiety for PLK1

In certain embodiments, the EPL is a moiety that binds to Polo Like Kinase I (PLK1). Exemplary compounds that bind to PLK1 are reported in the literature, including:

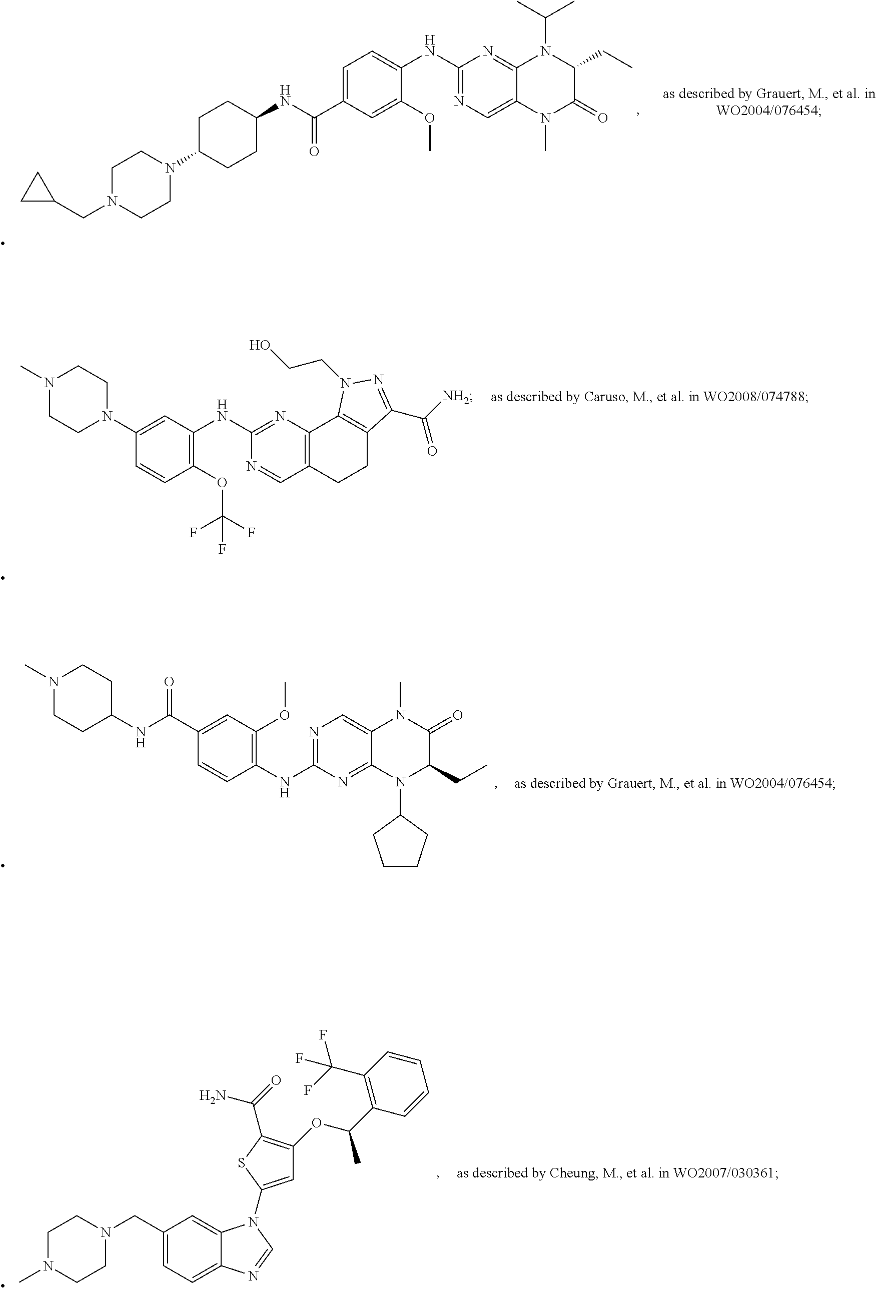

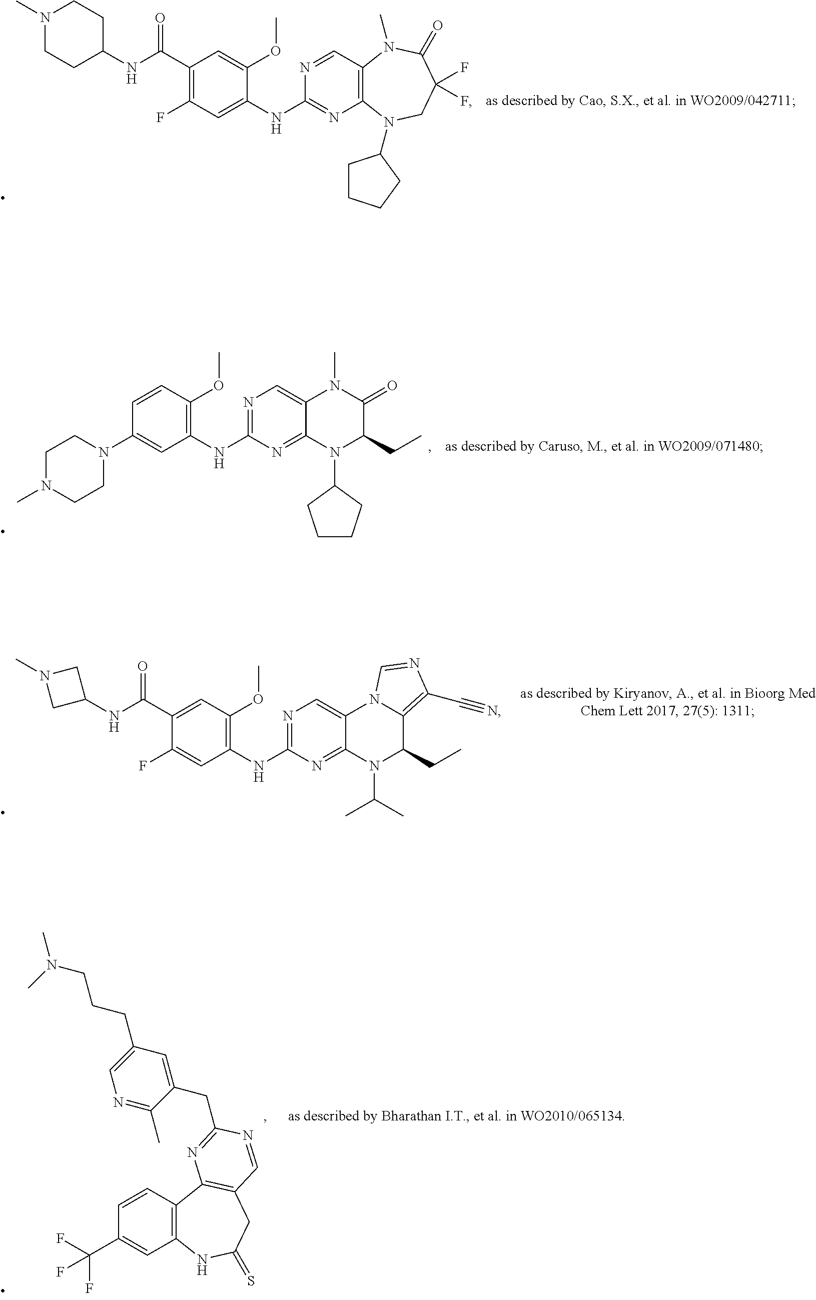

In certain embodiments, the EPL is a radical of one of the above compounds, which is attached to L through a modifiable oxygen, nitrogen, or carbon atom.

In certain embodiments, the EPL has the following formula:

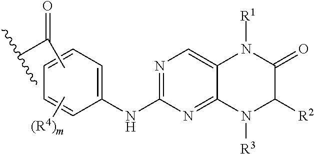

wherein:
R¹ and R² each represent independently C₁-C₆ alkyl, C₁-C₆ haloalkyl, or hydrogen;
R³ is C₃-C₆ cycloalkyl, C₁-C₆ alkyl, C₁-C₆ haloalkyl, or hydrogen;
R⁴ represents independently for each occurrence halo, C₁-C₆ alkyl, C₁-C₆ haloalkyl, C₃-C₆ cycloalkyl, hydroxyl, C₁-C₆ alkoxy, or cyano; and
m is 0, 1, 2, or 3.

In certain embodiments, the EPL is

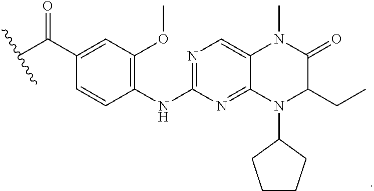 [SMILES: *C(=O)c1ccc(Nc2ncc3c(n2)N(C2CCCC2)C(CC)C(=O)N3C)c(OC)c1]

In certain embodiments, the EPL is

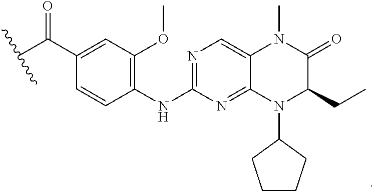 [SMILES: *C(=O)c1ccc(Nc2ncc3c(n2)N(C2CCCC2)[C@H](CC)C(=O)N3C)c(OC)c1]

In certain embodiments, the EPL has the following formula:

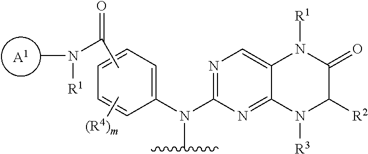

wherein:
R¹ and R² each represent independently for each occurrence C₁-C₆ alkyl, C₁-C₆ haloalkyl, or hydrogen;
R³ is C₃-C₆ cycloalkyl, C₁-C₆ alkyl, C₁-C₆ haloalkyl, or hydrogen;
R⁴ represents independently for each occurrence halo, C₁-C₆ alkyl, C₁-C₆ haloalkyl, C₃-C₆ cycloalkyl, hydroxyl, C₁-C₆ alkoxy, or cyano;
A¹ is a 3-7 membered saturated heterocyclyl containing 1, 2, or 3 heteroatoms independently selected from nitrogen, oxygen, and sulfur, wherein the heterocyclyl is optionally substituted with 1 or 2 occurrences of R⁴; and
m is 0, 1, 2, or 3.

In certain embodiments, the EPL has the following formula:

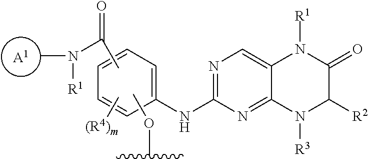

wherein:
R¹ and R² each represent independently for each occurrence C₁-C₆ alkyl, C₁-C₆ haloalkyl, or hydrogen;
R³ is C₃-C₆ cycloalkyl, C₁-C₆ alkyl, C₁-C₆ haloalkyl, or hydrogen;
R⁴ represents independently for each occurrence halo, C₁-C₆ alkyl, C₁-C₆ haloalkyl, C₃-C₆ cycloalkyl, hydroxyl, C₁-C₆ alkoxy, or cyano;
A¹ is a 3-7 membered saturated heterocyclyl containing 1, 2, or 3 heteroatoms independently selected from nitrogen, oxygen, and sulfur, wherein the heterocyclyl is optionally substituted with 1 or 2 occurrences of R⁴; and
m is 0, 1, 2, or 3.

In certain embodiments, A¹ is piperidinyl substituted with 1 or 2 occurrences of R⁴.

In certain embodiments, the EPL is one of the following:

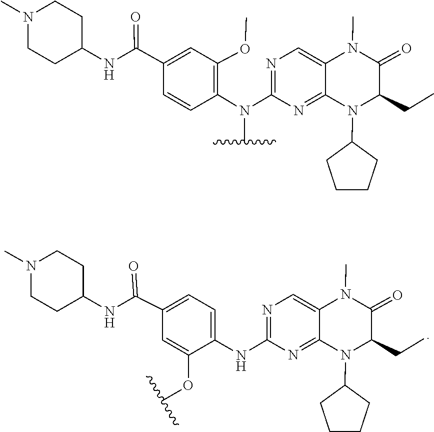

### C. Moiety for CDK1

In certain embodiments, the EPL is a moiety that binds to Cyclin-Dependent Kinase 1 (CDK1). Exemplary compounds that bind to CDK1 are reported in the literature, including:

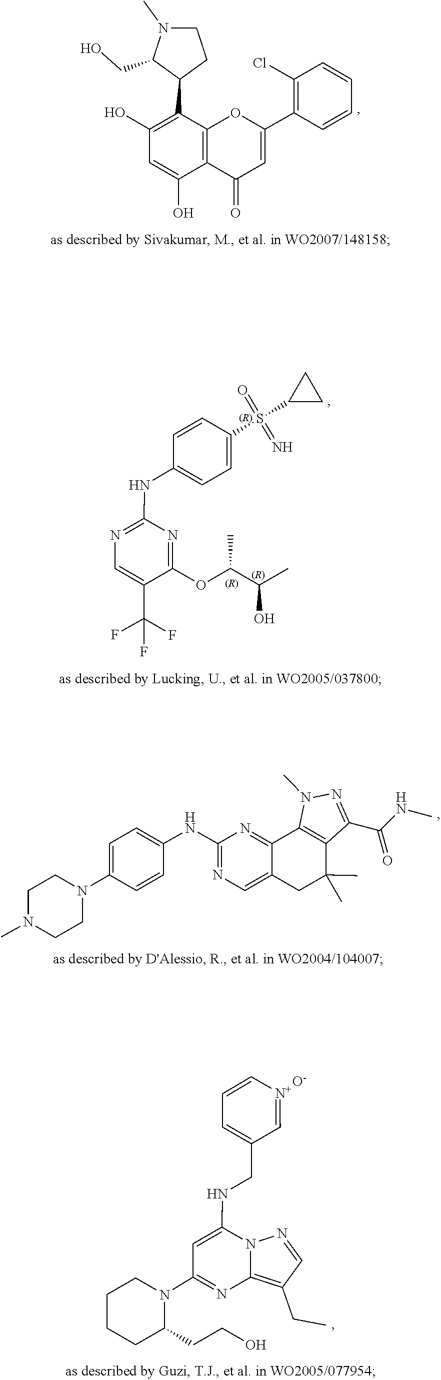

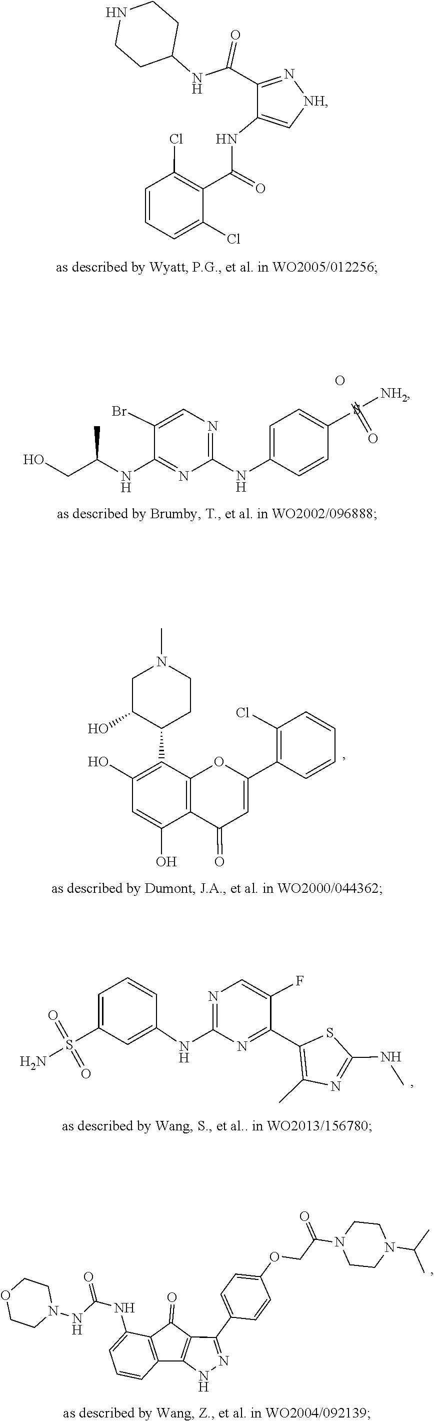

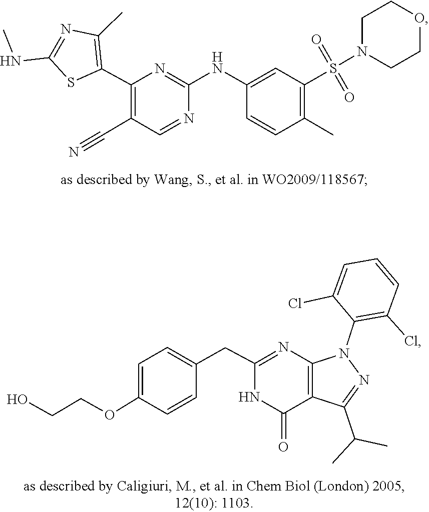 [SMILES: CNc1nc(C)c(-c2nc(Nc3ccc(C)c(S(=O)(=O)N4CCOCC4)c3)ncc2C#N)s1 ; CC(C)c1nn(-c2c(Cl)cccc2Cl)c2nc(Cc3ccc(OCCO)cc3)[nH]c(=O)c12]

In certain embodiments, the EPL is a radical of one of the above compounds, which is attached to L through a modifiable oxygen, nitrogen, or carbon atom.

In certain embodiments, the EPL has the following formula:

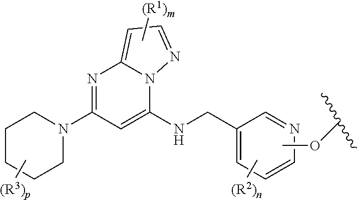

wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

In certain embodiments, the EPL has the following formula:

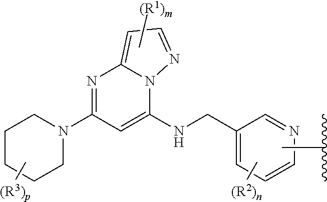

wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

In certain embodiments, the EPL has the following formula:

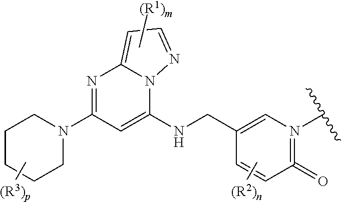

wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

In certain embodiments, the EPL has the following formula:

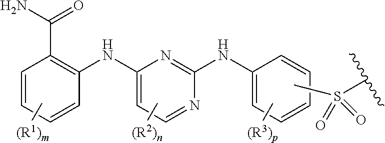

wherein:
R¹, R² and R³ each represent independently for each occurrence halo, C₁-C₆ alkyl, C₁-C₆ haloalkyl, C₃-C₆ cycloalkyl, hydroxyl, C₁-C₆ alkoxy, or cyano;
m and p each represent independently 0, 1, or 2; and
n is 0, 1 or 2.

In certain embodiments, the EPL is one of the following:

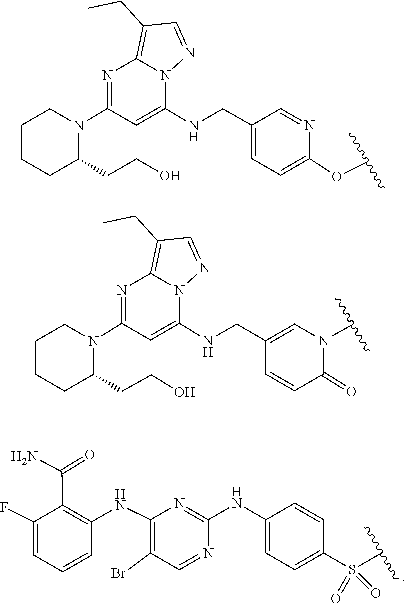 [SMILES: *Oc1ccc(CNc2cc(N3CCCC[C@H]3CCO)nc3c(CC)cnn23)cn1 ; *n1cc(CNc2cc(N3CCCC[C@H]3CCO)nc3c(CC)cnn23)ccc1=O ; *S(=O)(=O)c1ccc(Nc2ncc(Br)c(Nc3cccc(F)c3C(N)=O)n2)cc1]

In certain embodiments, the EPL is one of the following:

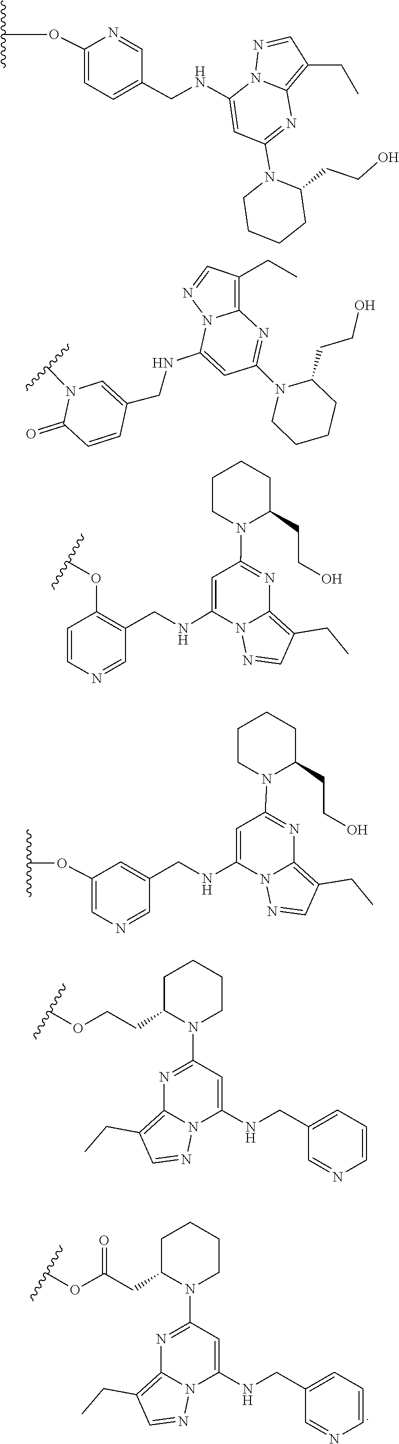

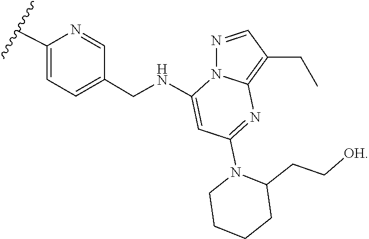 [SMILES: *c1ccc(CNc2cc(N3CCCCC3CCO)nc3c(CC)cnn23)cn1]

In certain embodiments, the EPL is

### D. Moiety for CDK2

In certain embodiments, the EPL is a moiety that binds to Cyclin-Dependent Kinase 2 (CDK2). Exemplary compounds that bind to CDK2 are reported in the literature, including:

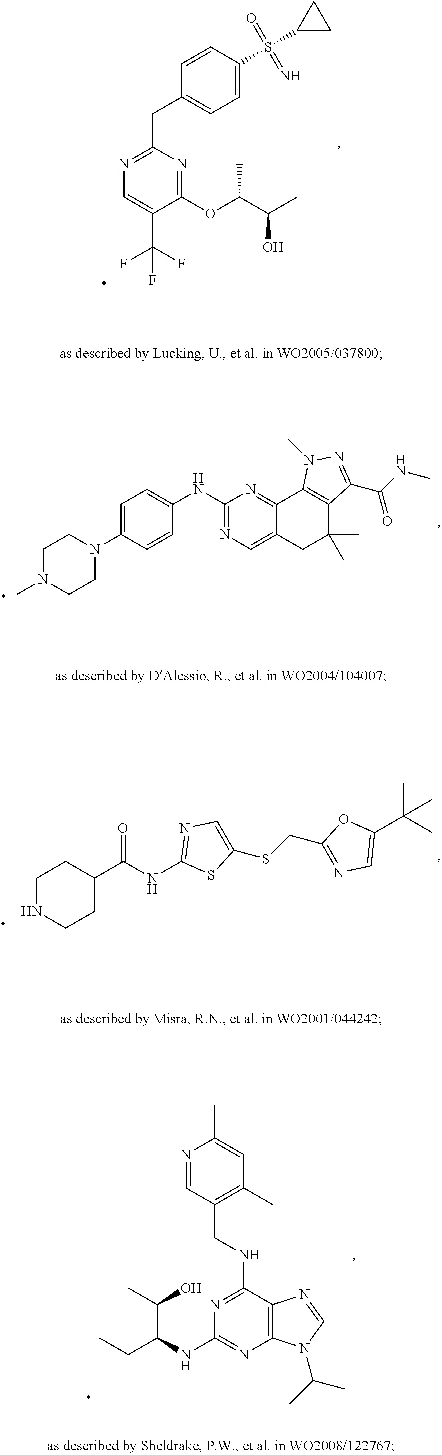

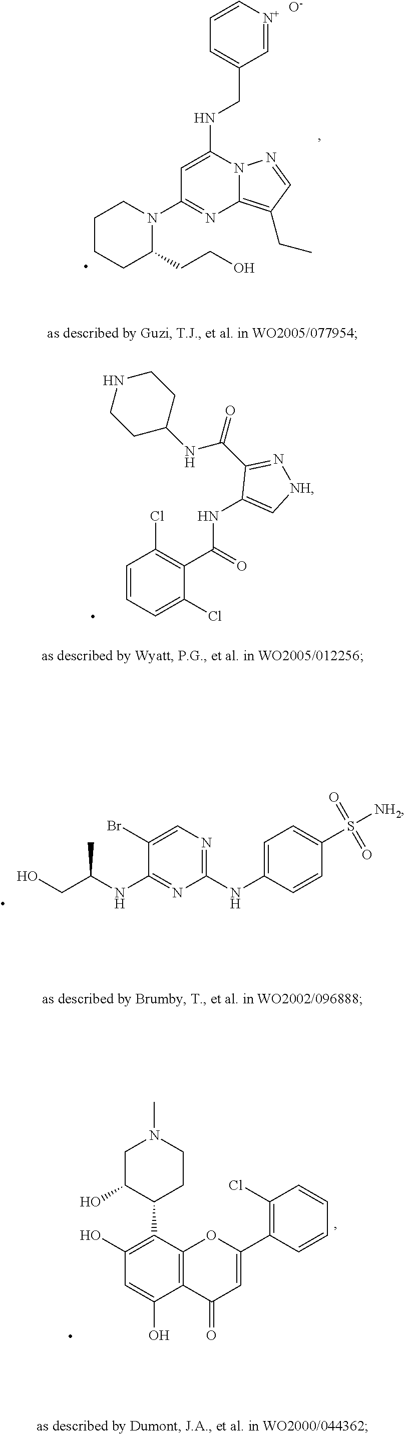

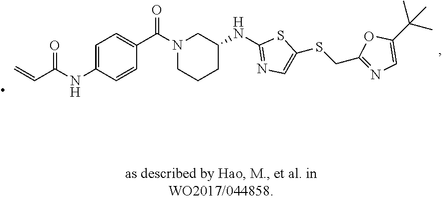 [SMILES: C=CC(=O)Nc1ccc(C(=O)N2CCC[C@@H](Nc3ncc(SCc4ncc(C(C)(C)C)o4)s3)C2)cc1]

In certain embodiments, the EPL is a radical of one of the above compounds, which is attached to L through a modifiable oxygen, nitrogen, or carbon atom.

In certain embodiments, the EPL has the following formula:

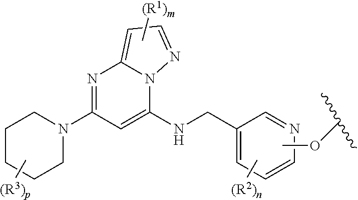

wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

In certain embodiments, the EPL has the following formula:

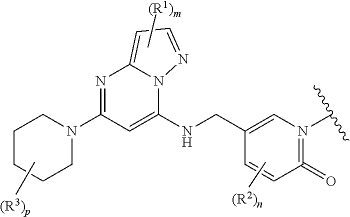

wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

In certain embodiments, the EPL is one of the following:

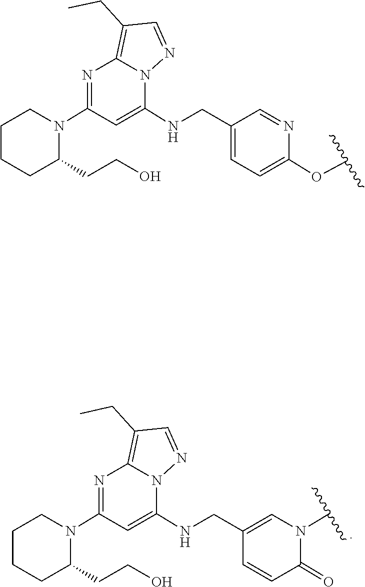 [SMILES: *Oc1ccc(CNc2cc(N3CCCC[C@H]3CCO)nc3c(CC)cnn23)cn1 ; *n1cc(CNc2cc(N3CCCC[C@H]3CCO)nc3c(CC)cnn23)ccc1=O]

### E. Moiety for CDK9

In certain embodiments, the EPL is a moiety that binds to Cyclin-Dependent Kinase 9 (CDK9). Exemplary compounds that bind to CDK9 are reported in the literature, including:

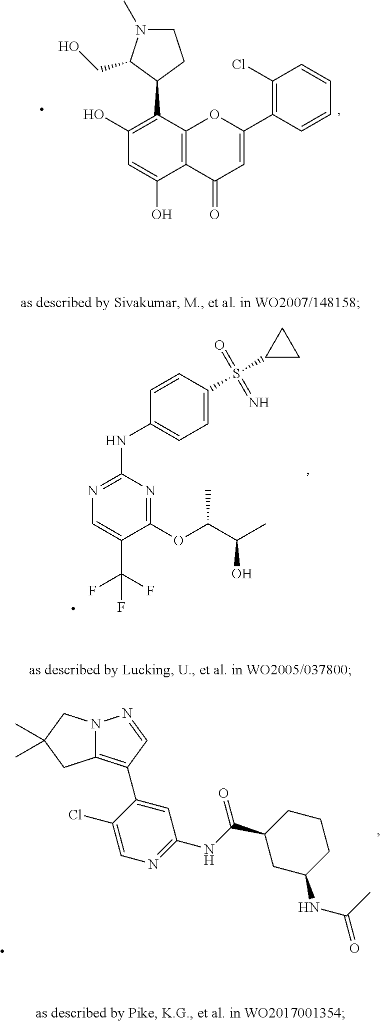 [SMILES: CN1CC[C@@H](c2c(O)cc(O)c3c(=O)cc(-c4ccccc4Cl)oc23)[C@@H]1CO ; C[C@@H](O)[C@@H](C)Oc1nc(Nc2ccc([S@](=N)(=O)C3CC3)cc2)ncc1C(F)(F)F ; CC(=O)N[C@@H]1CCC[C@H](C(=O)Nc2cc(-c3cnn4c3CC(C)(C)C4)c(Cl)cn2)C1]

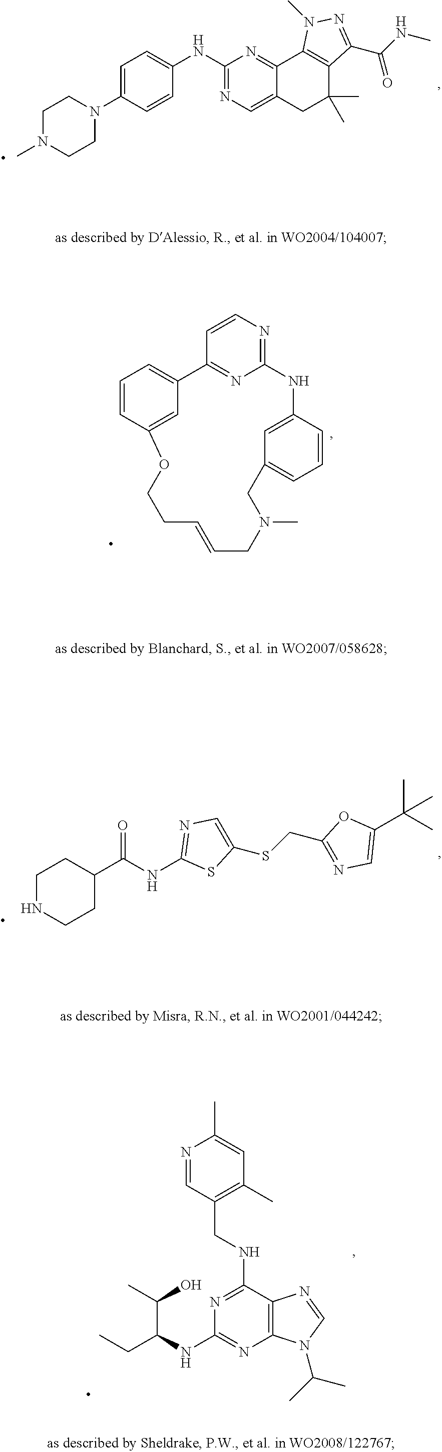

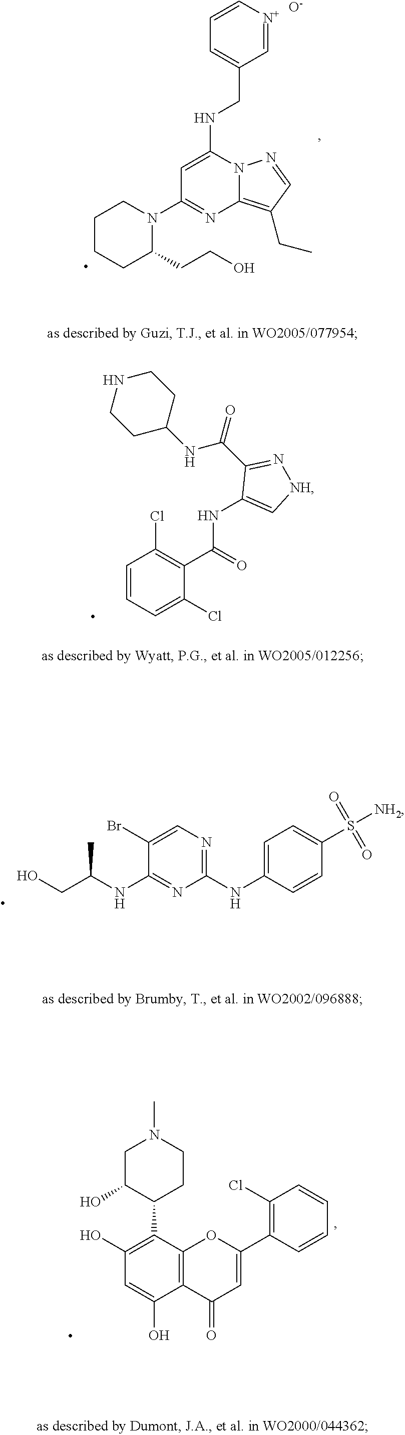

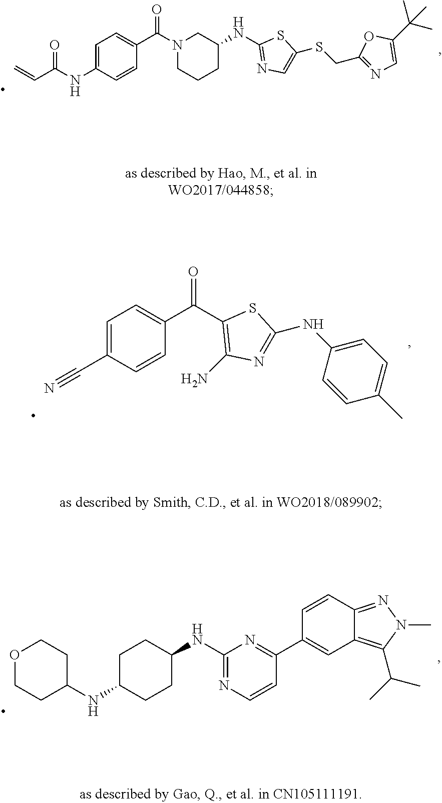 [SMILES: C=CC(=O)Nc1ccc(C(=O)N2CCC[C@@H](Nc3ncc(SCc4ncc(C(C)(C)C)o4)s3)C2)cc1 ; Cc1ccc(Nc2nc(N)c(C(=O)c3ccc(C#N)cc3)s2)cc1 ; CC(C)c1c2cc(-c3ccnc(N[C@H]4CC[C@H](NC5CCOCC5)CC4)n3)ccc2nn1C]

In certain embodiments, the EPL is a radical of one of the above compounds, which is attached to L through a modifiable oxygen, nitrogen, or carbon atom.

In certain embodiments, the EPL has the following formula:

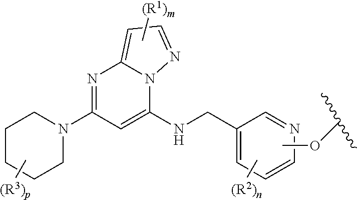

wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

In certain embodiments, the EPL has the following formula:

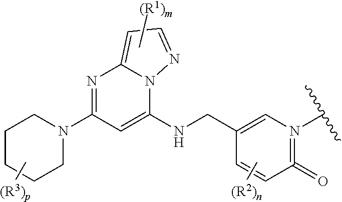

wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

In certain embodiments, the EPL is one of the following:

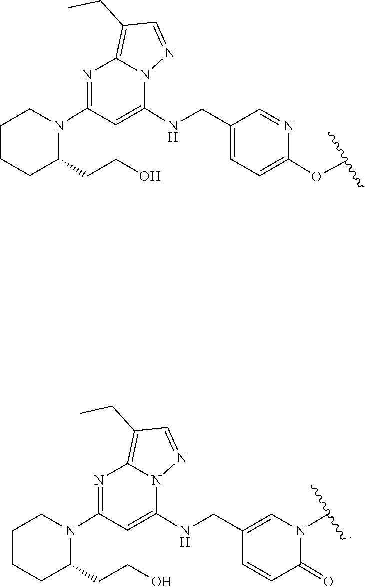 [SMILES: *Oc1ccc(CNc2cc(N3CCCC[C@H]3CCO)nc3c(CC)cnn23)cn1 ; *n1cc(CNc2cc(N3CCCC[C@H]3CCO)nc3c(CC)cnn23)ccc1=O]

### F. Moiety for BRD4

In certain embodiments, the EPL is a moiety that binds to bromodomain-containing protein 4 (BRD4). Exemplary compounds that bind to BRD4 are reported in the literature, including:

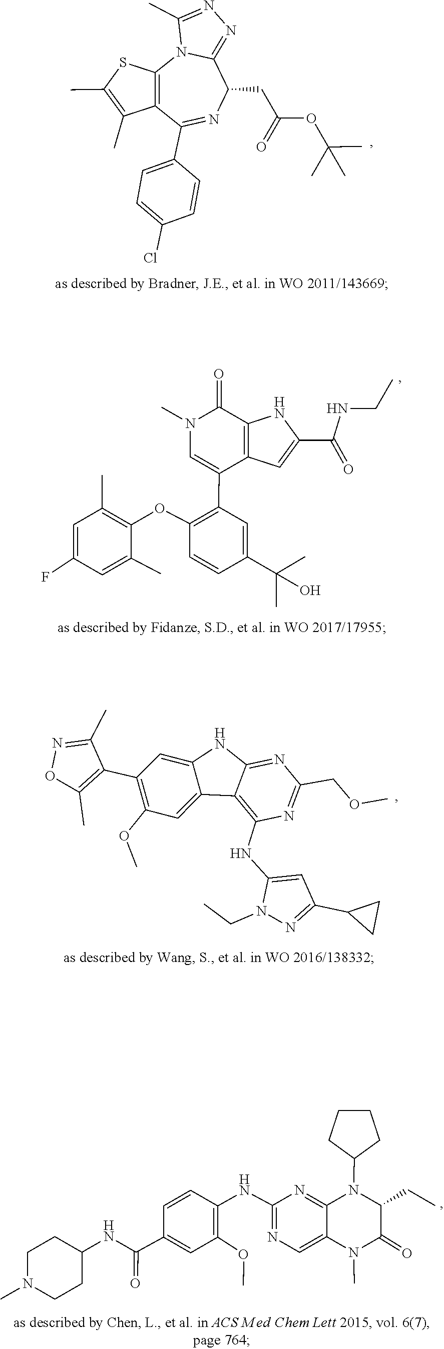

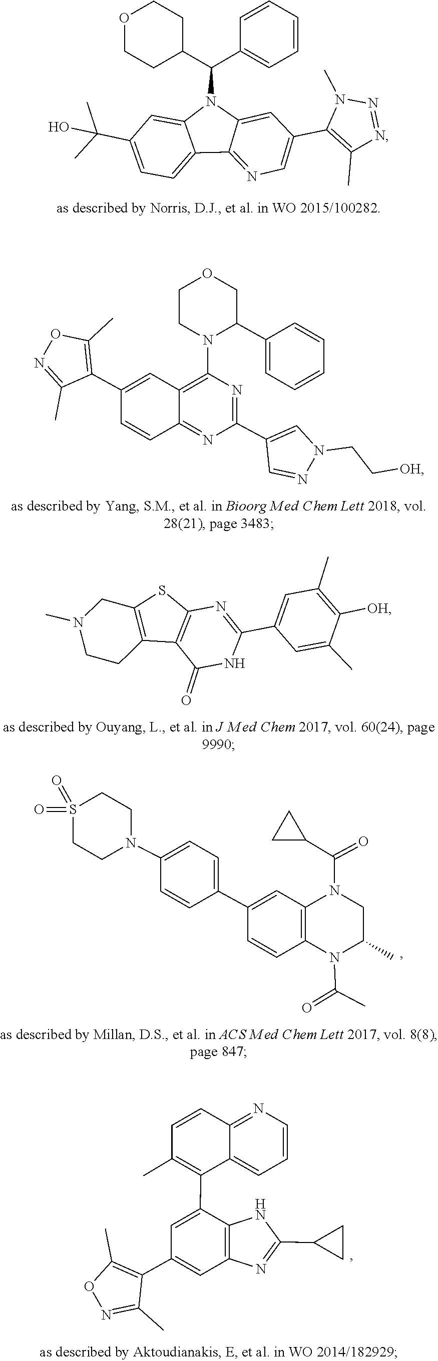 [SMILES: Cc1nnn(C)c1-c1cnc2c3ccc(C(C)(C)O)cc3n([C@H](c3ccccc3)C3CCOCC3)c2c1 ; Cc1noc(C)c1-c1ccc2nc(-c3cnn(CCO)c3)nc(N3CCOCC3c3ccccc3)c2c1 ; Cc1cc(-c2nc3sc4c(c3c(=O)[nH]2)CCN(C)C4)cc(C)c1O ; CC(=O)N1c2ccc(-c3ccc(N4CCS(=O)(=O)CC4)cc3)cc2N(C(=O)C2CC2)C[C@@H]1C ; Cc1ccc2ncccc2c1-c1cc(-c2c(C)noc2C)cc2nc(C3CC3)[nH]c12]

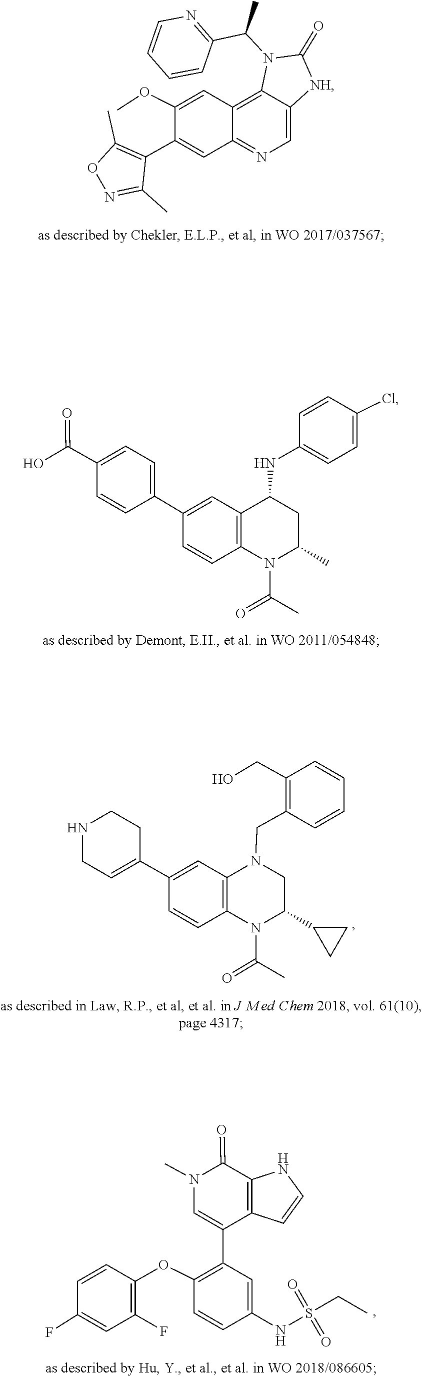 [SMILES: COc1cc2c(cc1-c1c(C)noc1C)ncc1[nH]c(=O)n([C@H](C)c3ccccn3)c12 ; CC(=O)N1c2ccc(-c3ccc(C(=O)O)cc3)cc2[C@H](Nc2ccc(Cl)cc2)C[C@@H]1C ; CC(=O)N1c2ccc(C3=CCNCC3)cc2N(Cc2ccccc2CO)C[C@@H]1C1CC1 ; CCS(=O)(=O)Nc1ccc(Oc2ccc(F)cc2F)c(-c2cn(C)c(=O)c3[nH]ccc23)c1]

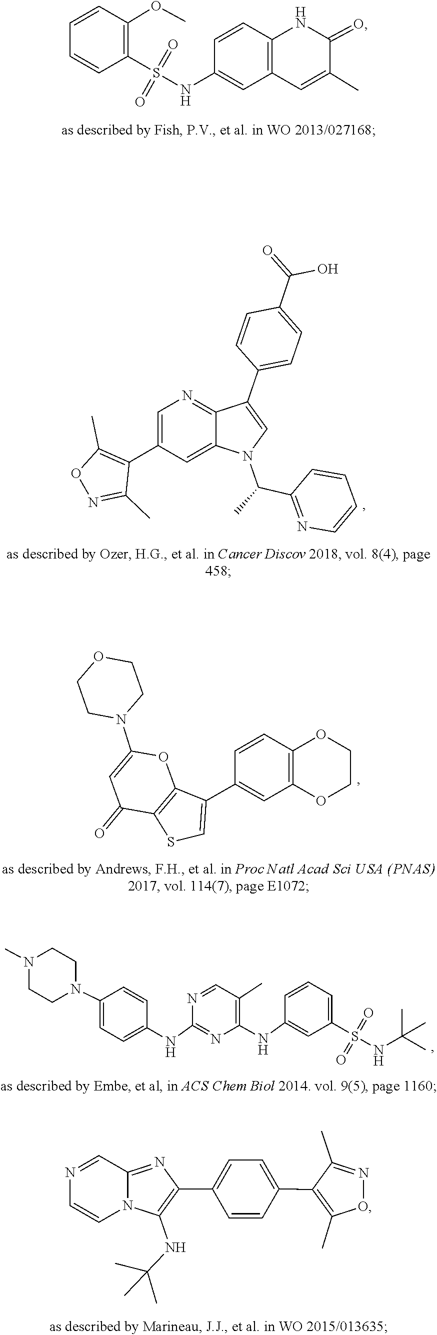 [SMILES: COc1ccccc1S(=O)(=O)Nc1ccc2[nH]c(=O)c(C)cc2c1 ; Cc1noc(C)c1-c1cnc2c(-c3ccc(C(=O)O)cc3)cn([C@@H](C)c3ccccn3)c2c1 ; O=c1cc(N2CCOCC2)oc2c(-c3ccc4c(c3)OCCO4)csc12 ; Cc1cnc(Nc2ccc(N3CCN(C)CC3)cc2)nc1Nc1cccc(S(=O)(=O)NC(C)(C)C)c1 ; Cc1noc(C)c1-c1ccc(-c2nc3cnccn3c2NC(C)(C)C)cc1]

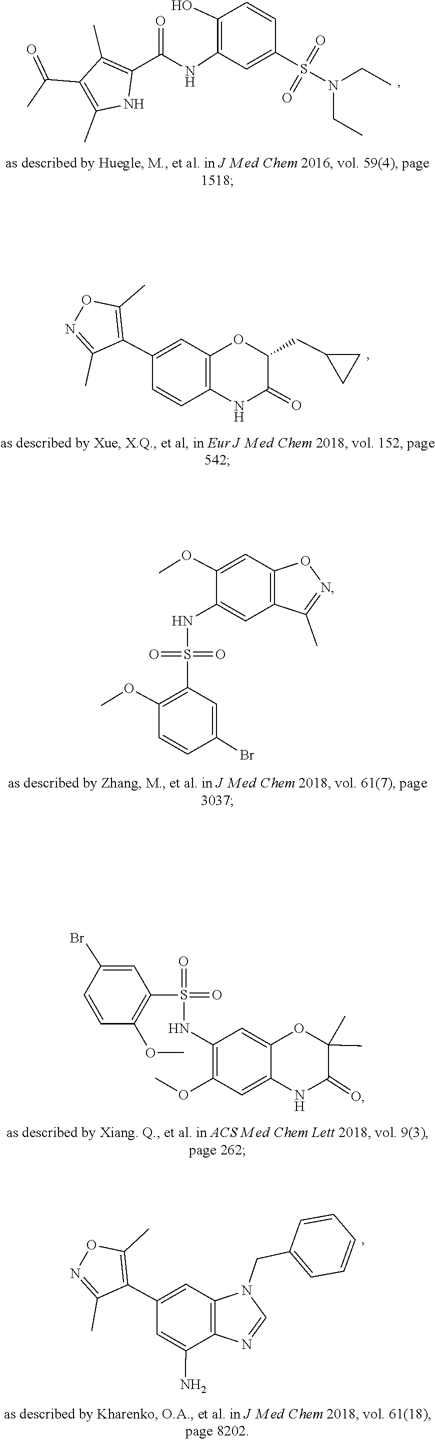

In certain embodiments, the EPL is a radical of one of the above compounds, which is attached to L through a modifiable oxygen, nitrogen, or carbon atom.

In certain embodiments, the EPL has the following formula:

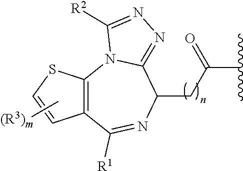

wherein:
R¹ is phenyl, C₃-C₈ cycloalkyl, or 5-6 membered heteroaryl containing 1, 2, or 3 heteroatoms independently selected from nitrogen, oxygen, and sulfur; each of which is optionally substituted with 1, 2, or 3 substituents independently selected from halo, C₁-C₆ alkyl, C₁-C₆ haloalkyl, C₃-C₆ cycloalkyl, hydroxyl, C₁-C₆ alkoxy, or cyano;
R² and each R³ represent independently for each occurrence halo, C₁-C₆ alkyl, C₁-C₆ haloalkyl, C₃-C₆ cycloalkyl, hydroxyl, C₁-C₆ alkoxy, or cyano;
m is 0, 1, or 2; and
n is 0, 1, 2, 3, or 4.

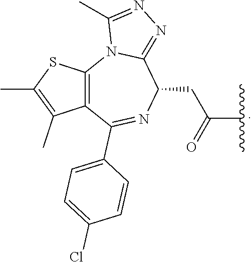 [SMILES: *C(=O)C[C@@H]1N=C(c2ccc(Cl)cc2)c2c(sc(C)c2C)-n2c(C)nnc21]

In certain embodiments, the EPL is

In certain embodiments, the EPL has the following formula:

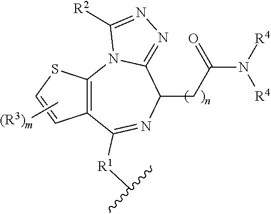

wherein:
R¹ is phenylene substituted with 0, 2, or 3 substituents independently selected from halo, C₁-C₆ alkyl, C₁-C₆ haloalkyl, C₃-C₆ cycloalkyl, hydroxyl, C₁-C₆ alkoxy, or cyano;
R² and each R³ represent independently for each occurrence halo, C₁-C₆ alkyl, C₁-C₆ haloalkyl, C₃-C₆ cycloalkyl, hydroxyl, C₁-C₆ alkoxy, or cyano;
R⁴ represents independently for each occurrence hydrogen, C₁-C₆ alkyl, or C₃-C₆ cycloalkyl;
m is 0, 1, or 2; and
n is 0, 1, 2, 3, or 4.

In certain embodiments, the EPL is

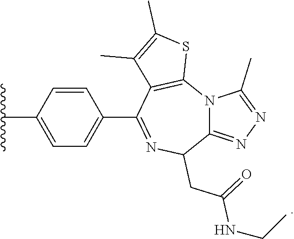 [SMILES: *c1ccc(C2=NC(CC(=O)NCC)c3nnc(C)n3-c3sc(C)c(C)c32)cc1]

In certain embodiments, R¹ is phenylene. In certain embodiments, R² and R³ are C₁-C₆ alkyl. In certain embodiments, R² is C₁-C₆ alkyl. In certain embodiments, R³ is C₁-C₆ alkyl. In certain embodiments, R⁴ is hydrogen or C₁-C₆ alkyl. In certain embodiments, R⁴ is hydrogen. In certain embodiments, R⁴ is C₁-C₆ alkyl.

In certain embodiments, n is 0. In certain embodiments, n is 1. In certain embodiments, n is 2. In certain embodiments, n is 3.

In certain embodiments, the EPL is

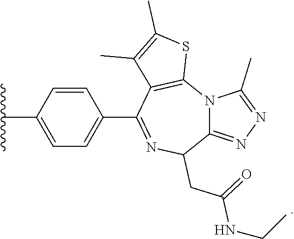 [SMILES: *c1ccc(C2=NC(CC(=O)NCC)c3nnc(C)n3-c3sc(C)c(C)c32)cc1]

In certain embodiments, the EPL has the following formula:

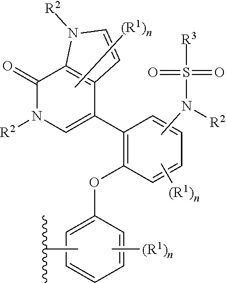

wherein:
R¹ represents independently for each occurrence halo, C₁-C₆ alkyl, C₁-C₆ haloalkyl, C₃-C₆ cycloalkyl, or hydroxyl;
R² represents independently for each occurrence hydrogen, C₁-C₆ alkyl, or C₃-C₆ cycloalkyl;
R³ is C₁-C₆ alkyl or C₃-C₆ cycloalkyl; and
n represents independently for each occurrence 0, 1, 2, or 3.

In certain embodiments, R¹ represents independently for each occurrence halo or C₁-C₆ alkyl. In certain embodiments, R² is hydrogen. In certain embodiments, n is 0. In certain embodiments, n is 1. In certain embodiments, n is 2. In certain embodiments, n is 3.

In certain embodiments, the EPL is

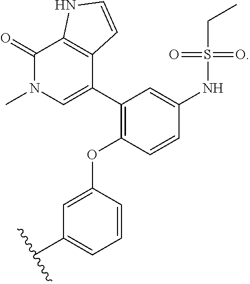 [SMILES: *c1cccc(Oc2ccc(NS(=O)(=O)CC)cc2-c2cn(C)c(=O)c3[nH]ccc23)c1]

### G. Moiety for AURKA

In certain embodiments, the EPL is a moiety that binds to Aurora Kinase A (AURKA). Exemplary compounds that bind to AURKA are reported in the literature, including:

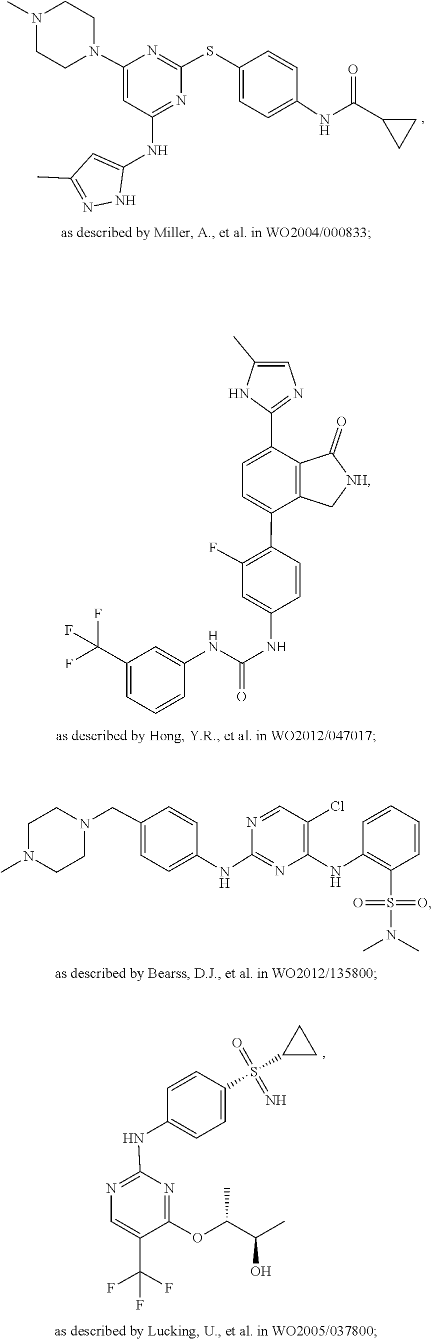 [SMILES: Cc1cc(Nc2cc(N3CCN(C)CC3)nc(Sc3ccc(NC(=O)C4CC4)cc3)n2)[nH]n1 ; Cc1cnc(-c2ccc(-c3ccc(NC(=O)Nc4cccc(C(F)(F)F)c4)cc3F)c3c2C(=O)NC3)[nH]1 ; CN1CCN(Cc2ccc(Nc3ncc(Cl)c(Nc4ccccc4S(=O)(=O)N(C)C)n3)cc2)CC1 ; C[C@@H](O)[C@@H](C)Oc1nc(Nc2ccc([S@](=N)(=O)C3CC3)cc2)ncc1C(F)(F)F]

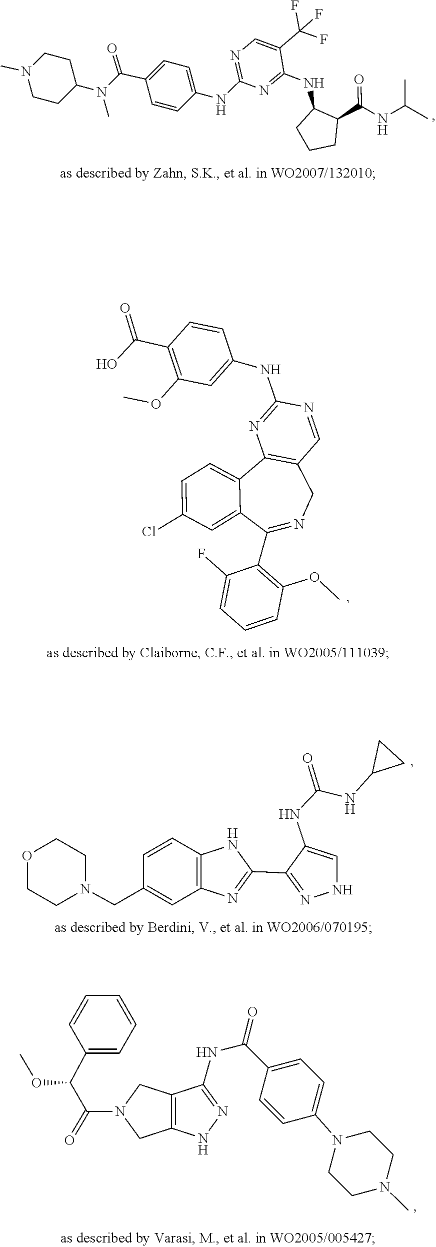

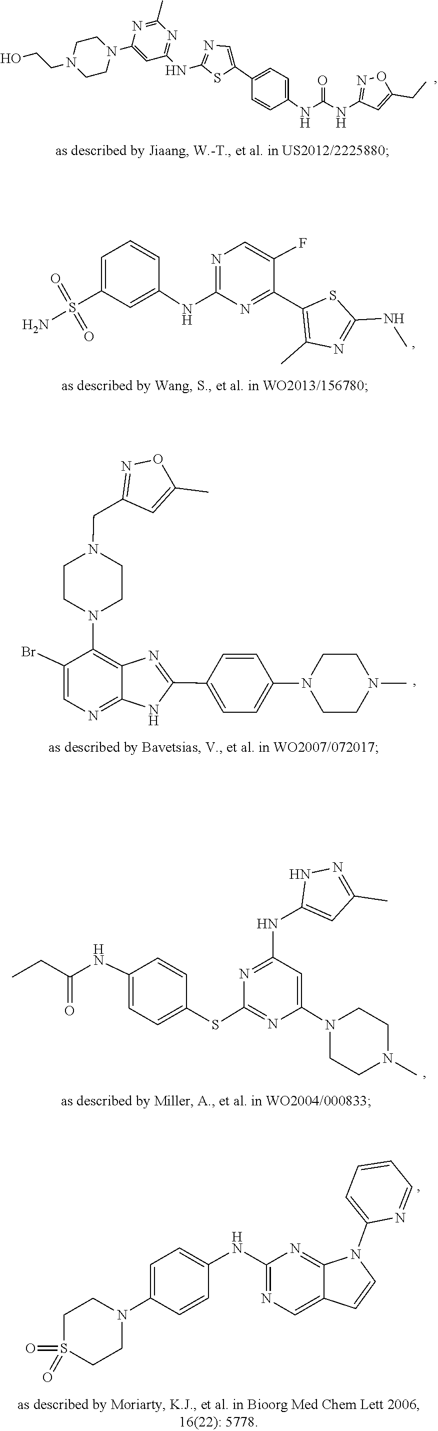

In certain embodiments, the EPL is a radical of one of the above compounds, which is attached to L through a modifiable oxygen, nitrogen, or carbon atom.

In certain embodiments, the EPL has the following formula:

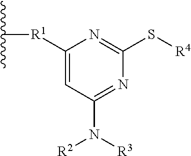

wherein:
R¹ is 4-7 membered, saturated heterocyclylene containing 1, 2, or 3 heteroatoms independently selected from nitrogen, oxygen, and sulfur;
R² is a 5-6membered heteroaryl containing 1, 2, or 3 heteroatoms independently selected from nitrogen, oxygen, and sulfur; wherein the heteroaryl is optionally substituted with 1 or 2 substituents independently selected from halo, C₁-C₆ alkyl, C₁-C₆ haloalkyl, C₃-C₆ cycloalkyl, hydroxyl, C₁-C₆ alkoxy, or cyano;
R³ represents independently for each occurrence H or C₁-C₆ alkyl; and
R⁴ is phenyl or a 5-6 membered heteroaryl containing 1, 2, or 3 heteroatoms independently selected from nitrogen, oxygen, and sulfur; each of which is optionally substituted with 1, 2, or 3 substituents independently selected from —N(R³)C(O)—(C₃-C₆ cycloalkyl), —N(R³)C(O)—(C₁-C₆ alkyl), halo, C₁-C₆ alkyl, C₁-C₆ haloalkyl, C₃-C₆ cycloalkyl, hydroxyl, C₁-C₆ alkoxy, or cyano.

In certain embodiments, the EPL has the following formula:

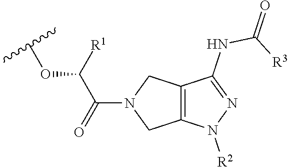

wherein:
R¹ is phenyl or a 5-6 membered heteroaryl containing 1, 2, or 3 heteroatoms independently selected from nitrogen, oxygen, and sulfur; each of which is optionally substituted with 1, 2, or 3 substituents independently selected from halo, C₁-C₆ alkyl, C₁-C₆ haloalkyl, C₃-C₆ cycloalkyl, hydroxyl, C₁-C₆ alkoxy, or cyano;
R² is -(phenylene)-(4-7 membered, saturated heterocyclyl containing 1, 2, or 3 heteroatoms independently selected from nitrogen, oxygen, and sulfur); wherein the heterocyclyl is optionally substituted with 1 or 2 substituents independently selected from halo, C₁-C₆ alkyl, C₁-C₆ haloalkyl, C₃-C₆ cycloalkyl, hydroxyl, C₁-C₆ alkoxy, or cyano; and
R³ represents independently for each occurrence H or C₁-C₆ alkyl.

In certain embodiments, the EPL is one of the following:

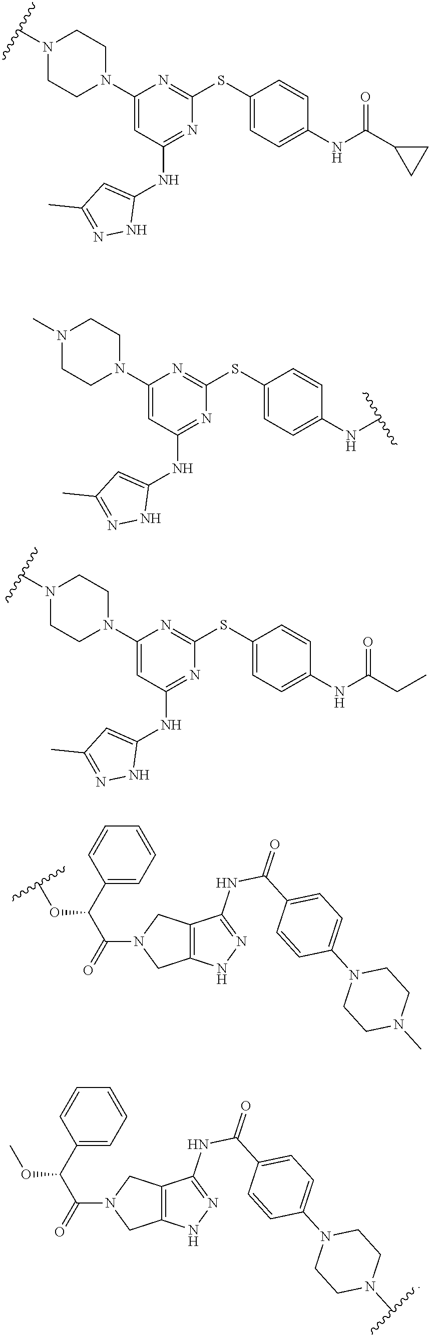

### H. Moiety for AURKB

In certain embodiments, the EPL is a moiety that binds to Aurora Kinase B (AURKB). Exemplary compounds that bind to AURKB are reported in the literature, including:

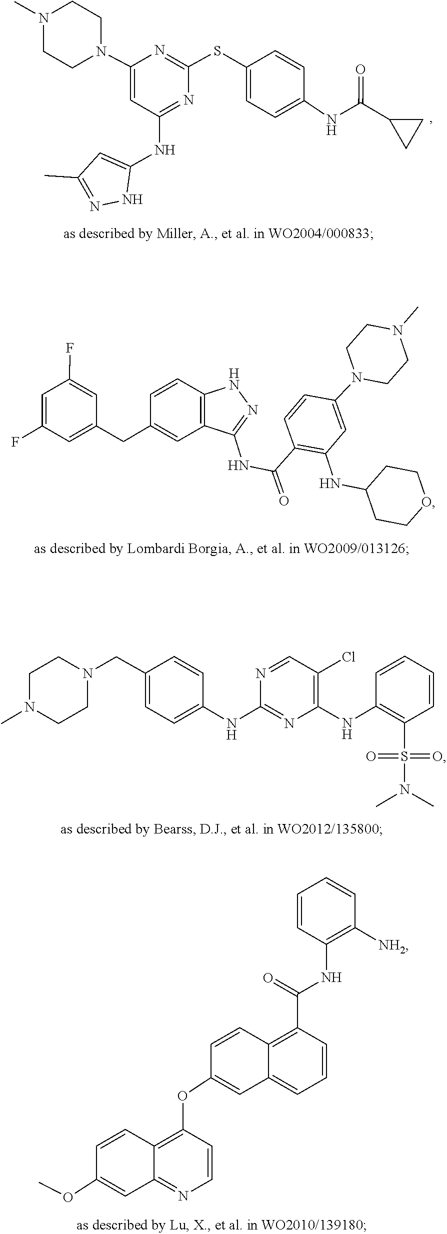 [SMILES: Cc1cc(Nc2cc(N3CCN(C)CC3)nc(Sc3ccc(NC(=O)C4CC4)cc3)n2)[nH]n1 ; CN1CCN(c2ccc(C(=O)Nc3n[nH]c4ccc(Cc5cc(F)cc(F)c5)cc34)c(NC3CCOCC3)c2)CC1 ; CN1CCN(Cc2ccc(Nc3ncc(Cl)c(Nc4ccccc4S(=O)(=O)N(C)C)n3)cc2)CC1 ; COc1ccc2c(Oc3ccc4c(C(=O)Nc5ccccc5N)cccc4c3)ccnc2c1]

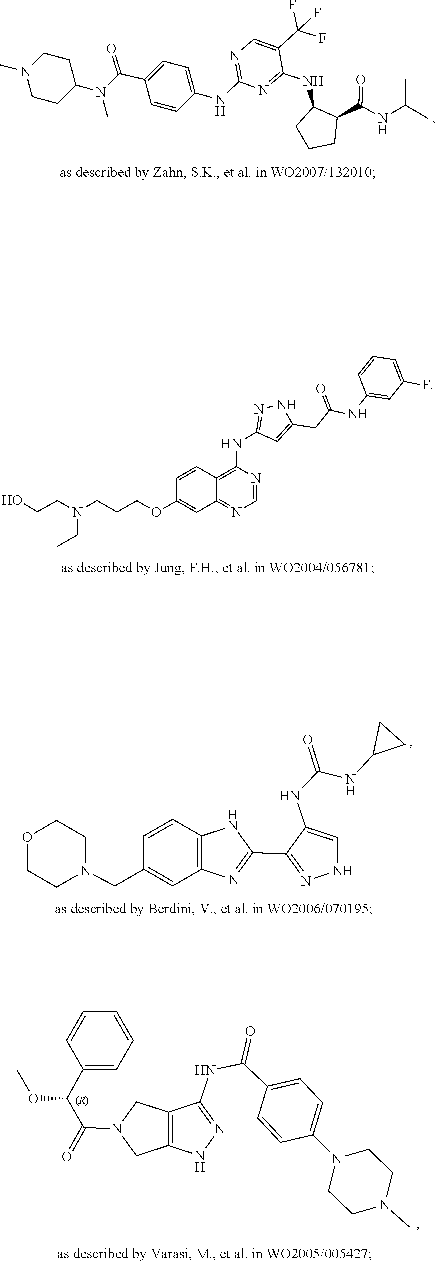

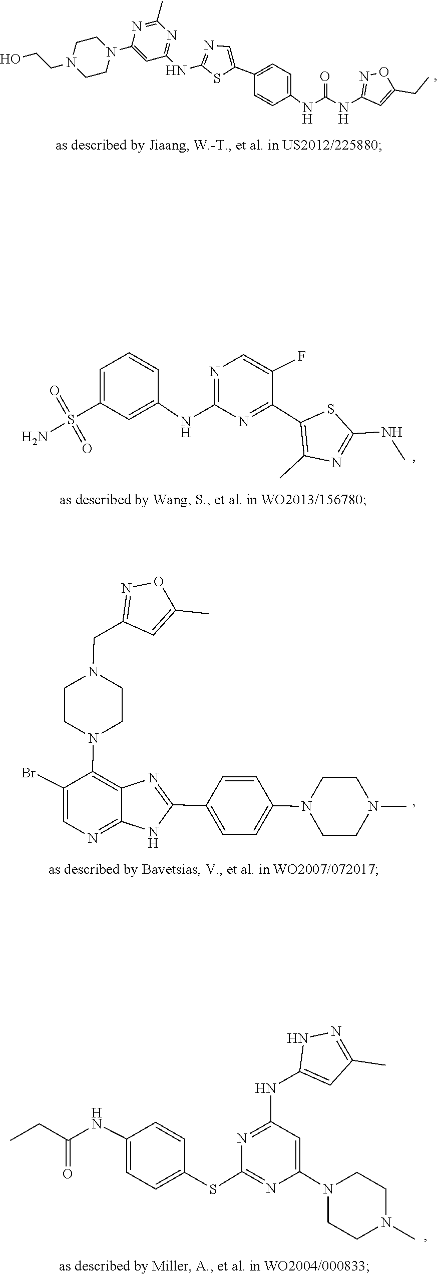

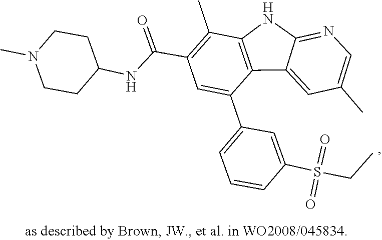 [SMILES: CCS(=O)(=O)c1cccc(-c2cc(C(=O)NC3CCN(C)CC3)c(C)c3[nH]c4ncc(C)cc4c23)c1]

In certain embodiments, the EPL is a radical of one of the above compounds, which is attached to L through a modifiable oxygen, nitrogen, or carbon atom.

In certain embodiments, the EPL has the following formula:

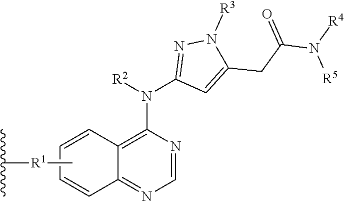

wherein:
R¹ is 4-10 membered heteroalkylene;
R², R³, and R⁵ are independently H or C₁-C₆ alkyl; and
R⁴ is phenyl or a 5-6 membered heteroaryl containing 1, 2, or 3 heteroatoms independently selected from nitrogen, oxygen, and sulfur; each of which is optionally substituted with 1, 2, or 3 substituents independently selected from halo, C₁-C₆ alkyl, C₁-C₆ haloalkyl, C₃-C₆ cycloalkyl, hydroxyl, C₁-C₆ alkoxy, or cyano.

In certain embodiments, the EPL is one of the following:

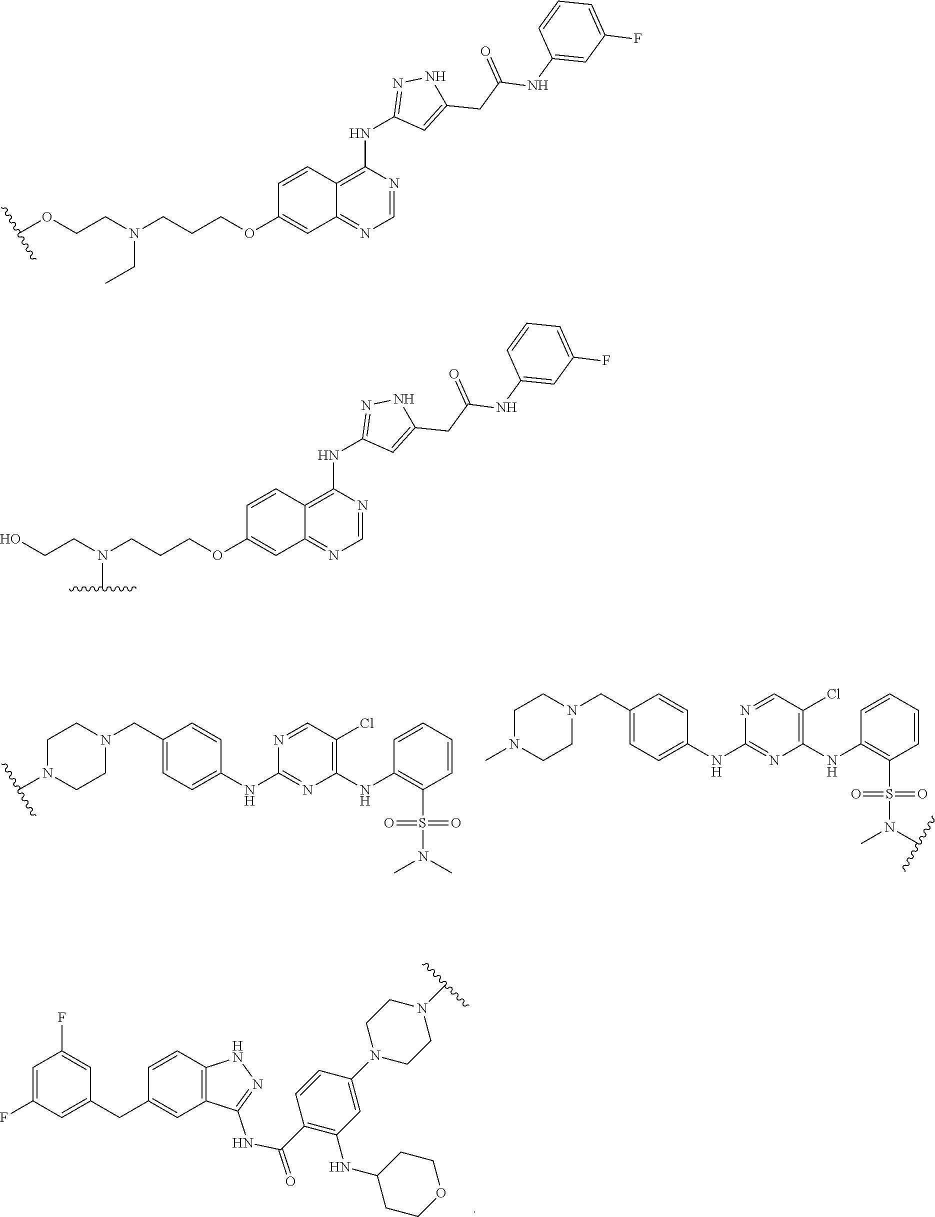

### I. Moiety for MEK

In certain embodiments, the EPL is a moiety that binds to and inhibits Mitogen-activated protein kinase kinase (MEK). Exemplary compounds that bind to and inhibit MEK are reported in the literature, including:

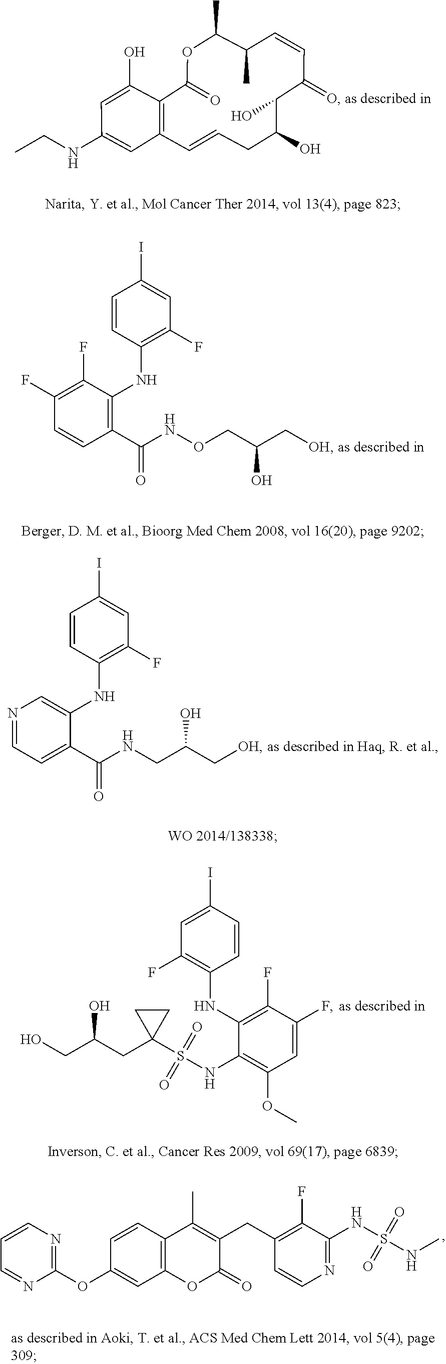

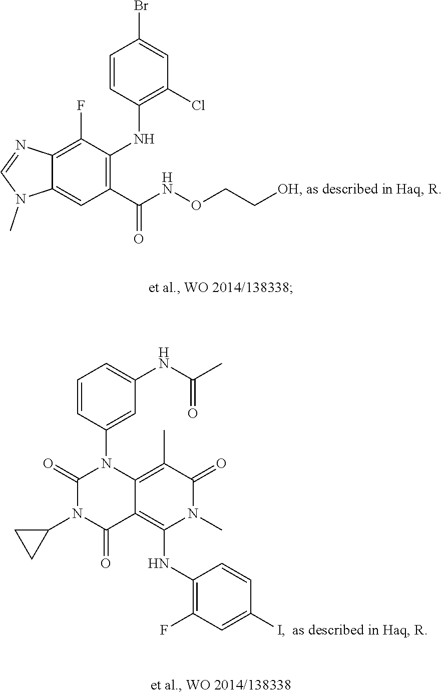

In certain embodiments, the EPL is a radical of one of the above compounds, which is attached to L through a modifiable oxygen, nitrogen, or carbon atom.

In certain embodiments, the EPL is a moiety that binds to and inhibits MEK1. In certain embodiments, the EPL is a moiety that binds to and inhibits MEK2. In certain embodiments, the EPL is a moiety that binds to and inhibits both MEK1 and MEK2. In certain embodiments, the EPL has the formula:

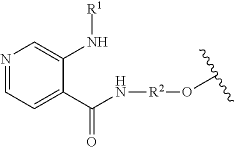

wherein:
   R¹ is phenyl or a 5-6 membered heteroaryl containing 1, 2, or 3 heteroatoms independently selected from nitrogen, oxygen, and sulfur; each of which is optionally substituted with 1, 2, or 3 substituents independently selected from halo, C₁-C₆ alkyl, C₁-C₆ haloalkyl, C₃-C₆ cycloalkyl, hydroxyl, C₁-C₆ alkoxy, or cyano; and
   R² is —(C₂₋₆ alkylene optionally substituted by one hydroxyl).

In certain embodiments, the EPL is one of the following:

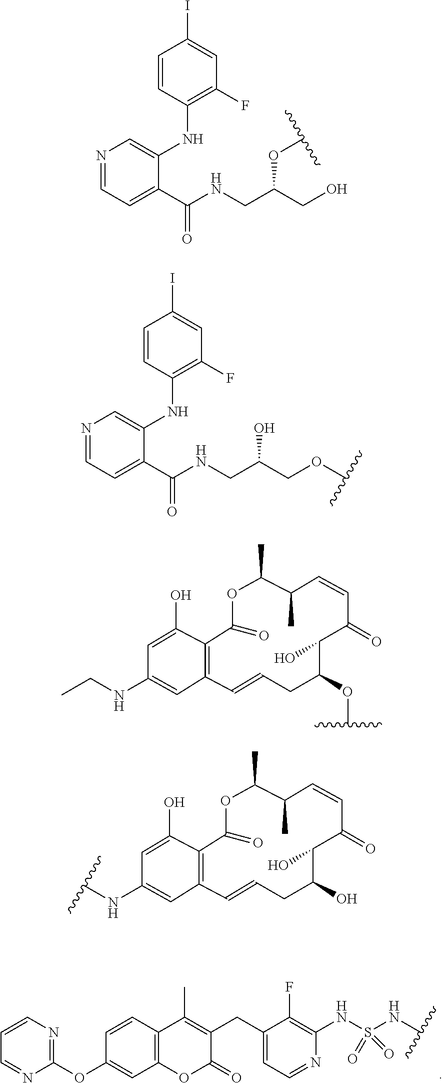

### 1. Moiety for Src

In certain embodiments, the EPL is a moiety that binds to and inhibits proto-oncogene tyrosine-protein kinase (Src). Exemplary compounds that bind to and inhibit Src are reported in the literature, including:

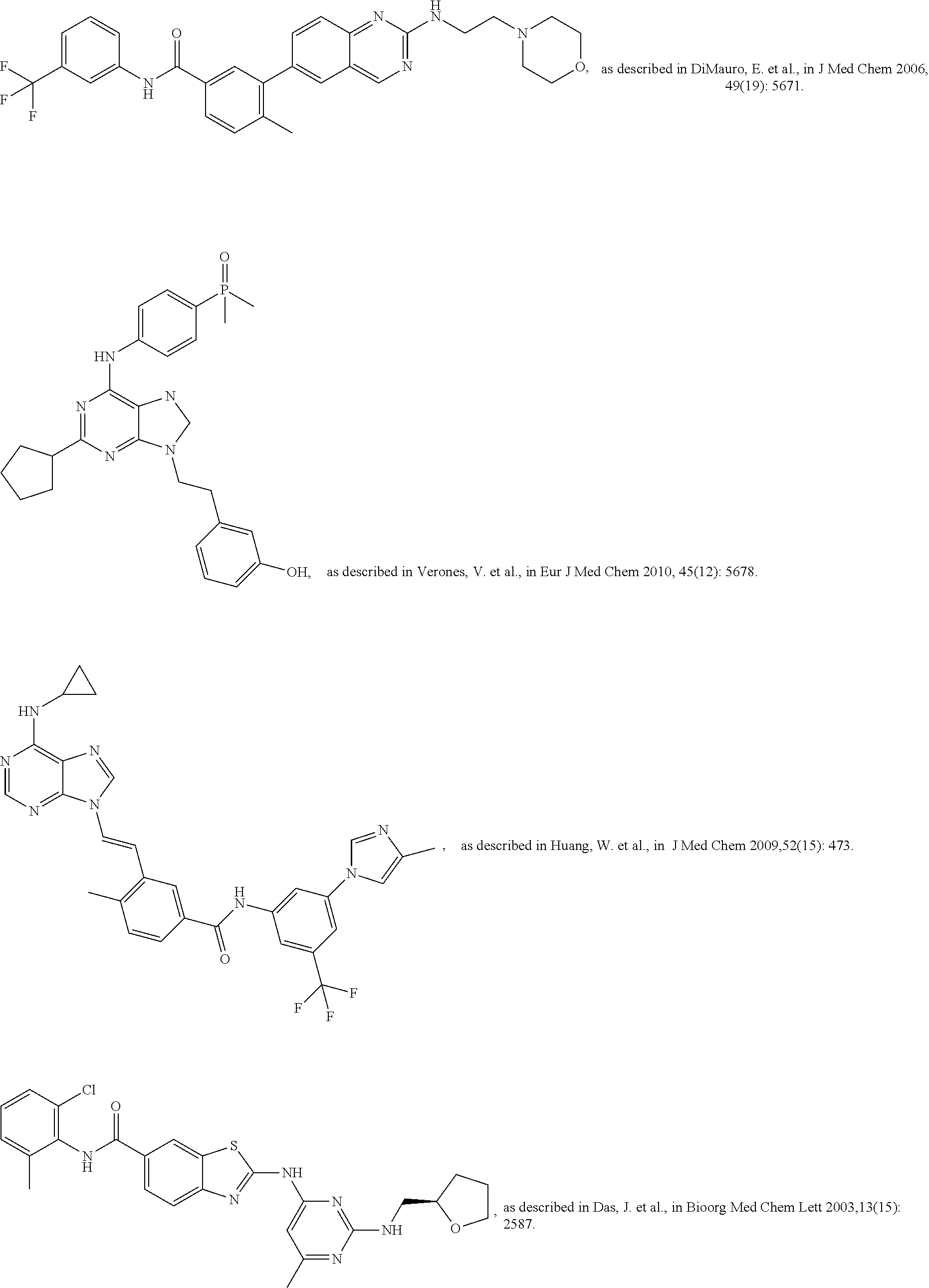 [SMILES: Cc1ccc(C(=O)Nc2cccc(C(F)(F)F)c2)cc1-c1ccc2nc(NCCN3CCOCC3)ncc2c1 ; CP(C)(=O)c1ccc(Nc2nc(C3CCCC3)nc3c2NCN3CCc2cccc(O)c2)cc1 ; Cc1cn(-c2cc(NC(=O)c3ccc(C)c(/C=C/n4cnc5c(NC6CC6)ncnc54)c3)cc(C(F)(F)F)c2)cn1 ; Cc1cc(Nc2nc3ccc(C(=O)Nc4c(C)cccc4Cl)cc3s2)nc(NC[C@H]2CCCO2)n1]

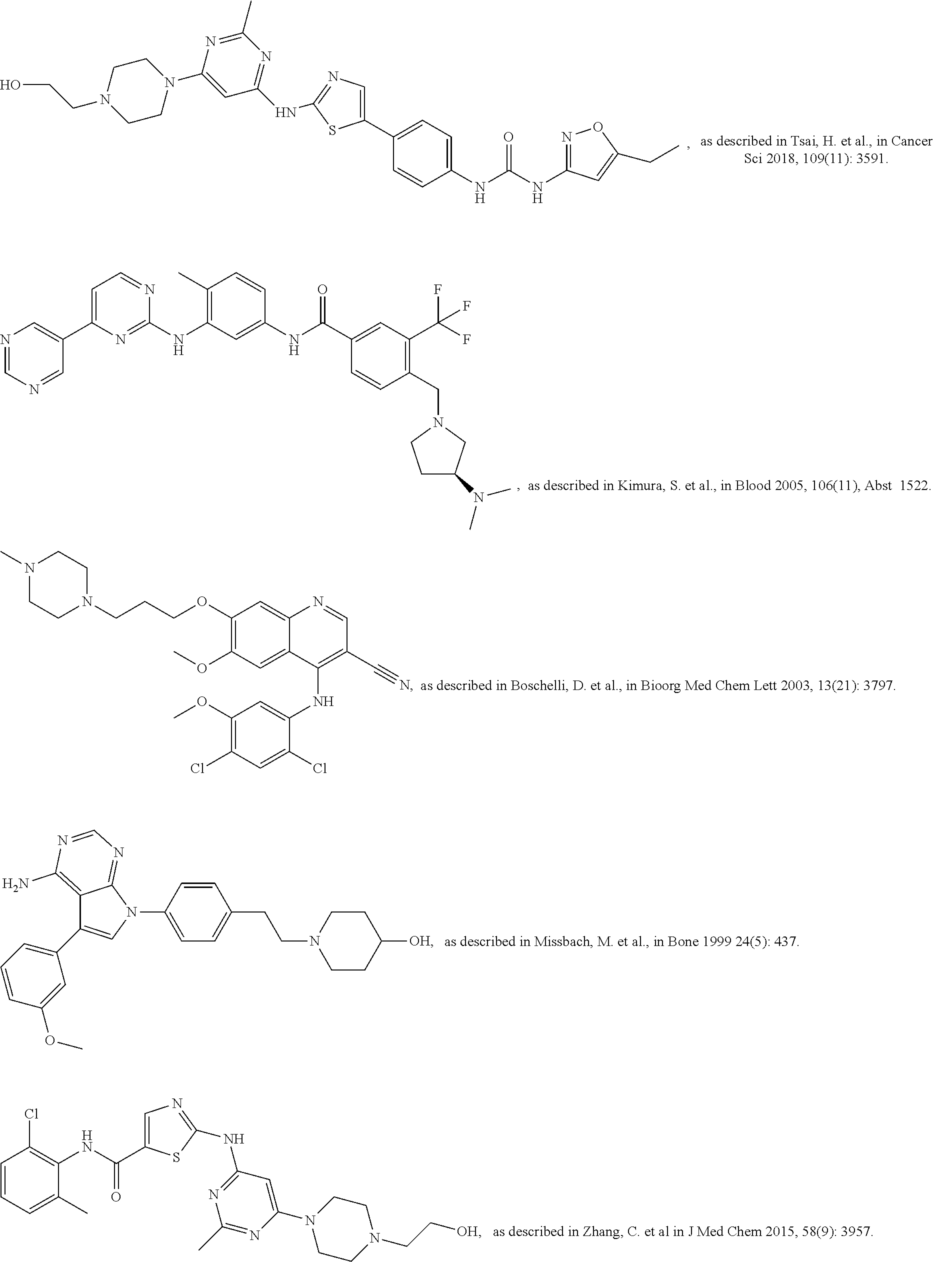

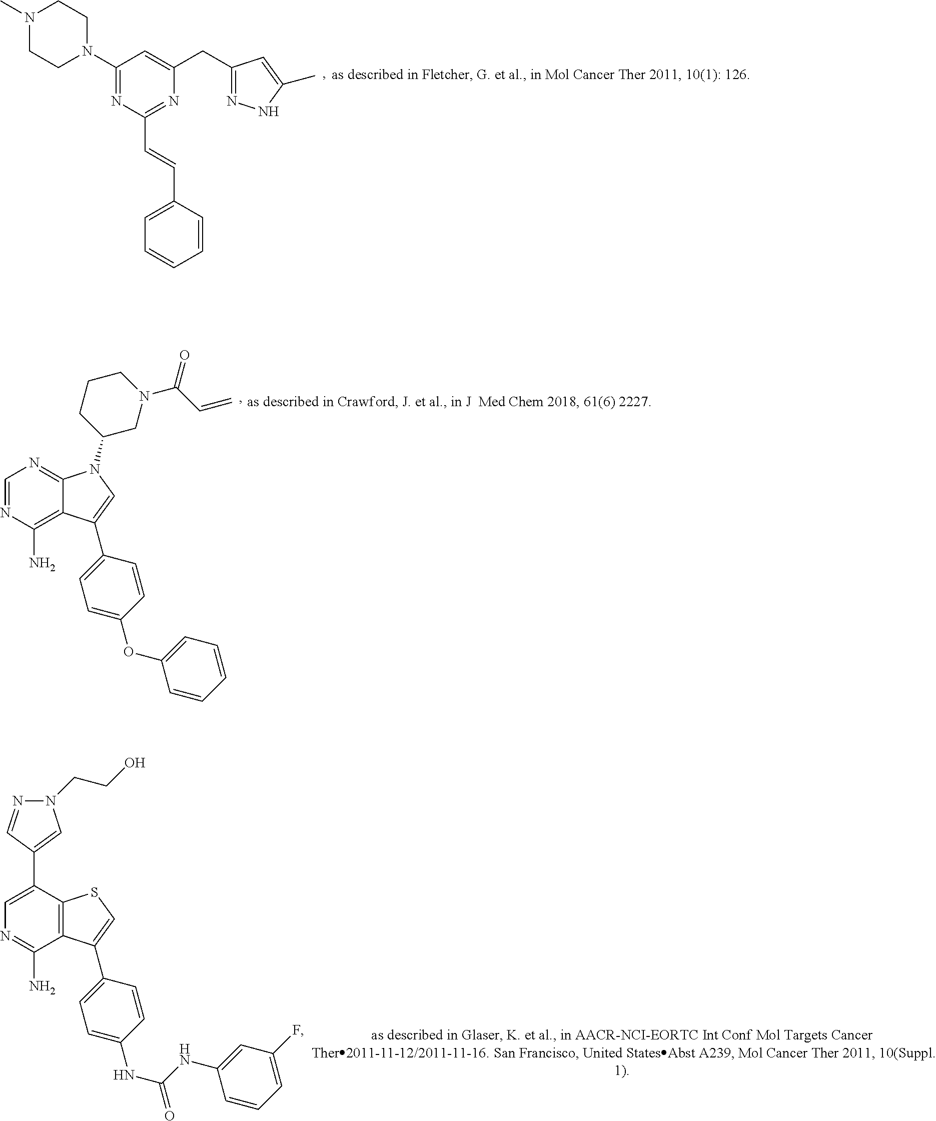

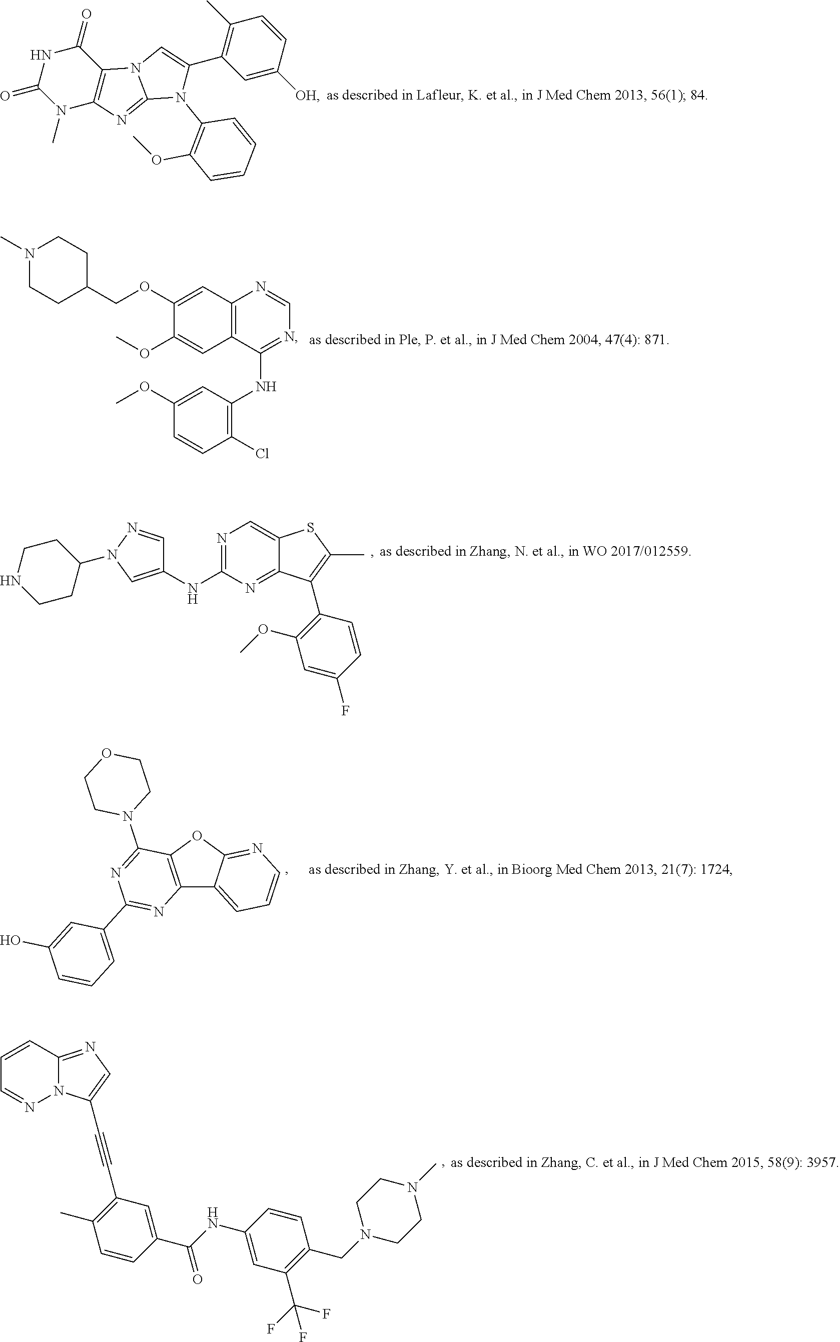 [SMILES: COc1ccccc1-n1c(-c2cc(O)ccc2C)cn2c3c(=O)[nH]c(=O)n(C)c3nc12 ; COc1ccc(Cl)c(Nc2ncnc3cc(OCC4CCN(C)CC4)c(OC)cc23)c1 ; COc1cc(F)ccc1-c1c(C)sc2cnc(Nc3cnn(C4CCNCC4)c3)nc12 ; Oc1cccc(-c2nc(N3CCOCC3)c3oc4ncccc4c3n2)c1 ; Cc1ccc(C(=O)Nc2ccc(CN3CCN(C)CC3)c(C(F)(F)F)c2)cc1C#Cc1cnc2cccnn12]

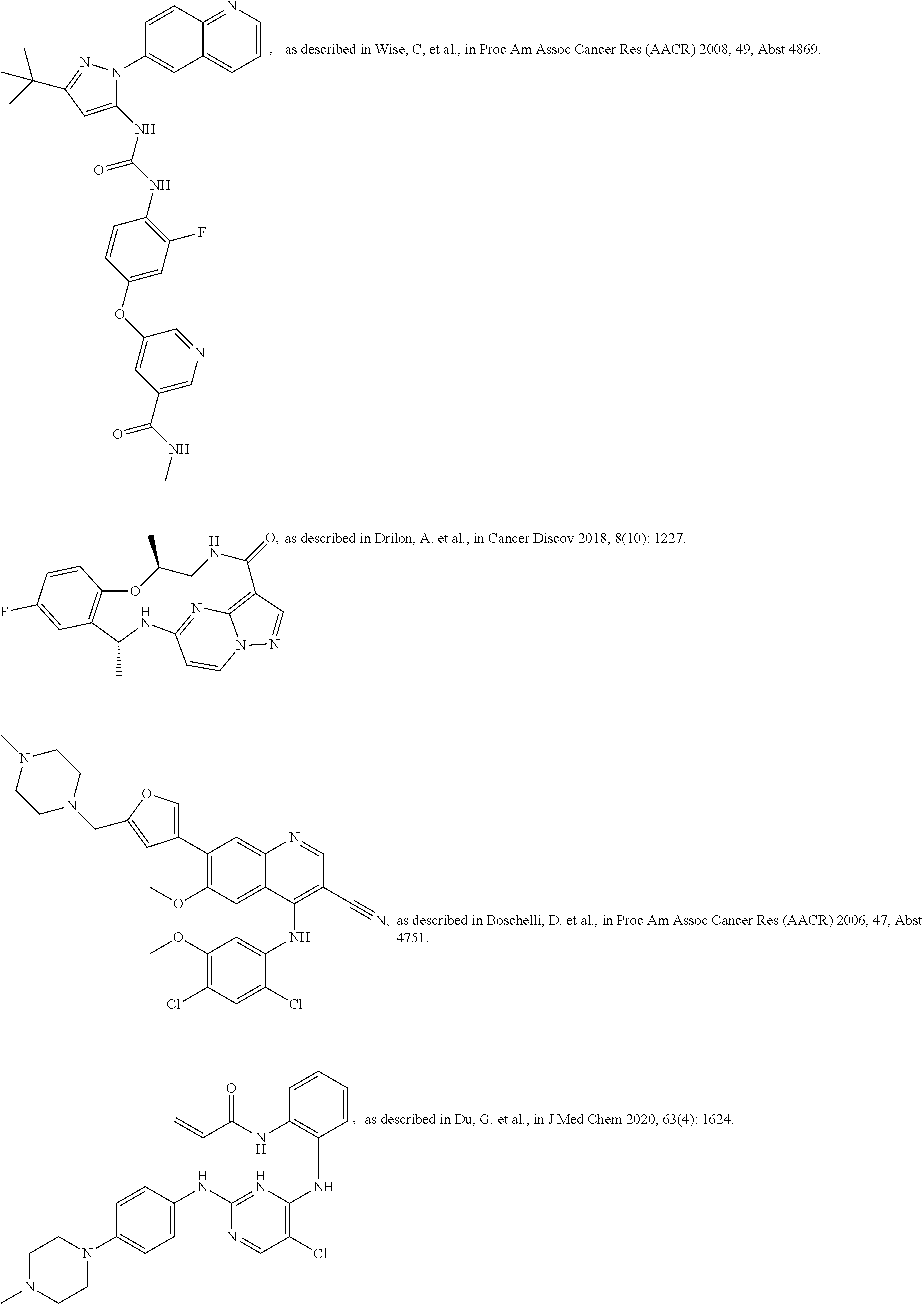

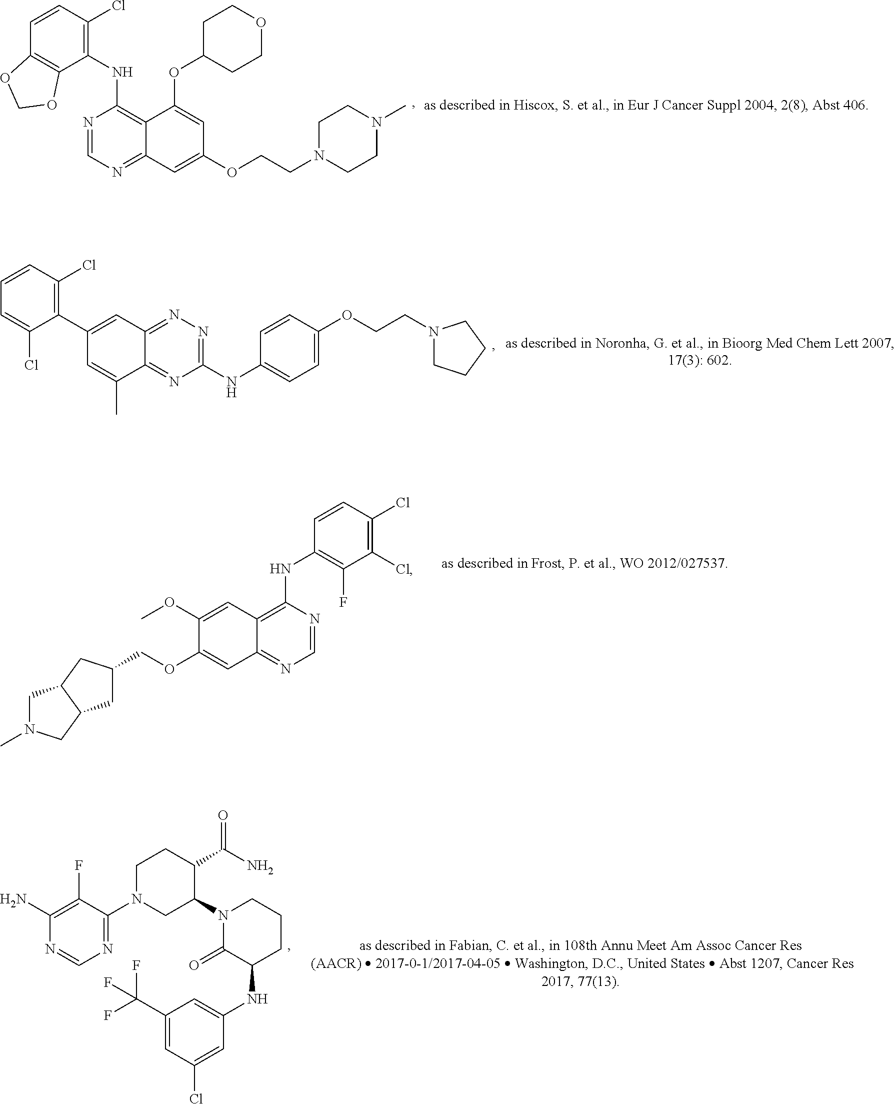

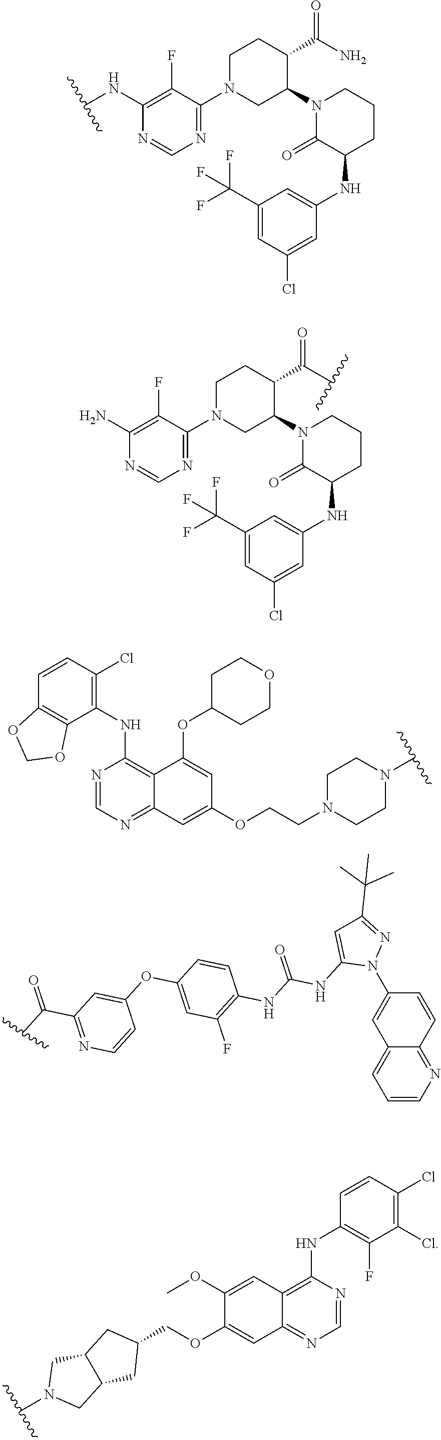

In certain embodiments, the EPL is a radical of one of the above compounds, which is attached to L through a modifiable oxygen, nitrogen, or carbon atom.

In certain embodiments, the EPL is one of the following:

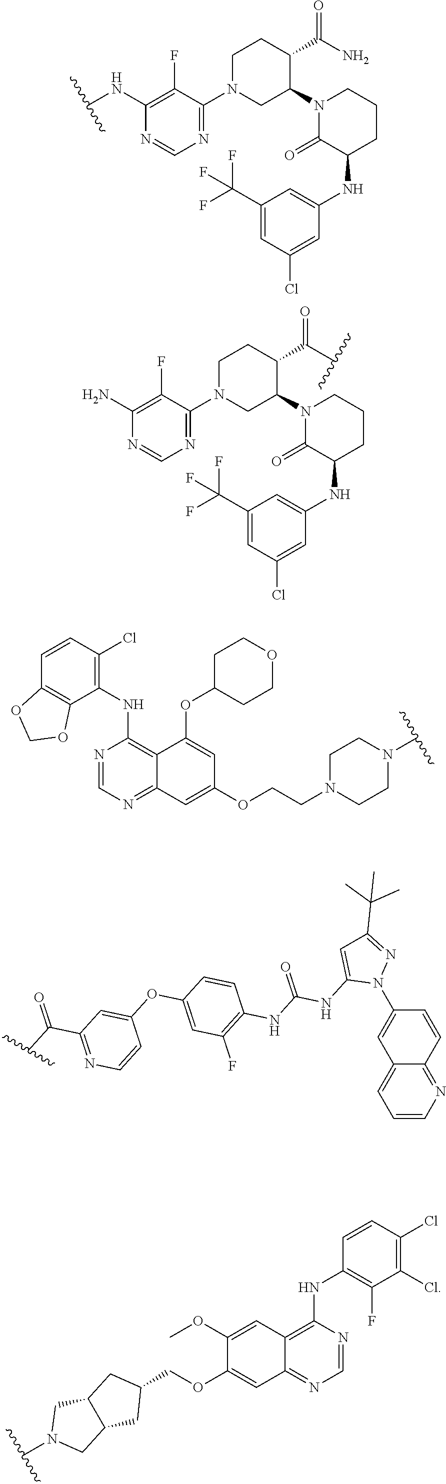

K. Moiety for c-KIT

In certain embodiments, the EPL is a moiety that binds to c-KIT. Exemplary compounds that bind to c-KIT are reported in the literature, including:

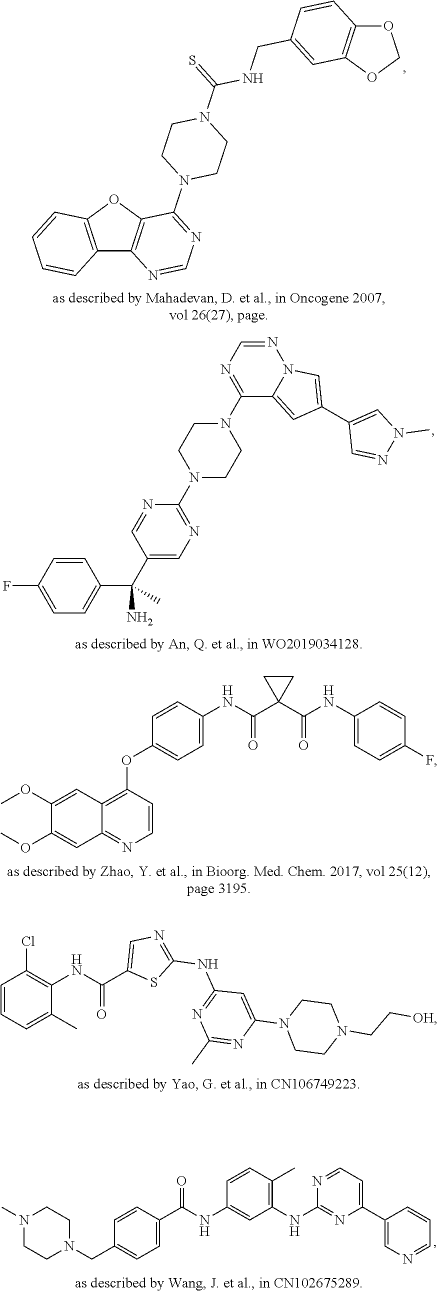

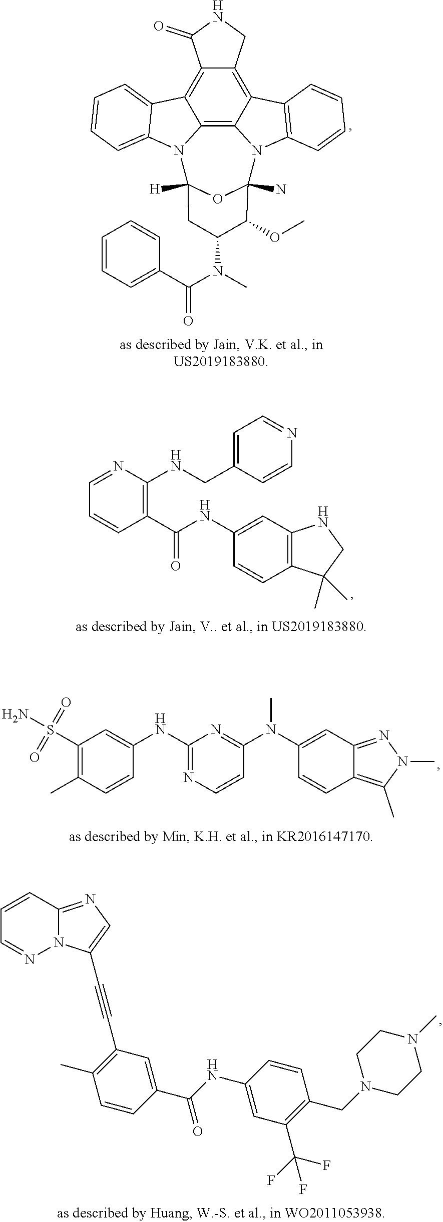 [SMILES: CO[C@@H]1[C@H](N(C)C(=O)c2ccccc2)C[C@H]2O[C@]1(N)n1c3ccccc3c3c4c(c5c6ccccc6n2c5c31)C(=O)NC4 ; CC1(C)CNc2cc(NC(=O)c3cccnc3NCc3ccncc3)ccc21 ; Cc1ccc(Nc2nccc(N(C)c3ccc4c(C)n(C)nc4c3)n2)cc1S(N)(=O)=O ; Cc1ccc(C(=O)Nc2ccc(CN3CCN(C)CC3)c(C(F)(F)F)c2)cc1C#Cc1cnc2cccnn12]

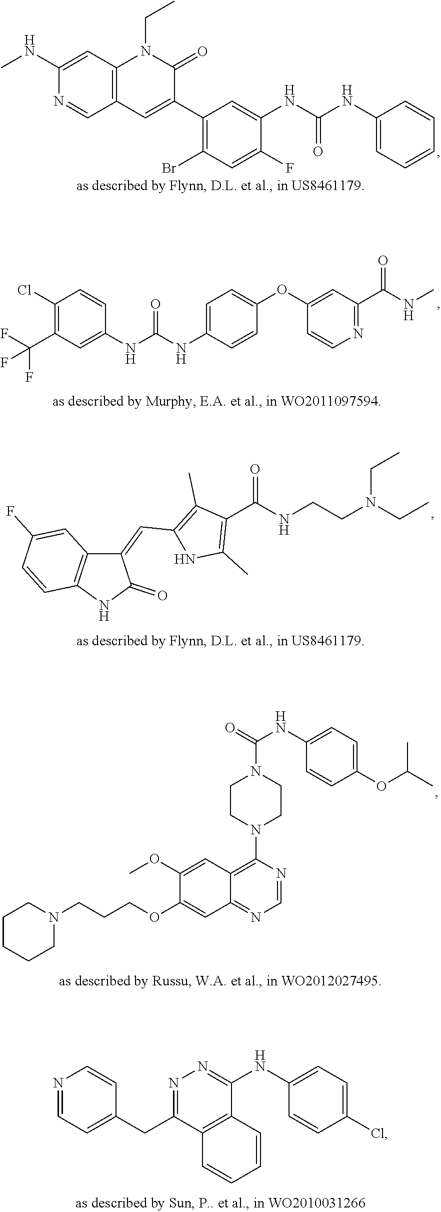 [SMILES: CCn1c(=O)c(-c2cc(NC(=O)Nc3ccccc3)c(F)cc2Br)cc2cnc(NC)cc21 ; CNC(=O)c1cc(Oc2ccc(NC(=O)Nc3ccc(Cl)c(C(F)(F)F)c3)cc2)ccn1 ; CCN(CC)CCNC(=O)c1c(C)[nH]c(/C=C2\C(=O)Nc3ccc(F)cc32)c1C ; COc1cc2c(N3CCN(C(=O)Nc4ccc(OC(C)C)cc4)CC3)ncnc2cc1OCCCN1CCCCC1 ; Clc1ccc(Nc2nnc(Cc3ccncc3)c3ccccc23)cc1]

In certain embodiments, the EPL is a radical of one of the above compounds, which is attached to L through a modifiable oxygen, nitrogen, or carbon atom.

In certain embodiments, the EPL is a radical of dasatinib, imatinib mesylate (STI571), sunitinib, regorafenib (BAY 73-4506), pazopanib HCl (GW786034 HCl), dovitinib (TKI-258), masitinib (AB1010), tivozanib (AV-951), motesanib diphosphate (AMG-706), amuvatinib (MP-470), levatinib (E7080), osi-930, Ki8751, telatinib, pozopanib, dovitinib (TKI-258), ripretinib (DCC-2618), sunitinib, Ki20227, avapritinib (BLU-285), AZD3229, AZD2932, regorafenib monohydrate, dovitinib (TKI258), pexidartinib (PLX3397), PDGFR inhibitor 1, or sitravatinib (MGCD516).

In certain embodiments, the EPL is one of the following:

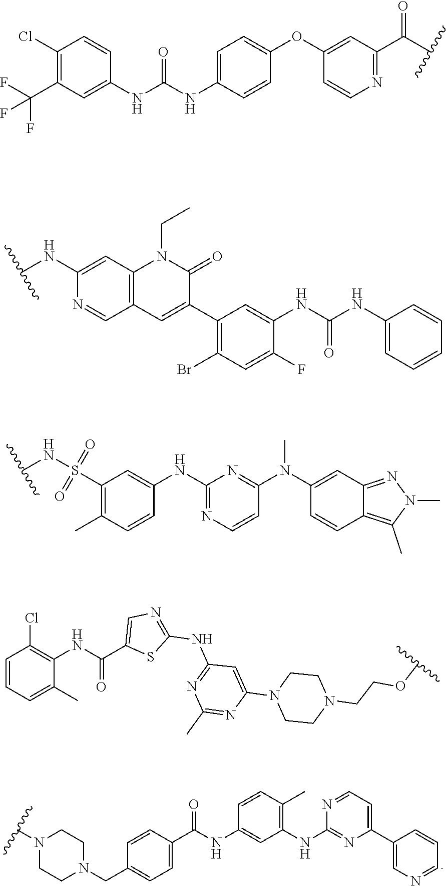 [SMILES: *C(=O)c1cc(Oc2ccc(NC(=O)Nc3ccc(Cl)c(C(F)(F)F)c3)cc2)ccn1 ; *Nc1cc2c(cn1)cc(-c1cc(NC(=O)Nc3ccccc3)c(F)cc1Br)c(=O)n2CC ; *NS(=O)(=O)c1cc(Nc2nccc(N(C)c3ccc4c(C)n(C)nc4c3)n2)ccc1C ; *OCCN1CCN(c2cc(Nc3ncc(C(=O)Nc4c(C)cccc4Cl)s3)nc(C)n2)CC1 ; *N1CCN(Cc2ccc(C(=O)Nc3ccc(C)c(Nc4nccc(-c5cccnc5)n4)c3)cc2)CC1]

### L. Moiety for KIF11

In certain embodiments, the EPL is a moiety that binds to Kinesin Family Member 11 (KIF11). Exemplary compounds that bind to KIF11 are reported in the literature, including:

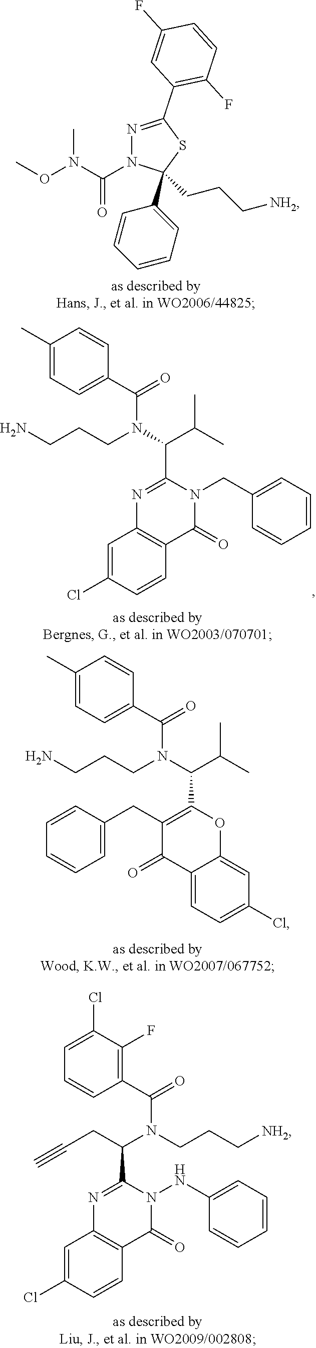 [SMILES: CON(C)C(=O)N1N=C(c2cc(F)ccc2F)S[C@@]1(CCCN)c1ccccc1 ; Cc1ccc(C(=O)N(CCCN)[C@@H](c2nc3cc(Cl)ccc3c(=O)n2Cc2ccccc2)C(C)C)cc1 ; Cc1ccc(C(=O)N(CCCN)[C@@H](c2oc3cc(Cl)ccc3c(=O)c2Cc2ccccc2)C(C)C)cc1 ; C#CC[C@H](c1nc2cc(Cl)ccc2c(=O)n1Nc1ccccc1)N(CCCN)C(=O)c1cccc(Cl)c1F]

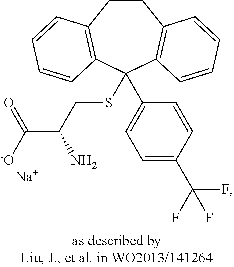

In certain embodiments, the EPL is a radical of one of the above compounds, which is attached to L through a modifiable oxygen, nitrogen, or carbon atom.

In certain embodiments, the EPL has the following formula:

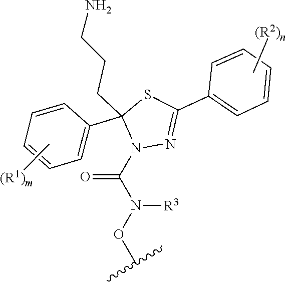

wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ is H, C₁-C₆ alkyl, C₁-C₆ haloalkyl, or C₃-C₆ cycloalkyl; and m and n each represent independently 0, 1, 2, or 3.

In certain embodiments, the EPL has the following formula:

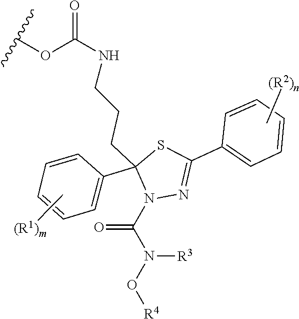

wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ and R⁴ each represent independently H, C₁-C₆ alkyl, C₁-C₆ haloalkyl, or C₃-C₆ cycloalkyl; and
m and n each represent independently 0, 1, 2, or 3.

In certain embodiments, the EPL is one of the following:

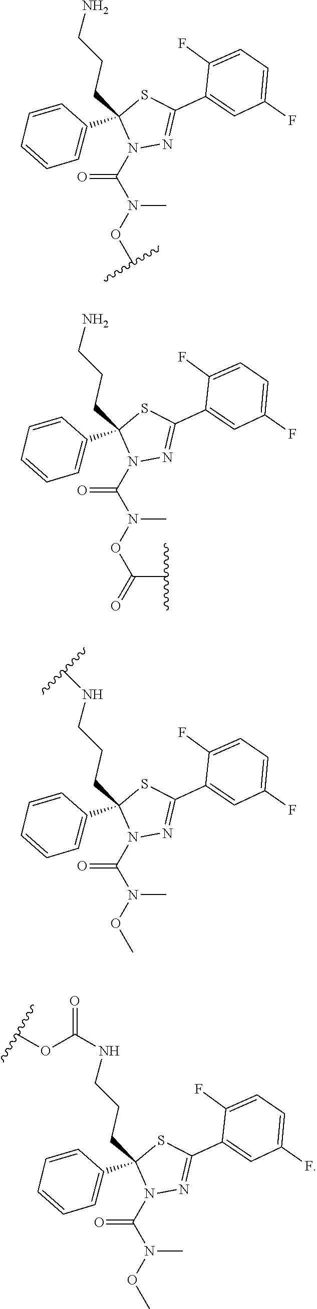 [SMILES: *ON(C)C(=O)N1N=C(c2cc(F)ccc2F)S[C@@]1(CCCN)c1ccccc1 ; *C(=O)ON(C)C(=O)N1N=C(c2cc(F)ccc2F)S[C@@]1(CCCN)c1ccccc1 ; *NCCC[C@@]1(c2ccccc2)SC(c2cc(F)ccc2F)=NN1C(=O)N(C)OC ; *OC(=O)NCCC[C@@]1(c2ccccc2)SC(c2cc(F)ccc2F)=NN1C(=O)N(C)OC]

In certain embodiments, the EPL is one of the following:

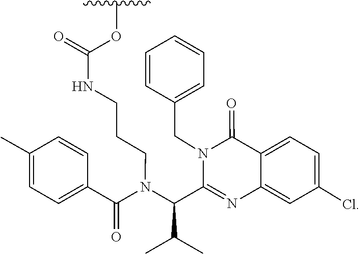 [SMILES: *OC(=O)NCCCN(C(=O)c1ccc(C)cc1)[C@@H](c1nc2cc(Cl)ccc2c(=O)n1Cc1ccccc1)C(C)C]

### M. Moiety for HSP90

In certain embodiments, the EPL is a moiety that binds to HSP90. Exemplary compounds that bind to HSP90 are reported in the literature, including:

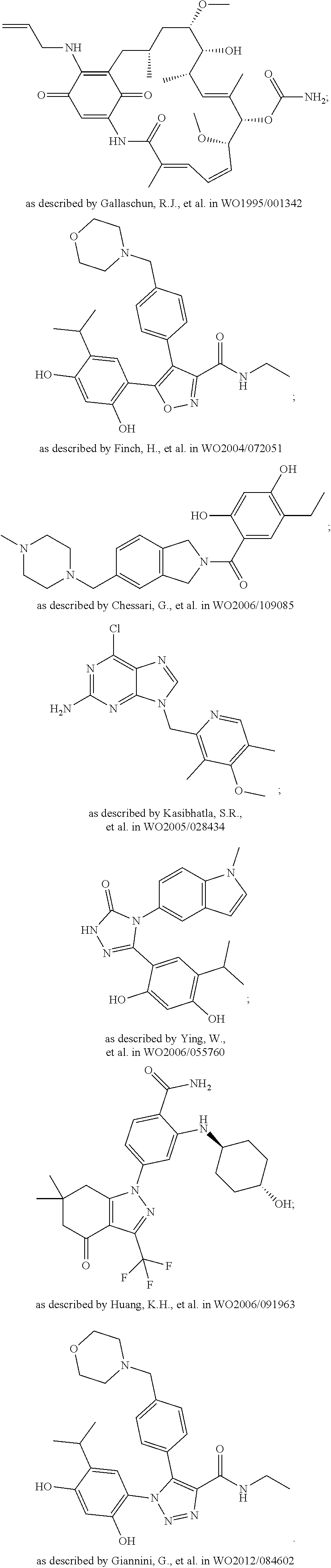

In certain embodiments, the EPL is a radical of one of the above compounds, which is attached to L through a modifiable oxygen, nitrogen, or carbon atom.

In certain embodiments, the EPL is one of the following:

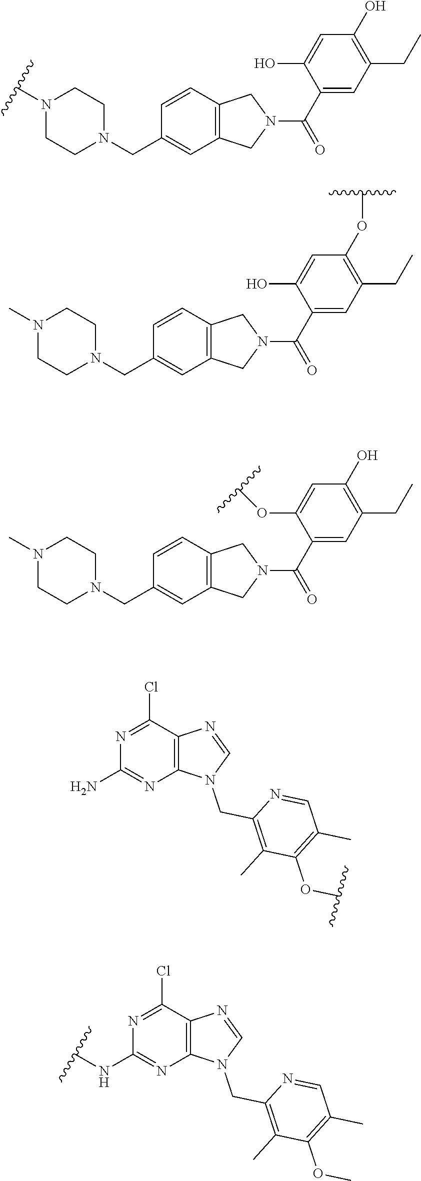

### N. Moiety for Tubulin

In certain embodiments, the EPL is a moiety that binds to tubulin. Exemplary compounds that bind to tubulin are reported in the literature, including:

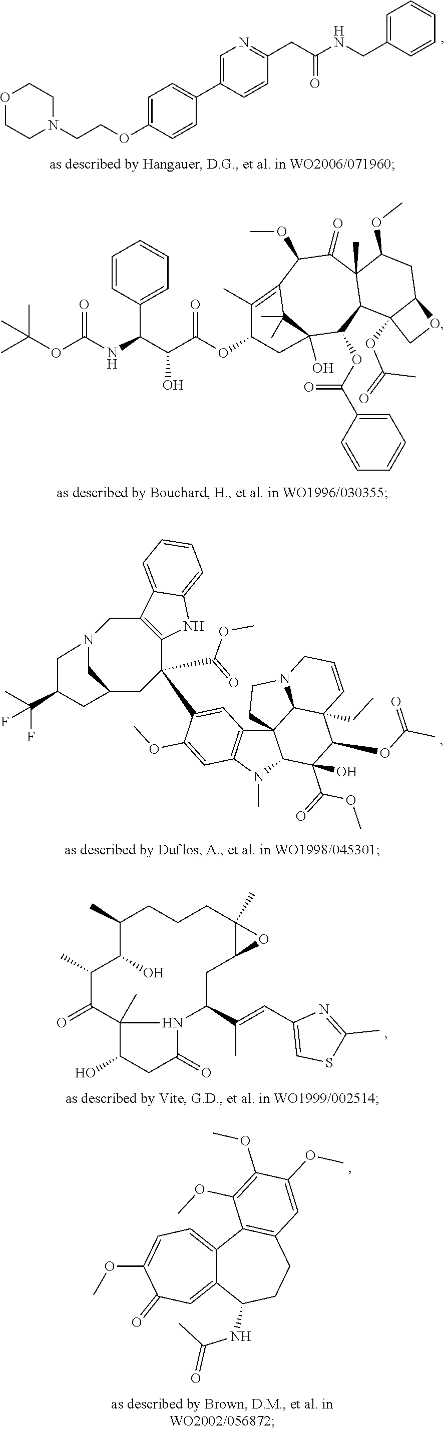

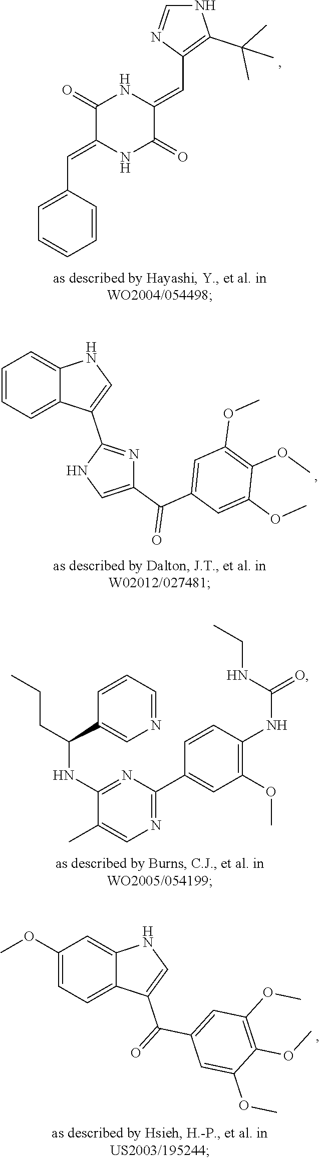

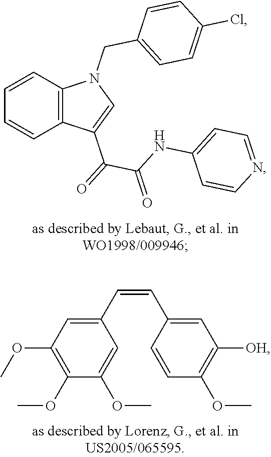

In certain embodiments, the EPL is a radical of one of the above compounds, which is attached to L through a modifiable oxygen, nitrogen, or carbon atom.

In certain embodiments, the EPL is one of the following:

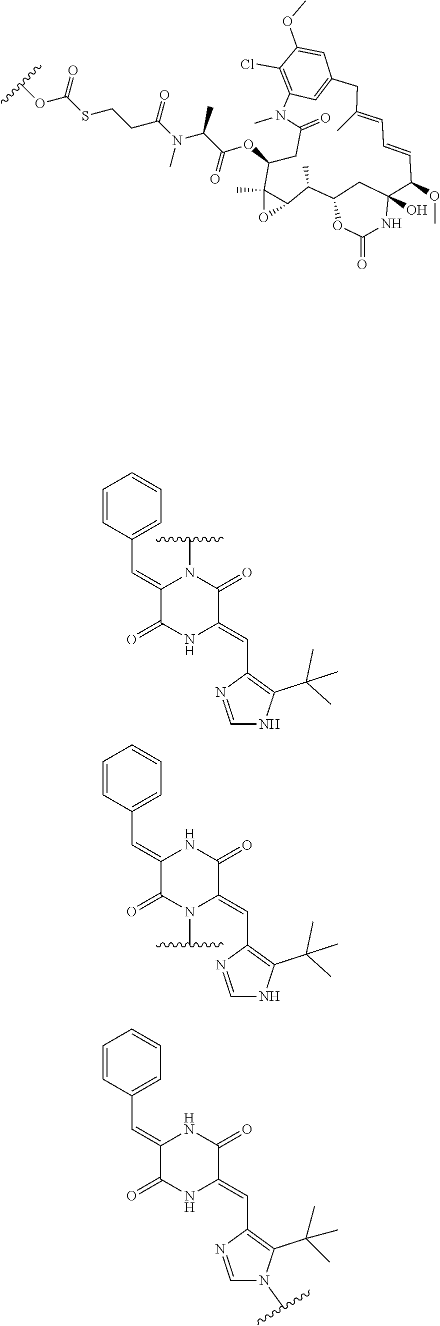 [SMILES: *OC(=O)SCCC(=O)N(C)[C@@H](C)C(=O)O[C@H]1CC(=O)N(C)c2cc(cc(OC)c2Cl)C/C(C)=C/C=C/[C@@H](OC)[C@@]2(O)C[C@H](OC(=O)N2)[C@@H](C)[C@@H]2O[C@@]12C ; *n1c(=O)/c(=C/c2nc[nH]c2C(C)(C)C)[nH]c(=O)/c1=C/c1ccccc1 ; *n1c(=O)/c(=C/c2ccccc2)[nH]c(=O)/c1=C/c1nc[nH]c1C(C)(C)C ; *n1cnc(/C=c2\[nH]c(=O)/c(=C/c3ccccc3)[nH]c2=O)c1C(C)(C)C]

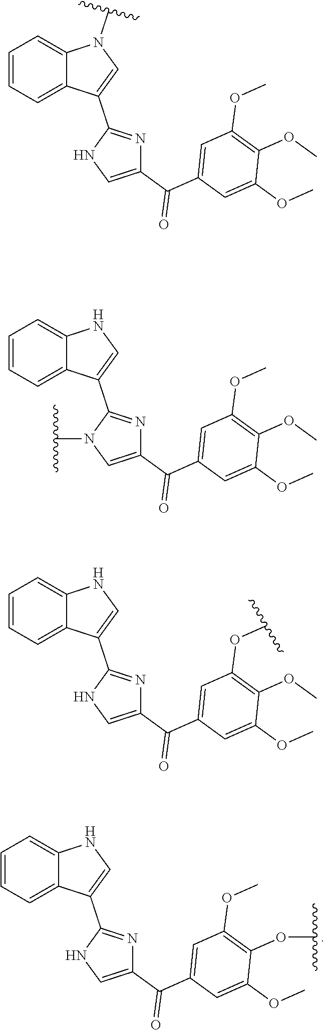 [SMILES: *n1cc(-c2nc(C(=O)c3cc(OC)c(OC)c(OC)c3)c[nH]2)c2ccccc21 ; *n1cc(C(=O)c2cc(OC)c(OC)c(OC)c2)nc1-c1c[nH]c2ccccc12 ; *Oc1cc(C(=O)c2c[nH]c(-c3c[nH]c4ccccc34)n2)cc(OC)c1OC ; *Oc1c(OC)cc(C(=O)c2c[nH]c(-c3c[nH]c4ccccc34)n2)cc1OC]

In certain embodiments, the EPL is one of the following:

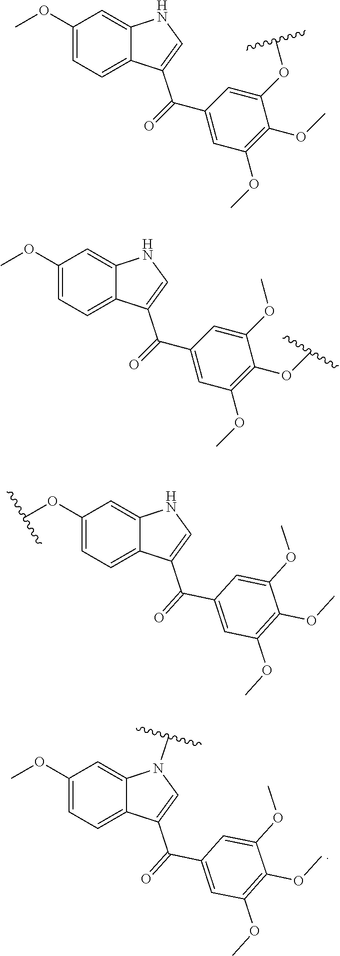

In certain embodiments, the EPL is one of the following:

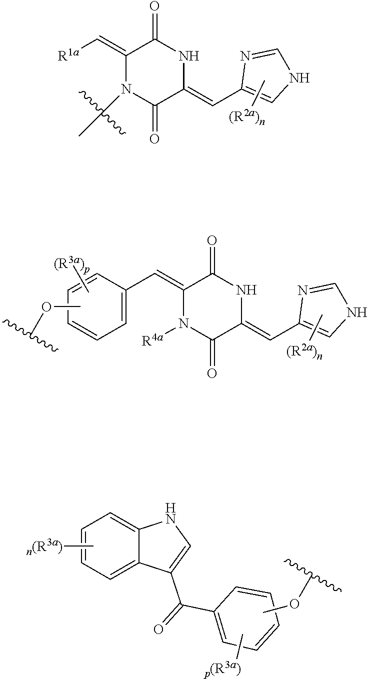

wherein:
R^{1a} is phenyl, C₃-C₆ cycloalkyl, or 5-6 membered heteroaryl containing 1 or 2 heteroatoms independently selected from oxygen, nitrogen, and sulfur; wherein the phenyl, cycloalkyl, and heteroaryl are substituted with 0, 1, 2, or 3 groups independently selected from C₁-C₆ alkyl, C₁-C₆ haloalkyl, C₃-C₆ cycloalkyl, halogen, hydroxyl, C₁-C₆ alkoxy, or —C(O)—(5-6 membered heteroaryl containing 1 or 2 heteroatoms independently selected from oxygen, nitrogen, and sulfur, wherein the heteroaryl is substituted with 0, 1, 2, or 3 groups independently selected from C₁-C₆ alkyl, C₁-C₆ haloalkyl, C₃-C₆ cycloalkyl, halogen, hydroxyl, or C₁-C₆ alkoxy);
R^{2a} each represent independently for each occurrence C₁-C₆ alkyl, C₁-C₆ haloalkyl, or C₃-C₆ cycloalkyl;
R^{3a} represents independently for each occurrence C₁-C₆ alkyl, C₁-C₆ haloalkyl, C₃-C₆ cycloalkyl, halogen, hydroxyl, or C₁-C₆ alkoxy;
R^{4a} is hydrogen, C₁-C₆ alkyl, or C₃-C₆ cycloalkyl;
n and p are independently 0, 1, or 2.

In certain embodiments, the EPL is one of the following:

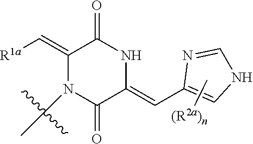

wherein:
R^{1a} is phenyl, C₃-C₆ cycloalkyl, or 5-6 membered heteroaryl containing 1 or 2 heteroatoms independently selected from oxygen, nitrogen, and sulfur; wherein the phenyl, cycloalkyl, and heteroaryl are substituted with 0, 1, 2, or 3 groups independently selected from C₁-C₆ alkyl, C₁-C₆ haloalkyl, C₃-C₆ cycloalkyl, halogen, hydroxyl, C₁-C₆ alkoxy, or —C(O)—(5-6 membered heteroaryl containing 1 or 2 heteroatoms independently selected from oxygen, nitrogen, and sulfur, wherein the heteroaryl is substituted with 0, 1, 2, or 3 groups independently selected from C₁-C₆ alkyl, C₁-C₆ haloalkyl, C₃-C₆ cycloalkyl, halogen, hydroxyl, or C₁-C₆ alkoxy);
R^{2a} each represent independently for each occurrence C₁-C₆ alkyl, C₁-C₆ haloalkyl, or C₃-C₆ cycloalkyl;
n is 0, 1, or 2.

In certain embodiments, R^{1a} is phenyl substituted with 0, 1, 2, or 3 groups independently selected from C₁-C₆ alkyl, C₁-C₆ haloalkyl, C₃-C₆ cycloalkyl, halogen, hydroxyl, or C₁-C₆ alkoxy. In certain embodiments, R^{1a} is phenyl.

In certain embodiments, R^{2a} is C₁-C₆ alkyl. In certain embodiments, R^{2a} is tert-butyl.

In certain embodiments, n is 0. In certain embodiments, n is 1. In certain embodiments, n is 2.

In certain embodiments, the EPL is one of the following:

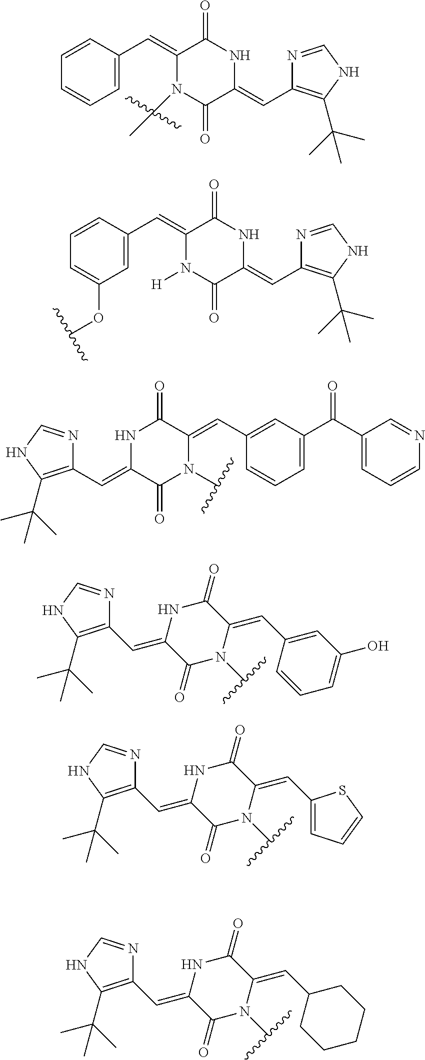

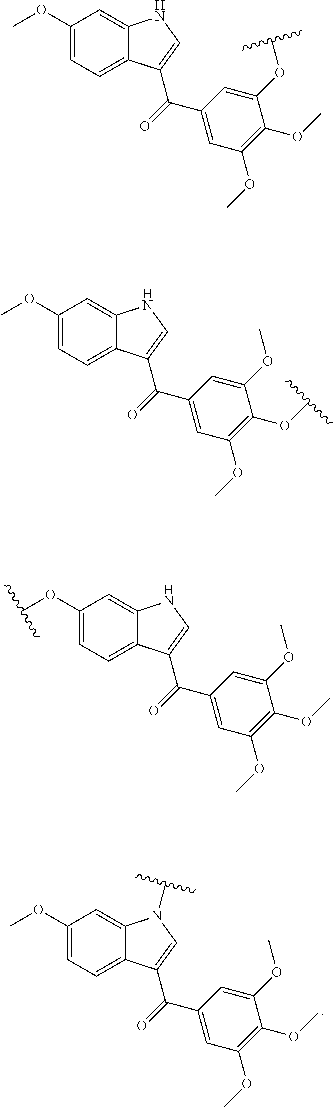

### O. Moiety for Proteasome

In certain embodiments, the EPL is a moiety that binds to and/or inhibits the proteasome. Exemplary compounds that bind to and/or inhibit the proteasome are reported in the literature, including:

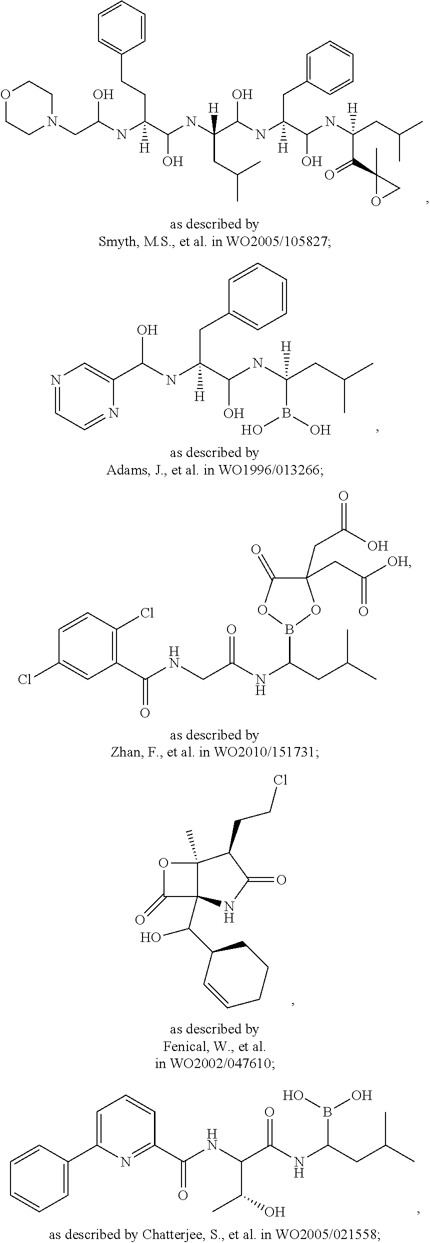 [SMILES: CC(C)C[C@H](NC(O)[C@H](Cc1ccccc1)NC(O)[C@H](CC(C)C)NC(O)[C@H](CCc1ccccc1)NC(O)CN1CCOCC1)C(=O)[C@@]1(C)CO1 ; CC(C)C[C@H](NC(O)[C@H](Cc1ccccc1)NC(O)c1cnccn1)B(O)O ; CC(C)CC(NC(=O)CNC(=O)c1cc(Cl)ccc1Cl)B1OC(=O)C(CC(=O)O)(CC(=O)O)O1 ; C[C@@]12OC(=O)[C@]1(C(O)[C@@H]1C=CCCC1)NC(=O)[C@@H]2CCCl ; CC(C)CC(NC(=O)C(NC(=O)c1cccc(-c2ccccc2)n1)[C@@H](C)O)B(O)O]

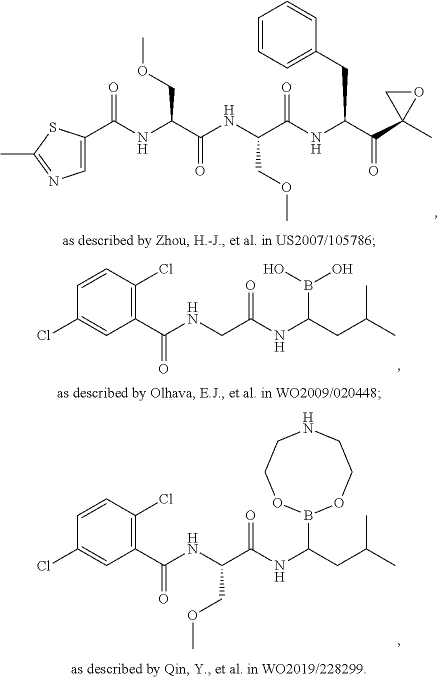

In certain embodiments, the EPL is a radical of one of the above compounds, which is attached to L through a modifiable oxygen, nitrogen, or carbon atom.

In certain embodiments, the EPL is one of the following:

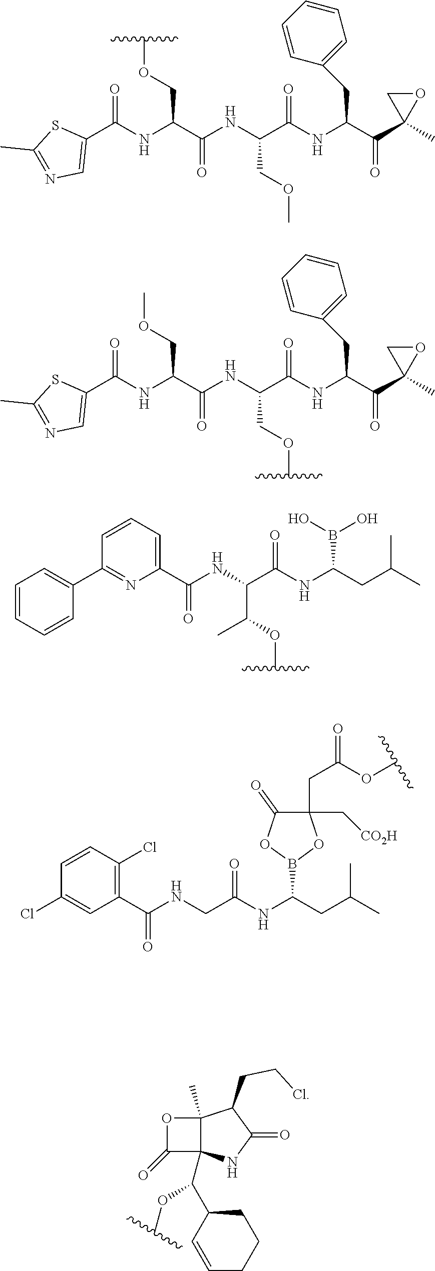

### P. Moiety for Topoisomerase

In certain embodiments, the EPL is a moiety that binds to topoisomerase.

In certain embodiments, the EPL is a moiety that binds to DNA Topoisomerase I (TOP1). Exemplary compounds that bind to TOP1 are reported in the literature, including:

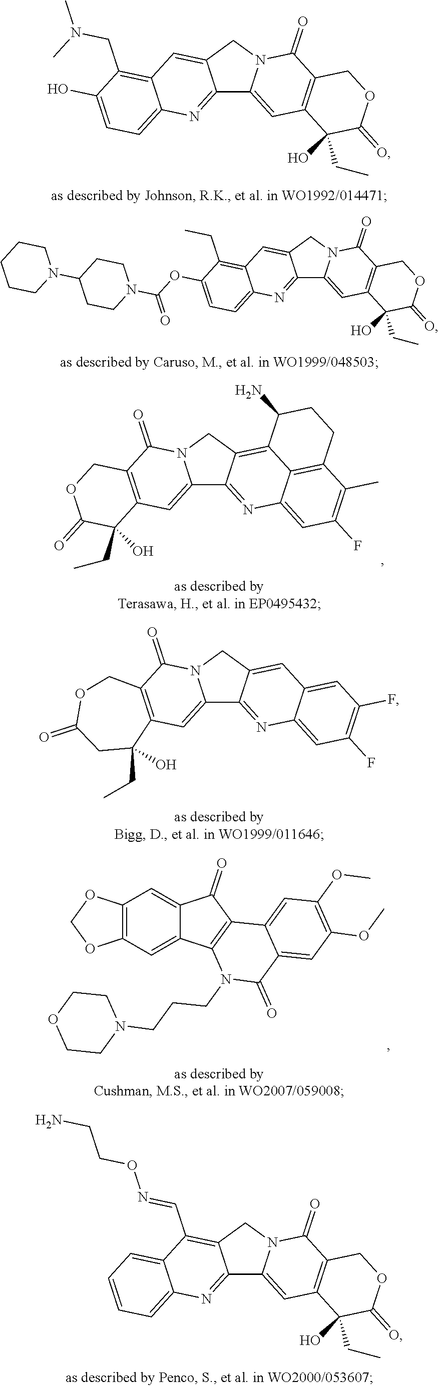

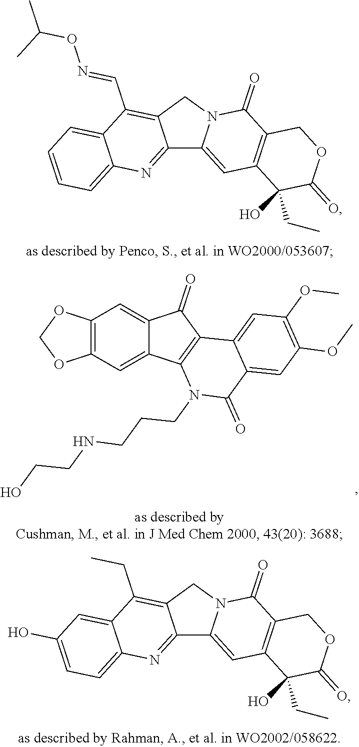 [SMILES: CC[C@@]1(O)C(=O)OCc2c1cc1n(c2=O)Cc2c-1nc1ccccc1c2/C=N/OC(C)C ; COc1cc2c3c(n(CCCNCCO)c(=O)c2cc1OC)-c1cc2c(cc1C3=O)OCO2 ; CCc1c2c(nc3ccc(O)cc13)-c1cc3c(c(=O)n1C2)COC(=O)[C@]3(O)CC]

In certain embodiments, the EPL is a radical of one of the above compounds, which is attached to L through a modifiable oxygen, nitrogen, or carbon atom.

In certain embodiments, the EPL is a moiety that bind to topoisomerase I (TOP1).

In certain embodiments, the EPL is one of the following:

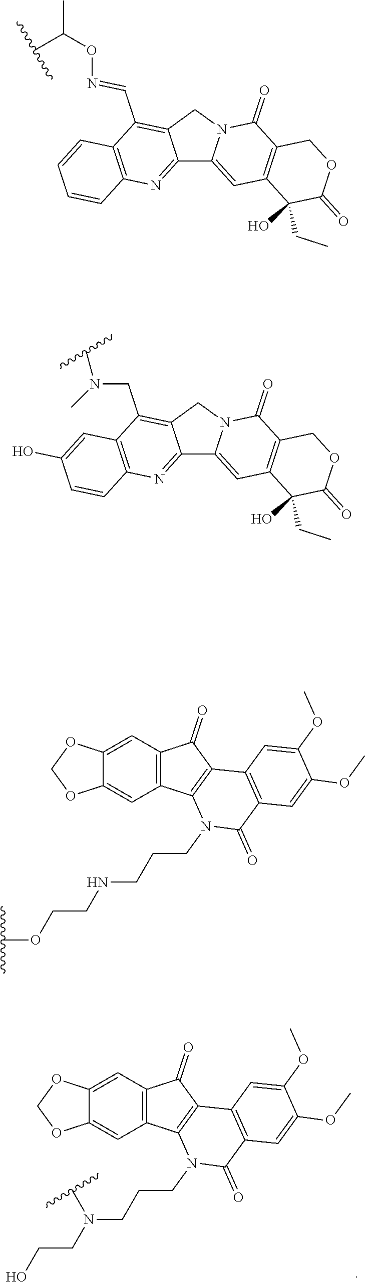 [SMILES: *C(C)O/N=C/c1c2c(nc3ccccc13)-c1cc3c(c(=O)n1C2)COC(=O)[C@]3(O)CC ; *N(C)Cc1c2c(nc3ccc(O)cc13)-c1cc3c(c(=O)n1C2)COC(=O)[C@]3(O)CC ; *OCCNCCCn1c2c(c3cc(OC)c(OC)cc3c1=O)C(=O)c1cc3c(cc1-2)OCO3 ; *N(CCO)CCCn1c2c(c3cc(OC)c(OC)cc3c1=O)C(=O)c1cc3c(cc1-2)OCO3]

In certain embodiments, the EPL is the following:

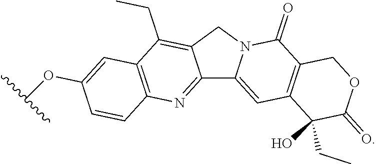 [SMILES: *Oc1ccc2nc3c(c(CC)c2c1)Cn1c-3cc2c(c1=O)COC(=O)[C@]2(O)CC]

### Q. Moiety for HDAC

In certain embodiments, the EPL is a moiety that binds to Histone Deacetylase (HDAC). Exemplary compounds that bind to HDAC are reported in the literature, including:

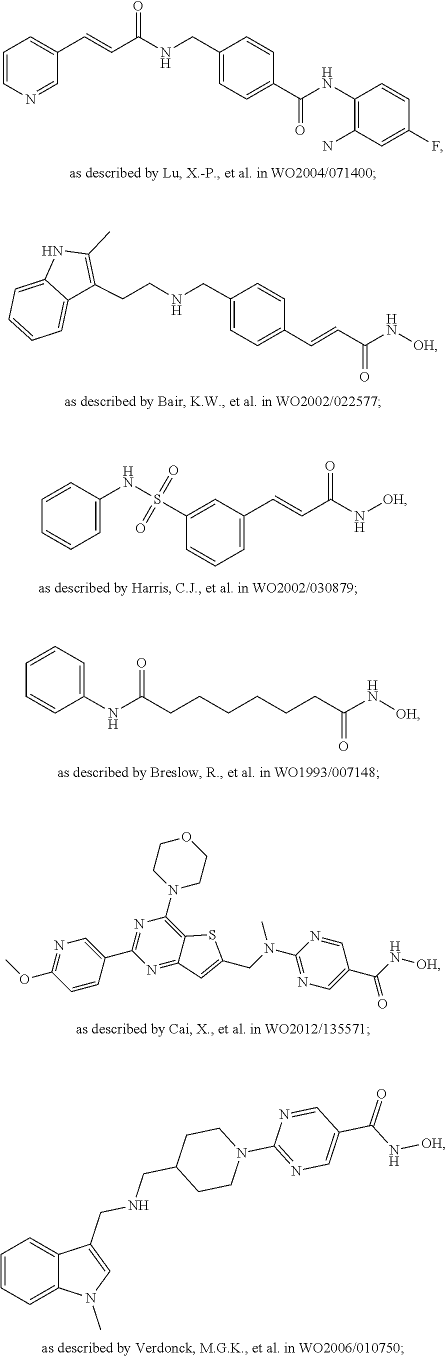

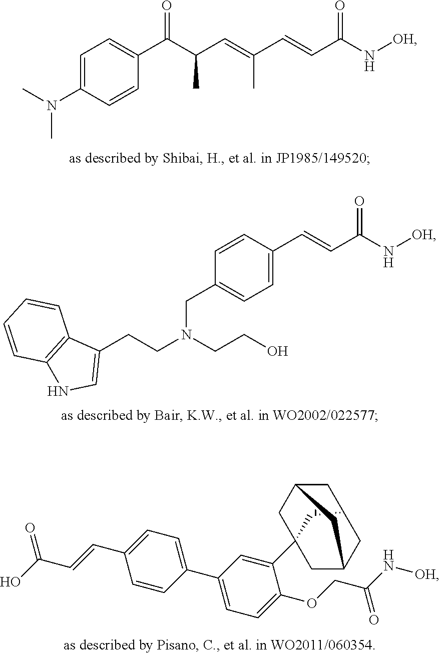 [SMILES: CC(/C=C/C(=O)NO)=C\[C@@H](C)C(=O)c1ccc(N(C)C)cc1 ; O=C(/C=C/c1ccc(CN(CCO)CCc2c[nH]c3ccccc23)cc1)NO ; O=C(O)/C=C/c1ccc(-c2ccc(OCC(=O)NO)c([C@]34C[C@H]5C[C@H](C[C@H](C5)C3)C4)c2)cc1]

In certain embodiments, the EPL is a radical of one of the above compounds, which is attached to L through a modifiable oxygen, nitrogen, or carbon atom.

In certain embodiments, the EPL is one of the following:

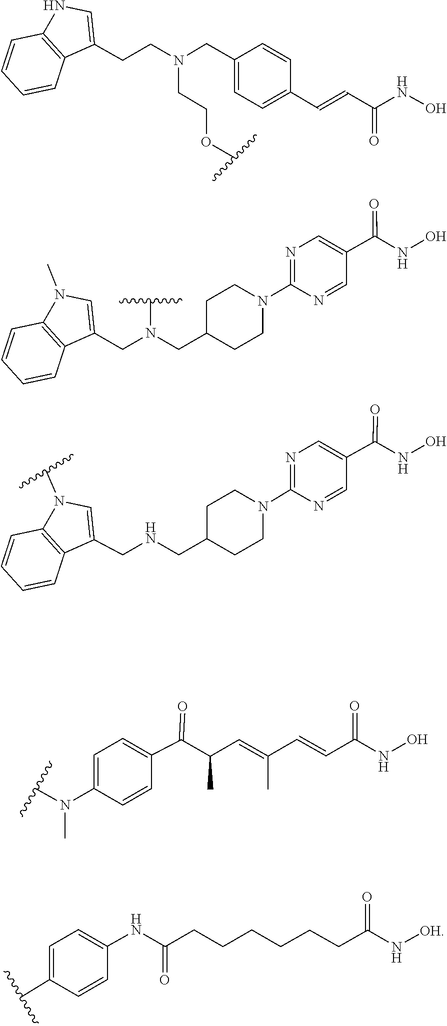 [SMILES: *OCCN(CCc1c[nH]c2ccccc12)Cc1ccc(/C=C/C(=O)NO)cc1 ; *N(Cc1cn(C)c2ccccc12)CC1CCN(c2ncc(C(=O)NO)cn2)CC1 ; *n1cc(CNCC2CCN(c3ncc(C(=O)NO)cn3)CC2)c2ccccc21 ; *N(C)c1ccc(C(=O)[C@H](C)/C=C(C)/C=C/C(=O)NO)cc1 ; *c1ccc(NC(=O)CCCCCCC(=O)NO)cc1]

In certain embodiments, EPL is selected from those depicted in the compounds in Table 3, below. In certain embodiments, EPL is selected from those depicted in the compounds in Table 1 or 1-A, below.

### Part B: Exemplary Further Description of TPL Component of Compounds of Formula I

Compounds of Formula I may be further characterized according to, for example, the identity of the TPL component. As generally described above, the TPL is a moiety that binds to a target protein selected from BTK, androgen receptor protein, or IDH1. In certain embodiments, TPL is a moiety that binds BTK. In certain embodiments, TPL is a moiety that binds androgen receptor protein. In certain embodiments, TPL is a moiety that binds IDH1. Exemplary moieties for the TPL component are described in more detail below.

### Moiety for BTK

In certain embodiments, the TPL is a moiety that binds to Bruton's tyrosine kinase (BTK). Exemplary compounds that bind to BTK are reported in the literature, such as ibrutinib and zanubrutinib. A radical of such compounds reported in the literature that bind BTK are amenable for use in the present invention.

In certain embodiments, the TPL is one of the following:

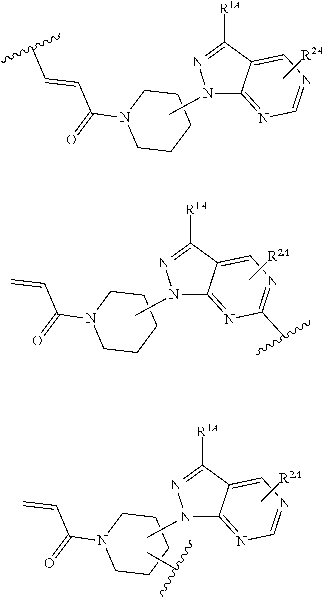

wherein:
R^{1A} is -(phenyl optionally substituted with 1, 2, 3, or 5 substituents independently selected from halo, hydroxyl, C₁₋₄ alkyl, and C₁₋₄ alkoxyl)-O-(phenyl optionally substituted with 1, 2, 3, or 5 substituents independently selected from halo, hydroxyl, C₁₋₄ alkyl, and C₁₋₄ alkoxyl);
R^{2A} is hydrogen, halo, hydroxyl, C₁₋₄ alkyl, C₁₋₄ alkoxyl, or —N(R^{5A})(R^{6A}); and
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to same nitrogen atom are taken together with the nitrogen atom to which they are attached to form a 3-7 membered heterocyclic ring.

In certain embodiments, the TPL is one of the following:

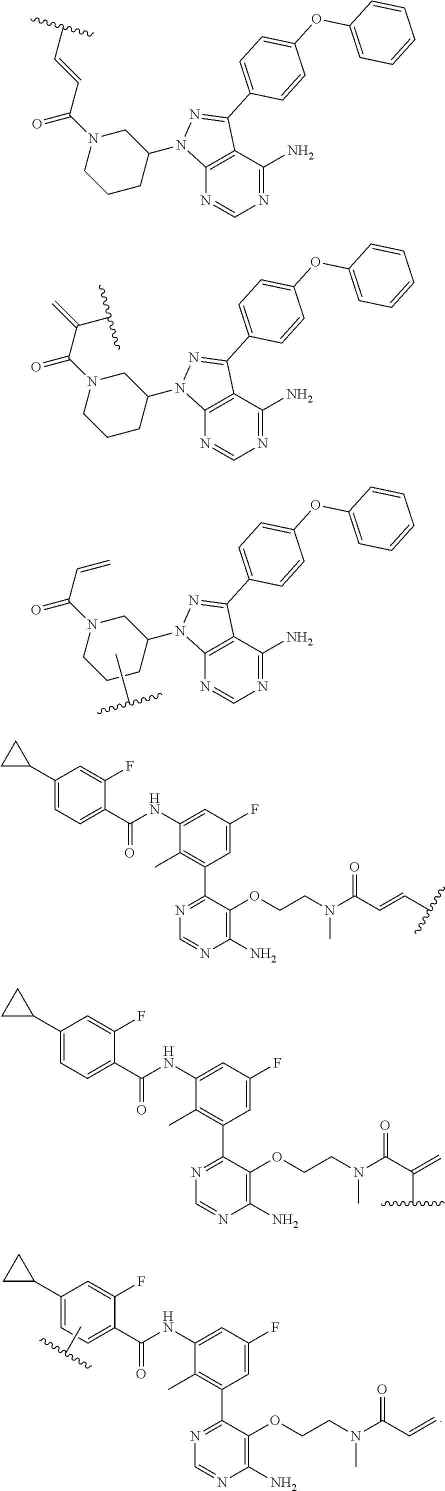

In certain embodiments, the TPL is

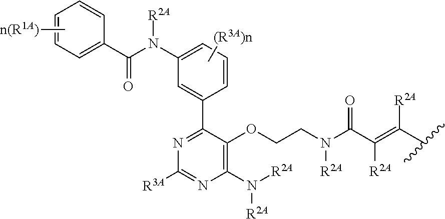

wherein:
R^{1A} and R^{3A} each represent independently for each occurrence hydrogen, halo, hydroxyl, C₁₋₄ alkyl, C₁₋₄ alkoxyl, or C₃₋₆ cycloalkyl;
R^{2A} is hydrogen, C₁₋₆ alkyl, or C₃₋₆ cycloalkyl; and
n and m are independently 1 or 2.

In certain embodiments, R²⁴ represents independently for each occurrence hydrogen, C₁₋₆ alkyl, or C₃₋₆ cycloalkyl.

In certain embodiments, TPL is one of the following:

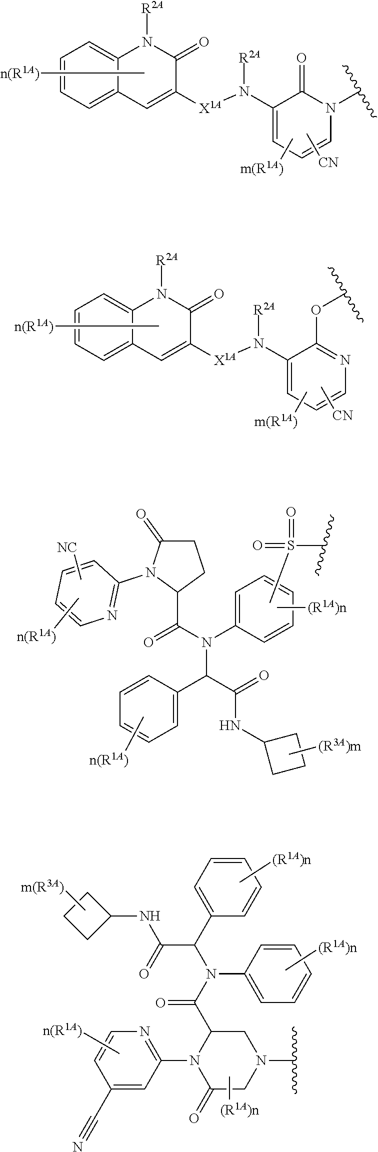

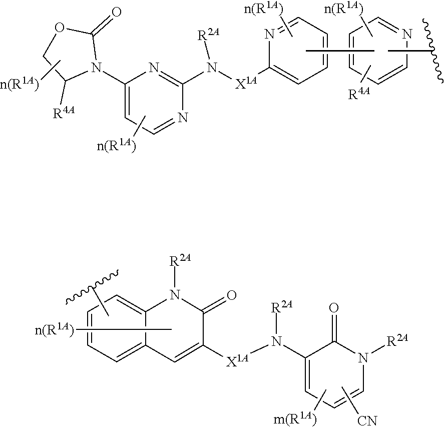

wherein:
R^{1A} and R^{3A} each represent independently for each occurrence hydrogen, halo, hydroxyl, C₁₋₄ alkyl, C₁₋₄ alkoxyl, or C₃₋₆ cycloalkyl;
R^{2A} is hydrogen, C₁₋₆ alkyl, or C₃₋₆ cycloalkyl;
R^{4A} is C₁₋₄ haloalkyl;
X^{1A} is C₁₋₆ alkylene; and n and m each represent independently for each occurrence 1 or 2.

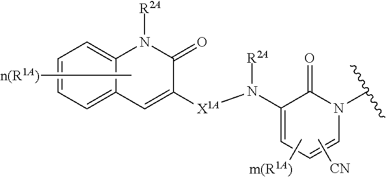

In certain embodiments, TPL is wherein:
R^{1A} represents independently for each occurrence hydrogen, halo, hydroxyl, C₁₋₄ alkyl, C₁₋₄ alkoxyl, or C₃₋₆ cycloalkyl;
R^{2A} is hydrogen, C₁₋₆ alkyl, or C₃₋₆ cycloalkyl;
X^{1A} is C₁₋₆ alkylene; and
n and m are independently 1 or 2.

In certain embodiments, TPL is

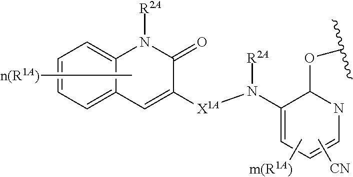

wherein:
R^{1A} and R^{3A} each represent independently for each occurrence hydrogen, halo, hydroxyl, C₁₋₄ alkyl, C₁₋₄ alkoxyl, or C₃₋₆ cycloalkyl;
R^{2A} is hydrogen, C₁₋₆ alkyl, or C₃₋₆ cycloalkyl;
X^{1A} is C₁₋₆ alkylene; and
n and m are independently 1 or 2.

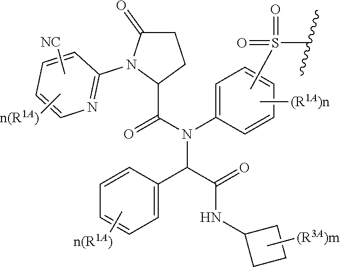

In certain embodiments, TPL is wherein:
R^{1A} and R^{3A} each represent independently for each occurrence hydrogen, halo, hydroxyl, C₁₋₄ alkyl, C₁₋₄ alkoxyl, or C₃₋₆ cycloalkyl;
R^{2A} is hydrogen, C₁₋₆ alkyl, or C₃₋₆ cycloalkyl; and
n and m each represent independently for each occurrence 1 or 2.

In certain embodiments, TPL is one of the following:

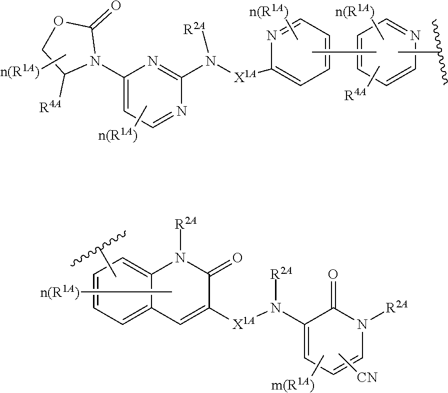

wherein:
R^{1A} and R^{3A} each represent independently for each occurrence hydrogen, halo, hydroxyl, C₁₋₄ alkyl, C₁₋₄ alkoxyl, or C₃₋₆ cycloalkyl;
R^{2A} is hydrogen, C₁₋₆ alkyl, or C₃₋₆ cycloalkyl;
R^{4A} is C₁₋₄ haloalkyl;
X^{1A} is C₁₋₆ alkylene; and
n and m each represent independently for each occurrence 1 or 2.

In certain embodiments, the TPL is one of the following:

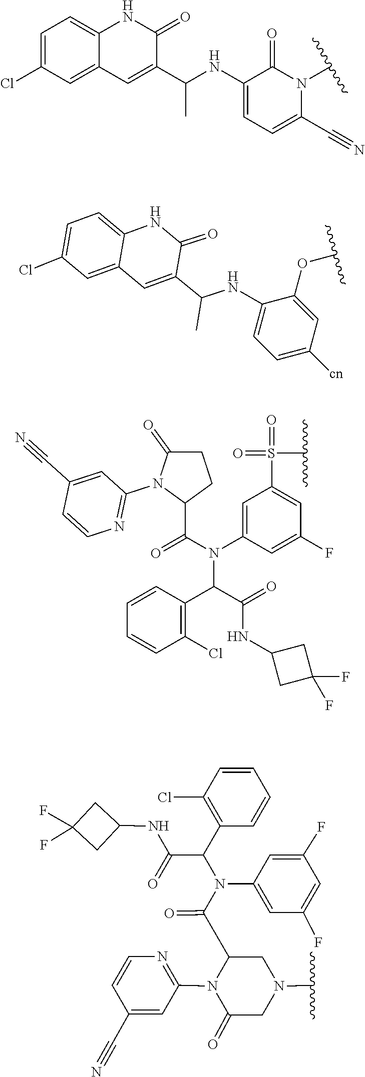

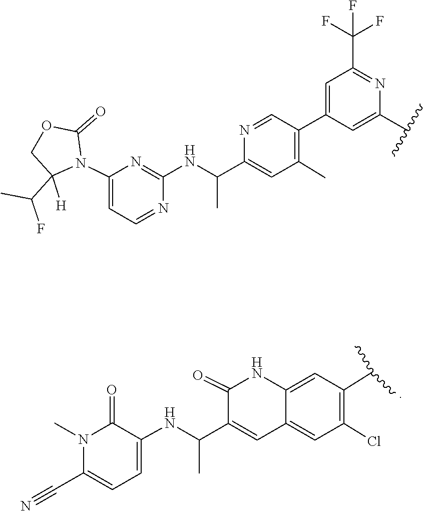 [SMILES: *c1cc(-c2cnc(C(C)Nc3nccc(N4C(=O)OCC4C(C)F)n3)cc2C)cc(C(F)(F)F)n1 ; *c1cc2[nH]c(=O)c(C(C)Nc3ccc(C#N)n(C)c3=O)cc2cc1Cl]

In certain embodiments, the TPL is

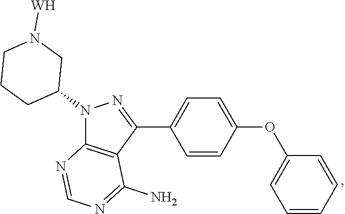

wherein WH is a group that reacts with BTK to form a covalent linkage. In certain embodiments, the TPL is

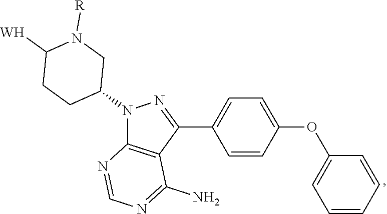

wherein WH is a group that reacts with BTK to form a covalent linkage, and R is H, alkyl, or acyl.

In certain embodiments, the TPL is one of the following:

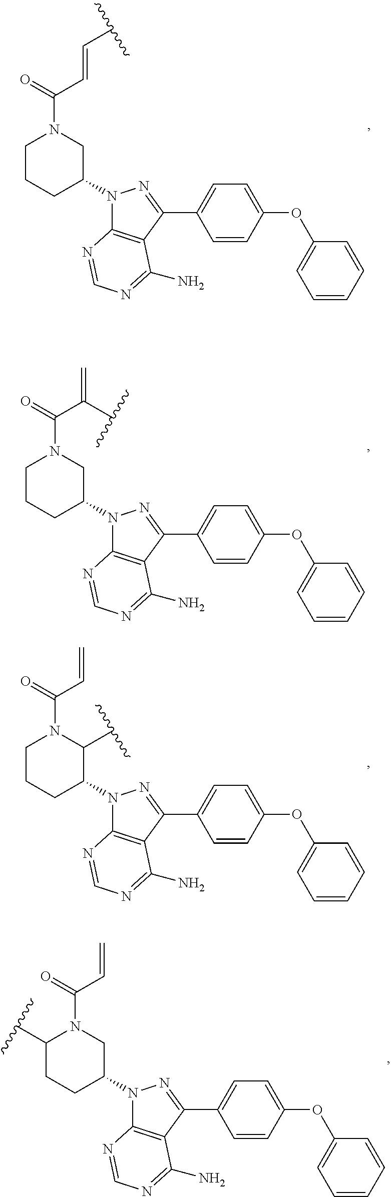

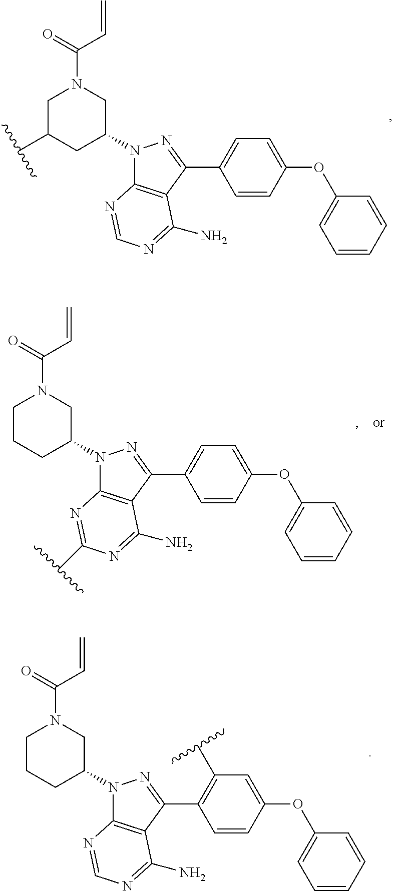 [SMILES: *C1C[C@@H](n2nc(-c3ccc(Oc4ccccc4)cc3)c3c(N)ncnc32)CN(C(=O)C=C)C1 ; *c1nc(N)c2c(-c3ccc(Oc4ccccc4)cc3)nn([C@@H]3CCCN(C(=O)C=C)C3)c2n1 ; *c1cc(Oc2ccccc2)ccc1-c1nn([C@@H]2CCCN(C(=O)C=C)C2)c2ncnc(N)c12]

In certain embodiments, the TPL is

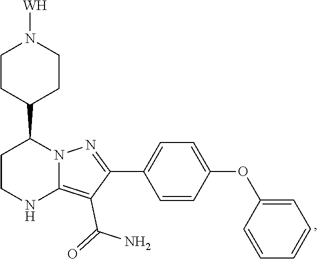

wherein WH is a group that reacts with BTK to form a covalent linkage.

In certain embodiments, the TPL is

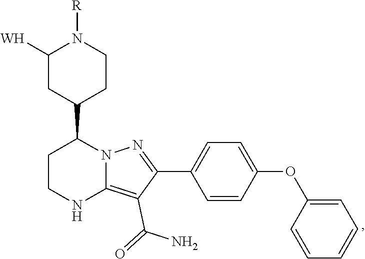

wherein WH is a group that reacts with BTK to form a covalent linkage, and R is H, alkyl, or acyl.

In certain embodiments, the TPL is one of the following:

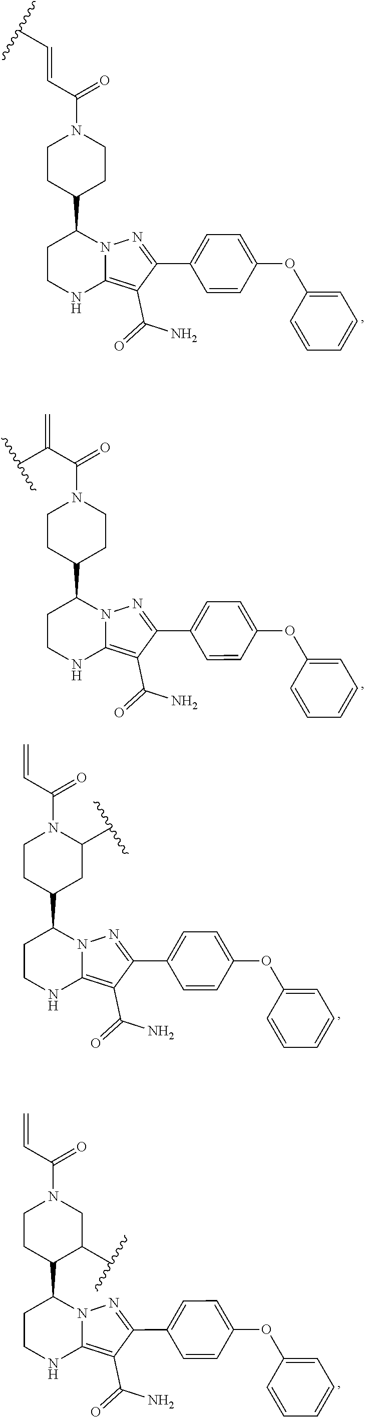 [SMILES: */C=C/C(=O)N1CCC([C@@H]2CCNc3c(C(N)=O)c(-c4ccc(Oc5ccccc5)cc4)nn32)CC1 ; *C(=C)C(=O)N1CCC([C@@H]2CCNc3c(C(N)=O)c(-c4ccc(Oc5ccccc5)cc4)nn32)CC1 ; *C1CC([C@@H]2CCNc3c(C(N)=O)c(-c4ccc(Oc5ccccc5)cc4)nn32)CCN1C(=O)C=C ; *C1CN(C(=O)C=C)CCC1[C@@H]1CCNc2c(C(N)=O)c(-c3ccc(Oc4ccccc4)cc3)nn21]

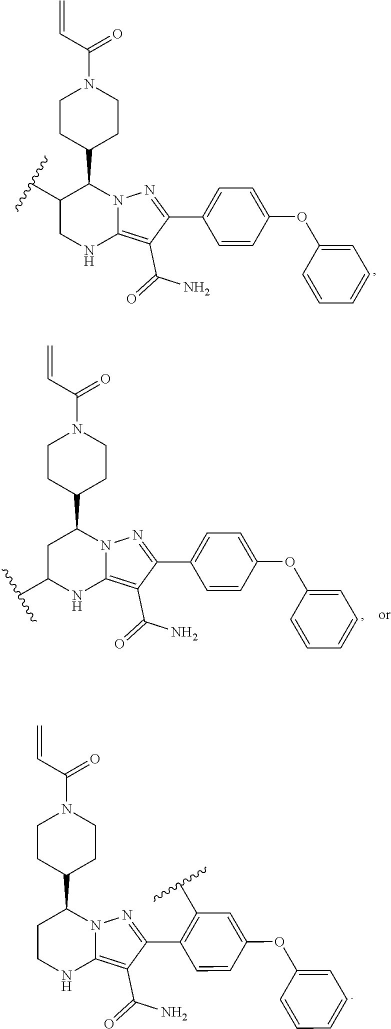 [SMILES: *C1CNc2c(C(N)=O)c(-c3ccc(Oc4ccccc4)cc3)nn2[C@@H]1C1CCN(C(=O)C=C)CC1 ; *C1C[C@@H](C2CCN(C(=O)C=C)CC2)n2nc(-c3ccc(Oc4ccccc4)cc3)c(C(N)=O)c2N1 ; *c1cc(Oc2ccccc2)ccc1-c1nn2c(c1C(N)=O)NCC[C@H]2C1CCN(C(=O)C=C)CC1]

In certain embodiments, the TPL is

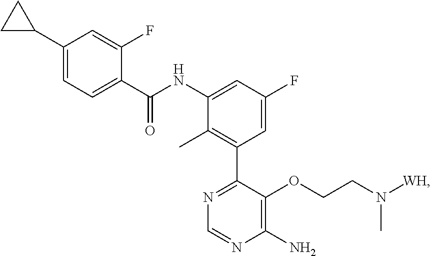

wherein WH is a group that reacts with BTK to form a covalent linkage.

In certain embodiments, the TPL is one of the following:

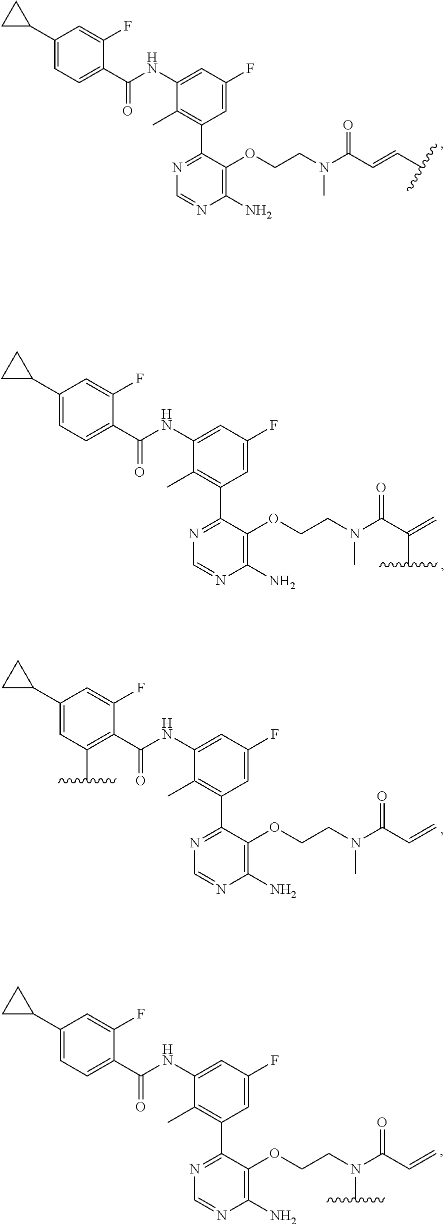 [SMILES: */C=C/C(=O)N(C)CCOc1c(N)ncnc1-c1cc(F)cc(NC(=O)c2ccc(C3CC3)cc2F)c1C ; *C(=C)C(=O)N(C)CCOc1c(N)ncnc1-c1cc(F)cc(NC(=O)c2ccc(C3CC3)cc2F)c1C ; *c1cc(C2CC2)cc(F)c1C(=O)Nc1cc(F)cc(-c2ncnc(N)c2OCCN(C)C(=O)C=C)c1C ; *N(CCOc1c(N)ncnc1-c1cc(F)cc(NC(=O)c2ccc(C3CC3)cc2F)c1C)C(=O)C=C]

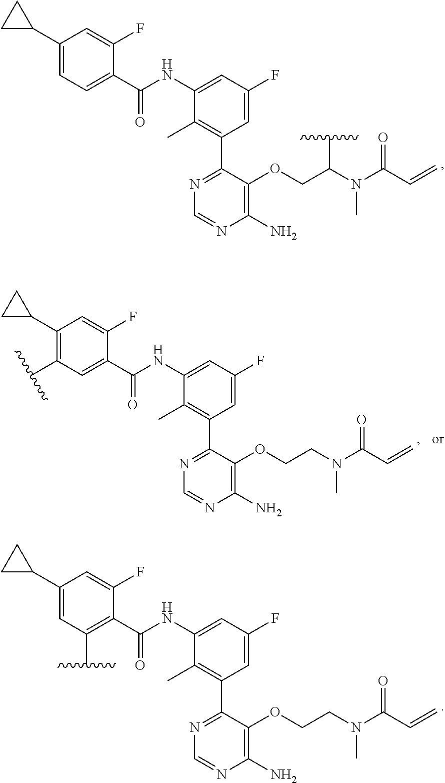 [SMILES: *C(COc1c(N)ncnc1-c1cc(F)cc(NC(=O)c2ccc(C3CC3)cc2F)c1C)N(C)C(=O)C=C ; *c1cc(C(=O)Nc2cc(F)cc(-c3ncnc(N)c3OCCN(C)C(=O)C=C)c2C)c(F)cc1C1CC1 ; *c1cc(C2CC2)cc(F)c1C(=O)Nc1cc(F)cc(-c2ncnc(N)c2OCCN(C)C(=O)C=C)c1C]

In certain embodiments, the TPL is a moiety that inhibits BTK. Compounds that inhibit BTK are reported in the literature, which include:

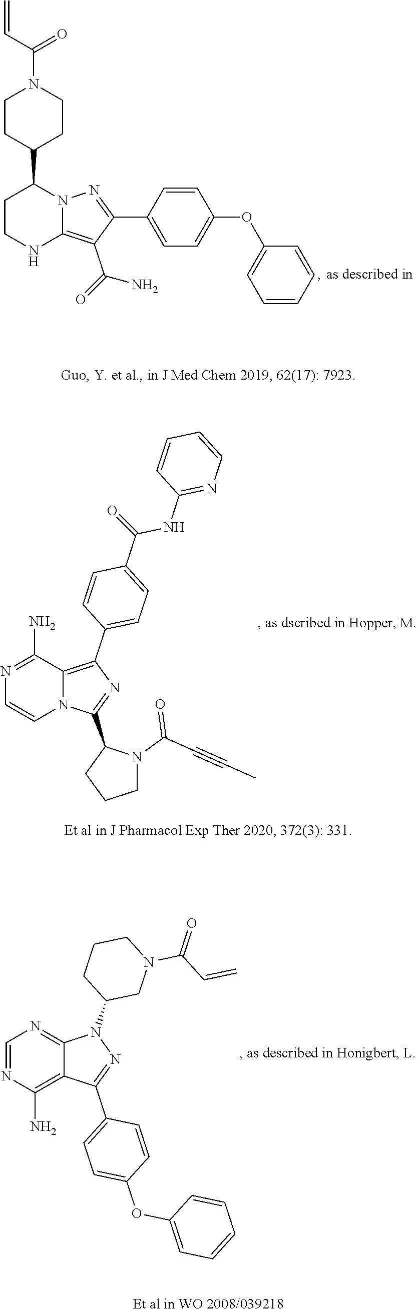 [SMILES: C=CC(=O)N1CCC([C@@H]2CCNc3c(C(N)=O)c(-c4ccc(Oc5ccccc5)cc4)nn32)CC1 ; CC#CC(=O)N1CCC[C@H]1c1nc(-c2ccc(C(=O)Nc3ccccn3)cc2)c2c(N)nccn12 ; C=CC(=O)N1CCC[C@@H](n2nc(-c3ccc(Oc4ccccc4)cc3)c3c(N)ncnc32)C1]

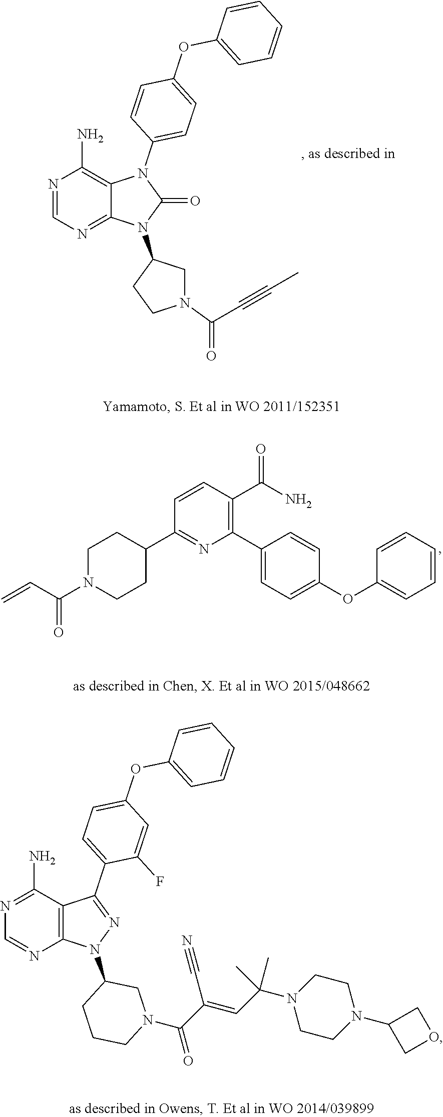 [SMILES: CC#CC(=O)N1CC[C@@H](n2c(=O)n(-c3ccc(Oc4ccccc4)cc3)c3c(N)ncnc32)C1 ; C=CC(=O)N1CCC(c2ccc(C(N)=O)c(-c3ccc(Oc4ccccc4)cc3)n2)CC1 ; CC(C)(/C=C(\C#N)C(=O)N1CCC[C@@H](n2nc(-c3ccc(Oc4ccccc4)cc3F)c3c(N)ncnc32)C1)N1CCN(C2COC2)CC1]

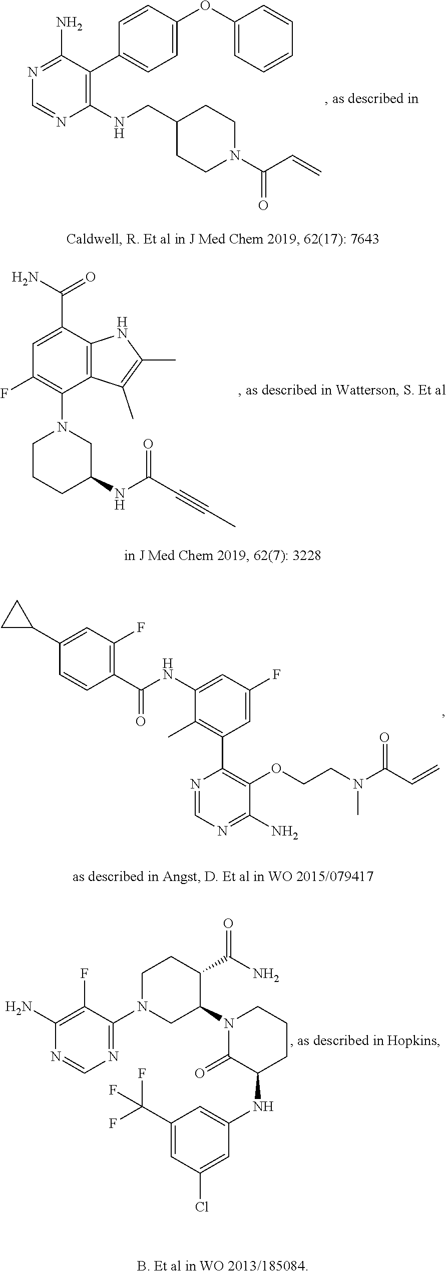 [SMILES: C=CC(=O)N1CCC(CNc2ncnc(N)c2-c2ccc(Oc3ccccc3)cc2)CC1 ; CC#CC(=O)N[C@H]1CCCN(c2c(F)cc(C(N)=O)c3[nH]c(C)c(C)c23)C1 ; C=CC(=O)N(C)CCOc1c(N)ncnc1-c1cc(F)cc(NC(=O)c2ccc(C3CC3)cc2F)c1C ; NC(=O)[C@H]1CCN(c2ncnc(N)c2F)C[C@@H]1N1CCC[C@@H](Nc2cc(Cl)cc(C(F)(F)F)c2)C1=O]

In certain embodiments, the TPL is a radical of one of the above compounds, which is attached to L through a modifiable oxygen, nitrogen, or carbon atom.

### Moiety for AR

In certain embodiments, the TPL is a moiety that binds to androgen receptor (AR) protein. Exemplary compounds that bind to AR are reported in the literature, such as TMBC and 5N-bicalutamide. A radical of such compounds reported in the literature that bind AR are amenable for use in the present invention.

In certain embodiments, the TPL is one of the following:

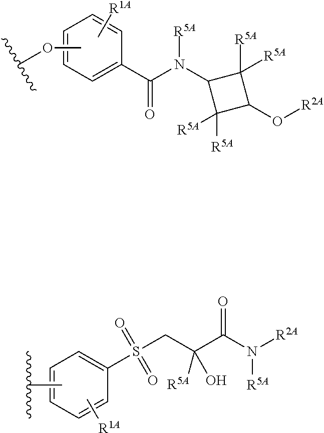

wherein:
R^{1A} is hydrogen, halo, hydroxyl, C₁₋₄ alkyl, C₁₋₄ alkoxyl, or —N(R^{5A})(R^{6A});
R^{2A} is -(phenyl or 5-6 membered heteroaryl containing 1, 2, or 3 heteroatoms independently selected from oxygen, nitrogen, and sulfur, wherein the phenyl and heteroaryl are optionally substituted with 1, 2, 3, or 5 substituents independently selected from halo, cyano, hydroxyl, C₁₋₄ alkyl, C₁₋₄ haloalkyl, and C₁₋₄ alkoxyl); and
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to same nitrogen atom are taken together with the nitrogen atom to which they are attached to form a 3-7 membered heterocyclic ring.

In certain embodiments, the TPL is one following:

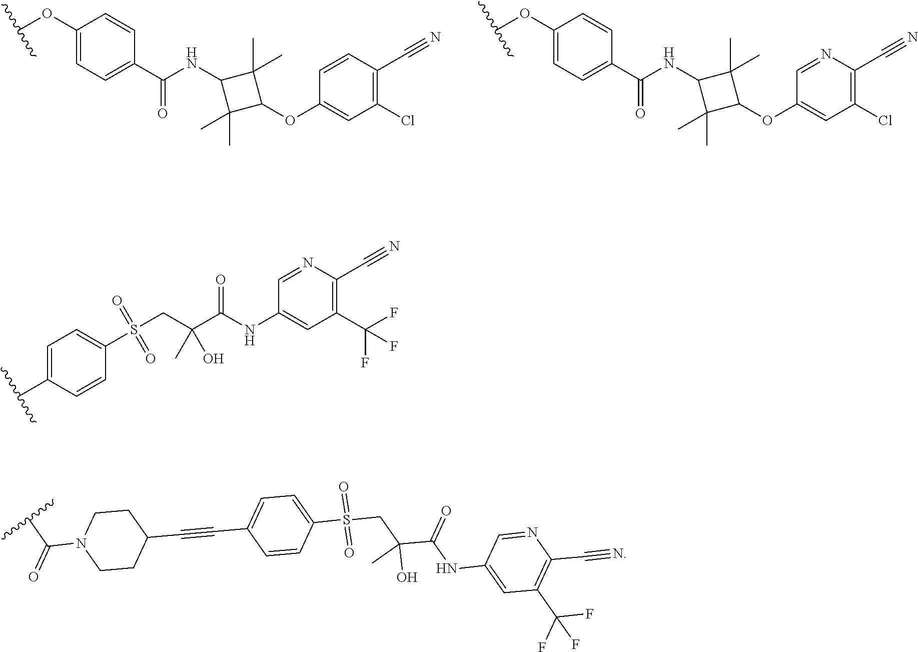 [SMILES: *Oc1ccc(C(=O)NC2C(C)(C)C(Oc3ccc(C#N)c(Cl)c3)C2(C)C)cc1 ; *Oc1ccc(C(=O)NC2C(C)(C)C(Oc3cnc(C#N)c(Cl)c3)C2(C)C)cc1 ; *c1ccc(S(=O)(=O)CC(C)(O)C(=O)Nc2cnc(C#N)c(C(F)(F)F)c2)cc1 ; *C(=O)N1CCC(C#Cc2ccc(S(=O)(=O)CC(C)(O)C(=O)Nc3cnc(C#N)c(C(F)(F)F)c3)cc2)CC1]

In certain embodiments, the TPL is

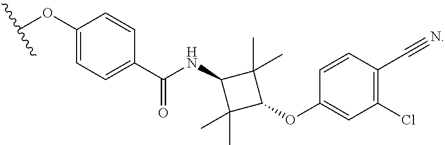 [SMILES: *Oc1ccc(C(=O)N[C@H]2C(C)(C)[C@H](Oc3ccc(C#N)c(Cl)c3)C2(C)C)cc1]

In certain embodiments, the TPL is

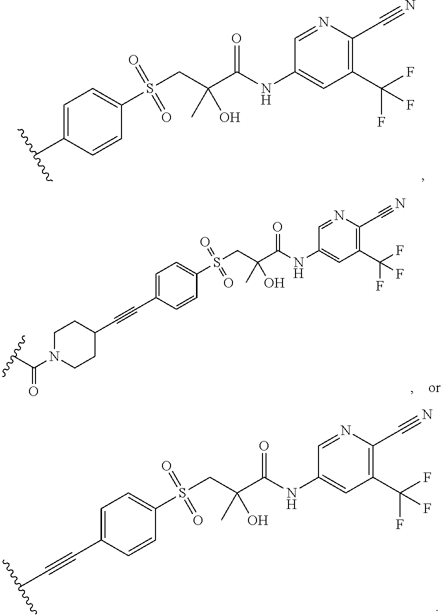 [SMILES: *c1ccc(S(=O)(=O)CC(C)(O)C(=O)Nc2cnc(C#N)c(C(F)(F)F)c2)cc1 ; *C(=O)N1CCC(C#Cc2ccc(S(=O)(=O)CC(C)(O)C(=O)Nc3cnc(C#N)c(C(F)(F)F)c3)cc2)CC1 ; *C#Cc1ccc(S(=O)(=O)CC(C)(O)C(=O)Nc2cnc(C#N)c(C(F)(F)F)c2)cc1]

In certain embodiments, the TPL is

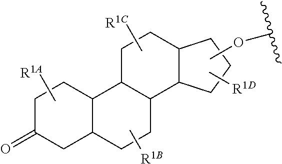

wherein R^{1A}, R^{1B}. R^{1C}, and R^{1D} are independently hydrogen, halo, hydroxyl, or C₁₋₄ alkyl.

In certain embodiments, the TPL is

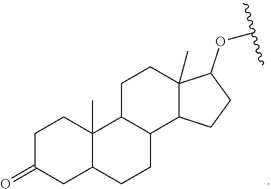 [SMILES: *OC1CCC2C3CCC4CC(=O)CCC4(C)C3CCC12C]

In certain embodiments, TPL is a moiety that is an agonist of the androgen receptor (AR) protein. Compounds that are agonists of the AR are reported in the literature, which include:

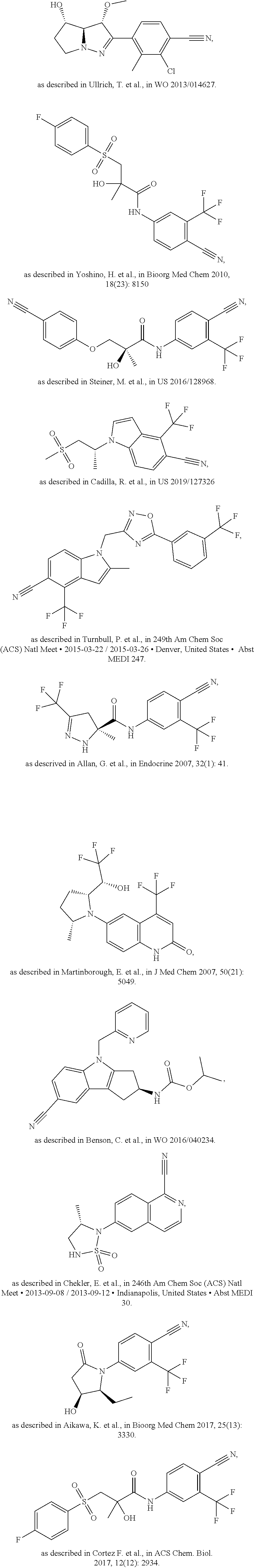

In certain embodiments, the TPL is a radical of one of the above compounds, which is attached to L through a modifiable oxygen, nitrogen, or carbon atom.

In certain embodiments, the TPL is a moiety that is an antagonist of the androgen receptor (AR). Compounds that are an antagonist of the AR are reported in the literature, which include:

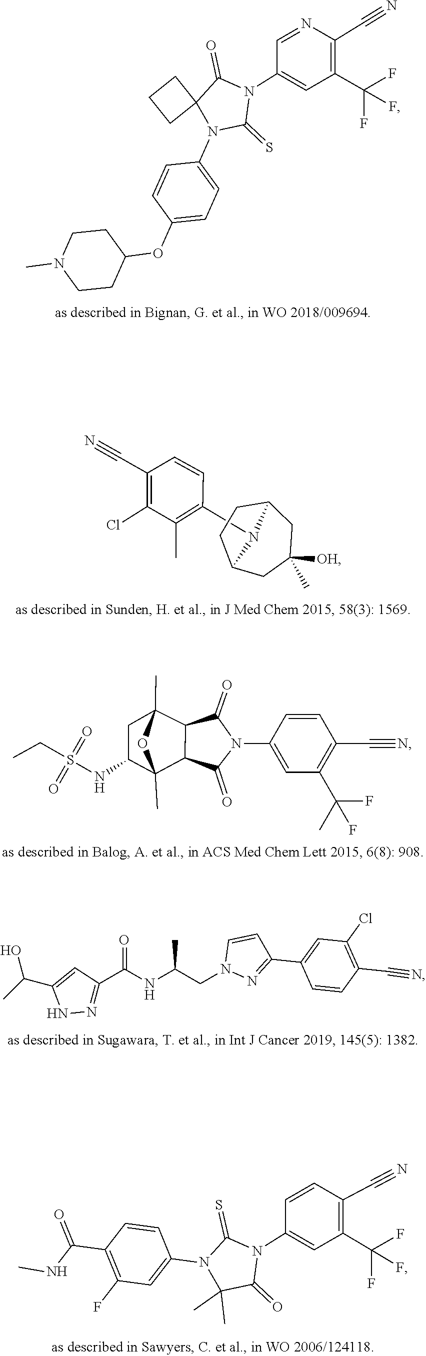 [SMILES: CN1CCC(Oc2ccc(N3C(=S)N(c4cnc(C#N)c(C(F)(F)F)c4)C(=O)C34CCC4)cc2)CC1 ; Cc1c(N2[C@@H]3CC[C@H]2C[C@@](C)(O)C3)ccc(C#N)c1Cl ; CCS(=O)(=O)N[C@@H]1C[C@@]2(C)O[C@]1(C)[C@@H]1C(=O)N(c3ccc(C#N)c(C(C)(F)F)c3)C(=O)[C@@H]12 ; CC(O)c1cc(C(=O)N[C@@H](C)Cn2ccc(-c3ccc(C#N)c(Cl)c3)n2)n[nH]1 ; CNC(=O)c1ccc(N2C(=S)N(c3ccc(C#N)c(C(F)(F)F)c3)C(=O)C2(C)C)cc1F]

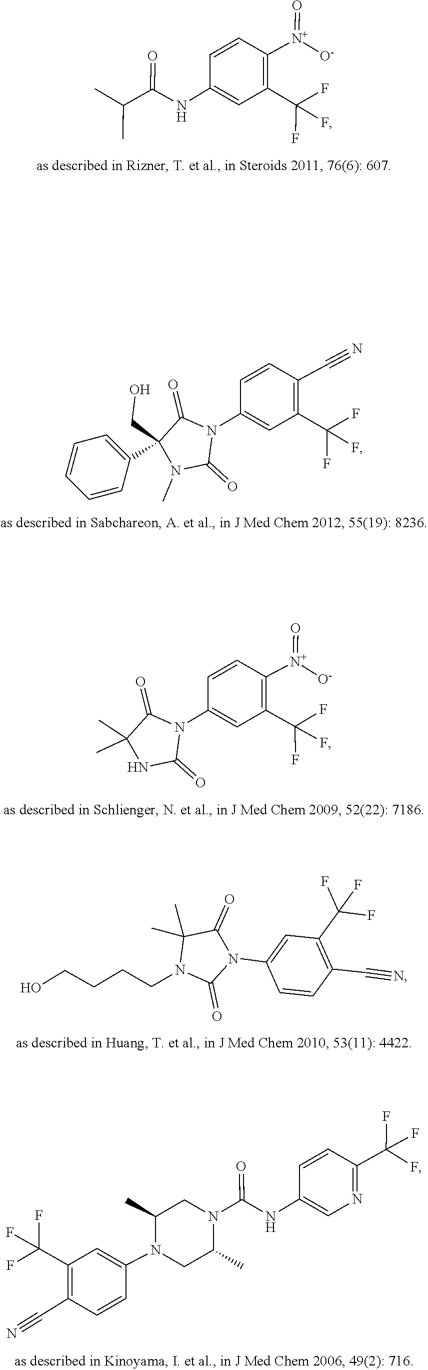 [SMILES: CC(C)C(=O)Nc1ccc([N+](=O)[O-])c(C(F)(F)F)c1 ; CN1C(=O)N(c2ccc(C#N)c(C(F)(F)F)c2)C(=O)[C@@]1(CO)c1ccccc1 ; CC1(C)NC(=O)N(c2ccc([N+](=O)[O-])c(C(F)(F)F)c2)C1=O ; CC1(C)C(=O)N(c2ccc(C#N)c(C(F)(F)F)c2)C(=O)N1CCCCO ; C[C@@H]1CN(c2ccc(C#N)c(C(F)(F)F)c2)[C@@H](C)CN1C(=O)Nc1ccc(C(F)(F)F)nc1]

In certain embodiments, the TPL is a radical of one of the above compounds, which is attached to L through a modifiable oxygen, nitrogen, or carbon atom.

### Moiety for IDH1

In certain embodiments, the TPL is a moiety that binds to and/or inhibits Isocitrate dehydrogenase 1 (IDH1). Compounds that bind and/or inhibit IDH1 are reported in the literature, which include ivosidenib (AG-120), AG-120 (racemic), vorasidenib (AG-881), BAY 1436032, and LY3410738. A radical of such compounds reported in the literature that bind IDH1 are amenable for use in the present invention. Additional exemplary compounds that inhibit and/or bind IDH1 are:

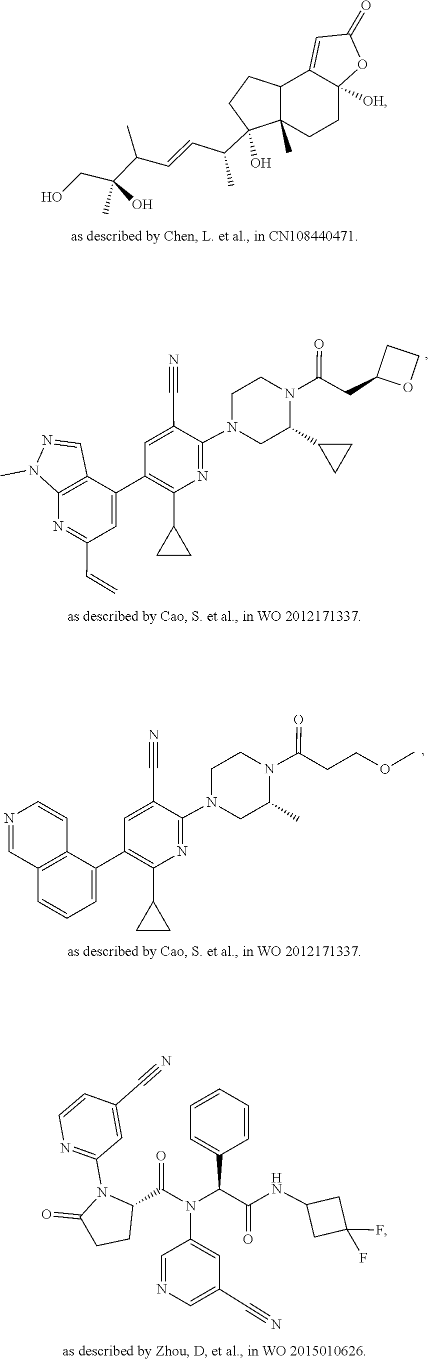

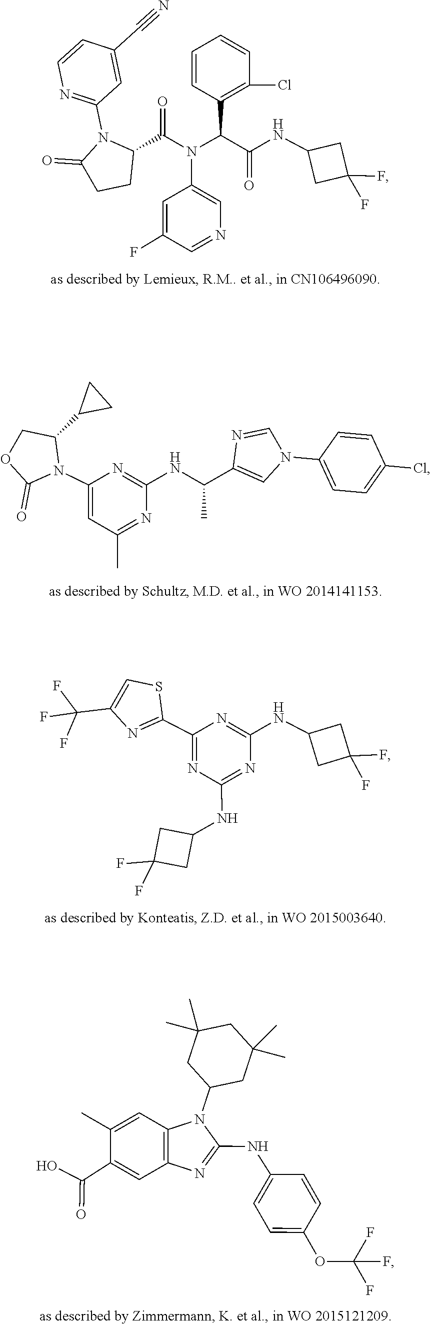

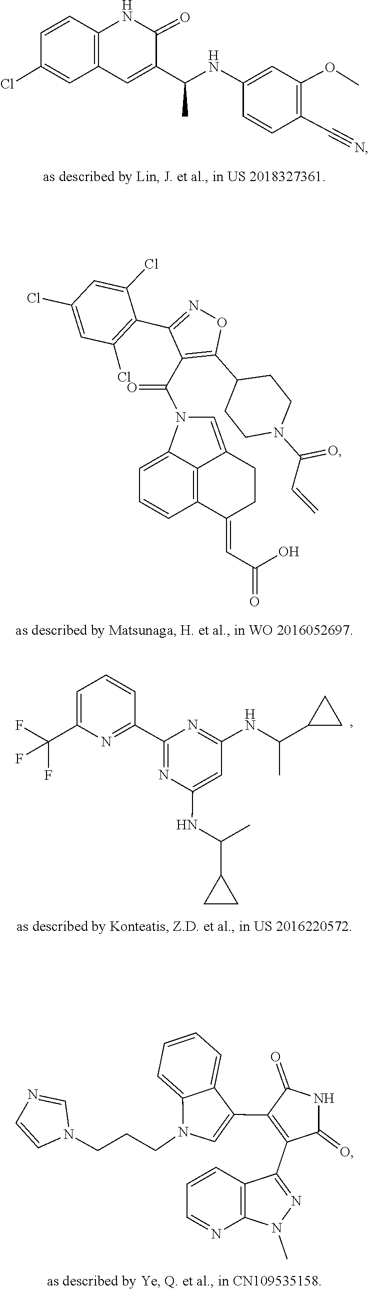

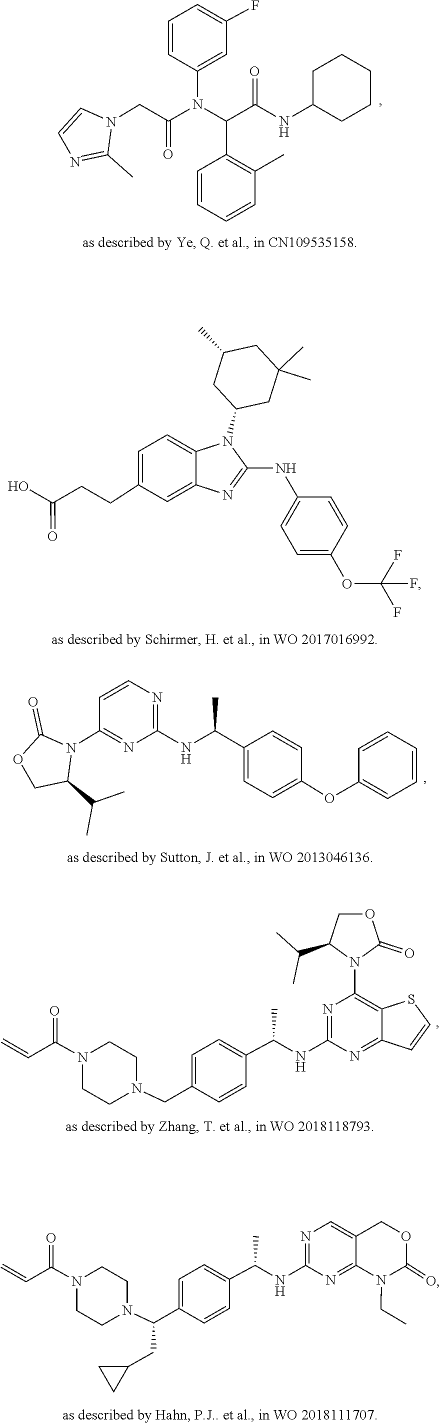

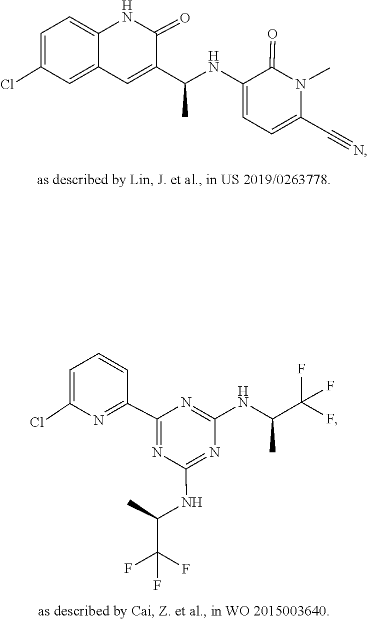

In certain embodiments, the TPL is a radical of one of the above compounds, which is attached to L through a modifiable oxygen, nitrogen, or carbon atom.

In certain embodiments, the TPL is

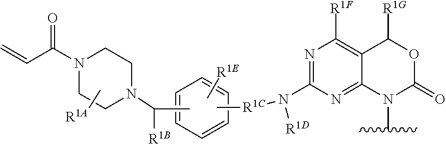

wherein:
R^{1A} and R^{1D} are independently hydrogen or C₁₋₄ alkyl;
R^{1B} is hydrogen, C₁₋₄alkyl, or (C₁₋₄ alkylene)-(C₃₋₆ cycloalkyl);
R^{1C} is C₁₋₄alkylene;
R^{1E}, R^{1F}, and R^{1G} are independently hydrogen, halo, hydroxyl, C₁₋₄ alkyl, or C₁₋₄ alkoxyl.

In certain embodiments, the TPL is

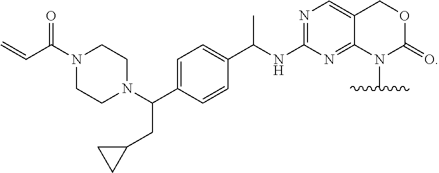 [SMILES: *N1C(=O)OCc2cnc(NC(C)c3ccc(C(CC4CC4)N4CCN(C(=O)C=C)CC4)cc3)nc21]

In certain embodiments, the TPL is

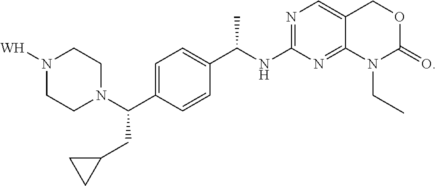

wherein WH is a group that reacts with IDH1 to form a covalent linkage. In certain embodiments, the TPL is one of the following:

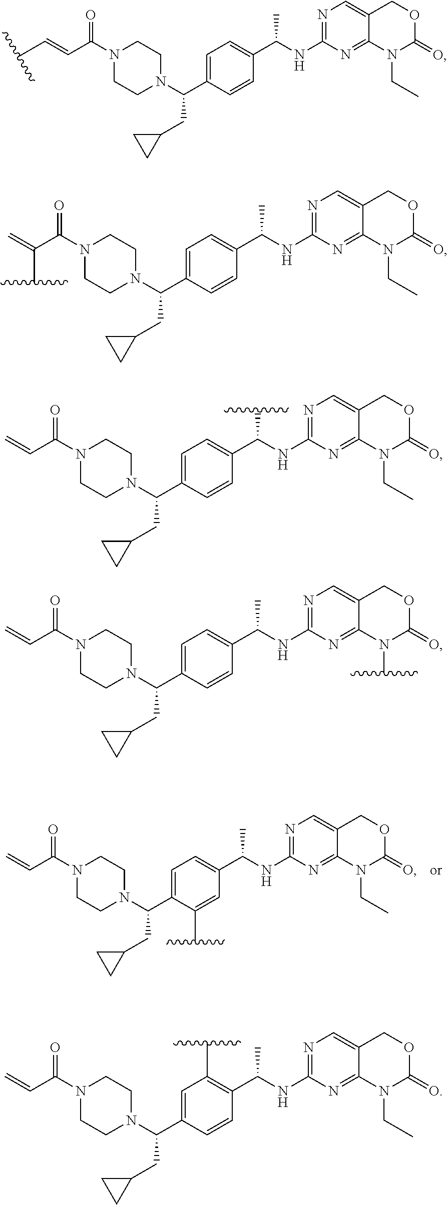

In certain embodiments, the TPL is selected from those depicted in the compounds in Table 3, below. In certain embodiments, the TPL is selected from those depicted in the compounds in Table 1 or 1-A, below.

### Additional Features

Certain embodiments above describe compounds and/or moieties that contain a warhead (WH) group. In certain embodiments, the WH group is R^{WH}, which is an electrophilic group capable of reacting with a protein, such as reacting with a nucleophilic functional group of a protein, such as a sulfhydryl group of a cysteine residue or an amino group of a lysine residue.

In certain embodiments, R^{WH} is -L^{W}-Y^{W}, wherein:
L^{W} is a covalent bond or a bivalent C₁₋₈ saturated or unsaturated, straight or branched, hydrocarbon chain, wherein one, two, or three methylene units of L^{W} are optionally and independently replaced by cyclopropylene, —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, —C(═S)—, —C(═NH)—, —N═N—, or —C(═N₂); and
Y^{W} is —C(O)—(C₂₋₆ alkenyl), —C(O)—(C₂₋₆ fluoroalkenyl), —C(O)—(C₂₋₆ alkynyl), —S(O)₂—(C₂₋₆ alkenyl), —S(O)₂—(C₂₋₆ fluoroalkenyl), —S(O)₂—(C₂₋₆ alkynyl), —S(O)₂—F, C₁₋₆ chloroalkyl, C₁₋₆ bromoalkyl, —(C₂₋₆ nitroalkenyl), or chloroacetyl, each of which is optionally substituted.

In certain embodiments, R^{WH} is -L^{W}-Y^{W}, wherein:
L^{W} is a covalent bond or a bivalent C₁₋₈ saturated or unsaturated, straight or branched, hydrocarbon chain, wherein one, two, or three methylene units of L^{W} are optionally and independently replaced by cyclopropylene, —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, —C(═S)—, —C(═NH)—, —N═N—, or —C(═N₂); and

Y^{W} is

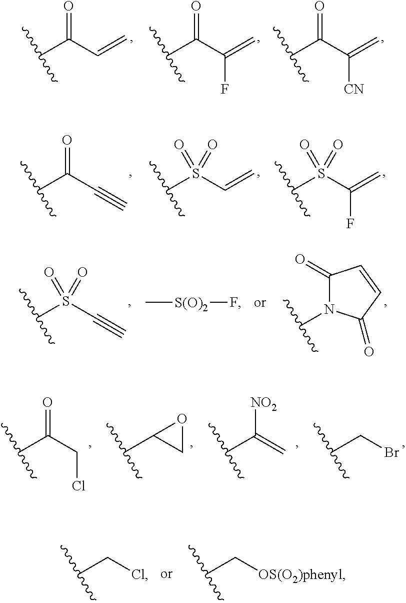

each of which is optionally substituted.

In certain embodiments, R^{WH} is —C(O)—(C₂₋₆ alkenyl), —C(O)—(C₂₋₆ fluoroalkenyl), —C(O)—(C₂₋₆ alkynyl), —S(O)₂—(C₂₋₆ alkenyl), —S(O)₂—(C₂₋₆ fluoroalkenyl), —S(O)₂—(C₂₋₆ alkynyl), —S(O)₂—F, C₁₋₆ chloroalkyl, C₁₋₆ bromoalkyl, —(C₂₋₆ nitroalkenyl), or chloroacetyl, each of which is optionally substituted. In certain embodiments, R^{WH} is

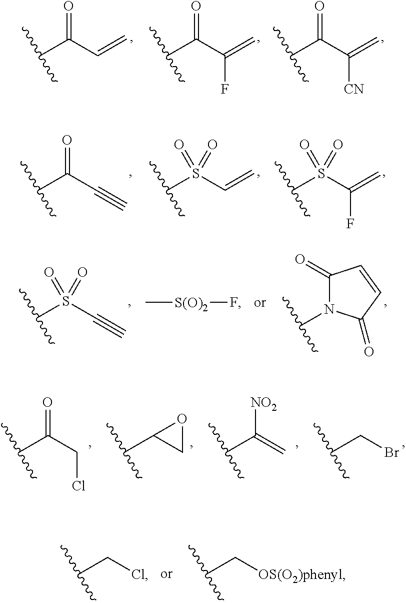

each of which is optionally substituted. In certain embodiments, R^{W} is

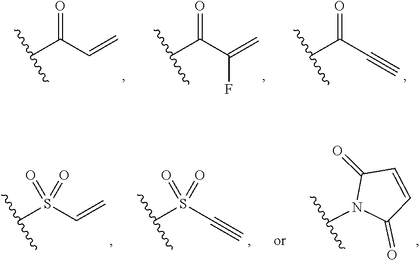

each of which is optionally substituted. In certain embodiments, R^{WH} is

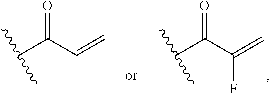

each of which is optionally substituted. In certain embodiments, R^{WH} is

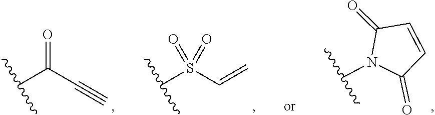

each of which is optionally substituted.

In certain embodiments, R^{WH} is —C(O)—(C₂₋₆ alkenyl), —C(O)—(C₂₋₆ fluoroalkenyl), —C(O)—(C₂₋₆ alkynyl), —S(O)₂—(C₂₋₆ alkenyl), —S(O)₂—(C₂₋₆ fluoroalkenyl), —S(O)₂—(C₂₋₆ alkynyl), —S(O)₂—F, C₁₋₆ chloroalkyl, C₁₋₆ bromoalkyl, —(C₂₋₆ nitroalkenyl), or chloroacetyl. In certain embodiments, R^{WH} is

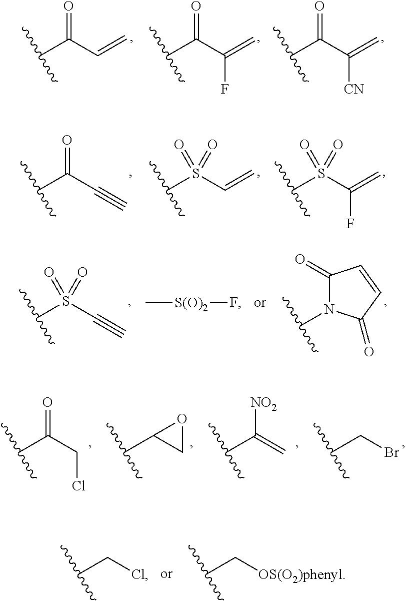

In certain embodiments, R^{WH} is

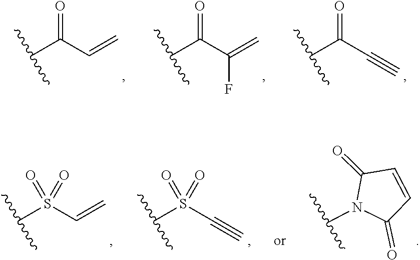

In certain embodiments, R^{WH} is

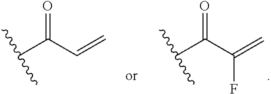

In certain embodiments, R^{W} is

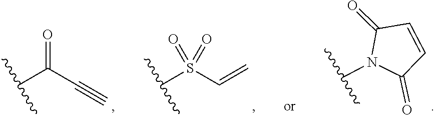

Compounds of Formula I may be further characterized according to the molecular weight of the TPL. In certain embodiments, the TPL has a molecular weight of less than 1500 Da, 1200 Da, 1000 Da, 800 Da, 600 Da, 400 Da, 300 Da, 200 Da, 150 Da, or 100 Da. Compounds of Formula II may be further characterized according to the molecular weight of the EPL. In certain embodiments, the EPL has a molecular weight of less than 1500 Da, 1200 Da, 1000 Da, 800 Da, 600 Da, 400 Da, 300 Da, 200 Da, 150 Da, or 100 Da.

### Part C: Exemplary Further Description of Linker (L) Component of Compounds of Formula I

Compounds of Formula I may be further characterized according to, for example, the identity of the linker (L) component. A variety of linkers are known to one of skill in the art and may be used in the heterobifunctional compounds described herein. For example, in certain embodiments, L comprises one or more optionally substituted groups selected from amino acids, polyether chains, aliphatic groups, and any combinations thereof. In certain embodiments, L consists of one or more optionally substituted groups selected from amino acids, polyether chains, aliphatic groups, and any combinations thereof. In certain embodiments, L consists of one or more groups selected from amino acids, polyether chains, aliphatic groups, and any combinations thereof.

In some embodiments, L is symmetrical. In some embodiments, L is asymmetric. In certain embodiments, L is a bond.

In certain embodiments, L is a covalent bond or a bivalent C₁₋₃₀ saturated or unsaturated, straight or branched, hydrocarbon chain, wherein 1-15 methylene units of L are optionally and independently replaced by cyclopropylene, —N(H)—, —N(C₁₋₄ alkyl)-, —N(C₃₋₅ cycloalkyl)-, —O—, —C(O)—, —OC(O)—, —C(O)O—, —S—, —S(O)—, —S(O)₂—, —S(O)₂N(H)—, —S(O)₂N(C₁₋₄ alkyl)-, —S(O)₂N(C₃₋₅ cycloalkyl)-, —N(H)C(O)—, —N(C₁₋₄ alkyl)C(O)—, —N(C₃₋₅ cycloalkyl)C(O)—, —C(O)N(H)—, —C(O)N(C₁₋₄ alkyl)-, —C(O)N(C₃₋₅ cycloalkyl)-, phenylene, an 8-10 membered bicyclic arylene, a 4-7 membered saturated or partially unsaturated carbocyclylene, an 8-10 membered bicyclic saturated or partially unsaturated carbocyclylene, a 3-7 membered saturated or partially unsaturated heterocyclylene having 1-2 heteroatoms independently selected from nitrogen, oxygen, and sulfur, an 8-10 membered bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur.

In certain embodiments, L is a bivalent, saturated or unsaturated, straight or branched C₁₋₆₀ hydrocarbon chain, wherein 0-20 methylene units of the hydrocarbon are independently replaced with —O—, —S—, —N(R**)—, —OC(O)—, —C(O)O—, —S(O)—, —S(O)₂—, —N(R**)S(O)₂—, —S(O)₂N(R**)—, —N(R**)C(O)—, —C(O)N(R**)—, —OC(O)N(R**)—, —N(R**)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur, wherein R** represents independently for each occurrence hydrogen, C₁₋₆ alkyl, or C₃₋₆ cycloalkyl.

In certain embodiments, L is a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur.

In certain embodiments, L is a bivalent, saturated or unsaturated, straight or branched C₁₋₆₀ hydrocarbon chain, wherein (i)0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur, and (ii)0-1 methylene units of the hydrocarbon are independently replaced with —C(O)—(C₂₋₆ alkenylene)-, —C(O)—(C₂₋₆ fluoroalkenylene)-, —C(O)—(C₂₋₆ alkynylene)-, —S(O)₂—(C₂₋₆ alkenylene)-, —S(O)₂—(C₂₋₆ fluoroalkenylene)-, —S(O)₂—(C₂₋₆ alkynylene)-, or —(C₁₋₆ alkylene substituted with one R^{WH})—, wherein R^{WH} is

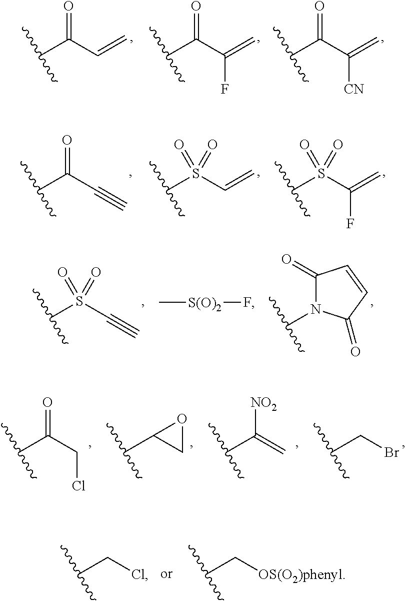

In certain embodiments, L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, —C(O)N(C₁₋₆ alkyl)-, 3-10 membered carbocyclyl, or 3-10 membered heterocyclyl containing 1, 2, or 3 heteroatoms independently selected from nitrogen, oxygen, and sulfur.

In certain embodiments, L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, or —C(O)N(C₁₋₆ alkyl)-.

In yet other embodiments, L comprises a polyethylene glycol chain ranging in size from about 1 to about 12 ethylene glycol units, from about 1 to about 10 ethylene glycol units, from about 2 to about 6 ethylene glycol units, from about 2 to about 5 ethylene glycol units, or from about 2 to about 4 ethylene glycol units. In yet other embodiments, L is a diradical of a polyethylene glycol chain ranging in size from about 1 to about 12 ethylene glycol units, from about 1 to about 10 ethylene glycol units, from about 2 to about 6 ethylene glycol units, from about 2 to about 5 ethylene glycol units, or from about 2 to about 4 ethylene glycol units.

In certain embodiments, L is a heteroalkylene having from 4 to 30 atoms selected from carbon, oxygen, nitrogen, and sulfur. In certain embodiments, L is a heteroalkylene having from 4 to 20 atoms selected from carbon, oxygen, nitrogen, and sulfur. In certain embodiments, L is a heteroalkylene having from 4 to 10 atoms selected from carbon, oxygen, nitrogen, and sulfur. In certain embodiments, L is a heteroalkylene having from 4 to 30 atoms selected from carbon, oxygen, and nitrogen. In certain embodiments, L is a heteroalkylene having from 4 to 20 atoms selected from carbon, oxygen, and nitrogen. In certain embodiments, L is a heteroalkylene having from 4 to 10 atoms selected from carbon, oxygen, and nitrogen. In certain embodiments, L is a heteroalkylene having from 4 to 30 atoms selected from carbon and oxygen. In certain embodiments, L is a heteroalkylene having from 4 to 20 atoms selected from carbon and oxygen. In certain embodiments, L is a heteroalkylene having from 4 to 10 atoms selected from carbon and oxygen.

In additional embodiments, the L is an optionally substituted (poly) ethyleneglycol having between 1 and about 100 ethylene glycol units, between about 1 and about 50 ethylene glycol units, between 1 and about 25 ethylene glycol units, between about 1 and about 10 ethylene glycol units, between 1 and about 8 ethylene glycol units, between 1 and about 6 ethylene glycol units, between 2 and about 4 ethylene glycol units, or optionally substituted alkyl groups interspersed with optionally substituted, O, N, S, P or Si atoms. In certain embodiments, L is substituted with an aryl, phenyl, benzyl, alkyl, alkylene, or heterocycle group.

In certain embodiments, L is a bivalent, saturated or unsaturated, straight or branched C₁₋₄₅ hydrocarbon chain, wherein 0-10 methylene units of the hydrocarbon are independently replaced with —O—, —S—, —N(R**)—, —OC(O)—, —C(O)O—, —S(O)—, —S(O)₂—, —N(R**)S(O)₂—, —S(O)₂N(R**)—, —N(R**)C(O)—, —C(O)N(R**)—, —OC(O)N(R**)—, —N(R**)C(O)O—, optionally substituted carbocyclyl, or optionally substituted heterocyclyl, wherein R** represents independently for each occurrence hydrogen, C₁₋₆ alkyl, or C₃₋₆ cycloalkyl.

In certain embodiments, L is a bivalent, saturated or unsaturated, straight or branched C₁₋₄₅ hydrocarbon chain, wherein 0-10 methylene units of the hydrocarbon are independently replaced with —O—, —S—, —N(R**)—, —OC(O)—, —C(O)O—, —S(O)—, —S(O)₂—, —N(R**)S(O)₂—, —S(O)₂N(R**)—, —N(R**)C(O)—, —C(O)N(R**)—, —OC(O)N(R**)—, —N(R**)C(O)O—, optionally substituted 3-10 membered carbocyclyl, or optionally substituted 3-10 membered heterocyclyl containing 1, 2, 3, or 4 heteroatoms selected from nitrogen, oxygen, and sulfur, wherein R** represents independently for each occurrence hydrogen, C₁₋₆ alkyl, or C₃₋₆ cycloalkyl.

In certain embodiments, L has the formula-N(R)-(optionally substituted 3-20 membered heteroalkylene)ₚ-CH₂—C(O)—, wherein R is hydrogen or optionally substituted C₁-C₆ alkyl, and p is 0 or 1.

In certain embodiments, L has the formula-N(R)-(3-20 membered heteroalkylene) p-CH₂—C(O)—; wherein the 3-20 membered heteroalkylene is optionally substituted with 1, 2, 3, or 4 substituents independently selected from halogen, C₁-C₆ haloalkyl, C₃-C₆ cycloalkyl, hydroxyl, and cyano; R is hydrogen or optionally substituted C₁-C₆ alkyl; and p is 0 or 1.

In certain embodiments, L has the formula-N(R)-(3-20 membered heteroalkylene) p-CH₂—C(O)—; wherein the 3-20 membered heteroalkylene is optionally substituted with 1, 2, or 3 substituents independently selected from halogen and C₁-C₆ haloalkyl; R is hydrogen or C₁-C₆ alkyl; and p is 0 or 1.

In I certain embodiments, L is —N(H)—(C₂₋₉ alkylene)-O—(C₁₋₆ alkylene)-C(O)—***, —N(H)—(C₁₀₋₂₀ alkylene)-O—(C₁₋₆ alkylene)-C(O)—***, —N(H)—[(C₂₋₄ alkylene)-O-]₂₋₆-(C₁₋₆ alkylene)-C(O)—***, —N(H)—[(C₂₋₄ alkylene)-O-]₇₋₁₅-(C₁₋₆ alkylene)-C(O)—***, —N(H)—(C₁₋₆ alkylene)-C(O)—***, —N(H)—(C₇₋₁₅ alkylene)-C(O)—***, —N(H)—[(C₂₋₄ alkylene)-O-]₂₋₆-(C₁₋₆ alkylene)-***, —N(H)—[(C₂₋₄ alkylene)-O-]₇₋₁₅-(C₁₋₆ alkylene)-***, —N(H)—(C₂₋₉ alkylene)-O—(C₁₋₆ alkylene)-C(O)N(C₁₋₆ alkyl)-(C₁₋₆ alkylene)-***, —N(H)—(C₂₋₉ alkylene)-O—(C₁₋₆ alkylene)-C(O)N(H)—(C₁₋₆ alkylene)-***, —N(H)—[(C₂₋₄ alkylene)-O-]₂₋₆-(C₁₋₆ alkylene)-N(H)—(C₁₋₆ alkylene)-***, —N(H)—[(C₂₋₄ alkylene)-O-]₇₋₁₅-(C₁₋₆ alkylene)-N(H)—(C₁₋₆ alkylene)-***, —N(H)—[(C₂₋₄ alkylene)-O-]₂₋₆-(C₁₋₆ alkylene)-N(C₁₋₆ alkyl)-(C₁₋₆ alkylene)-***, or —N(H)—[(C₂₋₄ alkylene)-O-]₇₋₁₅-(C₁₋₆ alkylene)-N(C₁₋₆ alkyl)-(C₁₋₆ alkylene)-***, where *** is a point of attachment to TPL.

In certain embodiments, L is —N(H)—(C₂₋₉ alkylene)-O—(C₁₋₆ alkylene)-C(O)—***, —N(H)—(C₁₀₋₂₀ alkylene)-O—(C₁₋₆ alkylene)-C(O)—***, —N(H)—[CH₂CH₂—O-]₂₋₆-(C₁₋₆ alkylene)-C(O)—***, —N(H)—[CH₂CH₂—O-]₇₋₁₅-(C₁₋₆ alkylene)-C(O)—***, —N(H)—(C₁₋₆ alkylene)-C(O)—***, —N(H)—(C₇₋₁₅ alkylene)-C(O)—***, —N(H)—[CH₂CH₂—O-]₂₋₆-(C₁₋₆ alkylene)-***, —N(H)—[CH₂CH₂—O-]₇₋₁₅-(C₁₋₆ alkylene)-***, —N(H)—(C₂₋₉ alkylene)-O—(C₁₋₆ alkylene)-C(O)N(C₁₋₆ alkyl)-(C₁₋₆ alkylene)-***, —N(H)—(C₂₋₉ alkylene)-O—(C₁₋₆ alkylene)-C(O)N(H)—(C₁₋₆ alkylene)-***, —N(H)—[CH₂CH₂—O-]₂₋₆-(C₁₋₆ alkylene)-N(H)—(C₁₋₆ alkylene)-***, —N(H)—[CH₂CH₂—O-]₇₋₁₅-(C₁₋₆ alkylene)-N(H)—(C₁₋₆ alkylene)-***, —N(H)—[CH₂CH₂—O-]₂₋₆-(C₁₋₆ alkylene)-N(C₁₋₆ alkyl)-(C₁₋₆ alkylene)-***, or —N(H)—[CH₂CH₂—O-]₇₋₁₅-(C₁₋₆ alkylene)-N(C₁₋₆ alkyl)-(C₁₋₆ alkylene)-***, where *** is a point of attachment to TPL.

In certain embodiments, L is —N(H)—[(C₂₋₄ alkylene)-O-]₂₋₆-(C₁₋₆ alkylene)-C(O)—***, —N(H)—[(C₂₋₄ alkylene)-O-]₇₋₁₅-(C₁₋₆ alkylene)-C(O)—***, —N(H)—(C₁₋₆ alkylene)-N(C₁₋₆ alkyl)C(O)—(C₁₋₆ alkylene) ***, —N(H)—(C₁₋₆ alkylene)-N(H)C(O)—(C₁₋₆ alkylene) ***, —N(H)—(C₂₋₆ alkylene)-***, —N(H)—(C₇₋₁₅ alkylene)-***, —N(C₁₋₆ alkyl)-(C₂₋₆ alkylene)-***, —N(C₁₋₆ alkyl)-(C₇₋₁₅ alkylene)-***, —N(H)—[(C₂₋₄ alkylene)-O-]₂₋₆-(C₁₋₆ alkylene)-***, —N(H)—[(C₂₋₄ alkylene)-O-]₇₋₁₅-(C₁₋₆ alkylene)-***, —N(H)—(C₁₋₆ alkylene)-(3-6 membered heterocycloalkylene)-(C₁₋₆ alkylene)-N(C₁₋₆ alkyl)-(C₁₋₆ alkylene)-***, —N(H)—(C₁₋₆ alkylene)-(3-6 membered heterocycloalkylene)-(C₁₋₆ alkylene)-N(H)—(C₁₋₆ alkylene)-***, —N(H)—(C₂₋₆ alkylene)-N(H)—(C₁₋₆ alkylene)-***, or —N(H)—(C₂₋₆ alkylene)-N(C₁₋₆ alkyl)-(C₁₋₆ alkylene)-***, where *** is a point of attachment to TPL.

In certain embodiments, L is —N(H)—[CH₂CH₂—O-]₂₋₆-(C₁₋₆ alkylene)-C(O)—***, —N(H)—[CH₂CH₂—O-]₇₋₁₅-(C₁₋₆ alkylene)-C(O)—***, —N(H)—(C₁₋₆ alkylene)-N(C₁₋₆ alkyl)C(O)—(C₁₋₆ alkylene) ***, —N(H)—(C₁₋₆ alkylene)-N(H)C(O)—(C₁₋₆ alkylene) ***, —N(H)—(C₂₋₆ alkylene)-***, —N(H)—(C₇₋₁₅ alkylene)-***, —N(C₁₋₆ alkyl)-(C₂₋₆ alkylene)-***, —N(C₁₋₆ alkyl)-(C₇₋₁₅ alkylene)-***, —N(H)—[CH₂CH₂—O-]₂₋₆-(C₁₋₆ alkylene)-***, —N(H)—[CH₂CH₂—O-]₇₋₁₅-(C₁₋₆ alkylene)-***, —N(H)—(C₁₋₆ alkylene)-(3-6 membered heterocycloalkylene)-(C₁₋₆ alkylene)-N(C₁₋₆ alkyl)-(C₁₋₆ alkylene)-***, —N(H)—(C₁₋₆ alkylene)-(3-6 membered heterocycloalkylene)-(C₁₋₆ alkylene)-N(H)—(C₁₋₆ alkylene)-***, —N(H)—(C₂₋₆ alkylene)-N(H)—(C₁₋₆ alkylene)-***, or —N(H)—(C₂₋₆ alkylene)-N(C₁₋₆ alkyl)-(C₁₋₆ alkylene)-***, where *** is a point of attachment to TPL.

In certain embodiments, L is —[(C₂₋₄ alkylene)-O-]₂₋₆-(C₁₋₆ alkylene)-***, —[(C₂₋₄ alkylene)-O-]₇₋₁₅-(C₁₋₆ alkylene)-***, —[(C₂₋₄ alkylene)-O-]₂₋₆-(C₁₋₆ alkylene)-N(C₁₋₆ alkyl)(C₁₋₆ alkylene)-***, —[(C₂₋₄ alkylene)-O-]₇₋₁₅-(C₁₋₆ alkylene)-N(C₁₋₆ alkyl)(C₁₋₆ alkylene)-***, —[(C₂₋₄ alkylene)-O-]₂₋₆-(C₁₋₆ alkylene)-N(H)(C₁₋₆ alkylene)-***, —[(C₂₋₄ alkylene)-O-]₇₋₁₅-(C₁₋₆ alkylene)-N(H)(C₁₋₆ alkylene)-***, —(C₁₋₉ alkylene)-C(O)N(H)—(C₁₋₆ alkylene)-***, —(C₁₋₉ alkylene)-N(H)C(O)—(C₁₋₆ alkylene)-***, —(C₁₋₉ alkylene)-C(O)N(H)—[(C₂₋₄ alkylene)-O-]₂₋₆-(C₁₋₆ alkylene)-***, —(C₁₋₉ alkylene)-N(H)C(O)—[(C₂₋₄ alkylene)-O-]₂₋₆-(C₁₋₆ alkylene)-***, —(C₁₋₉ alkylene)-C(O)N(H)—[(C₂₋₄ alkylene)-O-]₇₋₁₅-(C₁₋₆ alkylene)-***, —(C₁₋₉ alkylene)-N(H)C(O)—[(C₂₋₄ alkylene)-O-]₇₋₁₅-(C₁₋₆ alkylene)-***, —(C₁₋₉ alkylene)-C(O)N(H)—[(C₂₋₄ alkylene)-O-]₂₋₆-(C₁₋₆ alkylene)-N(C₁₋₆ alkyl)-(C₁₋₆ alkylene)-***, —(C₁₋₉ alkylene)-N(H)C(O)—[(C₂₋₄ alkylene)-O-]₂₋₆-(C₁₋₆ alkylene)-N(C₁₋₆ alkyl)-(C₁₋₆ alkylene)-***, —(C₁₋₉ alkylene)-C(O)N(H)—[(C₂₋₄ alkylene)-O-]₇₋₁₅-(C₁₋₆ alkylene)-N(C₁₋₆ alkyl)-(C₁₋₆ alkylene)-***, or —(C₁₋₉ alkylene)-N(H)C(O)—[(C₂₋₄ alkylene)-O-]₇₋₁₅-(C₁₋₆ alkylene)-N(C₁₋₆ alkyl)-(C₁₋₆ alkylene)-***, where *** is a point of attachment to TPL.

In certain embodiments, L is —[CH₂CH₂—O-]₂₋₆-(C₁₋₆ alkylene)-***, —[CH₂CH₂—O-]₇₋₁₅ —(C₁₋₆ alkylene)-***, —[CH₂CH₂—O-]₂₋₆-(C₁₋₆ alkylene)-N(C₁₋₆ alkyl)(C₁₋₆ alkylene)-***, —[CH₂CH₂—O-]₇₋₁₅-(C₁₋₆ alkylene)-N(C₁₋₆ alkyl)(C₁₋₆ alkylene)-***, —[CH₂CH₂—O-]₂₋₆-(C₁₋₆ alkylene)-N(H)(C₁₋₆ alkylene)-***, —[CH₂CH₂—O-]₇₋₁₅-(C₁₋₆ alkylene)-N(H)(C₁₋₆ alkylene)-***, —(C₁₋₉ alkylene)-C(O)N(H)—(C₁₋₆ alkylene)-***, —(C₁₋₉ alkylene)-N(H)C(O)—(C₁₋₆ alkylene)-***, —(C₁₋₉ alkylene)-C(O)N(H)—[CH₂CH₂—O-]₂₋₆-(C₁₋₆ alkylene)-***, —(C₁₋₉ alkylene)-N(H)C(O)—[CH₂CH₂—O-]₂₋₆-(C₁₋₆ alkylene)-***, —(C₁₋₉ alkylene)-C(O)N(H)—[CH₂CH₂—O-]₇₋₁₅-(C₁₋₆ alkylene)-***, —(C₁₋₉ alkylene)-N(H)C(O)—[CH₂CH₂—O-]₇₋₁₅-(C₁₋₆ alkylene)-***, —(C₁₋₉ alkylene)-C(O)N(H)—[CH₂CH₂—O-]₂₋₆-(C₁₋₆ alkylene)-N(C₁₋₆ alkyl)-(C₁₋₆ alkylene)-***, —(C₁₋₉ alkylene)-N(H)C(O)—[CH₂CH₂—O-]₂₋₆-(C₁₋₆ alkylene)-N(C₁₋₆ alkyl)-(C₁₋₆ alkylene)-***, —(C₁₋₉ alkylene)-C(O)N(H)—[CH₂CH₂—O-]₇₋₁₅-(C₁₋₆ alkylene)-N(C₁₋₆ alkyl)-(C₁₋₆ alkylene)-***, or —(C₁₋₉ alkylene)-N(H)C(O)—[(CH₂CH₂—O-]₇₋₁₅-(C₁₋₆ alkylene)-N(C₁₋₆ alkyl)-(C₁₋₆ alkylene)-***, where *** is a point of attachment to TPL.

In certain embodiments, L is —N(H)—[(C₂₋₄ alkylene)-O-]₂₋₆-(C₁₋₆ alkylene)-N(H)—***, —N(H)—[(C₂₋₄ alkylene)-O-]₇₋₁₅-(C₁₋₆ alkylene)-N(H)—***, —N(C₁₋₆ alkyl)-[(C₂₋₄ alkylene)-O-]₂₋₆-(C₁₋₆ alkylene)-N(H)—***, —N(C₁₋₆ alkyl)-[(C₂₋₄ alkylene)-O-]₇₋₁₅-(C₁₋₆ alkylene)-N(H)—***, —N(C₁₋₆ alkyl)-[(C₂₋₄ alkylene)-O-]₂₋₆-(C₁₋₆ alkylene)-N(C₁₋₆ alkyl)-***, or —N(C₁₋₆ alkyl)-[(C₂₋₄ alkylene)-O-]₇₋₁₅-(C₁₋₆ alkylene)-N(C₁₋₆ alkyl)-***, where *** is a point of attachment to TPL.

In certain embodiments, L is —N(H)—[CH₂CH₂—O-]₂₋₆-(C₁₋₆ alkylene)-N(H)—***, —N(H)—[CH₂CH₂—O-]₇₋₁₅-(C₁₋₆ alkylene)-N(H)—***, —N(C₁₋₆ alkyl)-[CH₂CH₂—O-]₂₋₆-(C₁₋₆ alkylene)-N(H)—***, —N(C₁₋₆ alkyl)-[CH₂CH₂—O-]₇₋₁₅-(C₁₋₆ alkylene)-N(H)—***, —N(C₁₋₆ alkyl)-[CH₂CH₂—O-]₂₋₆-(C₁₋₆ alkylene)-N(C₁₋₆ alkyl)-***, or —N(C₁₋₆ alkyl)-[CH₂CH₂—O-]₇₋₁₅-(C₁₋₆ alkylene)-N(C₁₋₆ alkyl)-***, where *** is a point of attachment to TPL.

In some embodiments. L is one of the following:

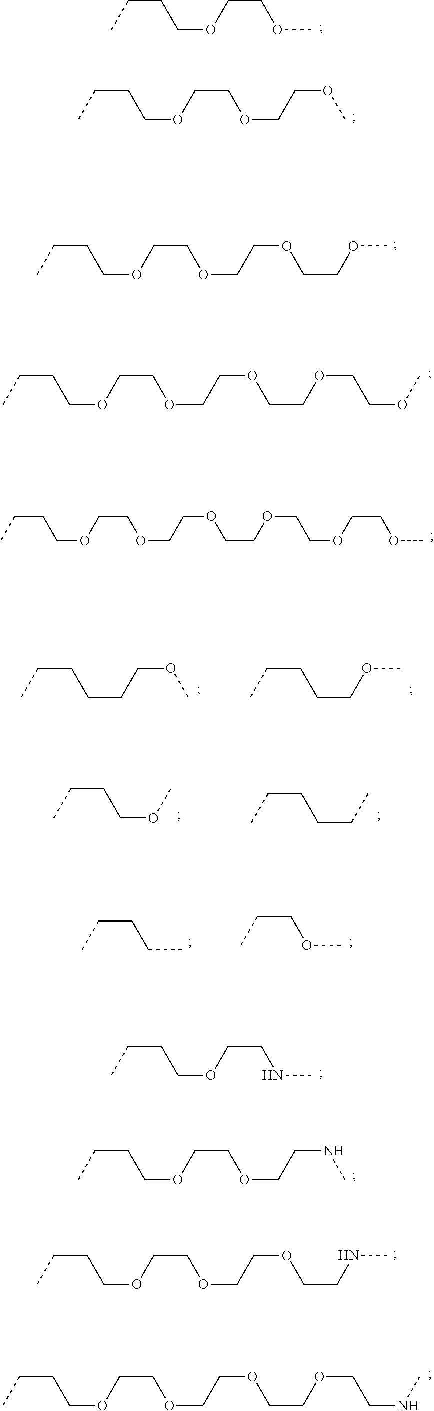

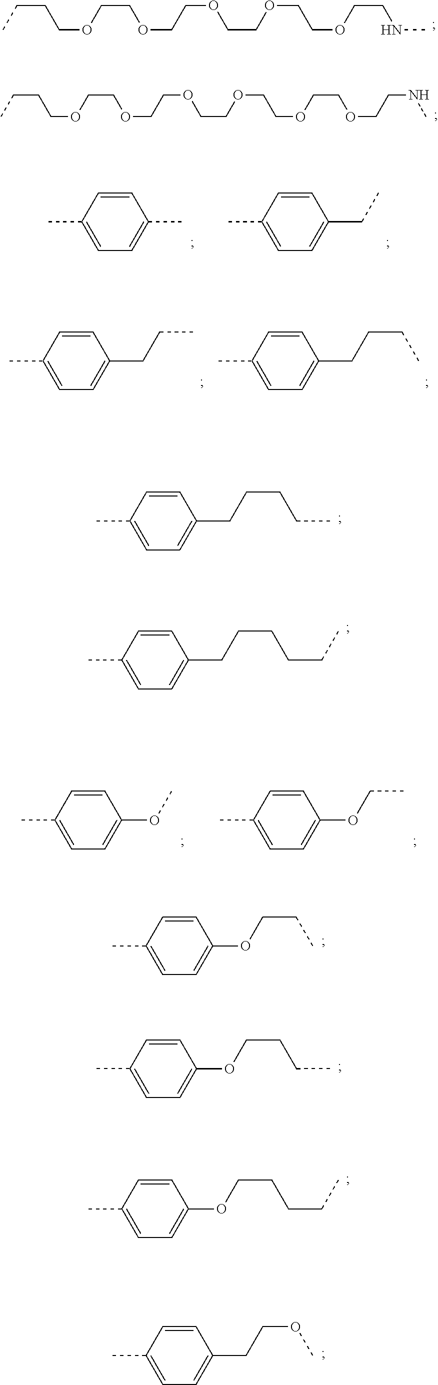

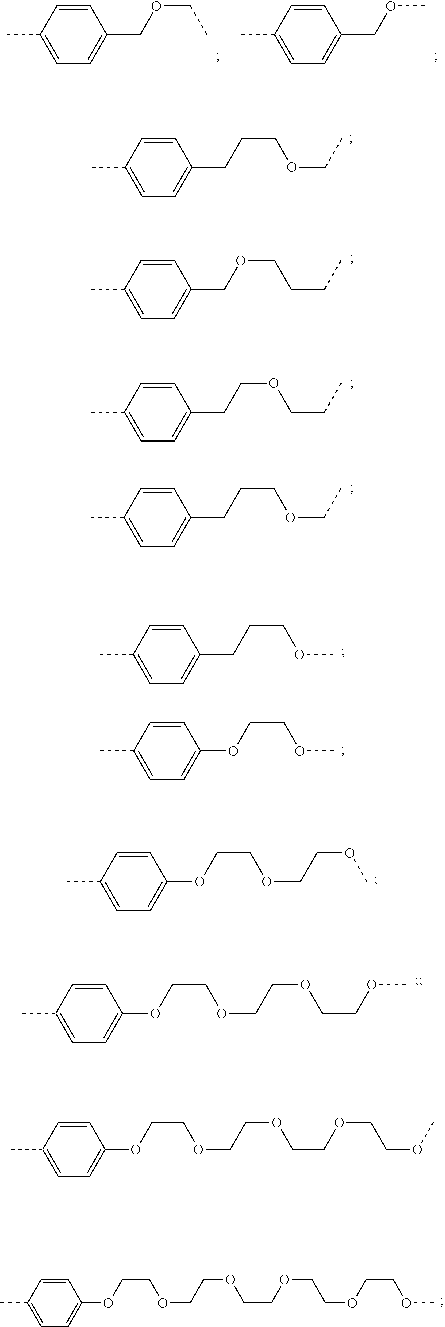

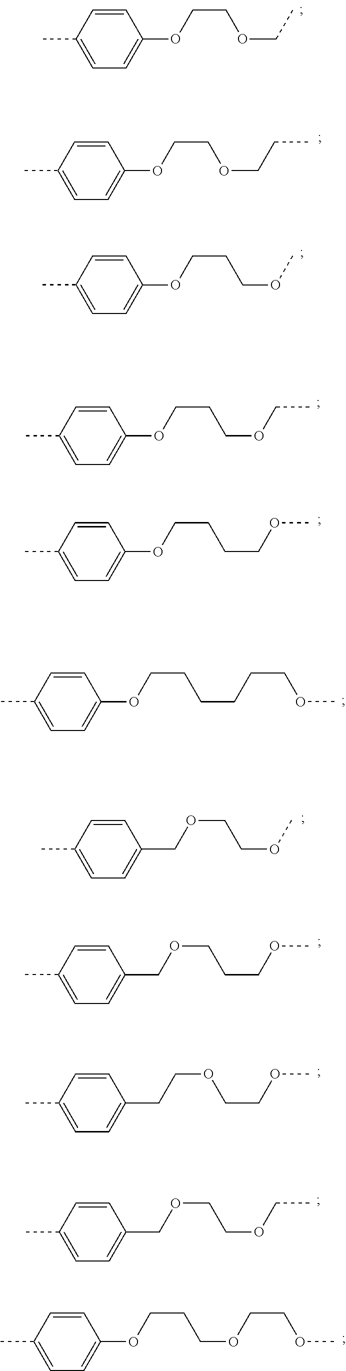

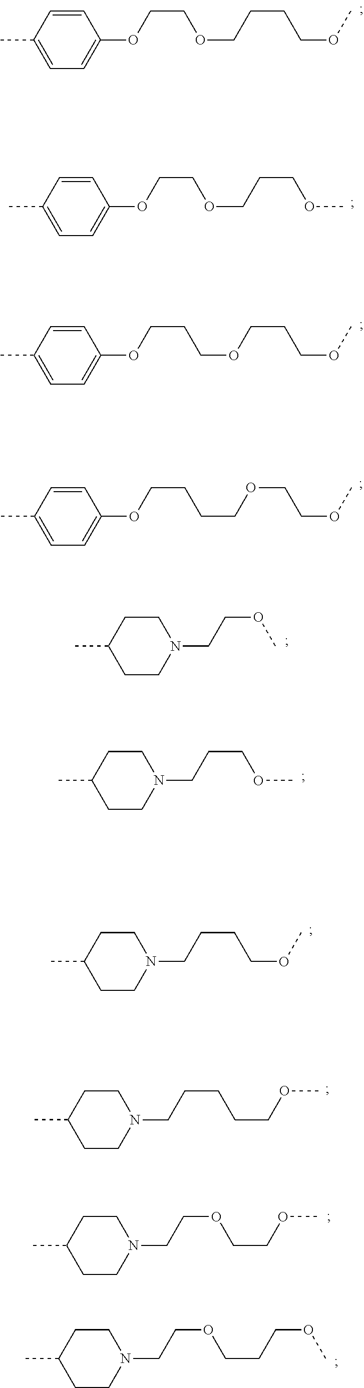

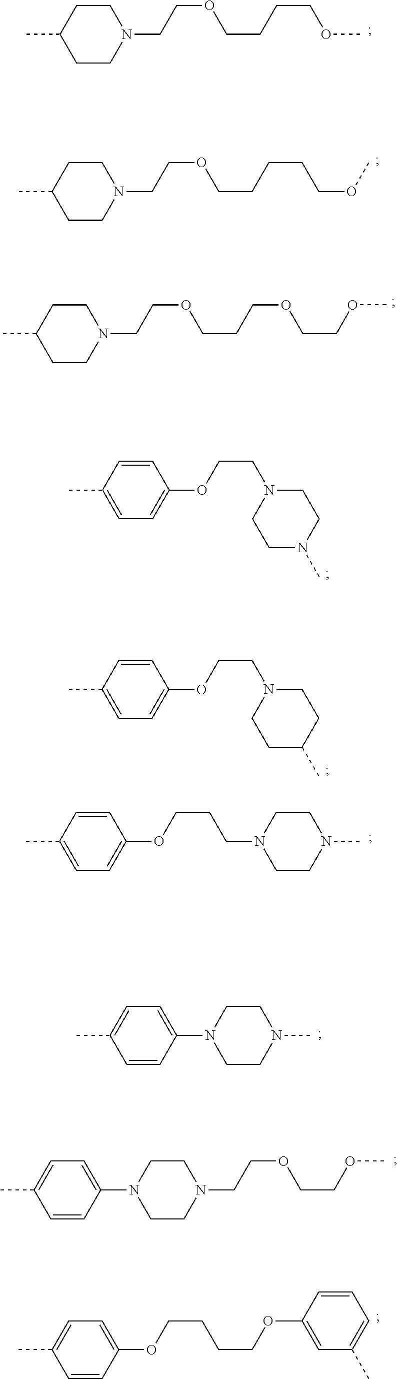

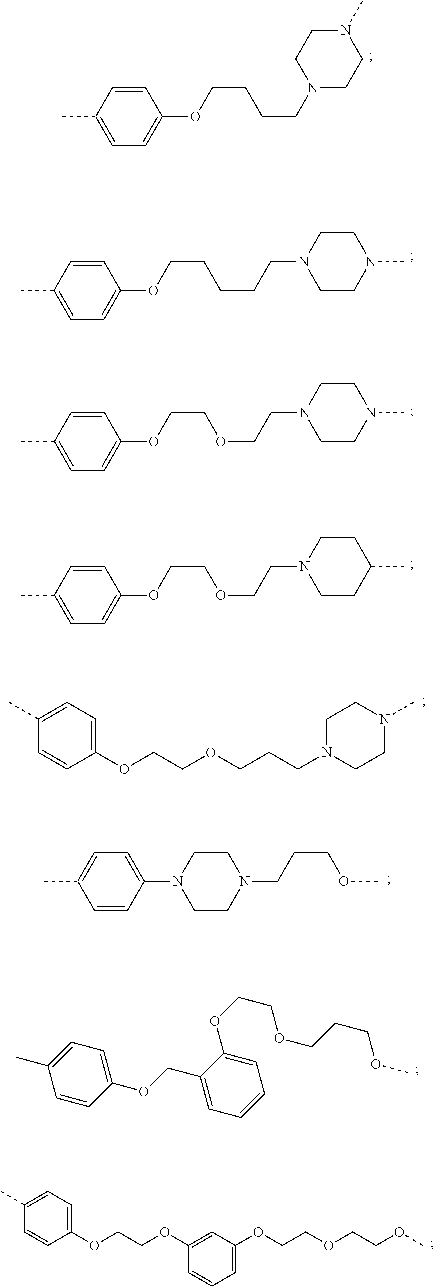

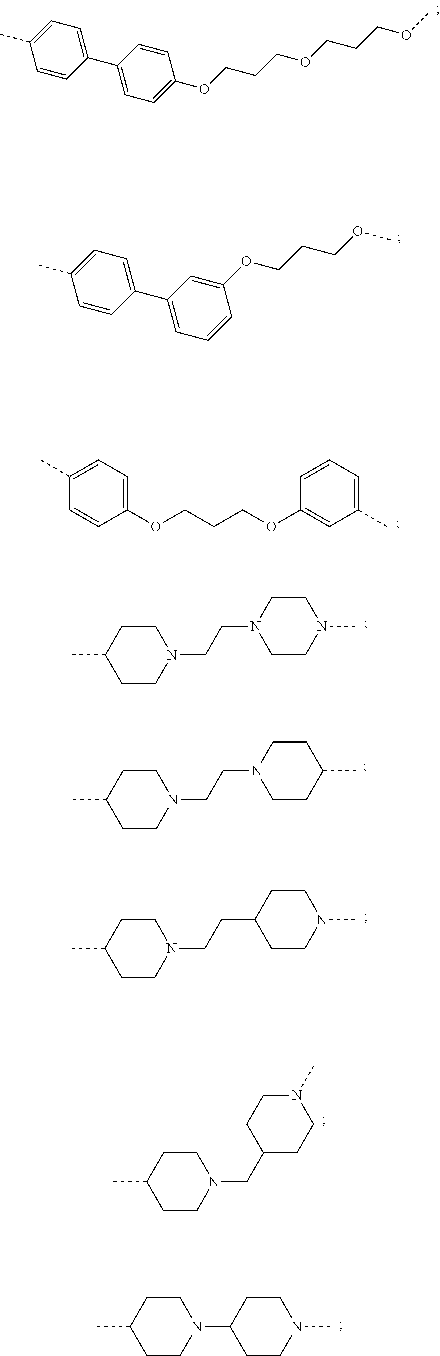

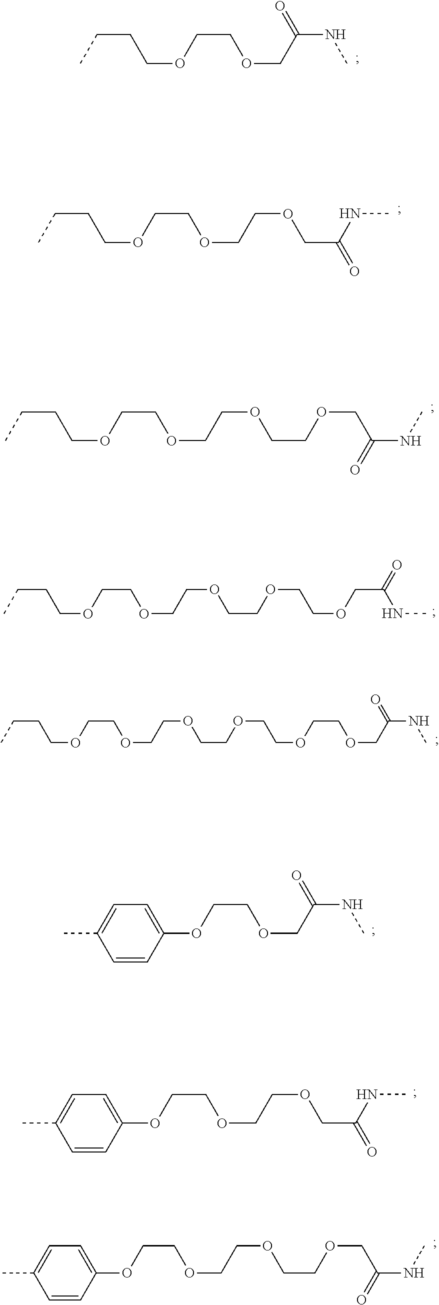

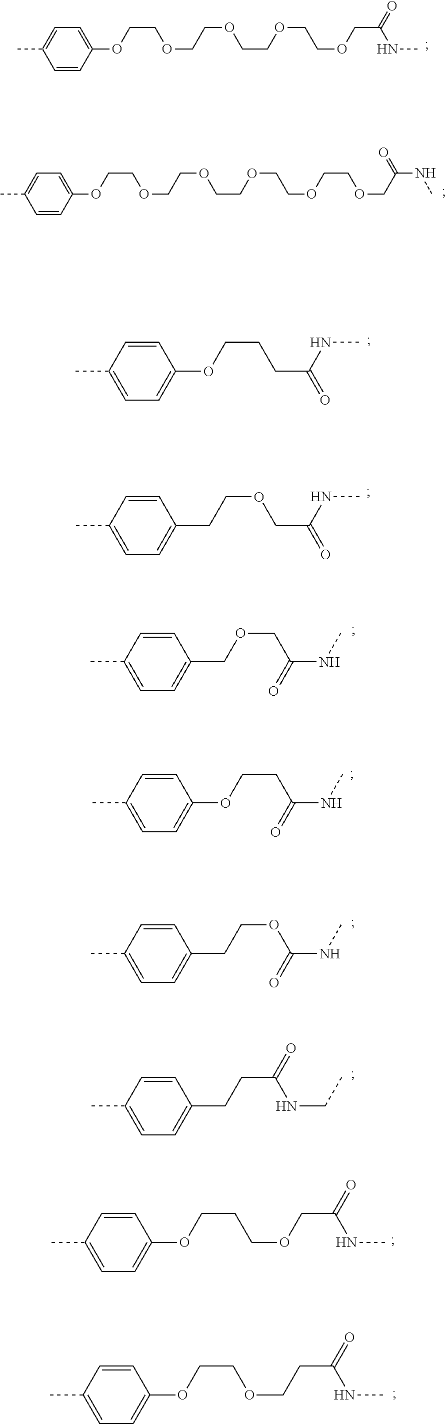

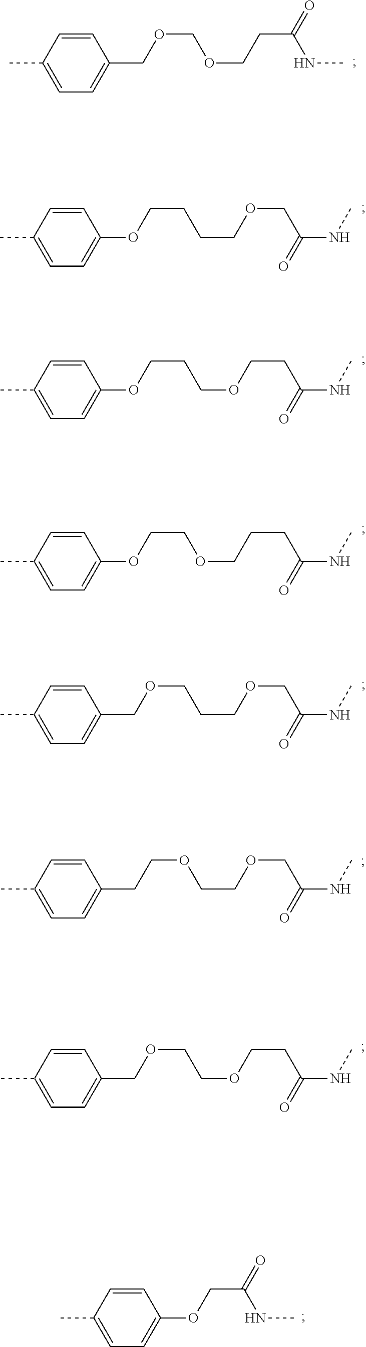

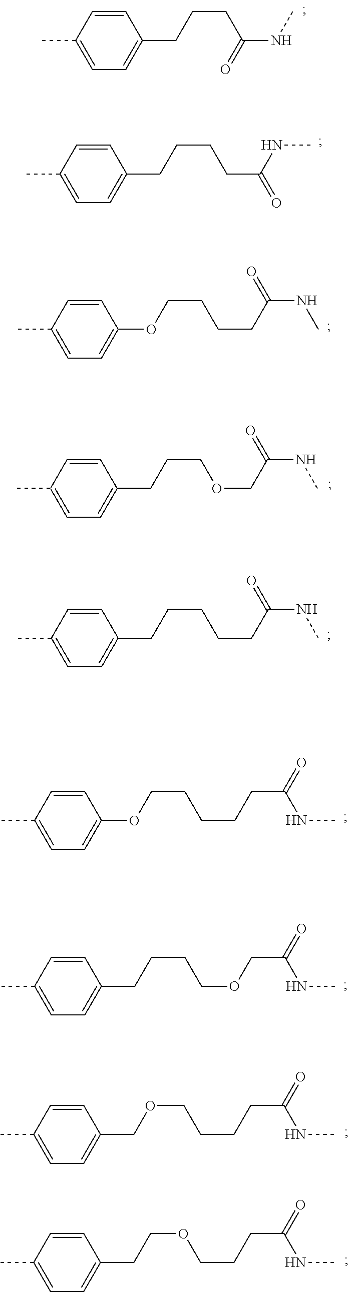

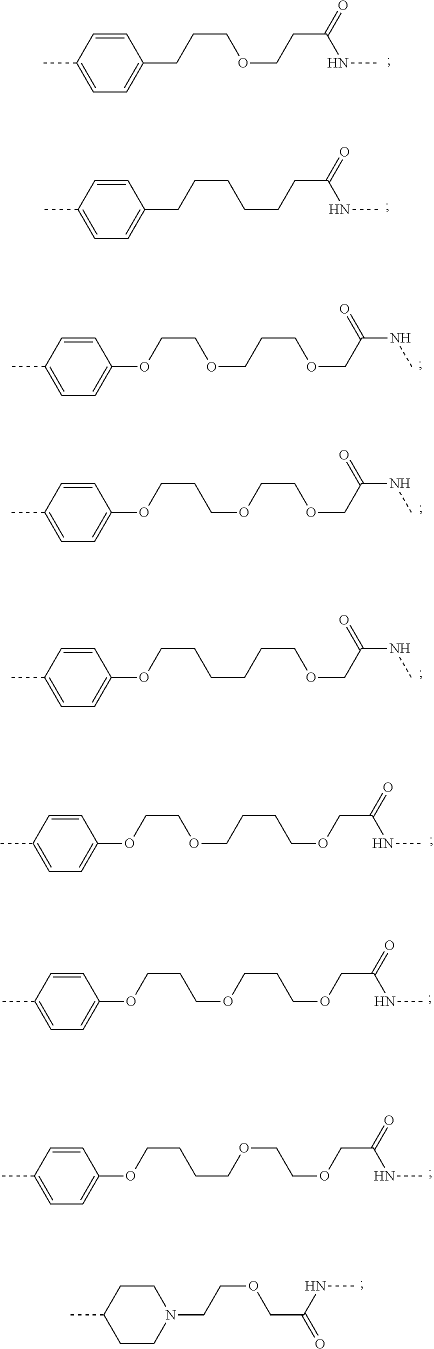

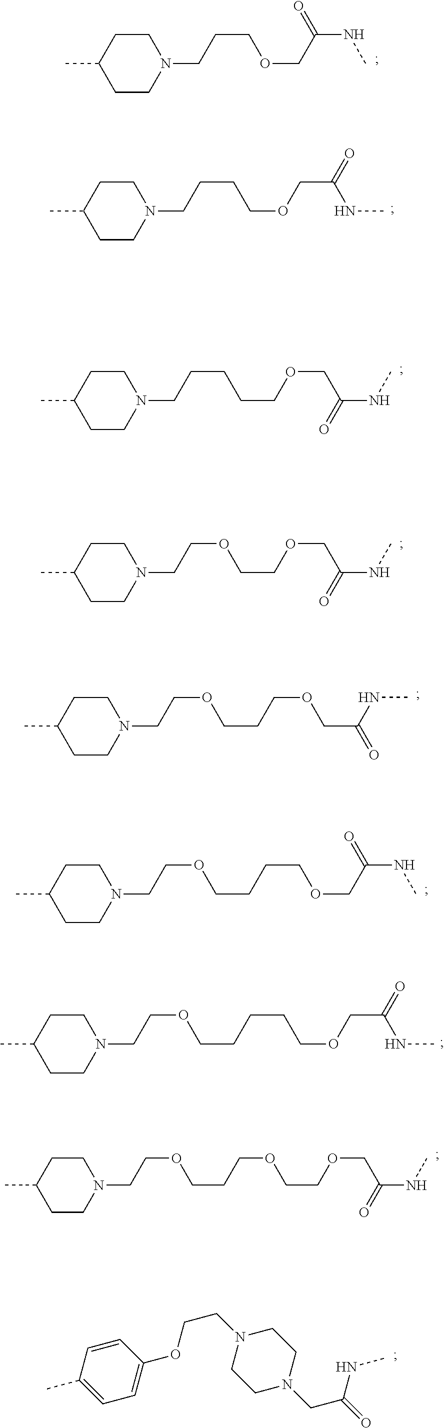

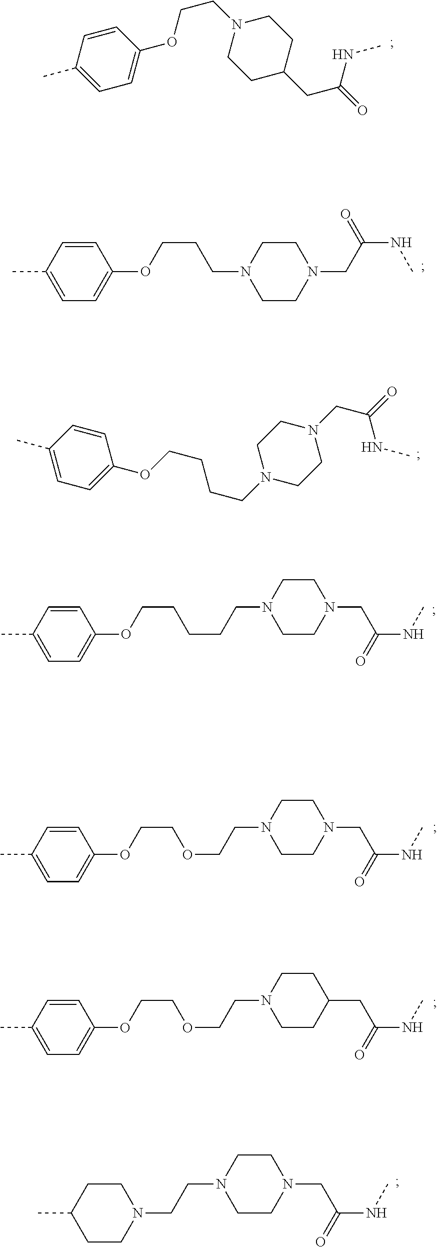

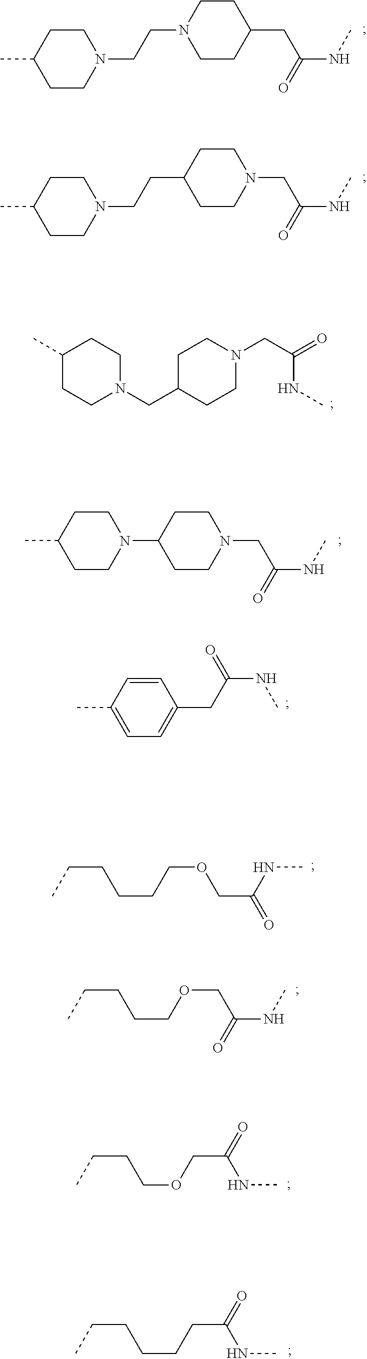

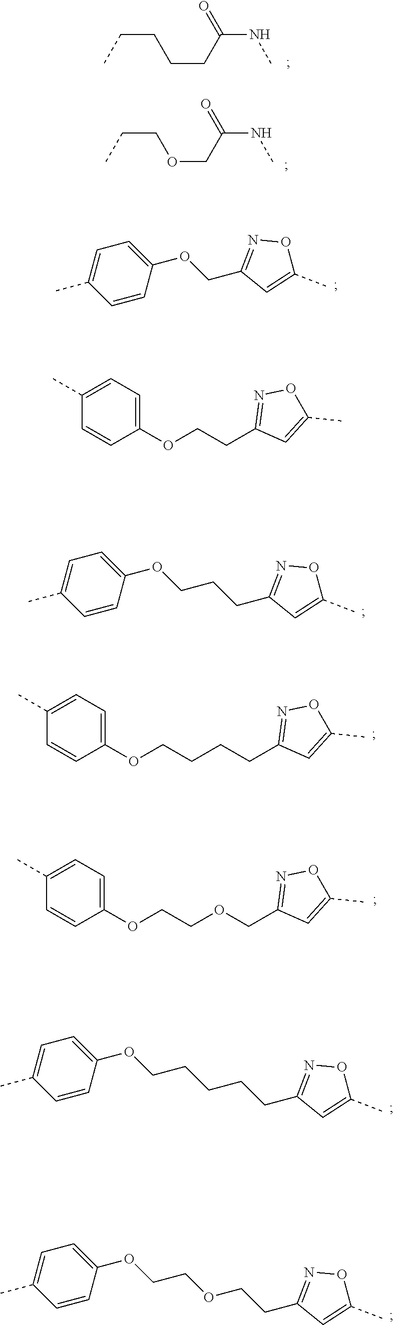

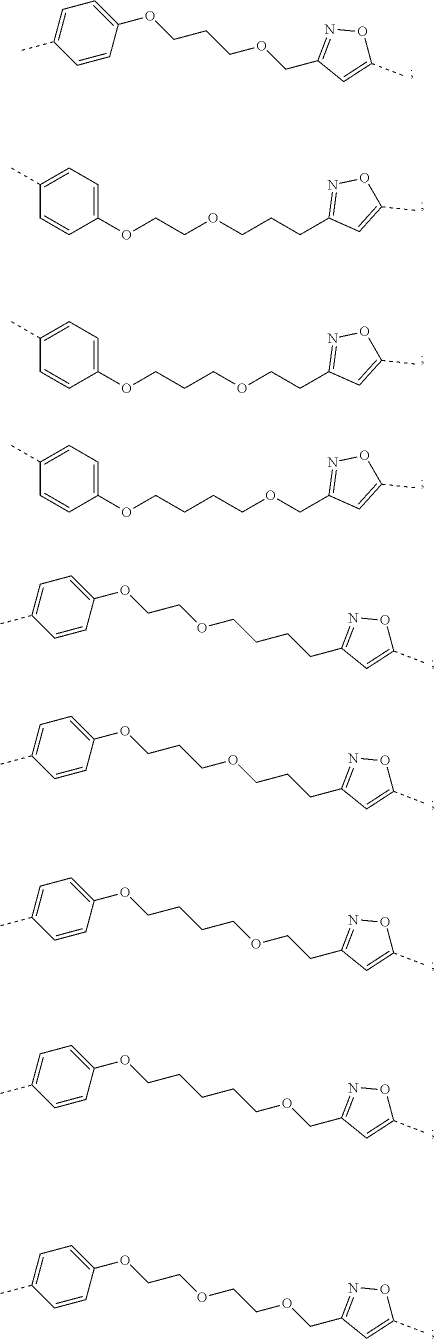

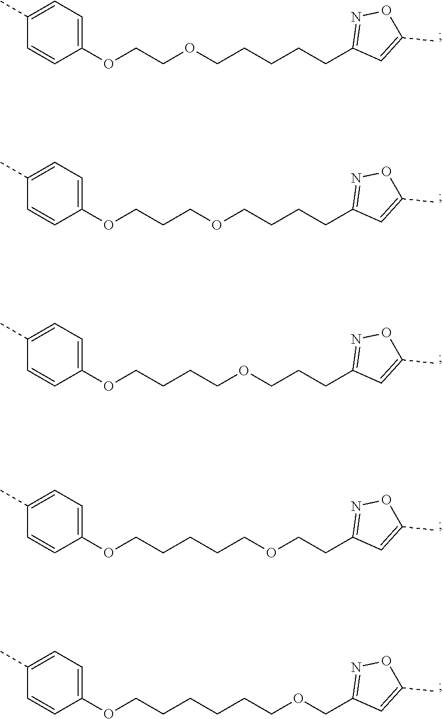

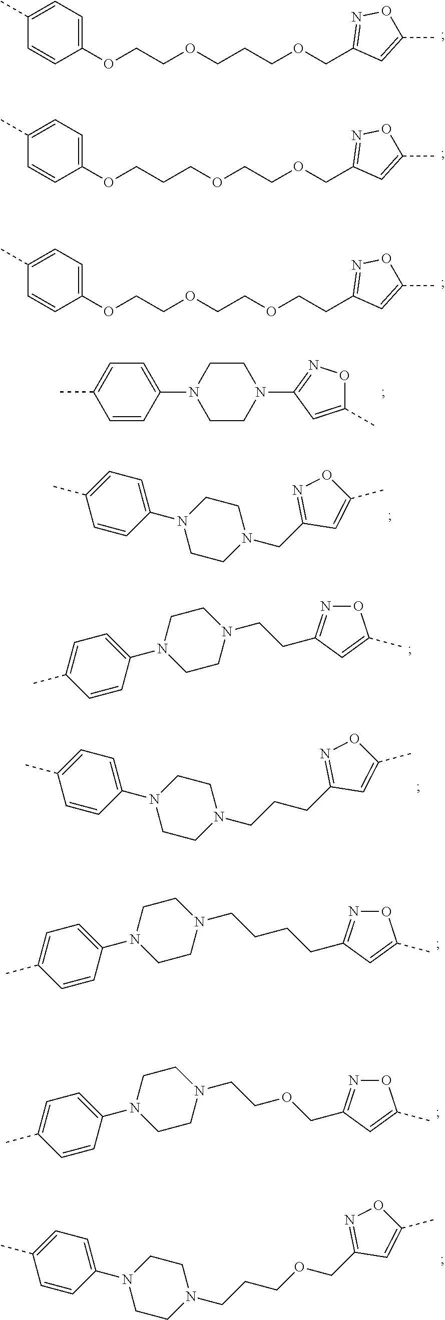

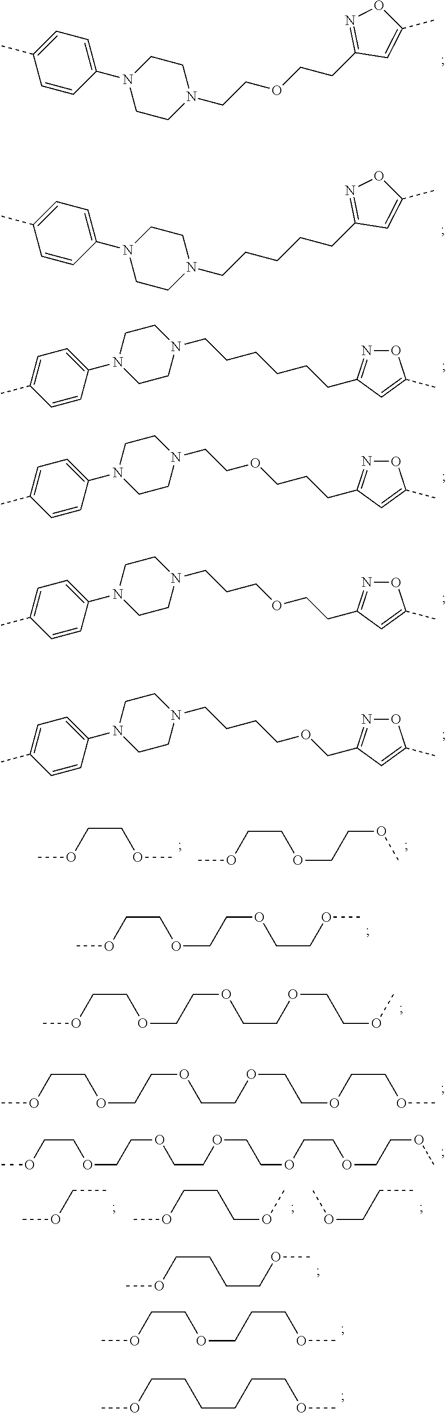

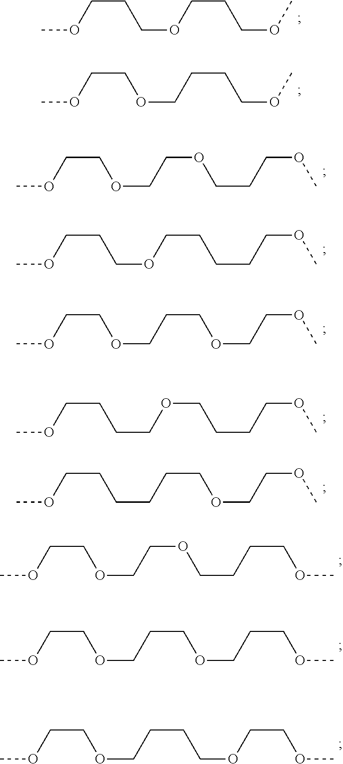

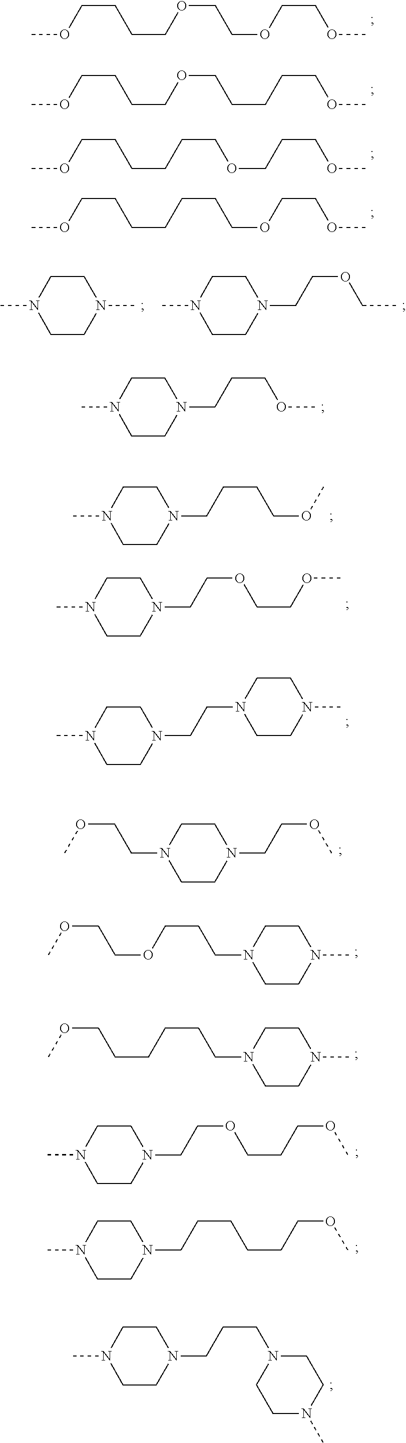

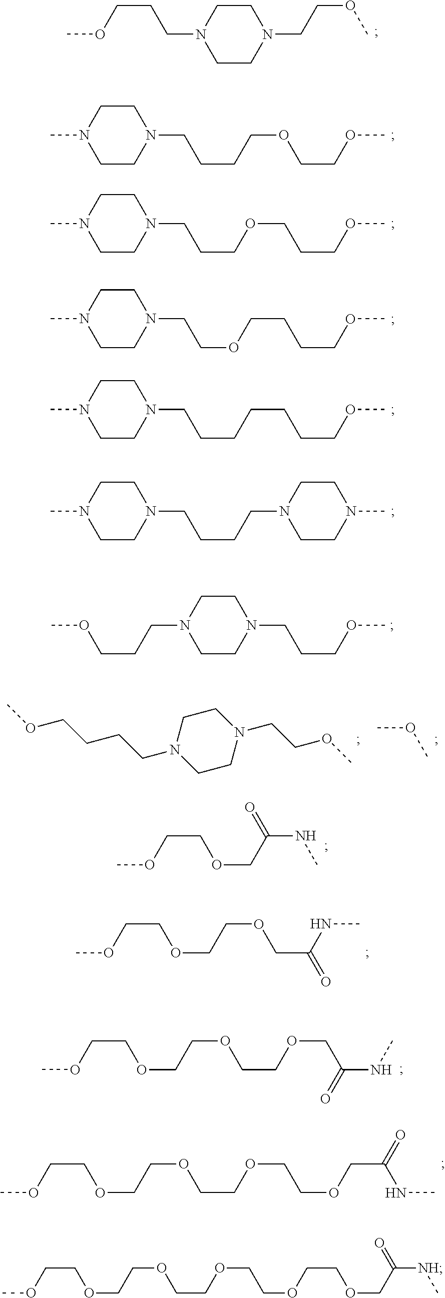

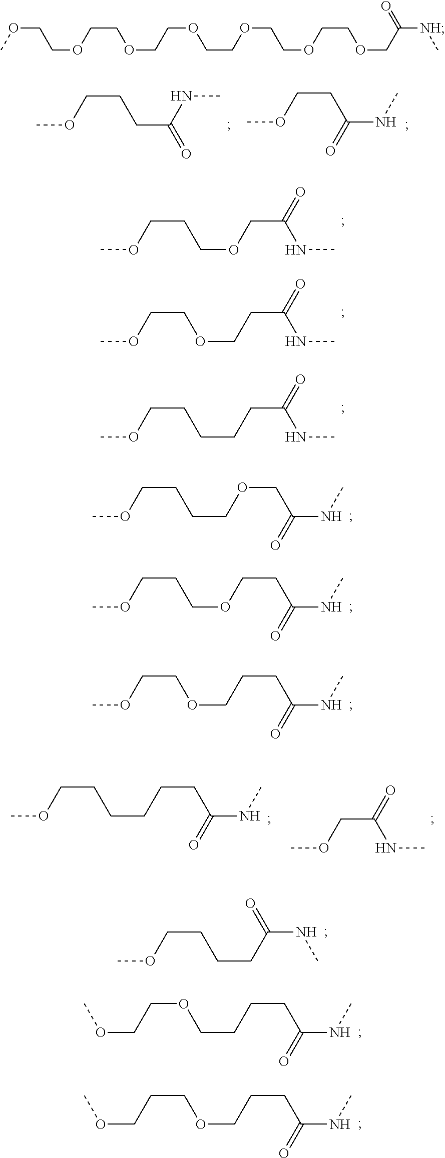

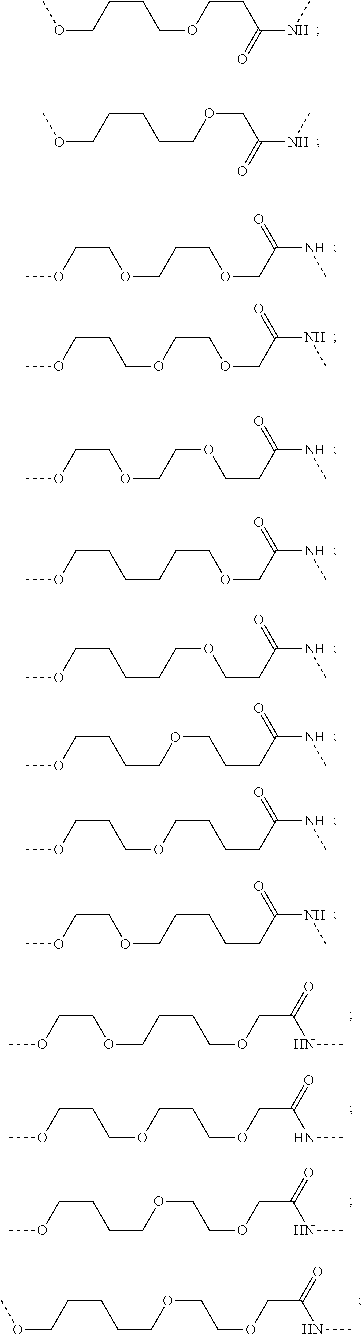

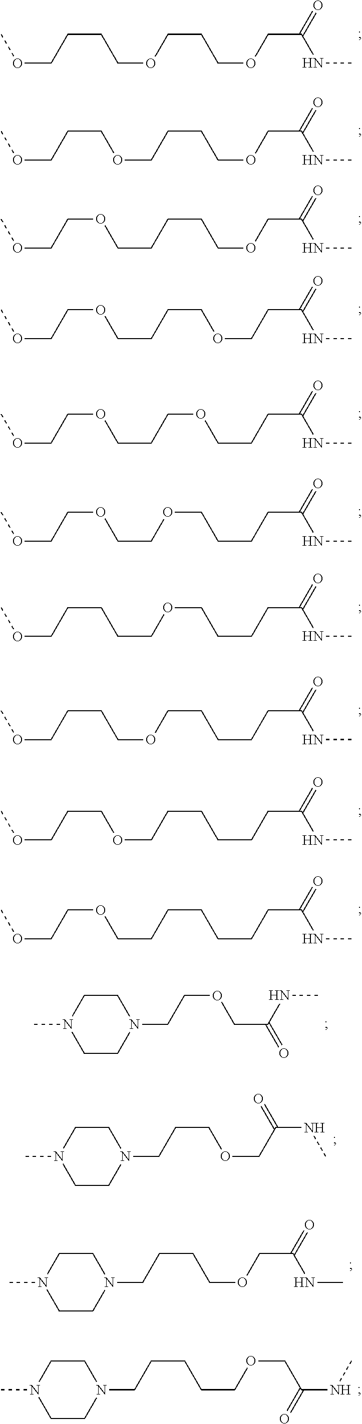

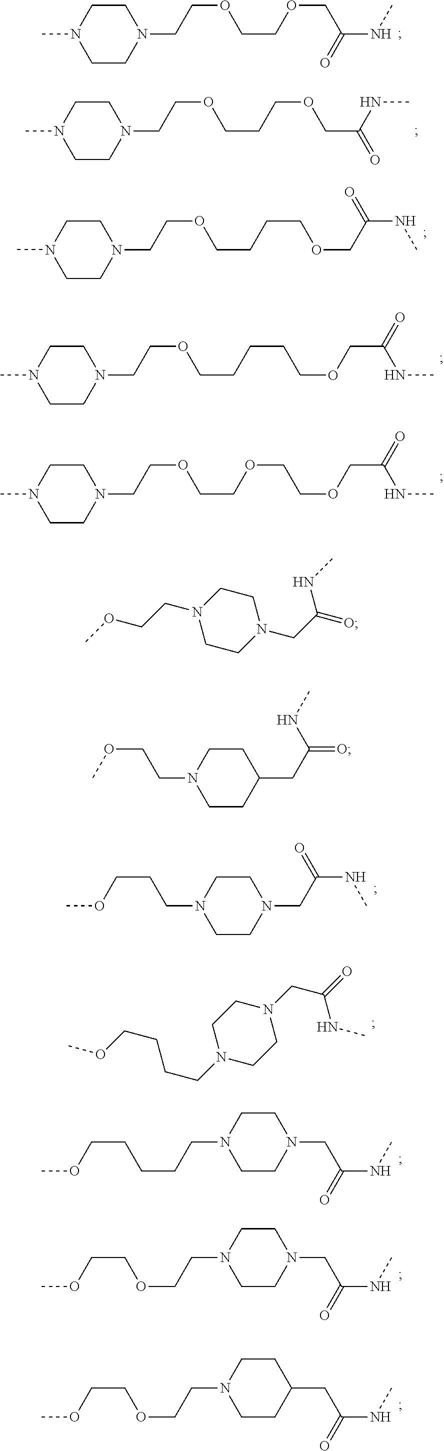

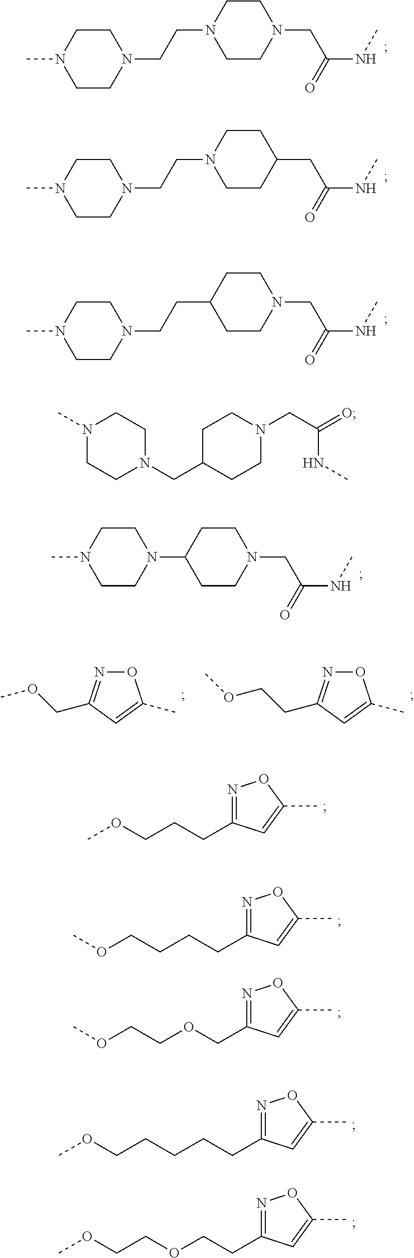

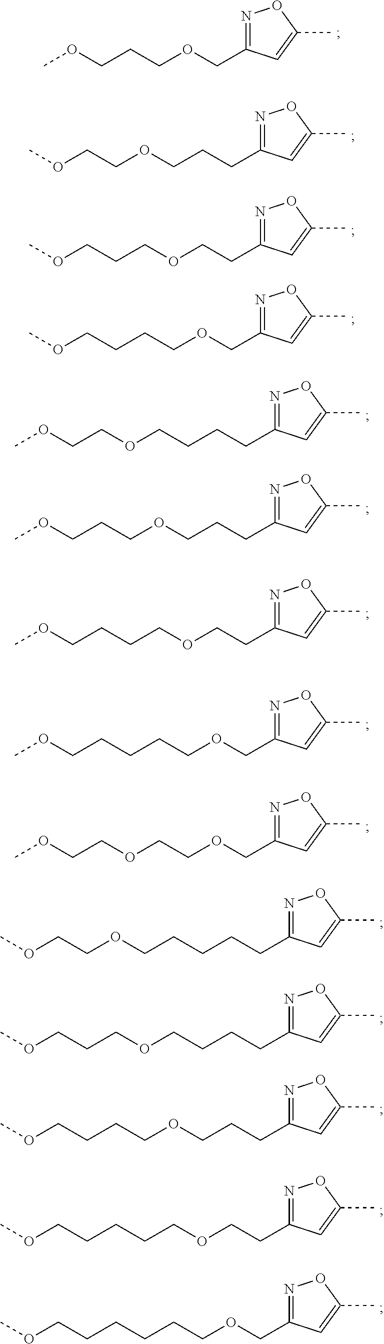

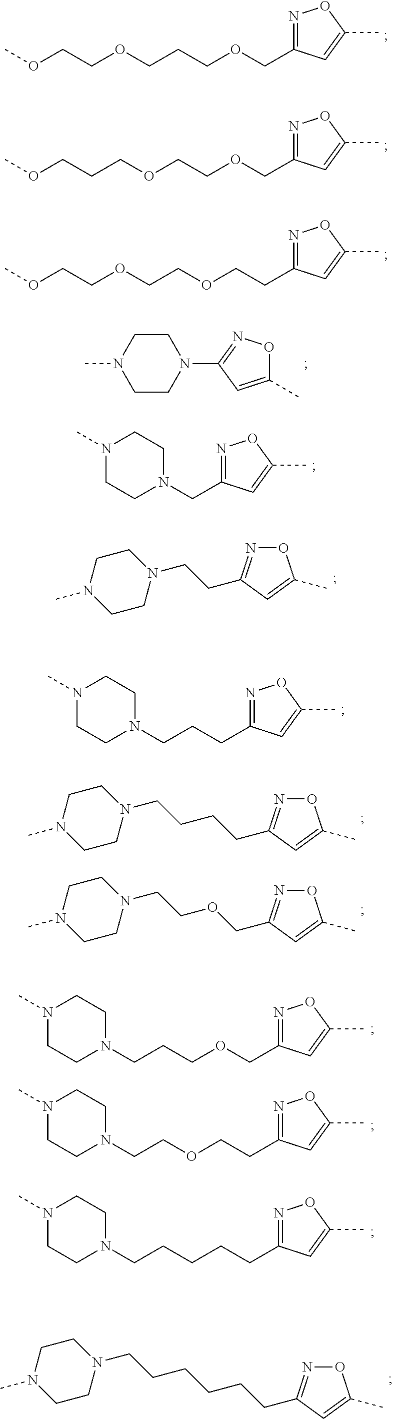

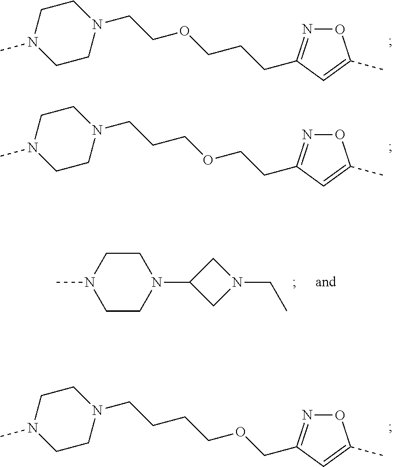

wherein a dashed bond indicates a point of attachment.

In certain embodiments, L has the formula-(C₀₋₁₂ alkylene)-(optionally substituted 3-40 membered heteroalkylene)-(C₀₋₁₂ alkylene)-. In certain embodiments, L is C₄₋₁₄ alkylene. In certain embodiments, L is —(CH₂)₆₋₁₀—.

In certain embodiments, L is —CH₂CH₂ (OCH₂CH₂)—***, —CH₂CH₂ (OCH₂CH₂)₂—***, —CH₂CH₂ (OCH₂CH₂)₃—***, —CH₂CH₂ (OCH₂CH₂)₄—***, —CH₂CH₂ (OCH₂CH₂)₅—***, —CH₂CH₂ (OCH₂CH₂)₆—***, —CH₂CH₂ (OCH₂CH₂)₇—***, —CH₂CH₂ (OCH₂CH₂)₈—***, —CH₂CH₂ (OCH₂CH₂)₉—***, —CH₂CH₂ (OCH₂CH₂)₁₀—***, —CH₂CH₂ (OCH₂CH₂)₁₁—***, —CH₂CH₂ (OCH₂CH₂)₁₂—***, —CH₂CH₂ (OCH₂CH₂)₁₃—***, —CH₂CH₂ (OCH₂CH₂)₁₄—***, —CH₂CH₂ (OCH₂CH₂)₁₅—***, or —CH₂CH₂ (OCH₂CH₂)₁₆-20-***, where *** is a point of attachment to TPL.

In certain embodiments, L is —(C₂₋₂₀ alkylene)-(OCH₂CH₂)₂₋₄—(C₀₋₄ alkylene)-***, —(C₂₋₂₀ alkylene)-(OCH₂CH₂)₅₋₇—(C₀₋₄ alkylene)-***, —(C₂₋₂₀ alkylene)-(OCH₂CH₂)₈₋₁₀—(C₀₋₄ alkylene)-***, —(C₂₋₂₀ alkylene)-(OCH₂CH₂)₁₁₋₁₃—(C₀₋₄ alkylene)-***, —(C₂₋₂₀ alkylene)-(OCH₂CH₂)₁₄₋₁₆—(C₀₋₄ alkylene)-***, —(C₂₋₂₀ alkylene)-(OCH₂CH₂)₁₇₋₂₀—(C₀₋₄ alkylene)-***, —(C₁₋₂₀ alkylene)-(OCH₂CH₂)₁₋₁₀—(C₀₋₄ alkylene)-C(O)—***, or —(C₁₋₂₀ alkylene)-(OCH₂CH₂)₁₁₋₂₀—(C₀₋₄ alkylene)-C(O)—***, where *** is a point of attachment to TPL.

In certain embodiments, L is —O (CH₂CH₂O)₂₋₄—(C₀₋₄ alkylene)-***, —O (CH₂CH₂O)₅₋₇—(C₀₋₄ alkylene)-***, —O (CH₂CH₂O)₈₋₁₀—(C₀₋₄ alkylene)-***, —O (CH₂CH₂O)₁₁₋₁₃—(C₀₋₄ alkylene)-***, —O (CH₂CH₂O)₁₄₋₁₆—(C₀₋₄ alkylene)-***, —O (CH₂CH₂O)₁₆₋₂₀—(C₀₋₄ alkylene)-***, —O (CH₂CH₂O)₂₋₁₀—(C₀₋₄ alkylene)C(O)—***, or —O (CH₂CH₂O)₁₁₋₂₀—(C₀₋₄ alkylene)C(O)—***, where *** is a point of attachment to TPL.

In certain embodiments, L is —(C₀₋₂₀ alkylene)-(OCH₂CH₂)₁₋₁₀—(N(C₁₋₄ alkyl))-***, —(C₀₋₂₀ alkylene)-(OCH₂CH₂)₁₁₋₂₀—(N(C₁₋₄ alkyl))-***, —(C₀₋₂₀ alkylene)-(CH₂CH₂O)₁₋₁₀—(C₂₋₁₀ alkylene)-(N(C₁₋₄ alkyl))-(C₀₋₁₀ alkylene)-***, or —(C₀₋₂₀ alkylene)-(CH₂CH₂O)₁₁₋₂₀—(C₂₋₁₀ alkylene)-(N(C₁₋₄ alkyl))-(C₀₋₁₀ alkylene)-***, where *** is a point of attachment to TPL.

In certain embodiments, L is —(C₂₋₁₀ alkylene)-(OCH₂CH₂)₂₋₄—O-(3-6 membered saturated heterocyclylene containing 1 or 2 heteroatoms independently selected from nitrogen and oxygen)-(C₁₋₅ alkylene)-***, —(C₂₋₁₀ alkylene)-(3-6 membered saturated heterocyclylene containing 1 or 2 heteroatoms independently selected from nitrogen and oxygen)-(C₁₋₅ alkylene)-***, —(C₂₋₁₀ alkylene)-N(H)—(C₁₋₅ alkylene)-***, —(C₂₋₁₀ alkylene)-N(C₁₋₆ alkyl)-(C₁₋₅ alkylene)-***, —N(H)—(C₁₋₅ alkylene)-***, —(CH₂CH₂O)₁₋₄—(C₁₋₄ alkylene)-***, —(CH₂CH₂O)₁₋₄—(C₁₋₄ alkylene)-N(C₁₋₆ alkyl)-(C₁₋₆ alkylene)-***, —N(H)—(C₂₋₆ alkylene)-(3-6 membered saturated heterocyclylene containing 1 or 2 heteroatoms independently selected from nitrogen and oxygen)-(C₁₋₅ alkylene)-***, —N(H)—(C₂₋₆ alkylene)-(OCH₂CH₂)₁₋₄—O-(3-6 membered saturated heterocyclylene containing 1 or 2 heteroatoms independently selected from nitrogen and oxygen)-(C₁₋₅ alkylene)-***, —N(H)—[—CH₂CH₂O-]₂₋₆-(C₁₋₆ alkylene)-N(C₁₋₆ alkyl)-(C₁₋₆ alkylene)-***, —(C₁₋₆ alkylene)-(3-6 membered saturated heterocyclylene containing 1 or 2 heteroatoms independently selected from nitrogen and oxygen)-(C₁₋₆ alkylene)-***, —N(H)—(C₂₋₁₀ alkylene)-***, —(C₁₋₆ alkylene)-(3-6 membered saturated heterocyclylene containing 1 or 2 heteroatoms independently selected from nitrogen and oxygen)-(C₁₋₆ alkylene)-N(C₁₋₆ alkyl)-(C₁₋₆ alkylene)-***, —N(H)—[CH₂CH₂—O-]₂₋₆-(C₁₋₆ alkylene)-N(C₁₋₆ alkyl)-(C₁₋₆ alkylene)-***, —N(H)—[CH₂CH₂—O-]₂₋₁₀-(C₁₋₆ alkylene)-***, —N(H)—[CH₂CH₂—O-]₂₋₆-(C₁₋₆ alkylene)-N(C₁₋₆ alkyl)C(O)—(C₁₋₆ alkylene)-***, —[CH₂CH₂—O-]₁₋₆-(C₁₋₆ alkylene)-N(C₁₋₆ alkyl)-***, —[CH₂CH₂—O-]₁₋₆-(C₁₋₆ alkylene)-N(H)—***, or —(C₂₋₁₀ alkylene)-(OCH₂CH₂)₂₋₆—(C₃₋₆ cycloalkylene)-***, where *** is a point of attachment to TPL.

In certain embodiments, L is selected from those depicted in the compounds in Table 3, below. In certain embodiments, L is selected from those depicted in the compounds in Table 1 or 1-A, below.

### Exemplary Specific Compounds

In certain embodiments, the compound is a compound in Table 1, or a pharmaceutically acceptable salt thereof. In certain embodiments, the compound is a compound in Table 1. In certain embodiments, the compound is a compound in Table 1-A, or a pharmaceutically acceptable salt thereof. In certain embodiments, the compound is a compound in Table 1-A.

TABLE 1
Compound No. from Working Examples
I-1
I-2
I-3
I-4
I-5
I-6
I-7
I-8
I-9
I-10
I-11
I-12
I-13
I-14
I-15
I-16
I-17
I-18
I-19
I-20
I-21
I-22
I-23
I-24
I-25
I-26
I-27
I-28
I-29
I-30
I-31
I-32
I-33
I-34
I-35
I-36
I-37
I-38
I-39
I-40
I-41
I-42
I-43
I-44
I-45
I-46
I-47
I-48
I-49
I-50
I-51
I-52
I-53
I-54
I-55
I-56
I-57
I-58
I-59
I-60
I-61

```
TABLE 1-A
Compound No. from Working Examples
I-62
I-63
I-64
I-65
I-66
I-67
I-68
I-69
I-70
I-71
I-72
I-73
I-74
I-75
I-76
I-77
I-78
I-79
I-80
I-81
I-82
I-83
I-84
I-85
I-86
I-87
I-88
I-89
I-90
I-91
I-92
I-93
I-94
I-95
I-96
I-97
I-98
I-99
  I-100
  I-101
  I-102
  I-103
  I-104
  I-105
  I-106
  I-107
  I-108
  I-109
  I-110
  I-111
  I-106
  I-112
  I-113
  I-114
```

### Synthetic Methods

Methods for preparing compounds described herein are illustrated in the following synthetic Schemes. The Schemes are given for the purpose of illustrating the invention, and are not intended to limit the scope or spirit of the invention. Starting materials shown in the Schemes can be obtained from commercial sources or can be prepared based on procedures described in the literature.

In the Schemes, it is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated (for example, use of protecting groups or alternative reactions). Protecting group chemistry and strategy is well known in the art, for example, as described in detail in “Protecting Groups in Organic Synthesis”, T. W. Greene and P. G. M. Wuts, 3^{rd} edition, John Wiley & Sons, 1999, the entire contents of which are hereby incorporated by reference.

The synthetic route illustrated in Scheme 1 is a general method for preparing heterobifunctional compounds D. Coupling compound A (a precursor of TPL, for example, a discrete compound that is a target protein ligand) with L′ (a precursor to linker L, containing functionality for coupling to the precursors of both TPL and EPL) affords intermediate B (wherein L″ is a precursor to linker L that contains functionality for coupling to the EPL precursor). Coupling intermediate B with compound C (a precursor of EPL) affords heterobifunctional compound D. Alternatively, the order of coupling compounds A and C to L′ may be reversed, such that L′ is first coupled with compound C, before being coupled to compound A.

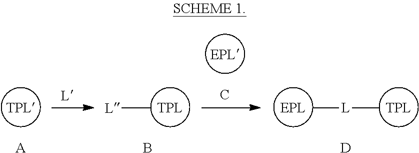

The coupling of compound A with L′, and the coupling of intermediate B with compound C, can be accomplished with a wide variety of strategies. For example, amide coupling conditions can be employed when compound A (or compound C) is to be attached at a modifiable nitrogen atom and L′ (or L″) contains a carboxylic acid group, or vice versa (i.e. compound A contains a carboxylic acid group and L′ contains a nucleophilic amine nitrogen atom). Alternatively, reductive amination conditions can be employed when compound A (or compound C) is to be attached at a modifiable nitrogen atom and L′ (or L″) contains an aldehyde group, or vice versa. Alternatively, nucleophilic substitution conditions can be employed when compound A (or compound C) is to be attached at a modifiable oxygen, nitrogen, or sulfur atom and L′ (or L″) contains a leaving group (such as an alkyl triflate, «-bromoketone, or aryl chloride), or vice versa. As yet another option, transition-metal-mediated coupling conditions can be employed when compound A (or compound C) is to be attached at a modifiable carbon, oxygen, or nitrogen atom (where the carbon atom may be activated, for example, with a bromide or sulfonate) and L′ (or L″) contains a suitable coupling partner (for example, an olefin for a Heck coupling, a trialkylstannane for a Stille coupling, or a boronic acid or boronate ester for a Suzuki coupling, Buchwald-Hartwig amination, or Chan-Lam coupling), or vice versa.

It is understood by one skilled in the art of organic synthesis that protecting group strategies may be employed as necessary, for example, if L′ contains two of the same functional group that are to be selectively coupled to compound A and compound C. For example, L′ may contain, for example, both an unprotected carboxylic acid for coupling to compound A, and a carboxylic acid group that is protected (for example, as a methyl or benzyl ester) during the coupling with compound A and subsequently deprotected (for example, via basic hydrolysis of a methyl ester or hydrogenolysis of a benzyl ester) prior to coupling with compound C.

### II. Therapeutic Applications

The heterobifunctional compounds described herein, such as a compound of Formula I, or other compounds in Section I, provide therapeutic benefits to patients suffering from cancer and/or hepatitis. Accordingly, one aspect of the invention provides a method of treating cancer. The method comprises administering to a patient in need thereof a therapeutically effective amount of a compound described herein, such as a compound of Formula I, or other compounds in Section I, to treat the cancer. In certain embodiments, the particular compound of Formula I is a compound defined by one of the embodiments described above.

Another aspect of the invention provides a method of treating hepatitis. The method comprises administering to a patient in need thereof a therapeutically effective amount of a compound described herein, such as a compound of Formula I, or other compounds in Section I, to treat the hepatitis. In certain embodiments, the particular compound of Formula I is a compound defined by one of the embodiments described above.

### Cancer

In certain embodiments, the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct and gallbladder cancers, liver cancer, pancreatic cancer, bladder cancer, urinary tract cancer, kidney cancer, eye cancer, thyroid cancer, lymphoma, or leukemia.

In certain embodiments, the cancer is squamous cell cancer, lung cancer including small cell lung cancer, non-small cell lung cancer, vulval cancer, thyroid cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, anal carcinoma, penile carcinoma, and head and neck cancer. In certain embodiments, the cancer is at least one selected from the group consisting of ALL, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL, Philadelphia chromosome positive CML, lymphoma, leukemia, multiple myeloma myeloproliferative diseases, large B cell lymphoma, or B cell Lymphoma.

In certain embodiments, the cancer is a solid tumor or leukemia. In certain other embodiments, the cancer is colon cancer, pancreatic cancer, breast cancer, ovarian cancer, prostate cancer, squamous cell carcinoma, basal cell carcinoma, adenocarcinoma, sweat gland carcinoma, sebaceous gland carcinoma, lung cancer, leukemia, bladder cancer, stomach cancer, cervical cancer, testicular cancer, skin cancer, rectal cancer, thyroid cancer, kidney cancer, uterus cancer, esophagus cancer, liver cancer, an acoustic neuroma, oligodendroglioma, meningioma, neuroblastoma, or retinoblastoma. In certain other embodiments, the cancer is small cell lung cancer, non-small cell lung cancer, melanoma, cancer of the central nervous system tissue, brain cancer, Hodgkin's lymphoma, non-Hodgkin's lymphoma, cutaneous T-Cell lymphoma, cutaneous B-Cell lymphoma, or diffuse large B-Cell lymphoma. In certain other embodiments, the cancer is breast cancer, colon cancer, small-cell lung cancer, non-small cell lung cancer, prostate cancer, renal cancer, ovarian cancer, leukemia, melanoma, or cancer of the central nervous system tissue. In certain other embodiments, the cancer is colon cancer, small-cell lung cancer, non-small cell lung cancer, renal cancer, ovarian cancer, renal cancer, or melanoma.

In certain embodiments, the cancer is a fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, epithelial carcinoma, glioma, astrocytoma, medulloblastoma, or hemangioblastoma.

In certain embodiments, the cancer is a neuroblastoma, meningioma, hemangiopericytoma, multiple brain metastase, glioblastoma multiforms, glioblastoma, brain stem glioma, poor prognosis malignant brain tumor, malignant glioma, anaplastic astrocytoma, anaplastic oligodendroglioma, neuroendocrine tumor, rectal adeno carcinoma, Dukes C & D colorectal cancer, unresectable colorectal carcinoma, metastatic hepatocellular carcinoma, Kaposi's sarcoma, karyotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma, localized melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unresectable hepatocellular carcinoma, Waidenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, or leiomyoma.

In certain embodiments, the cancer is bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In certain embodiments, the cancer is hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis -1 associated malignant peripheral nerve sheath tumors (MPNST); Waldenstrom's macroglobulinemia; or medulloblastoma.

In certain embodiments, the cancer is hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis -1 associated malignant peripheral nerve sheath tumors (MPNST), Waldenstrom's macroglobulinemia, or medulloblastoma.

In certain embodiments, the cancer is a solid tumor, such as a sarcoma, carcinoma, or lymphoma. In certain embodiments, the cancer is kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis -1 associated malignant peripheral nerve sheath tumors (MPNST); Waldenstrom's macroglobulinemia; or medulloblastoma.

In certain embodiments, the cancer is renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis -1 associated malignant peripheral nerve sheath tumors (MPNST), Waldenstrom's macroglobulinemia, or medulloblastoma.

In certain embodiments, the cancer is hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis -1 associated malignant peripheral nerve sheath tumors (MPNST), Waldenstrom's macroglobulinemia, or medulloblastoma.

In certain embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis -1 associated MPNST. In some embodiments, the cancer is Waldenstrom's macroglobulinemia. In some embodiments, the cancer is medulloblastoma.

### Hepatitis

In certain embodiments, the disease to be treated is hepatitis. In certain embodiments, the hepatitis is hepatitis A, B, or C.

### Causing Death of Cancer Cell

Another aspect of the invention provides a method of causing death of a cancer cell. The method comprises contacting a cancer cell with an effective amount of a compound described herein, such as a compound of Formula I or II, or other compounds in Section I, to cause death of the cancer cell. In certain embodiments, the particular compound of Formula I or II is a compound defined by one of the embodiments described above.

In certain embodiments, the cancer cell is selected from ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct and gallbladder cancers, liver cancer, pancreatic cancer, bladder cancer, urinary tract cancer, kidney cancer, eye cancer, thyroid cancer, lymphoma, or leukemia. In certain embodiments, the cancer cell is one or more of the cancers recited in the section above entitled “Cancer.”

### Combination Therapies

The compounds useful within the methods of the invention may be used in combination with one or more additional therapeutic agents useful for treating any disease contemplated herein. These additional therapeutic agents may comprise compounds that are commercially available or synthetically accessible to those skilled in the art. These additional therapeutic agents are known to treat, prevent, or reduce the symptoms, of a disease or disorder contemplated herein. Accordingly, in certain embodiments, the method further comprises administering to the subject an additional therapeutic agent that treats the disease contemplated herein.

In certain embodiments, administering the compound of the invention to the subject allows for administering a lower dose of the additional therapeutic agent as compared to the dose of the additional therapeutic agent alone that is required to achieve similar results in treating the disease contemplated herein. For example, in certain embodiments, the compound of the invention enhances the therapeutic activity of the additional therapeutic compound, thereby allowing for a lower dose of the additional therapeutic compound to provide the same effect.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

In certain embodiments, the compound of the invention and the therapeutic agent are co-administered to the subject. In other embodiments, the compound of the invention and the therapeutic agent are coformulated and co-administered to the subject.

In certain embodiments, the compound is administered in combination with a second therapeutic agent having activity against cancer. In certain embodiments, the second therapeutic agent is mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor -1, colony stimulating factor -2, denileukin diftitox, interleukin-2, and leutinizing hormone releasing factor.

In certain embodiments, the second therapeutic agent is an mTOR inhibitor, which inhibits cell proliferation, angiogenesis and glucose uptake. Approved mTOR inhibitors useful in the present invention include everolimus (Afinitor®, Novartis); temsirolimus (Torisel®, Pfizer); and sirolimus (Rapamune®, Pfizer).

In certain embodiments, the second therapeutic agent is a Poly ADP ribose polymerase (PARP) inhibitor. Approved PARP inhibitors useful in the present invention include olaparib (Lynparza®, AstraZeneca); rucaparib (Rubraca®, Clovis Oncology); and niraparib (Zejula®, Tesaro). Other PARP inhibitors being studied which may be used in the present invention include talazoparib (MDV3800/BMN 673/LT00673, Medivation/Pfizer/Biomarin); veliparib (ABT-888, AbbVie); and BGB-290 (BeiGene, Inc.).

In certain embodiments, the second therapeutic agent is a phosphatidylinositol 3 kinase (PI3K) inhibitor. Approved PI3K inhibitors useful in the present invention include idelalisib (Zydelig®, Gilead). Other PI3K inhibitors being studied which may be used in the present invention include alpelisib (BYL719, Novartis); taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

In certain embodiments, the second therapeutic agent is a proteasome inhibitor. Approved proteasome inhibitors useful in the present invention include bortezomib (Velcade®, Takeda); carfilzomib (Kyprolis®, Amgen); and ixazomib (Ninlaro®, Takeda).

In certain embodiments, the second therapeutic agent is a histone deacetylase (HDAC) inhibitor. Approved HDAC inhibitors useful in the present invention include vorinostat (Zolinza®, Merck); romidepsin (Istodax®, Celgene); panobinostat (Farydak®, Novartis); and belinostat (Beleodaq®, Spectrum Pharmaceuticals). Other HDAC inhibitors being studied which may be used in the present invention include entinostat (SNDX-275, Syndax Pharmaceuticals)(NCT00866333); and chidamide (Epidaza®, HBI-8000, Chipscreen Biosciences, China).

In certain embodiments, the second therapeutic agent is a CDK inhibitor, such as a CDK 4/6 inhibitor. Approved CDK 4/6 inhibitors useful in the present invention include palbociclib (Ibrance®, Pfizer); and ribociclib (Kisqali®, Novartis). Other CDK 4/6 inhibitors being studied which may be used in the present invention include abemaciclib (Ly2835219, Eli Lilly); and trilaciclib (GIT28, G1 Therapeutics).

In certain embodiments, the second therapeutic agent is an indoleamine (2,3)-dioxygenase (IDO) inhibitor. IDO inhibitors being studied which may be used in the present invention include epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS: F001287 (Bristol-Myers Squibb); Phy906/KD108 (Phytoceutica); and an enzyme that breaks down kynurenine (Kynase, Kyn Therapeutics).

In certain embodiments, the second therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

In certain embodiments, the second therapeutic agent is an aromatase inhibitor. Approved aromatase inhibitors which may be used in the present invention include exemestane (Aromasin®, Pfizer); anastazole (Arimidex®, AstraZeneca) and letrozole (Femara®, Novartis).

In certain embodiments, the second therapeutic agent is an antagonist of the hedgehog pathway. Approved hedgehog pathway inhibitors which may be used in the present invention include sonidegib (Odomzo®, Sun Pharmaceuticals); and vismodegib (Erivedge®, Genentech), both for treatment of basal cell carcinoma.

In certain embodiments, the second therapeutic agent is a folic acid inhibitor. Approved folic acid inhibitors useful in the present invention include pemetrexed (Alimta®, Eli Lilly).

In certain embodiments, the second therapeutic agent is a CC chemokine receptor 4 (CCR4) inhibitor. CCR4 inhibitors being studied that may be useful in the present invention include mogamulizumab (Poteligeo®, Kyowa Hakko Kirin, Japan).

In certain embodiments, the second therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In certain embodiments, the second therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In certain embodiments, the second therapeutic agent is a glutaminase inhibitor. Glutaminase inhibitors being studied which may be used in the present invention include CB-839 (Calithera Biosciences).

In certain embodiments, the second therapeutic agent is an antibody that binds to tumor antigens, that is, proteins expressed on the cell surface of tumor cells. Approved antibodies that bind to tumor antigens which may be used in the present invention include rituximab (Rituxan®, Genentech/BiogenIdec); ofatumumab (anti-CD20, Arzerra®, GlaxoSmithKline); obinutuzumab (anti-CD20, Gazyva®, Genentech), ibritumomab (anti-CD20 and Yttrium-90, Zevalin®, Spectrum Pharmaceuticals); daratumumab (anti-CD38, Darzalex®, Janssen Biotech), dinutuximab (anti-glycolipid GD2, Unituxin®, United Therapeutics); trastuzumab (anti-HER2, Herceptin®, Genentech); ado-trastuzumab emtansine (anti-HER2, fused to emtansine, Kadcyla®, Genentech); and pertuzumab (anti-HER2, Perjeta®, Genentech); and brentuximab vedotin (anti-CD30-drug conjugate, Adcetris®, Seattle Genetics).

In certain embodiments, the second therapeutic agent is a topoisomerase inhibitor. Approved topoisomerase inhibitors useful in the present invention include irinotecan (Onivyde®, Merrimack Pharmaceuticals); topotecan (Hycamtin®, GlaxoSmithKline). Topoisomerase inhibitors being studied which may be used in the present invention include pixantrone (Pixuvri®, CTI Biopharma).

In certain embodiments, the second therapeutic agent is a nucleoside inhibitor, or other therapeutic that interfere with normal DNA synthesis, protein synthesis, cell replication, or will otherwise inhibit rapidly proliferating cells. Such nucleoside inhibitors or other therapeutics include trabectedin (guanidine alkylating agent, Yondelis®, Janssen Oncology), mechlorethamine (alkylating agent, Valchlor®, Aktelion Pharmaceuticals); vincristine (Oncovin®, Eli Lilly; Vincasar®, Teva Pharmaceuticals; Marqibo®, Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CeeNUR, Bristol-Myers Squibb; Gleostine®, NextSource Biotechnology); azacitidine (pyrimidine nucleoside analog of cytidine, Vidaza®, Celgene); omacetaxine mepesuccinate (cephalotaxine ester)(protein synthesis inhibitor, Synribo®; Teva Pharmaceuticals); asparaginase Erwinia chrysanthemi (enzyme for depletion of asparagine, Elspar®, Lundbeck; Erwinaze®, EUSA Pharma); eribulin mesylate (microtubule inhibitor, tubulin-based antimitotic, Halaven®, Eisai); cabazitaxel (microtubule inhibitor, tubulin-based antimitotic, Jevtana®, Sanofi-Aventis); capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic, Ixempra®, Bristol-Myers Squibb); nelarabine (prodrug of deoxyguanosine analog, nucleoside metabolic inhibitor, Arranon®, Novartis); clorafabine (prodrug of ribonucleotide reductase inhibitor, competitive inhibitor of deoxycytidine, Clolar®, Sanofi-Aventis); and trifluridine and tipiracil (thymidine-based nucleoside analog and thymidine phosphorylase inhibitor, Lonsurf®, Taiho Oncology).

In certain embodiments, the second therapeutic agent is a platinum-based therapeutic, also referred to as platins. Platins cause cross-linking of DNA, such that they inhibit DNA repair and/or DNA synthesis, mostly in rapidly reproducing cells, such as cancer cells. Approved platinum-based therapeutics which may be used in the present invention include cisplatin (Platinol®, Bristol-Myers Squibb); carboplatin (Paraplatin®, Bristol-Myers Squibb; also, Teva; Pfizer); oxaliplatin (Eloxitin® Sanofi-Aventis); and nedaplatin (Aqupla®, Shionogi). Other platinum-based therapeutics which have undergone clinical testing and may be used in the present invention include picoplatin (Poniard Pharmaceuticals); and satraplatin (JM-216, Agennix).

In certain embodiments, the second therapeutic agent is a taxane compound, which causes disruption of microtubules, which are essential for cell division. Approved taxane compounds which may be used in the present invention include paclitaxel (Taxol®, Bristol-Myers Squibb), docetaxel (Taxotere®, Sanofi-Aventis; Docefrez®, Sun Pharmaceutical), albumin-bound paclitaxel (Abraxane®; Abraxis/Celgene), and cabazitaxel (Jevtana®, Sanofi-Aventis). Other taxane compounds which have undergone clinical testing and may be used in the present invention include SID530 (SK Chemicals, Co.)(NCT00931008).

In certain embodiments, the second therapeutic agent is an inhibitor of anti-apoptotic proteins, such as BCL-2. Approved anti-apoptotics which may be used in the present invention include venetoclax (Venclexta®, AbbVie/Genentech); and blinatumomab (Blincyto®, Amgen). Other therapeutic agents targeting apoptotic proteins which have undergone clinical testing and may be used in the present invention include navitoclax (ABT-263, Abbott), a BCL-2 inhibitor (NCT02079740).

In certain embodiments, the second therapeutic agent is a selective estrogen receptor modulator (SERM), which interferes with the synthesis or activity of estrogens. Approved SERMs useful in the present invention include raloxifene (Evista®, Eli Lilly).

In certain embodiments, the second therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL)(NCT02909972; NCT02264613).

In certain embodiments, the second therapeutic agent is an inhibitor of transforming growth factor -beta (TGF-beta or TGFβ). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA-formerly MSB0011459X), which is a bispecific, anti-PD-L1/TGFβ trap compound (NCT02699515); and (NCT02517398). M7824 is comprised of a fully human IgG1 antibody against PD-L1 fused to the extracellular domain of human TGF-beta receptor II, which functions as a TGFβ “trap.”

In certain embodiments, the second therapeutic agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, the additional therapeutic agent is selected from an oncolytic viral therapy such as pexastimogene devacirepvec (Pexa Vec/JX-594, SillaJen/formerly Jennerex Biotherapeutics), a thymidine kinase-(TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC)(NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONCI (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In certain embodiments, the second therapeutic agent is an immune checkpoint inhibitor selected from a PD-1 antagonist, a PD-L1 antagonist, or a CTLA-4 antagonist. In some embodiments, a compound disclosed herein or a pharmaceutically acceptable salt thereof is administered in combination with nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); or atezolizumab (anti-PD-L1 antibody, Tecentriq®, Genentech). Other immune checkpoint inhibitors suitable for use in the present invention include REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; and PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

Another aspect of the invention provides for the use of a compound described herein (such as a compound of Formula I, Formula II, or other compounds in Section I) in the manufacture of a medicament. In certain embodiments, the medicament is for treating a disease described herein, such as cancer.

Another aspect of the invention provides for the use of a compound described herein (such as a compound of Formula I, Formula II, or other compounds in Section I) for treating a medical disease, such a disease described herein (e.g., cancer).

### III. Pharmaceutical Compositions and Dosing Considerations

As indicated above, the invention provides pharmaceutical compositions, which comprise a therapeutically-effective amount of one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. In certain embodiments, the invention provides a pharmaceutical composition comprising a compound described herein (e.g., a compound of Formula I) and a pharmaceutically acceptable carrier.

The phrase “therapeutically effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate. Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising a heterobifunctional substituted phenylpyrimidinone or related compound described herein in a therapeutically effective amount for the treatment of a medical disorder described herein.

### IV. Medical Kits

Another aspect of this invention is a kit comprising (i) a compound described herein, such as a compound of Formula I, and (ii) instructions for use, such as treating cancer.

### Examples

The invention now being generally described, will be more readily understood by reference to the following examples, which are included merely for purposes of illustration of certain aspects and embodiments of the present invention, and is not intended to limit the invention.

### General Methods

All reactions were carried out under an atmosphere of dry nitrogen or argon. Glassware was oven-dried prior to use. Unless otherwise indicated, common reagents or materials were obtained from commercial sources and used without further purification. N,N-Diisopropy]ethylamine (DIPEA) was obtained anhydrous by distillation over potassium hydroxide. Tetrahydrofuran (THF), Dichloromethane (CH₂Cl₂), and dimethylformamide (DMF) was dried by a PureSolv™ solvent drying system. PTLC refers to preparatory thin layer chromatographic separation. Abbreviations: HFIP (hexafluoroisopropanol), HEPES (4-(2-hydroxyethyl)-1-piperazineethanesulfonic acid. Flash column chromatography was performed using silica gel 60 (230-400 mesh). Analytical thin layer chromatography (TLC) was carried out on Merck silica gel plates with QF-254 indicator and visualized by UV or KMnO₄.

¹H and ¹³C NMR spectra were recorded on an Agilent DD₂ 500 (500 MHz ¹H; 125 MHz ¹³C) or Agilent DD₂ 600 (600 MHz 1H; 150 MHz 13C) or Agilent DD₂ 400 (400 MHZ 1H; 100 MHz 13C) spectrometer at room temperature. Chemical shifts were reported in ppm relative to the residual CDCl₃ (δ 7.26 ppm ¹H; δ 77.0 ppm ¹³C), CD₃OD (δ 3.31 ppm 1H; δ 49.00 ppm ¹³C), or d₆-DMSO (δ 2.50 ppm ¹H; δ 39.52 ppm ¹³C). NMR chemical shifts were expressed in ppm relative to internal solvent peaks, and coupling constants were measured in Hz. (bs=broad signal). In most cases, only peaks of the major rotamer are reported.

Mass spectra were obtained using Agilent 1100 series LC/MSD spectrometers. Analytical HPLC analyses were carried out on 250×4.6 mm C-18 column using gradient conditions (10-100% B, flow rate=1.0 mL/min, 20 min), or as described in the LC-MS Method tables.

Unless indicated otherwise, preparative HPLC was carried out on 250×21.2 mm C-18 column using gradient conditions (10-100% B, flow rate=10.0 mL/min, 20 min). The eluents used were: solvent A (H₂O with 0.1% TFA) and solvent B (CH₃CN with 0.1% TFA). Final products were typically purified via reversed-phase HPLC, PTLC, or flash column chromatography. The abbreviation “TFA” refers to trifluoroacetic acid.

LC-MS Method 01

| Instrument | | Agilent 1100 LC & Agilent G1956A | | |
|---|---|---|---|---|
| Software | | Agilent Chemstation Rev. B. 04.03[54] | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | Gradient | Time(min) | B(%) | Flow(mL/min) |
| | | 0.00 | 1 | 0.8 |
| | | 0.40 | 1 | 0.8 |
| | | 3.40 | 90 | 0.8 |
| | | 3.90 | 100 | 0.8 |
| | | 3.91 | 1 | 0.8 |
| | | 4.00 | 1 | 1.0 |
| | | 4.50 | 1 | 1.0 |

| | | |
|---|---|---|
| | Post time(min) | 0 |
| | Column Temp | 50° C. |
| | Detector | DAD |
| MS | Ionization source | ESI |
| | Drying Gas | N₂ |
| | Drying Gas Flow | 10(L/min) |
| | Nebulizer Pressure | 40(psi) |
| | Drying Gas | 350° C. |
| | Temperature | |
| | Capillary Voltage | 2500(V) |
| | MS Polarity | Positive |
| | MS Mode | Scan |
| | Mass Range | 100-1500 |

LC-MS Method 10

| Instrument | | Agilent 1100 LC & Agilent G1956A | | |
|---|---|---|---|---|
| Software | | Agilent Chemstation Rev. B. 04.03[54] | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | Gradient | Time(min) | B(%) | Flow(mL/min) |
| | | 0.00 | 10 | 0.8 |
| | | 0.40 | 10 | 0.8 |
| | | 3.40 | 100 | 0.8 |
| | | 3.90 | 100 | 0.8 |
| | | 3.91 | 10 | 0.8 |
| | | 4.00 | 10 | 1.0 |
| | | 4.50 | 10 | 1.0 |

| | | |
|---|---|---|
| | Post time(min) | 0 |
| | Column Temp | 50° C. |
| | Detector | DAD |
| MS | Ionization source | ESI |
| | Drying Gas | N₂ |
| | Drying Gas Flow | 10(L/min) |
| | Nebulizer Pressure | 40(psi) |
| | Drying Gas | 350° C. |
| | Temperature | |
| | Capillary Voltage | 2500(V) |
| | MS Polarity | Positive |
| | MS Mode | Scan |
| | Mass Range | 100-1500 |

LC-MS Method 25

| Instrument | | Agilent 1100 LC & Agilent G1956A | | |
|---|---|---|---|---|
| Software | | Agilent Chemstation Rev. B. 04.03[54] | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | Gradient | Time(min) | B(%) | Flow(mL/min) |
| | | 0.00 | 25 | 0.8 |
| | | 0.40 | 25 | 0.8 |
| | | 3.40 | 100 | 0.8 |
| | | 3.90 | 100 | 0.8 |
| | | 3.91 | 25 | 0.8 |
| | | 4.00 | 25 | 1.0 |
| | | 4.50 | 25 | 1.0 |

| | | |
|---|---|---|
| | Post time(min) | 0 |
| | Column Temp | 50° C. |
| | Detector | DAD |
| MS | Ionization source | ESI |
| | Drying Gas | N₂ |
| | Drying Gas Flow | 10(L/min) |
| | Nebulizer Pressure | 40(psi) |
| | Drying Gas | 350° C. |
| | Temperature | |
| | Capillary Voltage | 2500(V) Positive |
| | MS Polarity | Positive |
| | MS Mode | Scan |
| | Mass Range | 100-1500 |

LC-MS METHOD 40

| Instrument | | Agilent 1100 LC & Agilent G1956A | | |
|---|---|---|---|---|
| Software | | Agilent Chemstation Rev. B. 04.03[16] | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | Gradient | Time(min) | B(%) | Flow(mL/min) |
| | | 0.00 | 40 | 0.8 |
| | | 0.40 | 40 | 0.8 |
| | | 3.40 | 100 | 0.8 |
| | | 3.90 | 100 | 0.8 |
| | | 3.91 | 40 | 0.8 |
| | | 4.00 | 40 | 1.0 |
| | | 4.50 | 40 | 1.0 |

| | | |
|---|---|---|
| | Post time(min) | 0 |
| | Column Temp | 50° C. |
| | Detector | DAD(Agilent 1100)/ELSD(Agilent 1260 Infinity) |
| MS | Ionization source | ESI |
| | Drying Gas | N₂ |
| | Drying Gas Flow | 10(L/min) |
| | Nebulizer Pressure | 2070(Torr) |
| | Drying Gas | 350° C. |
| | Temperature | |
| | Capillary Voltage | 2500(V) Positive |

LC-MS METHOD 100

| Instrument | | Agilent 1100 LC & Agilent G1956A | | |
|---|---|---|---|---|
| Software | | Agilent Chemstation Rev. B. 04.03[16] | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | Gradient | Time(min) | B(%) | Flow(mL/min) |
| | | 0.00 | 0 | 0.6 |
| | | 0.40 | 0 | 0.6 |
| | | 3.40 | 80 | 0.6 |
| | | 3.90 | 100 | 0.6 |
| | | 3.91 | 0 | 0.6 |
| | | 4.00 | 0 | 1.0 |
| | | 4.50 | 0 | 1.0 |

| | | |
|---|---|---|
| | Post time(min) | 1.20 |
| | Column Temp | 50° C. |
| | Detector | DAD |
| | Ionization source | ESI |
| | Drying Gas | N2 |
| | Drying Gas Flow | 10(L/min) |
| | Nebulizer Pressure | 2070(Torr) |
| MS | Drying Gas | 350° C. |
| | Temperature | |
| | Capillary Voltage | 2500(V) Positive |
| | MS Polarity | Positive |
| | MS Mode | Scan |
| | Mass Range | 50-1500 |

### Example 1—Synthesis of Compound I-1

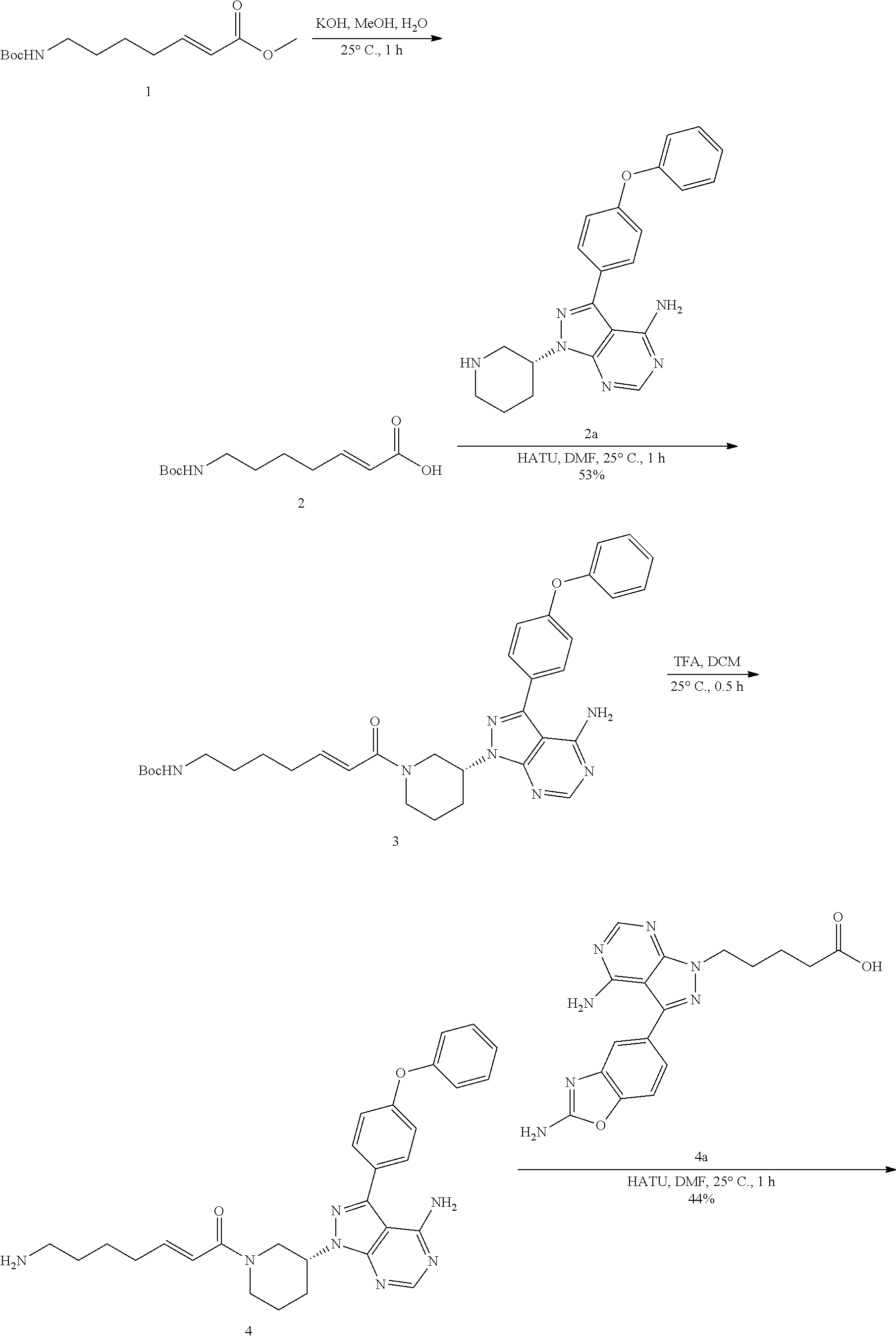 [SMILES: COC(=O)/C=C/CCCCNC(=O)OC(C)(C)C ; Nc1ncnc2c1c(-c1ccc(Oc3ccccc3)cc1)nn2[C@@H]1CCCNC1 ; CC(C)(C)OC(=O)NCCCC/C=C/C(=O)O ; CC(C)(C)OC(=O)NCCCC/C=C/C(=O)N1CCC[C@@H](n2nc(-c3ccc(Oc4ccccc4)cc3)c3c(N)ncnc32)C1 ; NCCCC/C=C/C(=O)N1CCC[C@@H](n2nc(-c3ccc(Oc4ccccc4)cc3)c3c(N)ncnc32)C1 ; Nc1nc2cc(-c3nn(CCCCC(=O)O)c4ncnc(N)c34)ccc2o1]

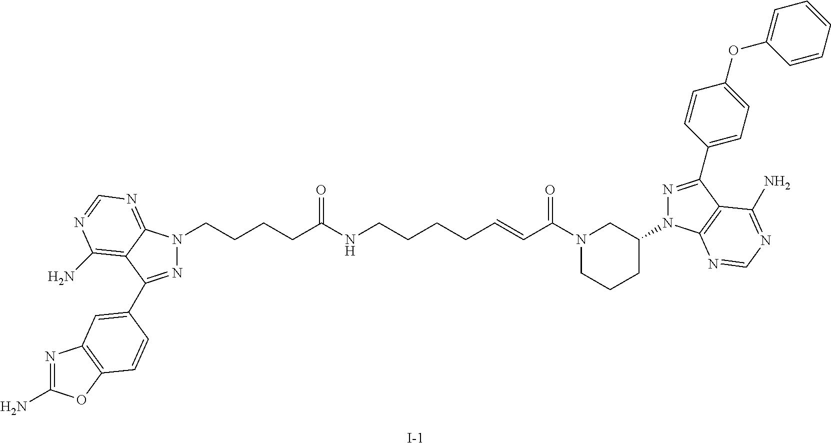 [SMILES: Nc1nc2cc(-c3nn(CCCCC(=O)NCCCC/C=C/C(=O)N4CCC[C@@H](n5nc(-c6ccc(Oc7ccccc7)cc6)c6c(N)ncnc65)C4)c4ncnc(N)c34)ccc2o1 ; [I-]]

### 1.1 Preparation of Compound 2

To a solution of methyl (E)-7-[bis(tert-butoxycarbonyl)amino]hept-2-enoate (400 mg, 1.12 mmol, 1.0 equiv) in MeOH (3 mL) and H₂O (1 mL) was added KOH (81.6 mg, 1.45 mmol, 1.3 equiv). The mixture was stirred at 25° C. for 1 h. The reaction mixture was concentrated under reduced pressure to give the crude product (E)-7-[bis(tert-butoxycarbonyl)amino]hept-2-enoic acid (380 mg, 1.11 mmol) as a white solid. LC-MS: MS (ES⁺): m/z=144.2 [M+H⁺−100].

### 1.2 Preparation of Compound 3

To a solution of (E)-7-[bis(tert-butoxycarbonyl)amino]hept-2-enoic acid (0.38 g, 1.11 mmol, 1.2 equiv) and 3-(4-phenoxyphenyl)-1-[(3R)-3-piperidyl]pyrazolo[3,4-d]pyrimidin-4-amine (0.46 g, 0.92 mmol, 1.0 equiv) in DMF (4 mL) were added HATU (0.46 g, 1.20 mmol, 1.3 equiv) and DIEA (0.36 g, 2.77 mmol, 3.0 equiv). The mixture was stirred at 25° C. for 1 h. The solution was purified by prep-HPLC (column: Waters Xbridge 150*25 mm*5 μm; mobile phase: [water (10 mM NH₄HCO₃)-ACN]; B %: 44%-77%, 9 min) to give tert-butyl-N—[(E)-7-[(3R)-3-[4-amino-3-(4-phenoxyphenyl) pyrazolo[3,4-d]pyrimidin-1-yl]-1-piperidyl]-7-oxo-hept-5-enyl]-N-tert-butoxycarbonyl-carbamate (0.35 g, 0.49 mmol, 53% yield) as a yellow oil. ¹H NMR (400 MHZ, DMSO-d₆): δ 10.54 (d, 1H, J=5.6 Hz), 8.30 (d, 1H, J=2.0 Hz), 7.95 (d, 1H, J=2.0 Hz), 7.64 (d, 2H, J=6.6 Hz), 7.49-7.35 (m, 5H), 7.28-7.19 (m, 2H), 7.17-7.09 (m, 1H), 6.18 (s, 1H), 4.97-4.76 (m, 2H), 4.01-3.89 (m, 2H), 3.60 (dt, 2H, J=6.6, 2.4 Hz), 3.48-3.40 (m, 6H), 3.38-3.36 (m, 2H), 3.24 (td, 3H, J=17.6, 6.0 Hz), 2.98 (s, 2H), 2.79 (s, 1H), 1.73-1.65 (m, 2H), 1.51-1.43 (m, 2H), 1.34 (d, 2H, J=10.4 Hz), 1.32-1.25 (m, 2H). LC-MS: MS (ES⁺): m/z=612.2 [M+H⁺−100].

### 1.3 Preparation of Compound 4

To a solution of tert-butyl-N—[(E)-7-[(3R)-3-[4-amino-3-(4-phenoxyphenyl) pyrazolo[3,4-d]pyrimidin-1-yl]-1-piperidyl]-7-oxo-hept-5-enyl]-N-tert-butoxycarbonyl-carbamate (0.20 g, 0.28 mmol, 1.0 equiv) in DCM (1.5 mL) was added TFA (0.77 g, 6.75 mmol, 0.5 mL, 24.0 equiv). The mixture was stirred at 25° C. for 0.5 h. The mixture was concentrated under reduced pressure to give the crude product (E)-7-amino-1-[(3R)-3-[4-amino-3-(4-phenoxyphenyl) pyrazolo[3,4-d]pyrimidin-1-yl]-1-piperidyl]hept-2-en-1-one (0.17 g, 0.27 mmol, 97% yield, TFA salt) as a yellow oil. LC-MS: MS (ES⁺): m/z=512.2 [M+H⁺].

### 1.4 Preparation of Compound I-1

To a solution of (E)-7-amino-1-[(3R)-3-[4-amino-3-(4-phenoxyphenyl) pyrazolo[3,4-d]pyrimidin-1-yl]-1-piperidyl]hept-2-en-1-one (85 mg, 0.17 mmol, 1.2 equiv) and 5-[4-amino-3-(2-amino-1,3-benzoxazol-5-yl) pyrazolo[3,4-d]pyrimidin-1-yl]pentanoic acid (50 mg, 0.14 mmol, 1.0 equiv) in DMF (1 mL) were added HATU (67 mg, 177 mmol, 1.3 equiv) and DIEA (53 mg, 408 mmol, 71 mL, 3.0 equiv). The mixture was stirred at 25° C. for 1 h. The solution was purified by prep-HPLC (column: Waters Xbridge 150*25 mm*5 μm; mobile phase: [water (10 mM NH₄HCO₃)-ACN]; B %: 27%-57%, 10 min) to give the desired product 5-[4-amino-3-(2-amino-1,3-benzoxazol-5-yl) pyrazolo[3,4-d]pyrimidin-1-yl]-N—[(E)-7-[(3R)-3-[4-amino-3-(4-phenoxyphenyl) pyrazolo[3,4-d]pyrimidin-1-yl]-1-piperidyl]-7-oxo-hept-5-enyl]pentanamide (52 mg, 59 mmol, 44% yield, 98% purity) as a white solid. ¹H NMR (400 MHz, METHANOL-d₄): δ 8.29-8.17 (m, 2H), 7.70-7.60 (m, 2H), 7.54-7.47 (m, 1H), 7.46-7.28 (m, 4H), 7.18-7.05 (m, 5H), 6.77-6.57 (m, 1H), 6.49-6.16 (m, 1H), 4.47-4.30 (m, 2H), 4.26-3.56 (m, 2H), 3.67-3.56 (m, 1H), 3.52-3.33 (m, 1H), 3.26-3.04 (m, 3H), 2.97 (d, 1H, J=10.4 Hz), 2.83-2.27 (m, 2H), 2.26-2.12 (m, 4H), 2.11-1.80 (m, 4H), 1.71-1.24 (m, 8H). LC-MS: MS (ES⁺): RT=2.285 min, m/z=861.4 [M+H⁺], LC-MS METHOD 10.

### Example 2—Synthesis of Compound I-2

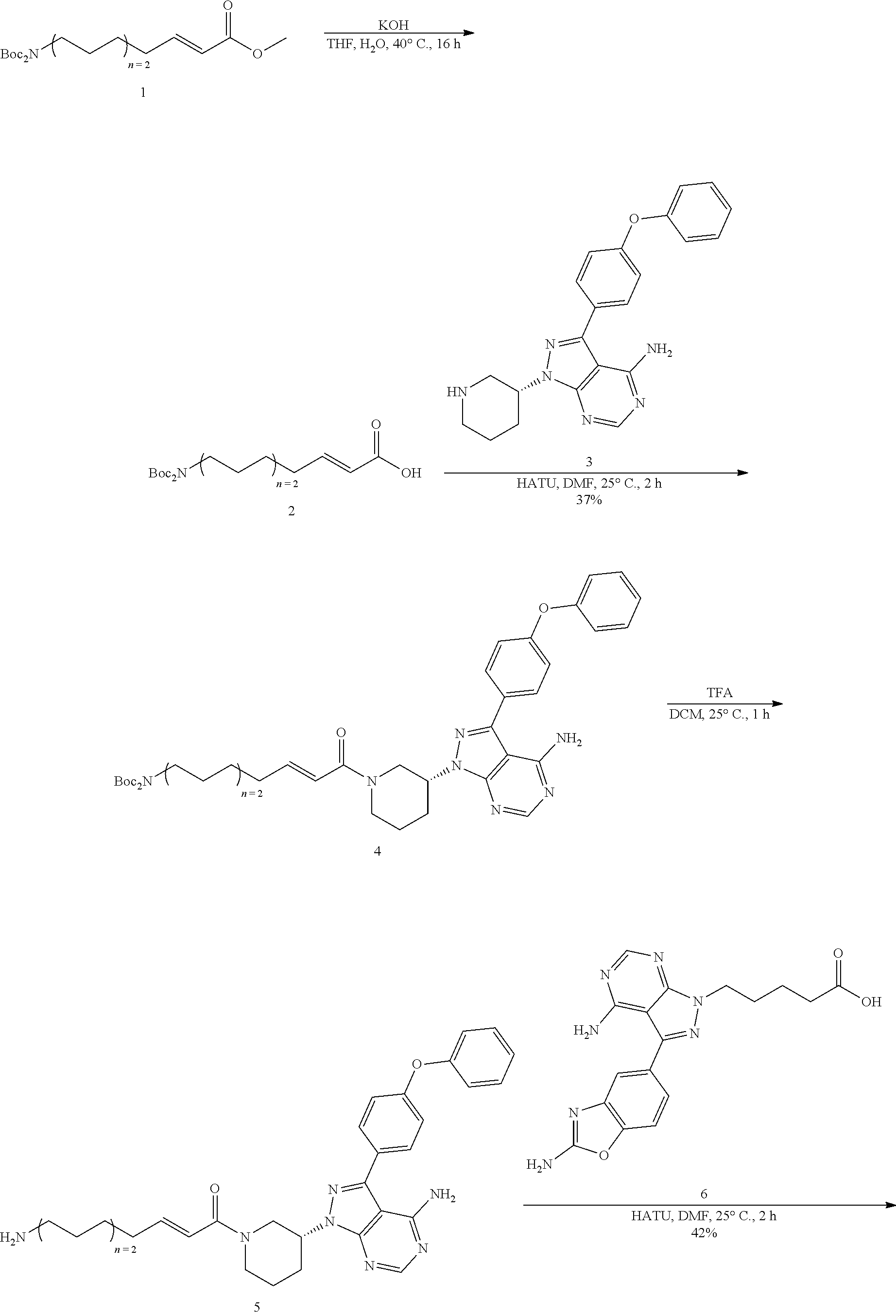

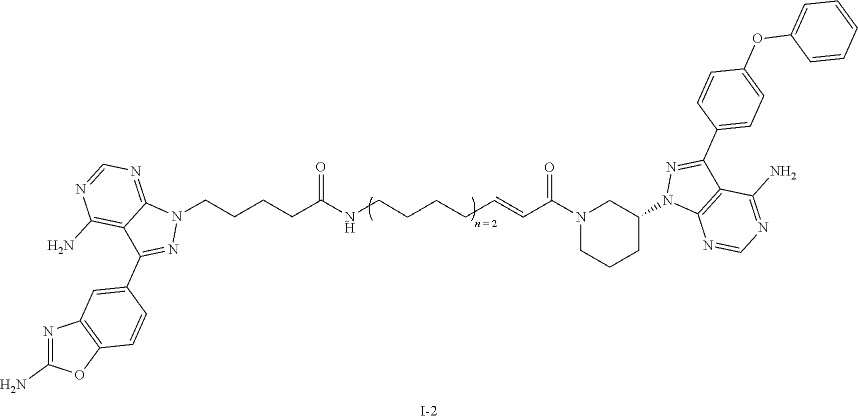

### 2.1 Preparation of Compound 2

To a mixture of methyl (E)-11-[bis(tert-butoxycarbonyl)amino]undec-2-enoate (400 mg, 967 mmol, 1.0 equiv) in THF (3.0 mL) and H₂O (0.5 mL) was added KOH (162 mg, 2.90 mmol, 3.0 equiv). The mixture was stirred at 40° C. for 16 h. The mixture was concentrated to give the crude product (E)-11-[bis(tert-butoxycarbonyl)amino]undec-2-enoic acid (386 mg, crude) as a light yellow solid.

### 2.2 Preparation of Compound 4

To a mixture of (E)-11-[bis(tert-butoxycarbonyl)amino]undec-2-enoic acid (386 mg, 966 mmol, 1.0 equiv) and 3-(4-phenoxyphenyl)-1-[(3R)-3-piperidyl]pyrazolo[3,4-d]pyrimidin-4-amine (373 mg, 965 mmol, 1.0 equiv) in DMF (4.0 mL) were added DIEA (499 mg, 3.86 mmol, 4.0 equiv) and HATU (440 mg, 1.16 mmol, 1.2 equiv). The mixture was stirred at 25° C. for 2 h. The mixture was purified by prep-HPLC (column: Waters X bridge C18 150*50 mm* 10 μm; mobile phase: [water (10 mM NH₄HCO₃)-ACN]; B %: 65%-95%, 10 min) to give tert-butyl-N—[(E)-11-[(3R)-3-[4-amino-3-(4-phenoxyphenyl) pyrazolo[3,4-d]pyrimidin-1-yl]-1-piperidyl]-11-oxo-undec-9-enyl]-N-tert-butoxycarbonyl-carbamate (280 mg, 364 mmol, 37% yield) as a colorless oil. LC-MS: MS (ES⁺): m/z=768.4 [M+H⁺].

### 2.3 Preparation of Compound 5

To a mixture of tert-butyl-N—[(E)-11-[(3R)-3-[4-amino-3-(4-phenoxyphenyl) pyrazolo[3,4-d]pyrimidin-1-yl]-1-piperidyl]-11-oxo-undec-9-enyl]-N-tert-butoxycarbonyl-carbamate (280 mg, 364 mmol, 1.0 equiv) in DCM (3 mL) was added TFA (1.54 g, 13.5 mmol, 1 mL, 37.0 equiv). The mixture was stirred at 25° C. for 1 h. The mixture was concentrated to give the crude product (E)-11-amino-1-[(3R)-3-[4-amino-3-(4-phenoxyphenyl) pyrazolo[3,4-d]pyrimidin-1-yl]-1-piperidyl]undec-2-en-1-one (248 mg, crude, TFA salt) as a yellow gum. LC-MS: MS (ES⁺): m/z=568.2 [M+H⁺].

### 2.4 Preparation of Compound I-2

To a mixture of (E)-11-amino-1-[(3R)-3-[4-amino-3-(4-phenoxyphenyl) pyrazolo[3,4-d]pyrimidin-1-yl]-1-piperidyl]undec-2-en-1-one (124 mg, 182 mmol, 1.0 equiv, TFA salt) and 5-[4-amino-3-(2-amino-1,3-benzoxazol-5-yl)-pyrazolo[3,4-d]pyrimidin-1-yl]pentanoic acid (67 mg, 0.18 mmol, 1.0 equiv) in DMF (2 mL) were added DIEA (94.0 mg, 728 mmol, 126.7 mL, 4.0 equiv) and HATU (83 mg, 0.22 mmol, 1.2 equiv). The mixture was stirred at 25° C. for 2 h. The mixture was purified by prep-HPLC (column: Phenomenex Gemini-NX C18 75*30 mm *3 μm; mobile phase: [water (10 mM NH₄HCO₃)-ACN]; B %: 34%-64%, 8 min) to give 5-[4-amino-3-(2-amino-1,3-benzoxazol-5-yl) pyrazolo[3,4-d]pyrimidin-1-yl]-N—[(E)-11-[(3R)-3-[4-amino-3-(4-phenoxyphenyl) pyrazolo[3,4-d]pyrimidin-1-yl]-1-piperidyl]-11-oxo-undec-9-enyl]pentanamide (70 mg, 76 mmol, 42% yield, 100% purity) as a white solid. ¹H NMR (400 MHZ, MeOD): δ 8.22 (s, 2H), 7.74-7.56 (m, 2H), 7.55-7.46 (m, 1H), 7.46-7.23 (m, 4H), 7.23-7.01 (m, 5H), 6.83-6.55 (m, 1H), 6.51-6.14 (m, 1H), 4.60-3.83 (m, 5H), 3.56-3.34 (m, 1H), 3.18-3.01 (m, 2H), 2.32-1.82 (m, 9H), 1.73-1.56 (m, 3H), 1.48-1.06 (m, 13H). LC-MS: MS (ES⁺): RT=2.158 min, m/z=917.3 [M+H⁺], LC-MS METHOD 25.

### Example 3—Synthesis of Compound I-3 and Additional Compounds (Synthesized According to General Scheme A)

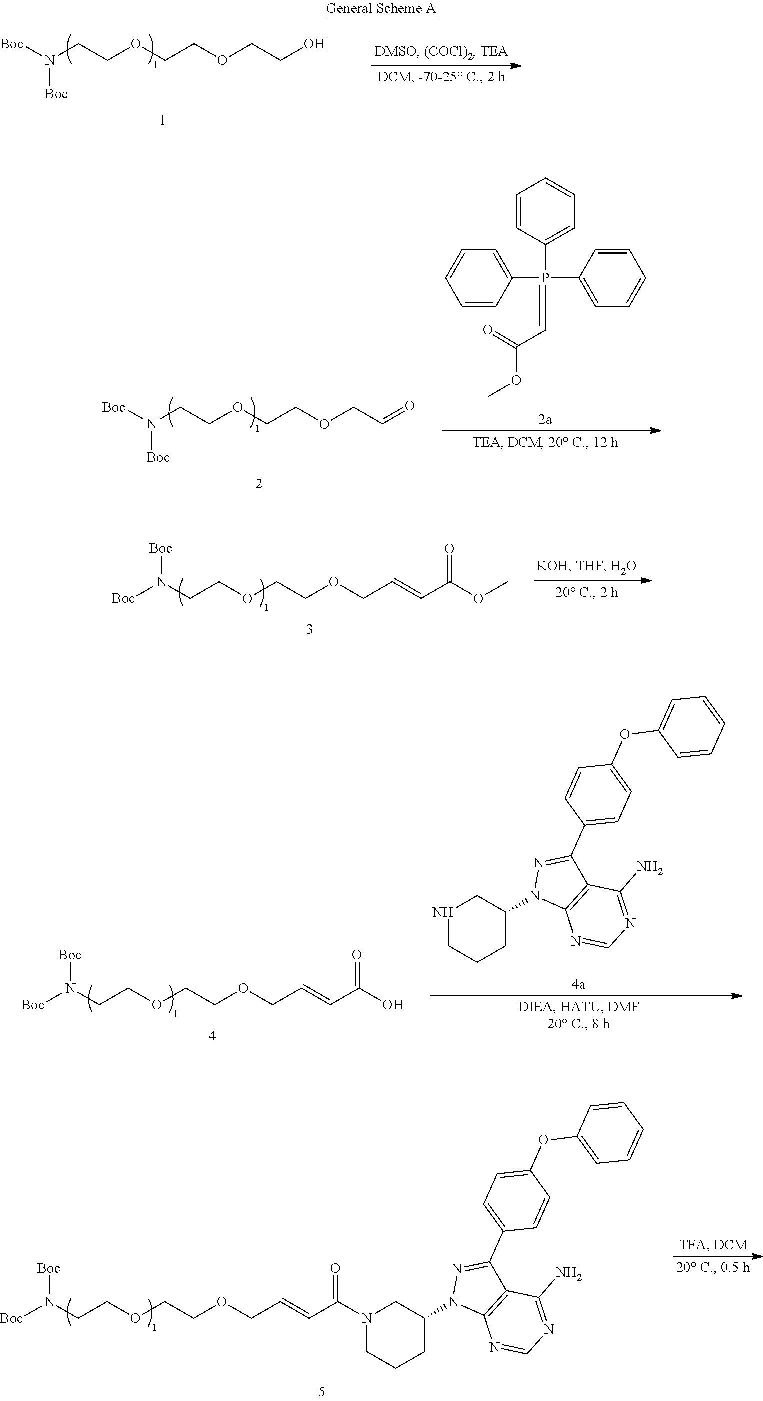

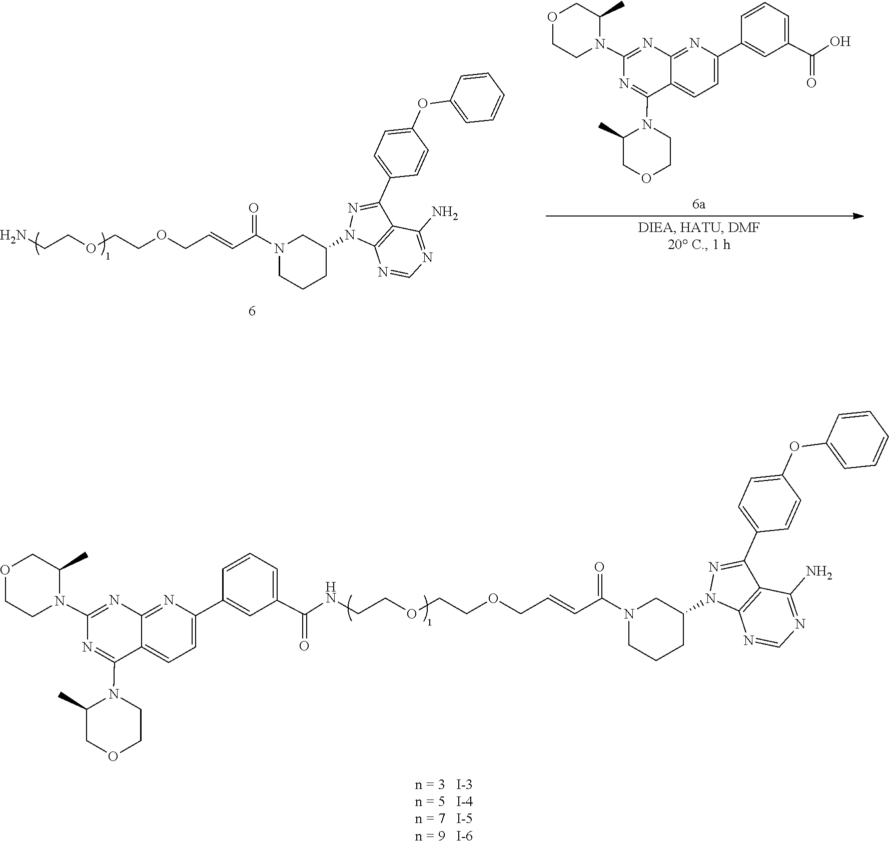

### 3.1 Preparation of Compound 2

To a solution of DMSO (8.93 g, 114 mmol, 10 equiv) in DCM (60 mL) was added (COCl)₂ (7.25 g, 57.1 mmol, 5 equiv) at −70° C. and the mixture was stirred for 0.5 h, then tert-butyl N-tert-butoxycarbonyl-N-[2-[2-[2-[2-(2-hydroxyethoxy)ethoxy]ethoxy]ethoxy]ethyl]carbamate (5 g, 11.4 mmol, 1 equiv) was added. The mixture was stirred at −70° C. for another 0.5 h and then TEA (11.6 g, 114 mmol, 10 equiv) was added dropwise for 0.5 h. The mixture was slowly warmed to 25° C. and stirred for 0.5 h. The mixture was concentrated under reduced pressure to give the desired compound tert-butyl N-tert-butoxycarbonyl-N-[2-[2-[2-[2-(2-oxoethoxy)ethoxy]ethoxy]ethoxy]ethyl]carbamate (4.8 g, crude) as a white solid which was used directly for next step.

### 3.2 Preparation of Compound 3

To a solution of tert-butyl N-tert-butoxycarbonyl-N-[2-[2-[2-[2-(2-oxoethoxy) ethoxy]ethoxy]ethoxy]ethyl]carbamate (4.8 g, 11 mmol, 1 equiv) in DCM (10 mL) was added TEA (3.35 g, 33 mmol, 3 equiv) and methyl 2-(triphenyl-2.5-phosphanylidene) acetate (4.42 g, 13.3 mmol, 1.2 equiv). The mixture was stirred at 20° C. for 12 h. The mixture was concentrated and the residue was purified by prep-HPLC (column: Waters Xbridge BEH C18 250*50 mm*10 μm; mobile phase: [water (0.05% ammonia hydroxide v/v)-ACN]; B %: 40%-70%, 20 min) to give the desired compound methyl (E)-4-[2-[2-[2-[2-[bis(tert-butoxycarbonyl) amino]ethoxy]ethoxy]ethoxy]ethoxy]but-2-enoate (2 g, 36% yield) was obtained as a yellow oil. ¹H NMR (400 MHZ, CDCl₃): δ 7.00-6.94 (m, 1H), 6.19-5.99 (m, 1H), 4.22-4.17 (m, 2H), 3.83-3.59 (m, 19H), 1.58-1.45 (m, 18H).

### 3.3 Preparation of Compound 4

To a solution of methyl (E)-4-[2-[2-[2-[2-[bis(tert-butoxycarbonyl)amino]ethoxy]ethoxy]ethoxy]ethoxy]but-2-enoate (130 mg, 264 mmol, 1 equiv) in THF (2 mL) and H₂O (1 mL) was added KOH (22 mg, 396 mmol, 1.5 equiv). The mixture was stirred at 20° C. for 2 h. The pH value of the mixture was adjusted to about 5 by adding HCl solution (1 M) and then the mixture was concentrated under reduced pressure to give the desired compound (E)-4-[2-[2-[2-[2-[bis(tert-butoxycarbonyl)amino]ethoxy]ethoxy]ethoxy]ethoxy]but-2-enoic acid (125 mg, crude) as a white solid which was used directly for next step. LC-MS: MS (ES⁺): m/z=495.5 [M+H₂O].

### 3.4 Preparation of Compound 5

To a solution of 3-(4-phenoxyphenyl)-1-[(3R)-3-piperidyl]pyrazolo[3,4-d]pyrimidin-4-amine (100 mg, 199 mmol, 1 equiv, TFA salt) and (E)-4-[2-[2-[2-[2-[bis(tert-butoxycarbonyl)amino]ethoxy]ethoxy]ethoxy]ethoxy]but-2-enoic acid (95 mg, 199 mmol, 1 equiv) in DMF (2 mL) was added DIEA (129 mg, 999 mmol, 5 equiv) and HATU (83 mg, 219 mmol, 1.1 equiv). The mixture was stirred at 20° C. for 8 h and concentrated under reduced pressure. The residue was purified by prep-HPLC (column: Waters Xbridge 150*25 mm*5 μm; mobile phase: [water (10 mM NH₄HCO₃)-ACN]; B %: 55%-88%, 8 min) to give tert-buty1N-[2-[2-[2-[2-[(E)-4-[(3R)-3-[4-amino-3-(4-phenoxyphenyl) pyrazolo[3,4-d]pyrimidin-1-yl]-1-piperidyl]-4-oxo-but-2-enoxy]ethoxy]ethoxy]ethoxy]ethyl]-N-tert-butoxycarbonyl-carbamate (120 mg, 70% yield) as a yellow oil. LC-MS: MS (ES⁺): m/z=846.4 [M+H⁺].

### 3.5 Preparation of Compound 6

To a solution of tert-butyl N-[2-[2-[2-[2-[(E)-4-[(3R)-3-[4-amino-3-(4-phenoxyphenyl)-pyrazolo[3,4-d]pyrimidin-1-yl]-1-piperidyl]-4-oxo-but-2-enoxy]ethoxy]ethoxy]ethoxy]ethyl]-N-tert-butoxycarbonyl-carbamate (100 mg, 118 mmol, 1 equiv) in DCM (2 mL) was added TFA (2 mL). The mixture was stirred at 20° C. for 0.5 h. The reaction mixture was concentrated to give the crdue compound (E)-4-[2-[2-[2-(2-aminoethoxy)ethoxy]ethoxy]ethoxy]-1-[(3R)-3-[4-amino-3-(4-phenoxyphenyl) pyrazolo[3,4-d]pyrimidin-1-yl]-1-piperidyl]but-2-en-1-one (80 mg, crude, TFA salt) as a yellow oil. LC-MS: MS (ES⁺): m/z=678.3 [M+Na⁺].

### 3.6 Preparation of Compound I-3

To a solution of (E)-4-[2-[2-[2-(2-aminoethoxy)ethoxy]ethoxy]ethoxy]-1-[(3R)-3-[4-amino-3-(4-phenoxyphenyl) pyrazolo[3,4-d]pyrimidin-1-yl]-1-piperidyl]but-2-en-1-one (80 mg, 105 mmol, 1 equiv, TFA salt) and 3-[2,4-bis[(3R)-3-methylmorpholin-4-yl]pyrido[2,3-d]pyrimidin-7-yl]benzoic acid (47 mg, 105 mmol, 1 equiv) in DMF (2 mL) was added DIEA (68 mg, 526 mmol, 5 equiv) and HATU (44 mg, 115 mmol, 1.1 equiv). The mixture was stirred at 20° C. for 1 h and concentrated. The residue was purified by prep-HPLC (column: Waters Xbridge 150*25 mm*5 μm; mobile phase: [water (10 mM NH₄HCO₃)-ACN]; B %: 45%-75%, 9 min) to give the desired compound N-[2-[2-[2-[2-[(E)-4-[(3R)-3-[4-amino-3-(4-phenoxyphenyl) pyrazolo[3,4-d]pyrimidin-1-yl]-1-piperidyl]-4-oxo-but-2-enoxy]ethoxy]ethoxy]ethoxy]ethyl]-3-[2,4-bis[(3R)-3-methylmorpholin-4-yl]pyrido[2,3-d]pyrimidin-7-yl]benzamide (72 mg, 63% yield) as a white solid. ¹H NMR (400 MHZ, DMSO-d₆): δ 8.85-8.58 (m, 2H), 8.44-8.08 (m, 3H), 7.96 (d, J=7.2 Hz, 1H), 7.76-7.52 (m, 4H), 7.43 (t, J=8.0 Hz, 2H), 7.29-6.85 (m, 5H), 6.72-6.48 (m, 1H), 4.80-4.55 (m, 2H), 4.42 (d, J=14.0 Hz, 2H), 4.26-3.88 (m, 5H), 3.81-3.38 (m, 25H), 3.24-3.12 (m, 2H), 2.30-2.02 (m, 2H), 1.89 (s, 1H), 1.56 (s, 1H), 1.43-1.11 (m, 6H), 0.94 (d, J=6.4 Hz, 1H). LC-MS: MS (ES⁺): RT=2.509 min, m/z=1077.5 [M+H⁺], LC-MS METHOD 10.

### Example 4—Synthesis of Compound I-4 (Synthesized According to General Scheme a in a Similar Manner to Compound I-3

N-[2-[2-[2-[2-[2-[2-[(E)-4-[(3R)-3-[4-amino-3-(4-phenoxyphenyl) pyrazolo[3,4-d]pyrimidin-1-yl]-1-piperidyl]-4-oxo-but-2-enoxy]ethoxy]ethoxy]ethoxy]ethoxy]ethoxy]ethyl]-3-[2,4-bis[(3R)-3-methylmorpholin-4-yl]pyrido[2,3-d]pyrimidin-7-yl]benzamide (70 mg, 54% yield) as a yellow solid. ¹H NMR (400 MHZ, DMSO-d₆): δ 8.72 (s, 1H), 8.65 (s, 1H), 8.34-8.20 (m, 3H), 7.97 (d, 1H, J=7.6 Hz), 7.72-7.60 (m, 4H), 7.44 (t, 2H, J=8.0 Hz), 7.21-7.11 (m, 5H), 6.71-6.46 (m, 2H), 4.76 (s, 2H), 4.43 (d, 2H, J=12.4 Hz), 4.15 (s, 2H), 4.01 (s, 1H), 3.96-3.87 (m, 3H), 3.77-3.72 (m, 2H), 3.68-3.44 (m, 27H), 3.30 (s, 8H), 2.24 (s, 1H), 2.13 (s, 1H), 1.92 (s, 1H), 1.57 (s, 1H), 1.38 (d, 3H, J=6.8 Hz), 1.25 (d, 3H, J=6.8 Hz). LC-MS: MS (ES⁺): RT=2.526 min, m/z=1165.6 [M+H⁺], LC-MS METHOD 10.

### Example 5—Synthesis of Compound I-5 (Synthesized According to General Scheme a in a Similar Manner to Compound I-3)

N-[2-[2-[2-[2-[2-[2-[2-[2-[(E)-4-[(3R)-3-[4-amino-3-(4-phenoxyphenyl) pyrazolo[3,4-d]pyrimidin-1-yl]-1-piperidyl]-4-oxo-but-2-enoxy]ethoxy]ethoxy]ethoxy]ethoxy]ethoxy]ethoxy]ethoxy]ethyl]-3-[2,4-bis[(3R)-3-methylmorpholin-4-yl]pyrido[2,3-d]pyrimidin-7-yl]benzamide (34 mg, 24% yield) as a yellow solid. ¹H NMR (400 MHZ, CD₃OD): δ 8.6 (s, 1H), 8.31-8.24 (m, 3H), 7.95 (d, 1H, J=7.6 Hz), 7.68-7.58 (m, 4H), 7.42-7.38 (m, 2H), 7.19-7.07 (m, 5H), 6.74-6.52 (m, 3H), 4.59-4.48 (m, 5H), 4.19-3.50 (m, 47H), 2.37-2.06 (m, 3H), 1.71-1.66 (m, 1H), 1.48-1.45 (m, 3H), 1.36-1.33 (m, 3H). LC-MS: MS (ES⁺): RT=2.17 min, m/z=1154.6 [M+H⁺], LC-MS METHOD 25.

### Example 6—Synthesis of Compound I-6 (Synthesized According to General Scheme a in a Similar Manner to Compound I-3)

N-[2-[2-[2-[2-[2-[2-[2-[2-[2-[2-[(E)-4-[(3R)-3-[4-amino-3-(4-phenoxyphenyl) pyrazolo[3,4-d]pyrimidin-1-yl]-1-piperidyl]-4-oxo-but-2-enoxy]ethoxy]ethoxy]ethoxy]ethoxy]ethoxy]ethoxy]ethoxy]ethoxy]ethoxy]ethyl]-3-[2,4-bis[(3R)-3-methylmorpholin-4-yl]pyrido[2,3-d]pyrimidin-7-yl]benzamide (78.18 mg, 38% yield) as a yellow solid. ¹H NMR (400 MHZ, MeOD): δ 8.67 (s, 1H), 8.42-8.21 (m, 3H), 8.00 (d, J=8.0 Hz, 1H), 7.85 (d, J=8.0 Hz, 1H), 7.75-7.62 (m, 3H), 7.50-7.37 (m, 2H), 7.28-6.96 (m, 5H), 6.83-6.45 (m, 2H), 4.83-4.37 (m, 4H), 4.26-3.58 (m, 40H), 3.53-3.28 (m, 17H), 2.43-2.05 (m, 3H), 1.81-1.63 (m, 1H), 1.57 (d, J=6.8 Hz, 3H), 1.42 (d, J=6.8 Hz, 3H). LC-MS: MS (ES⁺): RT=2.159 min, m/z=1341.6 [M+H⁺], LC-MS METHOD 25.

### Example 7—Synthesis of Compound I-7

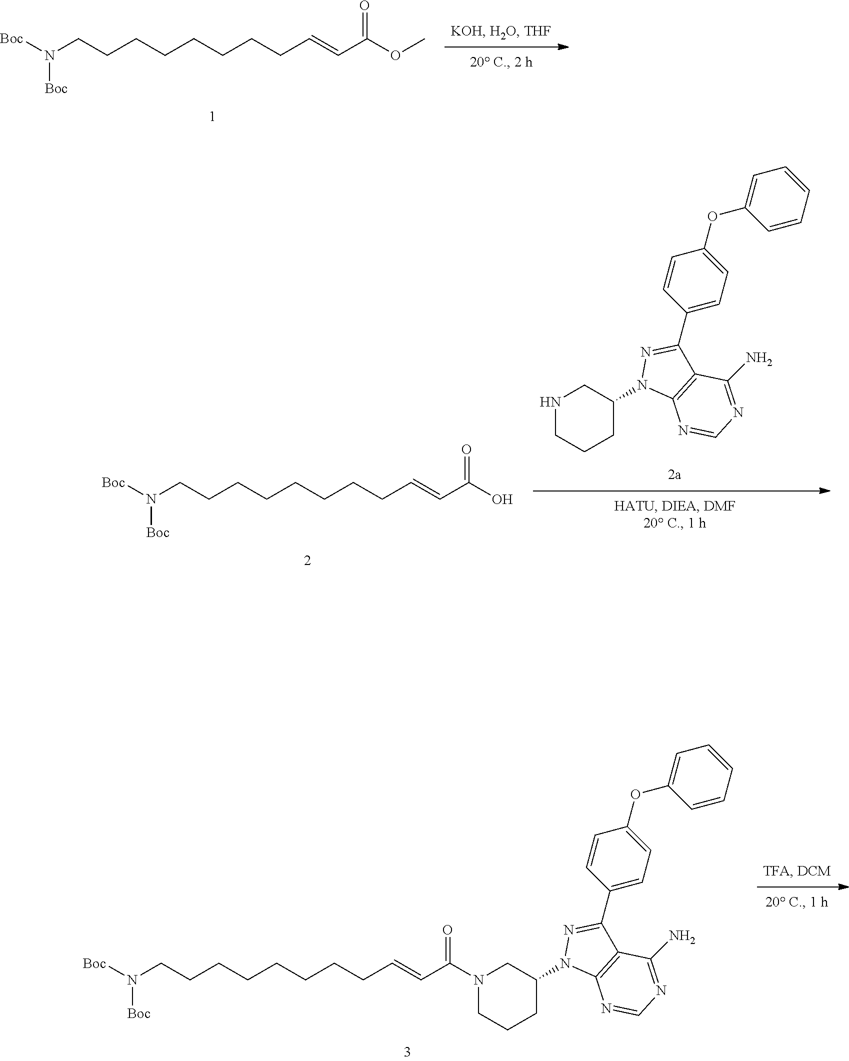 [SMILES: COC(=O)/C=C/CCCCCCCCN(C(=O)OC(C)(C)C)C(=O)OC(C)(C)C ; CC(C)(C)OC(=O)N(CCCCCCCC/C=C/C(=O)O)C(=O)OC(C)(C)C ; Nc1ncnc2c1c(-c1ccc(Oc3ccccc3)cc1)nn2[C@@H]1CCCNC1 ; CC(C)(C)OC(=O)N(CCCCCCCC/C=C/C(=O)N1CCC[C@@H](n2nc(-c3ccc(Oc4ccccc4)cc3)c3c(N)ncnc32)C1)C(=O)OC(C)(C)C]

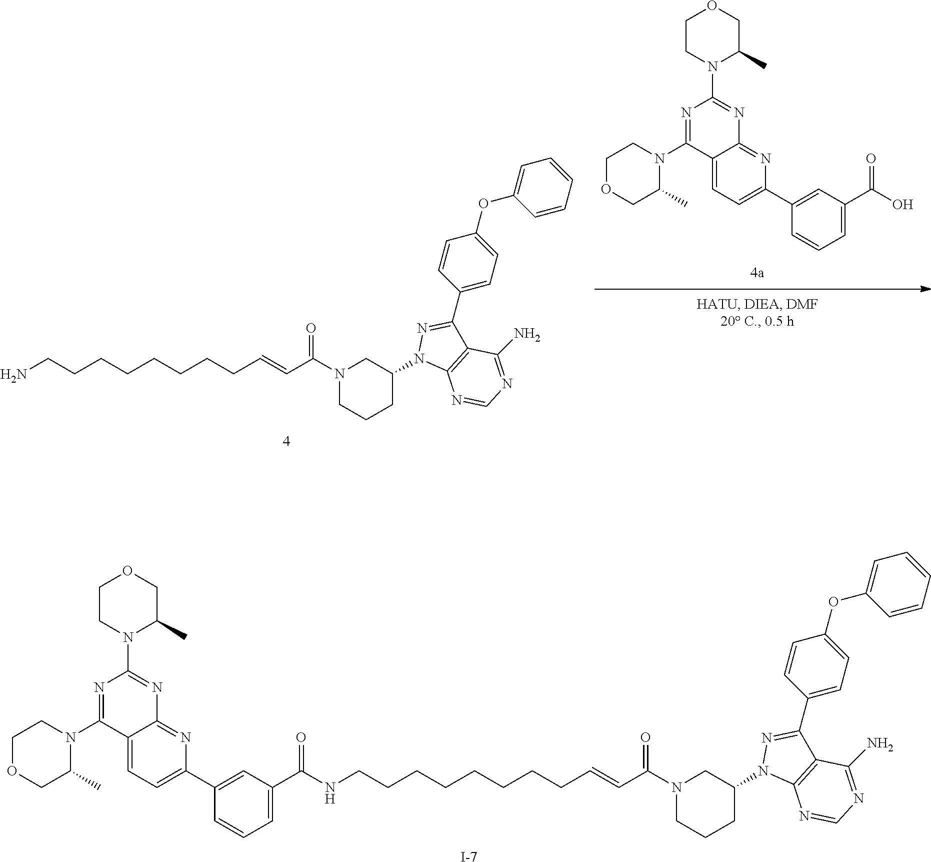

### 7.1 Preparation of Compound 2

To a solution of methyl (E)-11-[bis(tert-butoxycarbonyl)amino]undec-2-enoate (200 mg, 483 mmol, 1 equiv) in H₂O (1 mL) and THF (2 mL) was added KOH (40 mg, 725 mmol, 1.5 equiv). The mixture was stirred at 20° C. for 2 h and then the pH value of the mixture was adjusted to about 5 by adding HCl solution (1 M). The mixture was concentrated to give (E)-11-[bis(tert-butoxycarbonyl)amino]undec-2-enoic acid (190 mg, crude) as a white solid which was used directly for next step.

### 7.2 Preparation of Compound 3

To a solution of (E)-11-[bis(tert-butoxycarbonyl)amino]undec-2-enoic acid (190 mg, 475 mmol, 1 equiv) and 3-(4-phenoxyphenyl)-1-[(3R)-3-piperidyl]pyrazolo[3,4-d]pyrimidin-4-amine (238 mg, 475 mmol, 1 equiv, TFA salt) in DMF (3 mL) was added DIEA (184 mg, 1.43 mmol, 3 equiv) and HATU (216 mg, 570 mmol, 1.2 equiv). The mixture was stirred at 20° C. for 1 h. The mixture was concentrated and the residue was purified by prep-HPLC (column: Phenomenex Synergi C18 150*25 mm*10 μm; mobile phase: [water (0.1% TFA)-ACN]; B %: 65%-95%, 10 min) to give tert-butyl N—[(E)-11-[(3R)-3-[4-amino-3-(4-phenoxyphenyl) pyrazolo[3,4-d]pyrimidin-1-yl]-1-piperidyl]-11-oxo-undec-9-enyl]-N-tert-butoxycarbonyl-carbamate (80 mg, 21% yield) as a yellow oil. LC-MS: MS (ES⁺): m/z=768.5 [M+H⁺].

### 7.3 Preparation of Compound 4

To a solution of tert-butyl N—[(E)-11-[(3R)-3-[4-amino-3-(4-phenoxyphenyl) pyrazolo[3,4-d]pyrimidin-1-yl]-1-piperidyl]-11-oxo-undec-9-enyl]-N-tert-butoxycarbonyl-carbamate (70 mg, 91 mmol, 1 equiv) in DCM (2 mL) was added TFA (3.08 g, 27.01 mmol, 296.3 equiv). The mixture was stirred at 20° C. for 1 h and then concentrated to give (E)-11-amino-1-[(3R)-3-[4-amino-3-(4-phenoxyphenyl)-pyrazolo[3,4-d]pyrimidin-1-y]]-1-piperidyl]undec-2-en-1-one (62 mg, crude, TFA salt) as a yellow oil which was used directly for next step. LC-MS: MS (ES⁺): m/z=568.4 [M+H⁺].

### 7.4 Preparation of Compound I-7

To a solution of (E)-11-amino-1-[(3R)-3-[4-amino-3-(4-phenoxyphenyl) pyrazolo[3,4-d]pyrimidin-1-yl]-1-piperidyl]undec-2-en-1-one (60 mg, 88 mmol, 1 equiv, TFA salt) and 3-[2,4-bis[(3R)-3-methylmorpholin-4-yl]pyrido[2,3-d]pyrimidin-7-yl]benzoic acid (39 mg, 88 mmol, 1 equiv) in DMF (2 mL) was added DIEA (56 mg, 440 mmol, 5 equiv) and HATU (36 mg, 96 mmol, 1.1 equiv). The mixture was stirred at 20° C. for 0.5 h and then concentrated. The residue was purified by prep-HPLC (column: Phenomenex Gemini-NX C18 75*30 mm*3 μm; mobile phase: [water (10 mM NH₄HCO₃)-ACN]; B %: 56%-86%, 8 min) to give N—[(E)-11-[(3R)-3-[4-amino-3-(4-phenoxyphenyl) pyrazolo[3,4-d]pyrimidin-1-yl]-1-piperidyl]-11-oxo-undec-9-enyl]-3-[2,4-bis[(3R)-3-methylmorpholin-4-yl]pyrido[2,3-d]pyrimidin-7-yl]benzamide (17 mg, 19% yield) as a yellow solid. ¹H NMR (400 MHZ, DMSO-d₆): δ 8.63 (s, 2H), 8.41-8.15 (m, 3H), 7.95 (d, J=8.0 Hz, 1H), 7.79-7.55 (m, 4H), 7.45-7.41 (m, 2H), 7.24-7.04 (m, 5H), 6.70-6.28 (m, 2H), 4.80-4.42 (m, 4H), 4.09 (s, 1H), 4.00-3.41 (m, 11H), 3.30-3.05 (m, 4H), 2.30-1.91 (m, 5H), 1.67-1.16 (m, 20H). LC-MS: MS (ES⁺): RT=2.805 min, m/z=999.5 [M+H⁺], LC-MS METHOD 10.

### Example 8—Synthesis of Compound I-8

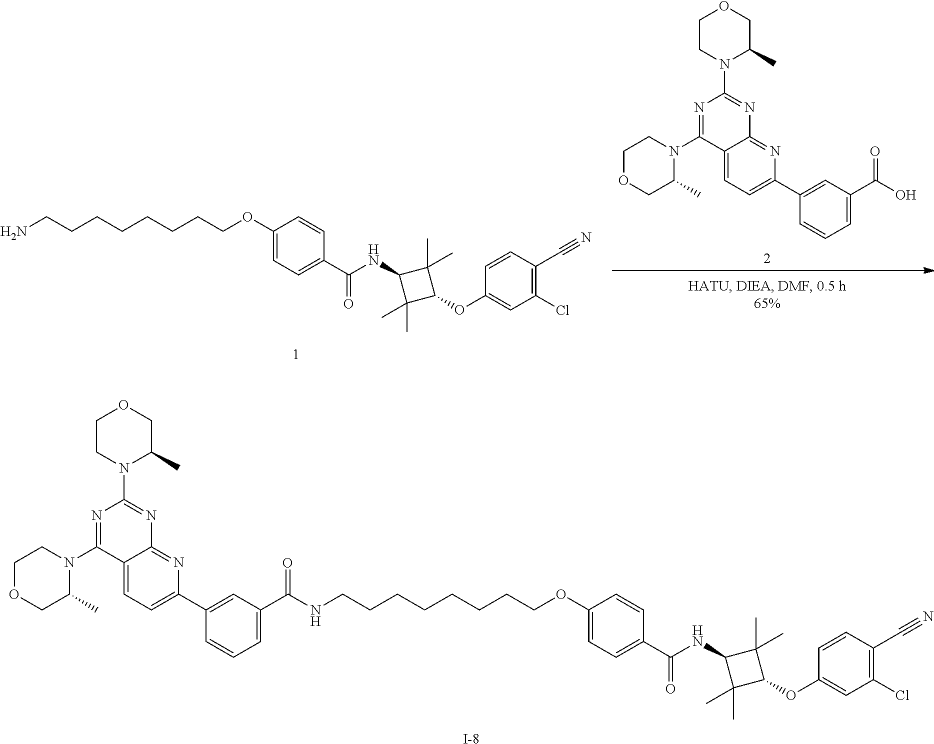

To a solution of 4-(8-aminooctoxy)-N-[3-(3-chloro-4-cyano-phenoxy)-2,2,4,4-tetramethyl-cyclobutyl]benzamide (71 mg, 111 mmol, 1.0 equiv, TFA salt) and DIEA (43 mg, 333 mmol, 3.0 equiv) in DMF (2 mL) was added HATU (50 mg, 133 mmol, 1.2 equiv) and 3-[2,4-bis[(3R)-3-methylmorpholin-4-yl]pyrido[2,3-d]pyrimidin-7-yl]benzoic acid (50 mg, 111 mmol, 1 equiv). The mixture was stirred at 20° C. for 0.5 h and purified by prep-HPLC (column: Waters Xbridge 150*25 mm*5 μm; mobile phase: [water (10 mM NH₄HCO₃)-ACN]; B %: 68%-98%,9 min) to give 3-[2,4-bis[(3R)-3-methylmorpholin-4-yl]pyrido[2,3-d]pyrimidin-7-yl]-N-[8-[4-[[3-(3-chloro-4-cyano-phenoxy)-2,2,4,4-tetramethyl-cyclobutyl]carbamoyl]phenoxy]octyl]benzamide (70 mg, 65% yield). ¹H NMR (400 MHZ, DMSO-d₆): δ 8.66-8.63 (2H, m), 8.26-8.23 (2H, m), 7.92-7.89 (2H, m), 7.83 (d, J=8.8 Hz, 2H), 7.73-7.71 (m, 1H), 7.70-7.63 (m, 2H), 7.21 (d, J=2.4 Hz, 1H), 7.03-6.99 (m, 3H), 4.77 (s, 1H), 4.44 (d, J=13.6 Hz, 2H), 4.32 (s, 1H), 4.13-4.07 (m, 3H), 3.96-3.88 (m, 3H), 3.73-3.68 (m, 2H), 3.64-3.62 (m, 4H), 3.25-3.22 (m, 3H), 1.73-1.71 (m, 2H), 1.57 (s, 2H), 1.43-1.38 (m, 12H), 1.35-1.22 (m, 9H), 1.13 (s, 6H). LC-MS: MS (ES⁺): RT=3.110 min, m/z=957.4 [M+H⁺], LC-MS METHOD 10.

### Example 9—Synthesis of Compound I-9 (Synthesized According to General Scheme B)

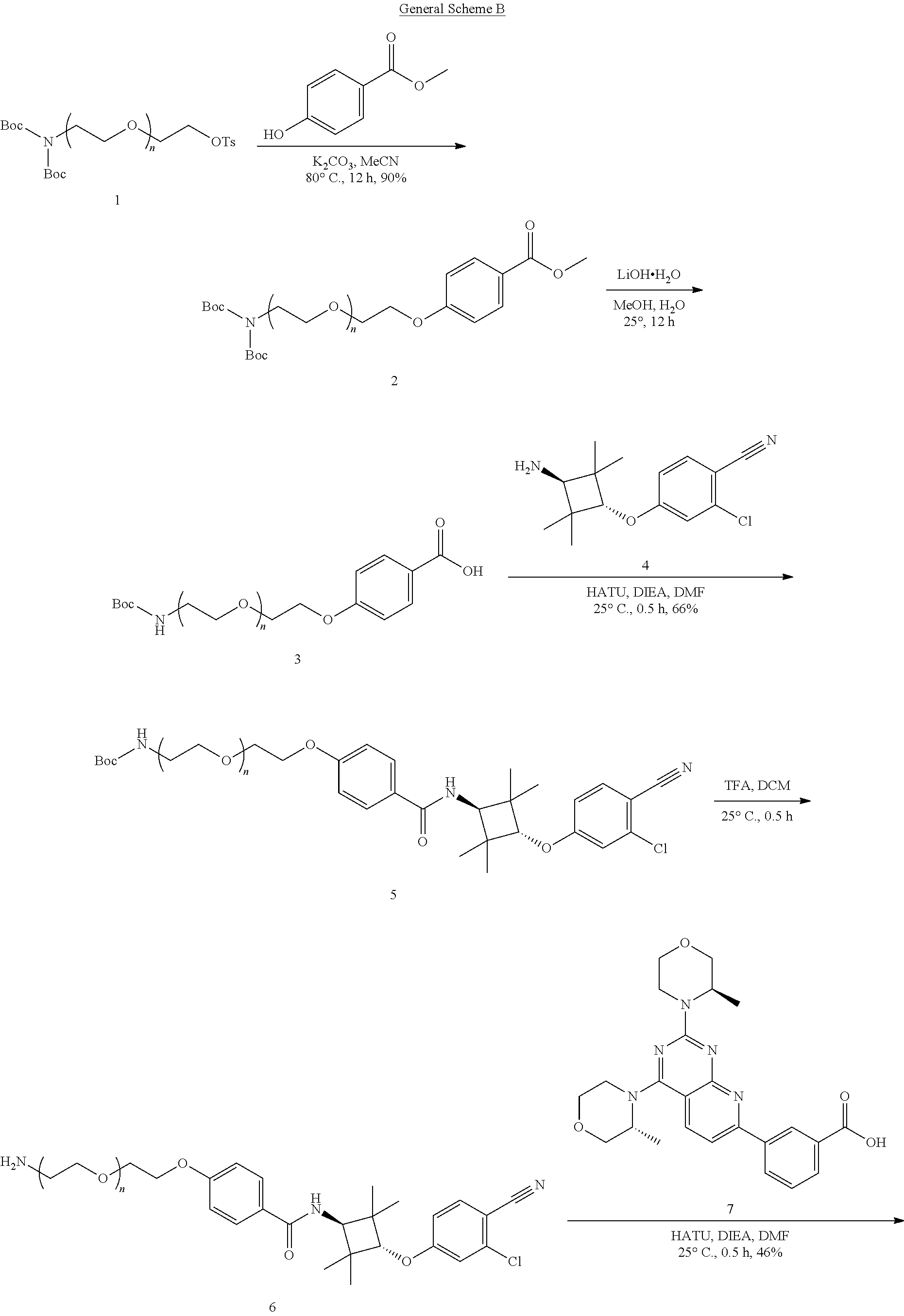

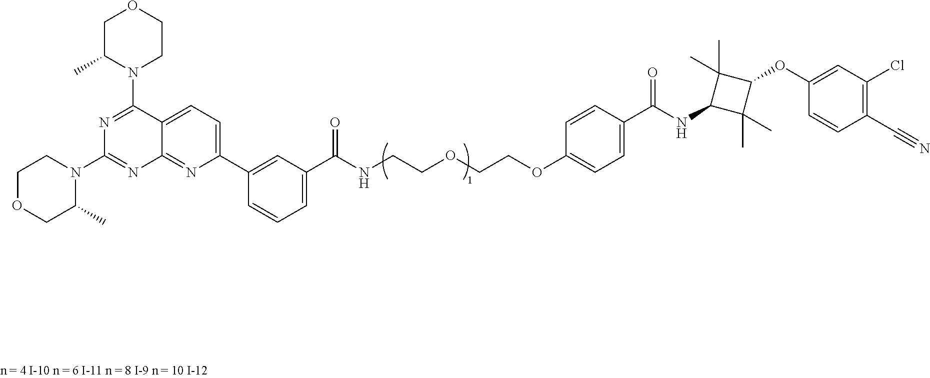

### 9.1 Preparation of Compound 2

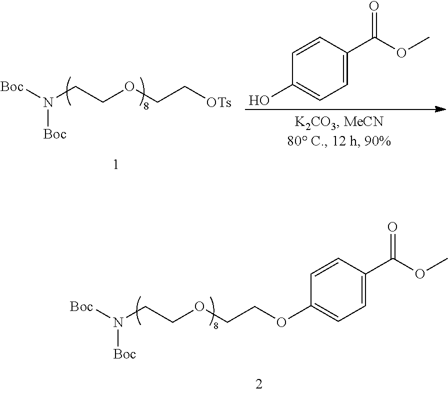

To a solution of 2-[2-[2-[2-[2-[2-[2-[2-[2-[bis(tert-butoxycarbonyl)amino]ethoxy]ethoxy]ethoxy]ethoxy]-ethoxy]ethoxy]ethoxy]ethoxy]ethyl 4-methylbenzenesulfonate (400 mg, 521 mmol, 1 equiv) in MeCN(10 mL) was added K₂CO₃ (216 mg, 1.6 mmol, 3 equiv) and methyl 4-hydroxybenzoate (95 mg, 625 mmol, 1.2 equiv). The mixture was stirred at 80° C. for 12 h. The mixture was concentrated and the residue was purified by column chromatography (SiO₂, Petroleum ether/Ethyl acetate=5/1 to 1/1) to give methyl 4-[2-[2-[2-[2-[2-[2-[2-[2-[2-[bis(tert-butoxycarbonyl)amino]ethoxy]ethoxy]ethoxy]ethoxy]ethoxy]ethoxy]ethoxy]ethoxy]ethoxy]benzoate (350 mg, 90% yield) as a colorless oil. ¹H NMR (400 MHZ, CDCl₃): δ 7.98 (d, 2H, J=8.8 Hz), 6.94 (d, 2H, J=9.2 Hz), 3.89-3.61 (m, 39H), 1.50 (s, 18H). LC-MS: MS (ES⁺): RT=0.935 min, m/z=765.2 [M+H₂O].

### 9.2 Preparation of Compound 3

To a solution of methyl 4-[2-[2-[2-[2-[2-[2-[2-[2-[2-[bis(tert-butoxycarbonyl)amino]ethoxy]ethoxy]ethoxy]ethoxy]ethoxy]ethoxy]ethoxy]ethoxy]ethoxy]benzoate (350 mg, 468 mmol, 1 equiv) in MeOH (3 mL) and H₂O (1 mL) was added LiOH·H₂O (78 mg, 1.8 mmol, 4 equiv). The mixture was stirred at 25° C. for 12 h and concentrated to give 4-[2-[2-[2-[2-[2-[2-[2-[2-[2-(tert-butoxycarbonylamino)ethoxy]ethoxy]ethoxy]ethoxy]ethoxy]ethoxy]ethoxy]ethoxy]ethoxy]benzoic acid (350 mg, crude) as a white solid which was used directly for next step.

LC-MS: MS (ES⁺): RT=0.865 min, m/z=634.6 [M+H⁺].

### 9.3 Preparation of Compound 5

A mixture of 4-(3-amino-2,2,4,4-tetramethyl-cyclobutoxy)-2-chloro-benzonitrile (100 mg, 255 mmol, 1 equiv, TFA salt), 4-[2-[2-[2-[2-[2-[2-[2-[2-[2-(tert-butoxycarbonylamino)ethoxy]ethoxy]ethoxy]ethoxy]-ethoxy]ethoxy]ethoxy]ethoxy]ethoxy]benzoic acid (161 mg, 255 mmol, 1 equiv), DIEA (98 mg, 764 mmol, 3 equiv) and HATU (106 mg, 280 mmol, 1.1 equiv) in DMF (3 mL) was stirred at 25° C. for 0.5 h. The reaction mixture was concentrated and the residue was purified by prep-HPLC (column: Phenomenex Gemini-NX C18 75*30 mm*3 μm; mobile phase: [water (10 mM NH₄HCO₃)-ACN]; B %: 45%-75%, 8 min) to give tert-butyl N-[2-[2-[2-[2-[2-[2-[2-[2-[2-[4-[[3-(3-chloro-4-cyano-phenoxy)-2,2,4,4-tetramethylcyclobutyl]carbamoyl]phenoxy]ethoxy]ethoxy]ethoxy]ethoxy]ethoxy]ethoxy]ethoxy]ethoxy]ethyl]carbam ate (150 mg, 66% yield) as a colorless oil. LC-MS: MS (ES⁺): RT=1.06 min, m/z=894.7 [M+H⁺].

### 9.4 Preparation of Compound 6

A mixture of tert-butyl N-[2-[2-[2-[2-[2-[2-[2-[2-[2-[4-[3-(3-chloro-4-cyano-phenoxy)-2,2,4,4-tetramethyl-cyclobutyl]carbamoyl]phenoxy]ethoxy]ethoxy]ethoxy]ethoxy]ethoxy]ethoxy]ethoxy]ethoxy]ethyl]carbamate (120 mg, 134 mmol, 1 equiv) in DCM (3 mL) and TFA (1 mL) was stirred at 25° C. for 0.5 h and concentrated to give crude 4-[2-[2-[2-[2-[2-[2-[2-[2-(2-aminoethoxy)ethoxy]ethoxy]ethoxy]-ethoxy]ethoxy]ethoxy]ethoxy]ethoxy]-N-[3-(3-chloro-4-cyano-phenoxy)-2,2,4,4-tetramethyl-cyclobutyl]benzamide (130 mg, TFA salt) as a colorless oil. LC-MS: MS (ES⁺): RT=0.955 min, m/z=794.5 [M+H⁺].

### 9.5 Preparation of Compound I-9

To a solution of 3-[2-[(3R)-3-methylmorpholin-4-yl]-4-(3-methylmorpholin-4-yl) pyrido[2,3-d]pyrimidin-7-yl]benzoic acid (50 mg, 111 mmol, 1 equiv) and 4-[2-[2-[2-[2-[2-[2-[2-[2-(2-aminoethoxy)ethoxy]-ethoxy]ethoxy]ethoxy]ethoxy]ethoxy]ethoxy]ethoxy]-N-[3-(3-chloro-4-cyano-phenoxy)-2,2,4,4-tetramethyl-cyclobutyl]benzamide (101 mg, 127 mmol, 1.1 equiv) in DMF (2 mL) was added DIEA (43 mg, 334 mmol, 3 equiv) and HATU (46 mg, 122 mmol, 1.1 equiv). The mixture was stirred at 25° C. for 0.5 h. The mixture was concentrated and the residue was purified by prep-HPLC (column: Phenomenex Gemini-NX C18 75*30 mm*3 μm; mobile phase: [water (10 mM NH₄HCO₃)-ACN]; B %: 46%-76%,8 min) to give N-[2-[2-[2-[2-[2-[2-[2-[2-[2-[4-[[3-(3-chloro-4-cyano-phenoxy)-2,2,4,4-tetramethyl-cyclobutyl]carbamoyl]phenoxy]ethoxy]ethoxy]ethoxy]ethoxy]ethoxy]ethoxy]ethoxy]ethoxy]ethyl]-3-[2-[(3R)-3-methylmorpholin-4-yl]-4-(3-methylmorpholin-4-yl) pyrido[2,3-d]pyrimidin-7-yl]benzamide (63 mg, 46% yield) as a yellow solid. ¹H NMR (400 MHZ, CD₃OD): δ 8.64-8.62 (m, 1H), 8.33-8.27 (m, 2H), 7.98-7.96 (m, 1H), 7.81-7.61 (m, 2H), 7.61-7.55 (m, 3H), 7.14-7.12 (m, 1H), 7.02-6.94 (m, 3H), 4.55-4.50 (m, 2H), 4.27 (s, 1H), 4.17-4.13 (m, 3H), 4.01-3.96 (m, 3H), 3.84-3.54 (m, 43H), 1.50-1.47 (m, 3H), 1.35-1.34 (m, 3H), 1.27 (s, 6H), 1.21 (s, 6H). LC-MS: MS (ES⁺): RT=2.56 min, m/z=1225.6 [M+H⁺], LC-MS METHOD 25.

### Example 10—Synthesis of Compound I-10 (Synthesized According to General Scheme B in a Similar Manner to Compound I-9)

3-[2,4-bis[(3R)-3-methylmorpholin-4-yl]pyrido[2,3-d]pyrimidin-7-yl]-N-[2-[2-[2-]2-[2-[4-[[3-(3-chloro-4-cyano-phenoxy)-2,2,4,4-tetramethyl-cyclobutyl]carbamoyl]phenoxy]ethoxy]ethoxy]ethoxy]ethoxy]ethyl]benzamide (54 mg, 46% yield) as a yellow solid. ¹H NMR (400 MHZ, DMSO-d₆): δ 8.73 (s, 1H), 8.66 (s, 1H), 8.32 (d, 1H, J=7.2 Hz), 8.24 (d, 1H, J=8.0 Hz), 7.98 (d, 1H, J=7.6 Hz), 7.91 (d, 1H, J=8.8 Hz), 7.83 (d, 2H, J=8.4 Hz), 7.72 (d, 1H, J=8.8 Hz), 7.68-7.61 (m, 2H), 7.21 (s, 1H), 7.01 (d, 3H, J=8.8 Hz), 4.78 (s, 1H), 4.44 (d, 2H, J=12.8 Hz), 4.31 (s, 1H), 4.14 (s, 2H), 4.07 (d, 1H, J=8.8 Hz), 3.97-3.87 (m, 3H), 3.73 (s, 4H), 3.59-3.48 (m, 18H), 3.31 (s, 3H), 3.22 (s, 1H), 1.39 (d, 3H, J=6.4 Hz), 1.26-21.22 (m, 9H), 1.13 (s, 6H). LC-MS: MS (ES⁺): RT=1.973 min, m/z=1049.3 [M+H⁺], LC-MS METHOD 40.

### Example 11—Synthesis of Compound I-11 (Synthesized According to General Scheme B in a Similar Manner to Compound I-9

3-[2,4-bis[(3R)-3-methylmorpholin-4-yl]pyrido[2,3-d]pyrimidin-7-y]]—N-]2-[12-[2-[2-[2-[2-[2-[4-[[3-(3-chloro-4-cyanophenoxy)-2,2,4,4-tetramethyl-cyclobutyl]carbamoyl]phenoxy]ethoxy]ethoxy]ethoxy]ethoxy]ethoxy]ethoxy]ethyl]benzamide (68 mg, 54% yield) as a yellow solid. ¹H NMR (400 MHZ, DMSO-d₆): δ 8.75 (t, 1H, J=5.2 Hz), 8.66 (s, 1H), 8.32 (d, 1H, J=8.0 Hz), 8.24 (d, 1H, J=8.4 Hz), 7.98 (d, 1H, J=7.6 Hz), 7.91 (d, 1H, J=8.8 Hz), 7.83 (d, 2H, J=8.8 Hz), 7.73-7.61 (m, 3H), 7.21 (d, 1H, J=2.4 Hz), 7.03-6.99 (m, 3H), 4.77 (s, 1H), 4.44 (d, 2H, J=13.6 Hz), 4.32 (s, 1H), 4.17-4.13 (m, 2H), 4.07 (d, 1H, J=9.2 Hz), 3.96-3.88 (m, 3H), 3.77-3.73 (m, 4H), 3.68-3.62 (m, 3H), 3.60-3.47 (m, 26H), 3.26-3.15 (m, 1H), 1.39 (d, 3H, J=6.8 Hz), 1.26-1.22 (m, 9H), 1.13 (s, 6H). LC-MS: MS (ES⁺): RT=2.563 min, m/z=1137.4 [M+H⁺], LC-MS METHOD 25.

### Example 12—Synthesis of Compound I-12 (synthesized according to General Scheme B in a similar manner to Compound I-9)

3-[2,4-bis[(3R)-3-methylmorpholin-4-yl]pyrido[2,3-d]pyrimidin-7-yl]-N-[2-[2-[2-[2-[2-[2-[2-[2-[2-[2-[2-[4-[[3-(3-chloro-4-cyano-phenoxy)-2,2,4,4-tetramethyl-cyclobutyl]carbamoyl]phenoxy]ethoxy]ethoxy]ethoxy]ethoxy]ethoxy]ethoxy]ethoxy]ethoxy]ethoxy]ethox y]ethyl]benzamide (48 mg, 35% yield) as a yellow oil. ¹H NMR (400 MHZ, MeOD): 8.63 (s, 1H), 8.38-8.20 (m, 2H), 8.01-7.93 (m, 1H), 7.84-7.76 (m, 2H), 7.74-7.58 (m, 3H), 7.11 (s, 1H), 7.05-6.93 (m, 3H), 4.59-4.46 (m, 2H), 4.33-4.11 (m, 4H), 4.06-3.92 (m, 3H), 3.90-3.50 (m, 51H), 3.33 (s, 1H), 1.54-1.43 (m, 3H), 1.39-1.31 (m, 3H), 1.27 (s, 6H), 1.21 (s, 6H). LC-MS: MS (ES⁺): RT=2.547 min, m/z=1314.5 [M+H⁺], LC-MS METHOD 25.

### Example 13—Synthesis of Compound I-13 (Synthesized According to General Scheme C)

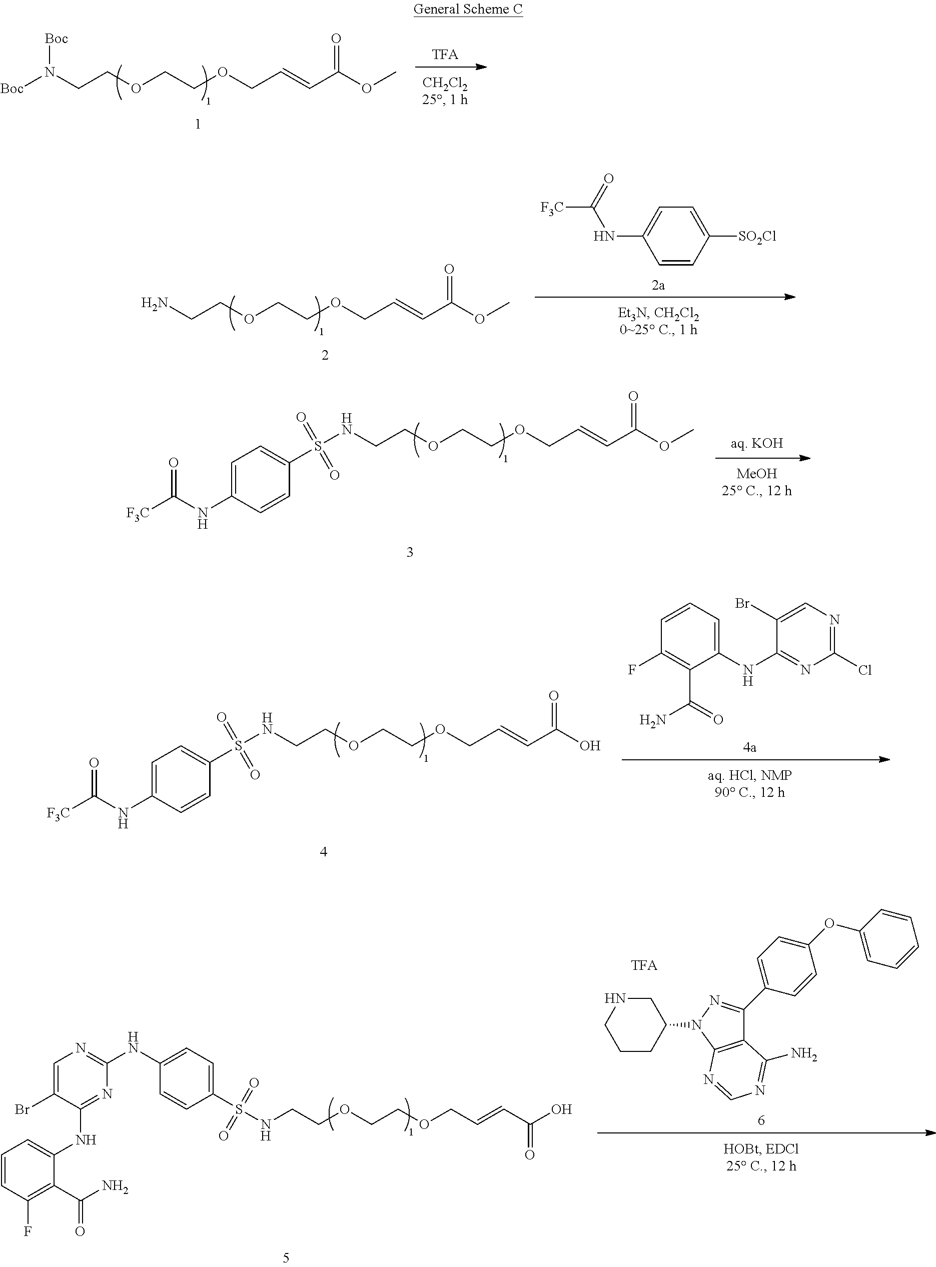

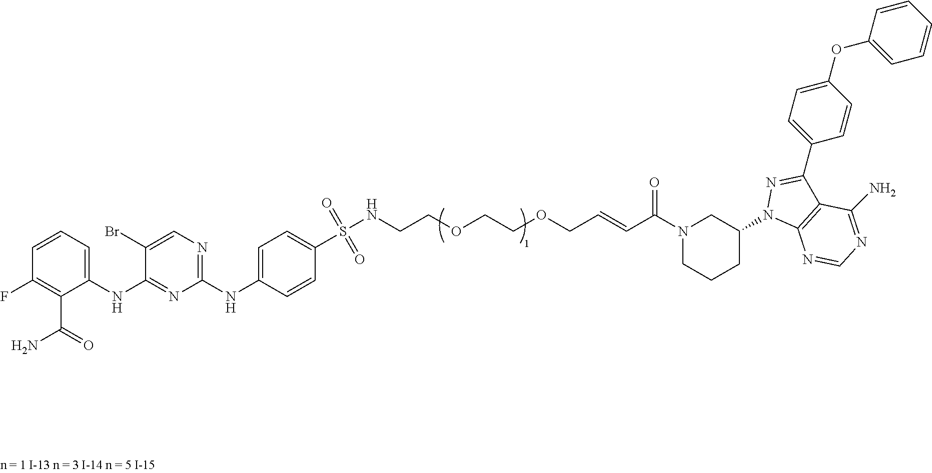

### 13.1 Preparation of Compound 2

A mixture of methyl (E)-4-[2-[2-[bis(tert-butoxycarbonyl)amino]ethoxy]ethoxy]but-2-enoate (0.35 g, 0.87 mmol, 1.0 equiv) in CH₂Cl₂ (4 mL) and TFA (2 mL) was stirred at 25° C. for 1 h. The reaction mixture was concentrated in vacuo to afford methyl (E)-4-[2-(2-aminoethoxy)ethoxy]but-2-enoate (0.18 g, crude, TFA) as a yellow oil. ¹HNMR (400 MHZ, DMSO-d₆): δ 7.92-7.75 (m, 3H), 6.93-6.88 (m, 1H), 6.05-6.01 (m, 1H), 4.19-4.16 (m, 2H), 3.66 (s, 3H), 3.63-3.58 (m, 6H), 3.07-2.90 (m, 2H).

### 13.2 Preparation of Compound 3

To a solution of methyl (E)-4-[2-(2-aminoethoxy)ethoxy]but-2-enoate (0.18 g, 0.57 mmol, TFA, 1.0 equiv) and 4-[(2,2,2-trifluoroacetyl)amino]benzenesulfonyl chloride (0.25 g, 0.85 mmol, 1.5 equiv) in CH₂Cl₂ (4 mL) was added Et₃N(0.28 g, 2.84 mmol, 5.0 equiv) at 0° C. The reaction mixture was warmed to 25° C. and stirred for 1 h. The reaction mixture was diluted with water (10 mL) and extracted with CH₂Cl₂ (15 mL*3). The combined organic phase was washed with brine (80 mL), dried with anhydrous Na₂SO₄, filtered and concentrated in vacuo. The residue was purified by column chromatography on silica gel (Petroleum ether/Ethyl acetate=3/1 to 1/1) to afford methyl (E)-4-[2-[2-[[4-[(2,2,2-trifluoroacetyl)amino]phenyl]sulfonylamino]ethoxy]ethoxy]but-2-enoate (0.20 g, 0.44 mmol, 77% yield) as a yellow oil. ¹HNMR (400 MHZ, DMSO-d₆): 11.58 (s, 1H), 7.92-7.79 (m, 4H), 7.71 (t, 1H, J=6.0 Hz), 6.90-8.86 (m, 1H), 6.01-5.97 (m, 1H), 4.17-4.08 (m, 2H), 3.66 (s, 3H), 3.52-3.44 (m, 4H), 3.39 (t, 2H, J=6.0 Hz), 2.93-2.89 (m, 2H). LC-MS: MS (ES⁺): m/z=455.1 [M+H⁺].

### 13.3 Preparation of Compound 4

A mixture of methyl (E)-4-[2-[2-[[4-[(2,2,2-trifluoroacetyl)amino]phenyl]sulfonylamino]ethoxy]ethoxy]but-2-enoate (0.20 g, 0.44 mmol, 1.0 equiv) in 1 Naq. KOH (2 mL) and MeOH (2 mL) was warmed to 25° C. and stirred for 12 h. The pH of the reaction mixture was adjusted to 2 by addition of 1 N aq. HCl and the mixture was concentrated in vacuo to afford (E)-4-[2-[2-[(4-aminophenyl)sulfonylamino]ethoxy]ethoxy]but-2-enoic acid (0.15 g, crude) as a yellow oil. LC-MS: MS (ES⁺): m/z=345.0 [M+H⁺].

### 13.4 Preparation of Compound 5

A mixture of (E)-4-[2-[2-[(4-aminophenyl)sulfonylamino]ethoxy]ethoxy]but-2-enoic acid (150 mg, 0.44 mmol, 1.0 equiv), 2-[(5-bromo-2-chloro-pyrimidin-4-yl)amino]-6-fluoro-benzamide (151 mg, 0.44 mmol, 1.0 equiv) and 12 N aq. HCl (15.9 mg, 0.44 mmol, 1.0 equiv) in NMP (2 mL) was heated to 90° C. and stirred for 12 h. The mixture was purified by prep-HPLC (column: Phenomenex luna C18 150*40 mm* 15 μm; mobile phase:

[water (0.1% TFA)]; B %: 24%-54%,11 min) to afford (E)-4-[2-[2-[[4-[[5-bromo-4-(2-carbamoyl-3-fluoro-anilino) pyrimidin-2-yl]amino]phenyl]sulfonylamino]ethoxy]ethoxy]but-2-enoic acid (100 mg, 0.15 mmol, 35% yield) as a yellow oil. ¹H NMR (400 MHZ, DMSO-d₆): δ 10.11 (s, 1H), 9.94 (s, 1H), 8.38 (s, 1H), 8.27-8.05 (m, 3H), 7.83 (d, 2H, J=8.8 Hz), 7.63 (d, 2H, J=8.8 Hz), 7.56-7.44 (m, 2H), 7.09 (t, 1H, J=9.2 Hz), 6.80-6.76 (m, 2H), 5.92-5.88 (m, 1H), 4.14-4.05 (m, 2H), 3.51-3.45 (m, 4H), 3.39 (t, 2H, J=5.6 Hz), 2.89-2.85 (m, 2H), 2.07 (s, 1H). LC-MS: MS (ES⁺): m/z=653.0 [M+H⁺].

### 13.5 Preparation of Compound I-13

To a mixture of (E)-4-[2-[2-[[4-[[5-bromo-4-(2-carbamoyl-3-fluoro-anilino) pyrimidin-2-yl]amino]phenyl]sulfonylamino]ethoxy]ethoxy]but-2-enoic acid (100 mg, 0.15 mmol, 1.0 equiv) and 3-(4-phenoxyphenyl)-1-[(3R)-3-piperidyl]pyrazolo[3,4-d]pyrimidin-4-amine (77.0 mg, 0.15 mmol, 1.0 equiv, TFA salt) in DMF (1 mL) were added HOBt (31.0 mg, 0.23 mmol, 1.5 equiv), DIPEA (158 mg, 1.2 mmol, 213 mL, 8.0 equiv) and EDCI (44.0 mg, 0.23 mmol, 1.5 equiv) at 25° C. The reaction mixture was stirred at 25° C. for 12 h. The mixture was purified by prep-HPLC (column: Unisil 3-100 C18 Ultra 150*50 mm*3 μm; mobile phase: [water (0.225% FA)-ACN]; B %: 45%-75%, 10 min) to afford 2-[[2-[4-[2-[2-[(E)-4-[(3R)-3-[4-amino-3-(4-phenoxyphenyl) pyrazolo[3,4-d]pyrimidin-1-yl]-1-piperidyl]-4-oxo-but-2-enoxy]ethoxy]ethylsulfamoyl]anilino]-5-bromo-pyrimidin-4-yl]amino]-6-fluoro-benzamide (37.6 mg, 0.04 mmol, 24% yield, 98% purity) as an off-white solid. ¹H NMR (400 MHZ, DMSO-d₆): δ 10.11 (s, 1H), 9.93 (s, 1H), 8.36 (s, 1H), 8.26-8.20 (m, 2H), 8.16 (s, 1H), 8.10 (s, 1H), 7.83 (d, 2H, J=7.6 Hz), 7.64 (s, 5H), 7.54-7.47 (m, 2H), 7.43 (t, 2H, J=7.6 Hz), 7.20-7.05 (m, 7H), 6.65 (s, 1H), 6.56-6.42 (m, 1H), 4.75-4.62 (m, 1H), 4.20-3.92 (m, 4H), 3.50 (s, 2H), 3.42-3.38 (m, 4H), 3.25-3.04 (m, 2H), 2.91-2.78 (m, 2H), 2.28-2.20 (m, 1H), 2.14-2.07 (m, 1H), 1.98-1.86 (m, 1H), 1.60-1.50 (m, 1H). LC-MS: MS (ES⁺): RT=2.323 min, m/z=1023.3 [M+H⁺]; LC-MS METHOD 25.

### Example 14—Synthesis of Compound I-14 (Synthesized According to General Scheme C in a Similar Manner to Compound I-13)

2-[[2-[4-[2-[2-[2-[2-[(E)-4-[(3R)-3-[4-amino-3-(4-phenoxyphenyl) pyrazolo[3,4-d]pyrimidin-1-yl]-1-piperidyl]-4-oxo-but-2-enoxy]ethoxy]ethoxy]ethoxy]ethylsulfamoyl]anilino]-5-bromo-pyrimidin-4-yl]amino]-6-fluoro-benzamide (55.5 mg, 0.05 mmol, 37% yield, 96% purity) as a yellow solid. ¹H NMR (400 MHZ, DMSO-d₆): δ 10.12 (s, 1H), 9.90 (s, 1H), 8.37 (s, 1H), 8.30-8.20 (m, 2H), 8.15-8.05 (m, 2H), 7.88-7.80 (m, 2H), 7.69-7.60 (m, 4H), 7.55-7.40 (m, 4H), 7.22-7.04 (m, 6H), 6.67-6.39 (m, 2H), 4.80-3.85 (m, 5H), 3.80-3.42 (m, 14H), 3.35-3.00 (m, 4H), 2.95-2.80 (m, 2H), 2.30-2.20 (m, 1H), 2.18-2.07 (m, 1H), 2.00-1.88 (m, 1H), 1.66-1.49 (m, 1H). LC-MS: MS (ES⁺): RT=2.340 min, m/z=556.3 [1/2M+H⁺]; LCMS METHOD 25.

### Example 15—Synthesis of Compound I-15 (Synthesized from 5 Below According to General Scheme C in a Similar Manner to Compound I-13)

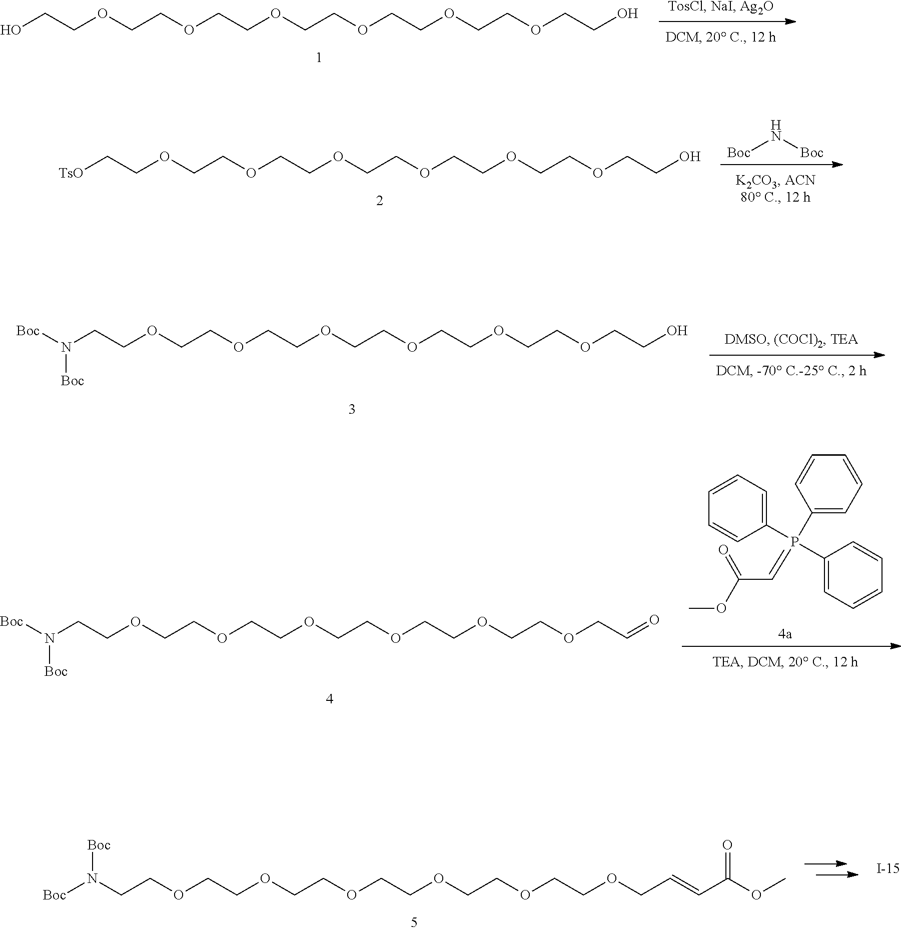

### 15.1 Preparation of Compound 2

To a solution of 2-[2-[2-[2-[2-[2-(2-hydroxyethoxy)ethoxy]ethoxy]ethoxy]ethoxy]ethoxy]ethanol (8 g, 24.5 mmol, 1 equiv) in DCM (100 mL) at 0° C. was added Ag₂O (8.52 g, 36.8 mmol, 1.5 equiv), NaI (4.04 g, 27 mmol, 1.1 equiv) and TosCl (4.67 g, 24.5 mmol, 1 equiv). The mixture was stirred at 20° C. for 12 h. The reaction mixture was filtered and concentrated. The residue was purified by prep-HPLC (column: Phenomenex Synergi Max-RP 250*80 mm*10 μm; mobile phase: [water (10 mM NH₄HCO₃)-CAN]; B %: 25%-55%, 20 min) to give 2-[2-[2-[2-[2-[2-(2-hydroxyethoxy)ethoxy]ethoxy]ethoxy]ethoxy]ethoxy]ethyl 4-methylbenzenesulfonate (6.5 g, 55% yield) as a yellow oil. ¹H NMR (400 MHZ, CDCl₃): δ 7.81 (d, J=8.4 Hz, 2H), 7.36 (d, J=8.4 Hz, 2H), 4.24-4.09 (m, 2H), 3.73-3.59 (m, 26H), 3.09-2.96 (m, 1H), 2.46 (s, 3H).

### 15.2 Preparation of Compound 3

To a solution of 2-[2-[2-[2-[2-[2-(2-hydroxyethoxy)ethoxy]ethoxy]ethoxy]ethoxy]ethoxy]ethyl 4-methylbenzenesulfonate (6.3 g, 13.1 mmol, 1 equiv) in ACN(60 mL) was added K₂CO₃ (3.62 g, 26.2 mmol, 2 equiv) and tert-butyl N-tert-butoxycarbonylcarbamate (4.27 g, 19.7 mmol, 1.5 equiv). The mixture was stirred at 80° C. for 12 h. The reaction mixture was filtered and concentrated. The residue was purified by prep-HPLC (column: Kromasil Eternity XT 250*80 mm*10 μm; mobile phase: [water (10 mM NH₄HCO₃)-ACN]; B %: 35%-65%, 15 min) to give tert-butyl N-tert-butoxycarbonyl-N-[2-[2-[2-[2-[2-[2-(2-hydroxyethoxy)ethoxy]ethoxy]ethoxy]ethoxy]ethoxy]ethyl]carbamate (5 g, 72% yield) as a yellow oil. ¹H NMR (400 MHZ, CDCl₃): δ 3.83-3.61 (m, 28H), 1.52 (s, 17H).

### 15.3 Preparation of Compound 4

To a solution of DMSO (7.43 g, 95.12 mmol, 10 equiv) in DCM (60 mL) was added (COCl)₂ (6.04 g, 47.6 mmol, 5 equiv) at −70° C. and the mixture was stirred for 0.5 h. Then tert-butyl N-tert-butoxycarbonyl-N-[2-[2-[2-[2-[2-[2-(2-hydroxyethoxy)ethoxy]ethoxy]ethoxy]ethoxy]ethoxy]ethyl]carbamate (5 g, 9.5 mmol, 1 equiv) was added dropwise and the mixture was stirred at −70° C. for 0.5 h. After that TEA (9.63 g, 95.1 mmol, 10 equiv) was added and the mixture was stirred at −70° C. for another 0.5 h. The mixture was slowly warmed to 25° C. and stirred at 25° C. for 0.5 h. The reaction mixture was concentrated under reduced pressure to give crude tert-butyl N-tert-butoxycarbonyl-N-[2-[2-[2-[2-[2-[2-(2-oxoethoxy)ethoxy]ethoxy]ethoxy]ethoxy]ethoxy]ethyl]carbamate (4.9 g, crude) as a yellow oil which was used directly for next step.

### 15.4 Preparation of Compound 5

To a solution of tert-butyl N-tert-butoxycarbonyl-N-[2-[2-[2-[2-[2-[2-(2-oxoethoxy)ethoxy]ethoxy]ethoxy]ethoxy]ethoxy]ethyl]carbamate (4.9 g, 9.36 mmol, 1 equiv) in DCM (50 mL) was added TEA (2.84 g, 28 mmol, 3 equiv) and methyl 2-(triphenyl-λ⁵-phosphanylidene) acetate (3.75 g, 11.2 mmol, 1.2 equiv). The mixture was stirred at 20° C. for 12 h and the solvent was concentrated. The residue was purified by prep-HPLC (column: Waters Xbridge BEH C18 250*50 mm*10 μm; mobile phase: [water (10 mM NH₄HCO₃)-ACN]; B %: 40%-70%,20 min), (column: Phenomenex luna C18 250*50 mm*10 μm; mobile phase: [water (10 mM NH₄HCO₃)-ACN]; B %: 40%-65%,25 min) and (column: Waters Xbridge 150*25 mm*5 μm; mobile phase: [water (10 mM NH₄HCO₃)-ACN]; B %: 48%-78%,9 min) to give the desired compound methyl (E)-4-[2-[2-[2-[2-[2-[2-[bis(tert-butoxycarbonyl)amino]ethoxy]ethoxy]ethoxy]ethoxy]ethoxy]ethoxy]but-2-enoate (1.8 g, 33% yield) as a yellow oil. ¹H NMR (400 MHZ, CDCl₃): δ 7.05-6.89 (m, 1H), 6.13-6.08 (m, 1H), 4.22-4.20 m, 2H), 3.82-3.78 (m, 2H), 3.77-3.74 (m, 3H), 3.71-3.61 (m, 20H), 1.52 (s, 18H).

### 15.5 Preparation of Compound I-15

2-[2-[4-[2-[2-[2-[2-[2-[2-[(E)-4-[(3R)-3-[4-amino-3-(4-phenoxyphenyl) pyrazolo[3,4-d]pyrimidin-1-yl]-1-piperidyl]-4-oxo-but-2-enoxy]ethoxy]ethoxy]ethoxy]ethoxy]ethoxy]ethylsulfamoyl]anilino]-5-bromo-pyrimidin-4-yl]amino]-6-fluoro-benzamide (16 mg, 21% yield) as a white solid. ¹H NMR (400 MHZ, CD₃OD): δ 8.63-7.88 (m, 4H), 7.87-7.66 (m, 6H), 7.55-7.39 (m, 3H), 7.19-7.09 (m, 4H), 6.99 (s, 1H), 6.85-6.48 (m, 2H), 4.58 (s, 1H), 4.33-4.02 (m, 4H), 3.66-3.45 (m, 24H), 3.05 (s, 2H), 2.22 (s, 2H), 1.69 (s, 1H), 1.40-1.32 (m, 1H). LC-MS: MS (ES⁺): RT=2.354 min, m/z=1197.3 [M+H⁺]. LC-MS METHOD 25.

### Example 16—Synthesis of Compound I-16

### Part I

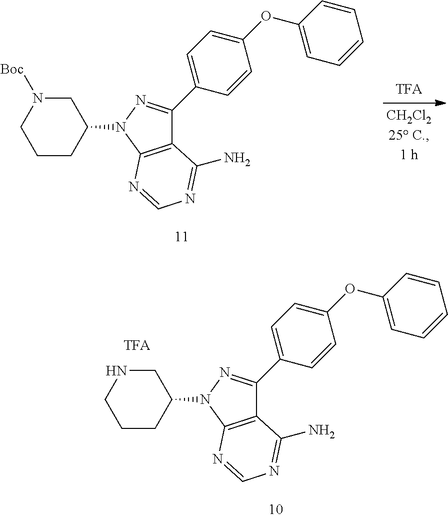 [SMILES: CC(C)(C)OC(=O)N1CCC[C@@H](n2nc(-c3ccc(Oc4ccccc4)cc3)c3c(N)ncnc32)C1 ; Nc1ncnc2c1c(-c1ccc(Oc3ccccc3)cc1)nn2[C@@H]1CCCNC1]

### Part II

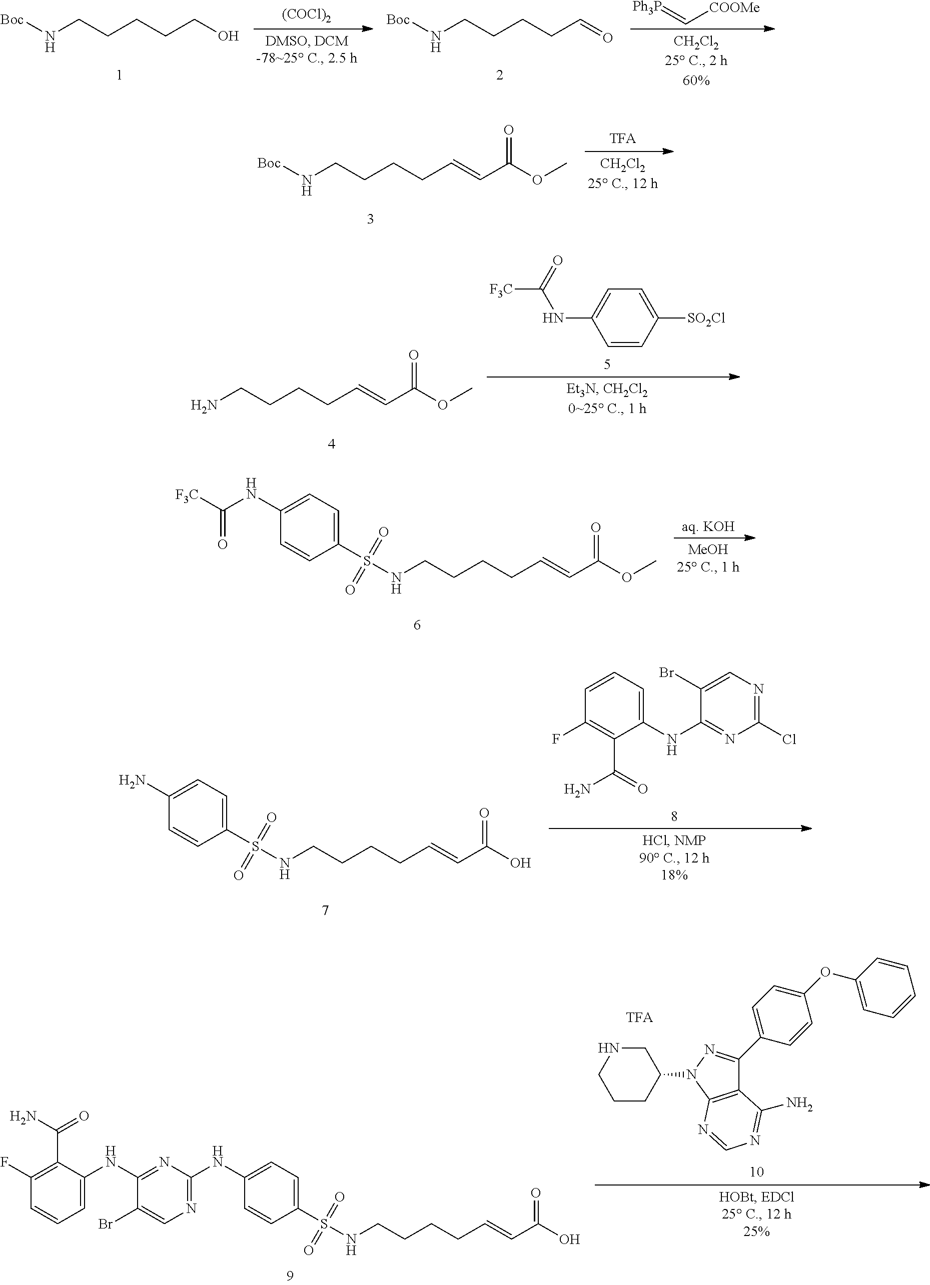

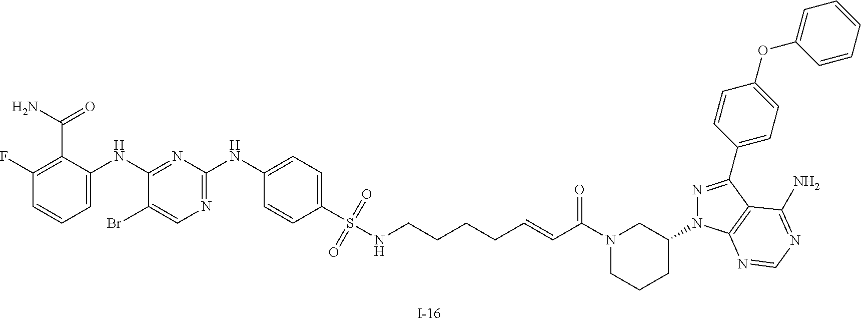

### 16.1 Preparation of Compound 2

To a solution of (COCl)₂ (4.25 g, 33.5 mmol, 2.0 equiv) in dry DCM (55 mL) was added dry DMSO (5.23 g, 66.9 mmol, 5.23 mL, 4.0 equiv) at −78° C. The reaction mixture was stirred at −78° C. for 0.5 h. To the reaction mixture was added a solution of tert-butyl N-(5-hydroxypentyl) carbamate (3.40 g, 16.7 mmol, 1.0 equiv) in DCM (7 mL) and the mixture was stirred at −78° C. for 0.5 h. To the reaction mixture was added Et₃N(10.2 g, 100 mmol, 6.0 equiv) and the mixture was stirred at −78° C. for 0.5 h. The reaction mixture was warmed to 25° C. and stirred for 1 h. The reaction mixture was diluted with water (80 mL) and extracted with CH₂Cl₂ (45 mL*2). The combined organic phase was dried with anhydrous Na₂SO₄, filtered and concentrated in vacuo to afford tert-butyl N-(5-oxopentyl) carbamate (3.37 g, crude) as a yellow oil. ¹HNMR (400 MHZ, CDCl₃): δ 9.79 (t, 1H, J=2.0 Hz), 6.89-6.69 (m, 1H), 4.97-4.77 (m, 1H), 4.62-4.52 (m, 1H), 3.84-3.79 (m, 2H), 3.60-3.55 (m, 2H), 3.18-3.11 (m, 4H), 1.68 (s, 9H).

### 16.2 Preparation of Compound 3

A mixture of tert-butyl N-(5-oxopentyl) carbamate (3.37 g, 16.7 mmol, 1.0 equiv) and methyl 2-(triphenyl-2.5-phosphanylidene) acetate (6.72 g, 20.1 mmol, 1.2 equiv) in CH₂Cl₂ (40 mL) was stirred at 25° C. for 2 h. The reaction mixture was concentrated in vacuo. The residue was purified by column chromatography on silica gel (Petroleum ether/Ethyl acetate=100/1 to 20/1) to afford methyl €-7-(tert-butoxycarbonylamino) hept-2-enoate (2.60 g, 10.1 mmol, 60% yield) as a colorless oil. ¹HNMR (400 MHZ, CDCl₃): δ 7.02-6.92 (m, 1H), 5.85 (td, 1H, J=1.6, 15.6 Hz), 4.57-4.45 (m, 1H), 3.75 (s, 3H), 3.21-3.05 (m, 2H), 2.30-2.19 (m, 2H), 1.54-1.50 (m, 4H), 1.47 (s, 9H).

### 16.3 Preparation of Compound 4

A mixture of methyl (E)-7-(tert-butoxycarbonylamino) hept-2-enoate (600 mg, 2.33 mmol, 1 equiv) in DCM (8 mL) and TFA (4 mL) was stirred at 25° C. for 1 h. The reaction mixture was concentrated in vacuo to afford methyl (E)-7-aminohept-2-enoate (632 mg, crude, TFA salt) as a colorless oil.

### 16.4 Preparation of Compound 6

To a solution of methyl (E)-7-aminohept-2-enoate (632 mg, 2.33 mmol, 1.0 equiv, TFA salt) and 4-[(2,2,2-trifluoroacetyl)amino]benzenesulfonyl chloride (1.01 g, 3.50 mmol, 1.5 equiv) in DCM (12 mL) was added Et₃N(1.18 g, 11.6 mmol, 1.62 mL, 5.0 equiv) at 0° C. The reaction mixture was warmed to 25° C. and stirred for 1 h. The reaction mixture was diluted with DCM (25 mL), washed with water (15 mL), dried with anhydrous Na₂SO₄, filtered and concentrated in vacuo. The residue was purified by column chromatography on silica gel (Petroleum ether/Ethyl acetate=10/1 to 2/1) to afford methyl (E)-7-[[4-[(2,2,2-trifluoroacetyl) amino]phenyl]sulfonylamino]hept-2-enoate (880 mg, 1.79 mmol, 83% purity) as a white solid. ¹HNMR (400 MHZ, CDCl₃): δ 8.61 (brs, 1H), 7.91 (d, 2H, J=8.8 Hz), 7.79 (d, 2H, J=8.8 Hz), 6.85-6.74 (m, 1H), 5.75 (d, 1H, J=15.6 Hz), 4.63 (t, 1H, J=6.0 Hz), 3.76 (s, 3H), 3.02 (q, 2H, J=6.4 Hz), 2.21-2.10 (m, 2H), 1.51-1.34 (m, 4H). LC-MS: MS (ES⁺): m/z=431.0 [M+Na⁺].

### 16.5 Preparation of Compound 7

A mixture of methyl (E)-7-[[4-[(2,2,2-trifluoroacetyl)amino]phenyl]sulfonylamino]hept-2-enoate (300 mg, 0.74 mmol, 83% purity, 1.0 equiv) and aq. KOH (1 M, 3 mL, 4.08 equiv) in MeOH (3 mL) was stirred at 25° C. for 1 h. The pH of the reaction mixture was adjusted to 7 by addition of 1 N HCl. The mixture was concentrated in vacuo to afford (E)-7-[(4-aminophenyl)sulfonylamino]hept-2-enoic acid (220 mg, crude) as a yellow oil. LC-MS: MS (ES⁺): m/z=281.0 [M-OH—].

### 16.6 Preparation of Compound 9

A mixture of (E)-7-[(4-aminophenyl)sulfonylamino]hept-2-enoic acid (0.22 g, 0.74 mmol, 1.0 equiv), 2-[(5-bromo-2-chloropyrimidin-4-yl)amino]-6-fluoro-benzamide (0.25 g, 0.74 mmol, 1.0 equiv) and aq. HCl (12 M, 61.5 mL, 1.0 equiv) in NMP (4 mL) was stirred at 90° C. for 12 h. The reaction mixture was purified by prep-HPLC (column: 3_Phenomenex Luna C18 75*30 mm*3 μm; mobile phase: [water (0.1% TFA)-€]; B %: 42%-62%,7 min) to afford (E)-7-[[4-[[5-bromo-4-(2-carbamoyl-3-fluoro-anilino) pyrimidin-2-yl]amino]phenyl]sulfonylamino]hept-2-enoic acid (80.0 mg, 132 mmol, 18% yield) as a yellow solid. ¹HNMR (400 MHZ, DMSO-d₆): 11.12 (1H, brs), 10.10 (s, 1H), 9.92 (s, 1H), 8.38 (s, 1H), 8.27-8.20 (m, 1H), 8.18-8.08 (m, 2H), 7.90-7.80 (m, 2H), 7.67-7.59 (m, 2H), 7.56-7.46 (m, 1H), 7.39 (t, 1H, J=6.0 Hz), 7.10-7.08 (m, 1H), 6.81-6.71 (m, 1H), 5.74-5.70 (m, 1H), 2.74-2.67 (m, 2H), 2.14-2.06 (m, 2H), 1.43-1.31 (m, 4H).

### 16.7 Preparation of Compound 10

A mixture of tert-butyl(3R)-3-[4-amino-3-(4-phenoxyphenyl) pyrazolo[3,4-d]pyrimidin-1-yl]piperidine-1-carboxylate (30.0 mg, 61.7 mmol, 1.0 equiv) in CH₂Cl₂ (2 mL) and TFA (1 mL) was stirred at 25° C. for 1 h. The reaction mixture was filtered and concentrated in vacuo to afford 3-(4-phenoxyphenyl)-1-[(3R)-3-piperidyl]pyrazolo[3,4-d]pyrimidin-4-amine (24.0 mg, crude, TFA salt) as a yellow oil. LC-MS: MS (ES⁺): RT=0.633 min, m/z=387.1 [M+H⁺]; LC-MS METHOD 40.

### 16.8 Preparation of Compound I-16

To a solution of (E)-7-[[4-[[5-bromo-4-(2-carbamoyl-3-fluoro-anilino) pyrimidin-2-yl]amino]phenyl]sulfonylamino]hept-2-enoic acid (40.0 mg, 65.9 mmol, 1.0 equiv) and 3-(4-phenoxyphenyl)-1-[(3R)-3-piperidyl]pyrazolo[3,4-d]pyrimidin-4-amine (41.0 mg, 0.82 mmol, 1.24 equiv, TFA salt) in DMF (3 mL) was added HOBt (13.3 mg, 0.99 mmol, 1.5 equiv), EDCI (18.9 mg, 0.99 mmol, 1.5 equiv), DIPEA (68.1 mg, 0.53 mmol, 8.0 equiv). The mixture was stirred at 25° C. for 12 h. The residue was purified by prep-HPLC (column: Unisil 3-100 C18 Ultra 150*50 mm*3 μm; mobile phase: [water (0.225% FA)-ACN]; B %: 48%-78%, 10 min) to afford 2-[[2-[4-[[(E)-7-[(3R)-3-[4-amino-3-(4-phenoxyphenyl) pyrazolo[3,4-d]pyrimidin-1-yl]-1-piperidyl]-7-oxo-hept-5-enyl]sulfamoyl]anilino]-5-bromo-pyrimidin-4-yl]amino]-6-fluoro-benzamide (16.3 mg, 16.4 mmol, 25% yield, 98% purity) was obtained as an off-white solid. ¹H NMR (400 MHZ, CDCl₃) δ 11.86-11.27 (m, 1H), 8.47-8.07 (m, 4H), 7.82-7.61 (m, 6H), 7.43-7.40 (m, 3H), 7.22-7.03 (m, 5H), 6.99-5.34 (m, 5H), 5.13-3.75 (m, 2H), 3.42-2.77 (m, 3H), 2.42-1.88 (m, 9H), 1.68-1.06 (m, 6H). LC-MS: MS (ES⁺): RT=1.751 min, m/z=976.9 [M+H⁺]; LC-MS METHOD 40.

### Example 17—Synthesis of Compound I-17

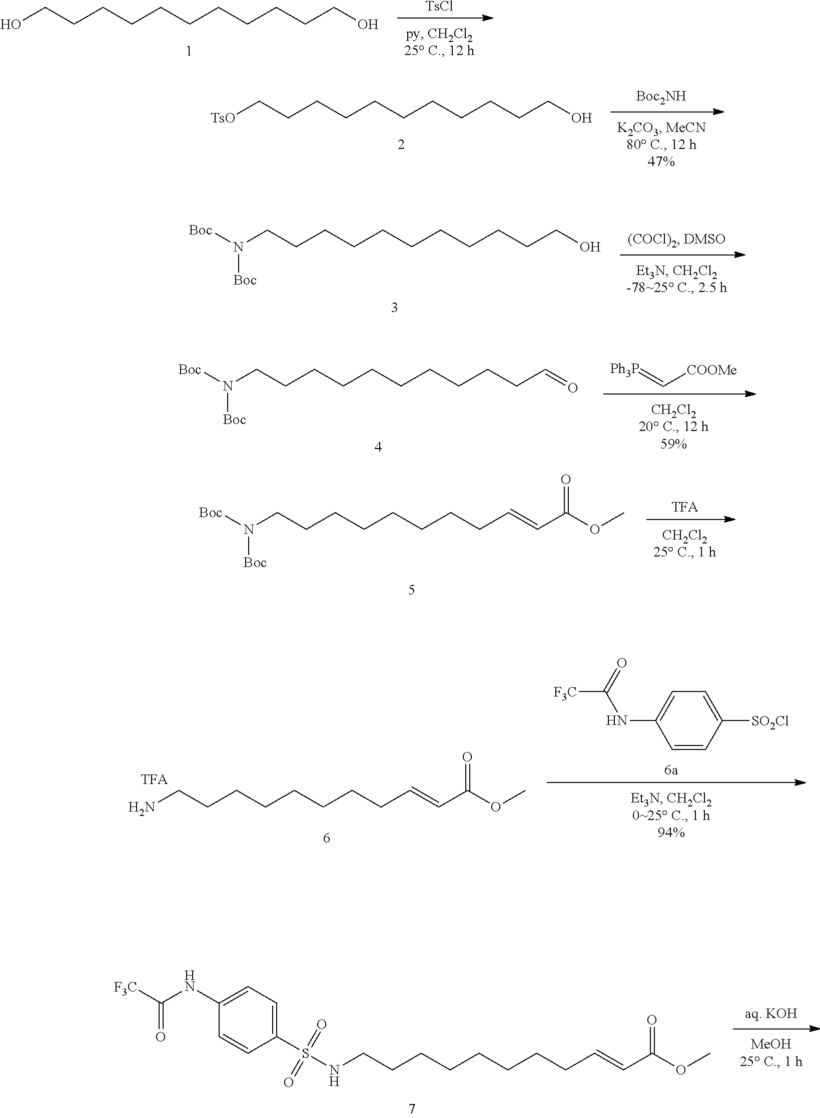

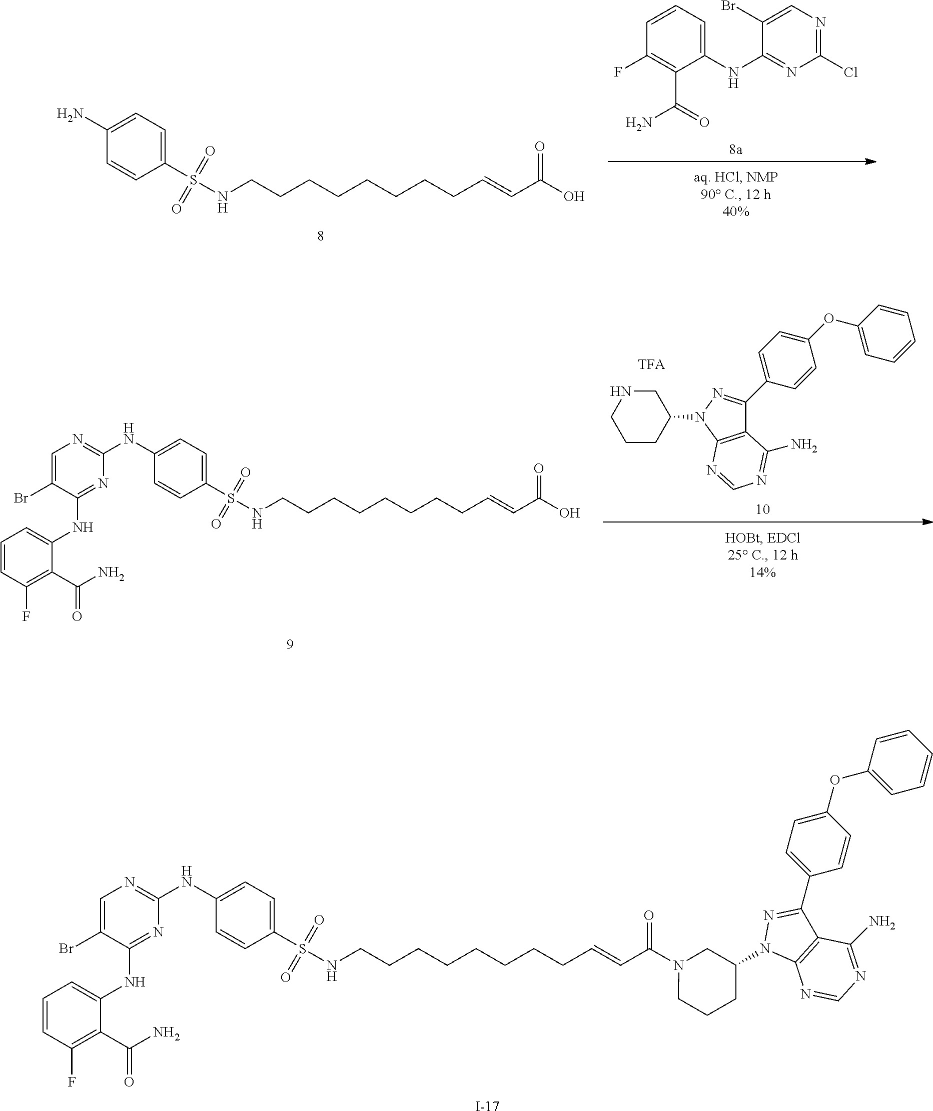

### 17.1 Preparation of Compound 2

To a solution of nonane-1,9-diol (20.0 g, 124 mmol, 2.5 equiv) in CH₂Cl₂ (200 mL) were added TsCl (9.52 g, 49.9 mmol, 1.0 equiv) and pyridine (9.87 g, 124 mmol, 10.0 mL, 2.5 equiv) at 25° C. The mixture was stirred at 25° C. for 12 h. The reaction mixture was diluted with water (50 mL) and extracted with ethyl acetate (60 mL*3). The combined organic phase was washed with brine (100 mL), dried with anhydrous Na₂SO₄, filtered and concentrated in vacuo. The residue was purified by column chromatography on silica gel (Petroleum ether/Ethyl acetate=3/1 to 1/2) to afford 9-hydroxynonyl 4-methylbenzenesulfonate (15.0 g, crude) as a white solid. ¹HNMR (400 MHZ, CD₃Cl): 7.87-7.66 (m, 2H), 7.33 (d, 2H, J=8.0 Hz), 4.03-3.96 (m, 2H), 3.61-3.58 (m, 2H), 2.51-2.36 (m, 3H), 1.25-1.21 (m, 6H), 1.26-1.21 (m, 8H).

### 17.2 Preparation of Compound 3]

To a solution of 9-hydroxynonyl 4-methylbenzenesulfonate (15.0 g, 47.7 mmol, 1.0 equiv) in CH₃CN(150 mL) were added tert-butyl N-tert-butoxycarbonylcarbamate (12.4 g, 57.2 mmol, 1.2 equiv) and K₂CO₃ (19.7 g, 143 mmol, 3.0 equiv) at 25° C. The mixture was stirred at 80° C. for 12 h. The reaction mixture was concentrated in vacuo. The residue was diluted with water (50 mL) and extracted with ethyl acetate (45 mL*3). The combined organic phase was washed with brine (100 mL), dried with anhydrous Na₂SO₄, filtered and concentrated in vacuo. The residue was purified by column chromatography on silica gel (Petroleum ether/Ethyl acetate=3/1 to 1/1) to afford tert-butyl N-tert-butoxycarbonyl-N-(9-hydroxynonyl) carbamate (8.00 g, 22.3 mmol, 47% yield) as a yellow oil. ¹HNMR (400 MHZ, CD₃Cl): □3.63 (t, 2H, J=6.4 Hz), 3.56-3.51 (m, 2H), 1.60-1.52 (m, 4H), 1.51-1.46 (m, 18H), 1.35-1.27 (m, 10H).

17.3 Preparation of Compound 4

To a solution of oxalyl chloride (2.82 g, 22.2 mmol, 1.95 mL, 2.0 equiv) in dry CH₂Cl₂ (40 mL) was added dry DMSO (2.61 g, 33.3 mmol, 2.61 mL, 3.0 equiv) dropwise at −78° C. The mixture was stirred at −78° C. for 0.5 h. To the mixture was added a solution tert-butyl N-tert-butoxycarbonyl-N-(9-hydroxynonyl) carbamate (4.00 g, 11.1 mmol, 1.0 equiv) in CH₂Cl₂ (10 mL) dropwise at −78° C. The mixture was stirred at −78° C. for 0.5 h. To the mixture was added triethylamine (5.63 g, 55.6 mmol, 7.74 mL, 5.0 equiv) dropwise at −78° C. The mixture was stirred at −78° C. for 0.5 h. The mixture was warmed to 25° C. and stirred for 1 h. The reaction mixture was diluted with water (10 mL) and extracted with CH₂Cl₂ (15 mL*3). The combined organic phase was washed with water (35 mL*3) and brine (80 mL), dried with anhydrous Na₂SO₄, filtered and concentrated in vacuo to afford tert-butyl N-tert-butoxycarbonyl-N-(9-oxononyl) carbamate (3.98 g, crude) as a yellow solid. ¹H NMR (400 MHz, DMSO-d₆): □9.01-8.49 (m, 1H), 2.59 (1, 2H, J=7.2 Hz), 1.60-1.50 (m, 2H), 0.69-0.55 (m, 22H), 0.43-0.37 (m, 8H).

### 17.4 Preparation of Compound 5

A mixture of tert-butyl N-tert-butoxycarbonyl-N-(9-oxononyl) carbamate (3.98 g, 11.1 mmol, 1.0 equiv) and methyl 2-(triphenyl-2.5-phosphanylidene) acetate (4.47 g, 13.3 mmol, 1.2 equiv) in CH₂Cl₂ (40 mL) was stirred at 25° C. for 12 h. The reaction mixture was diluted with water (30 mL) and extracted with CH₂Cl₂ (45 mL*3). The combined organic phase was washed with brine (80 mL), dried with anhydrous Na₂SO₄, filtered and concentrated in vacuo. The residue was purified by column chromatography on silica gel (Petroleum ether/Ethyl acetate=5/1 to 3/1) to afford methyl (E)-11-[bis(tert-butoxycarbonyl)amino]undec-2-enoate (2.70 g, 6.53 mmol, 58% yield) as a yellow oil. ¹H NMR (400 MHZ, DMSO-d₆): 6.98-6.94 (m, 1H), 5.83-5.79 (m, 1H), 3.74-3.69 (m, 3H), 3.58-3.49 (m, 2H), 2.25-2.09 (m, 2H), 1.58-1.44 (m, 22H), 1.31-1.26 (m, 8H).

### 17.5 Preparation of Compound 6

A mixture of methyl (E)-11-[bis(tert-butoxycarbonyl)amino]undec-2-enoate (0.30 g, 0.72 mmol, 1.0 equiv) in CH₂Cl₂ (6 mL) and TFA (3 mL) was stirred at 25° C. for 12 h. The reaction mixture was concentrated in vacuo to afford methyl (E)-11-aminoundec-2-enoate (0.24 g, crude, TFA) as a yellow oil. LC-MS: MS (ES⁺): m/z=214.1 [M+H⁺].

### 17.6 Preparation of Compound 7

To a solution of methyl (E)-11-aminoundec-2-enoate (0.24 g, 0.73 mmol, TFA, 1.0 equiv) and 4-[(2,2,2-trifluoroacetyl)amino]benzenesulfonyl chloride (0.32 g, 1.10 mmol, 1.5 equiv) in CH₂Cl₂ (4 mL) was added Et₃N(0.37 g, 3.67 mmol, 5.0 equiv) at 0° C. The reaction mixture was warmed to 25° C. and stirred for 1 h. The reaction mixture was diluted with water (10 mL) and extracted with CH₂Cl₂ (15 mL*3). The combined organic phase was washed with brine (20 mL), dried with anhydrous Na₂SO₄, filtered and concentrated in vacuo. The residue was purified by column chromatography on silica gel (Petroleum ether/Ethyl acetate=3/1 to 1/1) to afford methyl (E)-11-[4-[(2,2,2-trifluoroacetyl)amino]phenyl]sulfonylamino]undec-2-enoate (0.32 g, 0.69 mmol, 94% yield) as a yellow solid. ¹H NMR (400 MHZ, DMSO-d₆): δ 11.59 (s, 1H), 7.93-7.73 (m, 4H), 7.56 (t, 1H, J=6.0 Hz), 6.89-6.85 (m, 1H), 5.85 (d, J=15.6 Hz, 1H), 3.63 (s, 3H), 2.73-2.69 (m, 2H), 2.21-2.07 (m, 2H), 1.36-1.32 (m, 4H), 1.25-1.09 (m, 8H). LC-MS: MS (ES⁺): m/z=465.1 [M+H⁺].

### 17.7 Preparation of Compound 8

A mixture of methyl (E)-11-[[4-[(2,2,2-trifluoroacetyl)amino]phenyl]sulfonylamino]undec-2-enoate (270 mg, 0.58 mmol, 1.0 equiv) in 1 N aq. KOH (5 mL) and MeOH (5 mL) was warmed to 25° C. and stirred for 1 h. The pH of the reaction mixture was adjusted to 2 by addition of 1 N aq. HCl and the mixture was concentrated in vacuo. The residue was triturated with EtOH (5 mL). The mixture was filtered and the filtrate was concentrated to afford (E)-11-[(4-aminophenyl)sulfonylamino]undec-2-enoic acid (200 mg, crude) as a yellow oil. LC-MS: MS (ES⁺): RT=0.874 min, m/z=377.1 [M+Na⁺].

### 17.8 Preparation of Compound 9

A mixture of (E)-11-[(4-aminophenyl)sulfonylamino]undec-2-enoic acid (200 mg, 0.56 mmol, 1.0 equiv), 2-[(5-bromo-2-chloro-pyrimidin-4-yl)amino]-6-fluoro-benzamide (190 mg, 0.56 mmol, 1.0 equiv) and concentrated aq. HCl (20.6 mg, 0.56 mmol, 1.0 equiv) in NMP (3 mL) was heated to 90° C. and stirred for 12 h. The reaction mixture was filtered and the filtrate was concentrated in vacuo. The residue was purified by prep-HPLC (column: Phenomenex Synergi C18 150*25 mm*10 μm; mobile phase: [water (0.1% TFA)-ACN]; B %: 48%-78%,10 min) to afford (E)-11-[[4-[[5-bromo-4-(2-carbamoyl-3-fluoro-anilino) pyrimidin-2-yl]amino]phenyl]sulfonylamino]undec-2-enoic acid (150 mg, 0.23 mmol, 40% yield) as a yellow solid. ¹H NMR (400 MHZ, DMSO-d₆): □12.10 (brs, 1H), 10.12 (s, 1H), 9.93 (s, 1H), 8.38 (s, 1H), 8.24 (d, 1H, J=8.4 Hz), 8.19-8.05 (m, 2H), 7.87-7.80 (m, 2H), 7.62 (d, 2H, J=8.8 Hz), 7.56-7.46 (m, 1H), 7.35 (t, 1H, J=5.6 Hz), 7.08 (t, 1H, J=9.2 Hz), 6.80-6.76 (m, 1H), 5.78-5.67 (m, 1H), 2.70-2.67 (m, 2H), 2.23-2.02 (m, 2H), 1.37-1.32 (m, 4H), 1.19-1.14 (m, 8H).

### 17.9 Preparation of Compound I-17

To a solution of 3-(4-phenoxyphenyl)-1-[(3R)-3-piperidyl]pyrazolo[3,4-d]pyrimidin-4-amine (66.0 mg, 0.13 mmol, 1.17 equiv, TFA salt) and (E)-11-[[4-[[5-bromo-4-(2-carbamoyl-3-fluoro-anilino) pyrimidin-2-yl]amino]phenyl]sulfonylamino]undec-2-enoic acid (77.0 mg, 0.11 mmol, 1.0 equiv) in DMF (3 mL) were added HOBt (22.9 mg, 0.17 mmol, 1.5 equiv), EDCI (32.5 mg, 0.17 mmol, 1.5 equiv) and DIPEA (117 mg, 0.90 mmol, 157 mL, 8.0 equiv). The mixture was stirred at 25° C. for 12 h. The mixture was purified by prep-HPLC (column: Unisil 3-100 C18 Ultra 150*50 mm*3 μm; mobile phase: [water (0.225% FA)-ACN]; B %: 58%-88%, 10 min) to afford 2-[[2-[4-[(E)-11-[(3R)-3-[4-amino-3-(4-phenoxyphenyl) pyrazolo[3,4-d]pyrimidin-1-yl]-1-piperidyl]-11-oxo-undec-9-enyl]sulfamoyl]anilino]-5-bromo-pyrimidin-4-yl]amino]-6-fluoro-benzamide (22.0 mg, 0.02 mmol, 14% yield, 100% purity) as an off-white solid. ¹H NMR (400 MHZ, CD₃Cl): δ 11.65-11.42 (m, 1H), 8.44 (d, 1H, J=8.4 Hz), 8.32 (s, 1H), 8.24 (brs, 1H), 8.13 (brs, 1H), 7.83-7.71 (m, 4H), 7.64 (d, 2H, J=7.6 Hz), 7.48-7.37 (m, 3H), 7.22-7.06 (m, 5H), 6.94-5.99 (m, 6H), 4.97-3.98 (m, 3H), 3.65-2.85 (m, 4H), 2.71-2.42 (m, 3H), 2.34-2.00 (m, 4H), 1.80-1.66 (m, 1H), 1.52-1.33 (m, 4H), 1.23-1.15 (m, 8H). LC-MS: MS (ES⁺): RT=2.190 min, m/z=1033.3 [M+H⁺]; LC-MS METHOD 40.

### Example 18—Synthesis of Compound I-18

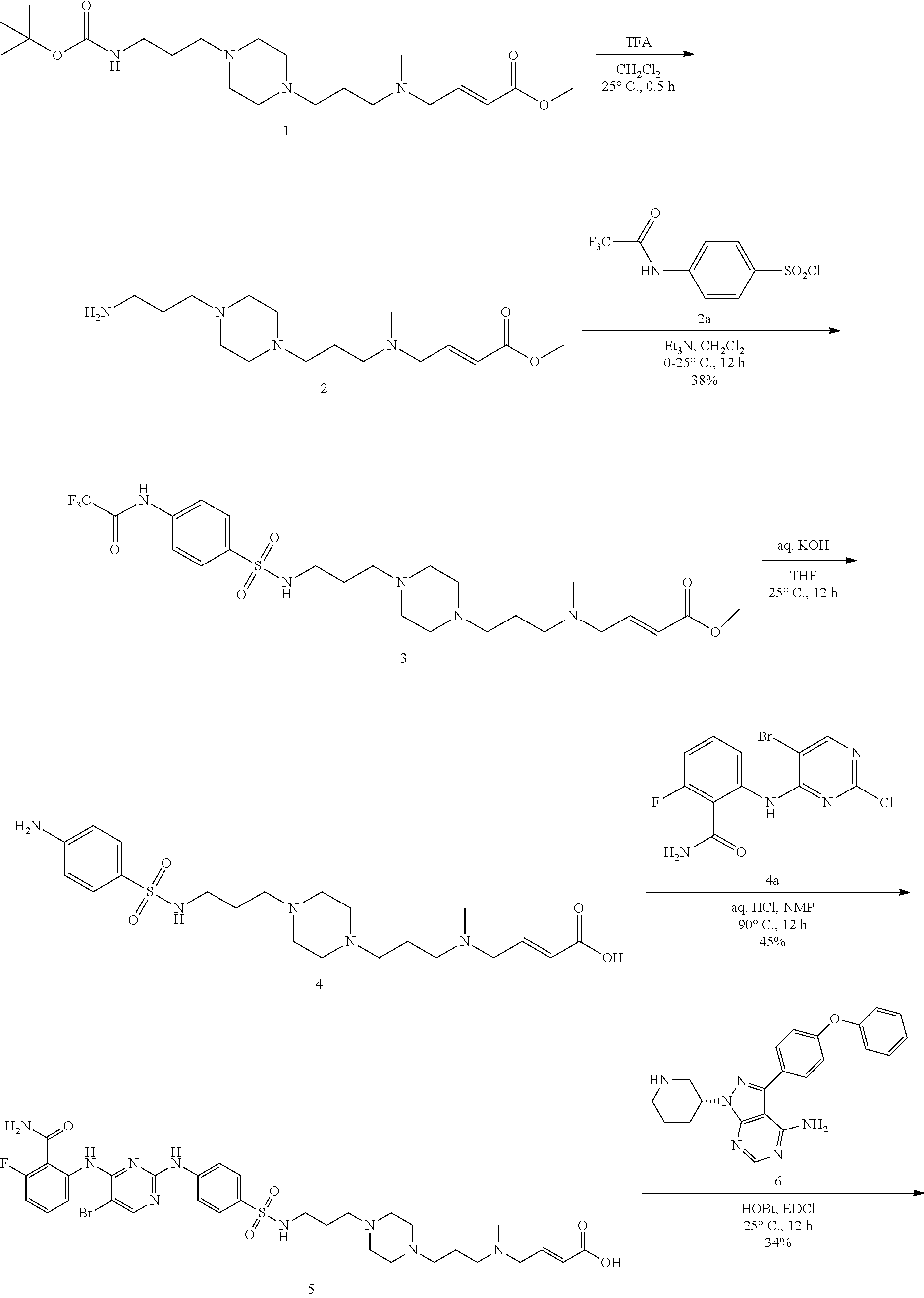

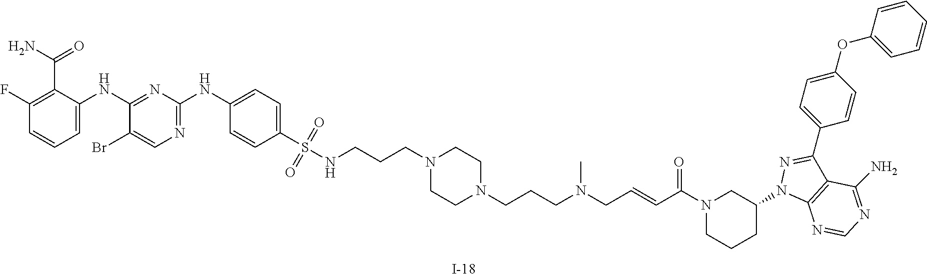

### 18.1 Preparation of Compound 2

A mixture of methyl (E)-4-[3-[4-[3-(tert-butoxycarbonylamino) propyl]piperazin-1-yl]propyl-methyl-amino]but-2-enoate (370 mg, 0.78 mmol, 1.0 equiv) in CH₂Cl₂ (4 mL) and TFA (2 mL) was stirred at 25° C. for 0.5 h. The reaction mixture was concentrated in vacuo to afford methyl (E)-4-[3-[4-(3-aminopropyl) piperazin-1-yl]propyl-methyl-amino]but-2-enoate (381 mg, crude, TFA salt) as a yellow oil. LC-MS: MS (ES⁺): RT=0.683 min, m/z=313.3 [M+H⁺].

### 18.2 Preparation of Compound 3

To a solution of 4-[(2,2,2-trifluoroacetyl)amino]benzenesulfonyl chloride (385 mg, 1.34 mmol, 1.5 equiv) and methyl (E)-4-[3-[4-(3-aminopropyl) piperazin-1-yl]propyl-methyl-amino]but-2-enoate (381 mg, 0.90 mmol, 1.0 equiv, TFA salt) in CH₂Cl₂ (6 mL) was added Et₃N(452 mg, 4.47 mmol, 622 mL, 5.0 equiv) at 0° C. The reaction mixture was warmed to 25° C. and stirred for 12 h. The mixture was concentrated in vacuo. The residue was purified by prep-HPLC (column: Phenomenex luna C18 150*40 mm* 15 μm; mobile phase: [water (0.225% FA)-ACN]; B %: 1%-30%, 10 min) to afford methyl (E)-4-[methyl-[3-[4-[3-[[4-[(2,2,2-trifluoroacetyl)amino]phenyl]sulfonylamino]propyl]piperazin-1-yl]propyl]amino]but-2-enoate (190 mg, 0.33 mmol, 38% yield) as a yellow oil. ¹HNMR (400 MHZ, DMSO-d₆): δ 7.92-7.87 (m, 2H), 7.85-7.80 (m, 2H), 7.65 (d, 1H, J=2.0 Hz), 6.89-6.78 (m, 1H), 6.10 (d, 1H, J=15.8 Hz), 3.67 (s, 3H), 3.36 (d, 2H, J=6.0 Hz), 2.93-2.60 (m, 13H), 2.58-2.53 (m, 3H), 2.31 (s, 3H), 1.77-1.69 (m, 2H), 1.63-1.55 (m, 2H).

### 18.3 Preparation of Compound 4

A mixture of methyl (E)-4-[methyl-[3-[4-[3-[[4-[(2,2,2-trifluoroacetyl)amino]phenyl]sulfonylamino]-propyl]piperazin-1-yl]propyl]amino]but-2-enoate (190 mg, 0.33 mmol, 1.0 equiv) in THF (4 mL) and 1 N aq. KOH (4 mL) was stirred at 25° C. for 12 h. The pH of the reaction mixture was adjusted to 5 by addition of 2 N aq. HCl. The reaction mixture was concentrated in vacuo to afford (E)-4-[3-[4-[3-[(4-aminophenyl)sulfonylamino]propyl]piperazin-1-yl]propyl-methyl-amino]but-2-enoic acid (150 mg, crude) as a yellow solid. ¹H NMR (400 MHZ, DMSO-d₆): δ 7.39 (d, 2H, J=8.8 Hz), 6.77-6.65 (m, 1H), 6.60 (d, 2H, J=8.8 Hz), 5.90 (d, 2H, J=15.6 Hz), 3.10 (d, 2H, J=5.6 Hz), 2.67 (t, 2H, J=6.8 Hz), 2.56-2.52 (m, 4H), 2.46-2.27 (m, 10H), 2.24-2.20 (m, 3H), 2.14 (s, 3H), 1.65-1.52 (m, 2H), 1.51-1.42 (m, 2H).

### 18.4 Preparation of Compound 5

A mixture of (E)-4-[3-[4-[3-[(4-aminophenyl)sulfonylamino]propyl]piperazin-1-yl]propyl-methyl-amino]but-2-enoic acid (120 mg, 0.26 mmol, 1.0 equiv), 2-[(5-bromo-2-chloro-pyrimidin-4-yl)amino]-6-fluoro-benzamide (91.4 mg, 0.26 mmol, 1.0 equiv) and 12 N aq. HCl (9.64 mg, 0.26 mmol, 1.0 equiv) in NMP (2 mL) was heated to 90° C. and stirred for 12 h. The mixture was purified by prep-HPLC (column: 3_Phenomenex Luna C18 75*30 mm*3 μm; mobile phase: [water (0.1% TFA)-ACN]; B %: 20%-40%,7 min) to afford (E)-4-[3-[4-[3-[[4-[5-bromo-4-(2-carbamoyl-3-fluoro-anilino) pyrimidin-2-yl]amino]phenyl]-sulfonylamino]propyl]piperazin-1-yl]propyl-methyl-amino]but-2-enoic acid (90.0 mg, 0.12 mmol, 45% yield) as a yellow oil. ¹H NMR (400 MHZ, DMSO-d₆): δ 10.14 (s, 1H), 9.96 (s, 1H), 8.40 (s, 1H), 8.28-8.22 (m, 1H), 8.17 (s, 1H), 8.10 (s, 1H), 7.87 (d, 2H, J=8.8 Hz), 7.64 (d, 2H, J=8.8 Hz), 7.58-7.49 (m, 2H), 7.12-7.08 (m, 1H), 6.85-6.71 (m, 1H), 6.22 (d, 1H, J=15.6 Hz), 3.95 (d, 2H, J=6.8 Hz), 3.20-2.89 (m, 9H), 2.81-2.73 (m, 6H), 2.59-2.54 (m, 4H), 1.93-1.85 (m, 2H), 1.81-1.73 (m, 2H).

### 18.5 Preparation of Compound I-18

A mixture of (E)-4-[3-[4-[3-[[4-[[5-bromo-4-(2-carbamoyl-3-fluoro-anilino) pyrimidin-2-yl]amino]phenyl]sulfonylamino]propyl]piperazin-1-yl]propyl-methyl-amino]but-2-enoic acid (45.0 mg, 0.06 mmol, 1.0 equiv) and 3-(4-phenoxyphenyl)-1-[(3R)-3-piperidyl]pyrazolo[3,4-d]pyrimidin-4-amine (29.3 mg, 0.06 mmol, 1.0 equiv, TFA salt), HOBt (12.0 mg, 0.09 mmol, 1.5 equiv), DIPEA (61.0 mg, 0.47 mmol, 82.2 mL, 8.0 equiv) and EDCI (17.0 mg, 0.09 mmol, 1.5 equiv) in DMF (0.5 mL) was stirred at 25° C. for 12 h. The mixture was purified by prep-HPLC (column: Unisil 3-100 C18 Ultra 150*50 mm*3 μm; mobile phase: [water (0.225% FA)-ACN]; B %: 15%-45%, 10 min) to afford 2-[[2-[4-[3-[4-[3-[[(E)-4-[(3R)-3-[4-amino-3-(4-phenoxyphenyl) pyrazolo[3,4-d]pyrimidin-1-yl]-1-piperidyl]-4-oxo-but-2-enyl]-methyl-amino]propyl]piperazin-1-yl]propylsulfamoyl]anilino]-5-bromo-pyrimidin-4-yl]amino]-6-fluoro-benzamide (23.3 mg, 0.02 mmol, 34% yield, 96% purity) as a white solid. ¹H NMR (400 MHZ, DMSO-d₆): δ 10.19-10.09 (m, 1H), 10.02-9.89 (m, 1H), 8.42-8.36 (m, 1H), 8.29-8.22 (m, 2H), 8.19-8.12 (m, 2H), 7.84 (d, 2H, J=8.4 Hz), 7.68-7.59 (m, 4H), 7.54-7.49 (m, 1H), 7.46-7.37 (m, 3H), 7.30-6.99 (m, 7H), 6.75-6.61 (m, 1H), 6.59-6.48 (m, 1H), 4.76-4.49 (m, 2H), 4.29-3.93 (m, 3H), 3.83-3.54 (m, 4H), 3.26-2.92 (m, 10H), 2.84-2.64 (m, 6H), 2.14-2.10 (m, 3H), 1.98-1.88 (m, 1H), 1.77-1.34 (m, 6H). LC-MS: MS (ES⁺): RT=2.581 min, m/z=1132.4 [M+H⁺]; LC-MS METHOD 01.

### Example 19—Synthesis of Compound I-19 (Synthesized from 5a below according to General Scheme D)

### General Scheme D

### Part I

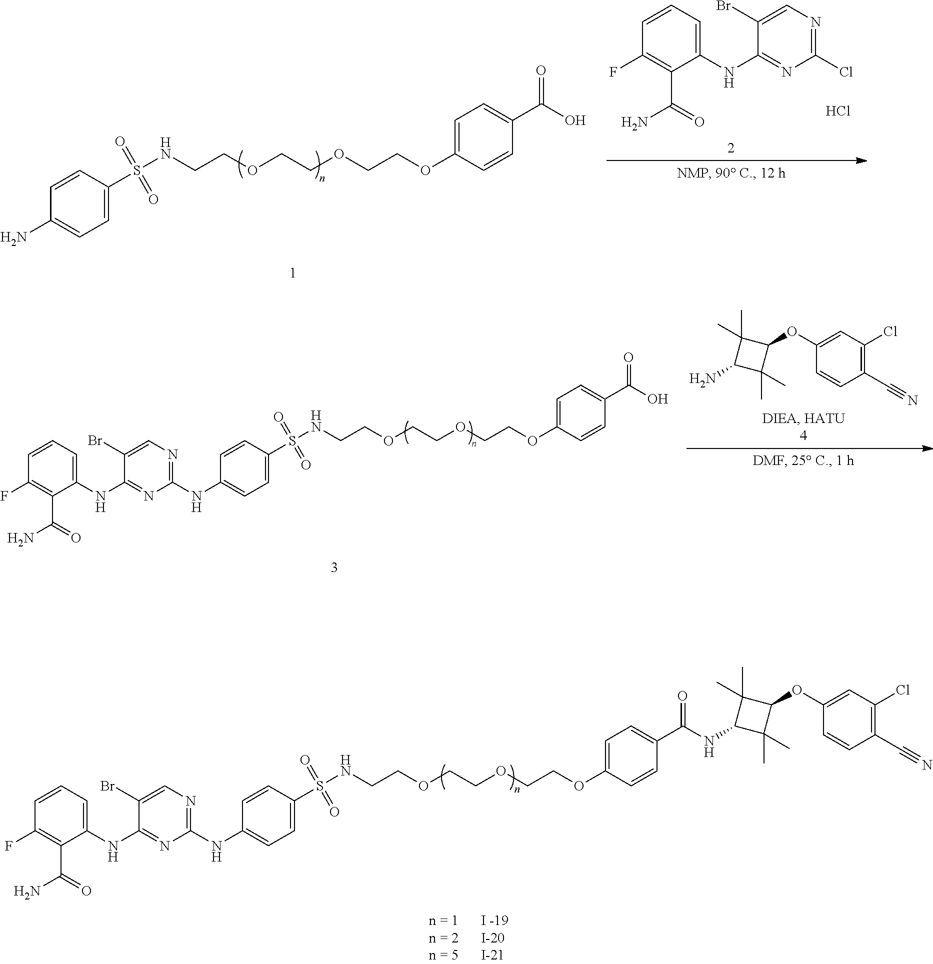

### Part II

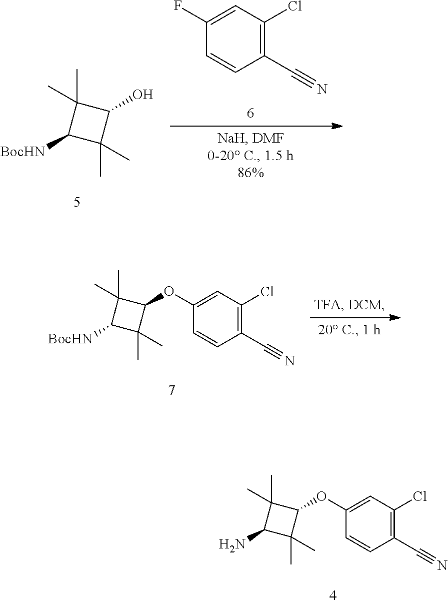 [SMILES: CC(C)(C)OC(=O)N[C@H]1C(C)(C)[C@H](O)C1(C)C ; N#Cc1ccc(F)cc1Cl ; CC(C)(C)OC(=O)N[C@H]1C(C)(C)[C@H](Oc2ccc(C#N)c(Cl)c2)C1(C)C ; CC1(C)[C@H](N)C(C)(C)[C@H]1Oc1ccc(C#N)c(Cl)c1]

### Synthesis of Compound I-19

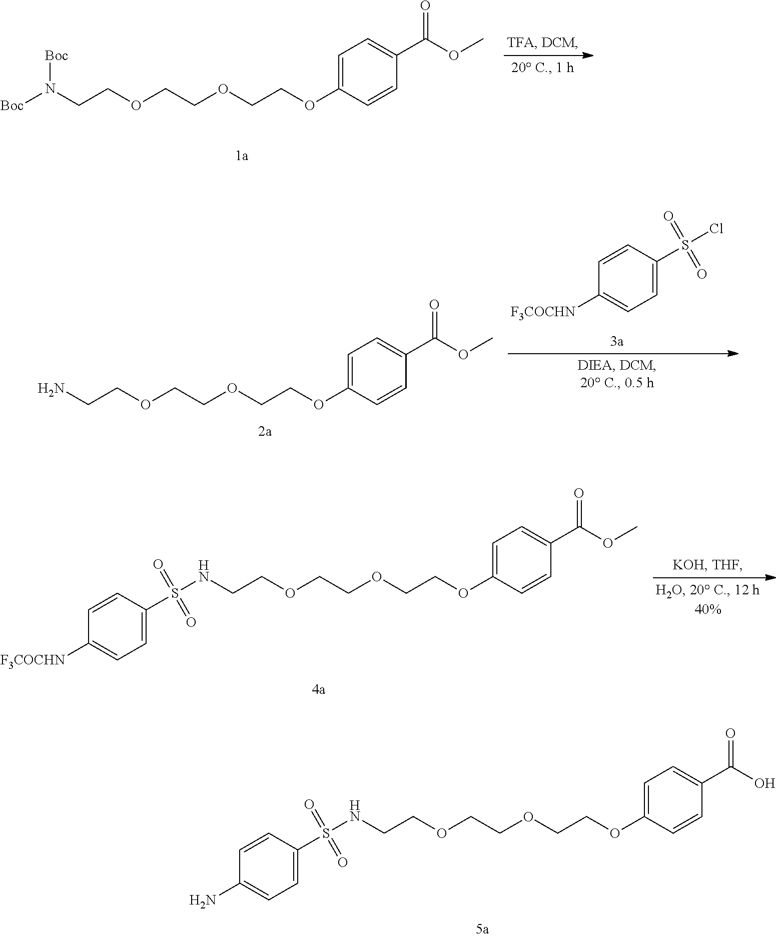

### 19.1 Preparation of Compound 2a

To a solution of compound 1a (500 mg, 1.0 mmol, 1.0 equiv) in DCM (5 mL) was added TFA (1 mL). The mixture was stirred at 20° C. for 1 h. The reaction mixture was concentrated under reduced pressure to give crude compound 2a (400 mg, 1.01 mmol, crude, TFA salt). The product was used directly for next step. LC-MS: MS (ES⁺): m/z=284.1 [M-55].

### 19.2 Preparation of Compound 4a

To a solution of compound 2a (400 mg, 1.0 mmol, TFA salt, 1.0 equiv) in DCM (10 mL) was added DIEA (638 mg, 4.94 mmol, 860 mL, 4.9 equiv) and compound 3 (347 mg, 1.21 mmol, 1.2 equiv). The mixture was stirred at 20° C. for 0.5 h. The reaction mixture was quenched by addition MeOH (2 mL), and then concentrated under reduced pressure to give a residue. The residue was purified by column chromatography (SiO₂, Petroleum ether/Ethyl acetate=1/1 to 1/3). Compound 4a (550 mg, crude) was obtained as a yellow oil. ¹H NMR (400 MHZ, CD₃OD): δ 7.95 (d, J=8.2 Hz, 2H), 7.86 (m, 4H), 7.00 (d, J=8.1 Hz, 2H), 4.00 (m, 2H), 3.91-3.80 (m, 5H), 3.78-3.62 (m, 4H), 3.54 (m, 2H), 3.47 (m, 2H), 3.27-3.18 (m, 2H), 3.06 (t, J=5.0 Hz, 2H).

### 19.3 Preparation of Compound 5a

To a solution of compound 4a (550 mg, 1.03 mmol, 1.0 equiv) in THF (10 mL) was added KOH (2 M, 2.5 mL, 4.8 equiv) in H₂O (2.5 mL). The mixture was stirred at 20° C. for 12 h. The mixture was adjusted with 2 N HCl to pH=5˜6. Then the mixture was concentrated under reduced pressure to give a residue. The residue was purified by prep-HPLC (column: 3_Phenomenex Luna C18 75*30 mm*3 μm; mobile phase: [water (0.05% HCl)-ACN]; B %: 24%-44%,6.5 min). Compound 5a (190 mg, 412 mmol, 40% yield, HCl salt) was obtained as a white solid. ¹H NMR (400 MHZ, CD₃OD): δ 8.06-7.87 (m, 4H), 7.49-7.36 (m, 2H), 7.01 (m, J=8.8 Hz, 2H), 4.21 (m, 2H), 3.86 (m, 2H), 3.70-3.62 (m, 2H), 3.54 (m, 2H), 3.48 (t, J=5.4 Hz, 2H), 3.06 (t, J=5.4 Hz, 2H). LC-MS: MS (ES⁺): m/z=447.1 [M+Na⁺].

### 19.4 Preparation of Compound 3 (General Scheme D)

To a solution of compound 1 (190 mg, 412 mmol, 1.0 equiv, HCl salt) and compound 2 (143 mg, 413 mmol, 1.0 equiv) in NMP (3 mL) was added HCl (12 M, 40 mL, 1.1 equiv). The mixture was stirred at 95° C. for 12 h. The reaction mixture was concentrated under reduced pressure to give a residue. The residue was purified by prep-HPLC (column: Phenomenex luna C18 150*40 mm* 15 μm; mobile phase: [water (0.1% TFA)-ACN]; B %: 29%-59%,11 min). Compound 7 (150 mg, 176 mmol, 42% yield, TFA salt) was obtained as a yellow solid. LC-MS: MS (ES⁺): m/z=734.9 [M+H⁺].

### 19.5 Preparation of Compound 7

To a mixture of compound 5 (2.0 g, 8.2 mmol, 1.0 equiv) in DMF (20 mL) was added NaH (493 mg, 12.3 mmol, 60% purity, 1.5 equiv) at 0° C. After addition, the mixture was stirred at 0° C. for 0.5 h. Then compound 6 (1.53 g, 9.86 mmol, 1.2 equiv) in DMF (5 mL) was added at 0° C. The mixture was stirred at 20° C. for 1 h. Saturated NH₄Cl (50 mL) solution was added at 0° C. The solution was extracted with EtOAc (3×50 mL). The combined organic layers were dried, filtered and concentrated to give a residue. The residue was purified by column chromatography (SiO₂, Petroleum ether/Ethyl acetate=10/1 to 5/1). Compound (2.7 g, 7.13 mmol, 86% yield) was obtained as a yellow solid. ¹HNMR (400 MHZ, CD₃OD): δ 7.69 (d, J=8.8 Hz, 1H), 7.08 (d, J=2.4 Hz, 1H), 6.94 (dd, J=2.4, 8.7 Hz, 1H), 4.14 (s, 1H), 3.65 (s, 1H), 1.45 (s, 9H), 1.22-1.09 (m, 12H). LC-MS: MS (ES⁺): m/z=323.1 [M+H⁺].

### 19.6 Preparation of Compound 4

To a solution of compound 7 (2.70 g, 7.13 mmol, 1.0 equiv) in DCM (20 mL) was added TFA (5 mL). The mixture was stirred at 20° C. for 1 h. The reaction mixture was concentrated under reduced pressure to give the desired product. Compound 4 (3.7 g, crude, TFA salt) was obtained as a yellow solid. LC-MS: MS (ES⁺): m/z=279.1 [M+H].

### 19.7 Preparation of Compound I-19

To a solution of compound 3 (50 mg, 59 mmol, 1.0 eq, TFA salt) and compound 4 (25 mg, 89 mmol, 1.5 equiv, TFA salt) in DMF (1 mL) was added DIEA (29 mg, 229 mmol, 40 mL, 3.9 equiv) and HATU (34 mg, 89 mmol, 1.5 equiv). The mixture was stirred at 20° C. for 0.5 h. The reaction mixture was quenched by addition H₂O (0.1 mL), and then filtered and concentrated under reduced pressure to give a residue. The residue was purified by prep-HPLC (column: Waters Xbridge 150*25 mm*5 μm; mobile phase: [water (10 mM NH₄HCO₃)-ACN]; B %: 54%-84%,9 min) to give desired compound 4-(2-(2-(2-(4-((5-bromo-4-((2-carbamoyl-3-fluorophenyl)amino)pyrimidin-2-yl)amino)phenylsulfonamido)ethoxy)ethoxy)ethoxy)-N-((1r,3r)-3-(3-chloro-4-cyanophenoxy)-2,2,4,4-tetramethylcyclobutyl) benzamide (26 mg, 26 mmol, 44% yield, 98% purity) as an off-white solid. ¹HNMR (400 MHZ, CD₃OD): δ 8.35 (d, J=8.5 Hz, 1H), 8.26 (s, 1H), 7.80 (m, 4H), 7.74-7.68 (m, 3H), 7.50 (m, 1H), 7.12 (d, J=2.4 Hz, 1H), 7.03-6.93 (m, 4H), 4.28 (s, 1H), 4.20-4.11 (m, 3H), 3.85-3.79 (m, 2H), 3.64 (m, 2H), 3.53 (m, 2H), 3.47 (t, J=5.4 Hz, 2H), 3.05 (t, J=5.4 Hz, 2H), 1.33-1.18 (m, 12H). LC-MS: MS (ES⁺): RT=2.499 min, m/z=995.3 [M+H⁺]; LC-MS METHOD 40.

### Example 20—Synthesis of Compound I-20 (Synthesized from 5a According to General Scheme D in a Similar Manner to Compound I-19)

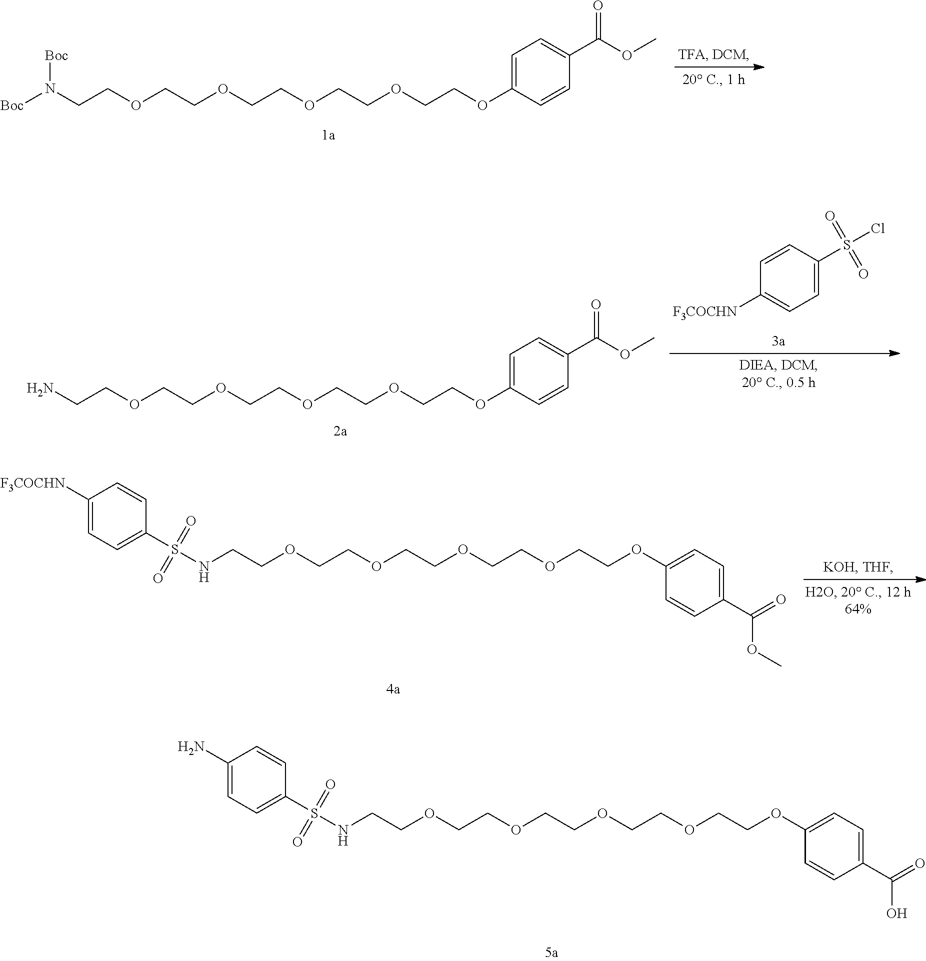

### 20.1 Preparation of 2a

To a solution of Compound 1 (1.00 g, 1.75 mmol, 1.0 equiv) in DCM (5 mL) was added TFA (1 mL). The mixture was stirred at 20° C. for 1 h. The reaction mixture was concentrated under reduced pressure to give crude compound 2 (0.84 g, 1.7 mmol, crude, TFA salt).

### 20.2 Preparation of 4a

To a solution of compound 2 (0.84 g, 1.7 mmol, 1.0 equiv, TFA salt) in DCM (15 mL) was added DIEA (1.1 g, 8.6 mmol, 1.5 mL, 4.9 equiv) and compound 3 (597 mg, 2.08 mmol, 1.2 equiv). The mixture was stirred at 20° C. for 0.5 h. The reaction mixture was quenched by addition MeOH (2 mL), and then concentrated under reduced pressure to give a residue. The residue was purified by column chromatography (SiO₂, Petroleum ether/Ethyl acetate=1/1 to 1/3). Compound 4 (600 mg, 963 mmol, crude) was obtained as a yellow oil.

### 20.3 Preparation of 5a

To a solution of compound 4 (600 mg, 963 mmol, 1.0 equiv) in THF (5 mL) was added KOH (2 M, 2.5 mL, 5.1 equiv) in H₂O (2.5 mL). The mixture was stirred at 20° C. for 12 h. The mixture was adjusted with 2 N HCl to pH=5˜6. Then the mixture was concentrated under reduced pressure to give a residue. The residue was purified by prep-HPLC (column: 3_Phenomenex Luna C18 75*30 mm*3 μm; mobile phase: [water (0.05% HCl)-ACN]; B %: 26%-46%,6.5 min). Compound 5 (340 mg, 619 mmol, 64% yield, HCl salt) was obtained as a yellow oil. ¹HNMR (400 MHZ, CD₃OD): δ 8.05-7.93 (m, 4H), 7.58-7.51 (m, 2H), 7.03-6.97 (m, 2H), 4.21 (m, 2H), 3.87 (m, 2H), 3.75-3.70 (m, 2H), 3.69-3.63 (m, 4H), 3.63-3.59 (m, 2H), 3.59-3.53 (m, 2H), 3.51-3.41 (m, 4H), 3.08 (t, J=5.4 Hz, 2H). LC-MS: MS (ES⁺): m/z=514.3 [M+H⁺].

### 20.5 Preparation of Compound I-20

4-((14-(4-((5-bromo-4-((2-carbamoyl-3-fluorophenyl)amino)pyrimidin-2-yl)amino)phenyl-sulfonamido)-3,6,9,12-tetraoxatetradecyl)oxy)-N-((1r,3r)-3-(3-chloro-4-cyanophenoxy)-2,2,4,4-tetramethylcyclobutyl) benzamide (40 mg, 36 mmol, 67% yield, 97% purity) as an off-white solid. ¹HNMR (400 MHZ, CD₃OD): δ 8.35 (d, J=8.5 Hz, 1H), 8.26 (s, 1H), 7.84 (m, 2H), 7.77 (m, 2H), 7.75-7.68 (m, 3H), 7.51 (m, 1H), 7.12 (d, J=2.4 Hz, 1H), 7.02-6.94 (m, 4H), 4.27 (s, 1H), 4.19-4.11 (m, 3H), 3.86-3.80 (m, 2H), 3.71-3.67 (m, 2H), 3.66-3.57 (m, 6H), 3.57-3.53 (m, 2H), 3.50-3.41 (m, 4H), 3.04 (t, J=5.4 Hz, 2H), 1.29-1.19 (m, 12H). LC-MS: MS (ES⁺): RT=2.497 min, m/z=1083.5 [M+H⁺]; LC-MS METHOD 40.

### Example 21—Synthesis of Compound I-21 (Synthesized from 5a Below According to General Scheme D in a Similar Manner to Compound I-19)

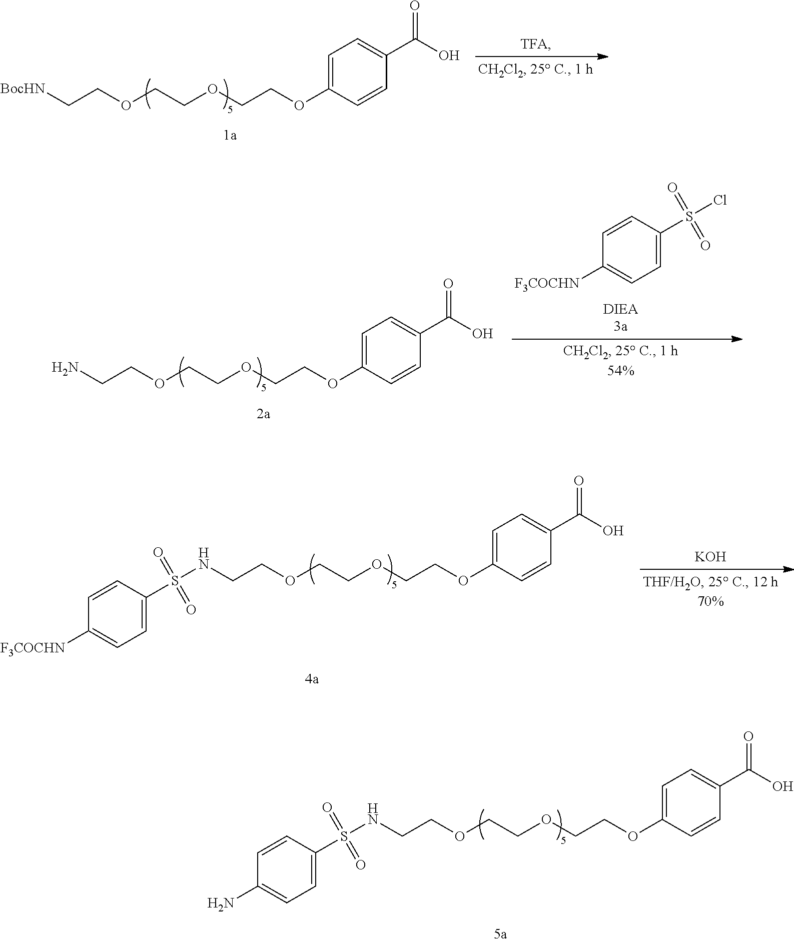

### 21.1 Preparation of 2a

To a solution of 4-[2-[2-[2-[2-[2-[2-[2-(tert-butoxycarbonylamino)ethoxy]ethoxy]ethoxy]ethoxy]ethoxy]ethoxy]ethoxy]benzoic acid (300 mg, 550 mmol, 1.0 equiv) in CH₂Cl₂ (2 mL) was added TFA (1 mL), and then it was stirred at 25° C. for 1 h. The reaction mixture was concentrated to 4-[2-[2-[2-[2-[2-[2-(2-aminoethoxy)ethoxy]ethoxy]ethoxy]ethoxy]ethoxy]ethoxy]benzoic acid (300 mg, 536 mmol, 97% yield, TFA salt) was obtained as a colorless oil and used for the next step directly.

### 21.2 Preparation of 4a

To a solution of 4-[2-[2-[2-[2-[2-[2-(2-aminoethoxy)ethoxy]ethoxy]ethoxy]ethoxy]ethoxy]ethoxy]benzoic acid (300 mg, 536 mmol, 1.0 equiv, TFA salt) and DIEA (346 mg, 2.68 mmol, 467 mL, 5.0 equiv) in CH₂Cl₂ (2 mL) was added 4-[(2,2,2-trifluoro-2-oxo-ethyl)amino]benzenesulfonyl chloride (233 mg, 804 mmol, 1.5 equiv), and then it was stirred at 25° C. for 1 h. pH was adjusted to 6-7 by TFA and then it was concentrated to afford crude product. The residue was purified by prep-HPLC (column: Phenomenex luna C18 150*40 mm*15 μm; mobile phase: [water (0.1% TFA)-ACN]; B %: 45%-75%, 11 min) to afford 4-[2-[2-[2-[2-[2-[2-[2-[[4-[(2,2,2-trifluoroacetyl)amino]phenyl]sulfonylamino]ethoxy]ethoxy]ethoxy]ethoxy]ethoxy]ethoxy]ethoxy]benzoic acid (200 mg, 287 mmol, 54% yield) as a white solid. ¹H NMR: (400 MHz, CD₃OD) δ 8.11-8.04 (m, 2H), 7.87 (s, 4H), 7.15-7.05 (m, 2H), 4.25 (dd, J=3.7, 5.4 Hz, 2H), 3.88 (dd, J=5.3 Hz, 2H), 3.73-3.69 (m, 2H), 3.68-3.56 (m, 14H), 3.56-3.51 (m, 2H), 3.50-3.40 (m, 4H), 3.05 (t, J=5.4 Hz, 2H).

### 21.3 Preparation of 5a

To a solution of 4-[2-[2-[2-[2-[2-[2-[2-[4-[(2,2,2-trifluoroacetyl)amino]phenyl]sulfonylamino]-ethoxy]ethoxy]ethoxy]ethoxy]ethoxy]ethoxy]ethoxy]benzoic acid (200 mg, 287 mmol, 1.0 equiv) in THF (2 mL) was added KOH (161 mg, 2.87 mmol, 10.0 equiv) in H₂O (1 mL), and then it was stirred at 25° C. for 12 hr. pH was adjusted to 6-7 by con. HCl at 0° C., and then it was concentrated to afford crude product. The residue was purified by prep-HPLC (column: 3_Phenomenex Luna C18 75*30 mm*3 μm; mobile phase: [water (0.05% HCl)-ACN]; B %: 28%-48%, 6.5 min) to afford 4-[2-[2-[2-[2-[2-[2-[2-[(4-aminophenyl)-sulfonylamino]ethoxy]ethoxy]ethoxy]ethoxy]ethoxy]ethoxy]ethoxy]benzoic acid (120 mg, 200 mmol, 70% yield) as a colorless solid. LC-MS: MS (ES⁺): m/z=601.1 [M+H⁺].

### 21.4 Preparation of Compound I-21

2-[[5-bromo-2-[4-[2-[2-[2-[2-[2-[2-[2-[4-[[3-(3-chloro-4-cyano-phenoxy)-2,2,4,4-tetramethylcyclobutyl]carbamoyl]phenoxy]ethoxy]ethoxy]ethoxy]ethoxy]ethoxy]ethoxy]ethylsulfamoyl]anilino]pyrimidin-4-yl]amino]-6-fluoro-benzamide (33 mg, 28 mmol, 43% yield) as a white solid. LC-MS: MS (ES⁺): RT=2.537 min, m/z=1169.2, 1171.2 [M+H⁺], LC-MS METHOD 40. ¹H NMR: (400 MHz, CD₃OD) δ 8.35 (d, J=8.5 Hz, 1H), 8.27 (s, 1H), 7.88-7.81 (m, 2H), 7.81-7.75 (m, 2H), 7.71 (d, J=8.8 Hz, 3H), 7.50 (m, 1H), 7.12 (d, J=2.4 Hz, 1H), 7.03-6.94 (m, 4H), 4.27 (s, 1H), 4.18-4.11 (m, 3H), 3.83 (m, 2H), 3.70-3.51 (m, 18H), 3.50-3.42 (m, 4H), 3.04 (t, J=5.4 Hz, 2H), 1.27 (s, 6H), 1.22 (s, 6H).

### Example 22—Synthesis of Compound I-22 (Synthesized According to General Scheme E)

### General Scheme E

### Part I

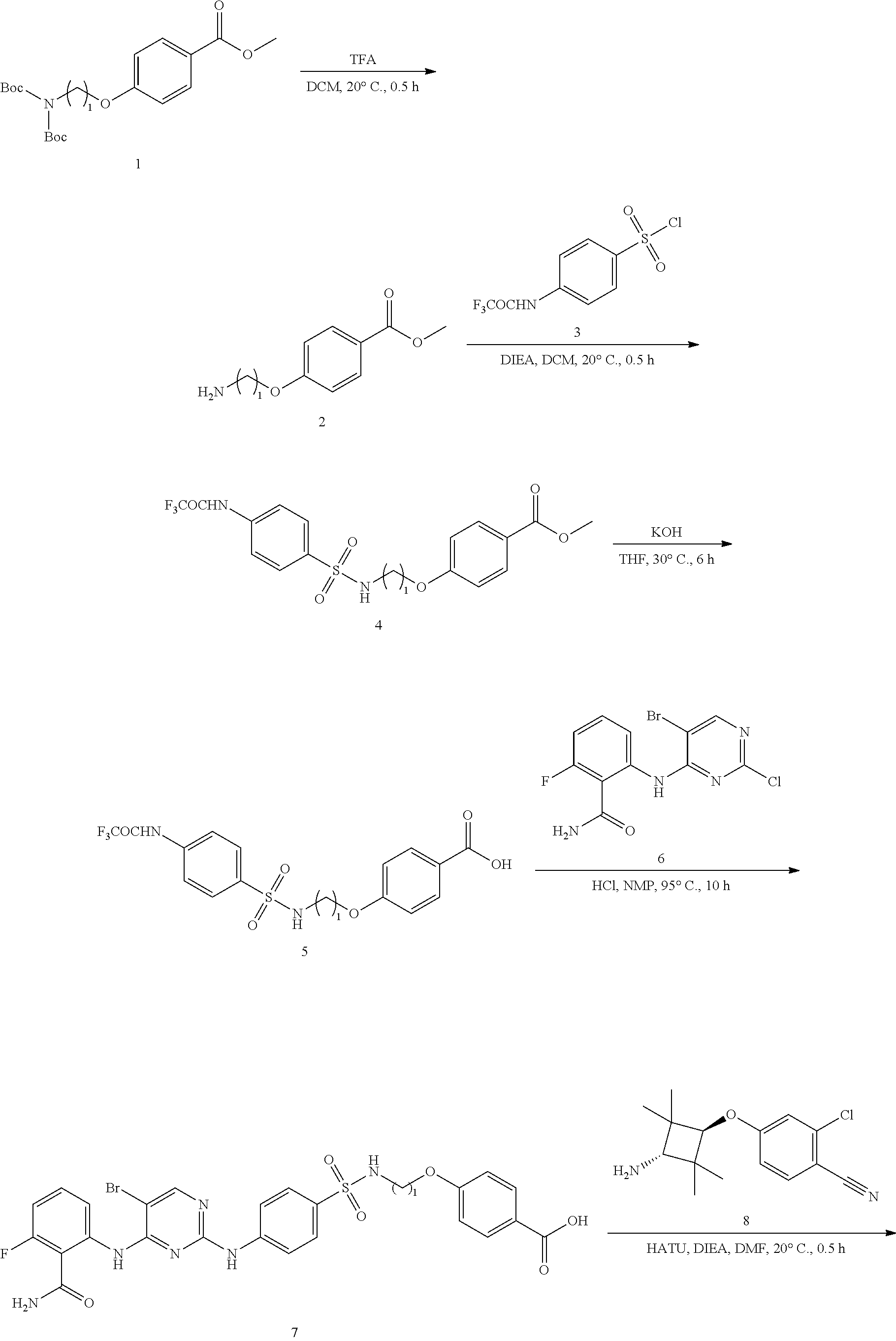

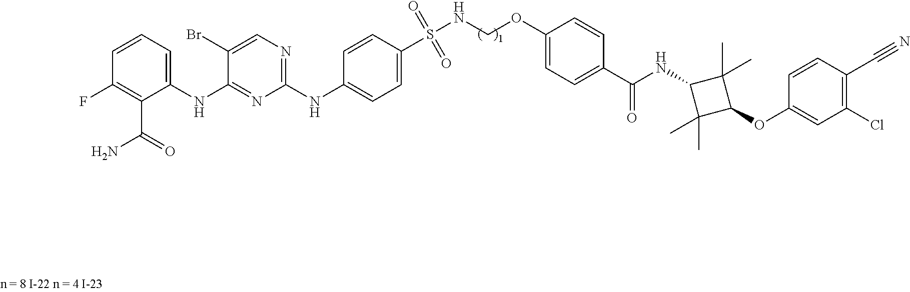

### Part II

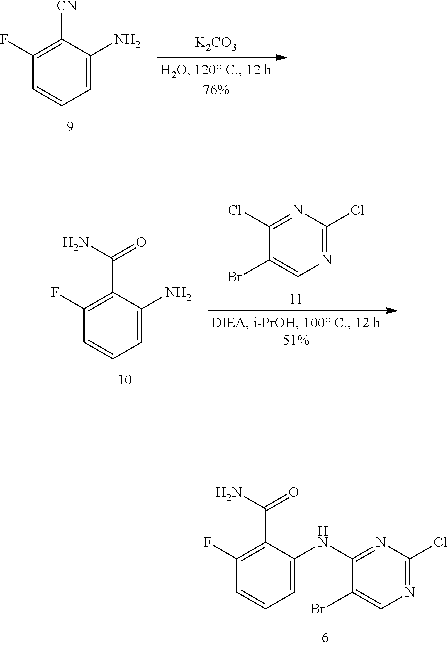

### Synthesis of Compound I-22

### 22.1 Preparation of Compound 2

To a solution of compound 1 (550 mg, 1.15 mmol, 1.0 equiv) in CH₂Cl₂ (8 mL) was added TFA (2 mL). The mixture was stirred at 20° C. for 0.5 h. The reaction mixture was concentrated under reduced pressure to give crude compound 2 (450 mg, 1.14 mmol, crude, TFA salt). The crude product was used directly for next step. LC-MS: MS (ES⁺): m/z=280.1 [M+H⁺].

### 22.2 Preparation of Compound 4

To a solution of compound 2 (450 mg, 1.14 mmol, TFA salt, 1.0 equiv) in DCM (5 mL) was added DIEA (739 mg, 5.72 mmol, 996 mL, 5.0 equiv) and compound 3 (394 mg, 1.37 mmol, 1.2 equiv). The mixture was stirred at 20° C. for 0.5 h. The reaction mixture was concentrated under reduced pressure to give a residue. The residue was purified by column chromatography (SiO₂, Petroleum ether/Ethyl acetate=3/1 to 1/1). Compound 4 (606 mg, 1.14 mmol, 99% yield) was obtained as a white solid. ¹HNMR (400 MHZ, DMSO-d₆): δ=11.59 (s, 1H), 7.92-7.85 (m, 4H), 7.84-7.79 (m, 2H), 7.59-7.53 (m, 1H), 7.01 (d, J=8.9 Hz, 2H), 4.04-4.00 (m, 2H), 3.80 (s, 3H), 2.77-2.68 (m, 2H), 1.76-1.60 (m, 2H), 1.35 (m, 4H), 1.27-1.16 (m, 9H). LC-MS: MS (ES⁺): m/z=553.2 [M+Na⁺].

### 22.3 Preparation of Compound 5

To a solution of compound 4 (606 mg, 1.14 mmol, 1.0 equiv) in THF (5 mL) was added KOH (320 mg, 5.71 mmol, 5.0 equiv) in H₂O (4 mL). The mixture was stirred at 30° C. for 6 h. The mixture was adjusted with 2 N HCl to pH=5˜6. Then the mixture was concentrated under reduced pressure to give a residue. The residue was purified by prep-HPLC (column: 3_Phenomenex Luna C18 75*30 mm*3 μm; mobile phase: [water (0.05% HCl)-ACN]; B %: 43%-63%,6.5 min). Compound 5 (160 mg, 380 mmol, 33% yield, HCl salt) was obtained as a white solid. LC-MS: MS (ES⁺): m/z=443.3 [M+Na⁺].

### 22.4 Preparation of Compound 10

To a solution of compound 9 (10 g, 73 mmol, 1.0 equiv) in H₂O (500 mL) was added K₂CO₃ (2 g, 15 mmol, 0.2 equiv). The mixture was stirred at 120° C. for 12 h. The reaction mixture was extracted with EtOAc (3×500 mL). The combined organic layers were dried over Na₂SO₄, filtered and concentrated under reduced pressure to give a residue. The residue was purified by column chromatography (SiO₂, Petroleum ether/Ethyl acetate=10/1 to 3/1). Compound 7 (9 g, 56 mmol, 76% yield) was obtained as a white solid. ¹HNMR (400 MHZ, DMSO-d₆): δ 7.52 (m, 2H), 7.13-7.00 (m, 1H), 6.51 (d, J=8.3 Hz, 1H), 6.35-6.26 (m, 1H), 6.17 (s, 2H).

### 22.5 Preparation of Compound 6

To a solution of compound 10 (8.7 g, 56 mmol, 1.0 equiv) and compound 6 (12.8 g, 56 mmol, 7.2 mL, 1.0 equiv) in i-PrOH (40 mL) was added DIEA (8.7 g, 67 mmol, 11.8 mL, 1.2 equiv). The mixture was stirred at 100° C. for 12 h. The reaction mixture was filtered and the filter cake was concentrated under reduced pressure to give compound 6 (10 g, 29 mmol, 51% yield) as a yellow solid. ¹HNMR (400 MHZ, DMSO-d₆): δ 10.64 (s, 1H), 8.57 (s, 1H), 8.25-8.06 (m, 3H), 7.62-7.52 (m, 1H), 7.17-7.08 (m, 1H). LC-MS: MS (ES⁺): m/z=347.0 [M+H⁺].

### 22.6 Preparation of Compound 7

To a solution of compound 5 (160 mg, 380 mmol, 1.0 equiv, HCl salt) and compound 6 (131 mg, 380 mmol, 1.0 equiv) in NMP (2 mL) was added HCl (12 M, 35 mL, 1.1 equiv). The mixture was stirred at 95° C. for 10 h. The reaction mixture was concentrated under reduced pressure to give a residue. The residue was purified by prep-HPLC (column: 3_Phenomenex Luna C18 75*30 mm*3 μm; mobile phase: [water (0.1% TFA)-ACN]; B %: 51%-81%,7 min). Compound 7 (120 mg, 142 mmol, 37% yield, TFA salt) was obtained as a white solid. LC-MS: MS (ES⁺): m/z=731.1 [M+H⁺].

### 22.7 Preparation of Compound I-22

To a solution of compound 7 (120 mg, 142 mmol, 1.0 equiv, TFA salt) and compound 8 (84 mg, 213 mmol, 1.5 equiv, TFA salt) in DMF (1 mL) was added DIEA (55 mg, 426 mmol, 74 mL, 3.0 equiv) and HATU (81 mg, 213 mmol, 1.5 equiv). The mixture was stirred at 20° C. for 0.5 h. The reaction mixture was quenched by addition H₂O (0.1 mL), and concentrated under reduced pressure to give a residue. The residue was purified by prep-HPLC (column: Waters Xbridge 150*25 mm*5 μm; mobile phase: [water (10 mM NH₄HCO₃)-ACN]; B %: 66%-96%,9 min) to give desired compound 4-((8-(4-((5-bromo-4-((2-carbamoyl-3-fluorophenyl)amino)pyrimidin-2-yl)amino)phenylsulfonamido)octyl)oxy)-N-((1r,3r)-3-(3-chloro-4-cyanophenoxy)-2,2,4,4-tetramethylcyclobutyl) benzamide (106 mg, 107 mmol, 75% yield, 99% purity) as an off-white solid. ¹HNMR (400 MHZ, CD₃OD): δ 8.36 (d, J=8.6 Hz, 1H), 8.25 (s, 1H), 7.84 (d, J=8.8 Hz, 2H), 7.76 (d, J=8.8 Hz, 2H), 7.74-7.68 (m, 3H), 7.53-7.46 (m, 1H), 7.13 (d, J=2.3 Hz, 1H), 7.02-6.95 (m, 2H), 6.92 (d, J=8.8 Hz, 2H), 4.28 (s, 1H), 4.13 (s, 1H), 3.99-3.88 (m, 2H), 2.90-2.82 (m, 2H), 1.74-1.66 (m, 2H), 1.46-1.34 (m, 4H), 1.33-1.16 (m, 18H). LC-MS: MS (ES⁺): RT=2.971 min, m/z=991.3 [M+H⁺]; LC-MS METHOD 40.

### Example 23—Synthesis of Compound I-23 (synthesized according to General Scheme E in a similar manner to Compound I-23)

4-(4-(4-((5-bromo-4-((2-carbamoyl-3-fluorophenyl)amino)pyrimidin-2-yl)amino)phenylsulfonamido) butoxy)-N-((1r,3r)-3-(3-chloro-4-cyanophenoxy)-2,2,4,4-tetramethylcyclobutyl) benzamide (47 mg, 50 mmol, 61% yield, 99% purity) as an off-white solid. ¹H NMR (400 MHZ, CD₃OD): δ 8.34 (d, J=7.9 Hz, 1H), 8.27 (s, 1H), 7.82 (d, J=8.7 Hz, 2H), 7.76-7.67 (m, 5H), 7.54-7.45 (m, 1H), 7.13 (s, 1H), 7.02-6.94 (m, 2H), 6.91 (d, J=8.7 Hz, 2H), 4.27 (s, 1H), 4.12 (s, 1H), 4.00-3.91 (m, 2H), 2.99-2.90 (m, 2H), 1.85-1.73 (m, 2H), 1.68-1.56 (m, 2H), 1.27 (s, 6H), 1.22 (s, 6H). LC-MS: MS (ES⁺): RT=3.156 min, m/z=935.0 [M+H⁺]; LC-MS METHOD 10.

### Example 24—Synthesis of Compound I-24

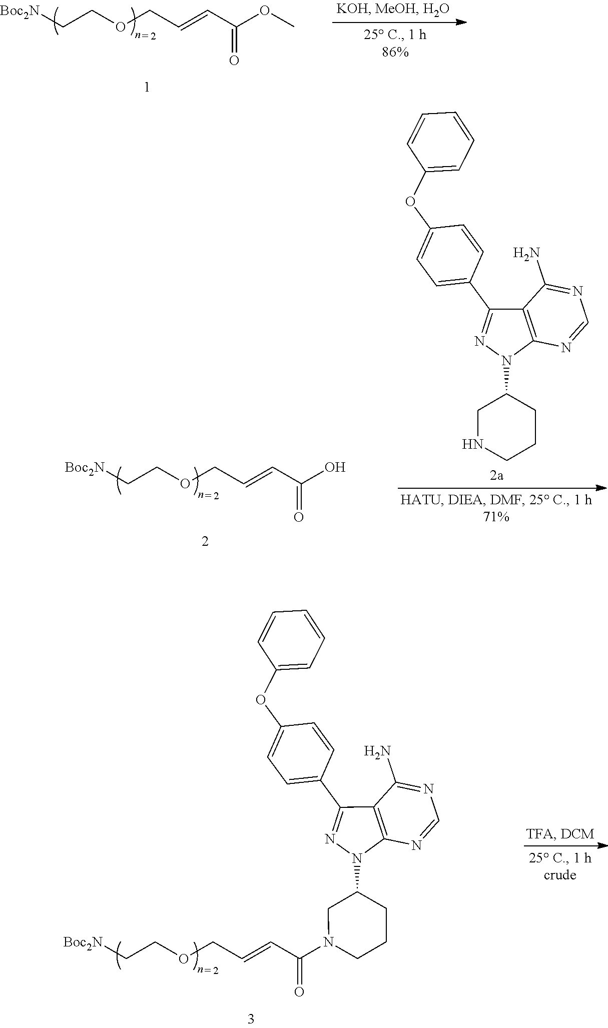

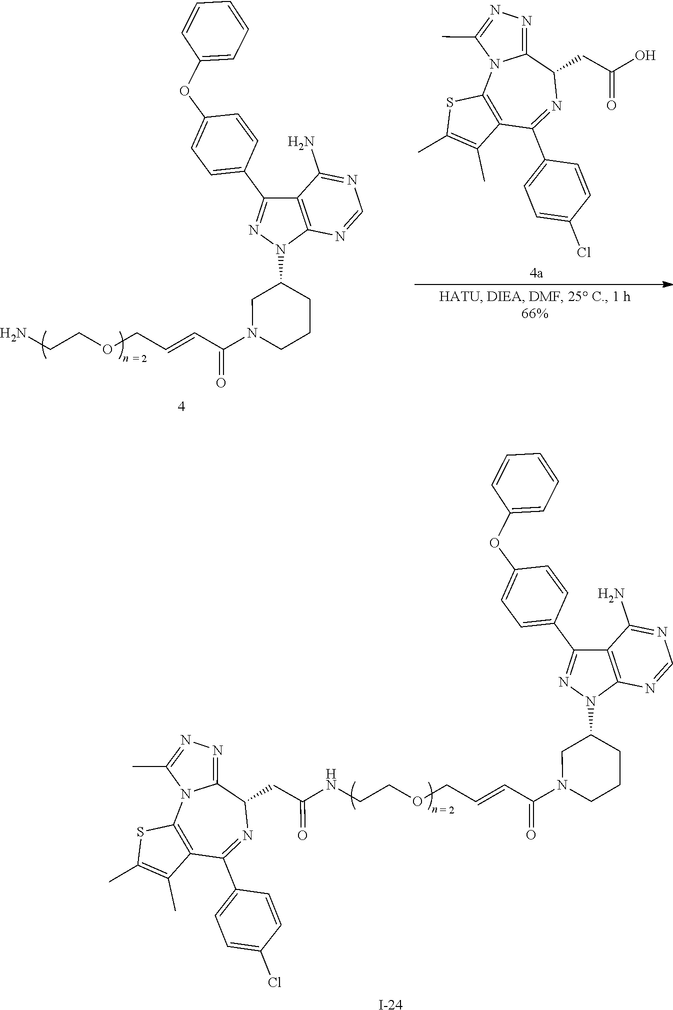

### 24.1 Preparation of Compound 2

To a solution of methyl (E)-4-[2-[2-[bis(tert-butoxycarbonyl)amino]ethoxy]ethoxy]but-2-enoate (0.60 g, 1.5 mmol, 1.0 equiv) in MeOH (6 mL) and H₂O (2 mL) was added KOH (0.13 g, 2.23 mmol, 1.5 equiv). The mixture was stirred at 25° C. for 1 h. The reaction mixture was concentrated under reduced pressure to give crude (E)-4-[2-[2-[bis(tert-butoxycarbonyl)amino]ethoxy]ethoxy]but-2-enoic acid (0.50 g, 1.3 mmol, 86% yield) as a yellow oil. LC-MS: MS (ES⁺): RT=0.834 min, m/z=190.5 [M+H⁺-200].

### 24.2 Preparation of Compound 3

To a solution of (E)-4-[2-[2-[bis(tert-butoxycarbonyl)amino]ethoxy]ethoxy]but-2-enoic acid (1.0 equiv) and (R)-3-(4-phenoxyphenyl)-1-(piperidin-3-yl)-1H-pyrazolo[3,4-d]pyrimidin-4-amine (1.0 equiv) in DMF were added DIEA (4.0 equiv) and HATU (1.2 equiv). The mixture was stirred at about 25° C. for about 1 hour. Then, the solution was purified by prep-HPLC (column: Waters Xbridge C18 150*50 mm*10 μm; mobile phase: [water (10 mM NH₄HCO₃)-ACN]; B %: 55%-85%, 10 min) to give tert-butyl-N-[2-[2-[(E)-4-[(3R)-3-[4-amino-3-(4-phenoxyphenyl) pyrazolo[3,4-d]pyrimidin-1-yl]-1-piperidyl]-4-oxo-but-2-enoxy]ethoxy]ethyl]-N-tert-butoxycarbonyl-carbamate (0.55 g, 0.73 mmol, 71% yield) as a yellow oil. LC-MS: MS (ES⁺): RT=0.886 min, m/z=758.2 [M+H⁺].

### 24.3 Preparation of Compound 4

A mixture of tert-butyl-N-[2-[2-[(E)-4-[(3R)-3-[4-amino-3-(4-phenoxyphenyl) pyrazolo[3,4-d]pyrimidin-1-yl]-1-piperidyl]-4-oxo-but-2-enoxy]ethoxy]ethyl]-N-tert-butoxycarbonyl-carbamate (1.0 equiv) in DCM and TFA was stirred at about 25° C. for about 1 hour. The mixture was concentrated to give crude (E)-4-[2-(2-aminoethoxy)ethoxy]-1-[(3R)-3-[4-amino-3-(4-phenoxyphenyl) pyrazolo[3,4-d]pyrimidin-1-yl]-1-piperidyl]but-2-en-1-one (0.09 g, 0.13 mmol, TFA salt) as a yellow oil.

### 24.4 Preparation of Compound I-24

To a solution of (E)-4-[2-(2-aminoethoxy)ethoxy]-1-[(3R)-3-[4-amino-3-(4-phenoxyphenyl) pyrazolo[3,4-d]pyrimidin-1-yl]-1-piperidyl]but-2-en-1-one (84 mg, 0.13 mmol, 1.0 equiv, TFA salt) in DMF (2 mL) were added HATU (62 mg, 0.16 mmol, 1.3 equiv), 2-[(9S)-7-(4-chlorophenyl)-4,5,13-trimethyl-3-thia-1,8,11,12-tetrazatricyclo[8.3.0.02.6]trideca-2 (6),4,7,10,12-pentaen-9-yl]acetic acid (50 mg, 0.13 mmol, 1.0 equiv) and DIEA (48 mg, 0.37 mmol, 3.0 equiv). The mixture was stirred at 25° C. for 1 h. The solution was purified by prep-HPLC (column: Waters Xbridge 150*25 mm*5 μm; mobile phase: [water (10 mM NH₄HCO₃)-ACN]; B %: 45%-75%, 9 min) to give N-[2-[2-[(E)-4-[(3R)-3-[4-amino-3-(4-phenoxyphenyl) pyrazolo[3,4-d]pyrimidin-1-yl]-1-piperidyl]-4-oxo-but-2-enoxy]ethoxy]ethyl]-2-[(9S)-7-(4-chlorophenyl)-4,5,13-trimethyl-3-thia-1,8,11,12-tetrazatricyclo[8.3.0.02.6]trideca-2 (6),4,7,10,12-pentaen-9-yl]acetamide (77 mg, 82 mmol, 66% yield, 100% purity) as a white solid. ¹H NMR (400 MHZ, METHANOL-d₄): δ 8.25-8.17 (m, 1H), 7.65 (t, J=8.0 Hz, 2H), 7.49-7.30 (m, 6H), 7.22-7.02 (m, 5H), 6.83-6.66 (m, 1H), 6.66-6.44 (m, 1H), 4.74-4.49 (m, 2H), 4.28-3.75 (m, 4H), 3.73-3.36 (m, 12H), 2.72-2.65 (m, 3H), 2.43 (s, 3H), 2.31-1.91 (m, 3H), 1.75-1.58 (m, 4H). LC-MS: MS (ES⁺): RT=2.370 min, m/z=940.2 [M+H⁺]; LC-MS method 25.

### Example 25—Synthesis of Compound I-25

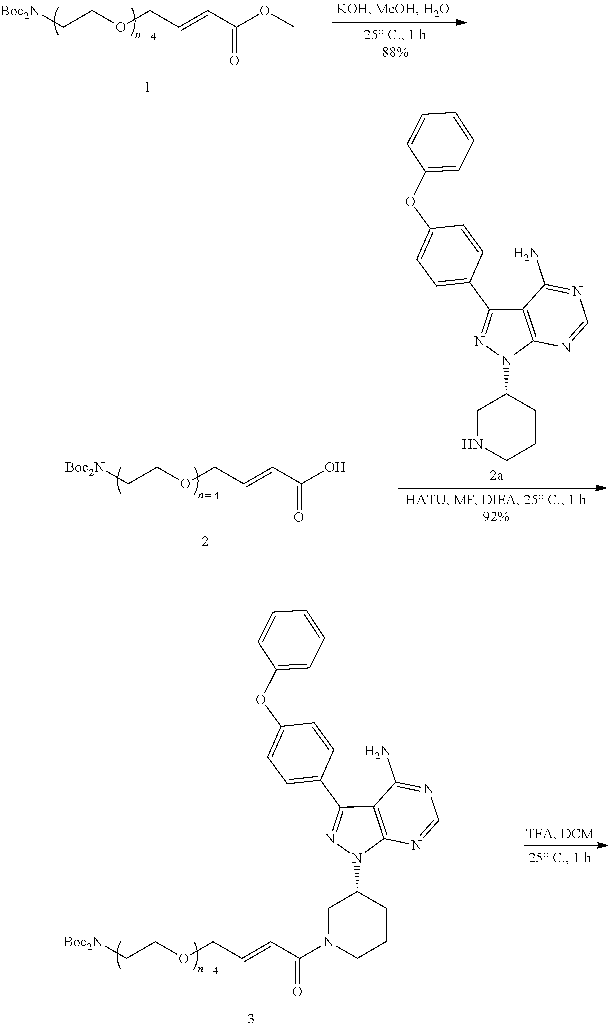

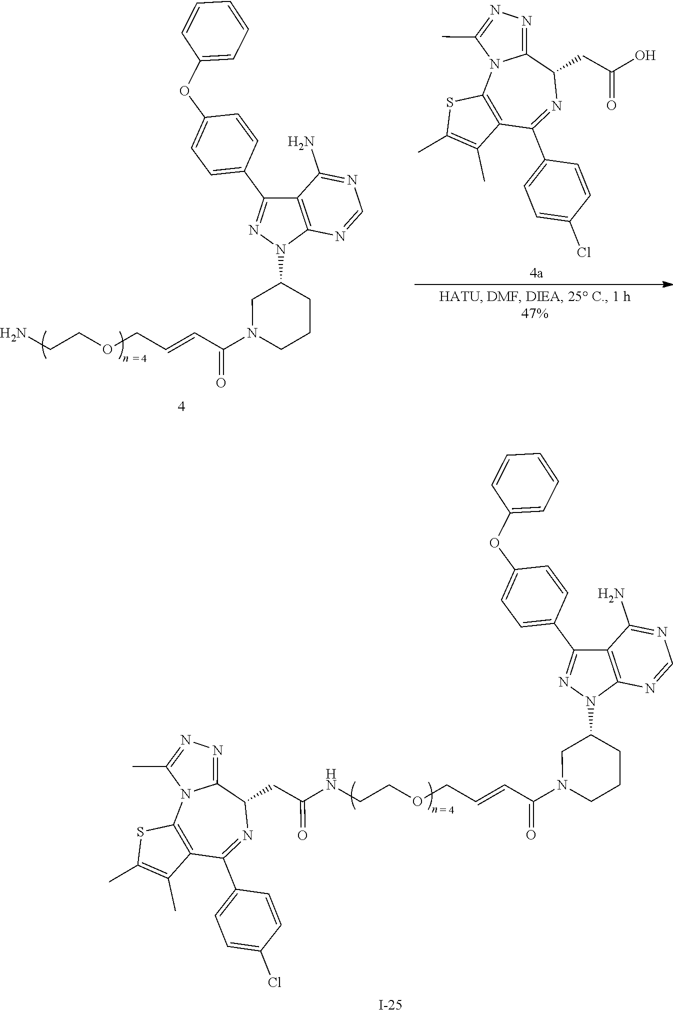

### 25.1 Preparation of Compound 2

Prepared similarly to compound 2 in Compound I-24. The reaction mixture was concentrated to give (E)-4-[2-[2-[2-[2-[bis(tert-butoxycarbonyl)amino]ethoxy]ethoxy]ethoxy]ethoxy]but-2-enoic acid (0.60 g, 1.3 mmol, 88% yield) as a yellow oil. LC-MS: MS (ES⁺): RT=0.668 min, m/z=278.2 [M+H⁺-200].

### 25.2 Preparation of Compound 3

Prepared similarly to compound 3 in Compound I-24. The product was purified by prep-HPLC (column: Waters Xbridge C18 150*50 mm*10 μm; mobile phase: [water (10 mM NH₄HCO₃)-ACN]; B %: 47%-77%, 10 min) to give tert-butyl-N-[2-[2-[2-[2-[(E)-4-[(3R)-3-[4-amino-3-(4-phenoxyphenyl) pyrazolo[3,4-d]pyrimidin-1-yl]-1-piperidyl]-4-oxo-but-2-enoxy]ethoxy]ethoxy]ethoxy]ethyl]-N-tert-butoxycarbonyl-carbamate (0.80 g, 0.95 mmol, 92% yield) as a yellow oil. LC-MS: MS (ES⁺): RT=0.750 min, m/z=846.3 [M+H⁺].

### 25.3 Preparation of Compound 4

Prepared similarly to compound 4 in Compound I-24. The mixture was concentrated to give crude (E)-4-[2-[2-[2-(2-aminoethoxy)ethoxy]ethoxy]ethoxy]-1-[(3R)-3-[4-amino-3-(4-phenoxyphenyl) pyrazolo[3,4-d]pyrimidin-1-yl]-1-piperidyl]but-2-en-1-one (0.10 g, 0.13 mmol, TFA salt) as a yellow oil.

### 25.4 Preparation of Compound I-25

To a solution of (E)-4-[2-[2-[2-(2-aminoethoxy)ethoxy]ethoxy]ethoxy]-1-[(3R)-3-[4-amino-3-(4-phenoxyphenyl) pyrazolo[3,4-d]pyrimidin-1-yl]-1-piperidyl]but-2-en-1-one (65 mg, 0.12 mmol, 1.0 equiv, TFA salt) in DMF (2 mL) were added HATU (71 mg, 0.19 mmol, 1.5 equiv), 2-[(9S)-7-(4-chlorophenyl)-4,5,13-trimethyl-3-thia-1,8,11,12-tetrazatricyclo[8.3.0.02.6]trideca-2 (6),4, 7, 10, 12-pentaen-9-yl]acetic acid (50 mg, 0.12 mmol, 1.0 equiv) and DIEA (48 mg, 0.37 mmol, 3.0 equiv). The mixture was stirred at 25° C. for 1 h. The solution was purified by prep-HPLC (column: Waters Xbridge 150*25 mm*5 μm; mobile phase: [water (10 mM NH₄HCO₃)-ACN]; B %: 45%-75%, 8 min) to give N-[2-[2-[2-[2-[(E)-4-[(3R)-3-[4-amino-3-(4-phenoxyphenyl) pyrazolo[3,4-d]pyrimidin-1-yl]-1-piperidyl]-4-oxo-but-2-enoxy]ethoxy]ethoxy]ethoxy]ethyl]-2-[(9S)-7-(4-chlorophenyl)-4,5,13-trimethyl-3-thia-1,8,11,12-tetrazatricyclo[8.3.0.0^{2,6}]trideca-2 (6),4,7, 10, 12-pentaen-9-yl]acetamide (60 mg, 58 mmol, 47% yield, 100% purity) as a white solid. ¹H NMR (400 MHZ, METHANOL-d₄): δ 8.28-8.18 (m, 1H), 7.66 (d, J=8.4 Hz, 2H), 7.48-7.32 (m, 6H), 7.20-7.05 (m, 5H), 6.73 (s, 1H), 6.64-6.40 (m, 1H), 4.80-4.50 (m, 2H), 4.35-3.72 (m, 4H), 3.68-3.35 (m, 20H), 3.29-2.97 (m, 1H), 2.70-2.64 (m, 3H), 2.42 (s, 3H), 2.35-2.11 (m, 2H), 2.10-1.88 (m, 1H), 1.68 (s, 3H). LC-MS: MS (ES⁺): RT=2.728 min, m/z=1028.2 [M+H⁺]; LC-MS method 10.

### Example 26—Synthesis of Compound I-26

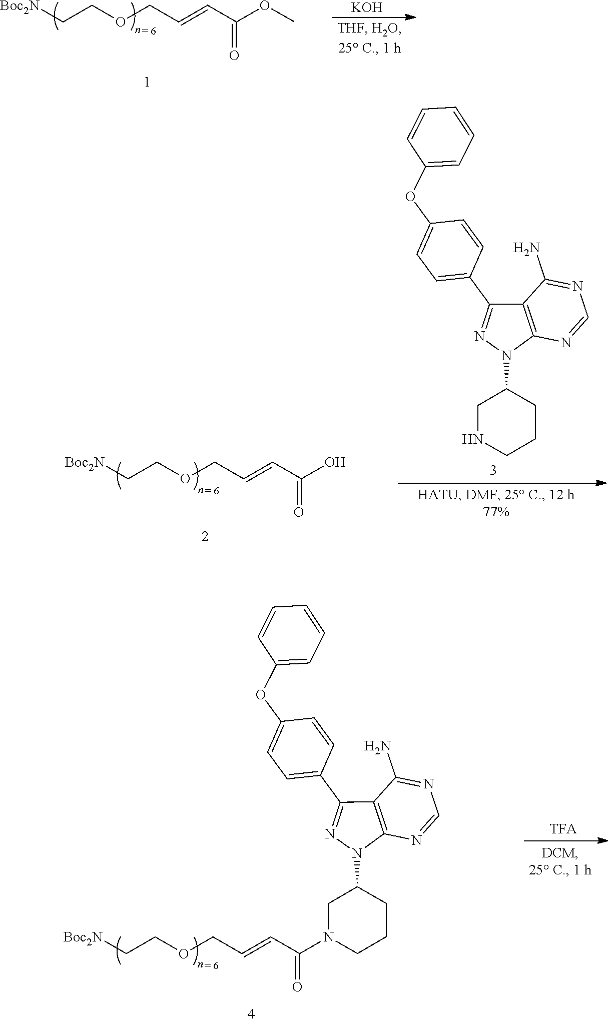

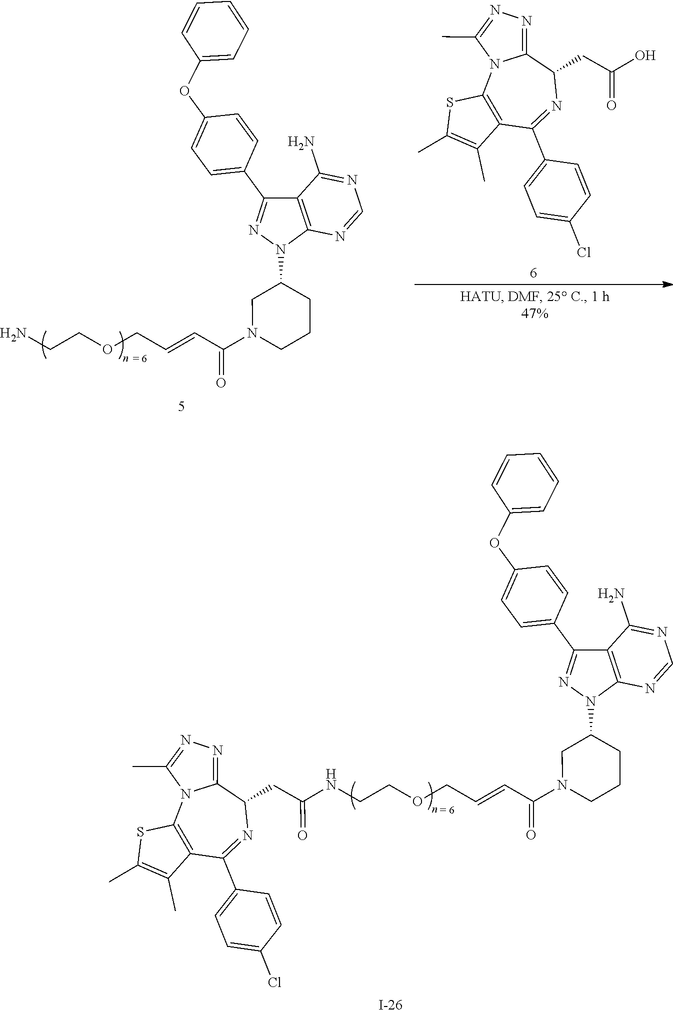

### 26.1 Preparation of Compound 2

To a solution of methyl-(E)-4-[2-[2-[2-[2-[2-[2-[bis(tertbutoxycarbonyl)-amino]ethoxy]ethoxy]-ethoxy]ethoxy]ethoxy]ethoxy]but-2-enoate (930 mg, 1.60 mmol, 1.0 equiv) in THF (10 mL) and H₂O (5.0 mL) was added KOH (135 mg, 2.41 mmol, 1.5 equiv). The mixture was stirred at 25° C. for 1 h. The mixture was concentrated to give crude product (E)-4-[2-[2-[2-[2-[2-[2-[bis(tert-butoxycarbonyl)amino]ethoxy]ethoxy]ethoxy]ethoxy]ethoxy]ethoxy]but-2-enoic acid (920 mg, crude) as a brown oil. LC-MS: MS (ES⁺): RT=1.099 min, m/z=366.2 [M+H⁺-200].

### 26.2 Preparation of Compound 4

To a solution of 3-(4-phenoxyphenyl)-1-[(3R)-3-piperidyl]pyrazolo[3,4-d]pyrimidin-4-amine (510 mg, 1.32 mmol, 1.0 equiv) in DMF (5 mL) were added HATU (602 mg, 1.58 mmol, 1.2 equiv), (E)-4-[2-[2-[2-[2-[2-[2-[bis(tertbutoxycarbonyl)amino]ethoxy]ethoxy]ethoxy]ethoxy]ethoxy]ethoxy]but-2-enoic acid (896 mg, 1.58 mmol, 1.2 equiv) and DIEA (341 mg, 2.64 mmol, 2.0 equiv). The mixture was stirred at 25° C. for 12 h. The solution was purified by prep-HPLC (column: Waters Xbridge C18 150*50 mm*10 μm; mobile phase: [water (10 mM NH₄HCO₃)-ACN]; B %: 53%-83%, 10 min) to give tert-butyl-N-[2-[2-[2-[2-[2-[2-[(E)-4-[(3R)-3-[4-amino-3-(4-phenoxyphenyl) pyrazolo[3,4-d]pyrimidin-1-yl]-1-piperidyl]-4-oxo-but-2-enoxy]ethoxy]ethoxy]ethoxy]ethoxy]ethoxy]ethyl]-N-tert-butoxycarbonyl-carbamate (950 mg, 1.02 mmol, 77% yield) as a white solid. LC-MS: MS (ES⁺): RT=1.100 min, m/z=934.5 [M+H⁺].

### 26.3 Preparation of Compound 5

Prepared similarly to compound 4 in Compound I-24. The mixture was concentrated to give crude (E)-4-[2-[2-[2-[2-[2-(2-aminoethoxy)ethoxy]ethoxy]ethoxy]-ethoxy]ethoxy]-1-[(3R)-3-[4-amino-3-(4-phenoxyphenyl) pyrazolo[3,4-d]pyrimidin-1-yl]-1-piperidyl]but-2-en-1-one (90 mg, crude, TFA salt) a colorless gum. LC-MS: MS (ES⁺): RT=1.158 min, m/z=734.4 [M+H⁺].

### 26.4 Preparation of Compound I-26

To a solution of (E)-4-[2-[2-[2-[2-[2-(2-aminoethoxy)ethoxy]ethoxy]ethoxy]ethoxy]ethoxy]-1-[(3R)-3-[4-amino-3-(4-phenoxyphenyl) pyrazolo[3,4-d]pyrimidin-1-yl]-1-piperidyl]but-2-en-1-one (90.0 mg, 106 mmol, 1.0 equiv, TFA salt) and 2-[(9S)-7-(4-chlorophenyl)-4,5,13-trimethyl-3-thia-1,8,11,12-tetrazatricyclo[8.3.0.0^{2,6}]trideca-2 (6), 4,7,10, 12-pentaen-9-yl]acetic acid (43 mg, 0.11 mmol, 1.0 equiv) in DMF (2 mL) was added DIEA (68 mg, 0.53 mmol, 5.0 equiv) and HATU (48 mg, 0.13 mmol, 1.2 equiv). The mixture was stirred at 25° C. for 1 h. The mixture was purified by prep-HPLC (column: Waters Xbridge 150*25 mm*5 μm; mobile phase: [water (10 mM NH₄HCO₃)-ACN]; B %: 44%-74%, 8 min) to give N-[2-[2-[2-[2-[2-[2-[(E)-4-[(3R)-3-[4-amino-3-(4-phenoxyphenyl) pyrazolo[3,4-d]pyrimidin-1-yl]-1-piperidyl]-4-oxo-but-2-enoxy]ethoxy]ethoxy]ethoxy]ethoxy]ethoxy]ethyl]-2-[(9S)-7-(4-chlorophenyl)-4,5, 13-trimethyl-3-thia-1,8, 11,12-tetrazatricyclo[8.3.0.0^{2,6}]trideca-2 (6),4,7,10, 12-pentaen-9-yl]acetamide (56 mg, 50 mmol, 47% yield, 100% purity) as a off-white solid. ¹H NMR (400 MHZ, MeOD): δ 8.32-8.16 (m, 1H), 7.67 (d, J=8.4 Hz, 2H), 7.48-7.34 (m, 6H), 7.23-7.01 (m, 5H), 6.82-6.44 (m, 2H), 4.81-4.50 (m, 2H), 4.22-3.89 (m, 4H), 3.72-3.34 (m, 27H), 3.30-3.23 (m, 1H), 2.71-2.65 (m, 3H), 2.42 (s, 3H), 2.38-1.99 (m, 3H), 1.68 (s, 4H). LC-MS: MS (ES⁺): RT=2.749 min, m/z=1117.4 [M+H⁺]; LC-MS method 10.

### Example 27—Synthesis of Compound I-27

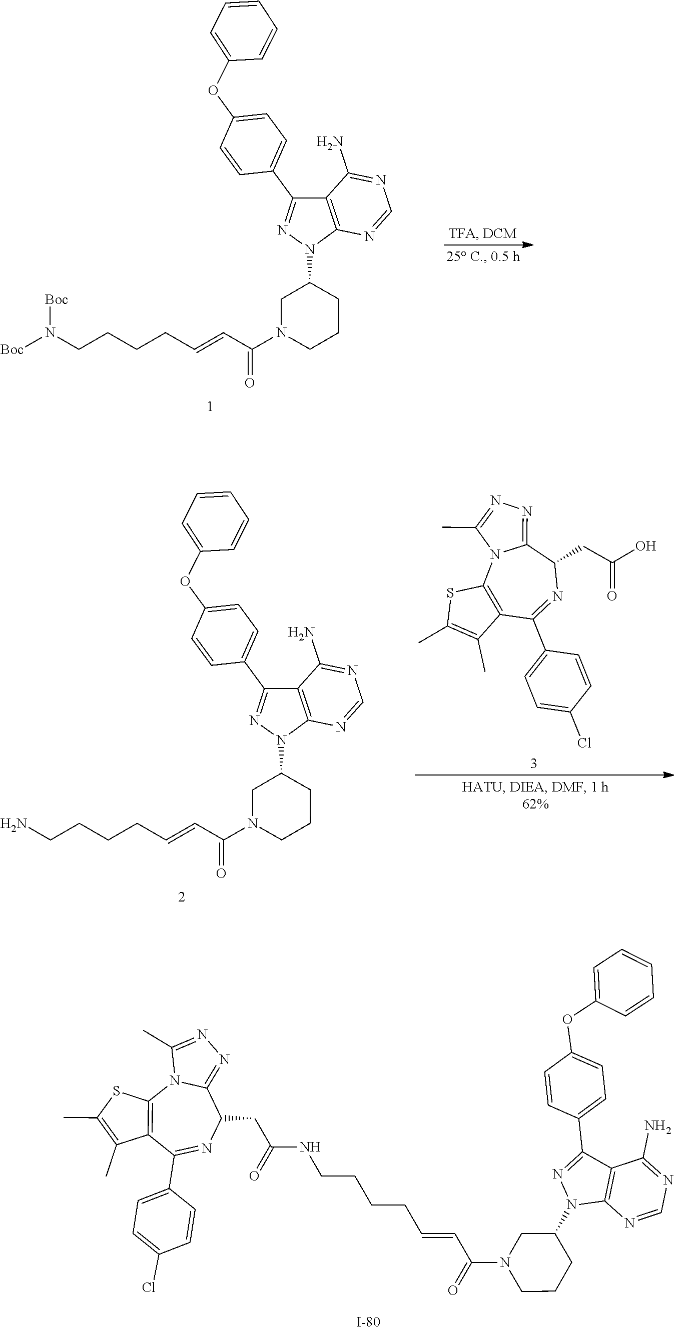

### 27.1 Preparation of Compound 2

To a solution of tert-butyl-N—[(E)-7-[(3R)-3-[4-amino-3-(4-phenoxyphenyl) pyrazolo[3,4-d]pyrimidin-1-yl]-1-piperidyl]-7-oxo-hept-5-enyl]-N-tert-butoxycarbonyl-carbamate (200 mg, 280 mmol, 1.0 equiv) in DCM (1.5 mL) was added TFA (770 mg, 6.75 mmol, 0.5 mL, 24.0 equiv). The mixture was stirred at 25° C. for 0.5 h. The mixture was concentrated to give crude (E)-7-amino-1-[(3R)-3-[4-amino-3-(4-phenoxyphenyl) pyrazolo[3,4-d]pyrimidin-1-yl]-1-piperidyl]hept-2-en-1-one (170 mg, 271 mmol, 96% yield, TFA salt) as a yellow oil. LC-MS: MS (ES⁺): RT=0.707 min, m/z=512.2 [M+H⁺].

### 27.2 Preparation of I-27

To a solution of (E)-7-amino-1-[(3R)-3-[4-amino-3-(4-phenoxyphenyl) pyrazolo[3,4-d]pyrimidin-1-yl]-1-piperidyl]hept-2-en-1-one (78 mg, 0.12 mmol, 1.0 equiv, TFA salt) and 2-[(9S)-7-(4-chlorophenyl)-4,5,13-trimethyl-3-thia-1,8,11,12-tetrazatricyclo[8.3.0.0^{2,6}]trideca-2 (6),4,7,10,12-pentaen-9-yl]acetic acid (50 mg, 0.12 mmol, 1.0 eq) in DMF (1 mL) were added HATU (61 mg, 0.16 mmol, 1.3 equiv) and DIEA (48 mg, 0.37 mmol, 3.0 equiv). The mixture was stirred at 25° C. for 1 h. The solution was purified by prep-HPLC (column: Waters

Xbridge 150*25 mm*5 μm; mobile phase: [water (10 mM NH₄HCO₃)-ACN]; B %: 48%-78%, 10 min) to give N—[(E)-7-[(3R)-3-[4-amino-3-(4-phenoxyphenyl) pyrazolo[3,4-d]pyrimidin-1-yl]-1-piperidyl]-7-oxo-hept-5-enyl]-2-[(9S)-7-(4-chlorophenyl)-4,5,13-trimethyl-3-thia-1,8,11,12-tetrazatricyclo[8.3.0.0^{2,6}]trideca-2 (6),4,7,10, 12-pentaen-9-yl]acetamide (69 mg, 77 mmol, 62% yield, 100% purity) as a white solid. ¹H NMR (400 MHZ, METHANOL-d₄): δ 8.22 (t, J=8.0 Hz, 1H), 7.66 (t, J=8.0 Hz, 1H), 7.49-7.27 (m, 6H), 7.24-6.99 (m, 5H), 6.85-6.62 (m, 1H), 6.57-6.19 (m, 1H), 4.78 (d, J=5.2 Hz, 2H), 4.72-4.18 (m, 2H), 4.07 (d, J=11.2 Hz, 1H), 3.97-3.62 (m, 1H), 3.46-3.34 (m, 2H), 3.28-3.12 (m, 3H), 2.73-2.65 (m, 3H), 2.43 (s, 3H), 2.33-1.99 (m, 4H), 1.74-1.38 (m, 8H). LC-MS: MS (ES⁺): RT=2.819 min, m/z=894.2 [M+H⁺]; LC-MS method 10.

### Example 28—Synthesis of Compound I-28

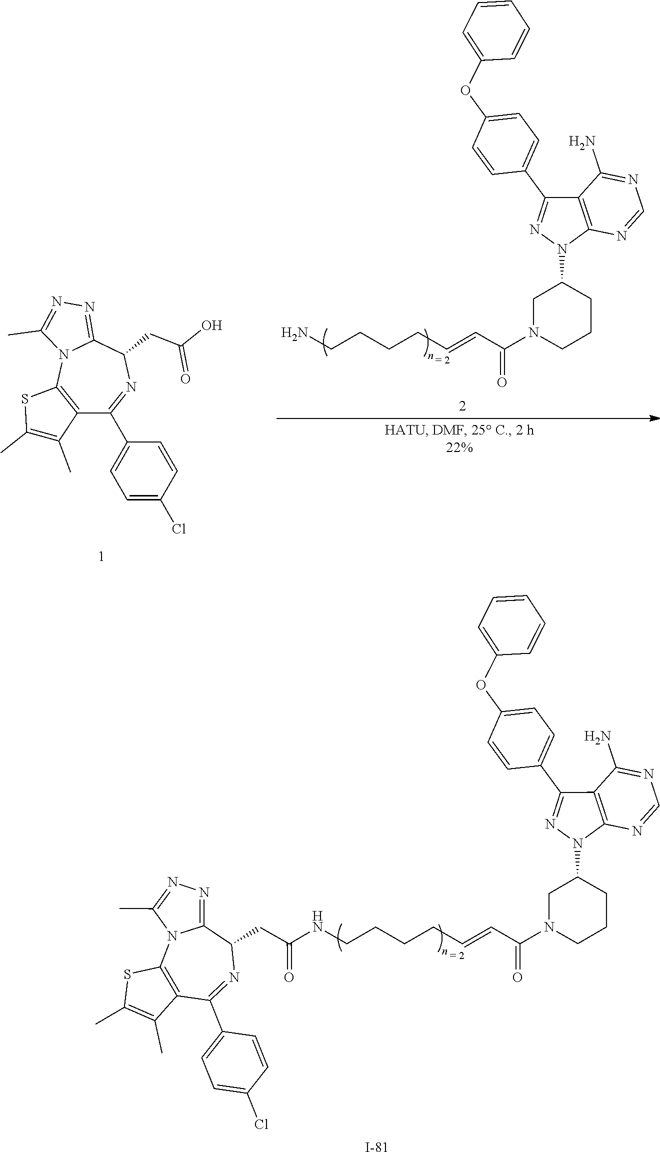

To a solution of (E)-11-amino-1-[(3R)-3-[4-amino-3-(4-phenoxyphenyl) pyrazolo[3,4-d]pyrimidin-1-yl]-1-piperidyl]undec-2-en-1-one (124 mg, 181 mmol, 1.0 equiv, TFA salt) and 2-[(9S)-7-(4-chlorophenyl)-4,5,13-trimethyl-3-thia-1,8,11,12-tetrazatricyclo[8.3.0.0^{2,6}]trideca-2 (6),4, 7,10, 12-pentaen-9-yl]acetic acid (72 mg, 0.18 mmol, 1.0 equiv) in DMF (2 mL) were added DIEA (118 mg, 909 mmol, 5.0 equiv) and HATU (83 mg, 0.22 mmol, 1.2 equiv). The mixture was stirred at 25° C. for 2 h. The mixture was purified by prep-HPLC (column: Phenomenex Gemini-NX C18 75*30 mm*3 μm; mobile phase: [water (10 mM NH₄HCO₃)-ACN]; B %: 52%-82%, 8 min) to give N—[(E)-11-[(3R)-3-[4-amino-3-(4-phenoxyphenyl) pyrazolo[3,4-d]pyrimidin-1-yl]-1-piperidyl]-11-oxo-undec-9-enyl]-2-[(9S)-7-(4-chlorophenyl)-4,5,13-trimethyl-3-thia-1,8,11,12-tetrazatricyclo[8.3.0.0^{2,6}]trideca-2 (6),4,7,10,12-pentaen-9-yl]acetamide (38 mg, 40 mmol, 22% yield, 100% purity) as a white solid. ¹H NMR (400 MHZ, MeOD): δ 8.29-8.19 (m, 1H), 7.73-7.61 (m, 2H), 7.52-7.29 (m, 6H), 7.23-7.03 (m, 5H), 6.84-6.59 (m, 1H), 6.53-6.14 (m, 1H), 4.70-3.97 (m, 4H), 3.59-3.37 (m, 2H), 3.29-3.06 (m, 3H), 2.68 (s, 3H), 2.43 (s, 3H), 2.35-1.97 (m, 5H), 1.74-1.14 (m, 17H). LC-MS: MS (ES⁺): RT=2.803 min, m/z=950.3 [M+H⁺]; LC-MS method 25.

### Example 29—Synthesis of Compound I-29

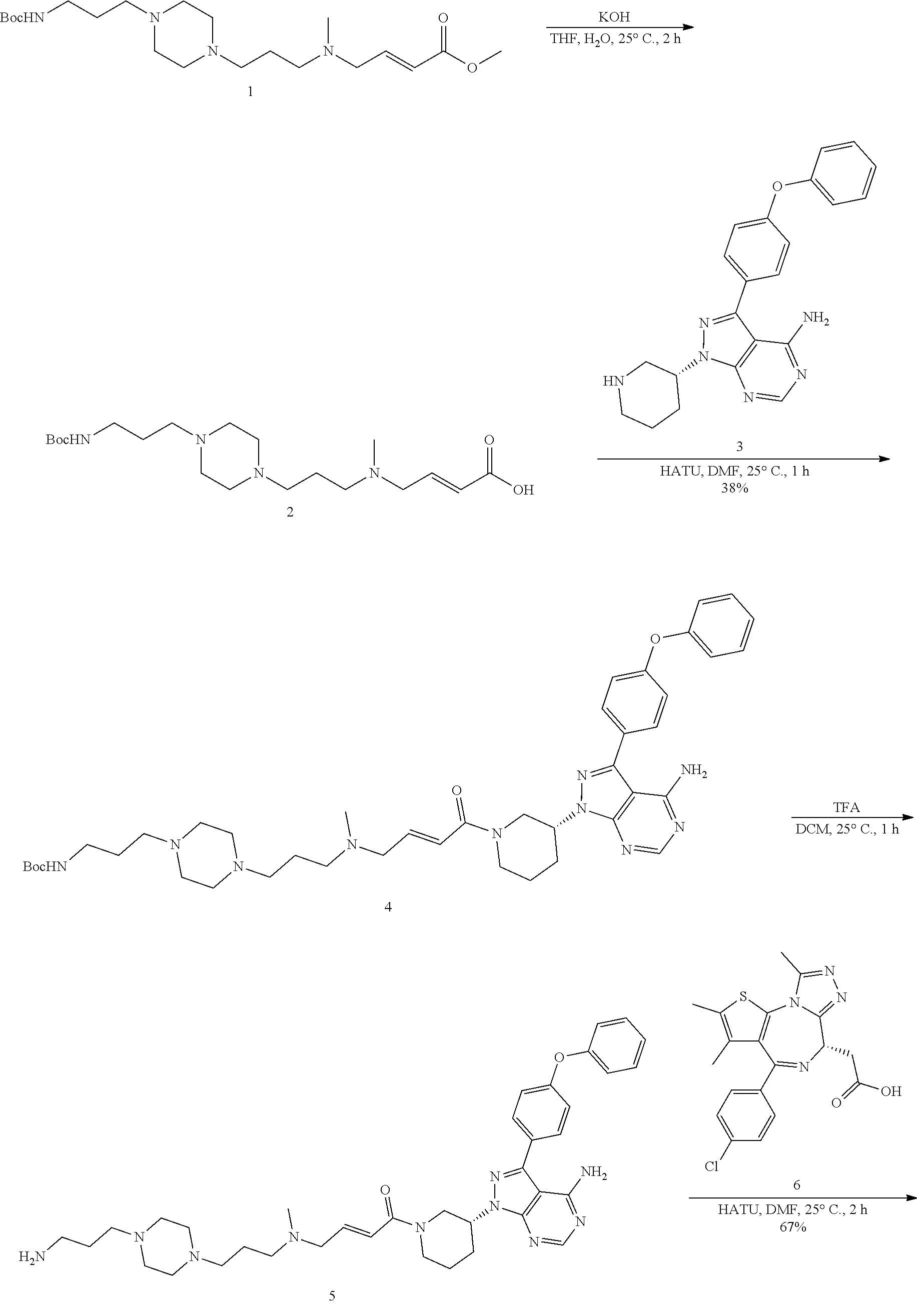

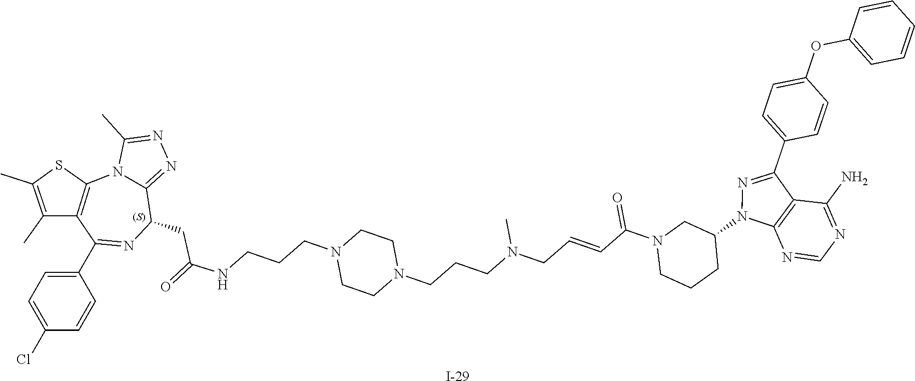

### 29.1 Preparation of Compound 2

To a solution of methyl-(E)-4-[3-[4-[3-(tert-butoxycarbonylamino) propyl]piperazin-1-yl]propyl-methyl-amino]but-2-enoate (200 mg, 485 mmol, 1.0 equiv) in THF (3 mL) and H₂O (1.5 mL) was added KOH (81 mg, 1.5 mmol, 3.0 equiv). The mixture was stirred at 25° C. for 2 h. The mixture was concentrated to give crude (E)-4-[3-[4-[3-(tert-butoxycarbonylamino) propyl]piperazin-1-yl]propyl-methyl-amino]but-2-enoic acid (193 mg, crude) as a white solid. LC-MS: MS (ES⁺): RT=0.727 min, m/z=399.3 [M+H⁺].

### 29.2 Preparation of Compound 4

Prepared similarly to compound 3 in Compound I-24. The mixture was purified by prep-HPLC (column: Phenomenex Gemini-NX C18 75*30 mm*3 μm; mobile phase: [water (10 mM NH₄HCO₃)-ACN]; B %: 35%-65%, 8 min) to give tert-butyl-N-[3-[4-[3-[[(E)-4-[(3R)-3-[4-amino-3-(4-phenoxyphenyl) pyrazolo[3,4-d]-pyrimidin-1-yl]-1-piperidyl]-4-oxo-but-2-enyl]-methyl-amino]propyl]piperazin-1-yl]propyl]carbamate (150 mg, 182 mmol, 38% yield, 93% purity) as a white solid. LC-MS: MS (ES⁺): RT=1.033 min, m/z=767.5 [M+H⁺];

### 29.3 Preparation of Compound 5

Prepared similarly to compound 4 in Compound I-24. The mixture was concentrated to give crude (E)-1-[(3R)-3-[4-amino-3-(4-phenoxyphenyl) pyrazolo[3,4-d]pyrimidin-1-yl]-1-piperidyl]-4-[3-[4-(3-aminopropyl) piperazin-1-yl]propyl-methyl-amino]but-2-en-1-one (101 mg, crude, TFA salt) as a yellow gum. LC-MS: MS (ES⁺): RT =0.525 min, m/z=667.4 [M+H⁺];

### 29.4 Preparation of I-29

To a solution of (E)-1-[(3R)-3-[4-amino-3-(4-phenoxyphenyl) pyrazolo[3,4-d]pyrimidin-1-yl]-1-piperidyl]-4-[3-[4-(3-aminopropyl) piperazin-1-yl]propyl-methyl-amino]but-2-en-1-one (101 mg, 129 mmol, 1.0 equiv, TFA salt) and 2-[(9S)-7-(4-chlorophenyl)-4,5,13-trimethyl-3-thia-1,8,11,12-tetrazatricyclo[8.3.0.0^{2,6}]trideca-2 (6),4,7,10, 12-pentaen-9-yl]acetic acid (52 mg, 0.13 mmol, 1.0 equiv) in DMF (2 mL) were added DIEA (84 mg, 0.65 mmol, 5.0 equiv) and HATU (59 mg, 0.16 mmol, 1.2 equiv). The mixture was stirred at 25° C. for 2 h. The mixture was purified by prep-HPLC (column: Unisil 3-100 C18 Ultra 150*50 mm*3 μm; mobile phase: [water (0.225% FA)-ACN]; B %: 20%-50%, 10 min) to give N-[3-[4-[3-[[(E)-4-[(3R)-3-[4-amino-3-(4-phenoxyphenyl) pyrazolo[3,4-d]pyrimidin-1-yl]-1-piperidyl]-4-oxo-but-2-enyl]-methyl-amino]propyl]piperazin-1-yl]propyl]-2-[(9S)-7-(4-chlorophenyl)-4,5, 13-trimethyl-3-thia-1,8, 11,12-tetrazatricyclo[8.3.0.0^{2,6}]trideca-2 (6),4,7,10,12-pentaen-9-yl]acetamide (90 mg, 86 mmol, 66% yield, 100% purity) as a off-white solid. LC-MS: MS (ES⁺): RT=1.853 min, m/z=525.3 [1/2M+1]; LC-MS method 25. ¹H NMR (400 MHZ, MeOD): δ 8.24 (d, J=10.4 Hz, 1H), 7.67 (d, J=8.4 Hz, 2H), 750-7.33 (m, 6H), 7.24-7.04 (m, 5H), 6.99-6.55 (m, 2H), 4.81-4.55 (m, 2H), 4.27-4.14 (m, 1H), 4.13-3.84 (m, 1H), 3.81-3.64 (m, 1H), 3.61-3.35 (m, 5H), 3.30-3.18 (m, 2H), 3.16-2.54 (m, 20H), 2.43 (s, 3H), 2.40-2.28 (m, 1H), 2.23-2.04 (m, 2H), 1.99-1.78 (m, 4H), 1.77-1.62 (m, 4H).

### Example 30—Synthesis of Compound I-30

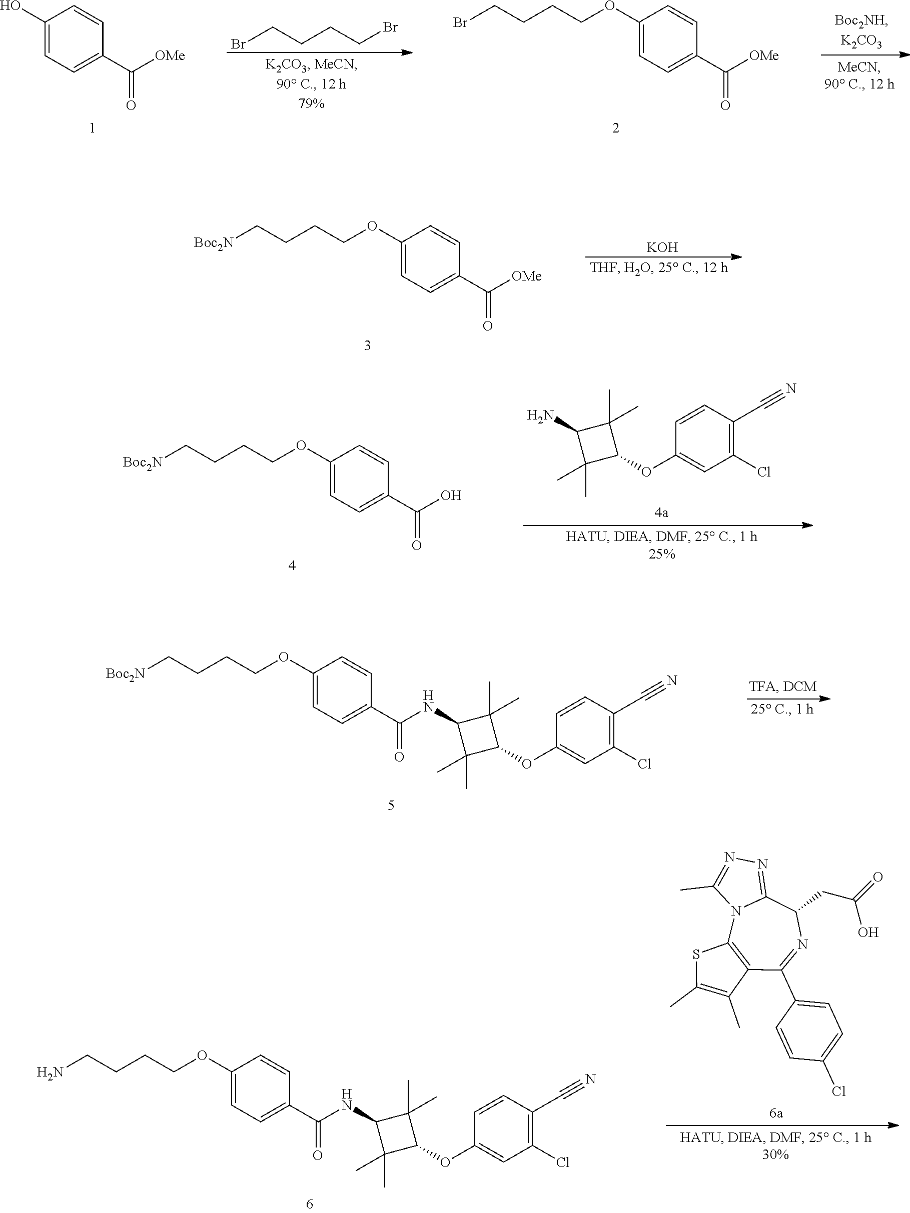

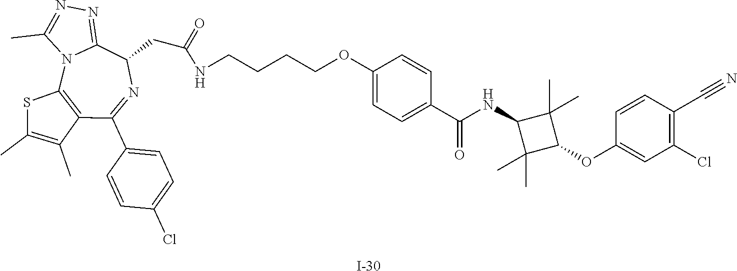

### 30.1 Preparation of Compound 2

To a solution of methyl 4-hydroxybenzoate (3.0 g, 19 mmol, 1.0 equiv) in MeCN (100 mL) were added 1,4-dibromobutane (8.5 g, 39 mmol, 2.0 equiv) and K₂CO₃ (5.5 g, 39 mmol, 2.0 equiv). The mixture was stirred at 90° C. for 12 h. The reaction mixture was filtered. The filtrate was concentrated to give a residue. The residue was purified by column chromatography (SiO₂, petroleum ether: ethyl acetate=100/1 to 10/1) to give methyl 4-(4-bromobutoxy) benzoate (4.5 g, 16 mmol, 79% yield) as a white oil. LC-MS: MS (ES⁺): RT=0.997 min, m/z=289.3 [M+1+H⁺].

### 30.2 Preparation of Compound 3

To a solution of methyl 4-(4-bromobutoxy) benzoate (4.5 g, 16 mmol, 1.0 equiv) in MeCN(100 mL) were added tert-butyl N-tert-butoxycarbonylcarbamate (5.1 g, 24 mmol, 1.5 equiv) and K₂CO₃ (4.3 g, 31 mmol, 2.0 equiv). The mixture was stirred at 90° C. for 12 h. The reaction mixture was filtered. The filtrate was concentrated to give a residue. The residue was purified by column chromatography (SiO₂, petroleum ether: ethyl acetate=100/1 to 10/1) to give methyl-4-[4-[bis(tert-butoxycarbonyl)amino]butoxy]benzoate (8.0 g, crude) as a white oil. ¹H NMR (400 MHZ, CHLOROFORM-d): δ 7.98 (d, J=8.8 Hz, 2H), 6.89 (d, J=8.8 Hz, 2H), 4.03 (t, J=5.9 Hz, 2H), 3.89 (s, 3H), 3.65 (t, J=6.9 Hz, 2H), 1.87-1.72 (m, 4H), 1.51 (s, 18H).

### 30.3 Preparation of 4

To a solution of methyl-4-[4-[bis(tert-butoxycarbonyl)amino]butoxy]benzoate (300 mg, 708 mmol, 1.0 equiv) in THF (3 mL) and H₂O (1 mL) was added KOH (60 mg, 1.1 mmol, 1.5 equiv). The mixture was stirred at 25° C. for 12 h. The pH of mixture was adjusted to 5 with HCl (1 M, aq.). The mixture was extracted with ethyl acetate (3×10 mL). The combined organic layers were washed with brine (10 mL), dried over by sodium sulfate and filtered. The filtrate was concentrated to give 4-[4-[bis(tert-butoxycarbonyl)amino]butoxy]benzoic acid (180 mg, 440 mmol) as a white solid.

### 30.4 Preparation of Compound 5

Prepared similarly to compound 3 in Compound I-24. The solution was purified by prep-HPLC (column: Waters Xbridge 150*25 mm*5 μm; mobile phase: [water (10 mM NH₄HCO₃)-ACN]; B %: 68%-98%, 8 min) to give tert-butyl-N-tert-butoxycarbonyl-N-[4-[4-[[3-(3-chloro-4-cyano-phenoxy)-2,2,4,4-tetramethyl-cyclobutyl]carbamoyl]phenoxy]butyl]carbamate (75 mg, 0.11 mmol, 25% yield) as a white solid. LC-MS: MS (ES⁺): RT=1.113 min, m/z=570.5 [M+H⁺−100].

### 30.5 Preparation of Compound 6

Prepared similarly to compound 4 in Compound I-24. The mixture was concentrated to give the crude 4-(4-aminobutoxy)-N-[3-(3-chloro-4-cyano-phenoxy)-2,2,4,4-tetramethyl-cyclobutyl]benzamide (60 mg, 0.10 mmol, TFA salt) as a yellow oil. LC-MS: MS (ES⁺): RT=0.855 min, m/z=470.5 [M+H⁺].

### 30.6 Preparation of Compound I-30

To a solution of 4-(4-aminobutoxy)-N-[3-(3-chloro-4-cyano-phenoxy)-2,2,4,4-tetramethyl-cyclobutyl]benzamide (60 mg, 0.10 mmol, 1.0 equiv, TFA salt) in DMF (1 mL) were added HATU (51 mg, 0.13 mmol, 1.3 equiv), 2-[7-(4-chlorophenyl)-4,5,13-trimethyl-3-thia-1,8, 11,12-tetrazatricyclo[8.3.0.0^{2,6}]trideca-2 (6),4,7, 10, 12-pentaen-9-yl]acetic acid (41 mg, 0.10 mmol, 1.0 equiv) and DIEA (53 mg, 0.41 mmol, 4.0 equiv). The mixture was stirred at 25° C. for 1 h. The solution was purified by prep-HPLC (column: Phenomenex Synergi C18 150*25 mm*10 μm; mobile phase: [water (0.225% FA)-ACN]; B %: 68%-95%, 9 min) to give N-[3-(3-chloro-4-cyano-phenoxy)-2,2,4,4-tetramethyl-cyclobutyl]-4-[4-[2-[(9S)-7-(4-chlorophenyl)-4,5,13-trimethyl-3-thia-1,8,11,12-tetrazatricyclo[8.3.0.0^{2,6}]trideca-2 (6),4,7,10,12-pentaen-9-yl]acetyl]amino]butoxy]benzamide (26 mg, 30 mmol, 30% yield, 100% purity) as a white solid. ¹H NMR (400 MHZ, METHANOL-d₄): δ 7.80 (d, J=8.2 Hz, 2H), 7.72 (d, J=8.6 Hz, 1H), 7.45 (d, J=8.0 Hz, 2H), 7.35 (d, J=8.2 Hz, 2H), 7.13 (s, 1H), 6.99 (d, J=7.8 Hz, 3H), 4.64 (dd, J=5.4, 8.6 Hz, 1H), 4.29 (s, 1H), 4.17-4.12 (m, 1H), 4.09 (t, J=5.4 Hz, 2H), 3.48-3.36 (m, 2H), 3.33 (s, 1H), 3.27 (d, J=5.6 Hz, 1H), 2.69 (s, 3H), 2.44 (s, 3H), 1.95-1.83 (m, 2H), 1.83-1.72 (m, 2H), 1.69 (s, 3H), 1.28 (s, 6H), 1.22 (s, 6H). LC-MS: MS (ES⁺): RT=2.623 min, m/z=852.3 [M+H⁺]; LC-MS method 40.

### Example 31—Synthesis of Compound I-31

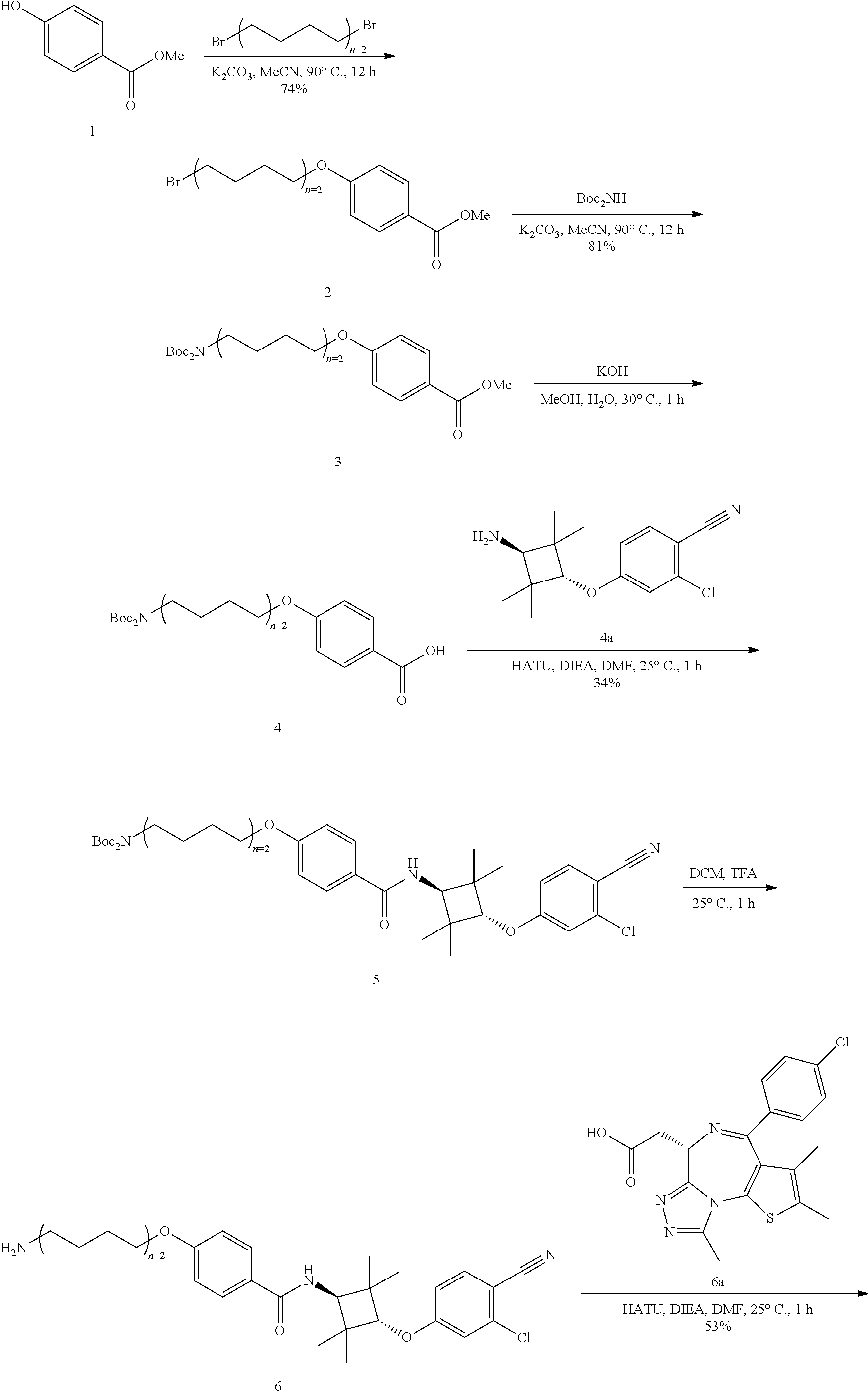

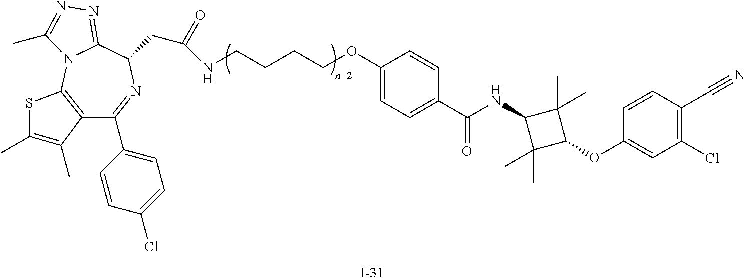

### 31.1 Preparation of Compound 2

Prepared similarly to compound 2 in Compound I-30. The residue was purified by silica column chromatography on silica gel (Petroleum ether: Ethyl acetate=50/1 to 15/1) to give the desired product methyl 4-(8-bromooctoxy) benzoate (5.00 g, 14.6 mmol, 74% yield) as a yellow oil. ¹H NMR (400 MHz, CDCl₃): δ 8.01-7.95 (m, 2H), 6.94-6.88 (m, 2H), 4.01 (t, J=6.4 Hz, 2H), 3.89 (s, 3H), 3.42 (t, J=6.8 Hz, 2H), 1.89-1.76 (m, 3H), 1.77-1.76 (m, 1H), 1.49-1.35 (m, 8H). LC-MS: MS (ES⁺): RT=1.137 min, m/z=345.0 [M+H⁺].

### 31.2 Preparation of Compound 3

Prepared similarly to compound 3 in Compound I-30. The residue was purified by silica column chromatography on silica gel (Petroleum ether: Ethyl acetate from 80/1 to 30/1) to give the desired product methyl 4-[8-[bis(tert-butoxycarbonyl)amino]octoxy]benzoate (5.64 g, 11.8 mmol, 81% yield) as a yellow oil. ¹H NMR (400 MHZ, CDCl₃): δ 7.97 (d, J=8.9 Hz, 2H), 6.89 (d, J=8.8 Hz, 2H), 3.99 (t, J=6.5 Hz, 2H), 3.87 (s, 3H), 3.58-3.51 (m, 2H), 1.82-1.75 (m, 2H), 1.60-1.53 (m, 2H), 1.49 (s, 18H), 1.45 (d, J=2.1 Hz, 2H), 1.34 (d, J=3.3 Hz, 6H).

### 31.3 Preparation of Compound 4

To a solution of methyl 4-[8-[bis(tert-butoxycarbonyl)amino]octoxy]benzoate (120 mg, 250 mmol, 1.0 equiv) in MeOH (2 mL) and H₂O (1 mL) was added KOH (21.1 mg, 375 mmol, 1.5 equiv) and. The mixture was stirred at 30° C. for 1.0 h. The mixture was concentrated to give the crude product 4-[8-[bis(tert-butoxycarbonyl)amino]octoxy]benzoic acid (115 mg, crude) as a white solid.

### 31.4 Preparation of Compound 5

Prepared similarly to compound 3 in Compound I-24. The residue was purified by prep-HPLC (column: 3_Phenomenex Luna C18 75*30 mm*3 μm; mobile phase: [water (0.1% TFA)-ACN]; B %: 75%-100%, 8 min) to give the desired product tert-butyl N-tert-butoxycarbonyl-N-[8-[4-[3-(3-chloro-4-cyano-phenoxy)-2,2,4,4-tetramethyl-cyclobutyl]carbamoyl]phenoxy]octyl]carbamate (50 mg, 69 mmol, 34% yield) as a white solid. LC-MS: MS (ES⁺): RT=0.908 min, m/z=626.2 [M−100⁺].

### 31.5 Preparation of Compound 6

Prepared similarly to compound 4 in Compound I-24. The mixture was concentrated to give the crude product 4-(8-aminooctoxy)-N-[3-(3-chloro-4-cyano-phenoxy)-2,2,4,4-tetramethyl-cyclobutyl]benzamide (45 mg, crude, TFA salt) as a colorless oil. LC-MS: MS (ES⁺): RT=0.675 min, m/z=526.3 [M+H⁺].

### 31.6 Preparation of Compound I-31

To a solution of 2-[(9S)-7-(4-chlorophenyl)-4,5, 13-trimethyl-3-thia-1,8, 11,12-tetrazatricyclo[8.3.0.0^{2,6}]trideca-2 (6),4,7, 10,12-pentaen-9-yl]acetic acid (28 mg, 70 mmol, 1.0 equiv) in DMF (1 mL) was added HATU (32 mg, 84 mmol, 1.2 equiv), 4-(8-aminooctoxy)-N-[3-(3-chloro-4-cyano-phenoxy)-2,2,4,4-tetramethyl-cyclobutyl]benzamide (45 mg, 70 mmol, 1 equiv, TFA salt) and DIEA (45 mg, 0.4 mmol, 5.0 equiv). The mixture was stirred at 25° C. for 1.0 h. The mixture was concentrated to give a residue. The residue was purified by prep-HPLC (column: Unisil 3-100 C18 Ultra 150*50 mm*3 μm; mobile phase: [water (0.225% FA)-ACN]; B %: 75%-100%, 10 min) to give the desired product N-[3-(3-chloro-4-cyano-phenoxy)-2,2,4,4-tetramethyl-cyclobutyl]-4-[8-[2-[(9S)-7-(4-chlorophenyl)-4,5,13-trimethyl-3-thia-1,8, 11, 12-tetrazatricyclo[8.3.0.0^{2,6}]trideca-2 (6),4,7,10, 12-pentaen-9-yl]acetyl]amino]octoxy]benzamide (34 mg, 37 mmol, 53% yield, 100% purity) as an off-white solid. ¹H NMR (400 MHZ, DMSO-d₆): δ 8.19 (t, J=5.5 Hz, 1H), 7.91 (d, J=8.8 Hz, 1H), 7.82 (d, J=8.8 Hz, 2H), 7.68 (d, J=9.1 Hz, 1H), 7.52-7.38 (m, 4H), 7.21 (d, J=2.4 Hz, 1H), 7.03-6.96 (m, 3H), 4.50 (dd, J=5.9, 8.2 Hz, 1H), 4.31 (s, 1H), 4.12-3.98 (m, 3H), 3.28-3.06 (m, 4H), 2.59 (s, 3H), 2.40 (s, 3H), 1.75-1.67 (m, 2H), 1.61 (s, 3H), 1.49-1.38 (m, 4H), 1.30 (s, 6H), 1.22 (s, 6H), 1.12 (s, 6H). LC-MS: MS (ES⁺): RT=2.098 min, m/z=909.1 [M+H⁺]; LC-MS method 60.

### Example 32—Synthesis of Compound I-32

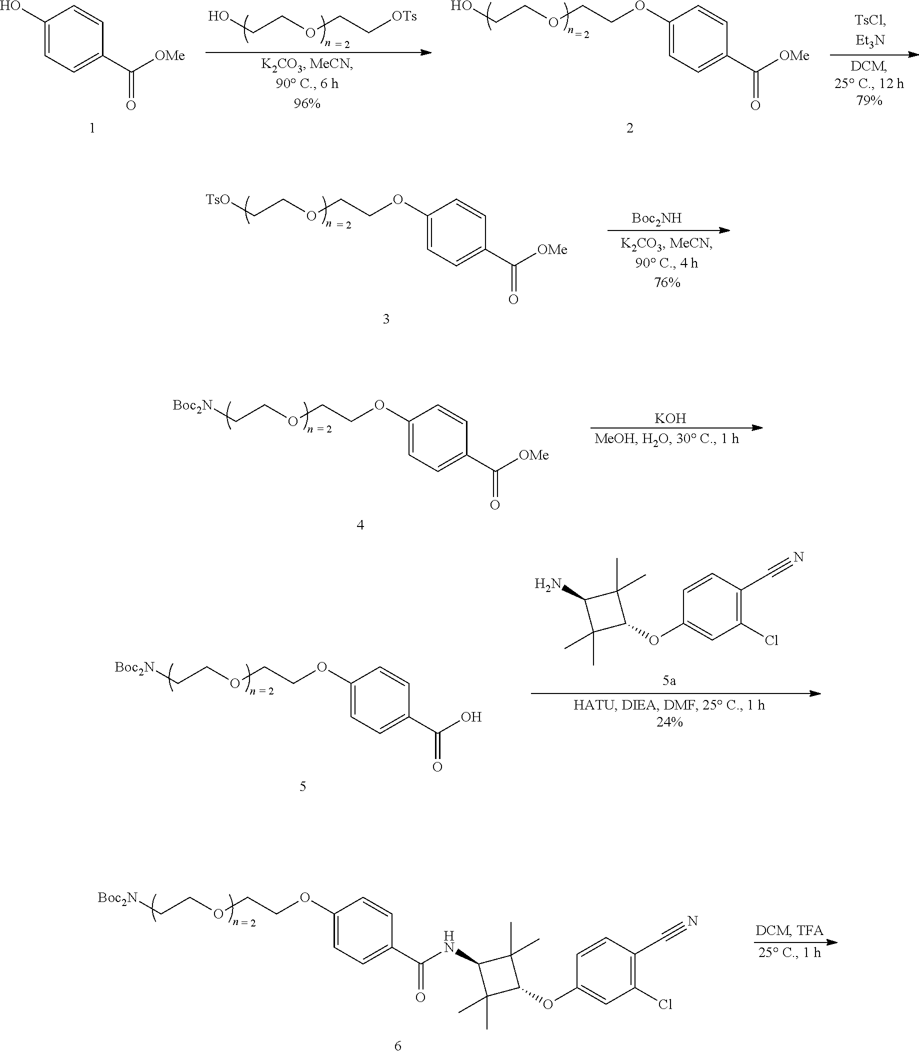

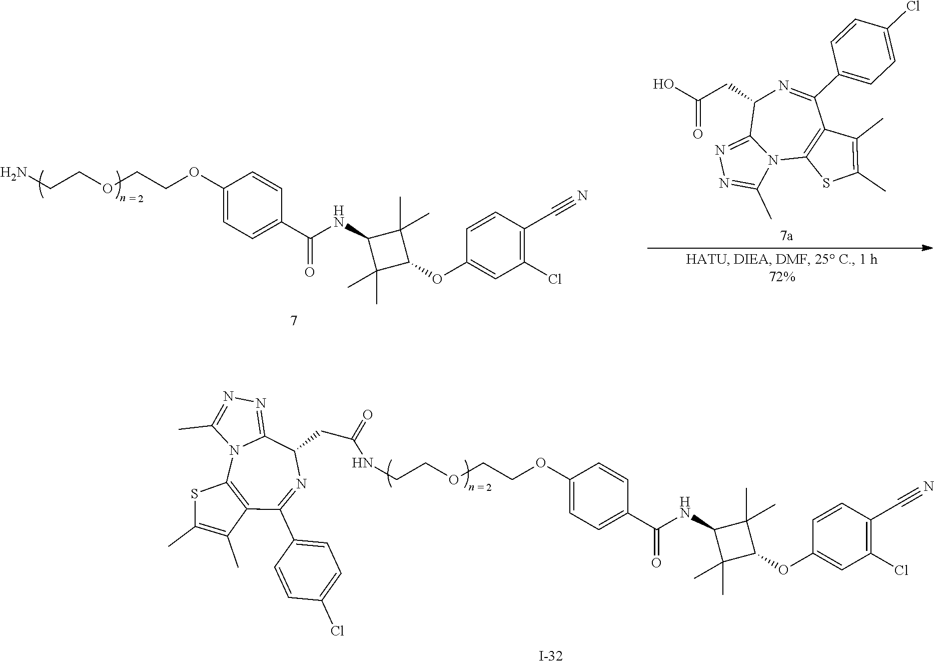

### 32.1 Preparation of Compound 2

To a solution of methyl 4-hydroxybenzoate (1.00 g, 6.57 mmol, 1.0 equiv) and 2-[12-(2-hydroxyethoxy)ethoxy]ethyl 4-methylbenzenesulfonate (2.00 g, 6.57 mmol, 1.0 equiv) in MeCN(20 mL) was added K₂CO₃ (1.82 g, 13.1 mmol, 2.0 equiv). The mixture was stirred at 90° C. for 6 h. The mixture was filtered. The filtrate was concentrated to give a residue. The residue was purified by silica column chromatography on silica gel (Petroleum ether: Ethyl acetate from 20/1 to 0/1) to give the desired product methyl 4-[2-[2-(2-hydroxyethoxy) ethoxy]ethoxy]benzoate (1.80 g, 6.33 mmol, 96% yield) as a yellow oil. ¹H NMR (400 MHZ, CDCl₃): δ 8.01-7.93 (m, 2H), 6.96-6.89 (m, 2H), 4.24-4.14 (m, 3H), 3.76-3.57 (m, 12H). LC-MS: MS (ES⁺): RT=0.808 min, m/z=306.9 [M+Na⁺].

### 32.2 Preparation of Compound 3

To a solution of methyl 4-[2-[2-(2-hydroxyethoxy)ethoxy]ethoxy]benzoate (1.80 g, 6.33 mmol, 1.0 equiv) and 4-methylbenzenesulfonyl chloride (1.57 g, 8.23 mmol, 1.3 equiv) in DCM (15 mL) was added Et₃N(1.28 g, 12.7 mmol, 1.8 mL, 2.0 equiv). The mixture was stirred at 25° C. for 12 h. The mixture was filtered. The filtrate was concentrated to give a residue. The residue was purified by silica column chromatography on silica gel (Petroleum ether: Ethyl acetate from 20/1 to 1/1) to give the desired product methyl 4-[2-[2-[2-(p-tolylsulfonyloxy)ethoxy]ethoxy]ethoxy]benzoate (2.20 g, 5.02 mmol, 79% yield) as a colorless oil. ¹H NMR (400 MHZ, CDCl₃): δ 7.92-7.87 (m, 2H), 7.71 (d, J=8.4 Hz, 2H), 7.25 (d, J=8.0 Hz, 2H), 6.86-6.81 (m, 2H), 4.08 (d, J=4.0 Hz, 2H), 3.80 (s, 3H), 3.78-3.75 (m, 2H), 3.62-3.51 (m, 8H), 2.35 (s, 3H). LC-MS: MS (ES⁺): RT=0.908 min, m/z=439.1 [M+H⁺].

### 32.3 Preparation of Compound 4

To a solution of methyl 4-[2-[2-[2-(p-tolylsulfonyloxy)ethoxy]ethoxy]ethoxy]benzoate (2.20 g, 5.02 mmol, 1.0 equiv) and tert-butyl Ntert-butoxycarbonylcarbamate (1.42 g, 6.52 mmol, 1.3 equiv) in MeCN(50 mL) was added K₂CO₃ (1.39 g, 10.0 mmol, 2.0 equiv). The mixture was stirred at 90° C. for 4 h. The mixture was filtered. The filtrate was concentrated to give a residue. The residue was purified by silica column chromatography on silica gel (Petroleum ether: Ethyl acetate from 20/1 to 10/1) to give the desired product methyl 4-[2-[2-[2-[bis(tert-butoxycarbonyl)amino]ethoxy]ethoxy]ethoxy]benzoate (1.84 g, 3.81 mmol, 76% yield) as a yellow oil. ¹H NMR (400 MHZ, CDCl₃): δ 8.00-7.93 (m, 2H), 6.94-6.89 (m, 2H), 4.18-4.15 (m, 2H), 3.84 (d, J=4.6 Hz, 2H), 3.78 (d, J=6.0 Hz, 2H), 3.69-3.67 (m, 2H), 3.65-3.52 (m, 7H), 1.47 (s, 18H). LC-MS: MS (ES⁺): RT=1.117 min, m/z=506.1 [M+Na⁺].

### 32.4 Preparation of Compound 5

To a solution of methyl 4-[2-[2-[2-[bis(tert-butoxycarbonyl)amino]ethoxy]ethoxy]ethoxy]benzoate (120 mg, 248 mmol, 1.0 equiv) in MeOH (2 mL) and H₂O (1 mL) was added KOH (21.0 mg, 372 mmol, 1.5 equiv). The mixture was stirred at 30° C. for 1.0 h. The mixture was concentrated to give the crude product 4-[2-[2-[2-[bis(tert-butoxycarbonyl)amino]ethoxy]ethoxy]ethoxy]benzoic acid (115 mg, crude) as a white solid.

### 32.5 Preparation of Compound 6

Prepared similarly to compound 3 in Compound I-24. The residue was purified by prep-HPLC (column: 3_Phenomenex Luna C18 75*30 mm*3 μm; mobile phase: [water (0.1% TFA)-ACN]; B %: 62%-92%, 8 min) to give the desired product tert-butyl-N-tert-butoxycarbonyl-N-[2-[2-[2-[4-[[3-(3-chloro-4-cyano-phenoxy)-2,2,4,4-tetramethylcyclobutyl]carbamoyl]phenoxy]ethoxy]ethoxy]ethyl]carbamate (35 mg, 48 mmol, 24% yield) as an off-white solid. LC-MS: MS (ES⁺): RT=0.808 min, m/z=630.2 [M+1-100+].

### 32.6 Preparation of Compound 7

Prepared similarly to compound 4 in Compound I-24. The mixture was concentrated to give the crude product 4-[2-[2-(2-aminoethoxy)ethoxy]ethoxy]-N-[3-(3-chloro-4-cyano-phenoxy)-2,2,4,4-tetramethyl-cyclobutyl]benzamide (32 mg, crude, TFA salt) as a colorless oil. LC-MS: MS (ES⁺): RT=0.617 min, m/z=530.2 [M+H⁺].

### 32.7 Preparation of Compound I-32

To a solution of 2-[(9S)-7-(4-chlorophenyl)-4,5, 13-trimethyl-3-thia-1,8, 11, 12-tetrazatricyclo[8.3.0.0^{2,6}]trideca-2 (6),4,7,10, 12-pentaen-9-yl]acetic acid (20 mg, 50 mmol, 1.0 equiv) in DMF (1 mL) were added 4-[2-[2-(2-aminoethoxy)ethoxy]ethoxy]-N-[3-(3-chloro-4-cyano-phenoxy)-2,2,4,4-tetramethyl-cyclobutyl]benzamide (32 mg, 50 mmol, 1.0 equiv, TFA salt), HATU (23 mg, 60 mmol, 1.2 equiv) and DIEA (32 mg, 0.2 mmol, 5.0 equiv). The mixture was stirred at 25° C. for 1 h. The mixture was concentrated to give a residue. The residue was purified by prep-HPLC (column: Unisil 3-100 C18 Ultra 150*50 mm*3 μm; mobile phase: [water (0.225% FA)-ACN]; B %: 65%-95%, 10 min) to give the desired product N-[3-(3-chloro-4-cyano-phenoxy)-2,2,4,4-tetramethyl-cyclobutyl]-4-[2-[2-[2-[2-[(9S)-7-(4-chlorophenyl)-4,5,13-trimethyl-3-thia-1,8,11, 12-tetrazatricyclo[8.3.0.0^{2,6}]trideca-2 (6) ,4,7,10,12-pentaen-9-yl]acetyl]amino]ethoxy]ethoxy]ethoxy]benzamide (33 mg, 36 mmol, 72% yield, 100% purity) as an off-white solid. ¹H NMR (400 MHZ, DMSO-d₆): δ 8.32 (t, J=5.6 Hz, 1H), 7.91 (d, J=8.8 Hz, 1H), 7.82 (d, J=8.8 Hz, 2H), 7.69 (d, J=9.2 Hz, 1H), 7.53-7.37 (m, 4H), 7.21 (d, J=2.4 Hz, 1H), 7.05-6.96 (m, 3H), 4.55-4.46 (m, 1H), 4.31 (s, 1H), 4.19-4.12 (m, 2H), 4.06 (d, J=9.2 Hz, 1H), 3.80-3.74 (m, 2H), 3.64-3.60 (m, 2H), 3.59-3.55 (m, 2H), 3.50-3.45 (m, 2H), 3.30-3.17 (m, 4H), 2.59 (s, 3H), 2.40 (s, 3H), 1.61 (s, 3H), 1.22 (s, 6H), 1.12 (s, 6H). LC-MS: MS (ES⁺): RT=2.573 min, m/z=912.3 [M+H⁺]; LC-MS method 40.

### Example 33—Synthesis of Compound I-33

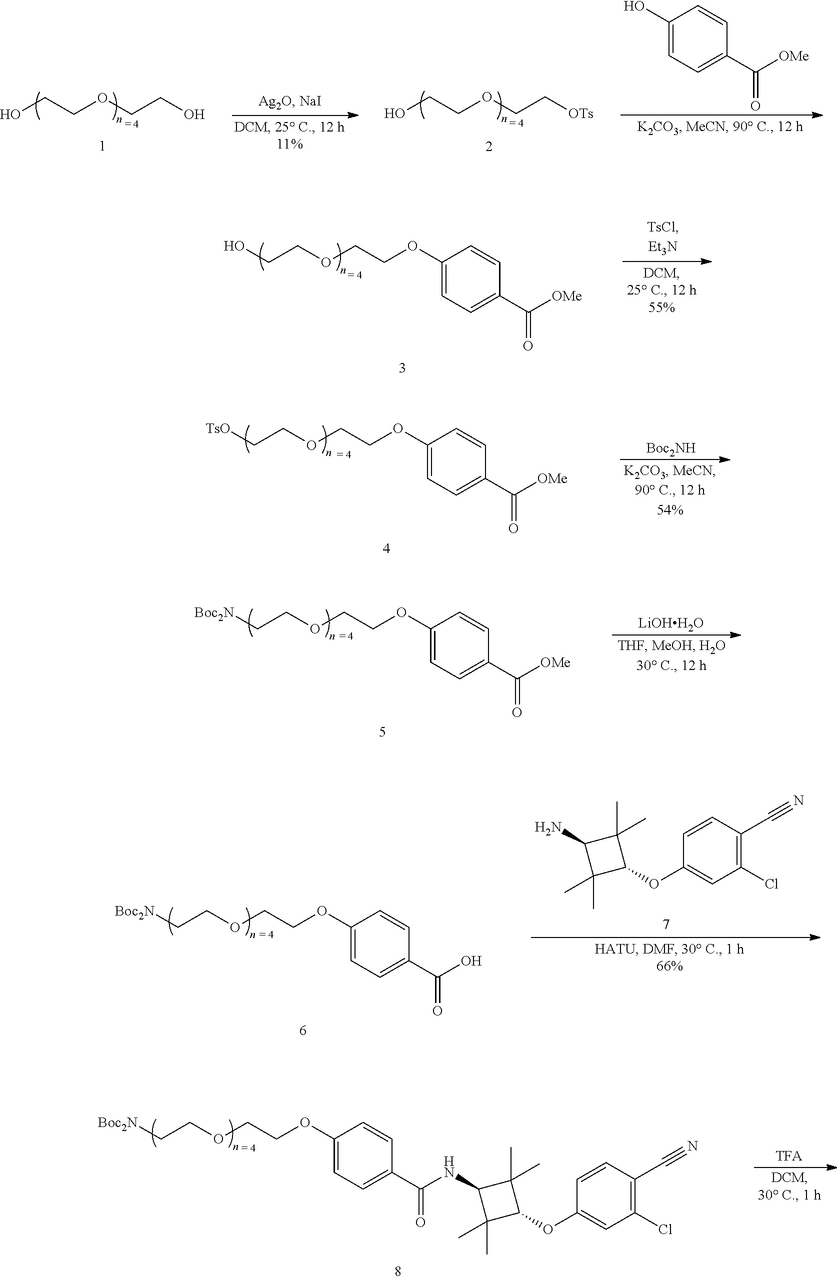

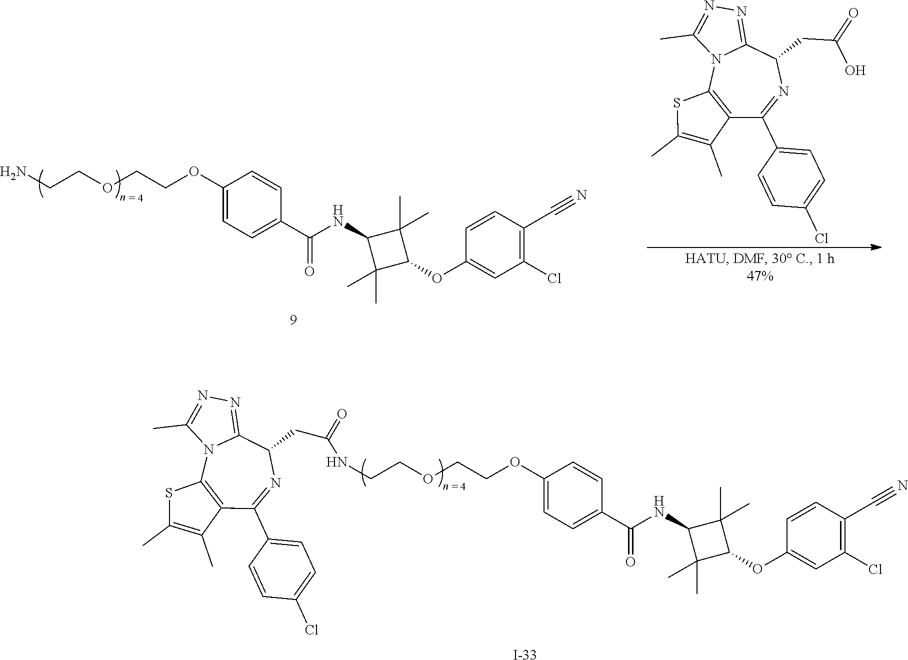

### 33.1 Preparation of Compound 2

To a mixture of 2-[2-[2-[2-(2-hydroxyethoxy)ethoxy]ethoxy]ethoxy]ethanol (10.0 g, 42.0 mmol, 1.0 equiv) in DCM (150 mL) were added Ag₂O (14.6 g, 63.0 mmol, 1.5 equiv), NaI (6.92 g, 46.2 mmol, 1.1 equiv) and 4-methylbenzenesulfonyl chloride (9.60 g, 50.4 mmol, 1.2 equiv) at 0° C. The mixture was stirred at 25° C. for 12 h. The mixture was filtered. The filtrate was concentrated to give a residue. The residue was purified by silica column chromatography (Dichloromethane: Methanol=1:0 to 50:1) to give 2-[2-[2-[2-(2-hydroxyethoxy)ethoxy]ethoxy]ethoxy]ethyl 4-methylbenzenesulfonate (3.70 g, 9.43 mmol, 11% yield) as a yellow solid. LC-MS: MS (ES⁺): RT=0.805 min, m/z=393.4 [M+H⁺].

### 33.2 Preparation of Compound 3

To a solution of methyl 4-hydroxybenzoate (1.43 g, 9.43 mmol, 1.0 equiv) and 2-[2-[2-[2-(2-hydroxyethoxy)ethoxy]ethoxy]ethoxy]ethyl 4-methylbenzenesulfonate (3.70, 9.43 mmol, 1.0 equiv) in MeCN(80 mL) was added K₂CO₃ (2.61 g, 18.9 mmol, 2.0 equiv). The mixture was stirred at 90° C. for 12 h. The mixture was filtered. The filtrate was concentrated to give a residue. The residue was purified by silica column chromatography (Petroleum ether: Ethyl acetate=20/1 to 1/3 then DCM: Methanol=50/1 to 10/1) to give the desired product methyl-4-[2-[2-[2-[2-(2-hydroxyethoxy)ethoxy]ethoxy]ethoxy]ethoxy]benzoate (3.60 g, crude) as a yellow oil. LC-MS: MS (ES⁺): RT=0.800 min, m/z=390.2 [M+18].

### 33.3 Preparation of Compound 4

Prepared similarly to compound 3 in Compound I-32. The residue was purified by silica gel chromatography (Petroleum ether: Ethyl acetate=20/1 to 1/2) to give methyl-4-[2-[2-[2-[2-[2-(p-tolylsulfonyloxy)ethoxy]ethoxy]ethoxy]ethoxy]ethoxy]benzoate (2.80 g, 5.32 mmol, 55% yield) as a yellow oil. LC-MS: MS (ES⁺): RT=0.983 min, m/z=544.2 [M+18].

### 33.4 Preparation of Compound 5

Prepared similarly to compound 4 in Compound I-32. The residue was purified by silica gel chromatography (Petroleum ether: Ethyl acetate=20/1 to 1/2) to give methyl-4-[2-[2-[2-[2-[2-[bis(tert-butoxycarbonyl)amino]ethoxy]ethoxy]ethoxy]ethoxy]ethoxy]benzoate (1.64 g, 2.87 mmol, 54% yield) as a yellow oil. LC-MS: MS (ES⁺): RT=1.108 min, m/z=589.3 [M+18]; 1H NMR (400 MHZ, CDCl₃): δ 7.97 (d, J=8.8 Hz, 2H), 6.92 (d, J=8.8 Hz, 2H), 4.20-4.15 (m, 2H), 3.90-3.85 (m, 5H), 3.79-3.76 (m, 2H), 3.74-3.70 (m, 2H), 3.70-3.56 (m, 15H), 1.50-1.48 (m, 18H).

### 33.5 Preparation of Compound 6

To a solution of methyl-4-[2-[2-[2-[2-[2-[bis(tert-butoxycarbonyl)amino]ethoxy]ethoxy]ethoxy]ethoxy]ethoxy]benzoate (400 mg, 700 mmol, 1.0 equiv) in THF (2 mL), MeOH (2 mL) and H₂O (2 mL) was added LiOH·H₂O (88 mg, 2.1 mmol, 3.0 equiv). The mixture was stirred at 30° C. for 16 h. The mixture was concentrated to give 4-[2-[2-[2-[2-[2-[bis(tert-butoxycarbonyl)amino]ethoxy]ethoxy]ethoxy]ethoxy]ethoxy]benzoic acid (390 mg, crude) as a white solid. LC-MS: MS (ES⁺): RT=0.775 min, m/z=358.1 [M+1-200].

### 33.6 Preparation of Compound 8

To a mixture of 4-[2-[2-[2-[2-[2-[bis(tert-butoxycarbonyl)amino]ethoxy]ethoxy]ethoxy]ethoxy]ethoxy]benzoic acid (390 mg, 700 mmol, 1.0 equiv) and 4-(3-amino-2,2,4,4-tetramethyl-cyclobutoxy)-2-chloro-benzonitrile (275 mg, 700 mmol, 1.0 equiv, TFA salt) in DMF (3 mL) were added DIEA (362 mg, 2.80 mmol, 4.0 equiv) and HATU (319 mg, 839 mmol, 1.2 equiv). The mixture was stirred at 30° C. for 1 h. The solution was purified by prep-HPLC (column: Phenomenex luna C18 150*40 mm* 15 μm; mobile phase: [water (0.1% TFA)-ACN]; B %: 52%-82%, 11 min) to give tert-butyl-N-[2-[2-[2-[2-[2-[4-[[3-(3-chloro-4-cyano-phenoxy)-2,2,4,4-tetramethyl-cyclobutyl]-carbamoyl]phenoxy]ethoxy]ethoxy]ethoxy]ethoxy]ethyl]carbamate (340 mg, 459 mmol, 66% yield, 97% purity) as a colorless gum. LC-MS: MS (ES⁺): RT=1.070 min, m/z=718.3 [M+H⁺].

### 33.7 Preparation of Compound 9

Prepared similarly to compound 4 in Compound I-24. The mixture was concentrated to give 4-[2-[2-[2-[2-(2-aminoethoxy)ethoxy]ethoxy]ethoxy]ethoxy]-N-[3-(3-chloro-4-cyano-phenoxy)-2,2,4,4-tetramethyl-cyclobutyl]benzamide (91 mg, crude, TFA salt) as a colorless gum. LC-MS: MS (ES⁺): RT=0.638 min, m/z=618.2 [M+H⁺].

### 33.8 Preparation of Compound I-33

To a mixture of 4-[2-[2-[2-[2-(2-aminoethoxy)ethoxy]ethoxy]ethoxy]ethoxy]-N-[3-(3-chloro-4-cyano-phenoxy)-2,2,4,4-tetramethyl-cyclobutyl]benzamide (91 mg, 0.12 mmol, 1.0 equiv, TFA salt) and 2-[(9S)-7-(4-chlorophenyl)-4,5,13-trimethyl-3-thia-1,8,11,12-tetrazatricyclo[8.3.0.0^{2,6}]trideca-2 (6),4,7, 10, 12-pentaen-9-yl]acetic acid (50 mg, 0.12 mmol, 1.0 equiv) in DMF (1.5 mL) were added DIEA (64 mg, 0.50 mmol, 4.0 equiv) and HATU (56 mg, 0.15 mmol, 1.2 equiv). The mixture was stirred at 30° C. for 1 h. The mixture was purified by prep-HPLC (column: Unisil 3-100 C18 Ultra 150*50 mm*3 μm; mobile phase: [water (0.225% FA)-ACN]; B %: 55%-85%, 10 min) to give N-[3-(3-chloro-4-cyano-phenoxy)-2,2,4,4-tetramethyl-cyclobutyl]-4-[2-[2-[2-[2-[2-[[2-[(9S)-7-(4-chlorophenyl)-4,5, 13-trimethyl-3-thia-1,8, 11,12-tetrazatricyclo[8.3.0.0^{2,6}]trideca-2 (6),4,7, 10,12-pentaen-9-yl]acetyl]amino]ethoxy]ethoxy]ethoxy]ethoxy]ethoxy]benzamide (58 mg, 58 mmol, 47% yield, 100% purity) as an off-white solid. LC-MS: MS (ES⁺): RT=2.593 min, m/z=1001.3 [M+H⁺]; LC-MS method 40. 1H NMR (400 MHZ, MeOD): δ 8.44 (t, J=4.8 Hz, 1H), 7.78 (d, J=8.8 Hz, 2H), 7.72 (d, J=8.4 Hz, 1H), 7.63 (d, J=8.8 Hz, 1H), 7.51-7.36 (m, 4H), 7.12 (d, J=2.4 Hz, 1H), 7.05-6.93 (m, 3H), 4.69-4.56 (m, 1H), 4.28 (s, 1H), 4.22-4.11 (m, 3H), 3.87-3.81 (m, 2H), 3.70-3.58 (m, 14H), 3.50-3.41 (m, 3H), 3.30-3.25 (m, 1H), 2.68 (s, 3H), 2.44 (s, 3H), 1.69 (s, 3H), 1.27 (s, 6H), 1.22 (s, 6H).

### Example 34—Synthesis of Compound I-34

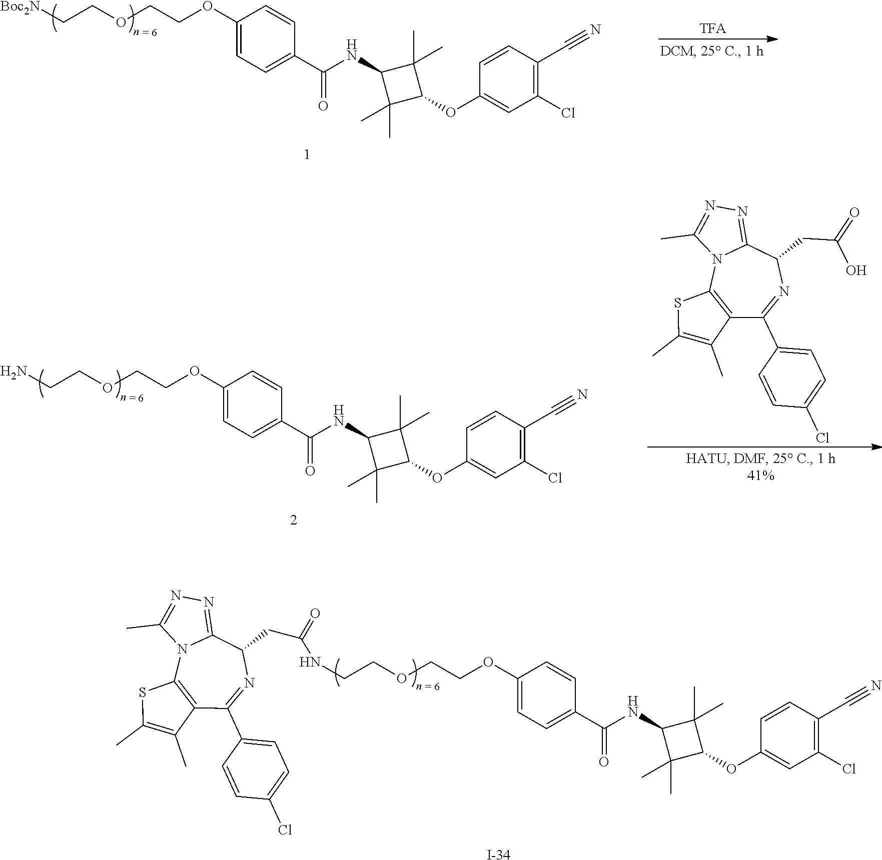

### 34.1 Preparation of Compound 2

Prepared similarly to compound 4 in Compound I-24. The mixture was concentrated to give crude 4-[2-[2-[2-[2-[2-[2-(2-aminoethoxy)ethoxy]ethoxy]ethoxy]ethoxy]ethoxy]ethoxy]-N-[3-(3-chloro-4-cyano-phenoxy)-2,2,4,4-tetramethyl-cyclobutyl]benzamide (70 mg, crude) as a colorless gum. LC-MS: MS (ES⁺): RT=1.077 min, m/z=706.2 [M+H⁺].

### 34.2 Preparation of Compound I-34

To a mixture of 4-[2-[2-[2-[2-[2-[2-(2-aminoethoxy)ethoxy]ethoxy]ethoxy]ethoxy]ethoxy]ethoxy]-N-[3-(3-chloro-4-cyano-phenoxy)-2,2,4,4-tetramethyl-cyclobutyl]benzamide (70 mg, 99 mmol, 1.0 equiv) and 2-[(9S)-7-(4-chlorophenyl)-4,5,13-trimethyl-3-thia-1,8,11,12-tetrazatricyclo[8.3.0.0^{2,6}]trideca-2 (6),4,7,10,12-pentaen-9-yl]acetic acid (39 mg, 99 mmol, 1.0 equiv) in DMF (1.5 mL) were added DIEA (51 mg, 0.40 mmol, 4.0 equiv) and HATU (45 mg, 0.12 mmol, 1.2 equiv). The mixture was stirred at 25° C. for 1 h. The mixture was purified by prep-HPLC (column: Unisil 3-100 C18 Ultra 150*50 mm*3 μm; mobile phase: [water (0.225% FA)-ACN]; B %: 57%-87%, 10 min) to give N-[3-(3-chloro-4-cyano-phenoxy)-2,2,4,4-tetramethyl-cyclobutyl]-4-[2-[2-[2-[2-[2-[2-[2-[2-[(9S)-7-(4-chlorophenyl)-4,5,13-trimethyl-3-thia-1,8,11,12-tetrazatricyclo[8.3.0.0^{2,6}]trideca-2 (6),4,7,10,12-pentaen-9-yl]acetyl]amino]ethoxy]ethoxy]ethoxy]ethoxy]ethoxy]ethoxy]ethoxy]benzamide (44 mg, 41 mmol, 41% yield, 100% purity) as a white solid. LC-MS: MS (ES⁺): RT=2.574 min, m/z=1090.8 [M+H⁺]; LC-MS method 40. 1H NMR (400 MHZ, MeOD): δ 8.52-8.39 (m, 1H), 7.80 (d, J=8.8 Hz, 2H), 7.71 (d, J=8.8 Hz, 1H), 7.64 (d, J=8.8 Hz, 1H), 7.50-7.36 (m, 4H), 7.12 (d, J=2.4 Hz, 1H), 7.05-6.92 (m, 3H), 4.67-4.57 (m, 1H), 4.30-4.26 (m, 1H), 4.21-4.12 (m, 3H), 3.88-3.82 (m, 2H), 3.70-3.67 (m, 2H), 3.66-3.59 (m, 20H), 3.50-3.42 (m, 3H), 3.30-3.25 (m, 1H), 2.69 (s, 3H), 2.44 (s, 3H), 1.69 (s, 3H), 1.28 (s, 6H), 1.22 (s, 6H).

### Example 35—Synthesis of Compound I-35

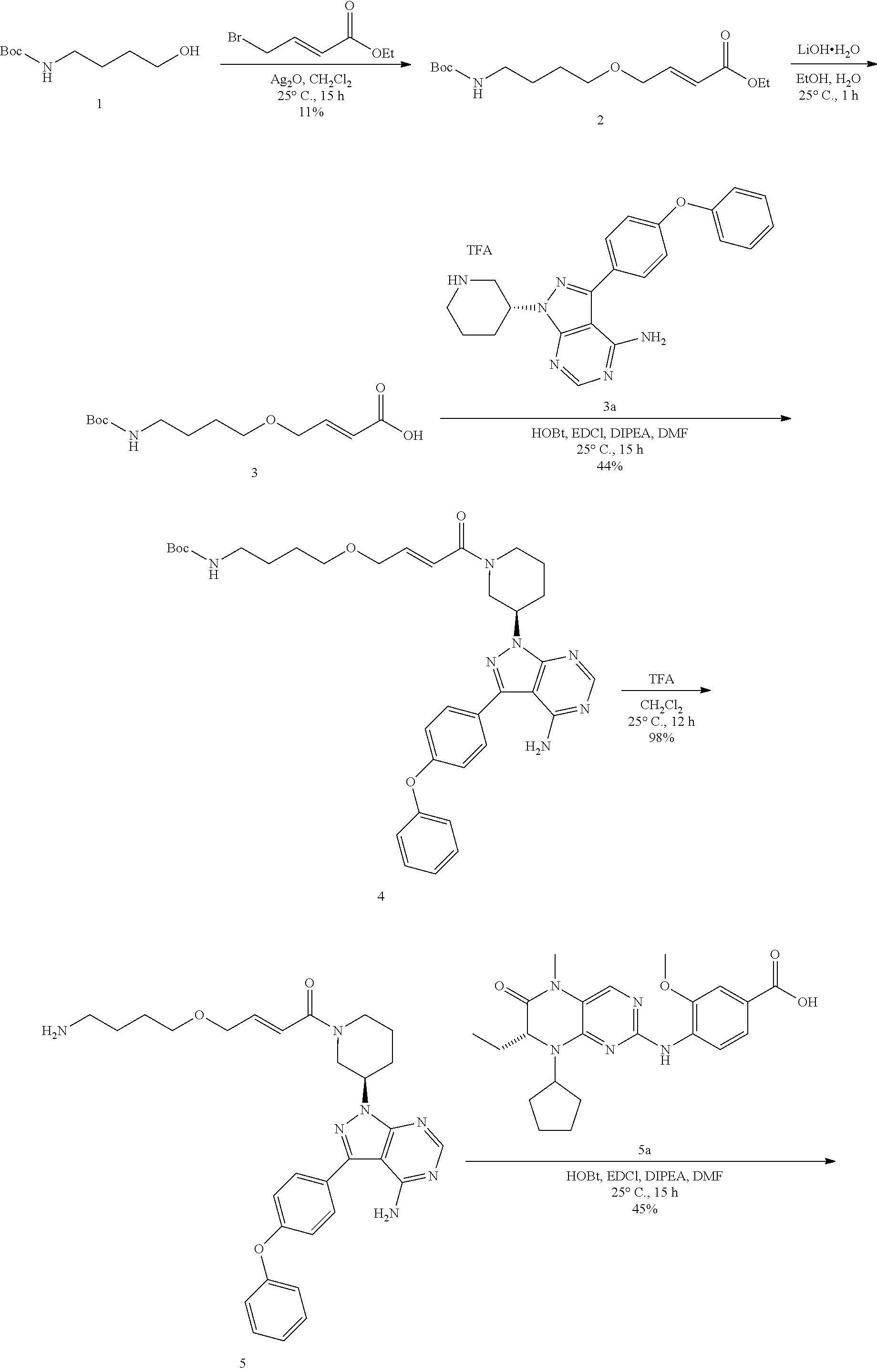

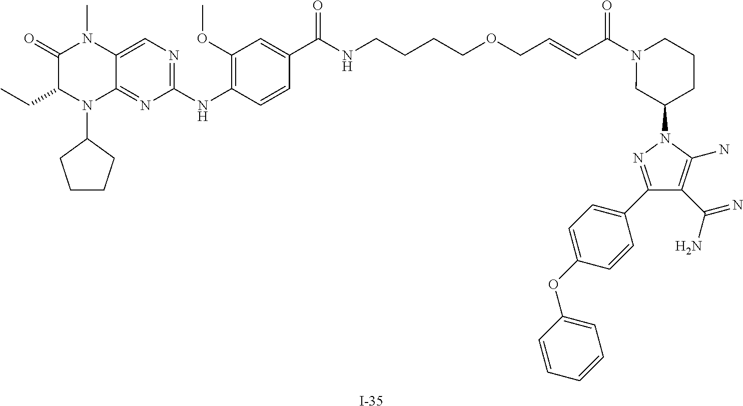

### 35.1 Preparation of Compound 2

To a solution o ethyl (E)-4-bromobut-2-enoate (4.08 g, 21.1 mmol, 2.91 mL, 2.0 equiv) and tert-butyl N-(4-hydroxybutyl) carbamate (2.00 g, 10.6 mmol, 1.0 equiv) in CH₂Cl₂ (1 mL) was added Ag₂O (2.45 g, 10.6 mmol, 1.0 equiv) at 25° C. under N₂. The mixture was stirred at 25° C. for 15 h. The reaction mixture was filtered and the filtrate was concentrated in vacuo. The residue was purified by column chromatography on silica gel (petroleum ether: ethyl acetate=100:1 to 1:1) to afford ethyl (E)-4-[4-(tert-butoxycarbonylamino)butoxy]but-2-enoate (0.37 g, 1.21 mmol, 11% yield) as a yellow oil. LC-MS: MS (ES⁺): RT=0.914 min, m/z=202.1 [M-100+H⁺]. ¹H NMR (400 MHZ, CDCl₃): δ 7.07-6.88 (m, 1H), 6.12-5.99 (m, 1H), 4.76-4.45 (m, 1H), 4.28-4.19 (m, 2H), 4.17-4.06 (m, 2H), 3.60-3.42 (m, 2H), 3.26-3.07 (m, 2H), 1.65-1.55 (m, 4H), 1.44 (s, 9H), 1.29 (t, 3H, J=7.2 Hz).

### 35.2 Preparation of Compound 3

To a solution of ethyl (E)-4-[4-(tert-butoxycarbonylamino)butoxy]but-2-enoate (80.0 mg, 0.27 mmol, 1.0 equiv) ethyl (E)-4-[4-(tert-butoxycarbonylamino)butoxy]but-2-enoate (80.0 mg, 0.27 mmol, 1.0 equiv) in EtOH (1 mL) and H₂O (1 mL) was added LiOH·H₂O (33.4 mg, 0.80 mmol, 3.0 equiv). The mixture was stirred at 25° C. for 1 h. The reaction mixture was concentrated in vacuo and the residue was diluted with H₂O (10 mL). The pH of the mixture was adjusted to 7 by addition of 1 N aq. HCl and the mixture was concentrated in vacuo to afford (E)-4-[4-(tert-butoxycarbonylamino)butoxy]but-2-enoic acid (72.0 mg, crude) as a yellow solid. LC-MS: MS (ES⁺): RT=0.814 min, m/z=174.2 [M-100+H⁺]. ¹H NMR (400 MHz, DMSO-d₆) δ 6.86-6.68 (m, 1H), 6.34-6.15 (m, 1H), 5.87-5.65 (m, 1H), 3.95-3.91 (m, 2H), 2.93-2.88 (m, 2H), 1.52-1.37 (m, 6H), 1.37-1.35 (s, 9H).

### 35.3 Preparation of Compound 4

To a solution of 3-(4-phenoxyphenyl)-1-[(3R)-3-piperidyl]pyrazolo[3,4-d]pyrimidin-4-amine (127 mg, 0.18 mmol, 69% purity, 1.0 equiv, TFA salt) and (E)-4-[4-(tert-butoxycarbonylamino)butoxy]but-2-enoic acid (72.0 mg, 0.26 mmol, 1.5 equiv) in DMF (1 mL) were added EDCI (67.3 mg, 0.35 mmol, 2.0 equiv) and HOBt (47.5 mg, 0.35 mmol, 2.0 equiv) and DIPEA (182 mg, 1.40 mmol, 0.25 mL, 8.0 equiv) at 25° C. under N₂. The mixture was stirred at 25° C. and stirred for 15 h. The reaction mixture was diluted with water (20 mL) and the mixture was extracted with ethyl acetate (15 mL*3). The combined organic phase was washed with brine (10 mL), dried with anhydrous Na₂SO₄, filtered and concentrated in vacuo. The residue was purified by prep-TLC on silica gel (Dichloromethane: Methanol=10:1) to afford tert-butyl N-[4-[(E)-4-[(3R)-3-[4-amino-3-(4-phenoxyphenyl) pyrazolo[3,4-d]pyrimidin-1-yl]-1-piperidyl]-4-oxo-but-2-enoxy]butyl]carbamate (50.0 mg, 0.08 mmol, 44% yield) as a yellow oil. LC-MS: MS (ES⁺): RT=0.744 min, m/z=642.3 [M+H⁺]. ¹H NMR (400 MHZ, CDCl₃): δ 8.36 (s, 1H), 7.69-7.61 (m, 2H), 7.43-7.35 (m, 2H,), 7.21-7.13 (m, 3H), 7.13-7.04 (m, 2H), 6.98-6.71 (m, 1H), 6.54-6.35 (m, 1H), 5.78-5.44 (m, 2H), 5.00-4.73 (m, 2H), 4.66-4.42 (m, 1H), 4.14-4.04 (m, 2H), 3.90-3.65 (m, 1H), 3.55-3.41 (m, 2H), 3.25-3.11 (m, 2H), 2.55-2.24 (m, 2H), 1.87-1.70 (m, 4H), 1.-1.52 (m, 2H), 1.43 (s, 9H).

### 35.4 Preparation of Compound 5

A mixture of tert-butyl N-[4-[(E)-4-[(3R)-3-[4-amino-3-(4-phenoxyphenyl) pyrazolo[3,4-d]pyrimidin-1-yl]-1-piperidyl]-4-oxo-but-2-enoxy]butyl]carbamate (40.0 mg, 0.06 mmol, 1.0 equiv) in DCM (1 mL) and TFA (0.5 mL) was stirred at 25° C. for 1 h. The reaction mixture was concentrated in vacuo to afford (E)-4-(4-aminobutoxy)-1-[(3R)-3-[4-amino-3-(4-phenoxyphenyl) pyrazolo[3,4-d]pyrimidin-1-yl]-1-piperidyl]but-2-en-1-one (40.0 mg, 0.06 mmol, 98% yield, TFA salt) as a yellow oil. LC-MS: MS (ES⁺): RT=0.598 min, m/z=542.2 [M+H⁺].

### 35.5 Preparation of Compound I-35

To a solution of (E)-4-(4-aminobutoxy)-1-[(3R)-3-[4-amino-3-(4-phenoxyphenyl) pyrazolo[3,4-d]pyrimidin-1-yl]-1-piperidyl]but-2-en-1-one (40.0 mg, 0.06 mmol, 1.0 equiv, TFA salt) and 4-[(8-cyclopentyl-7-ethyl-5-methyl-6-oxo-7H-pteridin-2-yl)amino]-3-methoxy-benzoic acid (26.0 mg, 0.06 mmol, 1.0 equiv) in DMF (1 mL) were added EDCI (29.2 mg, 0.15 mmol, 2.5 equiv) and HOBt (20.6 mg, 0.15 mmol, 2.5 equiv) and DIPEA (78.9 mg, 0.61 mmol, 0.11 mL, 10.0 equiv) at 25° C. under N₂. The mixture was stirred at 25° C. for 14 h. The reaction mixture was diluted with water (20 mL) and the mixture was extracted with ethyl acetate (15 mL*3). The combined organic phase was washed with brine (10 mL), dried with anhydrous Na₂SO₄, filtered and concentrated in vacuo. The residue was purified by prep-HPLC (column: Waters Xbridge 150*25 mm*5 μm; mobile phase: [water (10 mM NH₄HCO₃)-ACN]; B %: 55%-85%, 10 min) to afford N-[4-[(E)-4-[(3R)-3-[4-amino-3-(4-phenoxyphenyl) pyrazolo[3,4-d]pyrimidin-1-yl]-1-piperidyl]-4-oxo-but-2-enoxy]butyl]-4-[(8-cyclopentyl-7-ethyl-5-methyl-6-oxo-7H-pteridin-2-yl)amino]-3-methoxy-benzamide (26.9 mg, 0.03 mmol, 45% yield, 98% purity) as an off-white solid. LC-MS: MS (ES⁺): RT=2.171 min, m/z=950.4 [M+H⁺]; LC-MS Method 25. 1H NMR (400 MHZ, DMSO-d₆): δ 8.43-8.24 (m, 3H), 7.84 (s, 1H), 7.71-7.56 (m, 3H), 7.51-7.40 (m, 4H), 7.20-7.10 (m, 5H), 6.70-6.42 (m, 2H), 4.73-3.69 (m, 11H), 3.44 (s, 3H), 3.27-3.06 (m, 7H), 2.28-2.20 (m, 1H), 2.16-2.09 (m, 1H), 2.04-1.87 (m, 4H), 1.81-1.71 (m, 4H), 1.65-1.48 (m, 8H), 0.75 (t, 3H, J=7.6 Hz).

### Example 36—Synthesis of Compound I-36

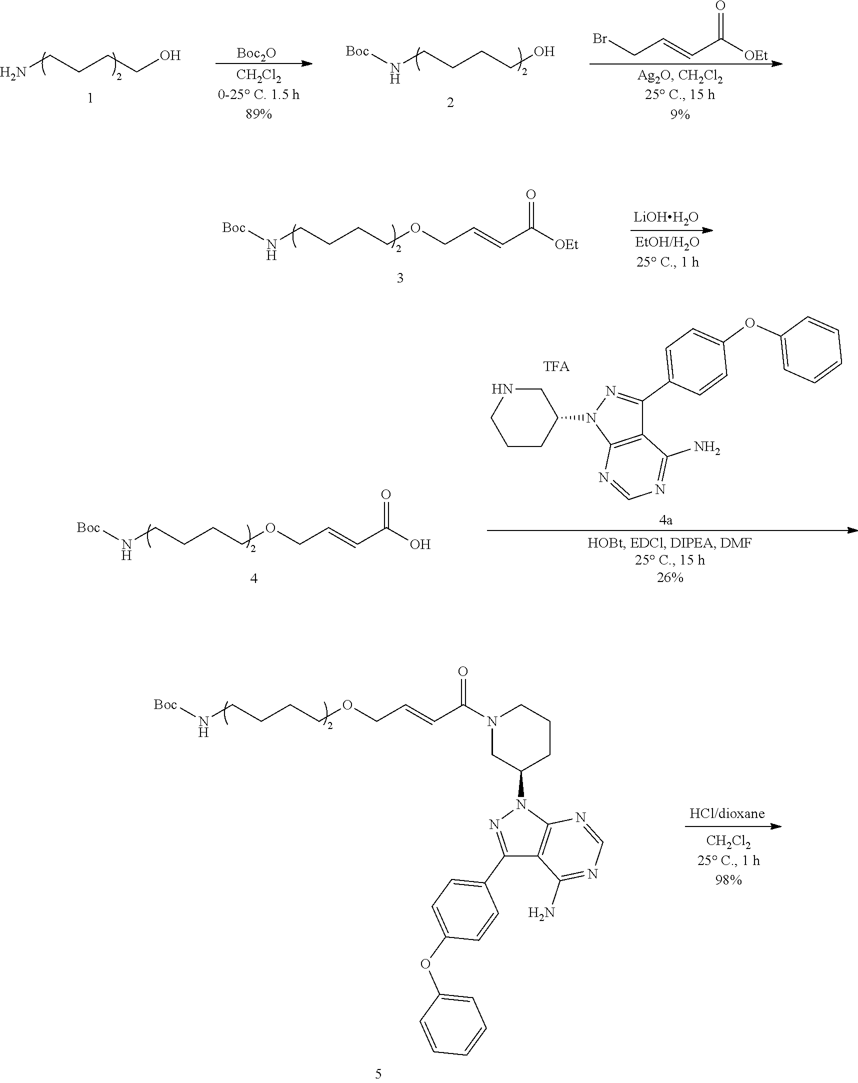

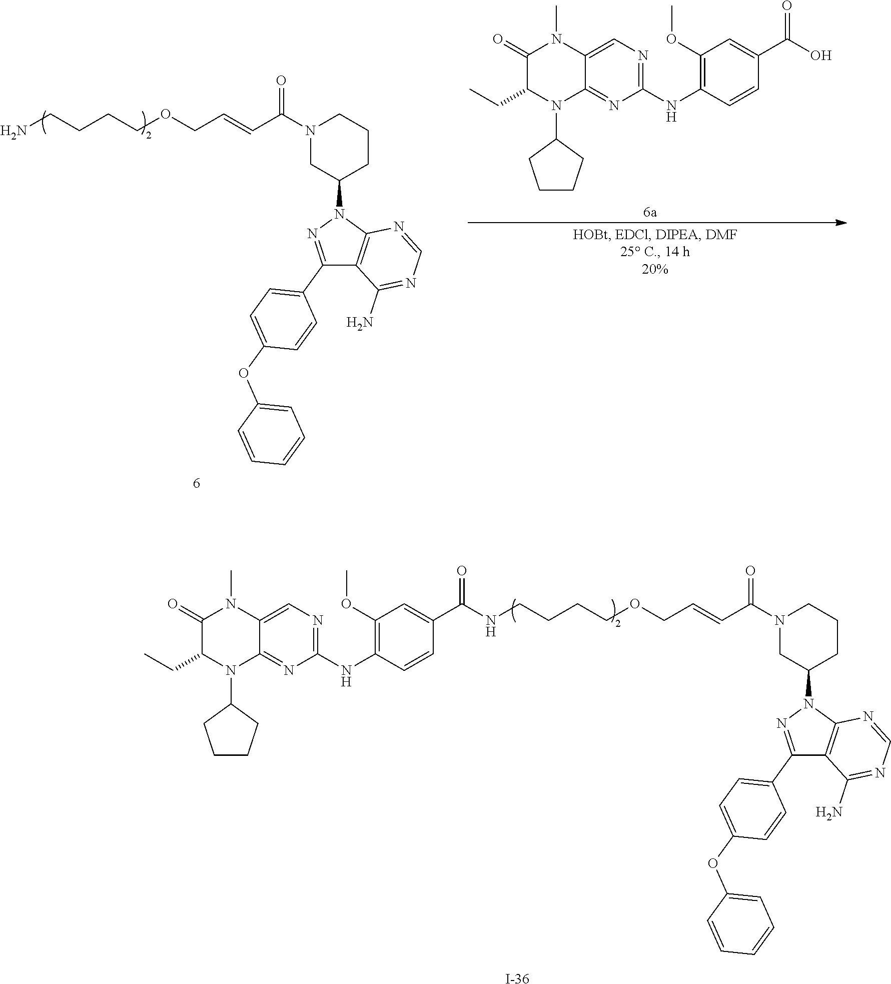

### 36.1 Preparation of Compound 2

To a solution of 8-aminooctan-1-ol (5.0 g, 34.4 mmol, 1.0 equiv) in DCM (50 mL) was added Boc₂O (9.02 g, 41.3 mmol, 9.49 mL, 1.2 equiv) at 0° C. The reaction mixture was stirred at 25° C. for 1.5 h. The reaction mixture was concentrated in vacuo. The residue was purified by column chromatography on silica gel (petroleum ether: ethyl acetate=100:1 to 5:1) to afford tert-butyl N-(8-hydroxyoctyl) carbamate (7.54 g, 30.7 mmol, 89% yield) as a white solid. ¹H NMR (400 MHZ, CDCl₃): δ 4.76-4.30 (m, 1H), 3.63 (t, 2H, J=6.4 Hz), 3.24-3.01 (m, 2H), 1.64-1.47 (m, 4H), 1.44 (s, 9H), 1.38-1.27 (m, 8H).

### 36.2 Preparation of Compound 3

To a solution of ethyl (E)-4-bromobut-2-enoate (3.15 g, 16.3 mmol, 2.25 mL, 2.0 equiv) and tert-butyl N-(8-hydroxyoctyl) carbamate (2.0 g, 8.15 mmol, 1.0 equiv) in CH₂Cl₂ (1 mL) was added Ag₂O (1.89 g, 8.15 mmol, 1.0 equiv) at 25° C. under N₂. The mixture was stirred at 25° C. for 15 h. The reaction mixture was filtered and the filtrate was concentrated in vacuo. The residue was purified by column chromatography on silica gel (petroleum ether: ethyl acetate=100:1 to 1:1) to afford ethyl (E)-4-[8-(tert-butoxycarbonylamino)octoxy]but-2-enoate (260 mg, 0.73 mmol, 9% yield) as a colorless oil. LC-MS: MS (ES⁺): RT=1.029 min, m/z=258.3 [M-100+H⁺]. ¹H NMR (400 MHZ, CDCl₃): δ 7.05-6.90 (m, 1H), 6.13-6.01 (m, 1H), 4.55-4.37 (m, 1H), 4.23-4.17 (m, 2H), 4.13-4.11 (m, 2H), 3.50-3.40 (m, 2H), 3.14-3.20 (m, 2H), 2.07-2.01 (m, 2H), 1.65-1.55 (m, 2H), 1.44 (s, 9H), 1.32-1.28 (m, 8H), 1.28-1.23 (m, 3H).

### 36.3 Preparation of Compound 4

To a solution of ethyl (E)-4-[8-(tert-butoxycarbonylamino)octoxy]but-2-enoate (130 mg, 0.36 mmol, 1.0 equiv) ethyl (E)-4-[8-(tert-butoxycarbonylamino)octoxy]but-2-enoate (130 mg, 0.36 mmol, 1.0 equiv) in EtOH (1 mL) and H₂O (1 mL) was added LiOH·H₂O (45.8 mg, 1.09 mmol, 3.0 equiv) at 25° C. The mixture was stirred at 25° C. for 1 h. The reaction mixture was concentrated in vacuo and the residue was diluted with H₂O (5 mL). The pH of the mixture was adjusted to 7 by addition of 1 N aq. HCl and the mixture was concentrated in vacuo to afford (E)-4-[8-(tert-butoxycarbonylamino)octoxy]but-2-enoic acid (119 mg, 0.36 mmol, crude) as a white solid. LC-MS: MS (ES⁺): RT=0.931 min, m/z=230.3 [M-100+H⁺].

### 36.4 Preparation of Compound 5

To a solution of 3-(4-phenoxyphenyl)-1-[(3R)-3-piperidyl]pyrazolo[3,4-d]pyrimidin-4-amine (153 mg, 0.36 mmol, 1.0 equiv, HCl salt) and (E)-4-[8-(tert-butoxycarbonylamino) octoxy]but-2-enoic acid (119 mg, 0.36 mmol, 1.0 equiv) in DMF (2 mL) were added EDCI (104 mg, 0.54 mmol, 1.5 equiv) and HOBt (73.3 mg, 0.54 mmol, 1.5 equiv) and DIPEA (187 mg, 1.45 mmol, 0.25 mL, 4.0 equiv) at 25° C. under N₂. The mixture was stirred at 25° C. for 15 h. The reaction mixture was diluted with water (20 mL) and the mixture was extracted with ethyl acetate (15 mL*3). The combined organic phase was washed with brine (10 mL), dried with anhydrous Na₂SO₄, filtered and concentrated in vacuo. The residue was purified by prep-HPLC (column: Phenomenex luna C18 150*25 mm*10 μm; mobile phase: [water (0.225% FA)-ACN]; B %: 63%-93%,10 min) to afford tert-butyl N-[8-[(E)-4-[(3R)-3-[4-amino-3-(4-phenoxyphenyl) pyrazolo[3,4-d]pyrimidin-1-yl]-1-piperidyl]-4-oxo-but-2-enoxy]octyl]carbamate (65 mg, 0.09 mmol, 26% yield) as a colorless oil. LC-MS: MS (ES⁺): RT=0.810 min, m/z=698.3 [M+H⁺]. ¹H NMR (400 MHZ, DMSO-d₆): δ 8.25 (s, 1H), 7.72-7.61 (m, 2H), 7.48-7.36 (m, 2H), 7.23-7.07 (m, 5H), 6.80-6.40 (m, 3H), 4.77-4.53 (m, 1H), 4.38-3.63 (m, 4H), 3.30-3.28 (m, 2H), 2.94-2.80 (m, 2H), 2.29-1.78 (m, 4H), 1.66-1.09 (m, 23H).

### 36.5 Preparation of 6

A mixture of tert-butyl N-[8-[(E)-4-[(3R)-3-[4-amino-3-(4-phenoxyphenyl) pyrazolo[3,4-d]pyrimidin-1-yl]-1-piperidyl]-4-oxo-but-2-enoxy]octyl]carbamate (55.0 mg, 0.08 mmol, 1.0 equiv) in CH₂Cl₂ (0.5 mL) and 4 M HCl/dioxane (0.5 mL) stirred at 25° C. for 1 h. The reaction mixture was concentrated in vacuo to afford (E)-4-(8-aminooctoxy)-1-[(3R)-3-[4-amino-3-(4-phenoxyphenyl) pyrazolo[3,4-d]pyrimidin-1-yl]-1-piperidyl]but-2-en-1-one (52.0 mg, 0.08 mmol, 98% yield, 2HCl salt) as a white solid. LC-MS: MS (ES⁺): RT=0.830 min, m/z=598.5 [M+H⁺].

### 36.6 Preparation of Compound I-36

To a solution of 4-[(8-cyclopentyl-7-ethyl-5-methyl-6-oxo-7H-pteridin-2-yl)amino]-3-methoxy-benzoic acid (33.0 mg, 0.08 mmol, 1.0 equiv) and (E)-4-(8-aminooctoxy)-1-[(3R)-3-[4-amino-3-(4-phenoxyphenyl) pyrazolo[3,4-d]pyrimidin-1-yl]-1-piperidyl]but-2-en-1-one (52.0 mg, 0.08 mmol, 1.0 equiv, 2HCl salt) in DMF (1 mL) were added EDCI (37.2 mg, 0.19 mmol, 2.5 equiv) and HOBt (26.2 mg, 0.19 mmol, 2.5 equiv) and DIPEA (80.2 mg, 0.62 mmol, 0.11 mL, 8.0 equiv) at 25° C. under N₂. The mixture was stirred at 25° C. for 14 h. The reaction mixture was diluted with water (20 mL) and the mixture was extracted with ethyl acetate (15 mL*3). The combined organic phase was washed with brine (10 mL), dried with anhydrous Na₂SO₄, filtered and concentrated in vacuo. The residue was purified by prep-HPLC (column: Waters Xbridge 150*25 mm*5 μm; mobile phase: [water (10 mM NH₄HCO₃)-ACN]; B %: 62%-92%, 10 min) to afford N-[8-[(E)-4-[(3R)-3-[4-amino-3-(4-phenoxyphenyl) pyrazolo[3,4-d]pyrimidin-1-yl]-1-piperidyl]-4-oxo-but-2-enoxy]octyl]-4-[[(7R)-8-cyclopentyl-7-ethyl-5-methyl-6-oxo-7H-pteridin-2-yl]amino]-3-methoxy-benzamide (15.8 mg, 0.02 mmol, 20% yield, 98% purity) as a yellow solid. LC-MS: MS (ES⁺): RT=1.952 min, m/z=1005.5 [M+H⁺]; LC-MS Method 40. 1H NMR (400 MHZ, DMSO-d₆): δ 8.42-8.37 (m, 1H), 8.33-8.20 (m, 2H), 7.84 (s, 1H), 7.69-7.55 (m, 3H), 7.50-7.40 (m, 4H), 7.21-7.09 (m, 5H), 6.69-6.46 (m, 2H), 4.78-4.50 (m, 2H), 4.37-4.30 (m, 1H), 4.25-4.21 (m, 1H), 4.14-4.06 (m, 2H), 3.98-3.94 (m, 1H), 3.92 (s, 3H), 3.78-3.65 (m, 1H), 3.51-3.38 (m, 2H), 3.25-3.23 (m, 5H), 2.12-1.71 (m, 10H), 1.64-1.43 (m, 8H), 1.36-1.19 (m, 9H), 0.76 (t, 3H, J=7.2 Hz).

### Example 37—Synthesis of Compound I-37

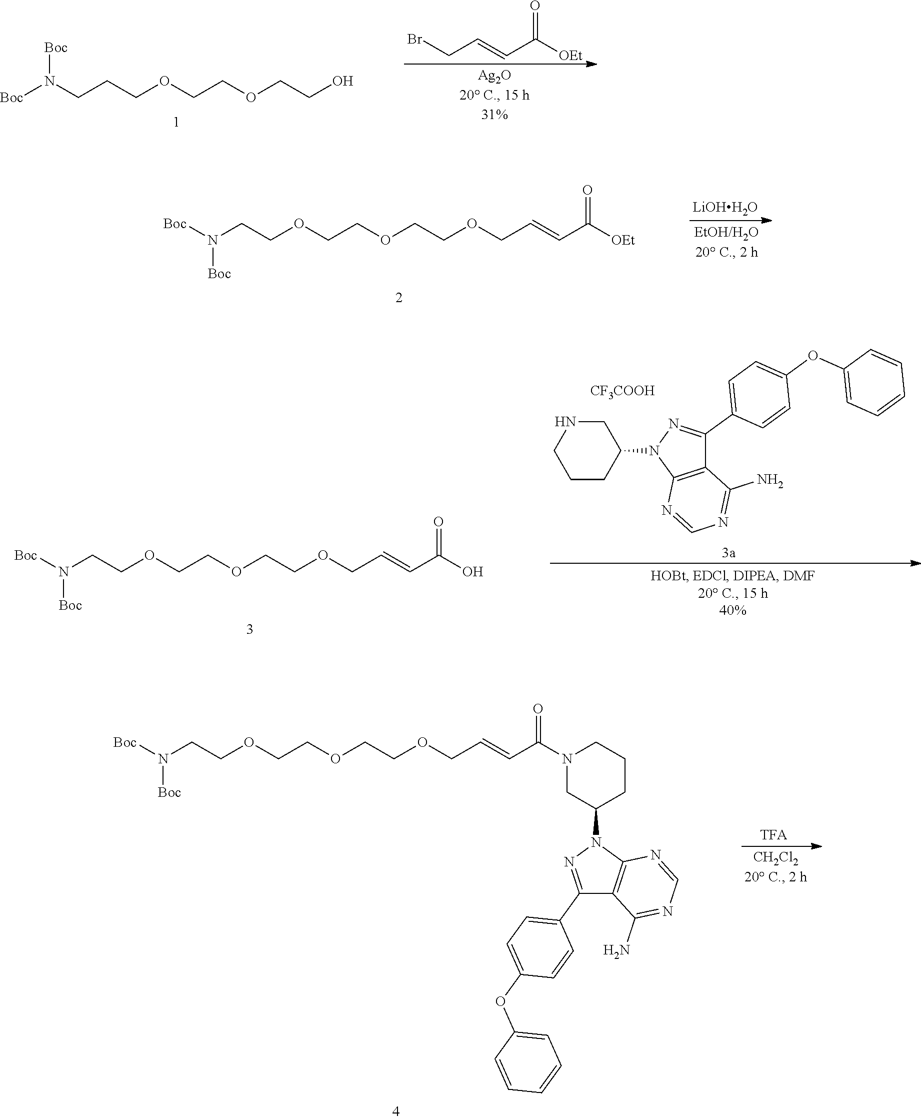

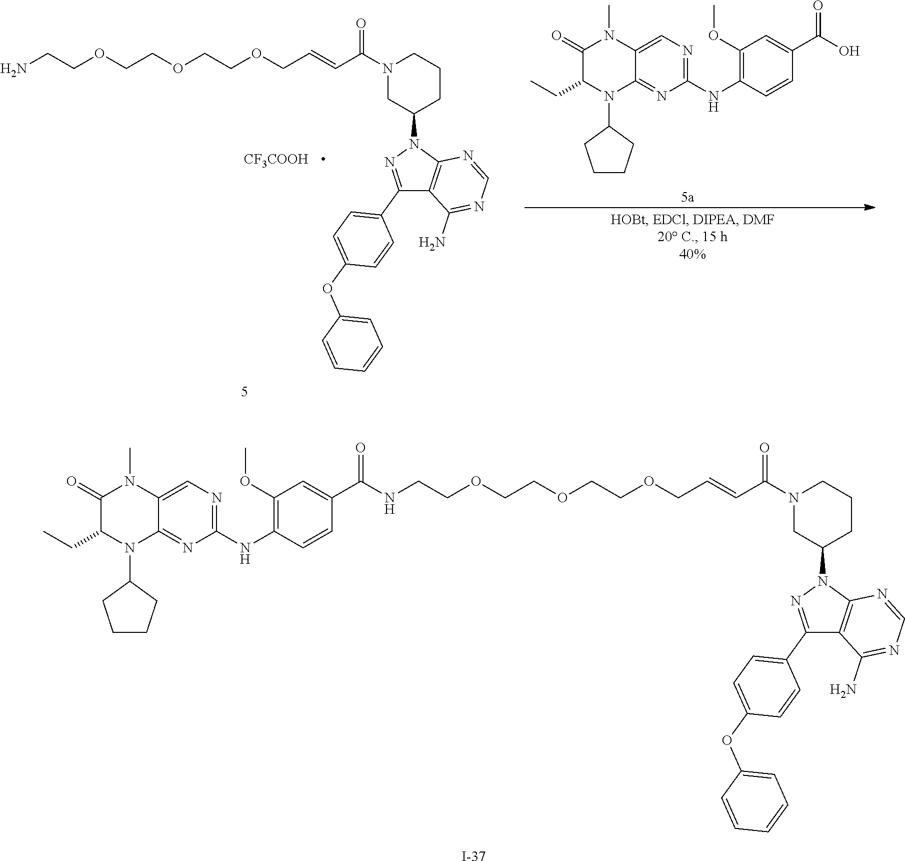

### 37.1 Preparation of Compound 2

A mixture of tert-butyl N-tert-butoxycarbonyl-N-[2-[2-(2-hydroxyethoxy) ethoxy]ethyl]carbamate (500 mg, 1.43 mmol, 1.0 equiv), ethyl (E)-4-bromobut-2-enoate (552 mg, 2.86 mmol, 2.0 equiv) and Ag₂O (332 mg, 1.43 mmol, 1.0 equiv) was stirred at 20° C. for 15 h under N₂. The reaction mixture was diluted with DCM (10 mL). The mixture was filtered and the filtrate was concentrated in vacuo. The residue was purified by column chromatography on silica gel (petroleum ether/ethyl acetate=10/1 to 1/1) to afford ethyl (E)-4-[2-[2-[2-[bis(tert-butoxycarbonyl)amino]ethoxy]ethoxy]ethoxy]but-2-enoate (230 mg, 0.44 mmol, 31% yield) as a yellow oil. ¹H NMR (400 MHZ, CDCl₃): δ 6.99-6.91 (m, 1H), 6.12-6.04 (m, 1H), 4.24-4.17 (m, 4H), 3.82-3.77 (m, 2H), 3.68-3.60 (m, 10H), 1.51 (s, 18H), 1.30 (t, 3H, J=7.2 Hz). LC-MS: MS (ES⁺): RT=0.997 min, m/z=262.1 [M+H⁺-200].

### 37.2 Preparation of Compound 3

To a solution of ethyl (E)-4-[2-[2-[2-[bis(tert-butoxycarbonyl)amino]ethoxy]ethoxy]ethoxy]but-2-enoate (230 mg,0.44 mmol, 1.0 equiv) in EtOH (3 mL) and H₂O (3 mL) was added LiOH·H₂O (55.8 mg, 1.33 mmol, 3.0 equiv). The mixture was stirred at 20° C. for 2 h. The pH of the reaction mixture was adjusted to 6˜7 by addition of 1 N aqueous HCl solution. The mixture was concentrated in vacuo to afford (E)-4-[2-[2-[2-[bis(tert-butoxycarbonyl)amino]ethoxy]ethoxy]ethoxy]but-2-enoic acid (240 mg, crude) as a yellow solid. ¹H NMR (400 MHZ, DMSO-d₆) δ 6.29-6.19 (m, 1H), 5.83-5.75 (m, 1H), 4.00-3.94 (m, 2H), 3.66-3.61 (m, 2H), 3.52-3.45 (m, 10H), 1.43 (s, 18H). LC-MS: MS (ES⁺): RT=0.842 min, m/z=456.1 [M+Na⁺].

37.3 The preparation of compound 4

A mixture of (E)-4-[2-[2-[2-[bis(tert-butoxycarbonyl)amino]ethoxy]ethoxy]ethoxy]but-2-enoic acid (100 mg, 0.23 mmol, 1.0 equiv), 3-(4-phenoxyphenyl)-1-[(3R)-3-piperidyl]pyrazolo[3,4-d]pyrimidin-4-amine (117 mg, 0.16 mmol, 69% purity, 0.7 equiv, TFA salt), EDCI (110 mg, 0.58 mmol, 2.5 equiv), HOBt (77.9 mg, 0.58 mmol, 2.5 equiv) and DIPEA (149 mg, 1.15 mmol, 5.0 equiv) in DMF (3 mL) was stirred at 20° C. for 15 h under N₂. The reaction mixture was quenched with water (10 mL) and extracted with dichloromethane/methanol (10/1, 15 mL*3). The combined organic phase was washed with brine (15 mL), dried over anhydrous Na₂SO₄, filtered and concentrated in vacuo. The residue was purified by prep-TLC on silica gel (dichloromethane/methanol=10/1) to afford tert-butyl N-[2-[2-[2-[(E)-4-[(3R)-3-[4-amino-3-(4-phenoxyphenyl) pyrazolo[3,4-d]pyrimidin-1-yl]-1-piperidyl]-4-oxo-but-2-enoxy]ethoxy]ethoxy]ethyl]-N-tert-butoxycarbonyl-carbamate (75.0 mg, 0.09 mmol, 40% yield) as a colorless oil. ¹H NMR (400 MHZ, CDCl₃) δ 8.38 (brs, 1H), 7.69-7.61 (m, 2H), 7.45-7.37 (m, 2H), 7.21-7.14 (m, 3H), 7.12-7.07 (m, 2H), 6.92-6.77 (m, 1H), 6.64-6.43 (m, 1H), 5.74-5.52 (m, 2H), 4.96-4.80 (m, 1H), 4.27-4.03 (m, 2H), 3.86-3.73 (m, 3H), 3.70-3.53 (m, 12H), 2.45-2.20 (m, 2H), 2.08-1.94 (m, 1H), 1.50 (s, 18H). LC-MS: MS (ES⁺): RT=0.739 min, m/z=802.2 [M+H⁺].

37.4 The preparation of compound 5

A mixture of tert-butyl N-[2-[2-[2-[(E)-4-[(3R)-3-[4-amino-3-(4-phenoxyphenyl) pyrazolo[3,4-d]pyrimidin-1-yl]-1-piperidyl]-4-oxo-but-2-enoxy]ethoxy]ethoxy]ethyl]-N-tert-butoxycarbonyl-carbamate (75.0 mg, 0.09 mmol, 1.0 equiv) in DCM (3 mL) and TFA (1 mL) was stirred at 20° C. for 2 h. The mixture was concentrated in vacuo to afford (E)-4-[2-[2-(2-aminoethoxy)ethoxy]ethoxy]-1-[(3R)-3-[4-amino-3-(4-phenoxyphenyl) pyrazolo[3,4-d]pyrimidin-1-yl]-1-piperidyl]but-2-en-1-one (77 mg, crude, TFA salt) as a yellow oil. LC-MS: MS (ES⁺): RT=0.538 min, m/z=602.1 [M+H⁺].

37.5 The preparation of Compound I-37

A mixture of (E)-4-[2-[2-(2-aminoethoxy)ethoxy]ethoxy]-1-[(3R)-3-[4-amino-3-(4-phenoxyphenyl) pyrazolo[3,4-d]pyrimidin-1-yl]-1-piperidyl]but-2-en-1-one (50.0 mg, 0.07 mmol, 1.0 equiv, TFA salt), 4-[[(7R)-8-cyclopentyl-7-ethyl-5-methyl-6-oxo-7H-pteridin-2-yl]amino]-3-methoxy-benzoic acid (29.7 mg, 0.07 mmol, 1.0 equiv), EDCI (33.5 mg, 0.17 mmol, 2.5 equiv), HOBt (23.6 mg, 0.17 mmol, 2.5 equiv) and DIPEA (45.1 mg, 0.35 mmol, 5.0 equiv) in DMF (2 mL) was stirred at 20° C. for 15 h under N₂. The reaction mixture was quenched by addition water (5 mL) and extracted with dichloromethane/methanol (10/1, 10 mL*3). The combined organic phase was washed with brine (15 mL), dried over anhydrous Na₂SO₄, filtered and the filtrate was concentrated in vacuo. The residue was purified by prep-HPLC (neutral condition: column: Waters Xbridge 150*25 mm*5 μm; mobile phase: [water (10 mM NH₄HCO₃)-ACN]; B %: 52%-82%,9 min) to afford N-[2-[2-[2-[(E)-4-[(3R)-3-[4-amino-3-(4-phenoxyphenyl) pyrazolo[3,4-d]pyrimidin-1-yl]-1-piperidyl]-4-oxo-but-2-enoxy]ethoxy]ethoxy]ethyl]-4-[[(7R)-8-cyclopentyl-7-ethyl-5-methyl-6-oxo-7H-pteridin-2-yl]amino]-3-methoxy-benzamide (29.2 mg, 0.03 mmol, 40% yield, 97% purity) as an off-white solid. ¹H NMR (400 MHZ, DMSO-d₆) δ 8.43-8.34 (m, 2H), 8.25 (s, 1H), 7.83 (s, 1H), 7.70-7.62 (m, 2H), 7.59 (s, 1H), 7.52-7.39 (m, 4H), 7.22-7.09 (m, 5H), 6.71-6.63 (m, 1H), 6.58-6.47 (m, 1H), 4.80-4.61 (m, 1H), 4.40-4.29 (m, 1H), 4.24-4.22 (m, 1H), 4.15-4.07 (m, 2H), 4.05-3.96 (m, 1H), 3.92 (s, 3H), 3.61-3.37 (m, 12H), 3.31-3.27 (m, 2H), 3.24 (s, 3H), 2.29-1.50 (m, 15H), 0.75 (t, 3H, J=7.2 Hz). LC-MS: MS (ES⁺): RT=2.126 min, m/z=1009.4 [M+H⁺]; LC-MS Method 25.

### Example 38—Synthesis of Compound I-38

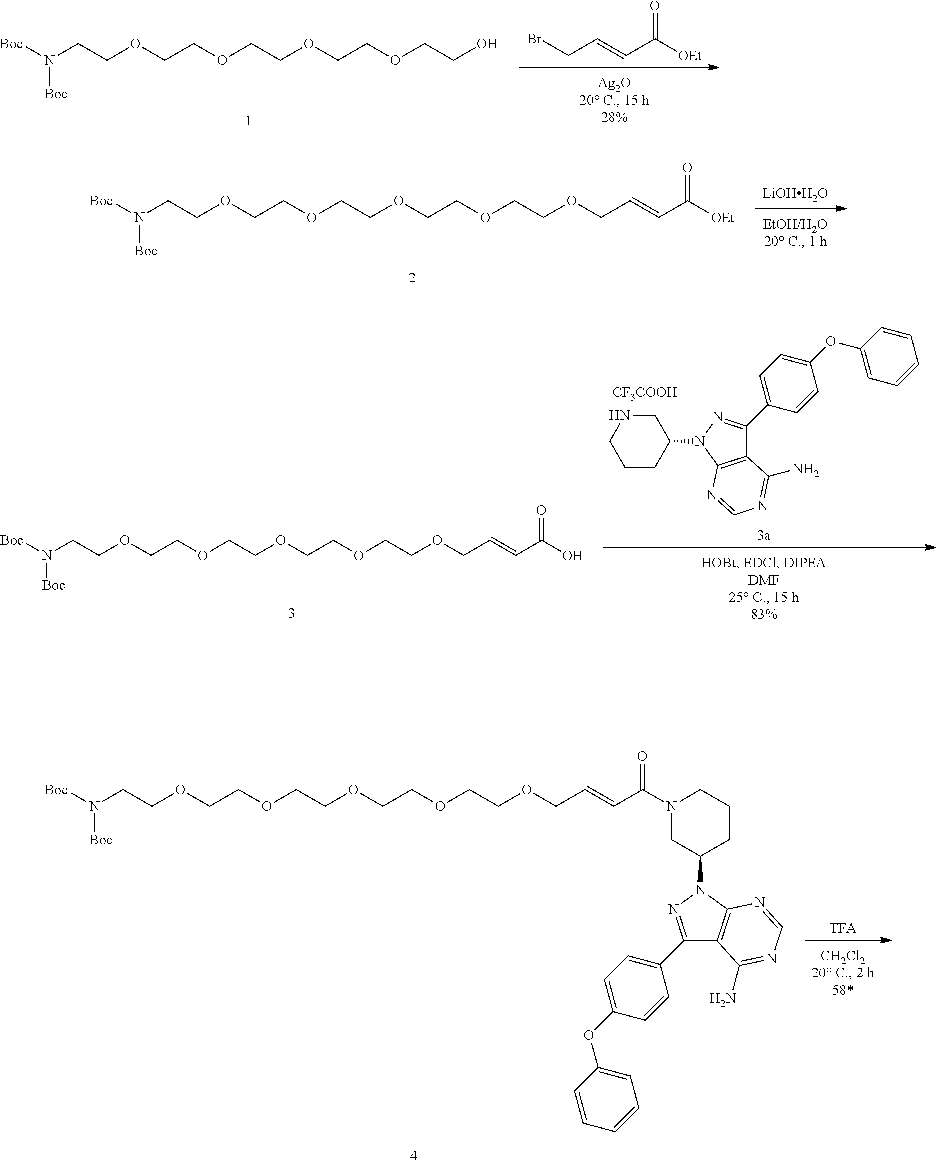

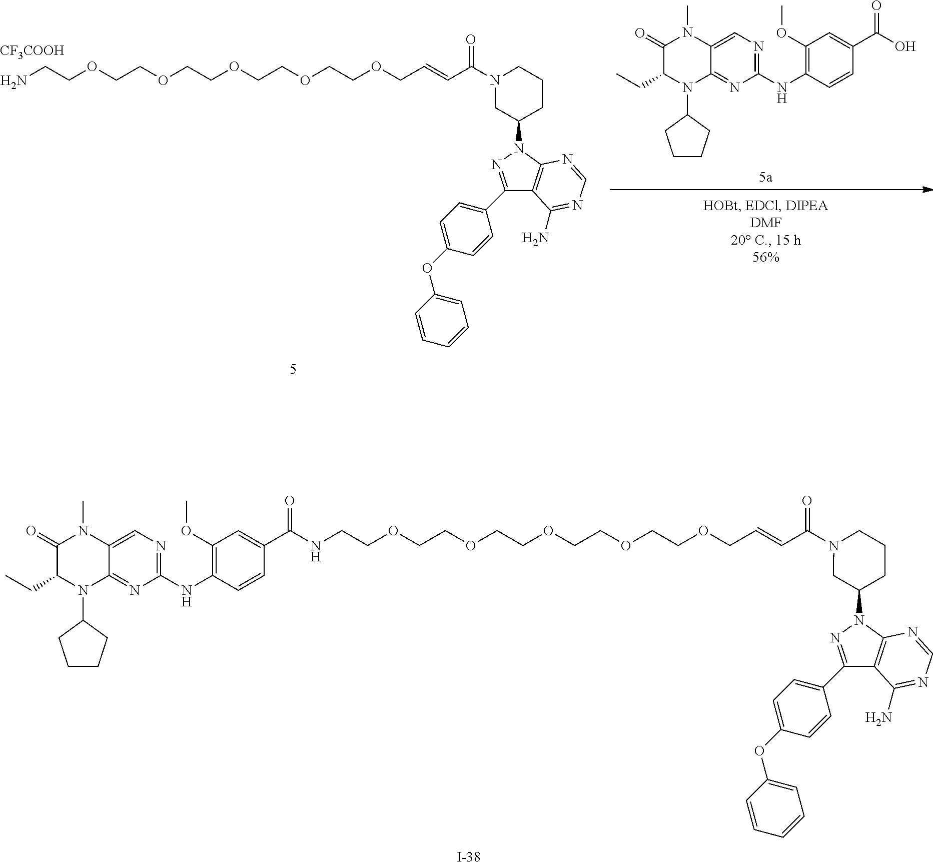

### 38.1 Preparation of Compound 2

A mixture of tert-butyl N-tert-butoxycarbonyl-N-[2-[2-[2-[2-(2-hydroxyethoxy) ethoxy]ethoxy]ethoxy]ethyl]carbamate (500 mg, 1.14 mmol, 1.0 equiv), ethyl (E)-4-bromobut-2-enoate (441 mg, 2.29 mmol, 2.0 equiv), Ag₂O (265 mg, 1.14 mmol, 1.0 equiv) was stirred at 20° C. for 15 h under N₂. The reaction mixture was diluted with DCM (20 mL). The mixture was filtered and the filtrate was concentrated in vacuo. The residue was purified by column chromatography on silica gel (petroleum ether/ethyl acetate=10/1 to 1/1) to afford ethyl (E)-4-[2-[2-[2-[2-[2-[bis(tert-butoxycarbonyl)amino]ethoxy]ethoxy]ethoxy]ethoxy]ethoxy]but-2-enoate (176 mg, 0.32 mmol, 28% yield) as a colorless oil. ¹H NMR (400 MHZ, CDCl₃): δ 7.00-6.91 (m, 1H), 6.12-6.04 (m, 1H), 4.24-4.17 (m, 4H), 3.83-3.77 (m, 2H), 3.68-3.64 (m, 12H), 3.63-3.61 (m, 6H), 1.51 (s, 18H), 1.30 (t, 3H, J=7.2 Hz). LC-MS: MS (ES⁺): RT=0.986 min, m/z=567.3 [M+H⁺+17].

### 38.2 Preparation of Compound 3

To a solution of ethyl (E)-4-[2-[2-[2-[2-[2-[bis(tert-butoxycarbonyl)amino]ethoxy]ethoxy]ethoxy]ethoxy]ethoxy]but-2-enoate (176 mg, 0.32 mmol, 1.0 equiv) in EtOH (1.5 mL) and H₂O (1.5 mL) was added LiOH·H₂O (40.3 mg, 0.96 mmol, 3.0 equiv). The mixture was stirred at 20° C. for 1 h. The pH of the reaction mixture was adjusted to 6˜7 by addition of 1 N aqueous HCl solution. Then the mixture was concentrated in vacuo to afford (E)-4-[2-[2-[2-[2-[2-[bis(tert-butoxycarbonyl)amino]ethoxy]ethoxy]ethoxy]ethoxy]ethoxy]but-2-enoic acid (220 mg, 0.31 mmol, 74% purity) as a yellow solid. ¹H NMR (400 MHZ, DMSO-d₆) δ 6.26-6.16 (m, 1H), 5.83-5.72 (m, 1H), 3.67-3.61 (m, 4H), 3.54-3.45 (m, 18H), 1.44 (s, 18H). LC-MS: MS (ES⁺): RT=1.128 min, m/z=539.1 [M+H⁺+17].

### 38.3 Preparation of Compound 4

A mixture of (E)-4-[2-[2-[2-[2-[2-[bis(tert-butoxycarbonyl)amino]ethoxy]ethoxy]ethoxy]ethoxy]ethoxy]but-2-enoic acid (100 mg, 0.14 mmol, 74% purity, 1.0 equiv), 3-(4-phenoxyphenyl)-1-[(3R)-3-piperidyl]pyrazolo[3,4-d]pyrimidin-4-amine (72.0 mg, 0.10 mmol, 69% purity, 0.7 equiv, TFA salt), EDCI (68.0 mg, 0.35 mmol, 2.5 equiv), HOBt (47.9 mg, 0.35 mmol, 2.5 equiv) and DIPEA (91.7 mg, 0.71 mmol, 5.0 equiv) in DMF (2 mL) was stirred at 20° C. for 15 h under N₂. The reaction mixture was quenched with water (5 mL) and extracted with dichloromethane and methanol (10/1, 10 mL*3). The combined organic phase was washed with brine (15 mL), dried over anhydrous Na₂SO₄, filtered and concentrated in vacuo. The residue was purified by prep-TLC on silica gel (dichloromethane/methanol=10/1) to afford tert-butyl N-[2-[2-[2-[2-[2-[(E)-4-[(3R)-3-[4-amino-3-(4-phenoxyphenyl) pyrazolo[3,4-d]pyrimidin-1-yl]-1-piperidyl]-4-oxo-but-2-enoxy]ethoxy]ethoxy ethoxy]ethoxy]ethyl]-N-tert-butoxycarbonyl-carbamate (105 mg, 0.12 mmol, 83% yield) as a yellow oil. LC-MS: MS (ES⁺): RT=0.978 min, m/z=890.4 [M+H⁺].

### 38.4 Preparation of Compound 5

A mixture of tert-butyl N-[2-[2-[2-[2-[2-[(E)-4-[(3R)-3-[4-amino-3-(4-phenoxyphenyl) pyrazolo[3,4-d]pyrimidin-1-yl]-1-piperidyl]-4-oxo-but-2-enoxy]ethoxy]ethoxy]ethoxy]ethoxy]ethyl]-N-tert-butoxycarbonyl-carbamate (105 mg,0.12 mmol, 1.0 equiv) in DCM (3 mL) and TFA (1 mL) was stirred at 20° C. for 2 h. The mixture was concentrated in vacuo. The residue was purified by prep-HPLC (TFA condition: column: 3_Phenomenex Luna C18 75*30 mm*3 μm; mobile phase: [water (0.1% TFA)-ACN]; B %: 24%-54%,7 min) to afford (E)-4-[2-[2-[2-[2-(2-aminoethoxy)ethoxy]ethoxy]ethoxy]ethoxy]-1-[(3R)-3-[4-amino-3-(4-phenoxyphenyl) pyrazolo[3,4-d]pyrimidin-1-yl]-1-piperidyl]but-2-en-1-one (55.0 mg, 0.07 mmol, 58% yield, TFA salt) as a colorless oil. LC-MS: MS (ES⁺): RT=0.721 min, m/z=690.2 [M+H⁺].

### 38.5 Preparation of Compound I-38

A mixture of (E)-4-[2-[2-[2-[2-(2-aminoethoxy)ethoxy]ethoxy]ethoxy]ethoxy]-1-[(3R)-3-[4-amino-3-(4-phenoxyphenyl) pyrazolo[3,4-d]pyrimidin-1-yl]-1-piperidyl]but-2-en-1-one (35.0 mg, 0.04 mmol, 1.0 equiv, TFA salt), 4-[(8-cyclopentyl-7-ethyl-5-methyl-6-oxo-7H-pteridin-2-yl)amino]-3-methoxy-benzoic acid (18.5 mg, 0.04 mmol, 1.0 equiv), EDCI (20.9 mg, 0.11 mmol, 2.5 equiv), HOBt (14.7 mg, 0.11 mmol, 2.5 equiv) and DIPEA (28.1 mg, 0.22 mmol, 5.0 equiv) in DMF (0.2 mL) was stirred at 20° C. for 15 h under N₂. The reaction mixture was quenched with water (5 mL) and extracted with dichloromethane and methanol (10/1, 10 mL*3). The combined organic phase was washed with brine (15 mL), dried over anhydrous Na₂SO₄, filtered and concentrated in vacuo. The residue was purified by prep-HPLC (FA condition: column: Phenomenex luna C18 150*25 mm*10 μm; mobile phase: [water (0.225% FA)-ACN]; B %: 23%-53%,10 min) to afford N-[2-[2-[2-[2-[2-[(E)-4-[(3R)-3-[4-amino-3-(4-phenoxyphenyl) pyrazolo[3,4-d]pyrimidin-1-yl]-1-piperidyl]-4-oxobut-2-enoxy]ethoxy]ethoxy]ethoxy]ethoxy]ethyl]-4-[(8-cyclopentyl-7-ethyl-5-methyl-6-oxo-7H-pteridin-2-yl)amino]-3-methoxy-benzamide (28.3 mg, 0.02 mmol, 56% yield, 94% purity) as a white solid. ¹H NMR (400 MHZ, DMSO-d₆) δ 8.45-8.38 (m, 2H), 8.25 (s, 1H), 7.84 (s, 1H), 7.66-7.64 (m, 2H), 7.60 (s, 1H), 7.51-7.47 (m, 2H), 7.46-7.40 (m, 2H), 7.21-7.16 (m, 1H), 7.16-7.10 (m, 4H), 6.70-6.62 (m, 1H), 6.59-6.44 (m, 1H), 4.80-4.60 (m, 1H), 4.39-4.29 (m, 1H), 4.25-4.22 (m, 1H), 4.17-4.08 (m, 2H), 4.01 (brs, 1H), 3.92 (s, 3H), 3.57-3.51 (m, 7H), 3.47-3.27 (m, 14H), 3.24 (s, 3H), 2.28-1.86 (m, 6H), 1.82-1.72 (m, 4H), 1.68-1.50 (m, 4H), 0.75 (t, 3H, J=7.6 Hz). LC-MS: MS (ES⁺): RT=2.098 min, m/z=1097.5 [M+H⁺]; LC-MS Method 25.

### Example 39—Synthesis of Compound I-39

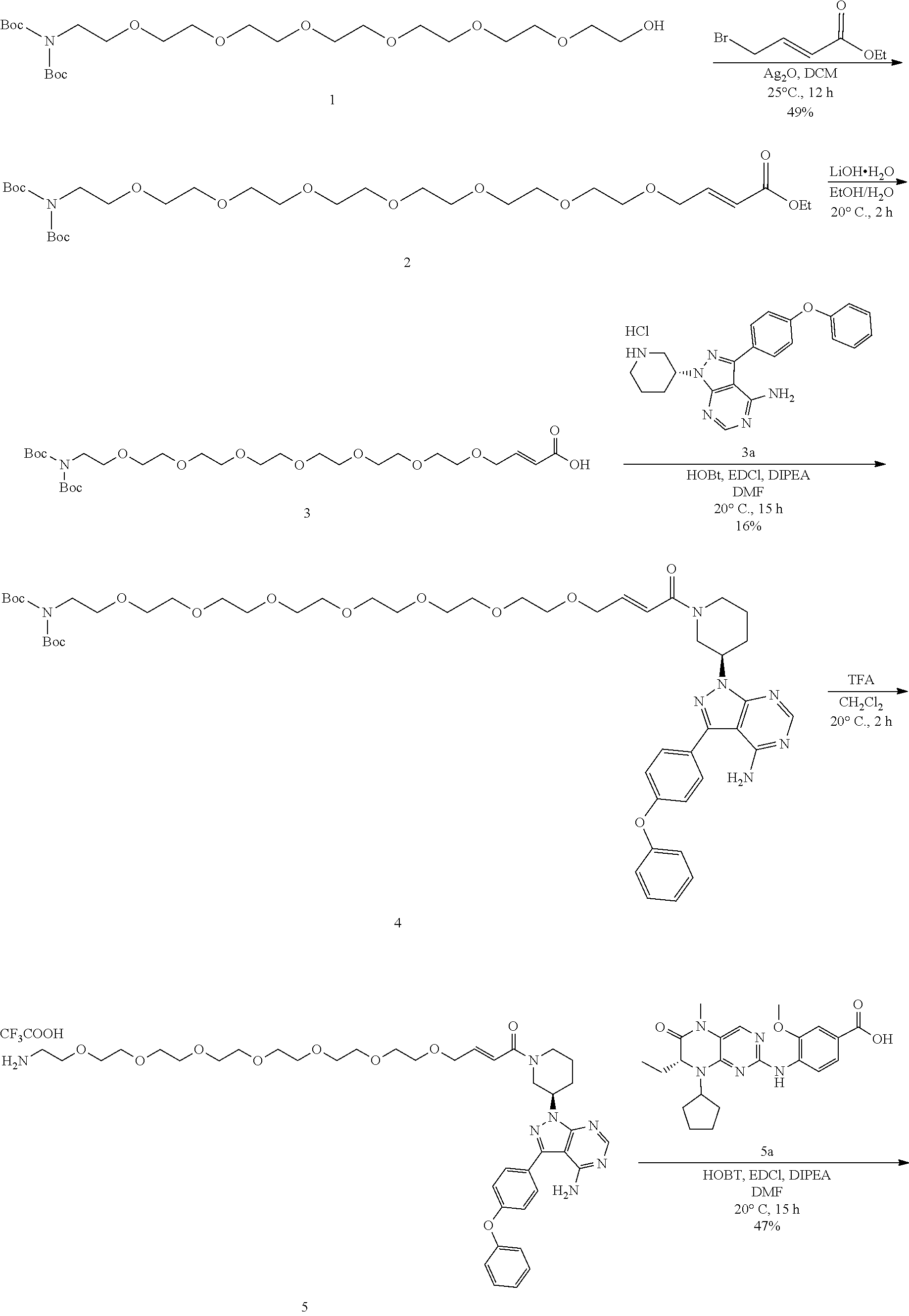

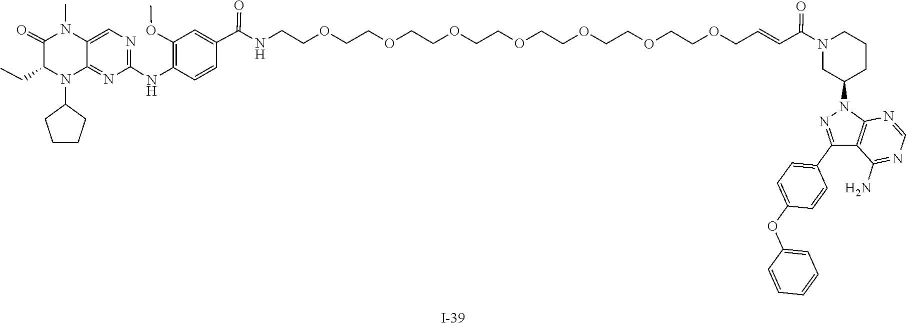

### 39.1 Preparation of Compound 2

To a solution of tert-butyl N-tert-butoxycarbonyl-N-[2-[2-[2-[2-[2-[2-(2-hydroxyethoxy)ethoxy]ethoxy]ethoxy]ethoxy]ethoxy]ethyl]carbamate (500 mg, 0.95 mmol, 1.0 equiv) in DCM (0.1 mL) were added Ag₂O (220 mg, 0.95 mmol, 1.0 equiv) and ethyl (E)-4-bromobut-2-enoate (734 mg, 3.80 mmol, 4.0 equiv) at 25° C. The mixture was stirred at 25° C. for 12 h. The reaction mixture was filtered and the filtrate concentrated in vacuo. The residue was purified by column chromatography on silica gel (petroleum ether/ethyl acetate=20/1 to 0/1) and then prep-TLC on silica gel (dichloromethane/methanol=20/1) to afford ethyl (E)-4-[2-[2-[2-[2-[2-[2-[2-[bis(tertbutoxycarbonyl)amino]ethoxy]ethoxy]-ethoxy]ethoxy]ethoxy]ethoxy]ethoxy]but-2-enoate (300 mg, 0.47 mmol, 49% yield) as a yellow oil. ¹H NMR (400 MHz, CDCl₃): δ 7.07-7.01 (m, 1H), 6.13-6.08 (m, 1H), 4.39-4.32 (m, 4H), 4.25-4.18 (m, 4H), 3.68-3.59 (m, 24H), 1.50 (s, 18H), 1.33-1.24 (m, 3H). LC-MS: MS (ES⁺): RT=0.990 min, m/z=655.2 [M+H⁺+17].

39.2 The preparation of compound 3

To a solution of ethyl (E)-4-[2-[2-[2-[2-[2-[2-[2-[bis(tertbutoxycarbonyl)amino]ethoxy]ethoxy]ethoxy]-ethoxy]ethoxy]ethoxy]ethoxy]but-2-enoate (362 mg, 0.57 mmol, 1.0 equiv) in EtOH (3 mL) and H₂O (3 mL) was added LiOH·H₂O (71.5 mg, 1.70 mmol, 3.0 equiv) at 20° C. The mixture was stirred at 20° C. for 2 h. The pH of the reaction mixture was adjusted to 6˜7 by addition of 1 N aqueous HCl solution. Then the mixture was concentrated in vacuo to afford (E)-4-[2-[2-[2-[2-[2-[2-[2-[bis(tert-butoxycarbonyl)amino]ethoxy]ethoxy]ethoxy]ethoxy]ethoxy]ethoxy]ethoxy]but-2-enoic acid (425 mg, crude) as a yellow solid. ¹H NMR (400 MHZ, DMSO-d₆) δ 6.29-6.21 (m, 1H), 5.80-5.69 (m, 1H), 3.96-3.94 (m, 2H), 3.64 (t, 2H, J=5.6 Hz), 3.52-3.45 (m, 26H), 1.44 (s, 18H). LC-MS: MS (ES⁺): RT=0.909 min, m/z=627.2 [M+H⁺+17].

39.3 The preparation of compound 4

A mixture of (E)-4-[2-[2-[2-[2-[2-[2-[2-[bis(tertbutoxycarbonyl)amino]ethoxy]ethoxy]ethoxy]ethoxy]-ethoxy]ethoxy]ethoxy]but-2-enoic acid (220 mg, 0.36 mmol, 1.0 equiv), 3-(4-phenoxyphenyl)-1-[(3R)-3-piperidyl]pyrazolo[3,4-d]pyrimidin-4-amine (153 mg, 0.36 mmol, 1.0 equiv, HCl salt), EDCI (83.0 mg, 0.43 mmol, 1.2 equiv), HOBt (58.5 mg, 0.43 mmol, 1.2 equiv) and DIPEA (233 mg, 1.80 mmol, 5.0 equiv) in DMF (5 mL) was stirred at 20° C. for 15 h under N₂. The reaction mixture was quenched with water (5 mL) and extracted with ethyl acetate (15 mL*3). The combined organic phase was washed with brine (10 mL), dried over anhydrous Na₂SO₄, filtered and concentrated in vacuo. The residue was purified by prep-HPLC (FA condition: column: Phenomenex luna C18 150*25 mm*10 μm; mobile phase: [water (0.2% FA)-ACN]; B %: 52%-82%,12 min) to afford tert-butyl N-[2-[2-[2-[2-[2-[2-[2-[(E)-4-[(3R)-3-[4-amino-3-(4-phenoxyphenyl) pyrazolo[3,4-d]pyrimidin-1-yl]-1-piperidyl]-4-oxo-but-2-enoxy]ethoxy]ethoxy]ethoxy]-ethoxy]ethoxy]ethoxy]ethyl]-N-tert-butoxycarbonyl-carbamate (55.0 mg,0.06 mmol, 16% yield) as a yellow oil. LC-MS: MS (ES⁺): RT=0.974 min, m/z=978.2 [M+H⁺].

39.4 The preparation of compound 5

A mixture of tert-butyl N-[2-[2-[2-[2-[2-[2-[2-[(E)-4-[(3R)-3-[4-amino-3-(4-phenoxyphenyl) pyrazolo[3,4-d]pyrimidin-1-yl]-1-piperidyl]-4-oxo-but-2-enoxy]ethoxy]ethoxy]ethoxy]ethoxy]ethoxy]ethoxy]ethyl]-N-tert-butoxycarbonyl-carbamate (55.0 mg, 0.06 mmol, 1.0 equiv) in DCM (3 mL) and TFA (1 mL) was stirred at 20° C. for 2 h. The mixture was concentrated in vacuo to afford (E)-4-[2-[2-[2-[2-[2-[2-(2-aminoethoxy)ethoxy]ethoxy]ethoxy]ethoxy]ethoxy]ethoxy]-1-[(3R)-3-[4-amino-3-(4-phenoxyphenyl) pyrazolo[3,4-d]pyrimidin-1-yl]-1-piperidyl]but-2-en-1-one (55.0 mg, crude, TFA salt) as a yellow oil. LC-MS: MS (ES⁺): RT=0.819 min, m/z=778.2 [M+H⁺].

39.5 The preparation of Compound I-39

A mixture of (E)-4-[2-[2-[2-[2-[2-[2-(2-aminoethoxy)ethoxy]ethoxy]ethoxy]ethoxy]ethoxy]ethoxy]-1-[(3R)-3-[4-amino-3-(4-phenoxyphenyl) pyrazolo[3,4-d]pyrimidin-1-yl]-1-piperidyl]but-2-en-1-one (55.0 mg, 0.06 mmol, 1.0 equiv, TFA salt), 4-[[(7R)-8-cyclopentyl-7-ethyl-5-methyl-6-oxo-7H-pteridin-2-yl]amino]-3-methoxy-benzoic acid (26.2 mg, 0.06 mmol, 1.0 equiv), HOBt (20.8 mg, 0.15 mmol, 2.5 equiv), EDCI (29.6 mg, 0.15 mmol, 2.5 equiv) and DIPEA (39.9 mg, 0.31 mmol, 5.0 equiv) in DMF (3 mL) was stirred at 20° C. for 15 h under N₂. The reaction mixture was quenched with water (5 mL) and extracted with ethyl acetate (10 mL*3). The combined organic phase was washed with brine (15 mL), dried over anhydrous Na₂SO₄, filtered and concentrated in vacuo. The residue was purified by prep-HPLC (neutral condition: column: Waters Xbridge 150*25 mm*5 μm; mobile phase: [water (10 mM NH₄HCO₃)-ACN]; B %: 41%-71%,9 min) to afford N-[2-[2-[2-[2-[2-[2-[2-[(E)-4-[(3R)-3-[4-amino-3-(4-phenoxyphenyl) pyrazolo[3,4-d]pyrimidin-1-yl]-1-piperidyl]-4-oxo-but-2-enoxy]ethoxy]ethoxy]ethoxy]ethoxy]ethoxy]ethoxy]ethyl]-4-[[(7R)-8-cyclopentyl-7-ethyl-5-methyl-6-oxo-7H-pteridin-2-yl]amino]-3-methoxy-benzamide (34.44 mg, 0.03 mmol, 47% yield, 99.7% purity) as an off-white solid. ¹H NMR (400 MHZ, DMSO-d₆) δ 8.44-8.36 (m, 2H), 8.25 (s, 1H), 7.84 (s, 1H), 7.69-7.62 (m, 2H), 7.59 (s, 1H), 7.52-7.46 (m, 2H), 7.45-7.41 (m, 2H), 7.21-7.10 (m, 5H), 6.71-6.64 (m, 1H), 6.61-6.45 (m, 1H), 4.83-4.49 (m, 1H), 4.39-4.29 (m, 1H), 4.25-4.22 (m, 1H), 4.17-3.99 (m, 3H), 3.93 (s, 3H), 3.56-3.37 (m, 26H), 3.29 (s, 2H), 3.24 (s, 3H), 2.15-1.87 (m, 7H), 1.82-1.72 (m, 5H), 1.68-1.53 (m, 5H), 0.76 (t, 3H, J=7.2 Hz). LC-MS: MS (ES⁺): RT=2.072 min, m/z=1184.8 [M+H⁺]; LC-MS Method 25.

### Example 40—Synthesis of Additional Compounds

Additional compounds set forth in the table below were prepared based on procedures described herein.

TABLE 2

| | | | | Retention | |
|---|---|---|---|---|---|
| | | MW | Observed | Time | LCMS |
| No. | Structure | Exact | Mass | (min) | Method |
| I-40 | 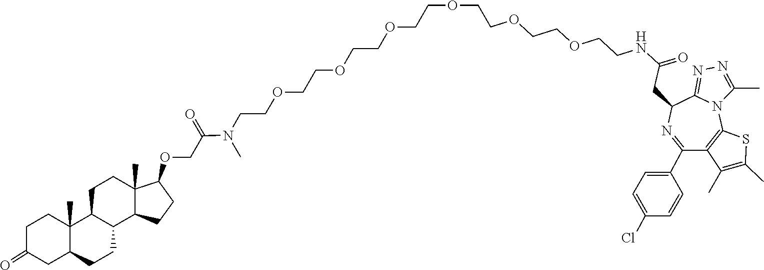 [SMILES: Cc1sc2c(c1C)C(c1ccc(Cl)cc1)=N[C@@H](CC(=O)NCCOCCOCCOCCOCCOCCOCCN(C)C(=O)CO[C@H]1CC[C@H]3[C@@H]4CC[C@H]5CC(=O)CC[C@]5(C)[C@H]4CC[C@]13C)c1nnc(C)n1-2] | 1050.5 | 526.3, 1051.4 | 220-2.791 | 25 |
| I-41 | 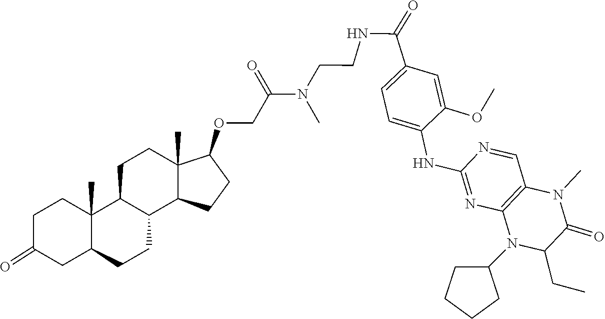 [SMILES: CCC1C(=O)N(C)c2cnc(Nc3ccc(C(=O)NCCN(C)C(=O)CO[C@H]4CC[C@H]5[C@@H]6CC[C@H]7CC(=O)CC[C@]7(C)[C@H]6CC[C@]45C)cc3OC)nc2N1C1CCCC1] | 811.5 | 406.8, 812.4 | 220-2.189 | 25 |
| I-42 | 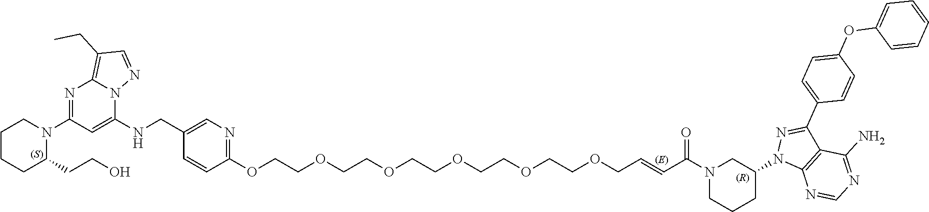 | 1068.6 | 535.4, 1069.6 | 220-2.221 | 25 |
| I-43 | 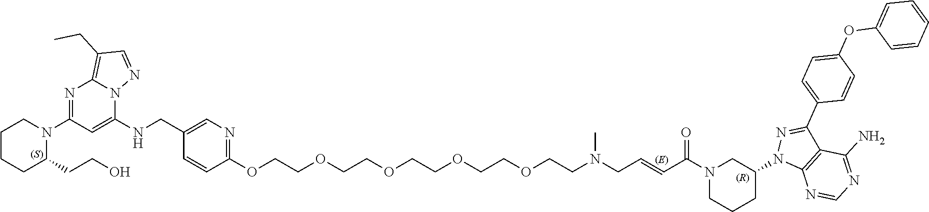 | 1081.6 | 541.8, 1082.5 | 220-1.939 | 25 |
| I-44 | 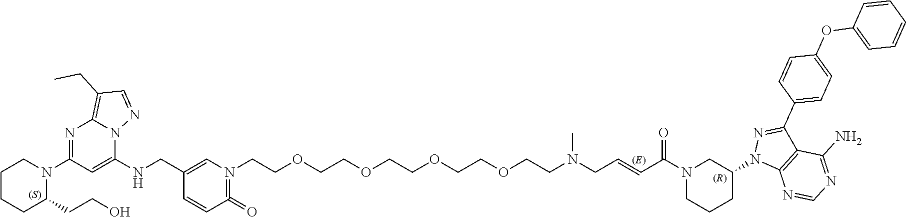 | 1081.6 | 361.6, 541.8 | 220-1.842 | 25 |
| I-45 | 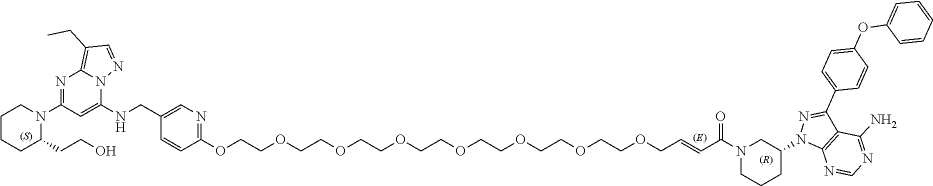 | 1156.6 | 386.7, 579.4 | 220-2.218 | 25 |
| I-46 | 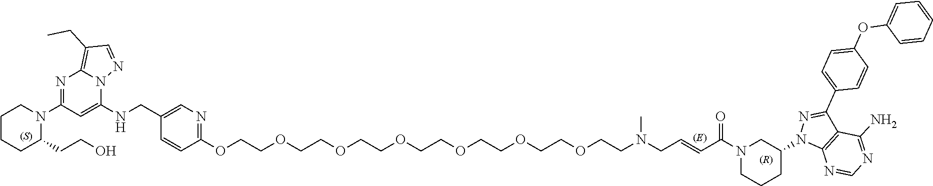 | 1169.6 | 586.0, 1170.7 | 220-1.906 | 25 |
| I-47 | 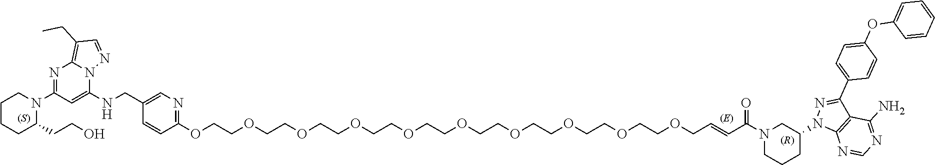 | 1244.7 | 416.0, 623.4 | 220-2.223 | 25 |
| I-48 | 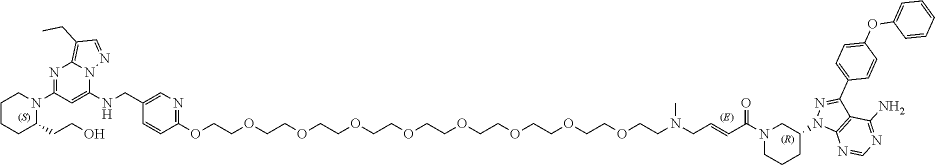 | 1257.7 | 630.0, 1258.7 | 220-1.884 | 25 |
| I-49 | 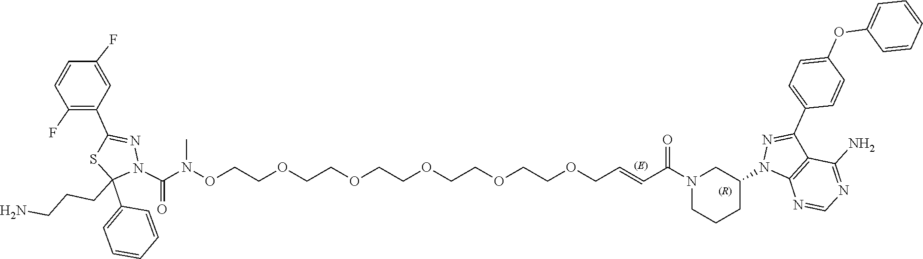 | 1087.5 | 540.3, 1079.3 | 220-2.283 | 25 |
| I-50 | 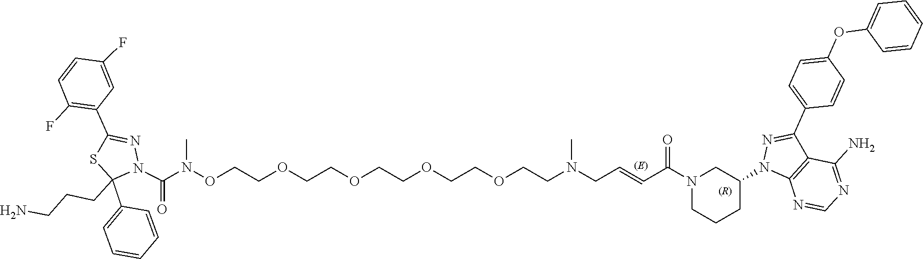 | 1091.5 | 546.8, 1092.4 | 220-1.942 | 25 |
| I-51 | 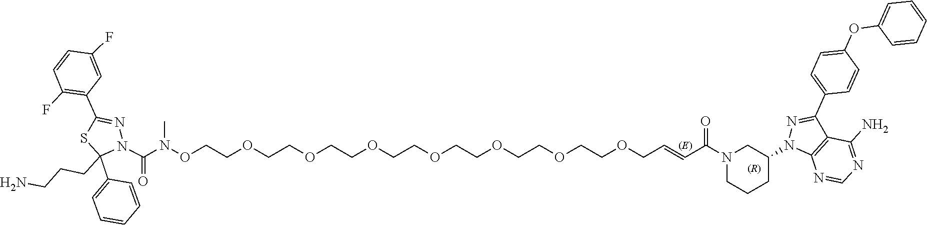 | 1166.5 | 584.4, 1167.6 | 220-2.298 | 25 |
| I-52 | 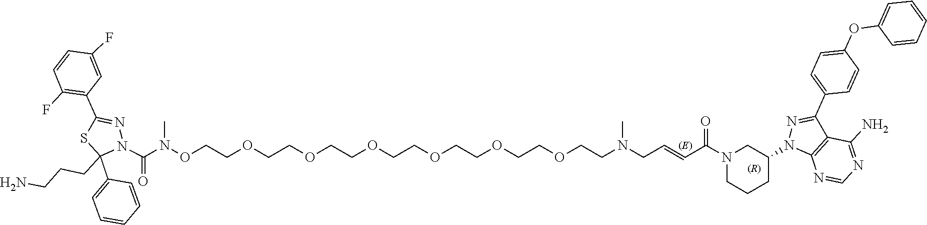 | 1179.5 | 590.8, 1180.5 | 220-1.958 | 25 |
| I-53 | 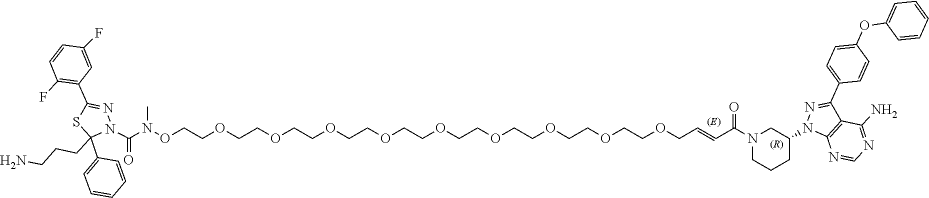 | 1254.6 | 628.3, 1255.4 | 220-2.314 | 25 |
| I-54 | 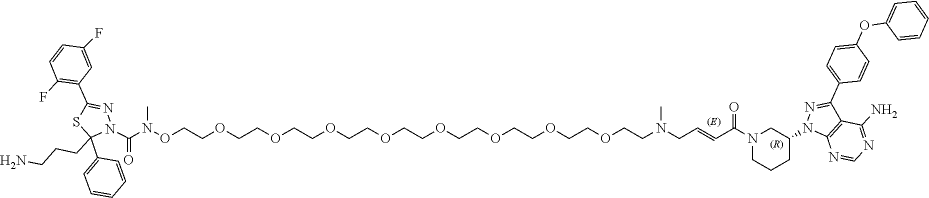 | 1267.6 | 634.8, 1268.4 | 220-1.957 | 25 |
| I-55 | 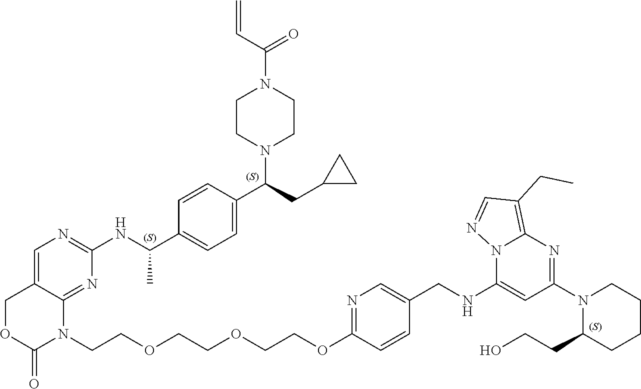 | 986.55 | 494.4, 987.5 | 220-2.187 | 10 |
| I-56 | 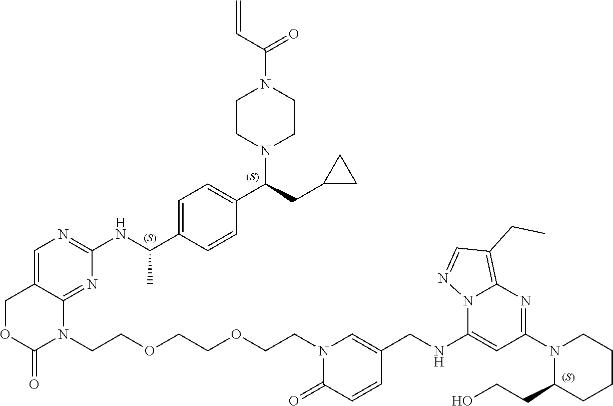 | 986.55 | 494.4, 987.5 | 220-2.113 | 10 |
| I-57 | 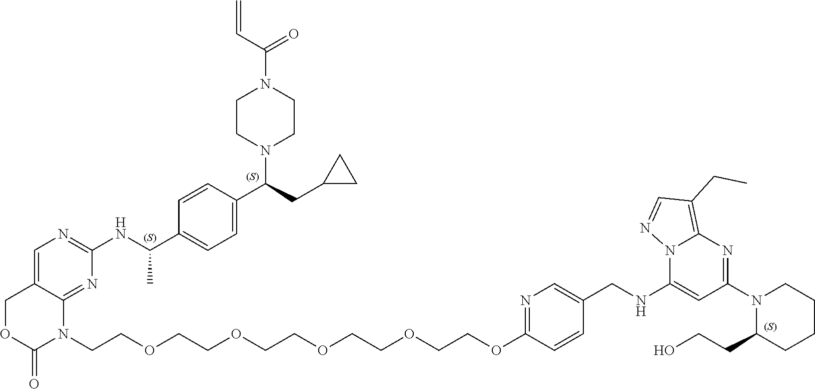 | 1074.6 | 359.2, 538.4 | 220-1.742 | 25 |
| I-58 | 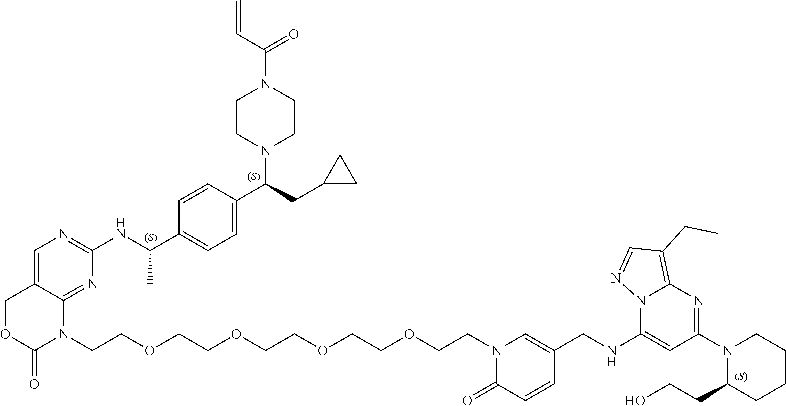 | 1074.6 | 359.3, 538.4 | 220-2.111 | 10 |
| I-59 | 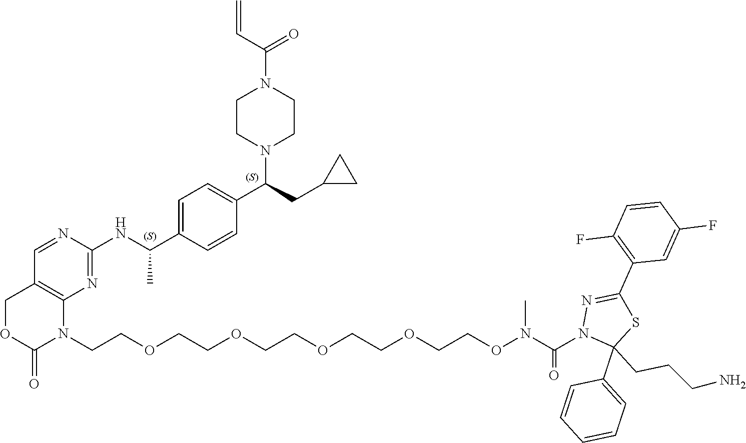 | 1084.5 | 543.3, 1085.5 | 220-1.811 | 25 |
| I-60 | 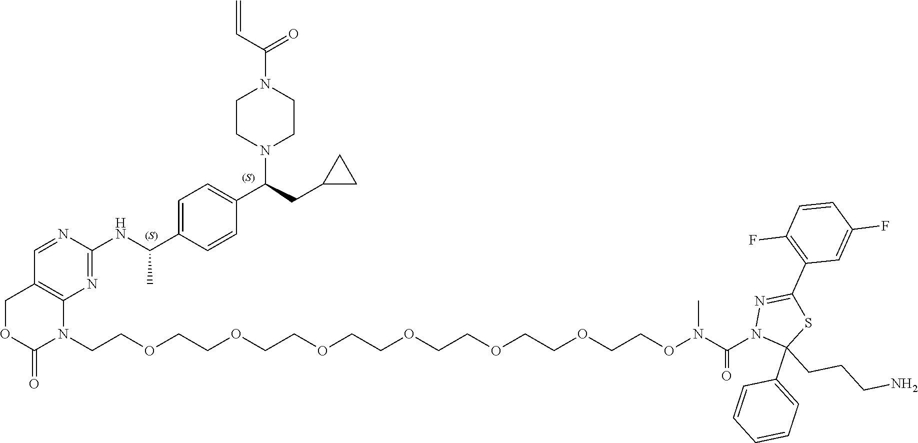 | 1172.6 | 587.4, 1173.6 | 220-1.854 | 25 |
| I-61 | 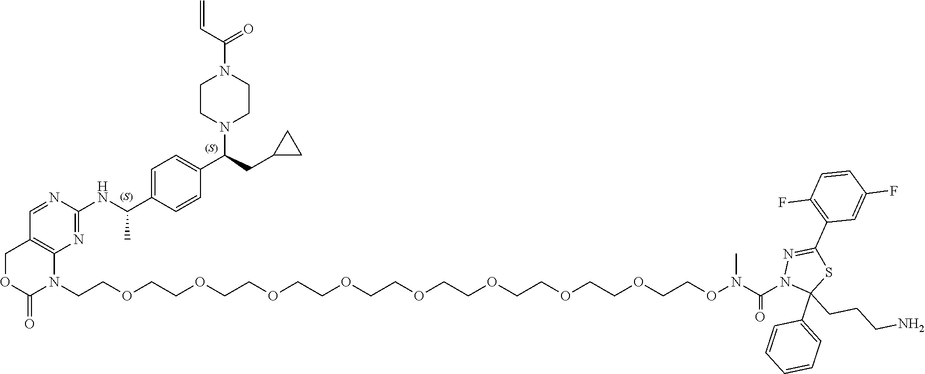 | 1260.6 | 631.4, 1261.6 | 220-1.863 | 25 |

### Example 41—Synthesis of Compound I-62

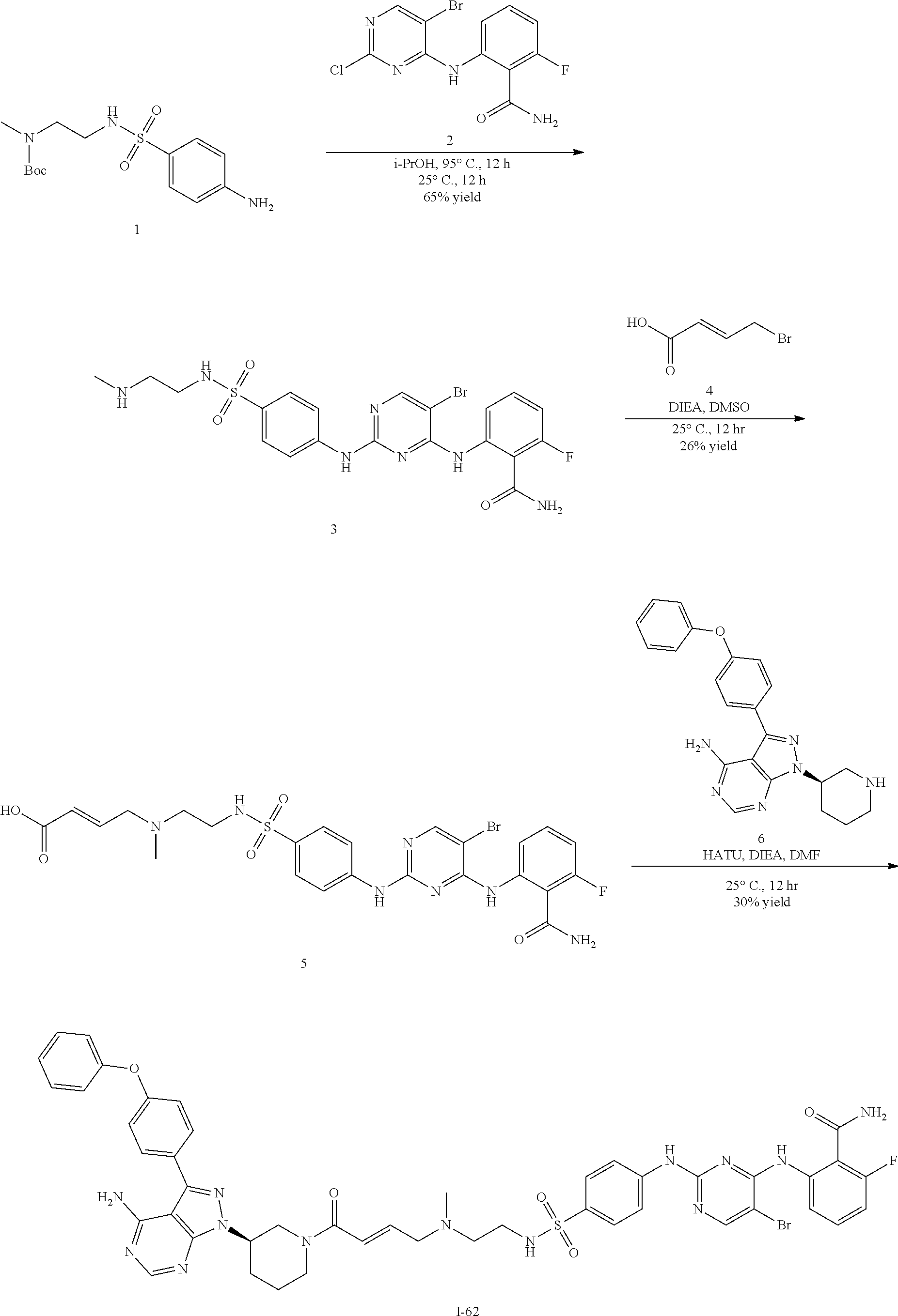

General Info: Compound 1 can be prepared as described in Journal of Medicinal Chemistry, 2019, 62, 266. Compound 2 can be prepared as described in WO 2020/206137. Compound 6 can be prepared as described in WO 2017/39425.

Step 1. A mixture of tert-butyl N-[2-[(4-aminophenyl)sulfonylamino]ethyl]-N-methyl-carbamate (1.4 g, 4.3 mmol, 1.0 equiv), 2-[(5-bromo-2-chloro-pyrimidin-4-yl)amino]-6-fluoro-benzamide (1.2 g, 3.4 mmol, 0.8 equiv), HCl (1 M, 4.3 mL, 1.0 equiv) in i-PrOH (20 mL) was degassed and purged with N₂ for 3 times, and then the mixture was stirred at 95° C. for 12 h under N₂ atmosphere. The mixture was cooled to 25° C., then the mixture was filtered. The solid was triturated with MeOH (30 mL) to give the 2-[[5-bromo-2-[4-[2-(methylamino) ethylsulfamoyl]anilino]pyrimidin-4-yl]amino]-6-fluoro-benzamide (1.5 g, 2.8 mmol, 65% yield) as a white solid. ¹H NMR: (400 MHZ, DMSO-d₆) δ=10.17 (d, J=12.0 Hz, 2H), 9.01 (d, J=3.6 Hz, 2H), 8.42 (s, 1H), 8.24-8.11 (m, 3H), 8.00-7.92 (m, 1H), 7.86 (d, J=8.8 Hz, 2H), 7.68 (d, J=8.8 Hz, 2H), 7.62-7.48 (m, 1H), 7.16-7.07 (m, 1H), 3.04-2.91 (m, 4H), 2.52 (s, 3H).

Step 2. To a solution of 2-[5-bromo-2-[4-[2-(methylamino) ethylsulfamoyl]anilino]pyrimidin-4-yl]amino]-6-fluoro-benzamide (300 mg, 557 μmol, 1.0 equiv) in DMSO (2 mL) was added (E)-4-bromobut-2-enoic acid (82 mg, 501 μmol, 0.9 equiv). Then the mixture was added DIEA (288 mg, 2.0 mmol, 388 μL, 4.0 equiv). The mixture was stirred under N₂ atmosphere at 25° C. for 12 h. The reaction mixture was concentrated under reduced pressure to give a residue. The crude product was purified by reversed-phase HPLC (column: Phenomenex C18 250*50 mm*10 μm; mobile phase: [water (NH₄HCO₃)-ACN]; B %: 8%-38%,8 min) to give the (E)-4-[2-[[4-[[5-bromo-4-(2-carbamoyl-3-fluoro-anilino) pyrimidin-2-yl]amino]phenyl]sulfonylamino]ethyl-methyl-amino]but-2-enoic acid (93 mg, 149 μmol, 26% yield) as a white solid. ¹H NMR: (400 MHZ, DMSO-d₆) δ=10.10 (s, 1H), 9.92 (s, 1H), 8.38 (s, 1H), 8.28-8.04 (m, 3H), 7.84 (d, J=8.8 Hz, 2H), 7.64 (d, J=8.8 Hz, 2H), 7.56-7.45 (m, 1H), 7.40-7.28 (m, 1H), 7.17-6.98 (m, 1H), 6.82-6.57 (m, 1H), 5.87 (d, J=15.6 Hz, 1H), 3.09-3.01 (m, 2H), 2.86-2.75 (m, 2H), 2.38-2.27 (m, 2H), 2.07 (s, 3H).

Step 3. To a solution of (E)-4-[2-[[4-[[5-bromo-4-(2-carbamoyl-3-fluoro-anilino) pyrimidin-2-yl]amino]phenyl]sulfonylamino]ethyl-methyl-amino]but-2-enoic acid (40 mg, 64 μmol, 1.0 equiv) in DMF (0.5 mL) was added 3-(4-phenoxyphenyl)-1-[(3R)-3-piperidyl]pyrazolo[3,4-d]pyrimidin-4-amine (24 mg, 64 μmol, 1.0 equiv). Then the mixture was added DIEA (24 mg, 192 μmol, 33 L, 3.0 equiv) and HATU (24 mg, 64 μmol, 1.0 equiv). The mixture was stirred at 25° C. for 12 h. The reaction mixture was concentrated under reduced pressure to give a residue. The crude product was purified by reversed-phase HPLC (column: Waters xbridge 150*25 mm 10 μm; mobile phase: [water (NH₄HCO₃)-ACN]; B %: 38%-68%,11 min) to give the 2-[[2-[4-[2-[[(E)-4-[(3R)-3-[4-amino-3-(4-phenoxyphenyl) pyrazolo[3,4-d]pyrimidin-1-yl]-1-piperidyl]-4-oxo-but-2-enyl]-methyl-amino]ethylsulfamoyl]anilino]-5-bromo-pyrimidin-4-yl]amino]-6-fluoro-benzamide (20 mg, 19 μmol, 30% yield, 97% purity) as a white solid. ¹H NMR: (400 MHZ, DMSO-d₆) δ=10.12 (s, 1H), 10.02-9.84 (m, 1H), 8.38 (s, 1H), 8.29-8.21 (m, 2H), 8.14 (d, J=19.6 Hz, 2H), 7.89-7.79 (m, 2H), 7.69-7.58 (m, 4H), 7.55-7.47 (m, 1H), 7.47-7.31 (m, 3H), 7.24-7.07 (m, 6H), 6.62 (s, 2H), 4.86-4.64 (m, 1H), 4.58-4.00 (m, 2H), 3.23-2.92 (m, 5H), 2.85-2.74 (m, 2H), 2.30-2.21 (m, 2H), 2.19-1.82 (m, 6H), 1.63-1.49 (m, 1H). LC-MS: MS (ES⁺): RT=2.482 min, m/z=496.6 [M+H⁺]. LCMS Method 05.

### Example 42—Synthesis of Compound I-63 HLDA-004351

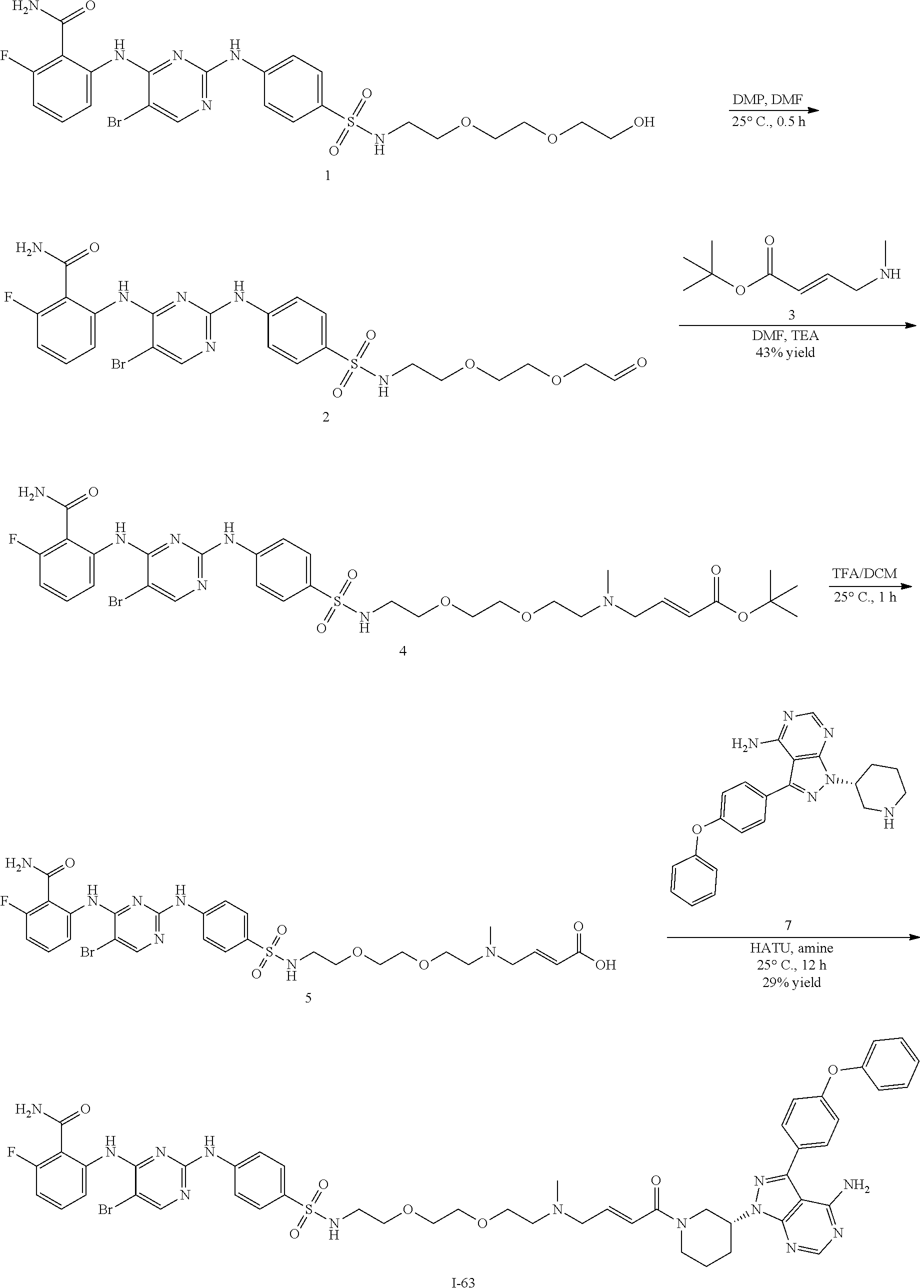

Step 1. To a stirred solution of 2-((5-bromo-2-((4-(N-(2-(2-(2-hydroxyethoxy) ethoxy) ethyl) sulfamoyl)phenyl)amino)pyrimidin-4-yl)amino)-6-fluorobenzamide (90 mg, 144 μmol, 1.0 equiv) in DMF (2 mL) was added DMP (183 mg, 431 μmol, 134 μL, 3.0 equiv). The reaction mixture was stirred at 25° C. for 0.5 h under N₂. The reaction mixture was used for next step directly.

Step 2. To a stirred solution of 2-((5-bromo-2-((4-(N-(2-(2-(2-oxoethoxy) ethoxy) ethyl) sulfamoyl)phenyl)amino)pyrimidin-4-yl)amino)-6-fluorobenzamide (87 mg, 142 μmol, 1.0 equiv) and tert-butyl(E)-4-(methylamino)but-2-enoate (73 mg, 427 μmol, 3.0 equiv) in DMF (5 mL) was added TEA (216 mg, 2.1 mmol, 297 μL, 15.0 equiv) and sodium; triacetoxyboranuide (452 mg, 2.1 mmol, 15.0 equiv). The reaction mixture was stirred at 25° C. for 12 h under N₂. The crude product was purified by reversed-phase HPLC (column: Waters xbridge 150*25 mm 10 μm; mobile phase: [water (NH₄HCO₃)-ACN]; B %: 40%-70%, 11 min). Tert-butyl(E)-4-((2-(2-(2-((4-((5-bromo-4-((2-carbamoyl3fluorophenyl)amino)pyrimidin2yl) amino)phenyl) sulfonamido)ethoxy)ethoxy) ethyl)(methyl)amino)but-2-enoate (47 mg, 61 μmol, 43% yield) was obtained as a white solid.

Step 3. To a stirred solution of Tert-butyl(E)-4-((2-(2-(2-((4-((5-bromo-4-((2-carbamoyl3fluorophenyl)amino)pyrimidin2yl)amino)phenyl) sulfonamido)ethoxy)ethoxy) ethyl)(methyl)amino)but-2-enoate (47.0 mg, 61.3 μmol, 1.0 equiv) in DCM (2 mL) was added TFA (500 mg, 4.39 mmol, 325 μL, 71.5 equiv). The reaction mixture was stirred at 25° C. for 1 hr under N₂. Concentrated under reduced pressure to give (E)-4-((2-(2-(2-((4-((5-bromo-4-((2-carbamoyl-3-fluorophenyl)amino)pyrimidin-2-yl)amino)phenyl) sulfonamido)ethoxy) ethoxy) ethyl)(methyl)amino)but-2-enoic acid (50 mg, 60 μmol, TFA) was obtained as a yellow oil and was used for next step directly.

Step 4. To a stirred solution of (E)-4-((2-(2-(2-((4-((5-bromo-4-((2-carbamoyl-3-fluorophenyl)amino)pyrimidin-2-yl)amino)phenyl) sulfonamido)ethoxy)ethoxy) ethyl)(methyl)amino)but-2-enoic acid (43 mg, 85.9 μmol, 100% purity, 1.4 equiv, TFA) and (R)-3-(4-phenoxyphenyl)-1-(piperidin-3-yl)-1H-pyrazolo[3,4-d]pyrimidin-4-amine (50 mg, 60 μmol, 100% purity, 1.0 equiv, TFA) in DMF (2 mL) was added DIEA (47 mg, 364 μmol, 63 μL, 6.0 equiv), HATU (69 mg, 182 μmol, 3.0 equiv). The reaction mixture was stirred at 25° C. for 12 h under N₂. The mixture was filtered and concentrated under reduced pressure to give a residue. The crude product was purified by reversed-phase HPLC (column: Phenomenex C18 150*25 mm*10 μm; mobile phase: [water (NH₄HCO₃)-ACN]; B %: 42%-72%,8 min) to give the (R,E)-2-((2-((4-(N-(2-(2-(2-((4-(3-(4-amino-3-(4-phenoxyphenyl)-1H-pyrazolo[3,4-d]pyrimidin-1-yl) piperidin-1-yl)-4-oxobut-2-en-1-yl)(methyl)amino)ethoxy)ethoxy) ethyl) sulfamoyl)phenyl)amino)-5-bromopyrimidin-4-yl)amino)-6-fluorobenzamide (20 mg, 18 μmol, 29% yield, 96.0% purity) as a white solid. 1H NMR (400 MHZ, DMSO-d₆) δ=10.09 (s, 1H), 9.91 (s, 1H), 8.35 (s, 1H), 8.18-8.24 (m, 2H), 8.14 (s, 1H), 8.09 (s, 1H), 7.81 (d, J=8.88 Hz, 2H), 7.58-7.66 (m, 4H), 7.56-7.69 (m, 1H), 7.44-7.52 (m, 1H), 7.37-7.44 (m, 2H), 7.02-7.22 (m, 7H), 6.57-6.64 (m, 1H), 6.46 (d, J=7.13 Hz, 1H), 4.58-4.77 (m, 1H), 4.05-4.17 (m, 1H), 3.41 (s, 5H), 3.28 (s, 8H), 3.06-3.21 (m, 3H), 2.93-3.00 (m, 1H), 2.77-2.91 (m, 3H), 2.14 (s, 1H), 2.05 (s, 1H), 1.85-1.93 (m, 1H), 1.48-1.60 (m, 1H). LC-MS: MS (ES⁺): RT=2.559 min, m/z=1078.2 [M+H⁺]; LCMS method 05.

### Example 43—the Synthetic Route for I-64

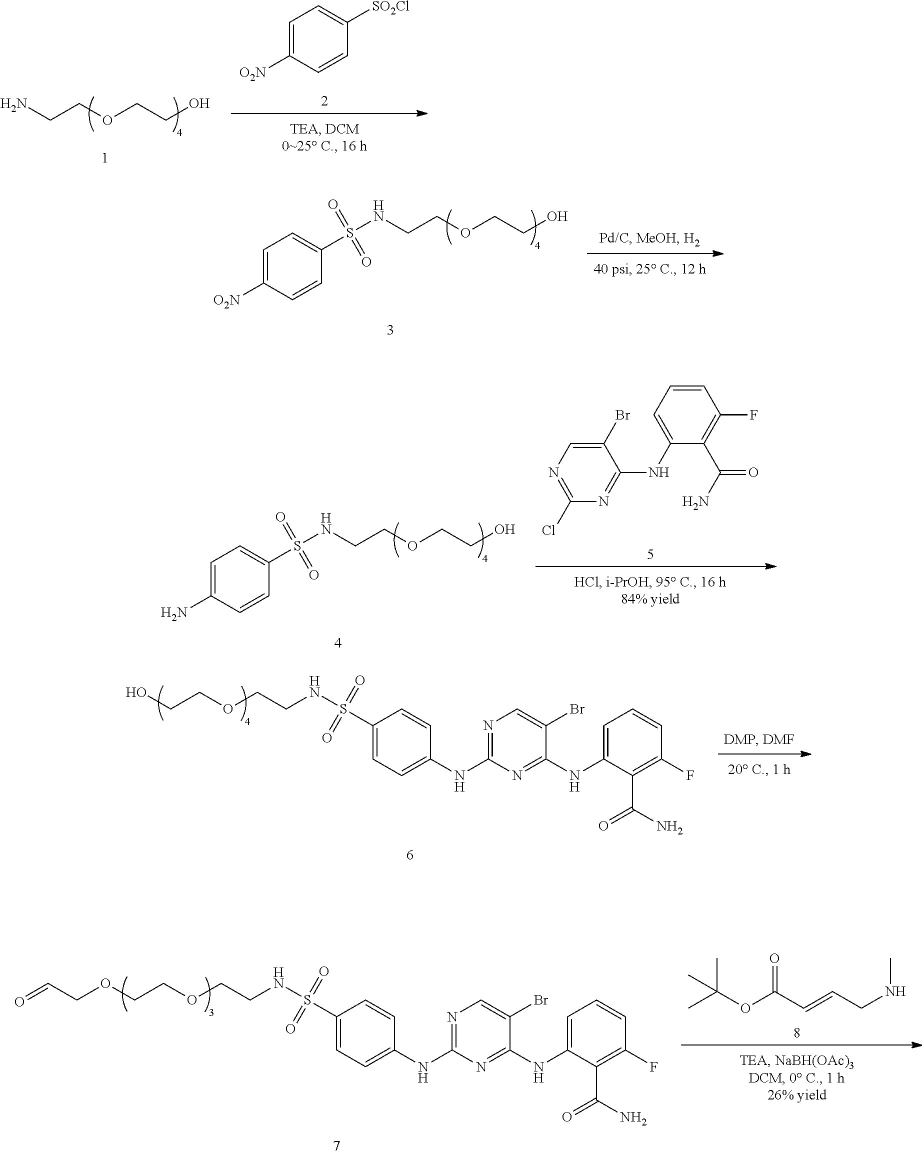

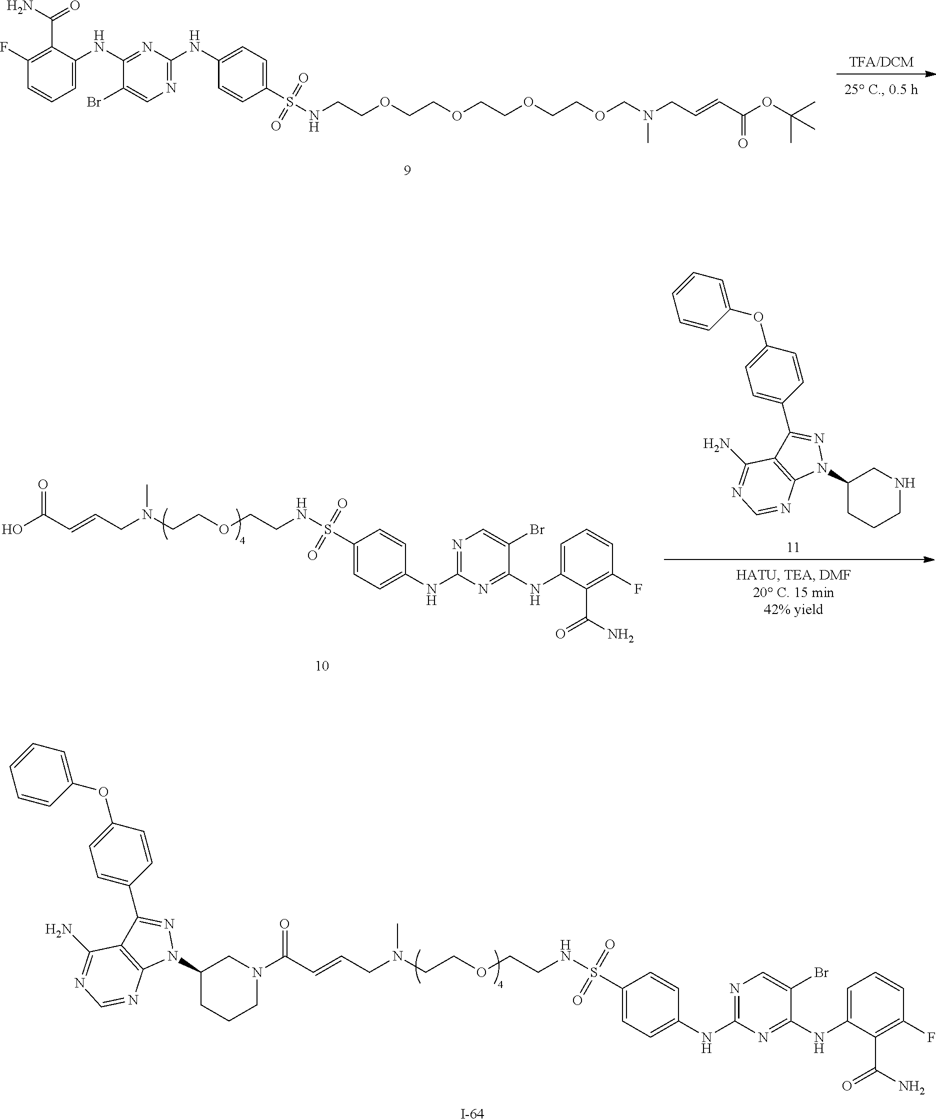

General Information: Synthetic route for compound 5 is described in Angewandte Chemie—International Edition, 2020, 59, 13865. Synthetic route for compound 11 is described in WO2017/39425, 2017, A¹.

Step 1. To a solution of 2-[2-[2-[2-(2-aminoethoxy)ethoxy]ethoxy]ethoxy]ethanol (1.3 g, 5.4 mmol, 1.0 equiv), TEA (1.6 g, 16.2 mmol, 2.3 mL, 3.0 equiv) in DCM (10 mL) was added a solution of 4-nitrobenzenesulfonyl chloride (1.2 g, 5.4 mmol, 1.0 equiv) in DCM (15 mL) at 0° C. under N₂. The mixture was stirred at 25° C. for 16 h, The reaction mixture was quenched by addition 0.1M HCl (15 mL) and then diluted with 10% NaOH until the pH=7˜8 and extracted with DCM (15 mL×3). Dried over Na₂SO₄ filtered and concentrated under reduced pressure to afford N-[2-[2-[2-[2-(2-hydroxyethoxy)ethoxy]ethoxy]ethoxy]ethyl]-4-nitro-benzenesulfonamide (1.52 g, crude) as yellow solid. ¹H NMR: (400 MHZ, DMSO-d₆) δ 8.24-8.18 (m, 2H), 7.85-7.83 (m, 2H), 3.63-3.42 (m, 14H), 3.14-3.05 (m, 6H).

Step 2. To a solution of N-[2-[2-[2-[2-(2-hydroxyethoxy)ethoxy]ethoxy]ethoxy]ethyl]-4-nitro-benzenesulfonamide (1.5 g, 3.61 mmol, 1.0 equiv) in MeOH (10 mL) was added Pd/C (0.5 g, 10% purity) under H₂ atmosphere. The mixture was stirred at 25 C for 12 h. The reaction mixture was filtered and concentrated under reduced pressure to give a residue. The residue was purified by column chromatography (SiO₂, Dichloromethane/Methanol=20/1 to 10/1) to afford 4-amino-N-[2-[2-[2-[2-(2-hydroxyethoxy)ethoxy]ethoxy]ethoxy]ethyl]benzenesulfonamide (1.3 g, crude) as yellow solid.

Step 3. To a solution of 4-amino-N-[2-[2-[2-[2-(2-hydroxyethoxy)ethoxy]ethoxy]ethoxy]ethyl]benzenesulfonamide (0.5 g, 1.3 mmol, 1.0 equiv) in i-PrOH (5 mL) was added 2-[(5-bromo-2-chloro-pyrimidin-4-yl)amino]-6-fluoro-benzamide (440 mg, 1.27 mmol, 1.0 equiv) and HCl (12 M, 106 μL, 1.0 equiv). The mixture was stirred at 95° C. for 16 h. The reaction mixture concentrated under reduced pressure to give a residue. The residue was purified by prep-HPLC (column: YMC Triart C18 250*50 mm*7 μm; mobile phase: [water (NH₄HCO₃)-ACN]; B %: 35%-65%, 8 min] to afford 2-[[5-bromo-2-[4-[2-[2-[2-[2-(2-hydroxyethoxy)ethoxy]ethoxy]ethoxy]ethylsulfamoyl]anilino]pyrimidin-4-yl]amino]-6-fluoro-benzamide (750 mg, 1.1 mmol, 84% yield) as yellow solid. ¹H NMR: (400 MHZ, DMSO-d₆) δ 8.41 (s, 1H), 8.22-8.05 (m, 4H), 7.82 (d, J=8.4 Hz, 2H), 7.65 (d, J=8.4 Hz, 2H), 7.11 (t, J=9.0 Hz, 1H), 3.55-3.41 (m, 18H), 3.17 (s, 1H), 2.87 (s, 2H).

Step 4. A mixture of 2-[[5-bromo-2-[4-[2-[2-[2-[2-(2-hydroxyethoxy)ethoxy]ethoxy]ethoxy]ethylsulfamoyl]anilino]pyrimidin-4-yl]amino]-6-fluoro-benzamide (400 mg, 570 μmol, 1.0 equiv), DMP (725 mg, 1.71 mmol, 529 μL, 3.0 equiv), TEA (58 mg, 570 μmol, 79 μL, 1.0 equiv) in DMF (4 mL), and then the mixture was stirred at 20° C. for 1 h. The mixture was filtration. The mixture was use to next step directly.

Step 5. A mixture of 2-[[5-bromo-2-[4-[2-[2-[2-[2-(2-oxoethoxy)ethoxy]ethoxy]ethoxy]ethylsulfamoyl]anilino]pyrimidin-4-yl]amino]-6-fluoro-benzamide (0.2 g, 286 μmol, 1.0 equiv) tert-butyl(E)-4-(methylamino)but-2-enoate (147 mg, 858 μmol, 3.0 equiv) in DCM (0.5 mL) added NaBH (OAc)3 (909 mg, 4.3 mmol, 15.0 equiv) and TEA (434 mg, 4.3 mmol, 597 μL, 15.0 equiv). The mixture was stirred at 0° C. for 1 h. The reaction mixture was quenched by addition sat. NaHCO₃ (50 mL), and then extracted with DCM (10 mL×3), dried over Na₂SO₄, filtered and concentrated under reduced pressure to give a residue. The residue was purified by prep-HPLC (column: Waters xbridge 150*25 mm 10 μm; mobile phase: [water (NH₄HCO₃)-ACN]; B %: 38%-68%,8 min) to afford tert-butyl(E)-4-[2-[2-[2-[2-[2[[4[[5bromo4 (2carbamoyl3fluoroanilino) pyrimidin2yl]amino]phenyl]sulfonylamino]ethoxy]ethoxy]ethoxy]ethoxy]ethyl-methyl-amino]but-2-enoate (64 mg, 75 μmol, 26% yield) as white solid. ¹H NMR: (400 MHz, DMSO-d₆) δ=10.13 (s, 1H), 9.93 (s, 1H), 8.39 (s, 1H), 8.24 (d, J=8.4 Hz, 1H), 8.15 (d, 20 Hz, 2H), 7.84 (d, J=8.8 Hz, 2H), 7.64 (d, J=8.8 Hz, 2H), 7.56-7.45 (m, 2H), 7.09 (t, J=9.2 Hz, 1H), 6.72-6.80 (m, 1H), 5.88 (d, J=15.6 Hz, 1H), 3.51-3.45 (m, 10H), 3.44 (d, J=4.0 Hz, 4H), 3.40-3.36 (m, 4H), 2.87 (t, J=5.8 Hz, 2H), 2.15 (s, 3H), 1.42 (s, 9H).

Step 6. A mixture of tert-butyl(E)-4-[2-[2-[2-[2-[2-[4-[5-bromo-4-(2-carbamoyl-3-fluoro-anilino) pyrimidin-2-yl]amino]phenyl]sulfonylamino]ethoxy]ethoxy]ethoxy]ethoxy]ethyl-methyl-amino]but-2-enoate (40 mg, 47 μmol, 1.0 equiv), TFA (1 mL) in DCM (3 mL) was degassed and purged with N for 3 times, and then the mixture was stirred at 25 C for 0.5 h under N₂ atmosphere. The reaction mixture was concentrated under reduced pressure to give a residue. The mixture was use to next step directly.

Step 7. To a solution of (E)-4-[2-[2-[2-[2-[2-[[4-[[5-bromo-4-(2-carbamoyl-3-fluoro-anilino) pyrimidin-2-yl]amino]phenyl]sulfonylamino]ethoxy]ethoxy]ethoxy]ethoxy]ethyl-methyl-amino]but-2-enoic acid (50 mg, 63 μmol, 1.0 equiv) and (1S)-3-(4-phenoxyphenyl)-1-(3-piperidyl) pyrazolo[3,4-d]pyrimidin-4-amine (24 mg, 48 μmol, 1.2 equiv, TFA) in DMF (2 mL) was added DIEA (40 mg, 313 μmol, 54 μL, 5.0 equiv) and HATU (48 mg, 125 μmol, 2.0 equiv). The mixture was stirred at 20° C. for 15 min. The reaction mixture was concentrated under reduced pressure to give a residue. The residue was purified by prep-HPLC (column: Waters xbridge 150*25 mm 10 μm; mobile phase: [water (NH₄HCO₃)-ACN]; B %: 36%-66%,8 min) to afford 2-[[2-[4-[2-[2-[2-[2-[2-[[(E)-4-[(3R)-3-[4-amino-3-(4-phenoxyphenyl) pyrazolo[3,4-d]pyrimidin-1-yl]-1-piperidyl]-4-oxo-but-2-enyl]-methyl-amino]ethoxy]ethoxy]ethoxy]ethoxy]ethylsulfamoyl]anilino]-5-bromo-pyrimidin-4-yl]amino]-6-fluoro-benzamide (35 mg, 26 μmol, 42% yield,88% purity) as white solid. ¹H NMR: (400 MHz, DMSO-d₆) δ=10.12 (s, 1H), 9.93 (s, 1H), 8.38 (s, 1H), 8.26-8.21 (m, 2H), 8.16 (s, 1H), 8.11 (s, 1H), 7.83 (d, J=8.8 Hz, 2H), 7.66-7.61 (m, 4H), 7.54-7.47 (m, 2H), 7.43 (t, J=8.0 Hz, 2H), 7.19-7.09 (m, 6H), 6.66-6.56 (m, 1H), 4.72 (s, 1H), 4.20-4.11 (m, 1H), 3.78-3.66 (m, 1H), 3.51-3.36 (m, 19H), 3.30 (s, 3H), 3.24-3.15 (m, 2H), 3.06-2.98 (m, 1H), 2.86 (d, J=5.6 Hz, 2H), 2.68-2.65 (m, 2H), 2.34-2.31 (m, 2H), 2.28-2.21 (m, 1H), 2.16-2.08 (m, 2H). LC-MS: MS (ES⁺): RT=18.474 min, m/z=584.7 [1/2M+H+]; LCMS method 05.

### Example 44—the Synthetic Route for I-65

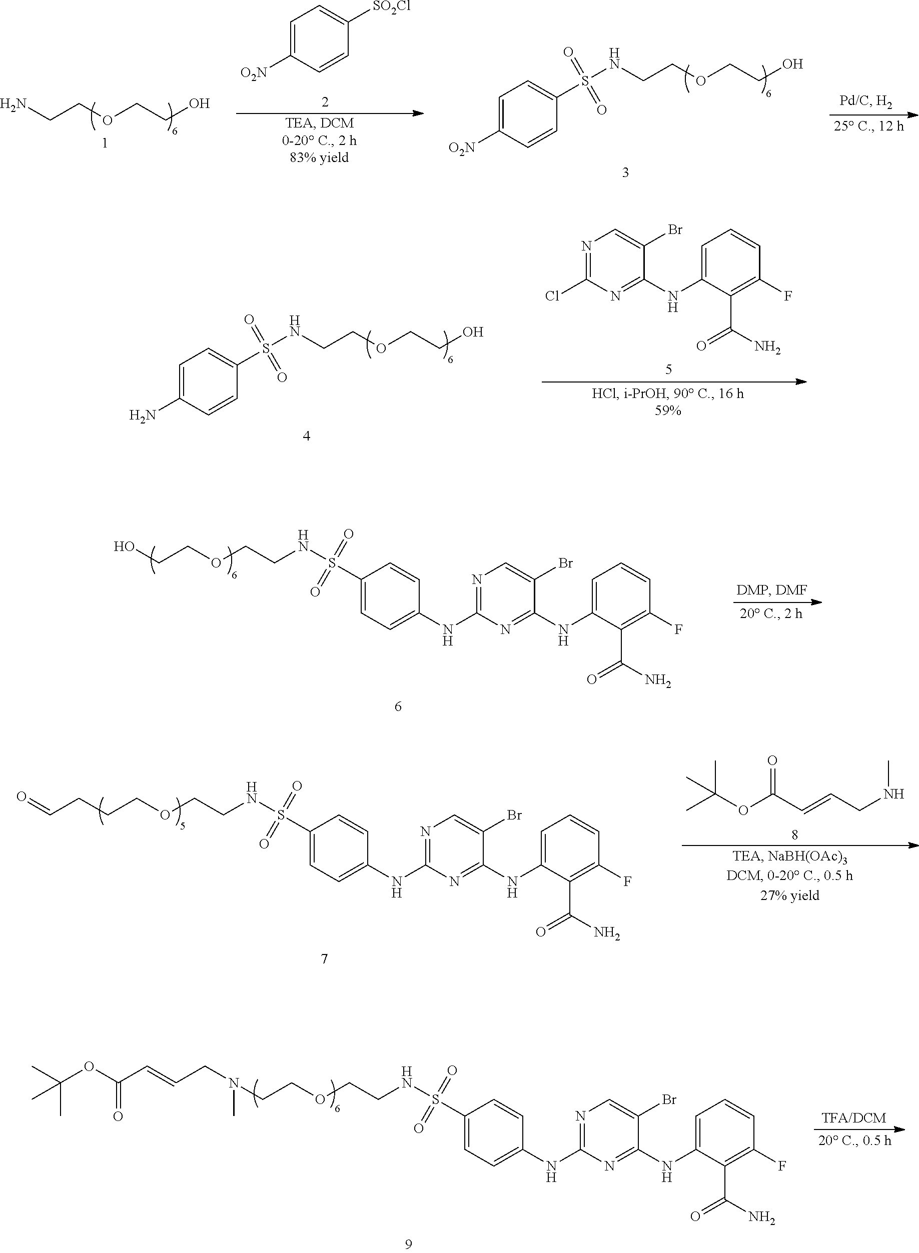

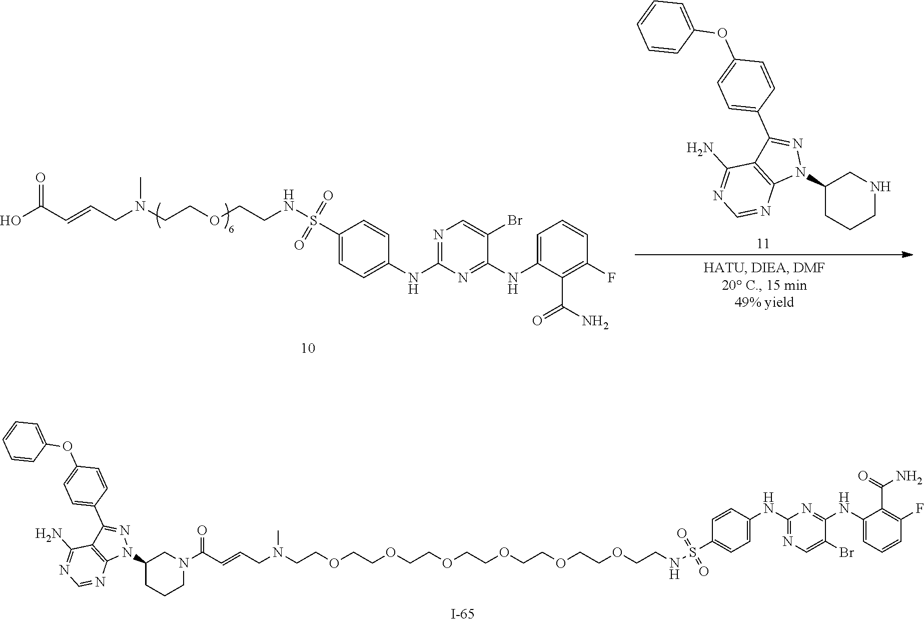

General Information: Synthetic route for compound 5 is described in Angewandte Chemie—International Edition, 2020, 59, 13865. Synthetic route for compound 11 is described in WO2017/39425. 2017, A1.

Step 1. To a solution of 2-[2-[2-[2-[2-[2-(2-aminoethoxy)ethoxy]ethoxy]ethoxy]ethoxy]ethoxy]ethanol (1.0 g, 3.1 mmol, 1.0 equiv) and TEA (1.2 g, 12.3 mmol, 1.7 mL, 4.0 equiv) in DCM (10 mL) was added a solution of 4-nitrobenzenesulfonyl chloride (681 mg, 3.1 mmol, 1.0 equiv) in DCM (2 mL) at 0° C. under N₂. The mixture was stirred at 20° C. for 2 h. The reaction mixture was quenched by addition HCl (0.5 mol, 50 mL) at 20° C., and adjust PH to 7˜8 by sat. NaHCO₃. extracted with DCM (20 mL×3). The combined organic layers were dried over Na₂SO₄, filtered and concentrated under reduced pressure to give a residue. The residue was purified by silica chromatography (PE: EtOAc=1: 0˜10:1) to afford N-[2-[2-[2-[2-[2-[2-(2-hydroxyethoxy)ethoxy]ethoxy]ethoxy]ethoxy]ethoxy]ethyl]-4-nitro-benzenesulfonamide (1.3 g, 2.6 mmol, 83% yield) as a yellow oil. ¹H NMR: (400 MHZ, CD₃OD) δ 8.41 (d, J=8.8 Hz, 2H), 8.11 (d, J=8.8 Hz, 2H), 5.49 (s, 1H), 3.63 (s, 19H), 3.58-3.53 (m, 4H), 3.50-3.44 (m, 4H), 3.14 (t, J=5.6 Hz, 2H).

Step 2. To a solution of 2-[2-[2-[2-[2-[2-(2-aminoethoxy)ethoxy]ethoxy]ethoxy]ethoxy]ethoxy]ethanol (1.3 g, 2.6 mmol, 1.0 equiv) in MeOH (5 mL) was added Pd/C (1.0 g, 10% purity) at 25° C. under N₂, the mixture was stirred at 25° C. for 16 h under H2. The mixture was filtered and concentrated to afford 4-amino-N-(20-hydroxy-3,6,9,12, 15,18-hexaoxaicosyl) benzenesulfonamide (1.0 g, crude) as a colorless oil.

Step 3. To a solution of 4-amino-N-[2-[2-[2-[2-[2-[2-(2-hydroxyethoxy)ethoxy]ethoxy]ethoxy]ethoxy]ethoxy]ethyl]benzenesulfonamide (500 mg, 1.0 mmol, 1.0 equiv) and 2-[(5-bromo-2-chloro-pyrimidin-4-yl)amino]-6-fluoro-benzamide (359 mg, 1. mmol, 1.0 equiv) in i-PrOH (5 mL) was added HCl (38 mg, 377 μmol, 36% purity, 0.4 equiv). The mixture was stirred at 90° C. for 16 h. The reaction mixture was filtered and concentrated under reduced to afford 2-[[5-bromo-2-[4-[2-[2-[2 [2 [2 [2 (2hydroxyethoxy)ethoxy]ethoxy]ethoxy]ethoxy]ethoxy]ethylsulfamoyl]anilino]pyrimidin-4-yl]amino]-6-fluoro-benzamide (500 mg, 613 μmol, 59% yield) as a yellow oil. ¹H NMR: (400 MHZ, DMSO-d₆) δ=10.16 (s, 1H), 10.00 (s, 1H), 8.40 (s, 1H), 8.25-8.06 (m, 3H), 7.83 (d, J=8.8 Hz, 2H), 7.64 (d, J=8.8 Hz, 2H), 7.57-7.48 (m, 2H), 7.10 (t, J=9.2 Hz, 1H), 3.60-3.30 (m, 26H), 3.16 (s, 1H), 2.86 (d, J=4.0 Hz, 2H).

Step 4. To a solution of 2-[5-bromo-2-[4-[2-[2-[2-[2-[2-[2-(2-hydroxyethoxy) ethoxy]ethoxy]ethoxy]ethoxy]ethoxy]ethylsulfamoyl]anilino]pyrimidin-4-yl]amino]-6-fluoro-benzamide (280 mg, 354 μmol, 1.0 equiv) in DMF (5 mL) was added DMP (451 mg, 1.0 mmol, 329 μL, 3.0 equiv). The mixture was stirred at 20° C. for 2 h. The reaction mixture was used into the next step without further purification.

Step 5. A mixture of 2-[[5-bromo-2-[4-[2-[2-[2-[2-[2-[2-(2-oxoethoxy)ethoxy]ethoxy]ethoxy]ethoxy]ethoxy]ethylsulfamoyl]anilino]pyrimidin-4-yl]amino]-6-fluoro-benzamide (280 mg, 355 μmol, 1.0 equiv), tert-butyl(E)-4-(methylamino)but-2-enoate (243 mg, 1.4 mmol, 4.0 equiv), TEA (359 mg, 3.6 mmol, 495 μL, 10.0 equiv) in DCM (10 mL) was added NaBH (OAc)3 (753 mg, 3.6 mmol, 10.0 equiv) at 0° C., and then the mixture was stirred at 20° C. for 0.5 h. The reaction mixture was quenched by addition sat. NaHCO₃ (30 mL) at 20° C. The combined organic layers were washed with DCM (20 mL×3), dried over Na₂SO₄. concentrated under reduced pressure to give a residue. The residue was purified by prep-HPLC (column: YMC Triart C18 250*50 mm*7 μm; mobile phase: [water (NH₄HCO₃)-ACN]; B %: 52%-82%, 8 min) to afford tert-butyl(E)-4-[2-[2-[2-[2-[2-[2-[2-[[4-[[5-bromo-4-(2-carbamoyl-3fluoroanilino) pyrimidin2yl]amino]phenyl]sulfonylamino]ethoxy]ethoxy]ethoxy]ethoxy]ethoxy]ethoxy]ethyl-methyl-amino]but-2-enoate (100 mg, 95 μmol, 27% yield) as white solid. ¹H NMR: (400 MHZ, CD₃OD) δ 8.36 (d, J=8.8 Hz, 1H), 8.28 (s, 1H), 8.19 (s, 1H), 7.88-7.80 (m, 2H), 7.77-7.68 (m, 2H), 7.51 (t, J=6.6, 8.4 Hz, 1H), 7.00 (d, J=8.4, 10.4 Hz, 1H), 6.81 (t, J=6.6, 15.6 Hz, 1H), 5.91 (d, J=15.6 Hz, 1H), 3.66-3.41 (m, 26H), 3.20 (d, J=5.4 Hz, 2H), 3.04 (t, J=5.4 Hz, 2H), 2.59 (t, J=5.4 Hz, 2H), 2.26 (s, 3H), 1.46 (s, 9H).

Step 6. To a solution of tert-butyl(E)-4-[2-[2-[2-[2-[2-[2-[2-[[4-[[5-bromo-4-(2-carbamoyl-3-fluoro-anilino) pyrimidin-2-yl]amino]phenyl]sulfonylamino]ethoxy]ethoxy]ethoxy]ethoxy]ethoxy]ethoxy]ethyl-methyl-amino]but-2-enoate (120 mg, 127 μmol, 1.0 equiv) in DCM (2 mL) was added TFA (218 mg, 1.9 mol, 141 μL, 15.0 equiv). The mixture was stirred at 20° C. for 0.5 h. The reaction mixture was concentrated under reduced pressure to afford (E)-4-[2-[2-[2-[2-[2-[2-[2-[[4-[[5-bromo-4-(2-carbamoyl-3-fluoro-anilino) pyrimidin-2-yl]amino]phenyl]sulfonylamino]ethoxy]ethoxy]ethoxy]ethoxy]ethoxy]ethoxy]ethyl-methyl-amino]but-2-enoic acid (120 mg, crude, TFA) as a brown oil and it was used into the next step without further purification.

Step 7. To a solution of (E)-4-[2-[2-[2-[2-[2-[2-[2-[[4-[[5-bromo-4-(2-carbamoyl-3-fluoro-anilino) pyrimidin-2-yl]amino]phenyl]sulfonylamino]ethoxy]ethoxy]ethoxy]ethoxy]ethoxy]ethoxy]ethyl-methyl-amino]but-2-enoic acid (120 mg, 127 μmol, 1.0 equiv, TFA) and 3-(4-phenoxyphenyl)-1-[(3R)-3-piperidyl]pyrazolo[3,4-d]pyrimidin-4-amine (64 mg, 127 μmol, 1.0 equiv, TFA) in DMF (2 mL) was added DIEA (64 mg, 636 μmol, 110 μL, 5.0 equiv) and HATU (96 mg, 252 μmol, 2.0 equiv). The mixture was stirred at 20° C. for 15 min. The residue was purified by prep-HPLC (column: Phenomenex luna C18 150*25 mm*10 μm; mobile phase: [water (FA)-ACN]; B %: 25%-55%, 10.5 min) to afford 2-[[2-[4-[2-[2-[2-[2-[2-[2-[2-[[(E)-4-[(3R)-3-[4-amino-3-(4-phenoxyphenyl) pyrazolo[3,4-d]pyrimidin-1-yl]-1-piperidyl]-4-oxo-but-2-enyl]-methyl-amino]ethoxy]ethoxy]ethoxy]ethoxy]ethoxy]ethoxy]ethylsulfamoyl]anilino]-5-bromo-pyrimidin-4-yl]amino]-6-fluoro-benzamide (50 mg, 39 μmol, 49% yield, 98% purity) as white solid. ¹H NMR: (400 MHZ, DMSO-d₆) δ=10.12 (s, 1H), 9.93 (s, 1H), 8.51-7.99 (m, 5H), 7.84 (d, J=8.4 Hz, 2H), 7.73-7.58 (m, 4H), 7.56-7.37 (m, 4H), 7.26-7.02 (m, 6H), 6.94 (d, J=15.6 Hz, 1H), 6.83 (d, J=14.4 Hz, 1H), 6.70-6.52 (m, 1H), 4.82-4.64 (m, 1H), 4.60-4.48 (m, 1H), 4.32-4.10 (m, 1H), 4.09-3.98 (m, 1H), 3.91-3.41 (m, 30H), 2.86 (d, J=5.6 Hz, 2H), 2.74-2.60 (m, 3H), 2.35-2.20 (m, 1H), 2.12 (d, J=8.4 Hz, 1H), 1.93 (d, J=1.6 Hz, 1H), 1.71-1.48 (m, 1H). LC-MS: MS (ES⁺): RT=2.602 min, m/z=1256.2 [M+2H⁺], LCMS method 05.

### Example 45—the Synthetic Route for I-66

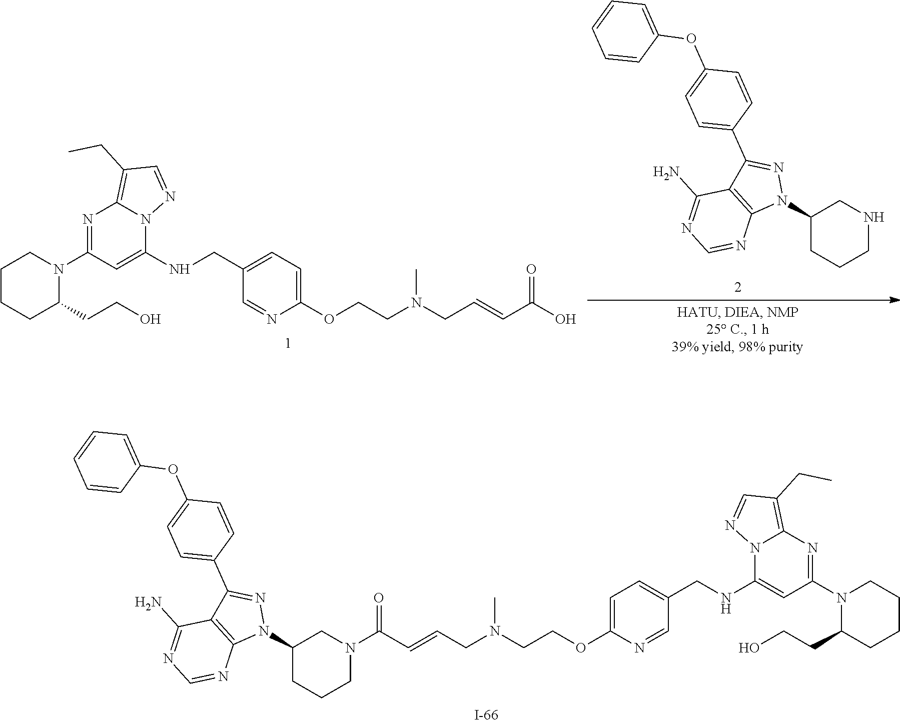

General Information: Synthetic route for compound 1 is described below. Synthetic route for compound 2 is described in WO2017/39425.

Step 1. To a solution of (E)-4-[2-[5-[[3-ethyl-5-[(2S)-2-(2-hydroxyethyl)-1-piperidyl]pyrazolo[1,5-a]pyrimidin-7-yl]amino]methyl]-2-pyridyl]oxy]ethyl-methyl-amino]but-2-enoic acid (50 mg, 93 μmol, 1.0 equiv) and 3-(4-phenoxyphenyl)-1-[(3R)-3-piperidyl]pyrazolo[3,4-d]pyrimidin-4-amine (108 mg, 279 μmol, 3.0 equiv) in NMP (2 mL) was added DIEA (36 mg, 279 μmol, 4 μL, 3.0 equiv) and HATU (42 mg, 112 μmol, 1.2 equiv). The mixture was stirred at 25° C. for 1 h. The reaction mixture was concentrated under reduced pressure to remove solvent. The residue was purified by the prep-HPLC (column: Phenomenex C18 150*25 mm*10 μm; mobile phase: [water (NH₄HCO₃)-ACN]; B %: 50%-80%,5 min) to give the (E)-1-[(3R)-3-[4-amino-3-(4-phenoxyphenyl) pyrazolo[3,4-d]pyrimidin-1-yl]-1-piperidyl]-4-[2-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethyl-methyl-amino]but-2-en-1-one (33 mg, 36 μmol, 39% yield, 98% purity) as a white solid. ¹H NMR: (400 MHZ, MeOH)8=7.93-8.19 (m, 2H), 7.43-7.72 (m, 4H), 7.20-7.36 (m, 2H), 6.90-7.11 (m, 5H), 6.38-6.77 (m, 3H), 5.41 (s, 1H), 4.26-4.50 (m, 4H), 3.73-4.23 (m, 5H), 3.23-3.48 (m, 4H), 3.04-3.10 (m, 1H), 2.83-2.97 (m, 1H), 2.51-2.80 (m, 2H), 2.36-2.50 (m, 2H), 1.83-2.34 (m, 7H), 1.20-1.77 (m, 9H), 1.06-1.16 (m, 3H). LC-MS: MS (ES⁺): RT=2.368 min, m/z=906.7 [M+H⁺]; LCMS method 05.

### Synthesis of 1:

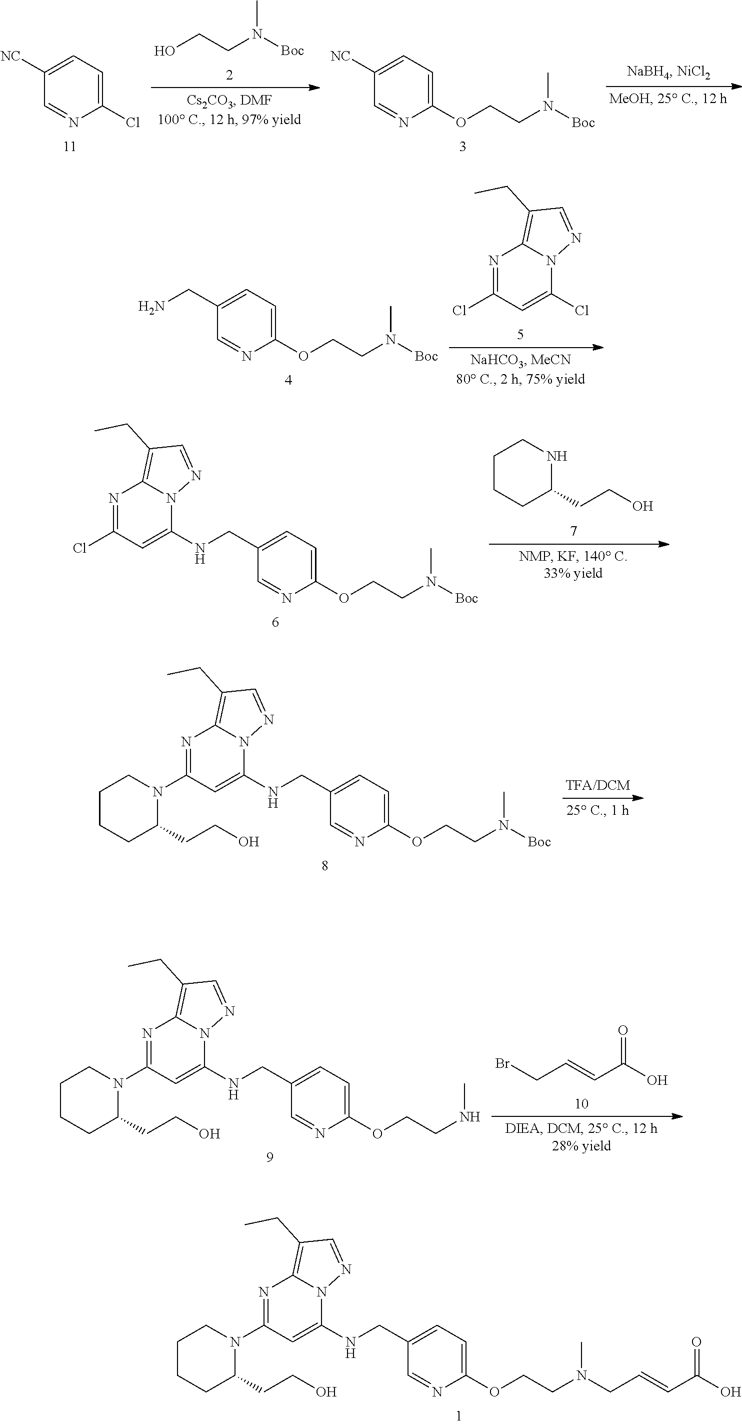

Step 1. A mixture of 6-chloropyridine-3-carbonitrile (2.0 g, 14.4 mmol, 1.0 equiv), tert-butyl N-(2-hydroxyethyl)-N-methyl-carbamate (3.0 g, 17.3 mmol, 1.2 equiv), Cs₂CO₃ (9.4 g, 28.8 mmol, 2.0 equiv) in DMF (20 mL) was degassed and purged with N₂ for 3 times and then the mixture was stirred at 100° C. for 12 h under N₂ atmosphere. The mixture was poured into H₂O (20 mL) and extracted with EtOAc (50 mL×2). The combined organic layers were washed with brine (100 mL), dried, filtered and concentrated to give a residue. The residue was purified by column chromatography (SiO₂, Petroleum ether/Ethyl acetate=10/1 to 5/1) to give tert-butyl N-[2-[(5-cyano-2-pyridyl)oxy]ethyl]-N-methyl-carbamate (3.9 g, 14.1 mmol, 97.4% yield) as a colorless oil ¹H NMR: (400 MHZ, CDCl₃)8=8.46 (d, J=2.4 Hz, 1H), 7.82-7.72 (m, 1H), 6.80 (d, J=8.6 Hz, 1H), 4.50 (s, 2H), 3.60 (s, 2H), 2.92 (s, 3H), 1.42 (s, 9H).

Step 2. To a mixture of tert-butyl N-[2-[(5-cyano-2-pyridyl)oxy]ethyl]-N-methyl-carbamate (3.9 g, 14.1 mmol, 1.0 equiv), NiCl₂·6H₂O (660 mg, 0.28 mmol, 0.2 equiv) in MeOH (50 mL) stirred at 0° C. under N₂ protection was added NaBH₄ (1.06 g, 28.2 mmol, 2.0 equiv), then the mixture was stirred at 25° C. for 12 h under N₂ protection. The mixture was poured into saturated NH₄Cl (100 mL) and extracted with DCM (100 mL×3). The mixture was washed with brine (100 mL), dried over Na₂SO₄, filtered and concentrated to give the residue and used for next step without further purification.

Step 3. A mixture of tert-butyl N-[2-[[5-(aminomethyl)-2-pyridyl]oxy]ethyl]-N-methyl-carbamate (1.2 g, 4.2 mmol, 1.2 equiv), 5,7-dichloro-3-ethyl-pyrazolo[1,5-a]pyrimidine (750 mg, 3.5 mmol, 1.0 equiv), DIPEA (448 mg, 3.5 mmol, 604 μL, 1.0 equiv), NaHCO₃ (1.5 g, 17.4 mmol, 675 μL, 5.0 equiv) in CH₃CN(10 mL) was degassed and purged with N₂ for 3 times and then the mixture was stirred at 80° C. for 2 h under N₂ atmosphere. The mixture was filtered and concentrated to give the residue. The residue was purified by column chromatography (SiO₂, Petroleum ether/Ethyl acetate=10/1 to 5/1). Tert-butyl N-[2-[[5-[[(5-chloro-3-ethyl-pyrazolo[1,5-a]pyrimidin-7-yl)amino]methyl]-2-pyridyl]oxy]ethyl]-N-methyl-carbamate (1.2 g, 2.6 mmol, 75% yield) was obtained as a colorless oil. ¹H NMR: (400 MHZ, CDCl₃) δ=8.15 (d, J=2.4 Hz, 1H), 7.85 (s, 1H), 7.64-7.54 (m, 1H), 6.76 (d, J=8.6 Hz, 1H), 6.69-6.60 (m, 1H), 5.93 (s, 1H), 4.50 (d, J=5.8 Hz, 2H), 4.44 (s, 2H), 3.60 (s, 2H), 2.95 (s, 3H), 2.86-2.72 (m, 2H), 1.44 (s, 9H), 1.32-1.27 (m, 3H).

Step 4. A mixture of tert-butyl N-[2-[[5-[[(5-chloro-3-ethyl-pyrazolo[1,5-a]pyrimidin-7-yl)amino]methyl]-2-pyridyl]oxy]ethyl]-N-methyl-carbamate (500 mg, 1.1 mmol, 1.0 equiv), 2-[(2S)-2-piperidyl]ethanol (210 mg, 1.6 mmol, 1.5 equiv), KF (315 mg, 5.4 mmol, 127, 5.0 equiv) in NMP (1 mL) was degassed and purged with N₂ for 3 times, and then the mixture was stirred at 140° C. for 36 h under N₂ atmosphere. The mixture was poured into H₂O (10 mL) and extracted with DCM (30 mL×2). The organic layer was concentrated to give the residue. The residue was purified by column chromatography (SiO₂, Petroleum ether/Ethyl acetate=10/1 to 3/1) to give the tert-butylN-[2-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethyl]-N-methyl-carbamate (200 mg, 361 μmol, 33% yield). ¹H NMR: (400 MHZ, CDCl₃) δ=8.10 (d, J=2.1 Hz, 1H), 7.58-7.51 (m, 2H), 6.60 (d, J=8.5 Hz, 1H), 6.31-6.15 (m, 1H), 5.23 (s, 1H), 5.09-4.92 (m, 1H), 4.42-4.31 (m, 4H), 3.63 (d, J=14.8 Hz, 1H), 3.59-3.57 (m, 1H), 3.56-3.46 (m, 4H), 3.03-2.90 (m, 2H), 2.88 (s, 3H), 2.57-2.49 (m, 2H), 2.19-2.07 (m, 2H), 1.65-1.58 (m, 3H), 1.36 (s, 9H), 1.23-1.16 (m, 4H).

Step 5. To a solution of tert-butyl N-[2-[[5-[[3-ethyl-5-[(2S)-2-(2-hydroxyethyl)-1-piperidyl]pyrazolo[1,5-a]pyrimidin-7-yl]amino]methyl]-2-pyridyl]oxy]ethyl]-N-methyl-carbamate (20 mg, 30 μmol, 1.0 equiv) in DCM (0.75 mL), TFA (0.3 mL) was stirred at 25° C. for 1 h. The mixture was concentrated to give the residue. The residue was used for next step without further purification. 2-[(2S)-1-[3-ethyl-7-[[6-[2-(methylamino)ethoxy]-3-pyridyl]methylamino]pyrazolo[1,5-a]pyrimidin-5-yl]-2-piperidyl]ethanol (13.59 mg, 29.96 μmol) was obtained as a colorless oil.

Step 6. To a solution of 2-[(2S)-1-[3-ethyl-7-[[6-[2-(methylamino)ethoxy]-3-pyridyl]methylamino]pyrazolo[1,5-a]pyrimidin-5-yl]-2-piperidyl]ethanol (1.0 g, 1.7 mmol, 1.0 equiv, TFA) in THF (10 mL) was added (E)-4-bromobut-2-enoic acid (233 mg, 1.4 mmol, 33 μL, 0.8 equiv) and DIEA (683 mg, 5.3 mmol, 921 μL, 3 equiv). The mixture was stirred at 25° C. for 12 h. The reaction mixture was concentrated under reduced pressure to remove solvent. The residue was purified by the prep-HPLC (column: Phenomenex C18 250*50 mm*10 μm; mobile phase: [water (NH₄HCO₃)-ACN]; B %: 17%-47%, 8 min) to give the (E)-4-[2-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethyl-methyl-amino]but-2-enoic acid (270 mg, 497 μmol, 28% yield) as a white solid. ¹H NMR: (400 MHZ, CDCl₃) δ=8.13 (d, J=2.0 Hz, 1H), 7.55-7.72 (m, 2H), 6.91 (d, J-8.4 Hz, 2H), 6.31-6.53 (m, 1H), 5.72 (d, J=16.0 Hz, 1H), 5.07-5.24 (m, 1H), 4.41-4.64 (m, 2H), 4.32 (d, J=4.0 Hz, 2H), 3.52-3.92 (m, 2H), 3.25-3.48 (m, 1H), 2.95-3.18 (m, 3H), 2.67-2.83 (m, 2H), 2.56-2.64 (m, 2H), 2.45 (s, 3H), 2.09-2.20 (m, 1H), 1.64-1.85 (m, 9H), 1.15-1.30 (m, 3H).

### Example 46—the Synthetic Route for I-67

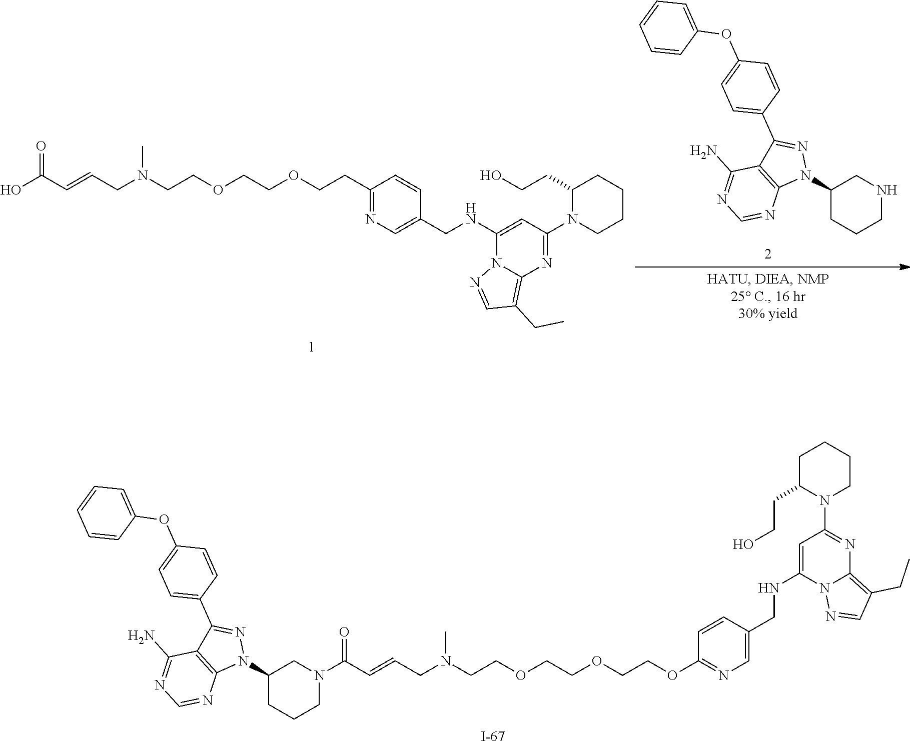

General Information: Synthetic route for compound 2 is described in WO2017/39425.

Step 1. To a solution of (R)-3-(4-phenoxyphenyl)-1-(piperidin-3-yl)-1H-pyrazolo[3,4-d]pyrimidin-4-amine (18 mg, 46 μmol, 1.0 equiv) in DMF (1 mL) was added HATU (18 mg, 46 μmol, 1.0 equiv) and DIEA (18 mg, 139 μmol, 24 μL, 3.0 equiv), (S,E)-4-((2-(2-(2-(5-(((3-ethyl-5-(2-(2-hydroxyethyl) piperidin-1-yl) pyrazolo[1,5-a]pyrimidin-7-yl)amino) methyl) pyridin-2-yl)ethoxy)ethoxy) ethyl)(methyl)amino)but-2-enoic acid (29 mg, 46 μmol, 1.0 equiv). The mixture was stirred at 25° C. for 16 h to give a yellow solution. The reaction mixture was diluted with water 30 mL and extracted with EA mL (20 mL×3). The combined organic layers were washed with water (25 mL×2), dried over Na₂SO₄, filtered and concentrated under reduced pressure to give a residue. The crude product was purified by reversed-phase HPLC column: Phenomenex C18 150*25 mm*10 μm; mobile phase: [water (NH₄HCO₃)-ACN]; B %: 46%-76%, 8 min. Compound (E)-1-((R)-3-(4-amino-3-(4-phenoxyphenyl)-1H-pyrazolo[3,4-d]pyrimidin-1-yl) piperidin-1-yl)-4-((2-(2-(2-(5-(((3-ethyl-5-((S)-2-(2-hydroxyethyl) piperidin-1-yl) pyrazolo[1,5-a]pyrimidin-7-yl)amino) methyl) pyridin-2-yl)ethoxy)ethoxy) ethyl)(methyl)amino)but-2-en-1-one (14 mg, 14 μmol, 30% yield) was obtained as a white solid. ¹H NMR: (400 MHZ, CD₃OD) δ=8.29-8.19 (m, 2H), 7.84-7.75 (m, 2H), 7.71-7.65 (m, 2H), 7.46-7.38 (m, 2H), 7.22-7.13 (m, 3H), 7.12-7.07 (m, 2H), 6.88-6.77 (m, 1H), 6.73-6.57 (m, 1H), 5.73-5.64 (m, 1H), 4.70-4.59 (m, 3H), 4.50-4.41 (m, 2H), 4.41-4.34 (m, 1H), 4.27-4.18 (m, 1H), 3.92-3.91 (d, J=9.2 Hz, 4H), 3.90-3.82 (m, 3H), 3.81-3.72 (m, 5H), 3.71-3.60 (m, 3H), 3.59-3.34 (m, 2H), 3.29 (s, 2H), 2.93-2.85 (m, 3H), 2.64-2.54 (m, 2H), 2.38-2.28 (m, 1H), 2.24-2.14 (m, 2H), 2.10-1.98 (m, 1H), 1.91-1.60 (m, 9H), 1.32 (m, 4H). LC-MS: MS (ES⁺): RT=2.535 min, m/z=994.5 [M+H⁺], LCMS method: LCMS method 05.

### Synthesis of Compound 1

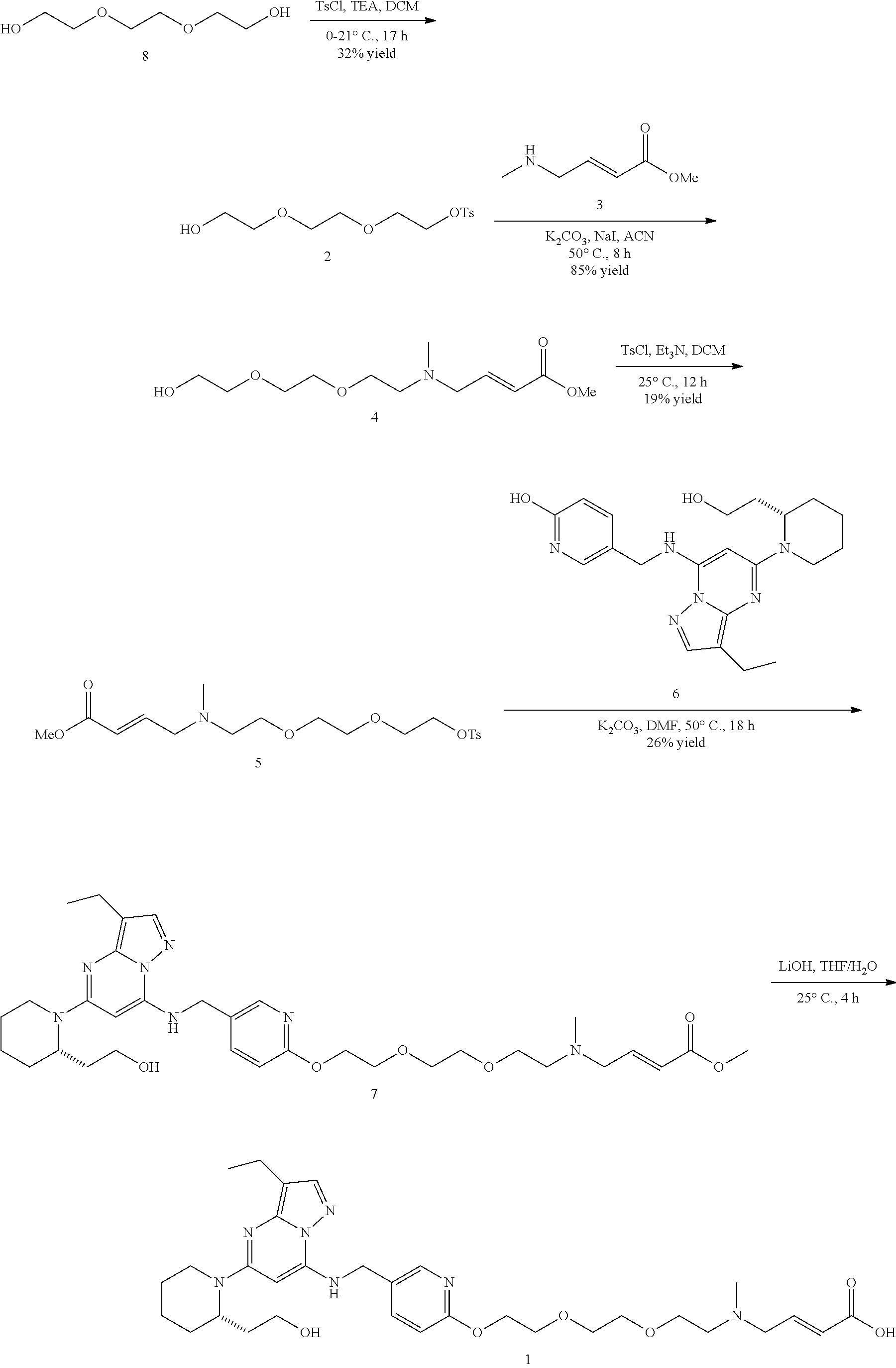

General Information: Synthetic route for compound 3 is described in WO2017/100668.

Step 1. To a solution of 2-[2-(2-hydroxyethoxy)ethoxy]ethanol (20.0 g, 133 mmol, 18 mL, 1.0 equiv) and Et₃N(40.4 g, 400 mmol, 56 mL, 3.0 equiv) in DCM (200 mL) was stirred at 0° C. for 1 h, then the TosCl (25.4 g, 133 mmol, 1.0 equiv) in DCM (100 mL) was added to the mixture and stirred at 21° C. for 16 h. The reaction mixture was filtered and concentrated under reduced pressure to give a residue. The residue was purified by flash silica gel chromatography (ISCO®; 330 g SepaFlash® Silica Flash Column, Eluent of 5˜50% Ethylacetate/Petroleum ethergradient at 100 mL/min) to afford 2-(2-(2-hydroxyethoxy)ethoxy) ethyl 4-methyl benzenesulfonate (13.0 g, 42.0 mmol, 32% yield,) as a light yellow liquid.

Step 2. To a solution of 2-(2-(2-hydroxyethoxy)ethoxy) ethyl 4-methyl benzenesulfonate (1.5 g, 4.9 mmol, 1.0 equiv) in ACN(20 mL) was added K₂CO₃ (2.1 g, 14.8 mmol, 3.0 equiv), NaI (738 mg, 4.9 mmol, 1.0 equiv), (E)-methyl 4-(methylamino)but-2-enoate (636 mg, 4.9 mmol, 1.0 equiv). The mixture was stirred at 50° C. for 8 h. The reaction mixture was partitioned between H₂O (100 mL) and EA (100 mL). The organic phase was separated, washed with water (50 mL×3), dried over Na₂SO₄, filtered and concentrated under reduced pressure to give a residue. The residue was purified by column chromatography (SiO₂, DCM: MeOH=20/1 to 10/1). Compound (E)-methyl 4-((2-(2-(2-hydroxyethoxy)ethoxy) ethyl)(methyl)amino)but-2-enoate (1.10 g, 4.21 mmol, 85% yield) was obtained as a light yellow oil. ¹H NMR: (400 MHZ, CDCl₃) δ=7.00-6.78 (t, J=15.6 Hz, 6.4 Hz, 1H), 6.00-5.82 (d, J=15.6 Hz, 1H), 3.67 (s, 3H), 3.66-3.49 (m, 10H), 3.16 (d, J=6.4 Hz, 2H), 2.55 (t, J=5.6 Hz, 2H), 2.23 (s, 3H).

Step 3. To a solution of (E)-methyl 4-((2-(2-(2-hydroxyethoxy)ethoxy) ethyl)(methyl)amino)but-2-enoate (1.0 g, 3.8 mmol, 1.0 equiv) in DCM (10 mL) was added TsCl (540 mg, 7.6 mmol, 2.0 equiv) and TEA (1.2 g, 11.5 mmol, 1.6 mL, 3.0 equiv). The mixture was stirred at 25° C. for 12 h. The reaction mixture was concentrated under reduced pressure to remove solvent. The residue was diluted with water 50 mL and extracted with EA (50 mL×3). The combined organic layers were washed with water (50 mL×2), dried over Na₂SO₄ filtered and concentrated under reduced pressure to give a residue. The residue was purified by column chromatography (SiO₂, DCM/MeOH=20/1 to 10/1) to give (E)-methyl 4-(methyl (2-(2-(2-(tosyloxy)ethoxy)ethoxy) ethyl)amino)but-2-enoate (300 mg, 722 μmol, 19% yield) was obtained as a yellow oil. ¹H NMR: (400 MHZ, CDCl₃) δ=7.76-7.70 (d, J=8.4 Hz, 2H), 7.30-7.25 (d, J=8.0 Hz, 2H), 6.89 (m, 1H), 5.94-5.90 (d, J=15.6 Hz, 1.2 Hz, 1H), 4.12-4.05 (m, 2H), 3.68-3.65 (m, 3H), 3.64-3.61 (m, 2H), 3.57-3.54 (m, 2H), 3.51 (d, J=1.7, 3.4 Hz, 2H), 3.49-3.47 (m, 2H), 3.15-3.11 (m, 2H), 2.55-2.48 (m, 2H), 2.36 (m, 3H), 2.23-2.16 (s, 3H).

Step 4. To a solution of (E)-methyl 4-(methyl (2-(2-(2-(tosyloxy)ethoxy)ethoxy) ethyl)amino)but-2-enoate (150 mg, 361 μmol, 1.5 equiv) in DMF (1.0 mL) was added K₂CO₃ (100 mg, 722 μmol, 3.0 equiv) and(S)-5-(((3-ethyl-5-(2-(2-hydroxyethyl) piperidin-1-yl) pyrazolo[1,5-a]pyrimidin-7-yl)amino) methyl) pyridin-2-ol (95 mg, 241 μmol, 1.0 equiv). The mixture was stirred at 50° C. for 18 h. The reaction mixture was diluted with H₂O (20 mL) and extracted with EA (20 mL×3). The combined organic layers were washed with brine (25 mL×2), dried over Na₂SO₄, filtered and concentrated under reduced pressure to give a residue. The crude product was purified by reversed-phase HPLC (column: Waters xbridge 150*25 mm 10 μm; mobile phase: [water (NH₄HCO₃)-ACN]; B %: 40%-70%,11 min) to give methyl (S,E)-methyl 4-((2-(2-(2-(5-(((3-ethyl-5-(2-(2-hydroxyethyl) piperidin-1-yl) pyrazolo[1,5-a]pyrimidin-7-yl)amino) methyl) pyridin-2-yl)ethoxy)ethoxy) ethyl)(methyl)amino)but-2-enoate (40 mg, 62 μmol, 26% yield) as a light yellow oil. ¹H NMR: (400 MHZ, CDCl₃) δ=8.10-8.04 (d, J=2.4 Hz, 1H), 7.57-7.50 (m, 2H), 6.94-6.85 (m, 1H), 6.75-6.69 (d, J=8.4 Hz, 1H), 6.24-6.15 (m, 1H), 5.94-5.93 (m, 1H), 5.22 (s, 1H), 5.07-4.99 (m, 1H), 4.43-4.39 (m, 2H), 4.38-4.33 (m, 2H), 3.80-3.77 (m, 2H), 3.66-3.65 (m, 3H), 3.65-3.63 (m, 2H), 3.59-3.56 (m, 2H), 3.56-3.50 (m, 4H), 3.30-3.23 (m, 1H), 3.15-3.12 (m, 2H), 3.01-2.93 (m, 1H), 2.56-2.51 (m, 4H), 2.21 (s, 3H), 2.05-1.97 (m, 1H), 1.60 (s, 4H), 1.20-1.16 (m, 7H).

Step 5. To a solution of (S,E)-methyl 4-((2-(2-(2-(5-(((3-ethyl-5-(2-(2-hydroxyethyl) piperidin-1-yl) pyrazolo[1,5-a]pyrimidin-7-yl)amino) methyl) pyridin-2-yl)ethoxy)ethoxy) ethyl)(methyl)amino)but-2-enoate (30 mg, 46 μmol, 1.0 equiv) in THF (1 mL) and H₂O (0.3 mL), was added LiOH·H2O (2.0 mg, 47 μmol, 1.0 equiv). The mixture was stirred at 25° C. for 4 h. The reaction mixture was quenched by addition H₂O (20 mL), added HCl (1 mol) to the mixture, ended the pH to 8, and extracted with EA (20 mL×3). The combined organic layers were washed with water (20 mL×3), dried over Na₂SO₄, filtered and concentrated under reduced pressure to give a residue. The residue was used for the next step without further purification. Compound (S,E)-4-((2-(2-(2-(5-(((3-ethyl-5-(2-(2-hydroxy ethyl) piperidin-1-yl) pyrazolo[1,5-a]pyrimidin-7-yl)amino) methyl) pyridin-2-yl)ethoxy)ethoxy) ethyl)(methyl)amino)but-2-enoic acid (27 mg, 43 μmol, crude) was obtained as a yellow oil.

### Example 47—the Synthetic Route for I-68

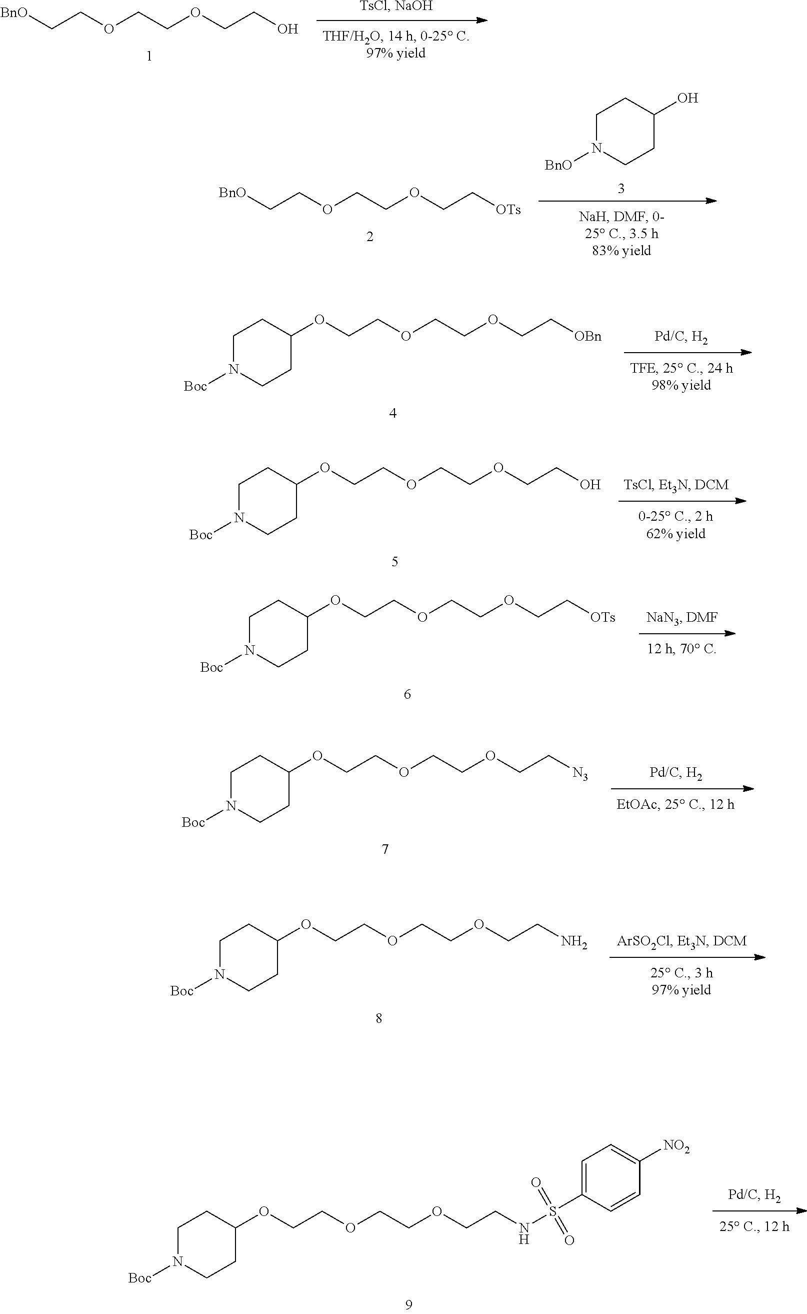

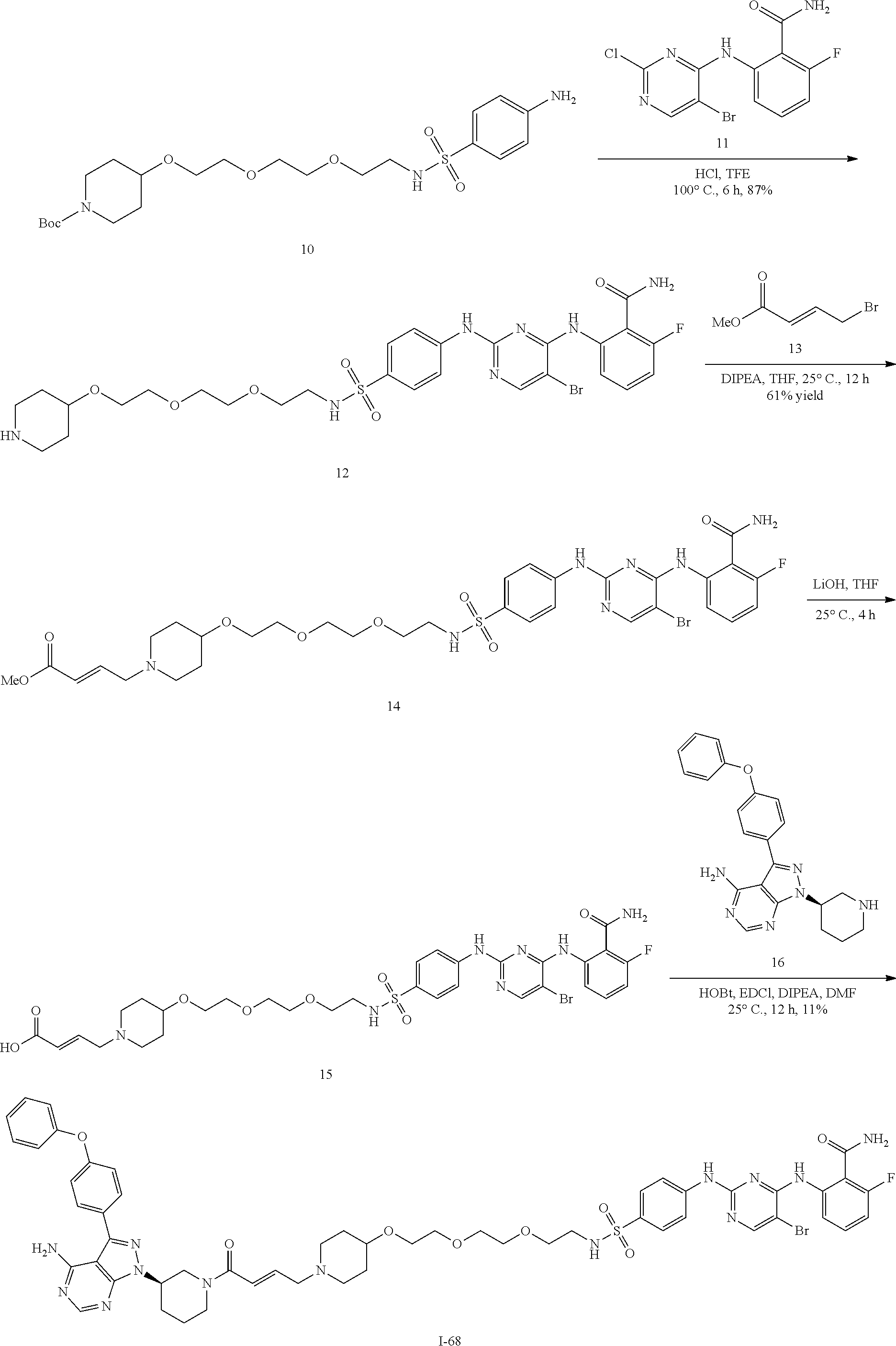

General Information: Synthetic route for compound 11 is described in Angewandte Chemie International Edition, 2020, 59, 13865. Synthetic route for compound 16 is described in WO2017/39425, 2017, A1.

Step 1. 2-[2-(2-benzyloxyethoxy)ethoxy]ethanol (25.0 g, 104 mmol, 1.0 equiv), NaOH (14.5 g, 364 mmol, 3.5 equiv) THF (50 mL) and H₂O (50 mL) were put to a three-necked flask, and the mixture was cooled at 0° C. To the mixture was added a solution of TsCl (11.9 g, 62.4 mmol, 1.2 equiv) in THF (100 mL) dropwise, and the reaction mixture was stirred for 2 h at 0° C. The reaction mixture was allowed to warm at 20° C., and stirred for additional 12 h. The reaction mixture was diluted with H₂O (300 mL) and extracted with DCM (300 mL×2). The combined organic layers were washed with aqueous brine (200 mL), dried over Na₂SO₄, filtered and concentrated under reduced pressure to give a residue. The residue was purified by flash silica gel chromatography (ISCO®; 80 g SepaFlash® Silica Flash Column, Eluent of 0˜50% Ethylacetate/Petroleum ether gradient @ 80 mL/min) to afford 2-[12-(2-benzyloxyethoxy)ethoxy]ethyl 4-methylbenzenesulfonate (40.0 g, 101 mmol, 97% yield, 100% purity) as a colorless oil. ¹H NMR: (400 MHZ, CDCl₃) δ 7.80-7.78 (m, 2H), 7.34-7.28 (m, 7H), 4.55 (s, 2H), 4.17-4.14 (m, 2H), 3.70-3.67 (m, 2H), 3.66-3.57 (m, 10H), 2.43 (s, 3H)

Step 2. To a solution of tert-butyl 4-hydroxypiperidine-1-carboxylate (2.8 g, 13.9 mmol, 1.1 equiv) in THF (50 mL) was added NaH (582 mg, 14.5 mmol, 60% purity, 1.1 equiv) at 0° C. Then it was stirred at 25° C. for 0.5 h. Then 2-[2-(2-benzyloxyethoxy)ethoxy]ethyl 4-methylbenzenesulfonate (5.0 g, 12.6 mmol, 1.0 equiv) was added to the mixture. The mixture was stirred at 25° C. for 3 h. The reaction mixture was quenched by addition (30 mL) NH₄Cl at 0° C., and then diluted with H₂O (30 mL) and extracted with EtOAc (50 mL×3). The combined organic layers were washed with brine (50 mL), dried over Na₂SO₄, filtered and concentrated under reduced pressure to give a residue. The residue was purified by flash silica gel chromatography (ISCO®; 40 g SepaFlash® Silica Flash Column, Eluent of 0˜50% Ethyl acetate/Petroleum ether gradient @ 40 mL/min) to afford tert-butyl 4-[2-[2-(2-benzyloxyethoxy)ethoxy]ethoxy]piperidine-1-carboxylate (4.5 g, 10.6 mmol, 83% yield) as a yellow oil. ¹H NMR: (400 MHZ, CDCl₃) δ 7.34-7.27 (m, 5H), 4.56 (s, 2H), 3.89-3.77 (m, 2H), 3.68-3.61 (m, 12H), 3.51-3.43 (m, 1H), 3.07-3.01 (m, 2H), 1.85-1.79 (m, 2H), 1.52-1.46 (m, 2H), 1.45 (s, 9H).

Step 3. A mixture of tert-butyl 4-[2-[2-(2-benzyloxyethoxy)ethoxy]ethoxy]piperidine-1-carboxylate (4.5 g, 10.6 mmol, 1.0 equiv), Pd/C (2.0 g, 10% purity) in CF₃CH₂OH (30 mL) was degassed and purged with H₂ for 3 times, and then the mixture was stirred at 25° C. for 24 h under H₂ atmosphere. The reaction mixture was filtered and concentrated under reduced pressure to afford tert-butyl 4-[2-[2-(2-hydroxyethoxy)ethoxy]ethoxy]piperidine-1-carboxylate (3.5 g, 10.5 mmol, 98% yield) as a light yellow oil. ¹H NMR: (400 MHZ, CDCl₃) δ=3.87-3.70 (m, 4H), 3.69-3.59 (m, 8H), 3.52-3.43 (m, 1H), 3.09-2.97 (m, 2H), 1.93-1.71 (m, 4H), 1.56-1.47 (m, 2H), 1.46-1.43 (m, 9H).

Step 4. To a solution of tert-butyl 4-[2-[2-(2-hydroxyethoxy)ethoxy]ethoxy]piperidine-1-carboxylate (2.5 g, 7.5 mmol, 1.0 equiv) in THF (20 mL) was added KOH (546 mg, 9.8 mmol, 1.3 equiv) and 4-methylbenzenesulfonyl chloride (1.7 g, 9.0 mmol, 1.2 equiv). The mixture was stirred at 20° C. for 2 h. The combined reaction mixture was diluted with H₂O (20 mL) and extracted with EtOAc (50 mL×3). The combined organic layers were washed with brine (50 mL), dried over Na₂SO₄, filtered and concentrated under reduced pressure to give a residue. Then residue was purified by flash silica gel chromatography (ISCO®; 40 g SepaFlash® Silica Flash Column, Eluent of 0˜50% Ethyl acetate/Petroleum ether gradient at 40 mL/min) to afford tert-butyl 4-[2-[2-[2-(p-tolylsulfonyloxy)ethoxy]ethoxy]ethoxy]piperidine-1-carboxylate (2.3 g, 4.7 mmol, 62% yield) as a colorless oil. ¹H NMR: (400 MHZ, CDCl₃) δ=7.79 (d, J=8.4 Hz, 2H), 7.34 (d, J=8.0 Hz, 2H), 4.17-4.14 (m, 2H), 3.76 (d, J=6.8, 5.6 Hz, 2H), 3.70-3.67 (m, 2H), 3.61-3.58 (m, 8H), 3.49-3.43 (m, 1H), 3.07-3.01 (m, 2H), 2.44 (s, 3H), 1.84-1.79 (m, 2H), 1.54-1.48 (m, 2H), 1.45 (s, 9H).

Step 5. To a solution of tert-butyl 4-[2-[2-[2-(p-tolylsulfonyloxy)ethoxy]ethoxy]ethoxy]piperidine-1-carboxylate (3.2 g, 6.6 mmol, 1.0 equiv) in DMF (30 mL) was added NaN3 (1.3 g, 19.2 mmol, 2.9 equiv) slowly at 0° C. The mixture was stirred at 70° C. for 12 h. The reaction mixture was diluted with H₂O (20 mL), and extracted with EtOAc (50 mL×3). The combined organic layers were washed with brine 50 mL, dried over Na₂SO₄, filtered and concentrated under reduced pressure to tert-butyl 4-[2-[2-(2-azidoethoxy)ethoxy]ethoxy]piperidine-1-carboxylate (2.0 g, 5.6 mmol, crude) as a yellow oil. ¹H NMR: (400 MHZ, CDCl₃) δ=3.91-3.71 (m, 3H), 3.71-3.59 (m, 9H), 3.48 (t, J=8.4, 4.0 Hz, 1H), 3.38 (t, J=5.6 Hz, 2H), 3.06 (d, J=13.2, 9.6, 3.6 Hz, 2H), 1.88-1.78 (m, 2H), 1.56-1.46 (m, 2H), 1.47 (s, 9H)

Step 6. A mixture of tert-butyl 4-[2-[2-(2-azidoethoxy)ethoxy]ethoxy]piperidine-1-carboxylate (2.0 g, 5.6 mmol, 1.0 equiv), Pd/C (200 mg, 10% purity) in MeOH (15 mL) was degassed and purged with H₂ for 3 times, and then the mixture was stirred at 25° C. for 12 h under H₂ atmosphere. The reaction mixture was filtered and concentrated under reduced pressure to afford tert-butyl 4-[2-[2-(2-aminoethoxy)ethoxy]ethoxy]piperidine-1-carboxylate (1.9 g, crude) as an colorless oil. ¹H NMR: (400 MHZ, CDCl₃) δ=3.90-3.71 (m, 3H), 3.68-3.60 (m, 8H), 3.54-3.44 (m, 3H), 3.10-2.98 (m, 3H), 2.89-2.84 (m, 2H), 1.86-1.80 (m, 2H), 1.53-1.49 (m, 2H), 1.45 (s, 9H).

Step 7. To a solution of tert-butyl 4-[2-[2-(2-aminoethoxy)ethoxy]ethoxy]piperidine-1-carboxylate (1.8 g, 5.5 mmol, 1.0 equiv) and 4-nitrobenzenesulfonyl chloride (1.2 g, 5.5 mmol, 1.0 equiv) in DCM (18 mL) was added Et₃N(1.1 g, 11.0 mmol, 1.5 mL, 2.0 equiv). The mixture was stirred at 25° C. for 3 h. The reaction mixture was diluted with H₂O (50 mL) and extracted with DCM (100 mL×2). The combined organic layers were washed with brine (200 mL), dried over Na₂SO₄, filtered and concentrated under reduced pressure to give a residue. The residue was purified by flash silica gel chromatography (ISCO®; 40 g SepaFlash® Silica Flash Column, Eluent of 0˜70% Ethyl acetate/Petroleum ether gradient at 40 mL/min) to afford tert-butyl 4-[2-[2-[2-[(4-nitrophenyl)sulfonylamino]ethoxy]ethoxy]ethoxy]piperidine-1-carboxylate (3.1 g, 5.7 mmol, 97% yield, 95% purity) as a yellow oil.

Step 8. A mixture of tert-butyl 4-[2-[2-[2-[(4-nitrophenyl)sulfonylamino]ethoxy]ethoxy]ethoxy]piperidine-1-carboxylate (3.0 g, 5.8 mmol, 1.0 equiv), Pd/C (300 mg, 10% purity) in EtOAc (30 mL) was degassed and purged with H₂ for 3 times, and then the mixture was stirred at 25° C. for 12 h under H₂ atmosphere. The reaction mixture was filtered and concentrated under reduced pressure to afford tert-butyl 4-[2-[2-(2-aminoethoxy)ethoxy]ethoxy]piperidine-1-carboxylate (1.9 g, crude) as a colorless oil. ¹H NMR: (400 MHZ, DMSO-d6) δ=7.47-7.37 (m, 2H), 7.12 (s, 1H), 6.65-6.55 (m, 2H), 5.90 (s, 2H), 3.62 (t, J=4.8, 13.2 Hz, 2H), 3.53-3.40 (m, 10H), 2.97 (d, J=9.6 Hz, 2H), 2.79 (d, J=3.6 Hz, 2H), 1.81-1.71 (m, 2H), 1.38 (s, 9H), 1.34-1.24 (m, 2H).

Step 9. To a solution of 2-[(5-bromo-2-chloro-pyrimidin-4-yl)amino]-6-fluoro-benzamide (268 mg, 778 μmol, 1.0 equiv) and tert-butyl 4-[2-[2-[2-[(4-aminophenyl) sulfonylamino]ethoxy]ethoxy]ethoxy]piperidine-1-carboxylate (400 mg, 820 μmol, 1.0 equiv) in TFE (60 mL) was added HCl (622 mg, 6.2 mmol, 610 μL, 36% purity, 7.5 equiv). The mixture was stirred at 100° C. for 6 h. The reaction mixture was concentrated under reduced pressure to remove TFE to give a residue. The residue was purified by prep-HPLC (column: Phenomenex Luna C18 200*40 mm*10 μm; mobile phase: [water (HCl)-ACN]; B %: 14%-44%, 10 min) to afford 2-[5-bromo-2-[4-[2-[2-[2-(4-piperidyloxy)ethoxy]ethoxy]ethylsulfamoyl]anilino]pyrimidin-4-yl]amino]-6-fluoro-benzamide (300 mg, 358 μmol, 87% yield) as a white solid. ¹H NMR: (400 MHZ, DMSO-d₆) δ=10.21-9.93 (m, 2H), 9.01-8.74 (m, 2H), 8.40 (d, J=1.2 Hz, 1H), 8.45-8.30 (m, 1H), 8.23-8.09 (m, 2H), 7.90-7.73 (m, 2H), 7.64 (d, J=8.0 Hz, 1H), 7.58-7.45 (m, 2H), 7.10 (t, J=9.2 Hz, 1H), 3.55-3.35 (m, 12H), 3.09 (s, 2H), 2.97-2.78 (m, 4H), 1.97-1.84 (m, 1H), 1.97-1.82 (m, 1H), 1.66 (br d, J=8.4 Hz, 2H).

Step 10. To a solution of methyl (E)-4-bromobut-2-enoate (41 mg, 230 μmol, 27 μL, 0.90 equiv) and 2-[[5-bromo-2-[4-[2-[2-[2-(4-piperidyloxy)ethoxy]ethoxy]ethylsulfamoyl]anilino]pyrimidin-4-yl]amino]-6-fluoro-benzamide (197 mg, 256 μmol, 1.0 equiv) in THF (2 mL) was added DIPEA (132 mg, 1.0 mmol, 178 μL, 4.0 equiv). The mixture was stirred at 25° C. for 12 h. The reaction mixture was concentrated under reduced pressure to give a residue. The residue was purified by prep-HPLC (column: Waters xbridge 150×25 mm 10 μm; mobile phase: [water (NH₄HCO₃)-ACN]; B %: 30%-60%, 8 min) to afford methyl (E)-4-[4-[2-[2-[2-[4-[[5-bromo-4-(2-carbamoyl-3-fluoro-anilino) pyrimidin-2-yl]amino]phenyl]sulfonylamino]ethoxy]ethoxy]ethoxy]-1-piperidyl]but-2-enoate (160 mg, 163 μmol, 63% yield) as a white solid.

Step 11. To a solution of methyl (E)-4-[4-[2-[2-[2-[[4-[[5-bromo-4-(2-carbamoyl-3-fluoro-anilino) pyrimidin-2-yl]amino]phenyl]sulfonylamino]ethoxy]ethoxy]ethoxy]-1-piperidyl]but-2-enoate (80 mg, 100 μmol, 1.0 equiv) in THF (2 mL) and H₂O (0.5 mL) was added LiOH·H₂O (13 mg, 302 μmol, 3.0 equiv). The mixture was stirred at 25° C. for 4 h. The reaction mixture was filtered and concentrated under reduced pressure to afford (E)-4-[4-[2-[2-[2-[4-[5-bromo-4-(2-carbamoyl-3-fluoro-anilino) pyrimidin-2-yl]amino]phenyl]sulfonylamino]ethoxy]ethoxy]ethoxy]-1-piperidyl]but-2-enoic acid (80 mg, crude) as a white solid.

Step 12. To a solution of (E)-4-[4-[2-[2-[2-[4-[[5-bromo-4-(2-carbamoyl-3-fluoro-anilino) pyrimidin-2-yl]amino]phenyl]sulfonylamino]ethoxy]ethoxy]ethoxy]-1-piperidyl]but-2-enoic acid (70 mg, 90 μmol, 1.0 equiv), (1R)-3-(4-phenoxyphenyl)-1-(3-piperidyl) pyrazolo[3,4-d]pyrimidin-4-amine (44 mg, 89 μmol, 1.0 equiv, TFA), HOBt (18 mg, 134 μmol, 1.5 equiv), and EDCI (26 mg, 134 μmol, 1.5 equiv) in DMF (2 mL) was added DIPEA (23 mg, 179 μmol, 31 UL, 2.0 equiv). The mixture was stirred at 25° C. for 12 h. The reaction mixture was poured into H₂O 20 mL and then extracted with EtOAc (2×20 mL). The organic layers were dried over anhydrous Na₂SO₄, filtered and concentrated to afford crude product. The residue was purified by prep-HPLC (column: Phenomenex Luna C18 100×30 mm×5 μm; mobile phase: [water (FA)-ACN]; B %: 22%-52%, 8 min) to afford 2-[[2-[4-[2-[2-[2-[[1-[(E)-4-[(3R)-3-[4-amino-3-(4-phenoxyphenyl) pyrazolo[3,4-d]pyrimidin-1-yl]-1-piperidyl]-4-oxo-but-2-enyl]-4-piperidyl]oxy]ethoxy]ethoxy]ethylsulfamoyl]anilino]-5-bromo-pyrimidin-4-yl]amino]-6-fluoro-benzamide (12 mg, 10 μmol, 11% yield, 98% purity) as a white solid. ¹H NMR: (400 MHz, CD₃OD) δ=8.48 (d, J=2.4 Hz, 1H), 8.35 (d, J=8.4 Hz, 1H), 8.23 (d, J=2.4 Hz, 2H), 7.85-7.75 (m, 2H), 7.74-7.59 (m, 4H), 7.51-7.33 (m, 4H), 7.17-7.05 (m, 4H), 6.97 (t, J=9.6 Hz, 1H), 6.85-6.67 (m, 1H), 6.60 (s, 1H), 4.20-4.01 (m, 2H), 3.96-3.88 (m, 1H), 3.61-3.56 (m, 5H), 3.48 (d, J=4.8, 6.4 Hz, 3H), 3.46-3.42 (m, 2H), 3.07-2.96 (m, 3H), 2.89-2.77 (m, 2H), 2.64-2.45 (m, 2H), 2.42-1.99 (m, 5H), 1.92-1.65 (m, 5H), 1.37-1.28 (m, 1H). LC-MS: MS (ES⁺): RT=2.731 min, m/z=556.3 [1/2M+H⁺], LCMS method 05.

### Example 48—the Synthetic Route for I-69

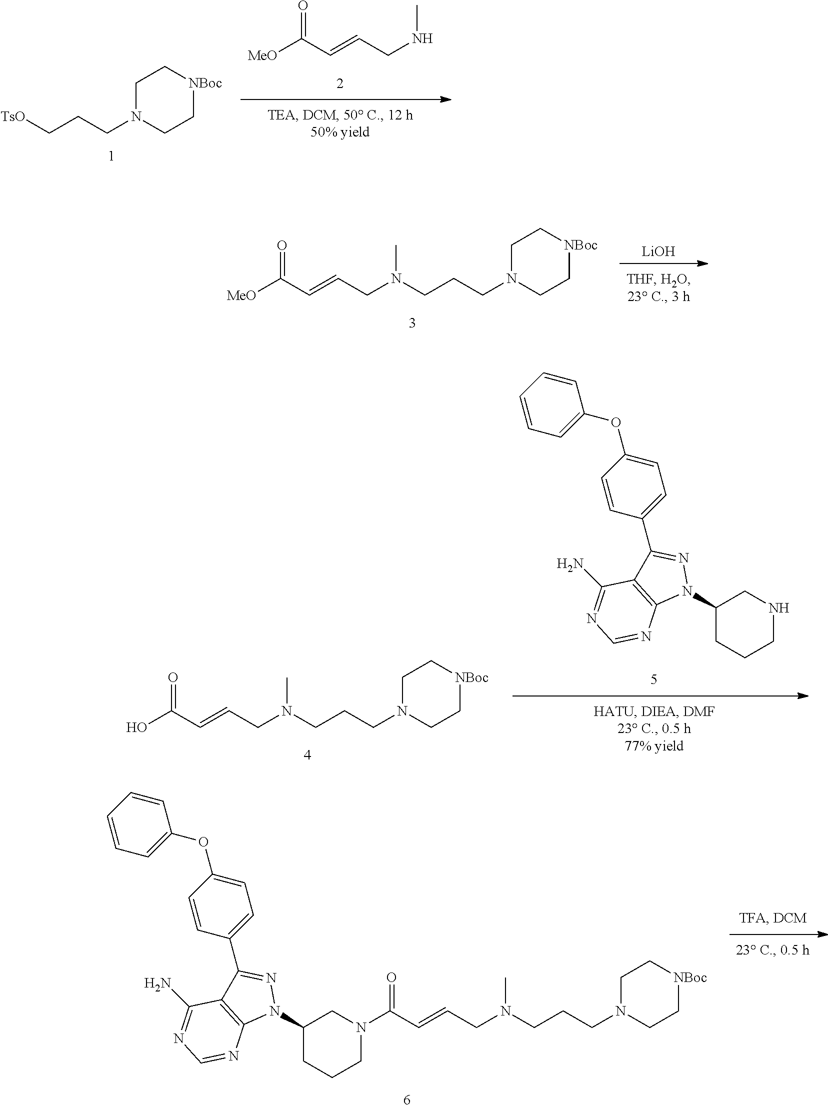

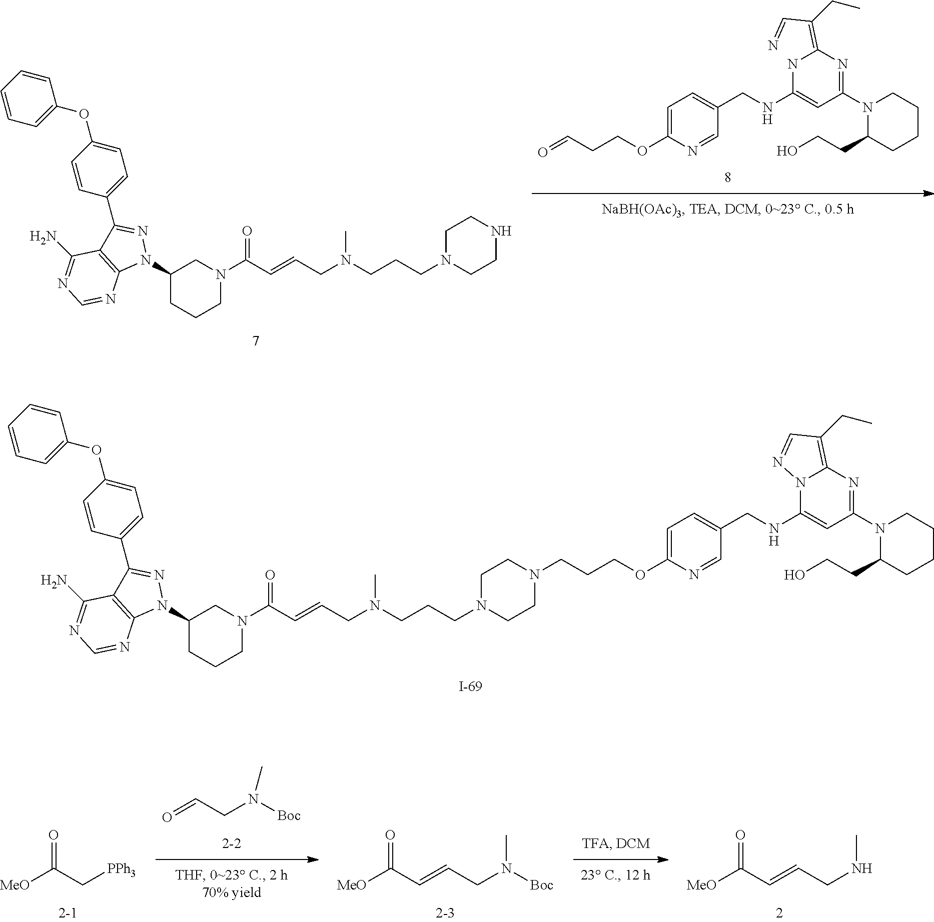

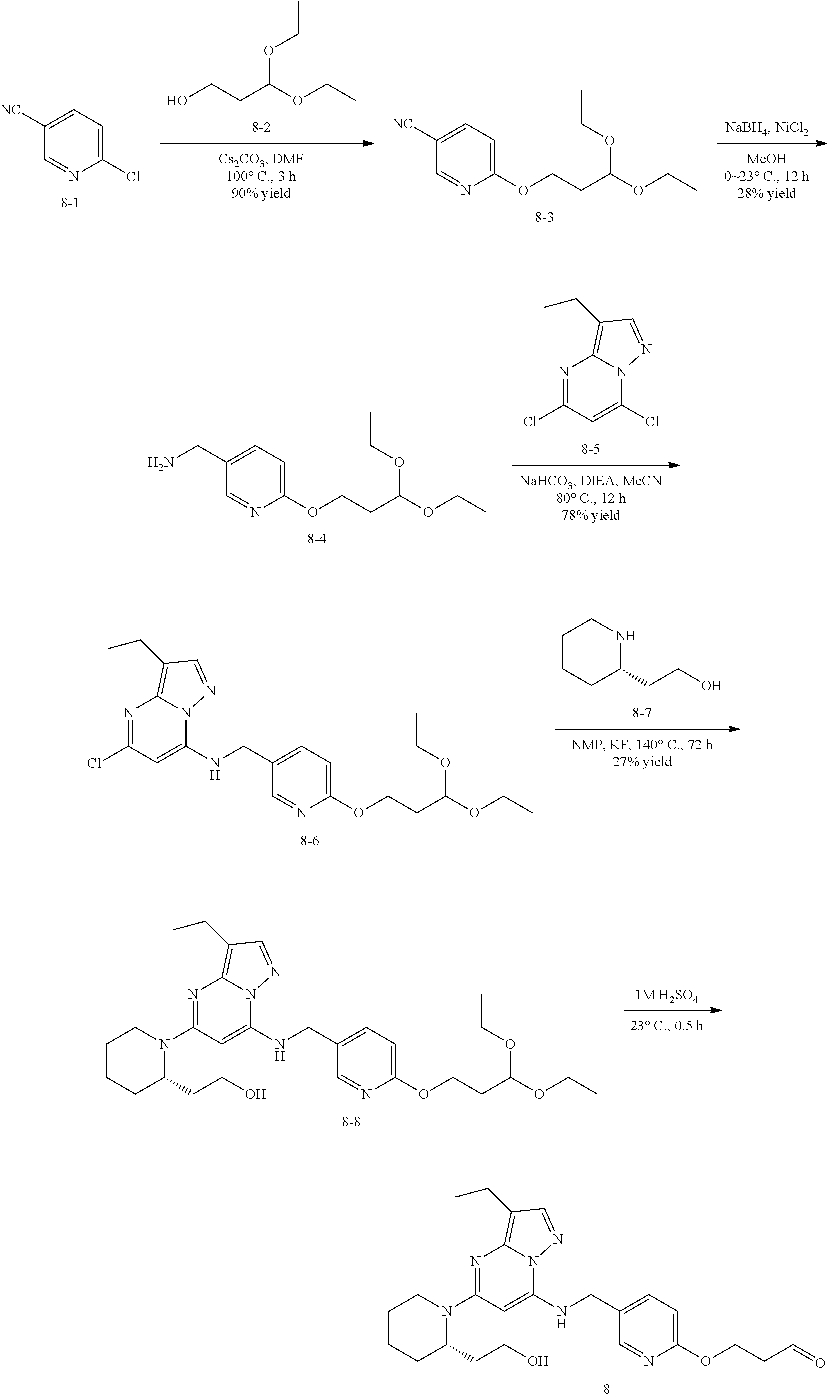

General Information: Synthetic route for compound 5 is described in WO2017/39425, 2017, A¹.

Step 1. To a solution of methyl 2-(triphenyl-2.5-phosphanylidene) acetate (19.3 g, 57.7 mmol, 1.0 equiv) in THF (130 mL) was added tert-butyl N-methyl-N-(2-oxoethyl) carbamate (10.0 g, 57.7 mmol, 1.0 equiv) at 0˜5° C. The mixture was stirred at 23° C. for 2 h. The reaction mixture was concentrated under reduced pressure to remove DCM and purified by flash silica gel chromatography (ISCOR; 120 g SepaFlash® Silica Flash Column, Eluent of 0˜10% Ethyl acetate/Petroleum ether gradient at 100 mL/min) to afford methyl (E)-4-[tert-butoxycarbonyl(methyl)amino]but-2-enoate (9.3 g, 40.6 mmol, 70% yield) as a colorless oil. ¹H NMR: (400 MHz, CDCl₃) δ 6.94-6.77 (m, 1H), 5.86 (d, J=15.6 Hz, 1H), 3.97-3.95 (m, 2H), 3.74 (s, 3H), 2.85 (s, 3H), 1.45 (s, 9H).

Step 2. To a solution of methyl (E)-4-[tert-butoxycarbonyl(methyl)amino]but-2-enoate (9.3 g, 40.6 mmol, 1.0 equiv) in DCM (50 mL) was added TFA (23.1 g, 202 mmol, 15 mL, 4.9 equiv). The mixture was stirred at 23° C. for 12 h. The reaction mixture was concentrated under reduced pressure to remove DCM to afford methyl (E)-4-(methylamino)but-2-enoate (14.0 g, crude, TFA) as a brown oil. ¹H NMR: (400 MHZ, CDCl₃) δ 7.02-6.75 (m, 1H), 6.19 (d, J=16.0 Hz, 1H), 3.85 (d, J=5.6 Hz, 2H), 3.78 (s, 3H), 2.80 (t, J=5.2 Hz, 3H).

Step 3. To a solution of tert-butyl 4-[3-(p-tolylsulfonyloxy) propyl]piperazine-1-carboxylate (1.5 g, 3.9 mmol, 1.0 equiv) and methyl (E)-4-(methylamino)but-2-enoate (946 mg, 3.9 mmol, 1.0 equiv, TFA) in DCM (20 mL) was added TEA (1.2 g, 11.6 mmol, 1.6 mL, 3.0 equiv). The mixture was stirred at 50° C. for 12 h. The reaction mixture was concentrated under reduced pressure to remove DCM and then was purified by prep-HPLC (column: YMC Triart C18 250*50 mm*7 μm; mobile phase: [water (NH₃H2O)-ACN]; B %: 30%-60%, 20 min) to afford tert-butyl 4-[3-[[(E)-4-methoxy-4-oxo-but-2-enyl]-methyl-amino]propyl]piperazine-1-carboxylate (700 mg, 2.0 mmol, 50% yield) as a yellow oil. ¹H NMR: (400 MHZ, CDCl₃) δ 7.00-6.91 (m, 1H), 5.98 (d, J=15.6 Hz, 1H), 3.74 (s, 3H), 3.44-3.41 (m, 4H), 3.15-3.12 (m, 2H), 2.40-2.35 (m, 8H), 2.23 (s, 3H), 1.70-1.62 (m, 2H), 1.46 (s, 9H).

Step 4. To a solution of tert-butyl 4-[3-[[(E)-4-methoxy-4-oxo-but-2-enyl]-methyl-amino]propyl]piperazine-1-carboxylate (100 mg, 281 μmol, 1.0 equiv) in THF (2 mL) was added LiOH (15 mg, 365 μmol, 844 μL, 1.3 equiv) in H₂O (0.5 mL). The mixture was stirred at 23° C. for 3 h. The reaction mixture was concentrated under reduced pressure to remove THF and H₂O to afford (E)-4-((3-(4-(tert-butoxycarbonyl) piperazin-1-yl) propyl)(methyl)amino)but-2-enoic acid (91 mg, crude) as a yellow oil.

Step 5. To a solution of (E)-4-[3-(4-tert-butoxycarbonylpiperazin-1-yl) propyl-methyl-amino]but-2-enoic acid (96.3 mg, 282 μmol, 1.0 equiv) in DMF (4 mL) was added a solution of 3-(4-phenoxyphenyl)-1-[(3R)-3-piperidyl]pyrazolo[3,4-d]pyrimidin-4-amine (141 mg, 282 μmol, 1.0 equiv, TFA) in DMF (4 mL) was added DIEA (182 mg, 1.41 mmol, 245 μL, 5.0 equiv) to adjust pH about 7˜8. And was added HATU (161 mg, 423 μmol, 1.5 equiv). The mixture was stirred at 23° C. for 0.5 h. The mixture was purified by prep-HPLC (column: Waters xbridge 150*25 mm 10 μm; mobile phase: [water (NH₄HCO₃)-ACN]; B %: 41%-71%, 8 min) to afford tert-butyl 4-[3-[[(E)-4-[(3R)-3-[4-amino-3-(4-phenoxyphenyl) pyrazolo[3,4-d]pyrimidin-1-yl]-1-piperidyl]-4-oxo-but-2-enyl]-methyl-amino]propyl]piperazine-1-carboxylate (158 mg, 218 μmol, 77% yield, 97% purity) as a white solid. ¹H NMR: (400 MHZ, CDCl₃) δ 8.37 (s, 1H), 7.67-7.63 (m, 2H), 7.43-7.37 (m, 2H), 7.22-7.14 (m, 3H), 7.09 (d, J=7.6 Hz, 2H), 6.91-6.78 (m, 1H), 6.55-6.38 (m, 1H), 5.66-5.43 (m, 2H), 4.87-4.86 (m, 1H), 4.67-4.55 (m, 1H), 4.28-3.96 (m, 1H), 3.84-3.75 (m, 1H), 3.44 (s, 4H), 3.24-3.12 (m, 2H), 2.42-2.37 (m, 8H), 2.24 (s, 2H), 2.00 (d, J=8.8 Hz, 6H), 1.79-1.66 (m, 3H), 1.46 (s, 9H).

Step 6. To a solution of tert-butyl 4-[3-[[(E)-4-[(3R)-3-[4-amino-3-(4-phenoxyphenyl) pyrazolo[3,4-d]pyrimidin-1-yl]-1-piperidyl]-4-oxo-but-2-enyl]-methyl-amino]propyl]piperazine-1-carboxylate (90 mg, 126 μmol, 1.0 equiv) in DCM (3 mL) was added TFA (1.5 g, 13.0 mmol, 1 mL). The mixture was stirred at 23° C. for 0.5 h. The reaction mixture was concentrated under reduced pressure to afford (E)-1-[(3R)-3-[4-amino-3-(4-phenoxyphenyl) pyrazolo[3,4-d]pyrimidin-1-yl]-1-piperidyl]-4-[methyl (3-piperazin-1-ylpropyl)amino]but-2-en-1-one (91.0 mg crude, TFA) as a yellow oil.

Step 7. To a solution of 6-chloropyridine-3-carbonitrile (4.0 g, 28.8 mmol, 1.0 equiv) in DMF (40 mL) was added Cs₂CO₃ (18.8 g, 57.7 mmol, 2.0 equiv) and 3,3-diethoxypropan-1-ol (4.3 g, 28.9 mmol, 1.0 equiv). The mixture was stirred at 100° C. for 3 h. The reaction mixture was filtered and diluted with H₂O 50 mL and extracted with EtOAc (50 mL×4). The combined organic layers were washed with brine (30 mL×2), dried over Na₂SO₄, filtered and concentrated under reduced pressure to give a residue. The residue was purified by column chromatography (SiO₂, Petroleum ether/Ethyl acetate=I/O˜10/1) to afford 6-(3,3-diethoxypropoxy) pyridine-3-carbonitrile (7.2 g, 26.1 mmol, 90% yield) as a light yellow oil. 1H NMR: (400 MHZ, CDCl₃) δ 8.48-8.42 (m, 1H), 7.74 (d, J=8.8, 2.4 Hz, 1H), 6.78 (d, J=8.8 Hz, 1H), 4.70 (t, J=5.6 Hz, 1H), 4.43 (t, J=6.4 Hz, 2H), 3.69-3.62 (m, 2H), 3.56-3.46 (m, 2H), 2.12-2.04 (m, 2H), 1.21-1.16 (m, 6H).

Step 8. To a solution of 6-(3,3-diethoxypropoxy) pyridine-3-carbonitrile (7.20 g, 28.8 mmol, 1.0 equiv) and NiCl₂ (748 mg, 5.8 mmol, 0.2 equiv) in MeOH (90 mL) was added NaBH₄ (2.7 g, 72.2 mmol, 2.5 equiv) at 0° C. The mixture was stirred at 23° C. for 12 h. The reaction mixture was quenched by addition NH₄Cl 50 mL, and then diluted with H₂O 200 mL and extracted with EtOAc (100 mL×3). The combined organic layers were washed with brine (100 mL×3), dried over Na₂SO₄, filtered and concentrated under reduced pressure to give a residue. The residue was purified by column chromatography (SiO₂, Petroleum ether/Ethyl acetate=1/1 ˜ 2/1˜0/1˜Ethyl acetate/MeOH=10/1) to afford [6-(3,3-diethoxypropoxy)-3-pyridyl]methanamine (2.1 g, 8.1 mmol, 28% yield) as a green oil. ¹H NMR: (400 MHZ, CDCl₃) δ 8.05 (s, 1H), 7.59 (d, J=7.2 Hz, 1H), 6.74 (d, J=6.4 Hz, 1H), 4.75 (t, J=5.6 Hz, 1H), 4.37-4.36 (m, 3H), 3.77-3.46 (m, 5H), 2.13-2.05 (m, 2H), 1.21 (t, J=7.2 Hz, 6H).

Step 9. To a solution of [6-(3,3-diethoxypropoxy)-3-pyridyl]methanamine (2.1 g, 8.3 mmol, 1.0 equiv) and 5,7-dichloro-3-ethyl-pyrazolo[1,5-a]pyrimidine (1.8 g, 8.3 mmol, 1.0 equiv) in MeCN(40 mL) was added DIEA (1.1 g, 8.3 mmol, 1.4 mL, 1.0 equiv) and NaHCO₃ (3.4 g, 41.2 mmol, 1.6 mL, 5.0 equiv). The mixture was stirred at 80° C. for 12 h. The reaction mixture was diluted with H₂O 50 mL and extracted with EtOAc (100 mL×2). The combined organic layers was washed with brine (50 mL×2), dried over Na₂SO₄, filtered and concentrated under reduced pressure to give a residue. The residue was purified by column chromatography (SiO₂, Petroleum ether/Ethyl acetate=10/1 to 20/1) to afford 5-chloro-N-[[6-(3,3-diethoxypropoxy)-3-pyridyl]methyl]-3-ethyl-pyrazolo[1,5-a]pyrimidin-7-amine (2.90 g, 6.51 mmol, 78% yield, 97% purity) as a yellow solid. ¹H NMR: (400 MHZ, CDCl₃) δ 8.17 (d, J=2.4 Hz, 1H), 7.85 (s, 1H), 7.58 (d, J=8.4, 2.4 Hz, 1H), 6.76 (d, J=8.4 Hz, 1H), 6.61 (t, J=5.6 Hz, 1H), 5.94 (s, 1H), 4.75 (t, J=5.6 Hz, 1H), 4.50 (d, J=5.6 Hz, 2H), 4.39 (t, J=12.8, 2H), 3.76-3.62 (m, 2H), 3.60-3.48 (m, 2H), 2.82-2.71 (m, 2H), 2.16-2.07 (m, 2H), 1.32-1.27 (m, 3H), 1.22 (t, J=7.2 Hz, 6H).

Step 10. To a solution of 5-chloro-N-[6-(3,3-diethoxypropoxy)-3-pyridyl]methyl]-3-ethyl-pyrazolo[1,5-a]pyrimidin-7-amine (2.9 g, 6.8 mmol, 1.0 equiv) and 2-[(2S)-2-piperidyl]ethanol (1.7 g, 13.3 mmol, 2.0 equiv) in NMP (7 mL) was added KF (3.1 g, 53.4 mmol, 1.3 mL, 8.0 equiv). The mixture was stirred at 140° C. for 72 h. The reaction mixture was diluted with H₂O 200 mL and extracted with EtOAc (50 mL×3). The combined organic layers were washed with brine (50 mL×3), dried over Na₂SO₄, filtered and concentrated under reduced pressure to give a residue. The residue was purified by column chromatography (SiO₂, Petroleum ether/Ethyl acetate=5/1 to 1/1) to afford 2-[(2S)-1-[7-[[6-(3,3-diethoxypropoxy)-3-pyridyl]methylamino]-3-ethyl-pyrazolo[1,5-a]pyrimidin-5-yl]-2-piperidyl]ethanol (1.0 g, 1.8 mmol, 27% yield) as a light yellow oil. ¹H NMR: (400 MHZ, CDCl₃) δ=8.17 (d, J=2.4 Hz, 1H), 7.69-7.54 (m, 2H), 6.75 (d, J=8.4 Hz, 1H), 6.37-6.19 (m, 1H), 5.30 (s, 1H), 4.76 (t, J=5.6 Hz, 1H), 4.44 (d, J=5.6 Hz, 2H), 4.39 (t, J=6.4 Hz, 2H), 4.17-4.08 (m, 1H), 3.78-3.64 (m, 3H), 3.63-3.49 (m, 3H), 3.41-3.27 (m, 1H), 3.10-2.99 (m, 1H), 2.66-2.54 (m, 2H), 2.14-2.07 (m, 2H), 1.87-1.44 (m, 9H), 1.28-1.25 (m, 3H), 1.22 (t, J=7.2 Hz, 6H).

Step 11. To a solution of H2SO₄ (1 M, 8 mL, 21.0 equiv) was added 2-[(2S)-1-[7-[[6-(3,3-diethoxypropoxy)-3-pyridyl]methylamino]-3-ethyl-pyrazolo[1,5-a]pyrimidin-5-yl]-2-piperidyl]ethanol (200 mg, 379 μmol, 1.0 equiv). The mixture was stirred at 23° C. for 0.5 h. The reaction mixture was quenched by addition NaHCO₃14 mL, and then diluted with H₂O 5 mL and extracted with DCM (5 mL×6). The combined organic layers were washed with brine (5 mL×2), dried over Na₂SO₄, filtered and concentrated under reduced pressure to give a crude product 3-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]propanal (170 mg, crude) as a pink oil.

Step 12. To a solution of (E)-1-[(3R)-3-[4-amino-3-(4-phenoxyphenyl) pyrazolo[3,4-d]pyrimidin-1-yl]-1-piperidyl]-4-[methyl (3-piperazin-1-ylpropyl)amino]but-2-en-1-one (91 mg, 126 μmol, 1.0 equiv, TFA) in DCM (2 mL) was added TEA (145 mg, 1.44 mmol, 0.2 mL, 11.0 equiv) to adjust pH about 7 ˜ 8 at 0° C. NaBH (OAc)3 (268 mg, 1.3 mmol, 10.0 equiv) and 3-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]propanal (57 mg, 126 μmol, 1.0 equiv) was added dropwise at 0° C. The mixture was stirred at 23° C. for 0.5 h. The reaction mixture was quenched by addition NaHCO₃15 mL, and then diluted with H₂O 5 mL and extracted with DCM (10 mL×3). The combined organic layers were washed with brine (5 mL×2), dried over Na₂SO₄, filtered and concentrated under reduced pressure to give a crude product. The crude product purified by prep-HPLC (column: Phenomenex C18 150*25 mm*10 μm; mobile phase: [water (NH₄HCO₃)-ACN]; B %: 50%-80%, 5 min) to afford (E)-1-[(3R)-3-[4-amino-3-(4-phenoxyphenyl) pyrazolo[3,4-d]pyrimidin-1-yl]-1-piperidyl]-4-[3-[4-[3-[5-[3-ethyl-5-[(2S)-2-(2-hydroxyethyl)-1-piperidyl]pyrazolo[1,5-a]pyrimidin-7-yl]amino]methyl]-2-pyridyl]oxy]propyl]piperazin-1-yl]propyl-methyl-amino]but-2-en-1-one (15 mg, 14 μmol, 11% yield, 97% purity) as a white solid. ¹H NMR: (400 MHZ, MeOD) δ=8.29-8.22 (m, 1H), 8.18 (s, 1H), 7.75 (d, J=8.4 Hz, 1H), 7.70-7.62 (m, 3H), 7.45-7.33 (m, 2H), 7.22-7.03 (m, 5H), 6.80-6.76 (m, 1H), 6.75-6.65 (m, 1H), 6.64-6.45 (m, 1H), 5.51 (s, 1H), 4.53 (s, 2H), 4.29 (t, J=5.6 Hz, 2H), 4.23-3.84 (m, 4H), 3.61-3.45 (m, 2H), 3.42-3.35 (m, 2H), 3.24 (d, J=4.4 Hz, 1H), 3.16-2.93 (m, 3H), 2.60-2.48 (m, 7H), 2.47-2.30 (m, 7H), 2.29-2.15 (m, 6H), 2.14-2.04 (m, 2H), 2.01-1.90 (m, 2H), 1.79-1.56 (m, 10H), 1.54-1.42 (m, 1H), 1.22 (t, J=7.2 Hz, 3H). LC-MS: MS (ES⁺): RT=2.256 min, m/z=524.0 [1/2M+H⁺], LCMS method 05.

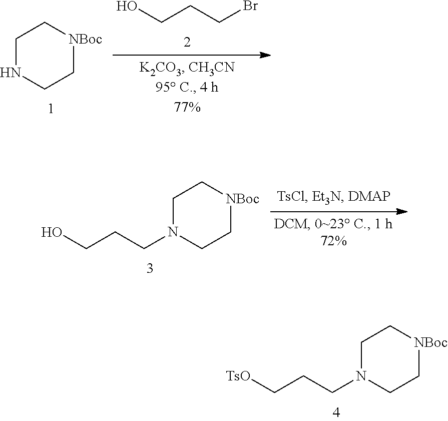 [SMILES: CC(C)(C)OC(=O)N1CCNCC1 ; OCCCBr ; CC(C)(C)OC(=O)N1CCN(CCCO)CC1 ; Cc1ccc(S(=O)(=O)OCCCN2CCN(C(=O)OC(C)(C)C)CC2)cc1]

Step 1. A mixture of tert-butyl piperazine-1-carboxylate (5.00 g, 26.8 mmol, 1.0 equiv), 3-bromopropan-1-ol (4.00 g, 29.2 mmol, 2.64 mL, 1.1 equiv), and K₂CO₃ (6.80 g, 49.6 mmol, 1.8 equiv) in CH₃CN(135 mL) was stirred at 95° C. for 4 h. The solvent was removed under reduced pressure and the residue was taken up in DCM (300 mL) and washed with H₂O (200 mL×2), brine (200 mL×2), dried over Na₂SO₄ and concentrated under reduced pressure. The residue was purified by column chromatography (SiO₂, DCM/MeOH=I/O ˜ 20/1) to afford tert-butyl 4-(3-hydroxypropyl) piperazine-1-carboxylate (5.00 g, 20.7 mmol, 77% yield) as a white solid. ¹H NMR: (400 MHZ, CDCl₃) δ 3.84-3.78 (m, 2H), 3.46-3.40 (m, 4H), 2.65-2.58 (m, 2H), 2.50-2.42 (m, 4H), 1.78-1.69 (m, 2H), 1.46 (s, 9H).

Step 2. To a solution of tert-butyl 4-(3-hydroxypropyl) piperazine-1-carboxylate (5.00 g, 20.4 mmol, 1.0 equiv), N,N-dimethylpyridin-4-amine (250 mg, 2.05 mmol, 0.1 equiv) and Et₃N(4.10 g, 40.9 mmol, 5.70 mL, 2.0 equiv) in DCM (50 mL) was added 4-methyl benzenesulfonyl chloride (3.90 g, 20.4 mmol, 1.0 equiv) at 0° C. The mixture was stirred at 23° C. for 1 h. The reaction mixture was concentrated under reduced pressure to remove DCM to afford crude product. The crude product was purified by column chromatography (SiO₂, DCM/MeOH=I/O to 50/1) to afford tert-butyl 4-[3-(p-tolylsulfonyloxy) propyl]piperazine-1-carboxylate (6.35 g, 14.8 mmol, 72% yield, 93% purity) as a yellow oil. ¹H NMR: (400 MHZ, CDCl₃) δ 7.72-7.70 (m, 2H), 7.35-7.15 (m, 2H), 4.04-4.03 (m, 2H), 3.39-3.20 (m, 4H), 2.42-2.15 (m, 9H), 1.73-1.67 (m, 2H), 1.38 (s, 9H).

### Example 49—the Synthetic Route for I-70

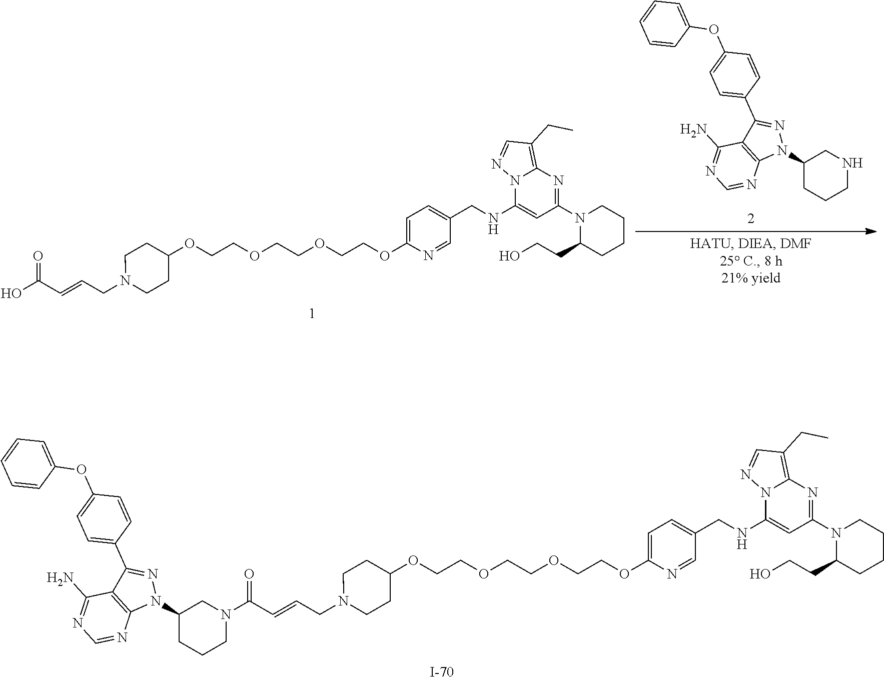

General Information: Synthetic route for compound 2 is described in WO2017/39425.

To a solution of (R)-3-(4-phenoxyphenyl)-1-(piperidin-3-yl)-1H-pyrazolo[3,4-d]pyrimidin-4-amine (21 mg, 54 μmol, 1.0 equiv) in DMF (2 mL) was added HATU (31 mg, 82 μmol, 1.5 equiv) and DIEA (21 mg, 163 μmol, 28 μL, 3.0 equiv), (S,E)-4-(4-(2-(2-(2-((5-(((3-ethyl-5-(2-(2-hydroxyethyl) piperidin-1-yl) pyrazolo[1,5-a]pyrimidin-7-yl)amino) methyl) pyridin-2-yl)oxy)ethoxy)ethoxy)ethoxy) piperidin-1-yl) but-2-enoic acid (38 mg, 54 μmol, 1.0 equiv). The mixture was stirred at 25° C. for 8 h. The reaction mixture was concentrated under reduced pressure to remove solvent. The residue was diluted with water 30 mL and extracted with EA mL (30 mL×3). The combined organic layers were washed with water (25 mL×2), dried over Na₂SO₄ filtered and concentrated under reduced pressure to give a residue. The crude product was purified by reversed-phase HPLC column: Waters xbridge 150*25 mm*10 μm; mobile phase: [water (NH₄HCO₃)-ACN]; B %: 40%-70%, 8 min. Compound (E)-1-((R)-3-(4-amino-3-(4-phenoxyphenyl)-1H-pyrazolo[3,4-d]pyrimidin-1-yl) piperidin-1-yl)-4-(4-(2-(2-(2-((5-(((3-ethyl-5-((S)-2-(2-hydroxyethyl) piperidin-1-yl) pyrazolo[1,5-a]pyrimidin-7-yl)amino) methyl) pyridin-2-yl)oxy)ethoxy)ethoxy)ethoxy) piperidin-1-yl) but-2-en-1-one (12 mg, 11 μmol, 21% yield) was obtained as a white solid. ¹H NMR: (400 MHZ, CD₃OD) δ=8.16-8.13 (d, J=10.0 Hz, 1H), 8.09-8.06 (d, J=12.4 Hz, 1H), 7.67 (m, 4H), 7.31-7.30 (t, J=7.2 Hz, 1H), 7.34-7.24 (m, 1H), 7.03 (m, 2H), 7.00-6.97 (m, 1H), 6.72-6.64 (m, 1H), 6.56-6.48 (m, 1H), 6.32-6.20 (m, 1H), 5.41 (s, 1H), 4.44-4.40 (m, 2H), 4.34-4.27 (m, 2H), 4.03-3.83 (m, 3H), 3.76-3.69 (m, 2H), 3.36-3.24 (m, 1H), 3.60-3.23 (m, 13H), 3.01 (s, 1H), 2.81 (s, 2H), 2.65-2.57 (m, 1H), 2.49-2.41 (m, 3H), 32.31-2.18 (m, 1H), 2.15-2.06 (m, 2H), 2.00-1.94 (m, 2H), 1.80-1.73 (m, 1H), 1.71-1.46 (m, 10H), 1.43-1.34 (m, 2H), 1.31-1.17 (m, 1H), 1.17-1.06 (m, 5H). LC-MS: MS (ES⁺): RT=2.473 min, m/z=1064.4 [M+H⁺], LCMS Method 05.

### Example 50—the Synthetic Route for I-71

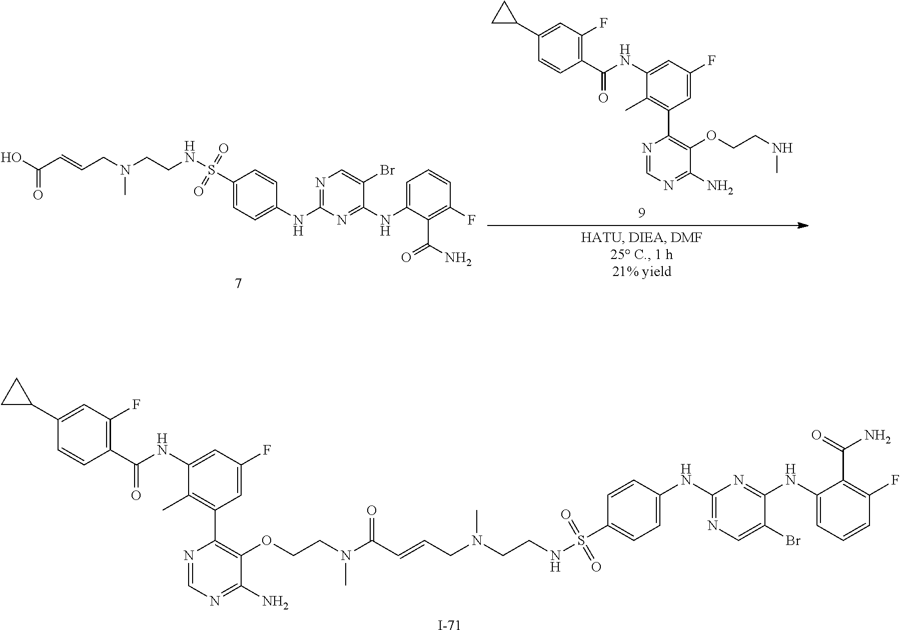

General Information: Synthetic route for compound 9 is described in US2015/152068.

Step 1. A mixture of (E)-4-[2-[[4-[[5-bromo-4-(2-carbamoyl-3-fluoro-anilino) pyrimidin-2-yl]amino]phenyl]sulfonylamino]ethyl-methyl-amino]but-2-enoic acid (80 mg, 129 μmol, 1.0 equiv), N-[3-[6-amino-5-[2-(methylamino)ethoxy]pyrimidin-4-yl]-5-fluoro-2-methyl-phenyl]-4-cyclopropyl-2-fluoro-benzamide (58 mg, 129 μmol, 1.0 equiv) and DIEA (50 mg, 386 μmol, 67μ, 3.0 equiv) in DMF (2.0 mL) was added HATU (64 mg, 167 μmol, 1.3 equiv), then the mixture was degassed and purged with N₂ for 3 times, and then the mixture was stirred at 25° C. for 1 h under N₂ atmosphere. Filter to remove precipitated impurities. The crude product was purified by reversed-phase HPLC (column: Phenomenex C18 150×25 mm×10 μm; mobile phase: [water (NH₄HCO₃)-ACN]; B %: 42%-72%,8 min) to afford (E)-N-(3-(6-amino-5-(2-(4-((2-(4-((5-bromo-4-((2-carbamoyl-3-fluorophenyl)amino)pyrimidin-2-yl)amino)phenylsulfonamido) ethyl)(methyl)amino)-N-methylbut-2-enamido)ethoxy) pyrimidin-4-yl)-5-fluoro-2-methylphenyl)-4-cyclopropyl-2-fluorobenzamide (28 mg, 27 μmol, 21% yield) as off-white solid.¹H NMR: (400 MHZ, DMSO-d6) δ=10.15 (d, J=4.0 Hz, 1H), 9.94 (d, J=10.0 Hz, 1H), 9.83-9.53 (m, 1H), 8.43-8.32 (m, 1H), 8.29-8.05 (m, 4H), 7.85 (d, J=7.6 Hz, 2H), 7.72-7.46 (m, 5H), 7.14-6.87 (m, 6H), 4.21 (s, 1H), 3.64-3.43 (m, 5H), 3.17-2.87 (m, 3H), 2.68 (s, 3H), 2.02-1.95 (m, 6H), 1.74-1.63 (m, 2H), 1.06-1.01 (m, 2H), 0.81-0.73 (m, 2H). LC-MS: MS (ES⁺): RT=2.468 min, m/z=530 [1/2M+H⁺], LCMS method 05.

### Example 51—the Synthetic Route for I-72

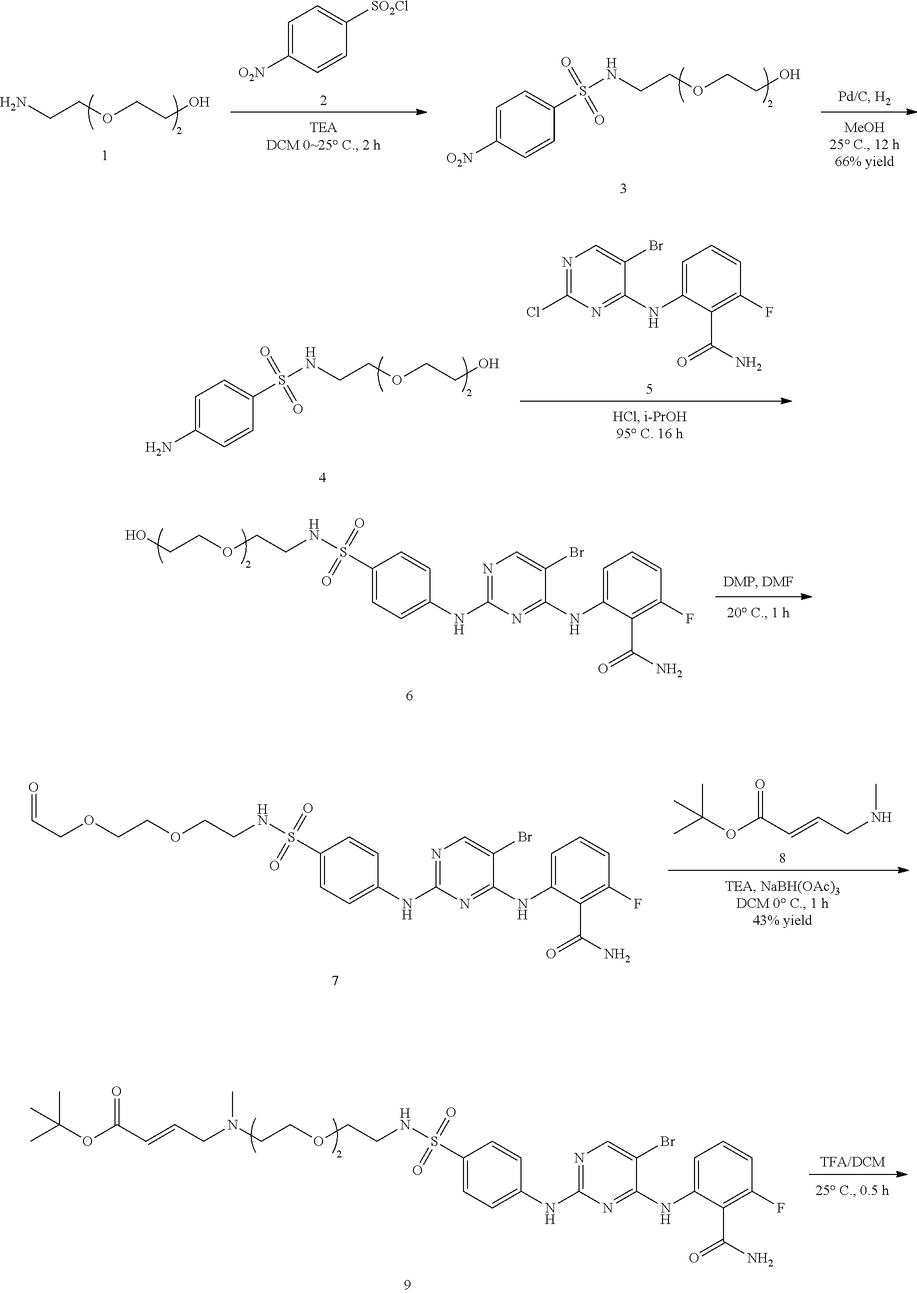

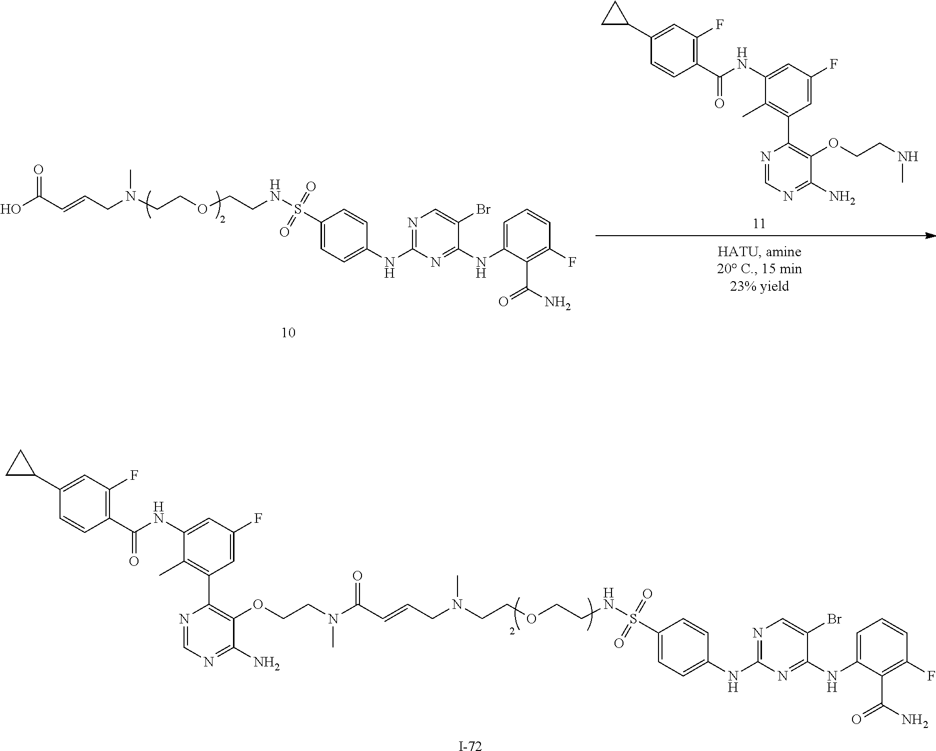

General Information: Synthetic route for compound 5 is described in Angewandte Chemie-International Edition (2020) vol. 59, 13865. Synthetic route for compound 11 is provided in US2015/152068.

Step 1. To a solution of 2-[2-(2-aminoethoxy)ethoxy]ethanol (15.0 g, 101 mmol, 1.0 equiv), TEA (30.5 g, 301 mmol, 41.9 mL, 3.0 equiv) in DCM (150 mL) was added a solution of 4-nitrobenzenesulfonyl chloride (22.3 g, 101 mmol, 1.0 equiv) in DCM (50 mL) at 0° C. under N₂. The mixture was stirred at 25° C. for 2 h. The reaction mixture was quenched by addition 0.1M HCl 200 mL, and then diluted 10% NaOH until the pH-7˜8 and extracted with DCM (200 mL×3). Dried over Na₂SO₄, filtered and concentrated under reduced pressure to afford N-[2-[2-(2-hydroxyethoxy)ethoxy]ethyl]-4-nitro-benzenesulfonamide (37.3 g, crude) as yellow solid. ¹H NMR: (400 MHZ, CD₃OD) δ 8.43 (d, J=6.8 Hz 2H), 8.18 (d, J=8.8 Hz, 2H), 3.71-3.62 (m, 2H), 3.60-3.39 (m, 8H), 3.22-3.08 (m, 2H).

Step 2. To a solution of N-[2-[2-(2-hydroxyethoxy)ethoxy]ethyl]-4-nitro-benzenesulfonamide (10.0 g, 29.9 mmol, 1.0 equiv) in MeOH (100 mL) was added Pd/C (3 g, 10% purity) and H₂. The mixture was stirred at 25° C. for 12 h. The reaction mixture was filtered and concentrated under reduced pressure to give a residue. The residue was purified by prep-HPLC (column: YMC Triart C18 250*50 mm*7 μm; mobile phase: [water (NH₄HCO₃)-ACN]; B %: 2%-32%,20 min) to afford 4-amino-N-[2-[2-(2-hydroxyethoxy)ethoxy]ethyl]benzenesulfonamide ¹H NMR: (400 MHZ, DMSO-d₆) δ 7.41 (d, J=8.4 Hz, 2H), 7.19-7.12 (m, 1H), 6.61 (d, J=8.4 Hz, 2H), 3.49-3.45 (m, 4H), 3.44-3.41 (m, 2H), 3.40-3.33 (m, 4H), 3.16 (s, 1H), 2.79 (t, J=6.0 Hz, 2H).

Step 3. To a solution of 4-amino-N-[2-[2-(2-hydroxyethoxy)ethoxy]ethyl]benzenesulfonamide (1.0 g, 3.3 mmol, 1.0 equiv) in i-PrOH (30 mL) was added 2-[(5-bromo-2-chloro-pyrimidin-4-yl)amino]-6-fluoro-benzamide (568 mg, 1.6 mmol, 0.5 equiv) and HCl (12 M, 273 μL, 1.0 equiv). The mixture was stirred at 95° C. for 16 h. The reaction mixture concentrated under reduced pressure to afford 2-[[5-bromo-2-[4-[2-[2-(2-hydroxyethoxy) ethoxy]ethylsulfamoyl]anilino]pyrimidin-4-yl]amino]-6-fluoro-benzamide (0.9 g,crude) as yellow solid. ¹H NMR: (400 MHZ, DMSO-d₆) δ 10.21 (s, 1H), 8.41 (s, 1H), 8.20-8.14 (m, 2H), 7.81 (d, J=8.8 Hz, 2H), 7.65 (d, J=8.8 Hz, 2H), 7.52 (t, J=6.8, 8.2 Hz, 2H), 7.11 (t, J=9.2 Hz, 1H), 3.48-3.43 (m, 6H), 3.39-3.36 (m, 4H), 2.86 (t, J=5.6 Hz, 2H).

Step 4. A mixture of 2-[[5-bromo-2-[4-[2-[2-(2-hydroxyethoxy)ethoxy]ethylsulfamoyl]anilino]pyrimidin-4-yl]amino]-6-fluoro-benzamide (150 mg, 244 μmol, 1.0 equiv), DMP (311 mg, 733 μmol, 227 μL, 3.0 equiv), in DMF (0.3 mL) was stirred at 20° C. for 1 h. The mixture was filtration. The mixture was use to next step directly.

Step 5. A mixture of 2-[5-bromo-2-[4-[2-[2-(2-oxoethoxy)ethoxy]ethylsulfamoyl]anilino]pyrimidin-4-yl]amino]-6-fluoro-benzamide (150 mg, 245 μmol, 1.0 equiv) and tert-butyl(E)-4-(methylamino)but-2-enoate (126 mg, 736 μmol, 3.0 equiv) in DCM (2 mL) was add NaBH (OAc)3 (780 mg, 3.7 mmol, 15.0 equiv) and TEA (372 mg, 3.7 mmol, 512 μL, 15.0 equiv). The mixture was stirred at 0° C. for 1 h. The reaction mixture was quenched by addition sat.NaHCO₃ (2 mL), and then extracted with DCM (2 mL×3). Dried over Na₂SO₄, filtered and concentrated under reduced pressure to give a residue. The residue was purified by prep-HPLC (column: Phenomenex C18 150*25 mm*10 μm; mobile phase: [water (NH₄HCO₃)-ACN]; B %: 38%-68%,8 min) to afford tert-butyl(E)-4-[2-[2-[2-[[4-[[5-bromo-4-(2-carbamoyl-3-fluoro-anilino) pyrimidin-2-yl]amino]phenyl]sulfonylamino]ethoxy]ethoxy]ethyl-methyl-amino]but-2-enoate (80 mg, 104 μmol, 43% yield) as white solid. ¹HNMR: (400 MHZ, CD₃OD) δ 8.35 (d, J=8.4 Hz, 1H), 8.27 (s, 1H), 7.85-7.81 (m, 2H), 7.73-7.69 (m, 2H), 7.71-7.70 (m, 1H), 7.01-6.99 (m, 1H), 6.96-6.78 (m, 1H), 5.90 (t, J=15.6 Hz, 1H), 3.60-3.44 (m, 12H), 3.18 (dd, J=6.4 Hz, 2H), 2.25 (s, 3H), 1.46 (s, 9H).

Step 6. A mixture of tert-butyl(E)-4-[2-[2-[2-[[4-[[5-bromo-4-(2-carbamoyl-3-fluoro-anilino) pyrimidin-2-yl]amino]phenyl]sulfonylamino]ethoxy]ethoxy]ethyl-methyl-amino]but-2-enoate (36 mg, 47 μmol, 0.05 equiv), TFA (106 mg, 935 μmol, 69 μL, 1.0 equiv) in DCM (2 mL) was degassed and purged with N₂ for 3 times, and then the mixture was stirred at 25° C. for 0.5 h under N₂ atmosphere. The reaction mixture was concentrated under reduced pressure to give a residue. The mixture was use to next step directly.

Step 7. To a solution of (E)-4-[2-[2-[2-[[4-[[5-bromo-4-(2-carbamoyl-3-fluoro-anilino) pyrimidin-2-yl]amino]phenyl]sulfonylamino]ethoxy]ethoxy]ethyl-methyl-amino]but-2-enoic acid (50 mg, 70 μmol, 1.0 equiv) in DMF (2 mL) and N-[3-[6-amino-5-[2-(methylamino)ethoxy]pyrimidin-4-yl]-5-fluoro-2-methyl-phenyl]-4-cyclopropyl-2-fluoro-benzamide (32 mg, 56 μmol, 0.8 equiv, TFA) was added DIEA (9 mg, 70 μmol, 12 μL, 1.0 equiv) and HATU (27 mg, 70 μmol, 1.0 equiv). The mixture was stirred at 20° C. for 15 min. The reaction mixture was concentrated under reduced pressure to give a residue. The residue was purified by prep-HPLC (column: Phenomenex C18 150*25 mm*10 μm; mobile phase: [water (NH₄HCO₃)-ACN]; B %: 35%-65%, 8 min) to afford 2-[[2-[4-[2-[2-[2-[[(E)-4-[2-[4-amino-6-[3-[(4-cyclopropyl-2-fluoro-benzoyl)amino]-5-fluoro-2-methyl-phenyl]pyrimidin-5-yl]oxyethyl-methyl-amino]-4-oxo-but-2-enyl]-methyl-amino]ethoxy]ethoxy]ethylsulfamoyl]anilino]-5-bromo-pyrimidin-4-yl]amino]-6-fluoro-benzamide (20 mg, 16 μmol, 23% yield, 89% purity) as white solid. ¹H NMR: (400 MHZ, CD₃OD) δ 8.35 (d, J=8.8 Hz, 1H), 8.26 (s, 1H), 8.20 (d, J=11.6 Hz, 1H), 7.80-7.83 (m, 3H), 7.73-7.66 (m, 3H), 7.49 (d, J=6.4 Hz, 1H), 7.07-6.95 (m, 4H), 6.84-6.58 (m, 2H), 3.73-3.66 (m, 2H), 3.56-3.41 (m, 12H), 2.87 (s, 2H), 2.59-2.53 (m, 4H), 2.25 (d, J=8.8 Hz, 4H), 2.13-1.99 (m, 5H), 1.12-1.06 (m, 2H), 0.82-0.77 (m, 2H). LC-MS: MS (ES⁺): RT=13.494 min, m/z=574.2 [1/2M+H⁺]; LCMS method 10.

### Example 52—the Synthetic Route for I-73

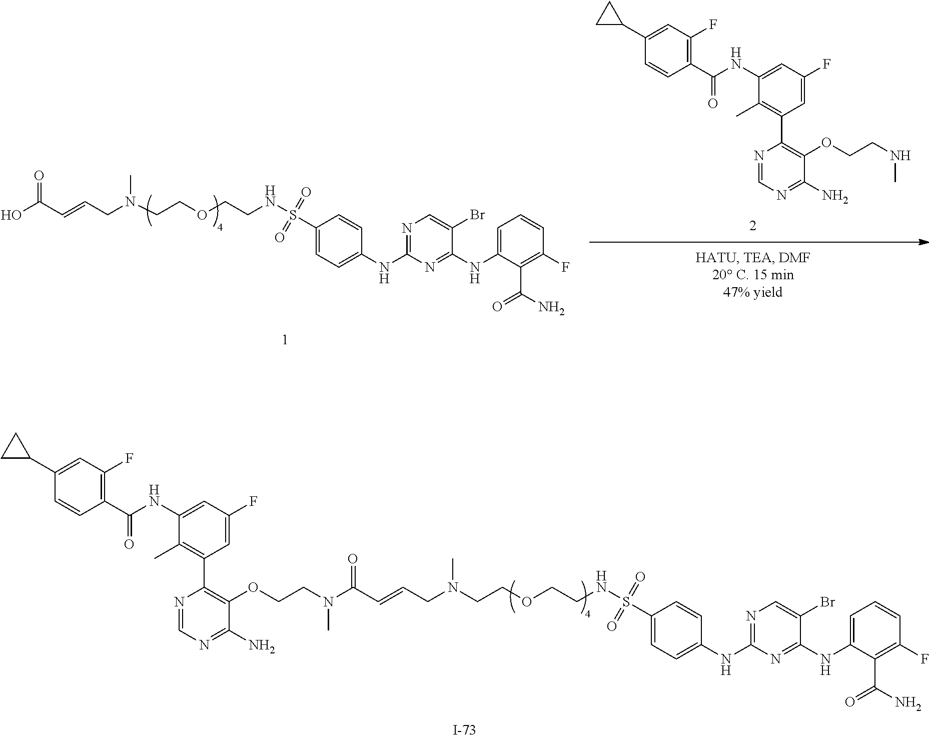

General Information: Synthetic route for compound 2 is described in US2015/152068.

Step 1. To a solution of (E)-4-[2-[2-[2-[2-[2-[[4-[[5-bromo-4-(2-carbamoyl-3-fluoro-anilino) pyrimidin-2-yl]amino]phenyl]sulfonylamino]ethoxy]ethoxy]ethoxy]ethoxy]ethyl-methyl-amino]but-2-enoic acid (40 mg, 50 μmol, 1.0 equiv) and N-[3-[6-amino-5-[2-(methylamino)ethoxy]pyrimidin-4-yl]-5-fluoro-2-methyl-phenyl]-4-cyclopropyl-2-fluoro-benzamide (23 mg, 40 μmol, 1.1 equiv, TFA) in DMF (2 mL) was added DIEA (32 mg, 250 μmol, 43 μL, 5.0 equiv) and HATU (19 mg, 50 μmol, 1.0 equiv). The mixture was stirred at 20° C. for 15 min. The reaction mixture was concentrated under reduced pressure to give a residue. The residue was purified by prep-HPLC (column: Phenomenex luna C18 150*25 mm*10 μm; mobile phase: [water (FA)-ACN]; B %: 22%-52%,8 min) to afford 2-[[2-[4-[2-[2-[2-[2-[2-[[(E)-4-[2-[4-amino-6-[3-[(4-cyclopropyl-2-fluoro-benzoyl)amino]-5-fluoro-2-methyl-phenyl]pyrimidin-5-yl]oxyethyl-methyl-amino]-4-oxo-but-2-enyl]-methyl-amino]ethoxy]ethoxy]ethoxy]ethoxy]ethylsulfamoyl]anilino]-5-bromo-pyrimidin-4-yl]amino]-6-fluoro-benzamide (30 mg, 24 μmol, 47% yield, 98% purity) as white solid. ¹H NMR: (400 MHz, CD₃OD) δ 8.36 (d, J=8.4 Hz, 1H), 8.26 (s, 1H), 8.22-8.16 (m, 1H), 7.86-7.75 (m, 3H), 7.73-7.63 (m, 3H), 7.53-7.44 (m, 1H), 7.08-6.92 (m, 4H), 6.76-6.54 (m, 2H), 3.73-3.67 (m, 2H), 3.63-3.42 (m, 20H), 3.04-3.00 (m, 2H), 2.90-2.77 (m, 4H), 2.60-2.43 (m, 4H), 2.11-1.99 (m, 4H), 1.09 (d, J=7.6 Hz, 2H), 0.79 (s, 2H). LC-MS: MS (ES⁺): RT=13.976 min, m/z=618.0 [M/2+H⁺]; LCMS method 05.

### Example 53—the Synthetic Route for I-74

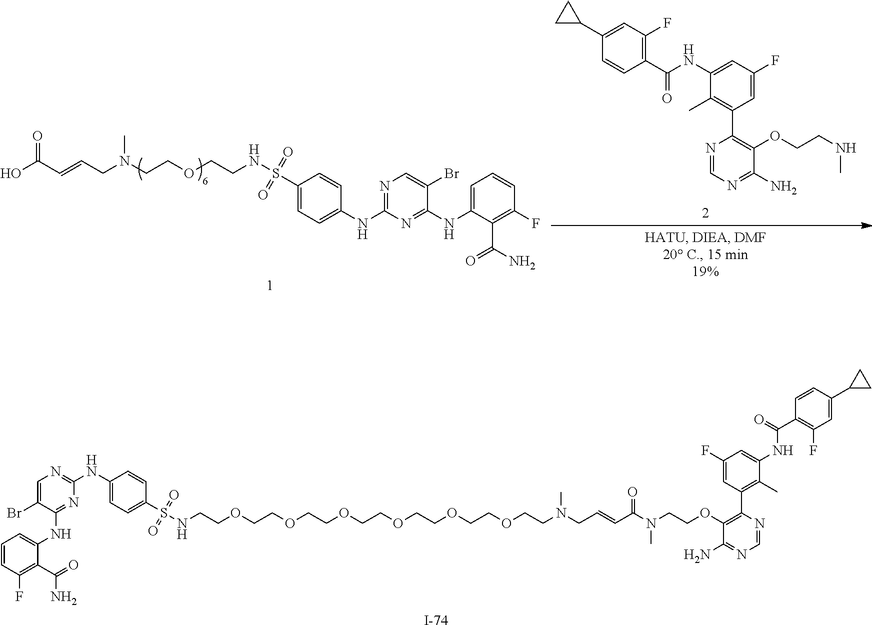

General Information: Synthetic route for compound 2 is described in US2015/152068.

Step 1. To a solution of (E)-4-[2-[2-[2-[2-[2-[2-[2-[[4-[[5-bromo-4-(2-carbamoyl-3-fluoro-anilino) pyrimidin-2-yl]amino]phenyl]sulfonylamino]ethoxy]ethoxy]ethoxy]ethoxy]ethoxy]ethoxy]ethyl-methyl-amino]but-2-enoic acid (42 mg, 42 μmol, 1.0 equiv, TFA) in DMF (1.5 mL) was added N-[3-[6-amino-5-[2-(methylamino)ethoxy]pyrimidin-4-yl]-5-fluoro-2-methyl-phenyl]-4-cyclopropyl-2-fluoro-benzamide (19 mg, 33 μmol, 0.8 equiv, TFA), DIEA (27 mg, 209 μmol, 36 μL, 5.0 equiv) and HATU (32 mg, 84 μmol, 2.0 equiv). The mixture was stirred at 20° C. for 0.2 h. The residue was purified by prep-HPLC (column: Welch Ultimate C18 150*25 mm*5 μm; mobile phase: [water (FA)-ACN]; B %: 25%-55%, 10.5 min) to afford 2-[[2-[4-[2-[2-[2-[2-[2-[2-[2-[[(E)-4-[2-[4-amino-6-[3-[(4-cyclopropyl-2-fluoro- benzoyl)amino]-5-fluoro-2-methylphenyl]pyrimidin5yl]oxyethylmethylamino]-4-oxobut-2-enyl]methylamino]ethoxy]ethoxy]ethoxy]ethoxy]ethoxy]ethoxy]ethylsulfamoyl]anilino]-5-bromo-pyrimidin-4-yl]amino]-6-fluoro-benzamide (11 mg, 8 μmol, 19% yield, 96% purity) as white solid.¹H NMR: (400 MHZ, CD₃OD) δ 8.36 (d, J=8.8 Hz, 1H), 8.27 (s, 1H), 8.20 (d, J=12.4 Hz, 1H), 7.83 (d, J=8.8 Hz, 3H), 7.71 (d, J=8.8 Hz, 3H), 7.57-7.45 (m, 1H), 7.06 (d, J=7.6 Hz, 1H), 7.03-6.97 (m, 3H), 6.81-6.52 (m, 2H), 3.72-3.42 (m, 30H), 3.04 (t, J=5.6 Hz, 2H), 2.90 (s, 4H), 2.66-2.44 (m, 5H), 2.14-1.95 (m, 4H), 1.14-1.04 (m, 2H), 0.80 (d, J=4.8 Hz, 2H). LC-MS: MS (ES⁺): RT=2.510 min, m/z=662.1 [M/2+H⁺]; LCMS method 05.

### Example 54—the Synthetic Route for I-75

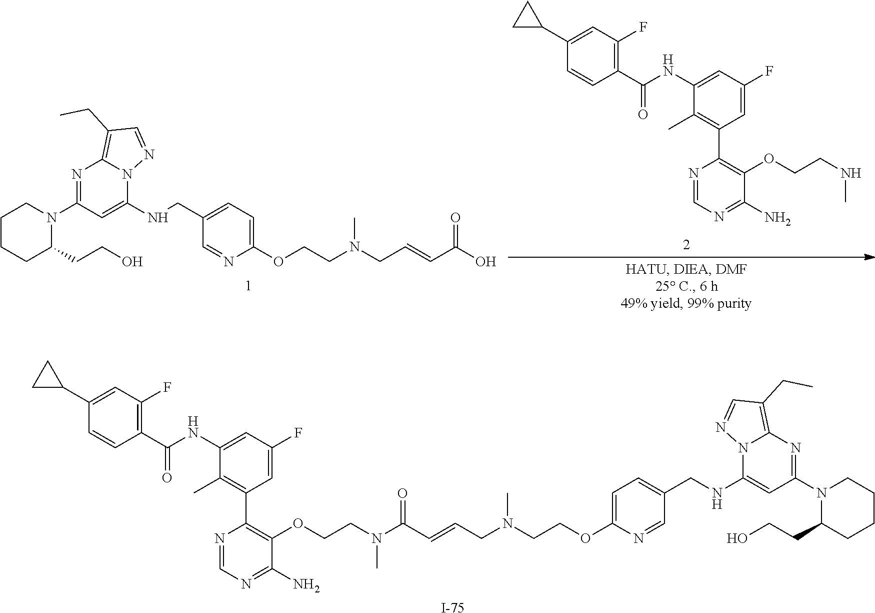

General Information: Synthetic route for compound 2 is described in US2015/152068, 2015, A¹.

Step 1. To a solution of (E)-4-[2-[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethyl-methyl-amino]but-2-enoic acid (50 mg, 93 μmol, 1.0 equiv) in DMF (5 mL) was added HATU (42 mg, 112 μmol, 1.2 equiv) and DIEA (36 mg, 279 μmol, 49 μL, 3.0 equiv). The mixture was added N-[3-[6-amino-5-[2-(methylamino)ethoxy]pyrimidin-4-yl]-5-fluoro-2-methyl-phenyl]-4-cyclopropyl-2-fluoro-benzamide (42 mg, 93 μmol, 1.0 equiv). The mixture was stirred at 25° C. for 6 h. The reaction mixture was concentrated to get the residue. The residue was purified by the prep-HPLC (column: Phenomenex C18 150*25 mm*10 μm; mobile phase: [water (NH₄HCO₃)-ACN]; B %: 44%-74%,5 min) to give the N-[3-[6-amino-5-[2-[[(E)-4-[2-[[5-[3-ethyl-5-[(2S)-2-(2-hydroxyethyl)-1-piperidyl]pyrazolo[1,5-a]pyrimidin-7-yl]amino]methyl]-2-pyridyl]oxy]ethyl-methyl-amino]but-2-enoyl]-methyl-amino]ethoxy]pyrimidin-4-yl]-5-fluoro-2-methyl-phenyl]-4-cyclopropyl-2-fluoro-benzamide (45 mg, 45 μmol, 49% yield, 99% purity) as a white solid. ¹H NMR: (400 MHZ, MeOH) δ=8. 8.13-8.26 (m, 2H), 7.62-7.90 (m, 4H), 6.91-7.12 (m, 3H), 6.70-6.88 (m, 2H), 6.65 (d, J=15.2 Hz, 1H), 6.47 (d, J=15.2 Hz, 1H), 5.54 (s, 1H), 4.48-4.64 (m, 4H), 4.32-4.45 (m, 2H), 3.96-4.13 (m, 1H), 3.50-3.81 (m, 6H), 3.37-3.43 (m, 2H), 3.26 (d, J=6.0 Hz, 1H), 2.94-3.08 (m, 1H), 2.86 (s, 1H), 2.78-2.84 (m, 2H), 2.53-2.60 (m, 3H), 2.36 (d, J=14.0 Hz, 3H), 1.90-2.20 (m, 4H), 1.62-1.77 (m, 6H), 1.42-1.58 (m, 1H), 1.19-1.29 (m, 3H), 1.03-1.11 (m, 2H), 0.75-0.83 (m, 2H) LC-MS: MS (ES⁺): RT=2.246 min, m/z=973.8 [M+H⁺]; LCMS method 05.

### Example 55—the Synthetic Route for I-76

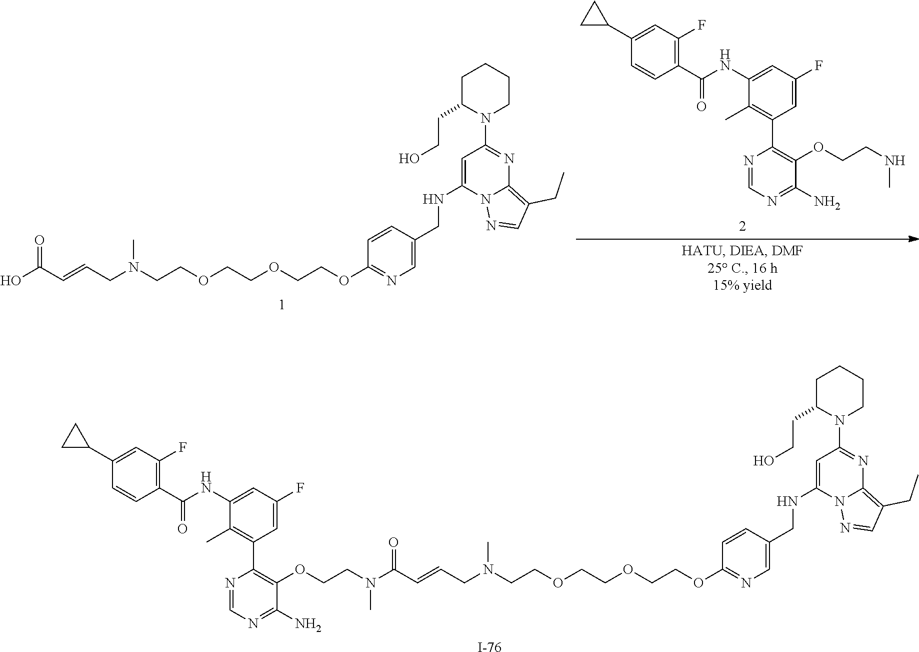

General Information: Synthetic route for compound 2 is described in US2015/152068, 2015, A¹.

Step 1. To a solution of N-(3-(6-amino-5-(2-(methylamino)ethoxy) pyrimidin-4-yl)-5-fluoro-2-methylphenyl)-4-cyclopropyl-2-fluorobenzamide (14 mg, 31 μmol, 1.0 equiv) in DMF (1 mL) was added HATU (18 mg, 48 μmol, 1.5 equiv) and DIEA (13 mg, 96 μmol, 16 μL, 3.0 equiv), (S,E)-4-((2-(2-(2-(5-(((3-ethyl-5-(2-(2-hydroxyethyl) piperidin-1-yl) pyrazolo[1,5-a]pyrimidin-7-yl)amino) methyl) pyridin-2-yl)ethoxy)ethoxy) ethyl)(methyl)amino)but-2-enoic acid (20 mg, 32 μmol, 1.0 equiv). The mixture was stirred at 25° C. for 16 h to give a yellow solution. The reaction mixture was diluted with H₂O (30 mL) and extracted with EA (20 mL×3). The combined organic layers were washed with water (25 mL×2), dried over Na₂SO₄, filtered and concentrated under reduced pressure to give a residue. The crude product was purified by reversed-phase HPLC column: Phenomenex C18 150*25 mm*10 μm; mobile phase: [water (NH₄HCO₃)-ACN]; B %: 42%-72%, 8 min. Compound (S,E)-N-(3-(6-amino-5-((1-(5-(((3-ethyl-5-(2-(2-hydroxyethyl) piperidin-1-yl) pyrazolo[1,5-a]pyrimidin-7-yl)amino) methyl) pyridin-2-yl)-9,14-dimethyl-13-oxo-3,6-dioxa-9,14-diazahexadec-11-en-16-yl)oxy) pyrimidin-4-yl)-5-fluoro-2-methylphenyl)-4-cyclopropyl-2-fluorobenzamide (5 mg, 5 μmol, 15% yield) was obtained as a yellow solid. ¹H NMR: (400 MHZ, CD₃OD) δ=8.15-8.05 (m, 2H), 7.69-7.61 (m, 3H), 7.56-7.50 (m, 1H), 6.96-6.91 (m, 1H), 6.83 (s, 3H), 6.71-6.63 (m, 2H), 6.55-6.44 (m, 1H), 5.52 (s, 1H), 4.52-4.49 (m, 2H), 4.40-4.35 (m, 1H), 4.31-4.26 (m, 2H), 3.94-3.78 (m, 4H), 3.73-3.69 (m, 4H), 3.63-3.59 (m, 4H), 3.57-3.55 (m, 2H), 3.49-3.47 (m, 2H), 3.38-3.29 (m, 2H), 3.11-3.05 (m, 1H), 2.82-2.75 (m, 6H), 2.50-2.43 (m, 4H), 2.09-2.02 (m, 1H), 2.00-1.99 (m, 2H), 1.89-1.85 (m, 1H), 1.70-1.65 (m, 3H), 1.12 (s, 2H), 1.09 (s, 2H), 1.08-1.07 (m, 1H), 1.06-1.05 (m, 1H), 0.99-0.95 (m, 2H), 0.68-0.65 (m, 2H). LC-MS: MS (ES⁺): RT=2.355 min, m/z=1062.4 [M+H⁺], LCMS method 05.

### Example 56—the Synthetic Route for I-77

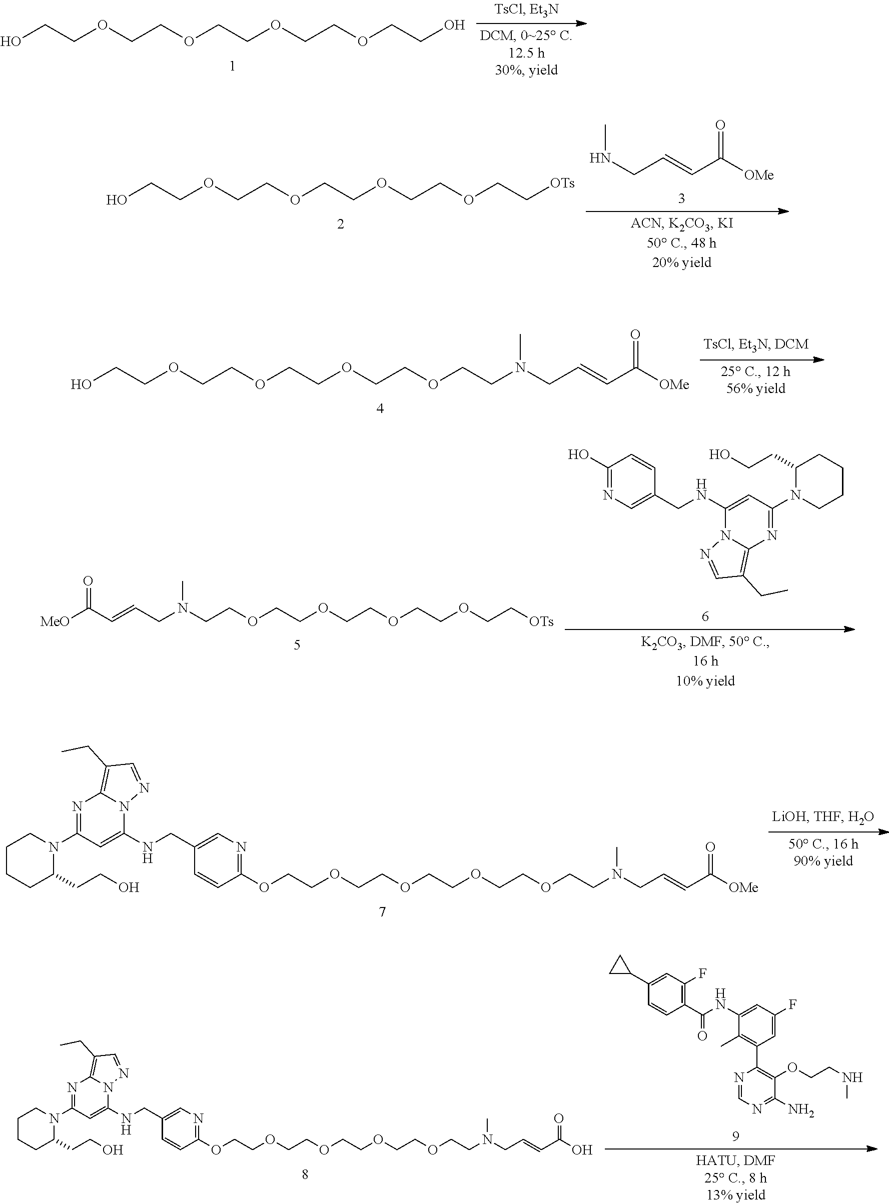

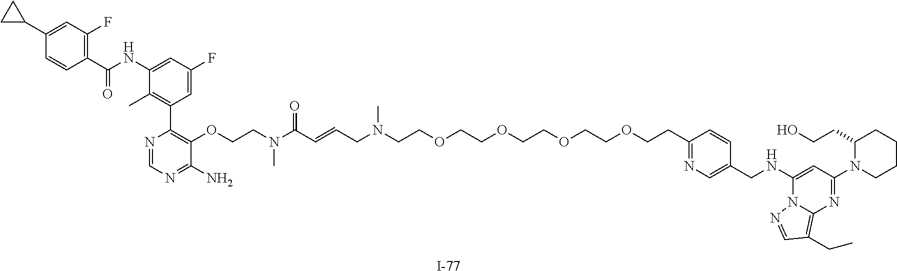

General Information: Synthetic route for compound 9 is described in US2015/152068, 2015, A1.

Step 1. To a solution of 4-methylbenzenesulfonyl chloride (16.0 g, 83.9 mmol, 1.0 equiv), TEA (25.5 g, 251 mmol, 35.1 mL, 3.0 equiv) in DCM (300 mL) was added dropwise 2-[2-[2-[2-(2-hydroxyethoxy)ethoxy]ethoxy]ethoxy]ethanol (20.0 g, 83.9 mmol, 17.9 mL, 1.0 equiv) at 0° C. After addition, the mixture was stirred at 0° C. for 0.5 h. The resulting mixture was stirred at 25° C. for 12 h. The reaction mixture was concentrated under reduced pressure to remove solvent. The residue was purified by column chromatography (SiO₂, Petroleum ether/Ethyl acetate=1/2 to 0/1) to give 2-[2-[2-[2-(2-hydroxyethoxy)ethoxy]ethoxy]ethoxy]ethyl 4-methylbenzenesulfonate (10.0 g, 25.5 mmol, 30% yield) as a colorless oil. ¹H NMR: (400 MHZ, CDCl₃) δ 7.79 (d, J=8.4 Hz, 2H), 7.35 (d, J=8.8 Hz, 2H), 4.19-4.06 (m, 2H), 3.73-3.67 (m, 4H), 3.67-3.55 (m, 16H), 2.45 (s, 3H).

Step 2. To a solution of 2-[2-[2-[2-(2-hydroxyethoxy)ethoxy]ethoxy]ethoxy]ethyl 4-methylbenzenesulfonate (8.4 g, 21.5 mmol, 1.5 equiv) and methyl (E)-4-(methylamino)but-2-enoate (3.5 g, 14.4 mmol, 1.0 equiv) in ACN(30 mL) was added K₂CO₃ (8.0 g, 57.6 mmol, 4.0 equiv) and KI (2.4 g, 14.4 mmol, 1.0 equiv). The mixture was stirred at 50° C. for 48 h. The reaction mixture was concentrated under reduced pressure to remove solvent. The residue was purified by prep-HPLC (column: YMC Triart C18 250×50 mm×7 μm; mobile phase: [water (NH₄HCO₃)-ACN]; B %: 13%-43%,20 min) to give methyl (E)-4-[2-[2-[2-[2-(2-hydroxyethoxy) ethoxy]ethoxy]ethoxy]ethyl-methyl-amino]but-2-enoate (1.0 g, 2.9 mmol, 20% yield) as a white oil.

Step 3. To a solution of 4-methylbenzenesulfonyl chloride (1.0 g, 5.4 mmol, 2.3 equiv), TEA (728 mg, 7.2 mmol, 1.0 mL, 3.1 equiv) in DCM (10 mL) was added dropwise methyl (E)-4-[2-[2-[2-[2-(2-hydroxyethoxy)ethoxy]ethoxy]ethoxy]ethyl-methyl-amino]but-2-enoate (800 mg, 2.3 mmol, 1.0 equiv) at 0° C. After addition, the mixture was stirred at this temperature for 0.5 h. The resulting mixture was stirred at 25° C. for 12 h. The reaction mixture was concentrated under reduced pressure to remove solvent. The residue was purified by flash silica gel chromatography (ISCO®; 12 g SepaFlash® Silica Flash Column, Eluent of 0˜6% DCM/MeOH at 40 mL/min) to give methyl (E)-4-[methyl-[2-[2-[2-[2-[2-(p-tolylsulfonyloxy)ethoxy]ethoxy]ethoxy]ethoxy]ethyl]amino]but-2-enoate (640 mg, 1.3 mmol, 56% yield) as a black brown oil. ¹H NMR: (400 MHZ, CDCl₃) δ=7.79 (d, J=6.8 Hz, 2H), 7.36 (d, J=7.6 Hz, 2H), 7.05-6.89 (m, 1H), 6.01 (d, J=16 Hz, 1H), 3.80-3.72 (m, 3H), 3.70-3.49 (m, 18H), 3.22 (d, J=4.8 Hz, 2H), 2.62 (s, 2H), 2.45 (s, 3H), 2.33 (s, 3H).

Step 4. A mixture of methyl (E)-4-[methyl-[2-[2-[2-[2-[2-(p-tolylsulfonyloxy) ethoxy]ethoxy]ethoxy]ethoxy]ethyl]amino]but-2-enoate (600 mg, 1.2 mmol, 1.5 equiv), 5-[[[3-ethyl-5-[(2S)-2-(2-hydroxyethyl)-1-piperidyl]pyrazolo[1,5-a]pyrimidin-7-yl]amino]methyl]pyridin-2-ol (315 mg, 794 μmol, 1.0 equiv), K₂CO₃ (329 mg, 2.4 mmol, 3.0 equiv) in DMF (8.0 mL) was degassed and purged with N₂ for 3 times, and then the mixture was stirred at 50° C. for 16 h. The reaction mixture was filtered and concentrated under reduced pressure to give a residue. The residue was purified by prep-HPLC (column: YMC Triart C18 70×250 mm×7 μm; mobile phase: [water (NH₄HCO₃)-ACN]; B %: 30%-60%,20 min), to give methyl (E)-4-[2-[2-[2-[2-[2-[[5-[[[3-ethyl-5-[2-(2-hydroxyethyl)-1-piperidyl]pyrazolo[1,5-a]pyrimidin-7-yl]amino]methyl]-2-pyridyl]oxy lethoxy]ethoxy]ethoxy]ethoxy]ethyl-methyl-amino]but-2-enoate (62 mg, 85 μmol, 10% yield) as a yellow oil.

Step 5. To a solution of methyl (E)-4-[2-[2-[2-[2-[2-[5-[[[3-ethyl-5-[2-(2-hydroxyethyl)-1-piperidyl]pyrazolo[1,5-a]pyrimidin-7-yl]amino]methyl]-2-pyridyl Joxy]ethoxy]ethoxy]ethoxy]ethoxy]ethyl-methyl-amino]but-2-enoate (62 mg, 85 μmol, 1.0 equiv) in THF (1.0 mL) was added LiOH·H₂O (5 mg, 128 μmol, 1.5 equiv) and H₂O (0.3 mL). The mixture was stirred at 50° C. for 16 h. The reaction mixture was filtered and concentrated under reduced pressure to give (E)-4-[2-[2-[2-[2-[2-[5-[[[3-ethyl-5-[2-(2-hydroxyethyl)-1-piperidyl]pyrazolo[1,5-a]pyrimidin-7-yl]amino]methyl]-2-pyridyl Joxy]ethoxy]ethoxy]ethoxy]ethoxy]ethyl-methyl-amino]but-2-enoic acid (55 mg, 77 μmol, 90% yield) as a yellow oil.

Step 6. A mixture of (E)-4-[2-[2-[2-[2-[2-[[5-[[[3-ethyl-5-[2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]ethyl-methyl-amino]but-2-enoic acid (60 mg, 85 μmol, 1.0 equiv), N-[3-[6-amino-5-[2-(methylamino)ethoxy]pyrimidin-4-yl]-5-fluoro-2-methyl-phenyl]-4-cyclopropyl-2-fluoro-benzamide (48 mg, 85 μmol, 1.0 equiv, TFA), HATU (49 mg, 127 μmol, 1.5 equiv), DIEA (33 mg, 255 μmol, 44 μL, 3.0 equiv) in DMF (2.0 mL) was degassed and purged with N₂ for 3 times, and then the mixture was stirred at 25° C. for 8 h. The reaction mixture was filtered and concentrated under reduced pressure to give a residue. The residue was purified by prep-HPLC (column: Phenomenex C18 150×25 mm×10 μm; mobile phase: [water (FA)-ACN]; B %: 15%-45%,5 min), to give N-[3-[6-amino-5-[2-[[(E)-4-[2-[2-[2-[2-[2-[5-[[[3-ethyl-5-[(2S)-2- (2-hydroxyethyl)-1-piperidyl]pyrazolo[1,5-a]pyrimidin-7-yl]amino]methyl]-2-pyridyl]oxy]ethoxy]ethoxy]ethoxy]ethoxy]ethyl-methyl-amino]but-2-enoyl]-methyl-amino]ethoxy]pyrimidin-4-yl]-5-fluoro-2-methyl-phenyl]-4-cyclopropyl-2-fluoro-benzamide (14 mg, 11 μmol, 13% yield, 98% purity, FA) as a white solid. ¹H NMR: (400 MHZ, CD₃OD) δ 8.48 (s, 1H), 8.19 (s, 2H), 7.87-7.73 (m, 2H), 7.69-7.61 (m, 2H), 7.52-7.31 (m, 1H), 7.06 (d, J=8.4 Hz, 1H), 7.02-6.95 (m, 2H), 6.80 (d, J=2.4, 8.6 Hz, 1H), 6.76-6.71 (m, 1H), 6.70-6.58 (m, 1H), 5.53 (s, 1H), 4.86 (s, 39H), 4.80 (d, J=3.2, 6.6 Hz, 1H), 4.53 (s, 2H), 4.41-4.35 (m, 2H), 4.12-3.98 (m, 1H), 3.85-3.78 (m, 2H), 3.76-3.70 (m, 1H), 3.74-3.47 (m, 23H), 3.45-3.39 (m, 1H), 3.39-3.35 (m, 1H), 3.08-2.92 (m, 3H), 2.89 (s, 2H), 2.87-2.80 (m, 1H), 2.63-2.58 (m, 3H), 2.55 (d, J=1.2, 7.6 Hz, 2H), 2.49 (s, 2H), 2.14-2.04 (m, 4H), 2.03-1.95 (m, 1H), 1.81-1.62 (m, 7H), 1.22 (t, J=7.6 Hz, 3H), 1.13-1.00 (m, 2H), 0.81-0.76 (m, 2H). LC-MS: MS (ES⁺): RT=2.295 min, m/z=575.6 [M/2+H+], LCMS method 40.

### Example 57—the Synthetic Route for I-78

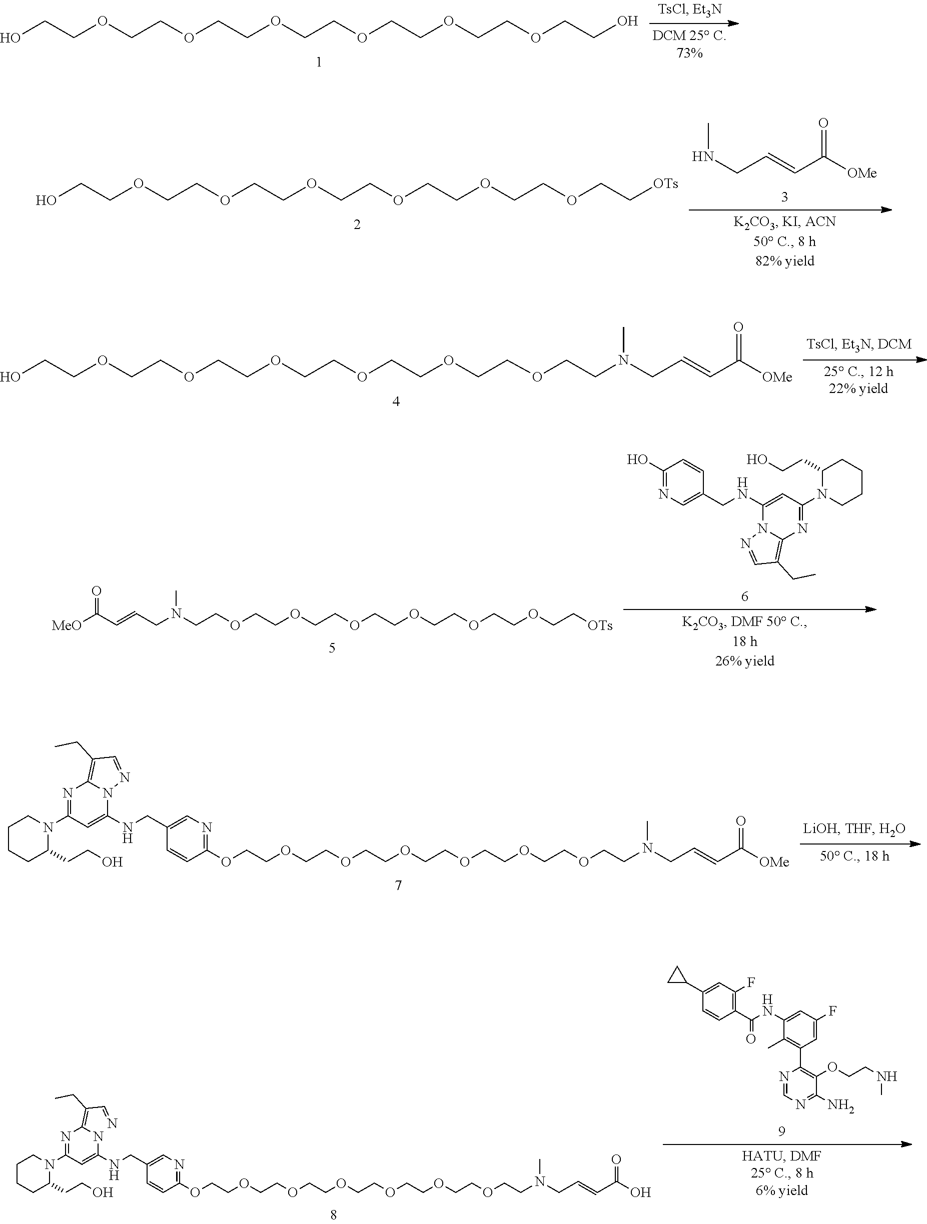

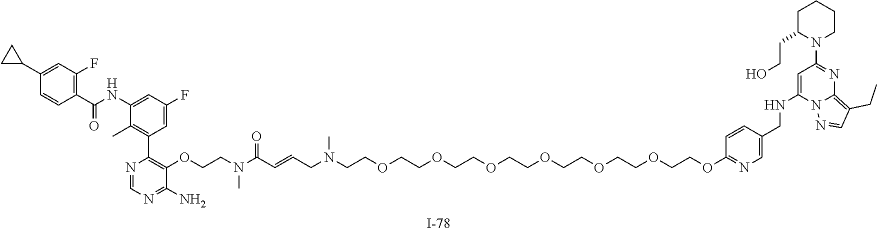

General Information: Synthetic route for compound 9 is described in US2015/152068.

Step 1. To a solution of 4-methylbenzenesulfonyl chloride (8.8 g, 46.0 mmol, 1.0 equiv), TEA (14.0 g, 137 mmol, 19.2 mL, 3.0 equiv) in DCM (200 mL) was added dropwise 2-[2-[2-[2-[2-[2-(2-hydroxyethoxy)ethoxy]ethoxy]ethoxy]ethoxy]ethoxy]ethanol (15.0 g, 45.9 mmol, 17.9 mL, 1.0 equiv) at 0° C. After addition, the mixture was stirred at this temperature for 0.5 h. The resulting mixture was stirred at 25° C. for 3 h. The reaction mixtures was concentrated under reduced pressure to remove solvent. The residue was purified by column chromatography (SiO₂, Petroleum ether/Ethyl acetate=1/2 to 0/1) to give the 2-[2-[2-[2-[2-[2-(2-hydroxyethoxy)ethoxy]ethoxy]ethoxy]ethoxy]ethoxy]ethyl 4-methylbenzenesulfonate (16.1 g, 33.5 mmol, 73% yield) as a white oil. ¹H NMR: (400 MHZ, CDCl₃) δ=7.78-7.76 (d, J=8.4 Hz, 2H), 7.37-7.32 (d, J=10.4 Hz, 2H), 4.24-4.10 (m, 2H), 3.75-3.70 (m, 3H), 3.67-3.61 (m, 22H), 2.48-2.40 (s, 3H).

Step 2. To a solution of 2-[2-[2-[2-[2-[2-(2-hydroxyethoxy)ethoxy]ethoxy]ethoxy]ethoxy]ethoxy]ethyl 4-methylbenzenesulfonate (4.0 g, 8.3 mmol, 1.0 equiv) in ACN(35 mL) was added K₂CO₃ (3.5 g, 24.9 mmol, 3.0 equiv) and KI (1.4 g, 8.3 mmol, 1.0 equiv), (E)-methyl 4-(methylamino)but-2-enoate (2.0 g, 8.3 mmol, 1.0 equiv). The mixture was stirred at 50° C. for 8 h. The reaction mixture was poured into H₂O (200 mL) and extracted with DCM (150 mL×3). The combined organic phase was concentrated in vacuo to give a residue. The residue was purified by column chromatography (SiO₂, DCM: MeOH=20:1-10:1). Compound methyl (E)-4-[2-[2-[2-[2-[2-[2-(2-hydroxyethoxy)ethoxy]ethoxy]ethoxy]ethoxy]ethoxy]ethyl-methyl-amino]but-2-enoate (3.0 g, 6.9 mmol, 82% yield) was obtained as a yellow oil. 1H NMR: (400 MHZ, CDCl₃) δ=6.98-6.90 (d, J=14.4 Hz, 6.4 Hz, 1H), 6.05-5.95 (d, J=15.6 Hz, 1H), 3.74 (m, 2H), 3.73-3.71 (m, 2H), 3.71 (m, 3H), 3.65 (m, 4H), 3.63 (m, 4H), 3.63 (s, 4H), 3.61-3.60 (m, 4H), 3.60-3.58 (m, 3H), 3.33-3.29 (m, 2H), 3.28-3.24 (m, 1H), 2.71-2.66 (m, 2H), 2.34 (s, 3H), 1.99 (m, 4H).

Step 3. To a solution of (E)-methyl 1-hydroxy-21-methyl-3,6,9,12,15,18-hexaoxa-21-azapentacos-23-en-25-oate (1.0 g, 2.3 mmol, 1.0 equiv) in DCM (10 mL) was added TsCl (434 mg, 2.3 mmol, 1.0 equiv) and TEA (690 mg, 6.8 mmol, 3.0 equiv). The mixture was stirred at 25° C. for 12 h. The reaction mixture was concentrated under reduced pressure to remove solvent. The residue was diluted with H₂O (50 mL) and extracted with EA (50 mL×3). The combined organic layers were washed with water (50 mL×2), dried over Na₂SO₄ filtered and concentrated under reduced pressure to give a residue. The residue was purified by column chromatography (SiO₂, DCM/MeOH=20/1 to 10/1). Compound (E)-methyl 21-methyl-1-(tosyloxy)-3,6,9,12, 15, 18-hexaoxa-21-azapentacos-23-en-25-oate (300 mg, 508 μmol, 22% yield) was obtained as a yellow oil. ¹H NMR (400 MHZ, CDCl₃) δ=7.85-7.74 (d, J=8.4 Hz, 2H), 7.41-7.32 (d, J=8.4 Hz, 2H), 7.01-6.92 (m, 1H), 6.08-5.93 (m, 1H), 3.74 (s, 3H), 3.70-3.59 (m, 26H), 3.28-3.20 (m, 2H), 2.67-2.58 (m, 2H), 2.48-2.43 (s, 3H), 2.33-2.27 (s, 3H).

Step 4. To a solution of (E)-methyl 21-methyl-1-(tosyloxy)-3,6,9,12,15,18-hexaoxa-21-azapentacos-23-en-25-oate (210 mg, 355 μmol, 1.5 equiv) in DMF (2 mL) was added K₂CO₃ (98 mg, 709 μmol, 3.0 equiv) and(S)-5-(((3-ethyl-5-(2-(2-hydroxyethyl) piperidin-1-yl) pyrazolo[1,5-a]pyrimidin-7-yl)amino) methyl) pyridin-2-ol (94 mg, 237 μmol, 1.0 equiv). The mixture was stirred at 50° C. for 18 h. The reaction mixture was diluted with H₂O 20 mL and extracted with EA 60 mL (20 mL×3). The combined organic layers were washed with H₂O (25 mL×2), dried over Na₂SO₄, filtered and concentrated under reduced pressure to give a residue. The crude product was purified by reversed-phase HPLC column: Waters xbridge 150*25 mm 10 μm; mobile phase: [water (NH₄OAc)-ACN]; B %: 28%-58%, 11 min). Compound (S,E)-methyl 1-(5-(((3-ethyl-5-(2-(2-hydroxyethyl) piperidin-1-yl) pyrazolo[1,5-a]pyrimidin-7-yl)amino) methyl) pyridin-2-yl)-21-methyl-3,6,9,12,15,18-hexaoxa-21-azapentacos-23-en-25-oate (50 mg, 61 μmol, 26% yield) was obtained as a light yellow oil. ¹H NMR: (400 MHZ, CDCl₃) δ=7.83-7.78 (m, 1H), 7.66-7.63 (m, 1H), 7.55-7.51 (m, 1H), 7.41-7.34 (m, 2H), 7.02-6.96 (m, 1H), 6.61-6.59 (d, J=9.2 Hz, 1H), 6.36-6.29 (m, 1H), 6.03-5.99 (d, J=15.6 Hz, 1.2 Hz, 1H), 5.32 (s, 1H), 5.14-5.05 (d, J=12.4 Hz, 1H), 4.31-4.26 (m, 2H), 4.17-4.14 (m, 2H), 3.80-3.77 (m, 3H), 3.75-3.75 (m, 3H), 3.66-3.65 (d, J=1.6 Hz, 4H), 3.63-3.63 (m, 4H), 3.43-3.32 (m, 2H), 3.24-3.22 (m, 2H), 3.09-3.02 (m, 1H), 2.64-2.60 (m, 4H), 2.50-2.44 (m, 2H), 2.31-2.29 (m, 4H), 2.14-1.90 (m, 10H), 1.78-1.64 (m, 6H), 1.58-1.51 (m, 1H), 1.30-1.23 (m, 4H).

Step 5. To a solution of (S,E)-methyl 1-(5-(((3-ethyl-5-(2-(2-hydroxyethyl) piperidin-1-yl) pyrazolo[1,5-a]pyrimidin-7-yl)amino) methyl) pyridin-2-yl)-21-methyl-3,6,9,12,15,18-hexaoxa-21-azapentacos-23-en-25-oate (40 mg, 49 μmol, 1.0 equiv) in THF (1 mL) and H₂O (0.3 mL) was added LiOH·H₂O (3 mg, 74 μmol, 1.5 equiv). The mixture was stirred at 50° C. for 18 h. The reaction mixture was concentrated under reduced pressure to give a residue and used into the next step without further purification. Compound (S,E)-1-(5-(((3-ethyl-5-(2-(2-hydroxyethyl) piperidin-1-yl) pyrazolo[1,5-a]pyrimidin-7-yl)amino) methyl) pyridin-2-yl)-21-methyl-3,6,9,12, 15, 18-hexaoxa-21-azapentacos-23-en-25-oic acid (39 mg, 49 μmol,) was obtained as a light yellow solid.

Step 6. To a solution of N-(3-(6-amino-5-(2-(methylamino)ethoxy) pyrimidin-4-yl)-5-fluoro-2-methylphenyl)-4-cyclopropyl-2-fluorobenzamide (30 mg, 66 μmol, 1.0 equiv) in DMF (1 mL) was added HATU (38 mg, 99 μmol, 1.5 equiv) and DIEA (26 mg, 198 μmol, 35 μL, 3.0 equiv), (S,E)-1-(5-(((3-ethyl-5-(2-(2-hydroxyethyl) piperidin-1-yl) pyrazolo[1,5-a]pyrimidin-7-yl)amino) methyl) pyridin-2-yl)-21-methyl-3,6,9,12,15,18-hexaoxa-21-azapentacos-23-en-25-oic acid (53 mg, 66 μmol, 1.0 equiv). The mixture was stirred at 25° C. for 8 h. The reaction mixture was diluted with H₂O (20 mL) and extracted with EA (20 mL×3). The combined organic layers were washed with H₂O (25 mL×2), dried over Na₂SO₄, filtered and concentrated under reduced pressure to give a residue. The crude product was purified by reversed-phase HPLC column: Phenomenex C18 150*25 mm*10 μm; mobile phase: [water (NH₄HCO₃)-ACN]; B %: 43%-73%,5 min. Compound (S,E)-N-(2-((4-amino-6-(3-(4-cyclopropyl-2-fluorobenzamido)-5-fluoro-2-methylphenyl)pyrimidin-5-yl)oxy) ethyl)-1-(5-(((3-ethyl-5-(2-(2-hydroxyethyl) piperidin-1-yl) pyrazolo[1,5-a]pyrimidin-7-yl)amino) methyl) pyridin-2-yl)-N,21-dimethyl-3,6,9,12,15,18-hexaoxa-21-azapentacos-23-en-25-amide (5 mg, 4 μmol, 6% yield) was obtained as a light yellow oil. ¹H NMR: (400 square MHz, CD₃OD) δ=8.25-8.17 (d, J=14.0 Hz, 2H), 7.90-7.81 (m, 1H), 7.80-7.74 (m, 1H), 7.72-7.63 (m, 2H), 7.08 (d, J=8.4 Hz, 1H), 7.05-6.97 (m, 2H), 6.84-6.69 (m, 2H), 6.68-6.44 (m, 1H), 5.4 (s, 1H), 4.84-4.79 (m, 2H), 4.54 (s, 2H), 4.46-4.40 (m, 2H), 4.08-4.01 (m, 1H), 3.89-3.82 (m, 2H), 3.74-3.62 (m, 8H), 3.60 (s, 14H), 3.46-3.35 (m, 2H), 3.28-3.20 (m, 2H), 3.06-2.88 (m, 3H), 2.64-2.49 (m, 6H), 2.30-2.24 (d, J=8.8 Hz, 3H), 2.17-1.92 (m, 6H), 1.66 (s, 6H), 1.59-1.31 (m, 2H), 1.28-1.21 (m, 4H), 1.14-1.08 (m, 2H), 0.86-0.75 (m, 2H). LC-MS: MS (ES⁺): RT=2.329 min, m/z=619.8 [1/2 M+H⁺], LCMS method 05.

### Example 58—the Synthetic Route for I-79

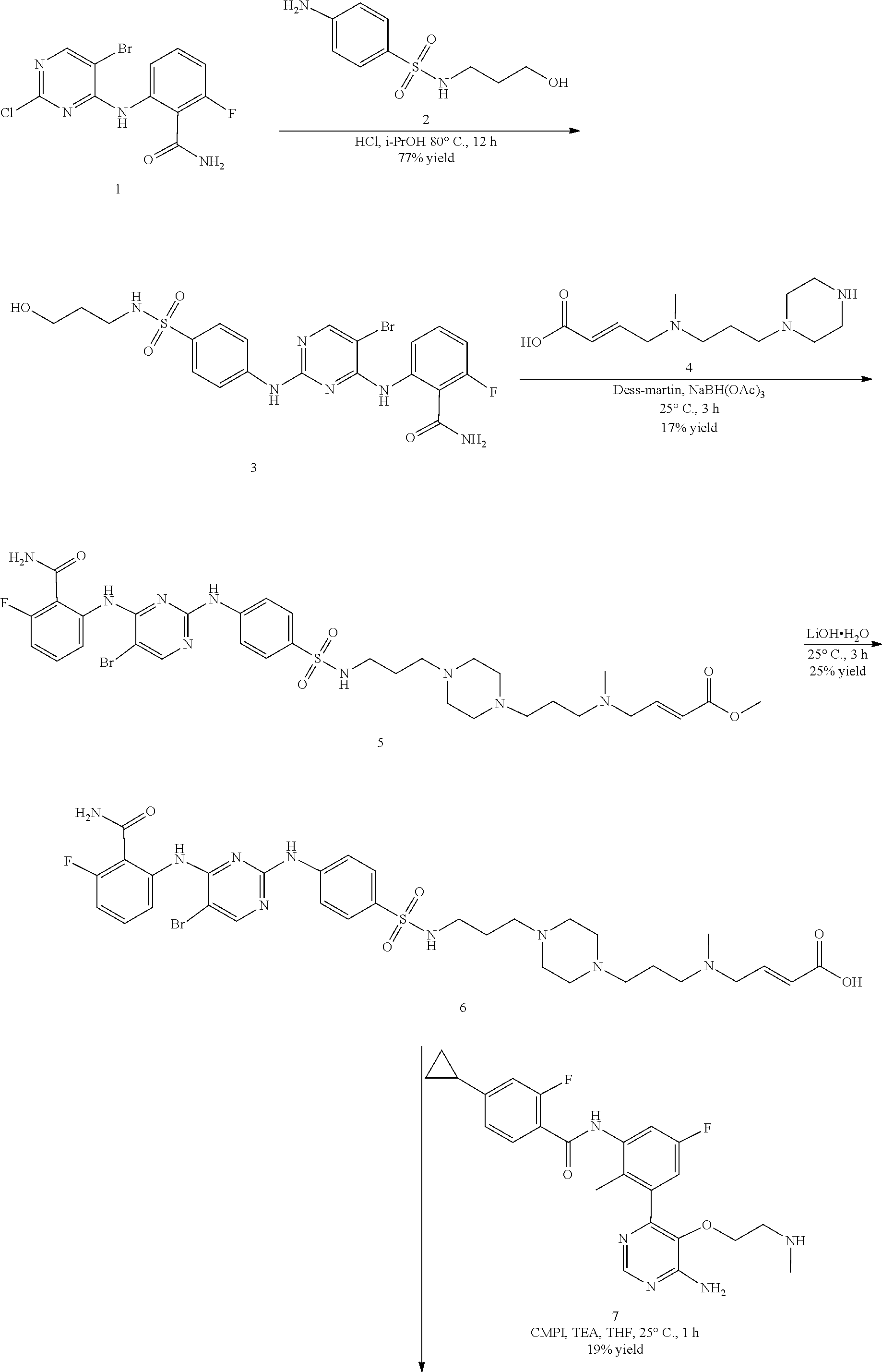

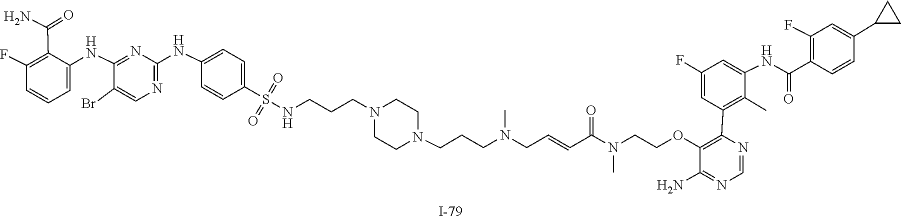

General Information: Synthetic route for compound 1 is described in WO2020/206137, 2020, A¹. Synthetic route for compound 2 is described in Journal of the American Chemical Society, 1939, 61, 2342. Synthetic route for compound 7 is described in WO2015/79417, 2015, A¹

Step 1. A mixture of 2-[(5-bromo-2-chloro-pyrimidin-4-yl)amino]-6-fluoro-benzamide (500 mg, 1.5 mmol, 1.0 equiv), 4-amino-N-(3-hydroxypropyl) benzenesulfonamide (400 mg, 1.7 mmol, 1.2 equiv), HCl (12 M, 12 μL, 0.1 equiv) in i-PrOH (5 mL) was degassed and purged with N₂ for 3 times, and then the mixture was stirred at 80° C. for 12 h under N₂ atmosphere. The mixture was cooled to 25° C. and filtered, the filtrate was collected to give the 2-[[5-bromo-2-[4-(3-hydroxypropylsulfamoyl) anilino]pyrimidin-4-yl]amino]-6-fluoro-benzamide (600 mg, 1.11 mmol, 77% yield) as a white solid. ¹H NMR: (400 MHZ, DMSO-d₆) δ=10.18-10.01 (m, 2H), 8.40 (s, 1H), 8.19 (d, J=8.4 Hz, 1H), 8.13 (d, J=16.0 Hz, 2H), 7.82 (d, J=8.8 Hz, 2H), 7.62 (d, J=8.8 Hz, 2H), 7.58-7.49 (m, 1H), 7.35 (s, 1H), 7.14-7.05 (m, 1H), 3.38-3.33 (m, 2H), 2.79-2.72 (m, 2H), 1.56-1.46 (m, 2H).

Step 2. To a mixture of 2-[[5-bromo-2-[4-(3-hydroxypropylsulfamoyl) anilino]pyrimidin-4-yl]amino]-6-fluoro-benzamide (800 mg, 1.5 mmol, 1.0 equiv) in DMF (8 mL) was added Dess-Martin (1.6 g, 3.7 mmol, 2.5 equiv) and stirred for 1 h at 25° C. Then the mixture was added TEA to adjusted to pH=6-7. The mixture was added methyl (E)-4-[methyl (3-piperazin-1-ylpropyl)amino]but-2-enoate (657 mg, 1.8 mmol, 1.2 equiv, TFA) and stirred for 30 min and the NaBH (OAc)3 (3.1 g, 14.8 mmol, 10.0 equiv) was added to the mixture and stirred for 2 h at 25° C. The mixture was filtered and concentrated to give the residue and was purification by the prep-HPLC (column: Phenomenex Luna C18 200*40 mm*10 μm; mobile phase: [water (TFA)-ACN]; B %: 10%-40%, 10 min) to give the methyl (E)-4-[3-[4-[3-[[4-[[5-bromo-4-(2-carbamoyl-3-fluoro-anilino) pyrimidin-2-yl]amino]phenyl]sulfonylamino]propyl]piperazin]yl]propyl methylamino]but-2-enoate (200 mg, 257 μmol, 17% yield) as a yellow solid. ¹H NMR: (400 MHZ, DMSO-d₆) δ=8.46-8.04 (m, 4H), 7.96-7.51 (m, 6H), 7.18-7.03 (m, 1H), 6.94-6.77 (m, 1H), 6.34 (d, J=15.6 Hz, 1H), 3.98 (d, J=6.8 Hz, 4H), 3.29-2.94 (m, 9H), 2.88-2.72 (m, 6H), 2.11-1.70 (m, 5H).

Step 3. A mixture of methyl (E)-4-[3-[4-[3-[[4-[[5-bromo-4-(2-carbamoyl-3-fluoro-anilino) pyrimidin-2-yl]amino]phenyl]sulfonylamino]propyl]piperazin-1-yl]propyl-methyl-amino]but-2-enoate (200 mg, 257 μmol, 1.0 equiv), LiOH·H₂O (22 mg, 515 μmol, 2.0 equiv) in THF (0.5 mL), H₂O (0.5 mL) was degassed and purged with N₂ for 3 times, and then the mixture was stirred at 25° C. for 2 h under N₂ atmosphere. The mixture was concentrated to get the residue. The residue was purified by the prep-HPLC (column: Phenomenex luna C18 150*25 mm*10 μm; mobile phase: [water (FA)-ACN]; B %: %-36%, 10 min) to give the (E)4[3[4[3[[4[[5bromo4 (2carbamoyl3fluoroanilino) pyrimidin2yl]amino]phenyl]sulfonylamino]propyl]piperazin-1-yl]propyl-methyl-amino]but-2-enoic acid (50 mg, 66 μmol, 25% yield) as a white solid.

Step 4. A mixture of (E)-4-[3-[4-[3-[[4-[[5-bromo-4-(2-carbamoyl-3-fluoro-anilino) pyrimidin-2-yl]amino]phenyl]sulfonylamino]propyl]piperazin-1-yl]propyl-methyl-amino]but-2-enoic acid (50 mg, 66 μmol, 1.0 equiv), N-[3-[6-amino-5-[2-(methylamino)ethoxy]pyrimidin-4-yl]-5-fluoro-2-methyl-phenyl]-4-cyclopropyl-2-fluoro-benzamide (30 mg, 66 μmol, 1.0 equiv), CMPI (25 mg, 98 μmol, 1.5 equiv), TEA (6 mg, 66 μmol, 1 equiv) in THF (1 mL) was degassed and purged with N₂ for 3 times, and then the mixture was stirred at 25° C. for 1 h under N₂ atmosphere. The mixture was purified by the prep-HPLC (column: Welch Xtimate C18 150*25 mm*5 μm; mobile phase: [water (FA)-ACN]; B %: 10%-40%, 10 min) to give the 2-[[2-[4-[3-[4-[3-[[(E)-4-[2-[4-amino-6-[3-[(4-cyclopropyl-2-fluoro-benzoyl)amino]-5-fluoro-2-methyl-phenyl]pyrimidin-5-yl]oxyethyl-methyl-amino]-4-oxo-but-2-enyl]-methyl-amino]propyl]piperazin-1-yl]propylsulfamoyl]anilino]-5-bromo-pyrimidin-4-yl]amino]-6-fluoro-benzamide (16 mg, 13 μmol, 19% yield, 97% purity) as a white solid. ¹H NMR: (400 MHZ, DMSO-d₆) δ=10.13 (s, 1H), 9.93 (s, 1H), 9.79-9.46 (m, 1H), 8.38 (s, 1H), 8.33-8.10 (m, 4H), 7.84 (d, J=8.8 Hz, 2H), 7.78-7.46 (m, 5H), 7.37 (d, J=4.0 Hz, 1H), 7.15-7.02 (m, 4H), 7.00-6.94 (m, 1H), 6.64-6.30 (m, 2H), 3.56 (d, J=5.2 Hz, 2H), 3.08-2.97 (m, 3H), 2.78 (s, 1H), 2.77-2.69 (m, 2H), 2.47 (s, 2H), 2.31-2.09 (m, 13H), 2.07 (s, 3H), 2.04-1.94 (m, 4H), 1.58-1.38 (m, 4H), 1.11-0.97 (m, 2H), 0.85-0.70 (m, 2H). LC-MS: MS (ES⁺): RT=2.250 min, m/z=599.5 [1/2M+H⁺]; LCMS method 05.

### Example 59—the Synthetic Route for I-80

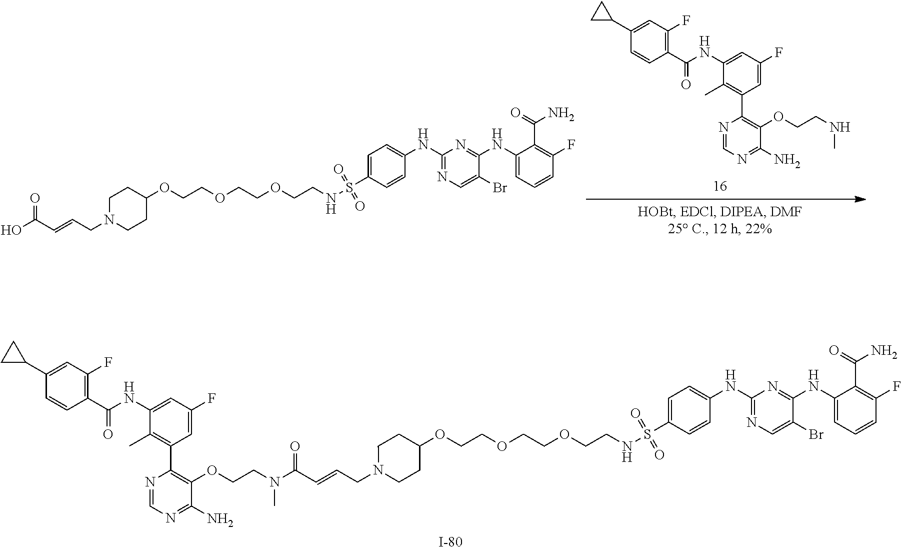

Step 1. To a solution of N-[3-[6-amino-5-[2-(methylamino)ethoxy]pyrimidin-4-yl]-5-fluoro-2-methyl-phenyl]-4-cyclopropyl-2-fluoro-benzamide (45 mg, 99 μmol, 1.0 equiv), (E)-4-[4-[2-[2-[2-[[4-[[5-bromo-4-(2-carbamoyl-3-fluoro-anilino) pyrimidin-2-yl]amino]phenyl]sulfonylamino]ethoxy]ethoxy]ethoxy]-1-piperidyl]but-2-enoic acid (77 mg, 99 μmol, 1.0 equiv), HOBt (17 mg, 129 μmol, 1.3 equiv), EDCI (25 mg, 129 μmol, 1.3 equiv) and DIPEA (26 mg, 198 μmol, 35 μL, 2.0 equiv) in DMF (1 mL). The mixture was stirred at 25° C. for 12 h. The reaction mixture was diluted with 5 mL H₂O and extracted with EtOAc (10 mL×3). The combined organic layers were washed with brine 10 mL, dried over Na₂SO₄, filtered and concentrated under reduced pressure to give a residue. The residue was purified by prep-HPLC (column: Phenomenex luna C18 150×25 mm×10 μm; mobile phase: [water (FA)-ACN]; B %: 20%-50%, 10.5 min) to afford 2-[[2-[4-[2-[2-[2-[[1-[(E)-4-[2-[4-amino-6-[3-[(4-cyclopropyl-2-fluoro-benzoyl)amino]-5-fluoro-2-methyl-phenyl]pyrimidin-5-yl]oxyethyl-methyl-amino]-4-oxo-but-2-enyl]-4-piperidyl]oxy]ethoxy]ethoxy]ethylsulfamoyl]anilino]-5-bromo-pyrimidin-4-yl]amino]-6-fluoro-benzamide (28 mg, 22.0 μmol, 22% yield, 95% purity) as a white solid. 1H NMR: (400 MHZ, CD₃OD) δ=8.46 (s, 1H), 8.36 (d, J=8.4 Hz, 1H), 8.27-8.17 (m, 2H), 7.84-7.76 (m, 3H), 7.72-7.63 (m, 3H), 7.51-7.45 (m, 1H), 7.04 (d, J=2.8, 8.0 Hz, 1H), 7.01-6.93 (m, 3H), 6.76-6.66 (m, 1H), 6.65-6.55 (m, 1H), 3.74-3.69 (m, 1H), 3.63 (s, 1H), 3.59 (d, J=8.4 Hz, 6H), 3.53-3.45 (m, 6H), 3.06-2.95 (m, 4H), 2.88 (s, 2H), 2.79-2.65 (m, 2H), 2.61 (s, 2H), 2.14-1.70 (m, 9H), 1.11-1.05 (m, 2H), 0.82-0.76 (m, 2H). LC-MS: MS (ES⁺): RT=2.476 min, m/z=1217.1 [M+H⁺], LCMS method 05.

### Example 60—the Synthetic Route for I-81

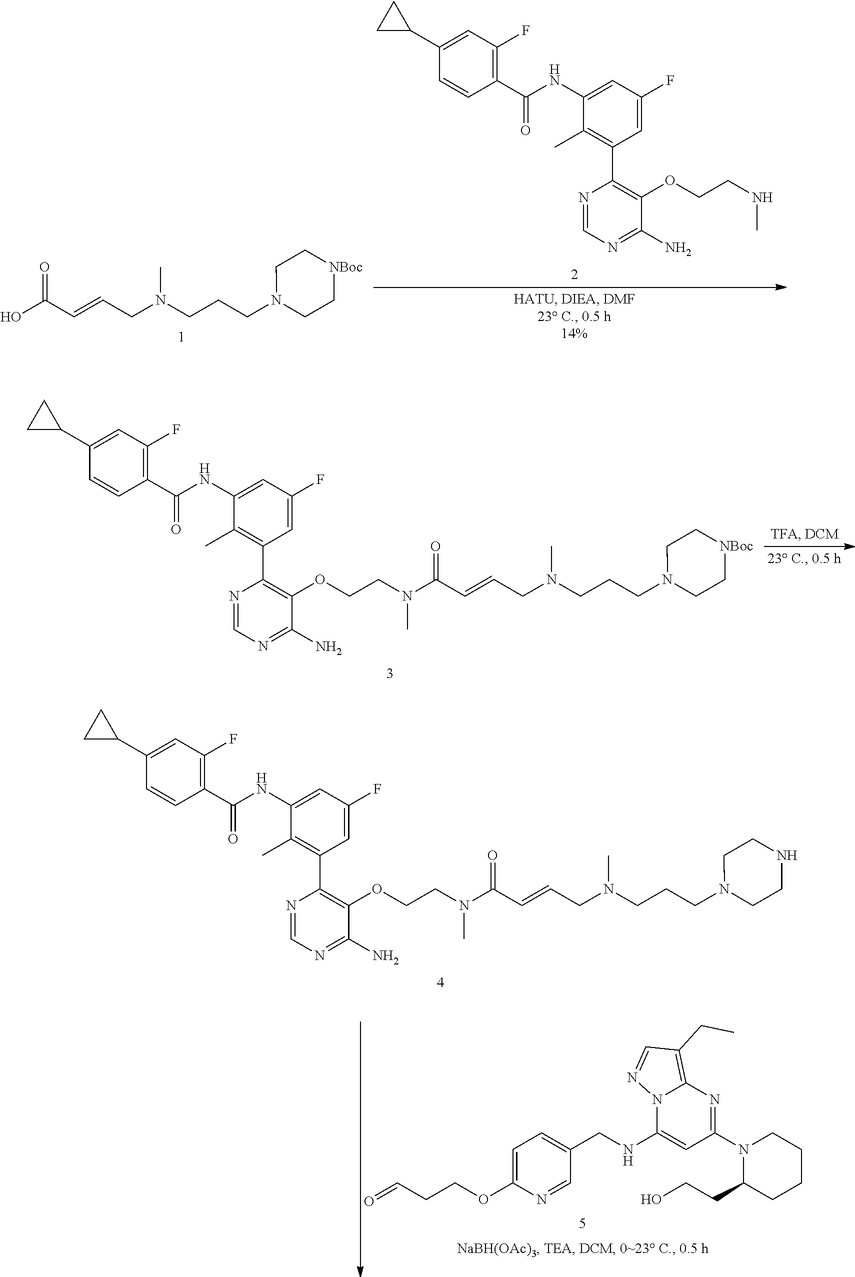 [SMILES: CNCCOc1c(N)ncnc1-c1cc(F)cc(NC(=O)c2ccc(C3CC3)cc2F)c1C ; CN(C/C=C/C(=O)O)CCCN1CCN(C(=O)OC(C)(C)C)CC1 ; Cc1c(NC(=O)c2ccc(C3CC3)cc2F)cc(F)cc1-c1ncnc(N)c1OCCN(C)C(=O)/C=C/CN(C)CCCN1CCN(C(=O)OC(C)(C)C)CC1 ; Cc1c(NC(=O)c2ccc(C3CC3)cc2F)cc(F)cc1-c1ncnc(N)c1OCCN(C)C(=O)/C=C/CN(C)CCCN1CCNCC1 ; CCc1cnn2c(NCc3ccc(OCCC=O)nc3)cc(N3CCCC[C@H]3CCO)nc12]

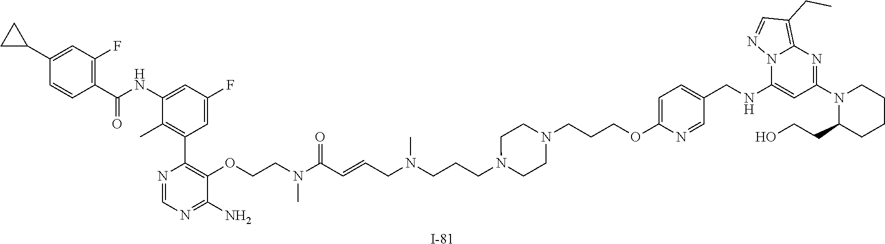

General Information: Synthetic route for compound 2 is described in US2015/152068.

Step 1. To a solution of [(E)-4-[3-(4-tert-butoxycarbonylpiperazin-1-yl) propyl-methyl-amino]but-2-enoyl]oxylithium (97 mg, 279 μmol, 1.0 equiv) in DMF (2 mL) was added a solution of N-[3-[6-amino-5-[2-(methylamino)ethoxy]pyrimidin-4-yl]-5-fluoro-2-methyl-phenyl]-4-cyclopropyl-2-fluoro-benzamide (158 mg, 279 μmol, 1.0 equiv, TFA) in DMF (2 mL) was added DIEA (180 mg, 1.4 mmol, 243 μL, 5.0 equiv) to adjust pH about 7˜8 and was added HATU (159 mg, 418 μmol, 1.5 equiv). The mixture was stirred at 23° C. for 0.5 h. The mixture was purified by prep-HPLC (column: Waters xbridge 150*25 mm 10 μm; mobile phase: [water (NH₄HCO₃)-ACN]; B %: 32%-62%, 8 min) to afford tert-butyl 4-[3-[[(E)-4-[2-[4-amino-6-[3-[(4-cyclopropyl-2-fluoro-benzoyl)amino]-5-fluoro-2-methyl-phenyl]pyrimidin-5-yl]oxyethyl-methyl-amino]-4-oxo-but-2-enyl]-methyl-amino]propyl]piperazine-1-carboxylate (33 mg, 41 μmol, 14% yield, 97% purity) as a white solid. ¹H NMR: (400 MHZ, CDCl₃) δ 8.57 (d, J=17.6 Hz, 1H), 8.34 (s, 1H), 8.18-8.04 (m, 2H), 7.04 (d, J=8.4 Hz, 1H), 7.00-6.94 (m, 1H), 6.94-6.83 (m, 2H), 6.46-6.36 (m, 1H), 6.02-5.82 (m, 1H), 3.60 (s, 3H), 3.43-3.42 (m, 4H), 3.15 (d, J=6.0 Hz, 2H), 2.98 (s, 2H), 2.84 (s, 1H), 2.44-2.29 (m, 8H), 2.24 (s, 3H), 2.19-2.13 (m, 3H), 2.02-1.92 (m, 1H), 1.72-1.64 (m, 2H), 1.47 (s, 9H), 1.31-1.24 (m, 1H), 1.17-1.09 (m, 2H), 0.85-0.77 (m, 2H).

Step 2. To a solution of tert-butyl 4-[3-[[(E)-4-[2-[4-amino-6-[3-[(4-cyclopropyl-2-fluoro-benzoyl)amino]-5-fluoro-2-methyl-phenyl]pyrimidin-5-yl]oxyethyl-methyl-amino]-4-oxo-but-2-enyl]-methyl-amino]propyl]piperazine-1-carboxylate (30 mg, 38 μmol, 1.0 equiv) in DCM (1.5 mL) was added TFA (462 mg, 4.1 mmol, 0.3 mL). The mixture was stirred at 23° C. for 0.5 h. The reaction mixture was concentrated under reduced pressure to remove solvent to afford N-[3-[6-amino-5-[2-[methyl-[(E)-4-[methyl (3-piperazin-1-ylpropyl)amino]but-2-enoyl]amino]ethoxy]pyrimidin-4-yl]-5-fluoro-2-methyl-phenyl]-4-cyclopropyl-2-fluoro-benzamide (31 mg crude, TFA) was used into the next step.

Step 3. To a solution of N-[3-[6-amino-5-[2-[methyl-[(E)-4-[methyl (3-piperazin-1-ylpropyl)amino]but-2-enoyl]amino]ethoxy]pyrimidin-4-yl]-5-fluoro-2-methyl-phenyl]-4-cyclopropyl-2-fluoro-benzamide (31 mg, 39 μmol, 1.0 equiv, TFA) in DCM (1 mL) was added TEA (4.0 mg, 39 μmol, 5 μL, 1.0 equiv) to adjust pH about 7˜8 at 0° C., NaBH (OAc)3 (82 mg, 386 μmol, 10.0 equiv) and 3-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]propanal (18 mg, 39 μmol, 1.0 equiv) in DCM (1 mL) was added dropwise at 0° C. The mixture was stirred at 23° C. for 0.5 h. The reaction mixture was quenched by addition NaHCO₃5 mL, and then diluted with H₂O 5 mL and extracted with DCM (10 mL×3). The combined organic layers were washed with brine (5 mL×2), dried over Na₂SO₄, filtered and concentrated under reduced pressure to give a residue. The residue was purified by prep-HPLC (column: Phenomenex C18 150*25 mm*10 μm; mobile phase: [water (NH₄HCO₃)-ACN]; B %: 44%-74%, 5 min) to afford N-[3-[6-amino-5-[2-[[(E)-4-[3-[4-[3-[5-[[[3-ethyl-5-[(2S)-2-(2-hydroxyethyl)-1-piperidyl]pyrazolo[1,5-a]pyrimidin-7-yl]amino methyl]-2-pyridyl]oxy]propyl]piperazin-1-yl]propyl-methyl-amino]but-2-enoyl]-methyl-amino]ethoxy]pyrimidin-4-yl]-5-fluoro-2-methyl-phenyl]-4-cyclopropyl-2-fluoro-benzamide (11 mg, 9 μmol, 24% yield, 94% purity) as a white solid. ¹H NMR: (400 MHz, MeOD) δ 8.28-8.12 (m, 2H), 7.89-7.60 (m, 4H), 7.12-6.94 (m, 3H), 6.79-6.77 (m, 1H), 6.78-6.67 (m, 1H), 6.66-6.42 (m, 1H), 5.52 (s, 1H), 4.80-4.73 (m, 1H), 4.53 (s, 2H), 4.30 (t, J=6.4 Hz, 2H), 4.02 (d, J=11.6 Hz, 1H), 3.73-3.51 (m, 5H), 3.42-3.35 (m, 1H), 3.22-3.14 (m, 2H), 2.99 (t, J=12.4 Hz, 1H), 2.88 (s, 2H), 2.64-2.47 (m, 10H), 2.42-2.34 (m, 4H), 2.33-2.27 (m, 2H), 2.24 (d, J=9.2 Hz, 3H), 2.17-1.89 (m, 8H), 1.76-1.61 (m, 8H), 1.55-1.51 (m, 1H), 1.23 (t, J=7.2 Hz, 3H), 1.14-1.04 (m, 2H), 0.85-0.77 (m, 2H). LC-MS: MS (ES⁺): RT=2.149 min, m/z=557.6 [1/2M+H⁺], LCMS method 05.

### Example 61—the Synthetic Route for I-82

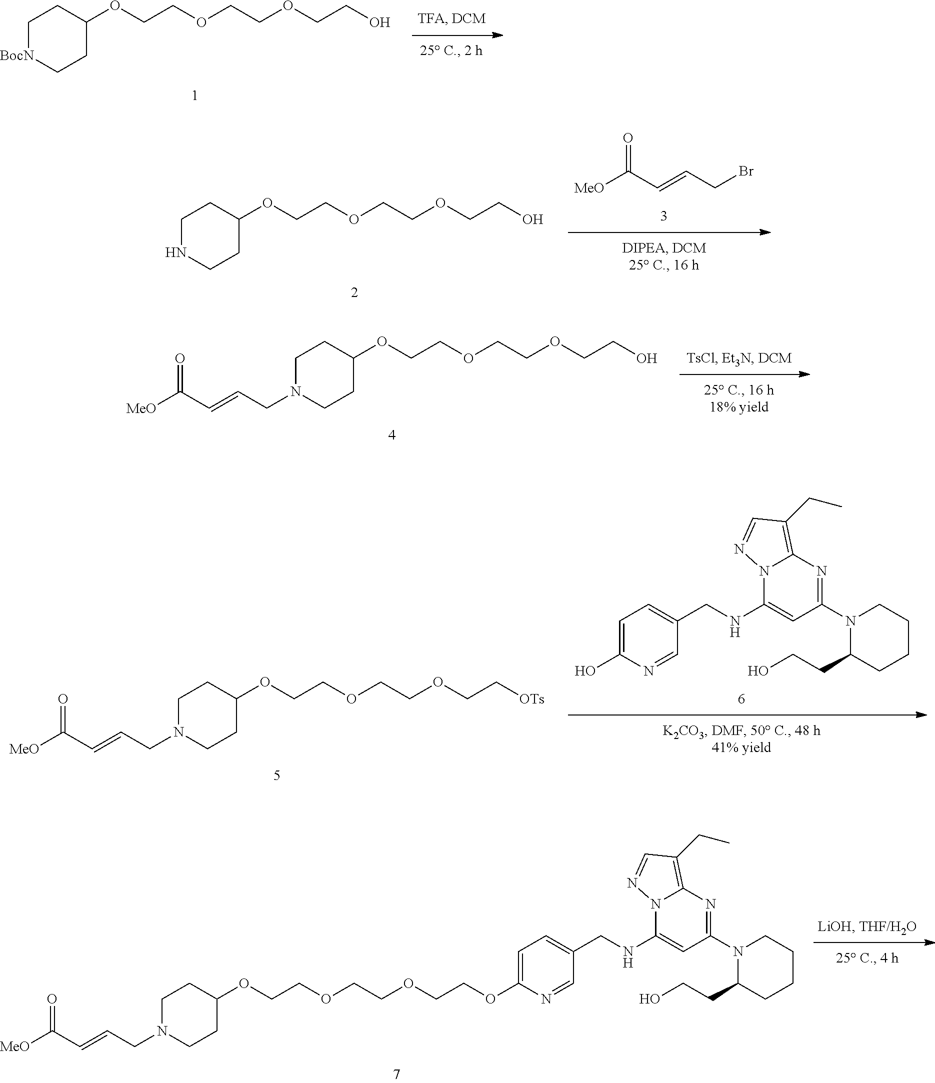

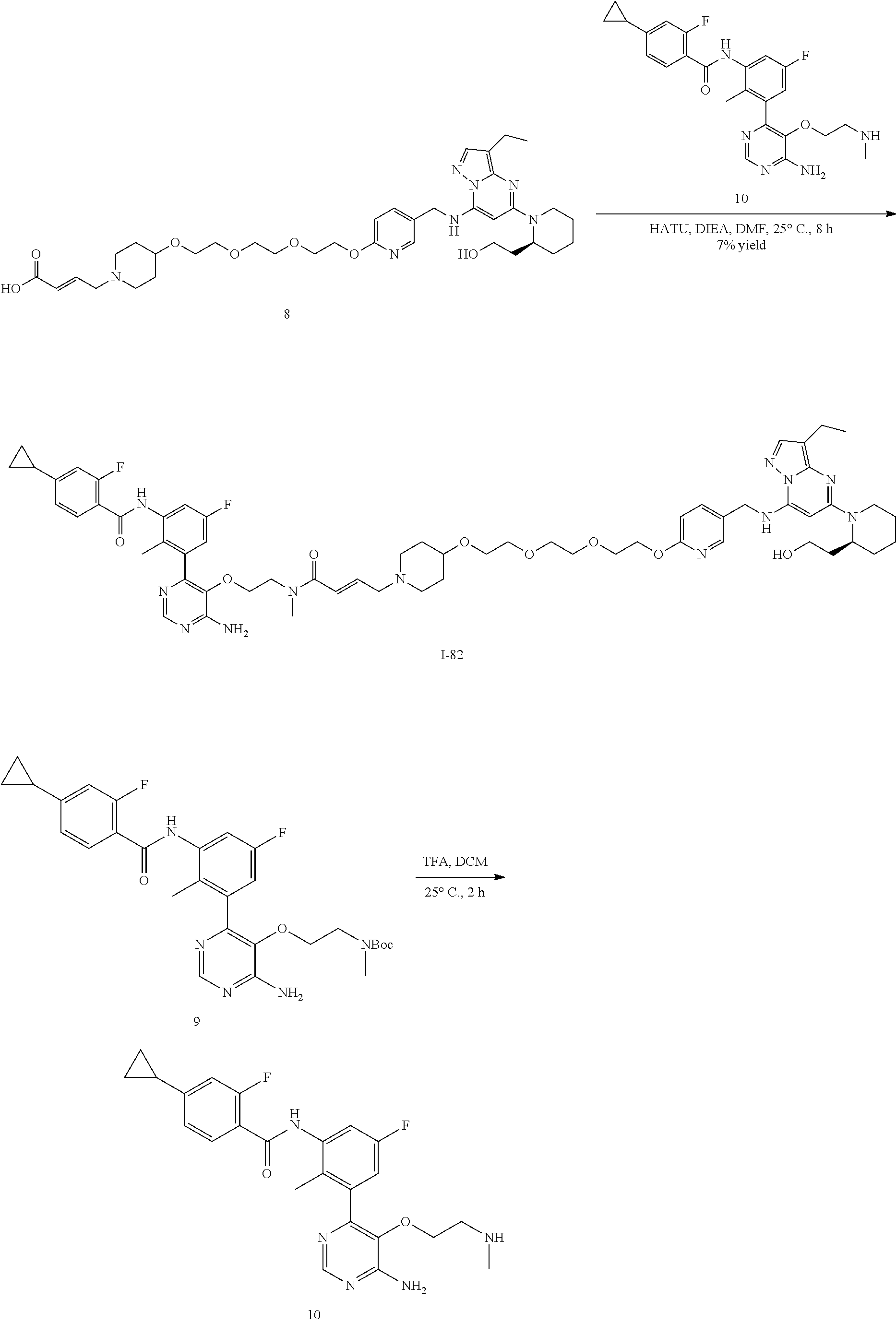

General Information: Synthetic route for compound 9 is described in US2015/152068.

Step 1. To a solution of tert-butyl 4-(2-(2-(2-hydroxyethoxy)ethoxy)ethoxy) piperidine-1-carboxylate (4.5 g, 13.5 mmol, 1.0 equiv) in DCM (20 mL) was added TFA (13.9 g, 121 mmol, 9.0 mL, 9.0 equiv). The mixture was stirred at 25° C. for 2 h. The reaction mixture was concentrated under reduced pressure to give a residue was used into the next step without further purification. Compound 2-(2-(2-(piperidin-4-yloxy)ethoxy)ethoxy) ethanol (4.5 g, 12.9 mmol, TFA salt) was obtained as a light yellow oil. ¹H NMR: (400 MHZ, CDCl₃) δ=4.46-4.41 (d, J=4.8 Hz, 2H), 3.74-3.71 (m, 2H), 3.64-3.61 (m, 7H), 3.57-3.55 (m, 2H), 3.35-3.28 (m, 2H), 3.16-3.12 (m, 2H), 1.94-1.90 (m, 4H).

Step 2. To a solution of 2-(2-(2-(piperidin-4-yloxy)ethoxy)ethoxy) ethanol (1.0 g, 2.9 mmol, 1.0 equiv, TFA, salt) in DCM (10 mL) was added TEA (874 mg, 8.6 mmol, 1.2 mL, 3.0 equiv) and (E)-methyl 4-bromobut-2-enoate (515 mg, 2.8 mmol, 339 μL, 1.0 equiv). The mixture was stirred at 25° C. for 16 h. The reaction mixture was diluted with H₂O (50 mL) and extracted with EA (40 mL×3). The combined organic layers were washed with water (50 mL×2), dried over Na₂SO₄, filtered and concentrated under reduced pressure to give a residue. The residue was purified by column chromatography (SiO₂, DCM: MeOH=20/1 to 10/1). Compound (E)-methyl 4-(4-(2-(2-(2-hydroxyethoxy)ethoxy)ethoxy) piperidin-1-yl) but-2-enoate (550 mg, 1.7 mmol) was obtained as a light yellow oil. ¹H NMR: (400 MHZ, CDCl₃) δ=6.89 (t, J=15.6, 6.2 Hz, 1H), 5.95-5.85 (d, J=15.6 Hz, 1H), 3.67 (s, 3H), 3.66 (s, 2H), 3.61 (d, J=0.8 Hz, 4H), 3.56-3.55 (m, 4H), 3.32-3.26 (m, 1H), 3.08-3.03 (d, J=6.0 Hz, 2H), 2.71-2.65 (m, 2H), 2.15-2.09 (m, 2H), 1.81-1.80 (m, 1H), 1.82-1.78 (m, 2H), 1.72-1.67 (m, 2H), 1.62-1.55 (m, 2H).

Step 3. To a solution of (E)-methyl 4-(4-(2-(2-(2-hydroxyethoxy)ethoxy)ethoxy) piperidin-1-yl) but-2-enoate (600 mg, 1.7 mmol, 1.0 equiv) in DCM (10.0 mL) was added TEA (504 mg, 5.0 mmol, 693 μL, 3.0 equiv) and TsCl (234 mg, 3.3 mmol, 2.0 equiv). The mixture was stirred at 25° C. for 16 h. The reaction mixture was concentrated under reduced pressure to remove solvent. The residue was diluted with H₂O (50 mL) and extracted with EA (50 mL×3). The combined organic layers were washed with H₂O (50 mL), dried over Na₂SO₄ filtered and concentrated under reduced pressure to give a residue. The crude product was purified by reversed-phase HPLC, column: YMC Triart C18 250*50 mm*7 μm; mobile phase: [water (NH₄HCO₃)-ACN]; B %: 40%-70%, 8 min, Compound (E)-methyl 4-(4-(2-(2-(2-(tosyloxy)ethoxy)ethoxy)ethoxy) piperidin-1-yl) but-2-enoate (150 mg, 309 μmol, 18% yield) was obtained as a yellow oil. ¹H NMR: (400 MHZ, CDCl₃) δ=7.74-7.72 (d, J=8.0 Hz, 2H), 7.29-7.26 (d, J=8.0 Hz, 2H), 6.94-6.84 (m, 1H), 5.95-5.86 (d, J=15.6 Hz, 1H), 4.09 (d, J=16.6, 4.7 Hz, 2H), 3.67 (s, 3H), 3.64-3.61 (m, 4H), 3.59 (s, 2H), 3.56 (s, 3H), 3.53-3.53 (m, 2H), 3.31-3.25 (m, 1H), 3.09-3.03 (d, J=5.6 Hz, 2H), 2.71-2.62 (m, 2H), 2.38 (s, 3H), 2.14-2.05 (m, 2H), 1.78-1.81 (m, 2H), 1.67 (m, 2H), 1.60-1.58 (m, 2H).

Step 4. To a solution of (E)-methyl 4-(4-(2-(2-(2-(tosyloxy)ethoxy)ethoxy) ethoxy) piperidin-1-yl) but-2-enoate (100 mg, 206 μmol, 1.5 equiv) in DMF (1 mL) was added K₂CO₃ (56 mg, 412 μmol, 3.0 equiv) and(S)-5-(((3-ethyl-5-(2-(2-hydroxyethyl) piperidin-1-yl) pyrazolo[1,5-a]pyrimidin-7-yl)amino) methyl) pyridin-2-ol (54 mg, 137 μmol, 1.0 equiv). The mixture was stirred at 50° C. for 48 h. The reaction mixture was diluted with H₂O (50 mL) and extracted with EA (20 mL×3). The combined organic layers were washed with H₂O (50 mL), dried over Na₂SO₄, filtered and concentrated under reduced pressure to give a residue. The crude product was purified by reversed-phase HPLC (column: Waters xbridge 150*25 mm*10 μm; mobile phase: [water (NH₄HCO₃)-ACN]; B %: 36%-66%,8 min) to give (S,E)-methyl 4-(4-(2-(2-(2-((5-(((3-ethyl-5-(2-(2-hydroxyethyl) piperidin-1-yl) pyrazolo[1,5-a]pyrimidin-7-yl)amino) methyl) pyridin-2-yl)oxy)ethoxy)ethoxy)ethoxy) piperidin-1-yl) but-2-enoate (40 mg, 56 μmol, 41% yield) as a light yellow oil. ¹H NMR: (400 MHZ, CDCl₃)8=7.63 (s, 1H), 7.51 (s, 1H), 7.37 (d, J=8.0 Hz, 1H), 7.01-6.90 (m, 1H), 6.64-6.54 (m, 1H), 6.39-6.30 (m, 1H), 6.03-5.89 (m, 1H), 5.34-5.27 (m, 1H), 5.11-5.03 (m, 1H), 4.30-4.22 (m, 2H), 4.19-4.07 (m, 2H), 3.78 (s, 2H), 3.74 (s, 3H), 3.56 (d, J=3.6 Hz, 8H), 3.39-3.27 (m, 2H), 3.12-3.07 (m, 2H), 3.06-3.00 (m, 1H), 2.77-2.67 (m, 2H), 2.64-2.57 (m, 2H), 2.31-2.22 (m, 2H), 2.12 (s, 2H), 2.07-2.00 (m, 1H), 1.90-1.84 (m, 2H), 1.68 (m, 5H), 1.64-1.50 (m, 4H), 1.25 (t, J=6.8 Hz, 3H).

Step 5. To a solution of methyl (S,E)-methyl 4-(4-(2-(2-(2-((5-(((3-ethyl-5-(2-(2-hydroxyethyl) piperidin-1-yl) pyrazolo[1,5-a]pyrimidin-7-yl)amino) methyl) pyridin-2-yl)oxy)ethoxy)ethoxy)ethoxy) piperidin-1-yl) but-2-enoate (30 mg, 42 μmol, 1.0 equiv) in THF (1 mL) and H₂O (0.3 mL), was added LiOH·H₂O (5 mg, 127 μmol, 3.0 equiv). The mixture was stirred at 25° C. for 4 h. The reaction mixture was concentrated under reduced pressure to give a residue and used into the next step without further purification. Compound (S,E)-4-(4-(2-(2-(2-((5-(((3-ethyl-5-(2-(2-hydroxyethyl) piperidin-1-yl) pyrazolo[1,5-a]pyrimidin-7-yl)amino) methyl) pyridin-2-yl)oxy)ethoxy)ethoxy)ethoxy) piperidin-1-yl) but-2-enoic acid (29 mg, 41 μmol, 98% yield) was obtained as a yellow oil.

Step 6. To a solution of tert-butyl(2-((4-amino-6-(3-(4-cyclopropyl-2-fluoro benzamido)-5-fluoro-2-methylphenyl)pyrimidin-5-yl)oxy) ethyl)(methyl) carbamate (20 mg, 36 μmol, 1.0 equiv) in DCM (3 mL) was added TFA (1 mL, 14 mmol, 373 equiv). The mixture was stirred at 25° C. for 2 h. The reaction mixture was concentrated under reduced pressure to give a residue. Compound N-(3-(6-amino-5-(2-(methylamino)ethoxy) pyrimidin-4-yl)-5-fluoro-2-methylphenyl)-4-cyclopropyl-2-fluorobenzamide (20 mg, 35 μmol, 97% yield, TFA) was obtained as a yellow oil.

Step 7. To a solution of (S,E)-4-(4-(2-(2-(2-((5-(((3-ethyl-5-(2-(2-hydroxyethyl) piperidin-1-yl) pyrazolo[1,5-a]pyrimidin-7-yl)amino) methyl) pyridin-2-yl)oxy)ethoxy)ethoxy)ethoxy) piperidin-1-yl) but-2-enoic acid (19 mg, 41 μmol, 1.0 equiv) in DMF (1 mL) was added DIPEA (16 mg, 125 μmol, 21 μL, 3.0 equiv) and HATU (16 mg, 41 μmol, 1.0 equiv), N-(3-(6-amino-5-(2-(methylamino)ethoxy) pyrimidin-4-yl)-5-fluoro-2-methylphenyl)-4-cyclopropyl-2-fluorobenzamide (29 mg, 42 μmol, 1.0 equiv). The mixture was stirred at 25° C. for 8 h. The reaction mixture was concentrated under reduced pressure to give a residue. The crude product was purified by reversed-phase HPLC column: Waters xbridge 150*25 mm 10 μm; mobile phase: [water (NH₄HCO₃)-ACN]; B %: 40%-70%, 8 min. Compound (S,E)-N-(3-(6-amino-5-(2-(4-(4-(2-(2-(2-((5-(((3-ethyl-5-(2-(2-hydroxyethyl) piperidin-1-yl) pyrazolo[1,5-a]pyrimidin-7-yl)amino) methyl) pyridin-2-yl)oxy)ethoxy)ethoxy)ethoxy) piperidin-1-yl)-N-methylbut-2-enamido)ethoxy) pyrimidin-4-yl)-5-fluoro-2-methylphenyl)-4-cyclopropyl-2-fluorobenzamide (3 mg, 3.0 μmol, 7% yield) was obtained as a yellow solid. ¹H NMR: (400 MHZ, CD₃OD) δ=8.25-8.17 (m, 2H), 7.85 (d, J=8.2, 10.8 Hz, 1H), 7.79-7.72 (m, 1H), 7.71-7.63 (m, 2H), 7.09-6.97 (m, 3H), 6.84-6.79 (m, 1H), 6.79-6.66 (m, 1H), 6.64-6.40 (m, 1H), 5.54-5.50 (m, 1H), 4.57-4.53 (m, 2H), 4.45-4.40 (m, 2H), 4.07-4.00 (m, 1H), 3.87-3.82 (m, 2H), 3.70 (m, 6H), 3.62-3.54 (m, 6H), 3.33 (m, 5H), 3.15-2.96 (m, 3H), 2.88-2.86 (m, 1H), 2.74-2.66 (m, 2H), 2.60-2.57 (m, 2H), 2.56-2.52 (m, 1H), 2.13-2.10 (m, 3H), 2.06-2.05 (m, 7H), 1.90-1.82 (m, 2H), 1.74-1.65 (m, 4H), 1.61-1.53 (m, 2H), 1.27-1.21 (m, 3H), 1.13-1.08 (m, 2H), 0.83-0.77 (m, 2H). LC-MS: MS (ES⁺): RT=2.357 min, m/z=1132.5 [M+H⁺], LCMS method 05.

### Example 62—the Synthetic Route for I-83

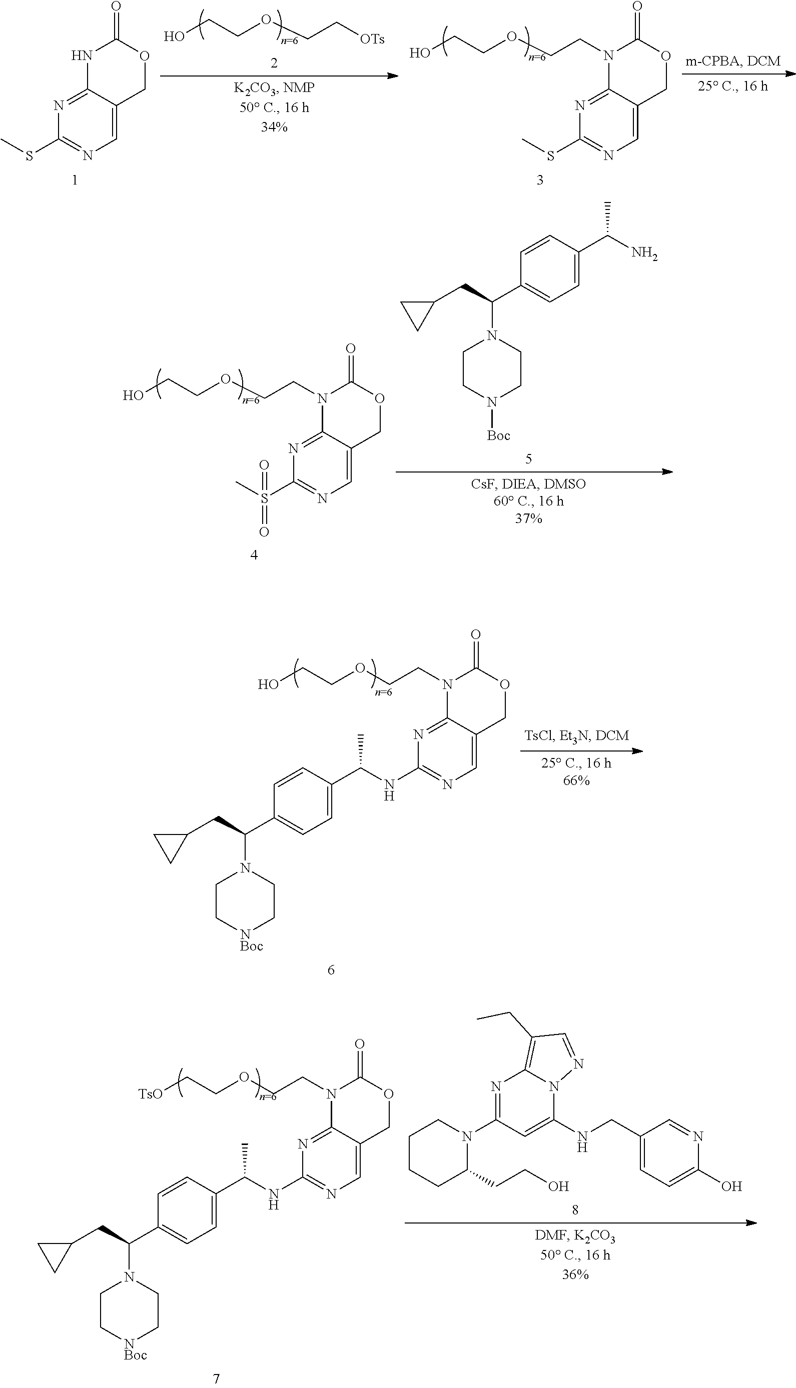

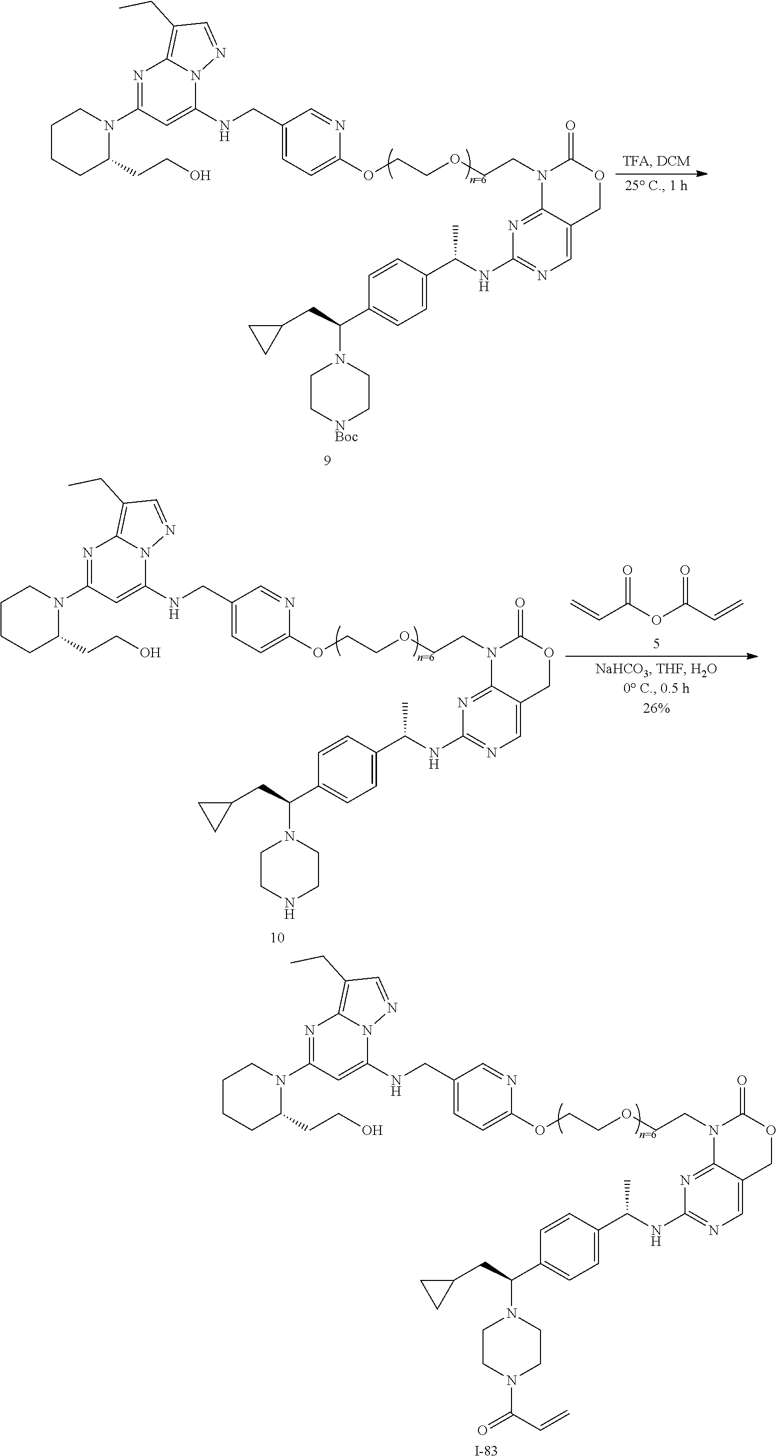

General Information: Compound 1 is described in WO2017/19429. Compound 2 is described in WO2021/102052. Compound 5 is a described in WO2018/111707.

Step 1. To a mixture of 7-methylsulfanyl-1,4-dihydropyrimido[4,5-d][1,3]oxazin-2-one (1.7 g, 8.62 mmol, 1.0 equiv) and 2-[2-[2-[2-[2-[2-(2-hydroxyethoxy)ethoxy]ethoxy]ethoxy]ethoxy]ethoxy]ethyl 4-methylbenzenesulfonate (4.14 g, 8.62 mmol, 1.0 equiv) in NMP (20 mL) was added K₂CO₃ (2.38 g, 17.2 mmol, 2.0 equiv), the mixture was stirred at 50° C. for 16 h. The mixture was filtered and concentrated. The residue was purified by prep-HPLC (column: Phenomenex luna C18 (250*70 mm*10 μm); mobile phase: [water (FA)-ACN]; B %: 25%-50%,20 min) to give 1-[2-[2-[2-[2-[2-[2-(2-hydroxyethoxy)ethoxy]ethoxy]ethoxy]ethoxy]ethoxy]ethyl]-7-methylsulfanyl-4H-pyrimido[4,5-d][1,3]oxazin-2-one (1.5 g, 34% yield).

Step 2. To a solution of 1-[2-[2-[2-[2-[2-[2-(2-hydroxyethoxy)ethoxy]ethoxy]ethoxy]ethoxy]ethoxy]ethyl]-7-methylsulfanyl-4H-pyrimido[4,5-d][1,3]oxazin-2-one (340 mg, 672 μmol, 1.0 equiv) in DCM (5 mL) was added m-CPBA (341 mg, 1.68 mmol, 85% purity, 2.5 equiv), the mixture was stirred at 25° C. for 16 h. Et₃N(0.2 mL) was added to the mixture solution and stirred for 0.5 h. The solution was concentrated to give crude 1-[2-[2-[2-[2-[2-[2-(2-hydroxyethoxy)ethoxy]ethoxy]ethoxy]ethoxy]ethoxy]ethyl]-7-methylsulfonyl-4H-pyrimido[4,5-d][1,3]oxazin-2-one (360 mg, crude) which was used into the next step without further purification.

Step 3. To a solution of 1-[2-[2-[2-[2-[2-[2-(2-hydroxyethoxy)ethoxy]ethoxy]ethoxy]ethoxy]ethoxy]ethyl]-7-methylsulfonyl-4H-pyrimido[4,5-d][1,3]oxazin-2-one (360 mg, 669 μmol, 1.0 equiv), tert-butyl 4-[(1S)-1-[4-[(1S)-1-aminoethyl]phenyl]-2-cyclopropyl-ethyl]piperazine-1-carboxylate (250 mg, 669 μmol, 1.0 equiv) and CsF (305 mg, 2.01 mmol, 3.0 equiv) in DMSO (2 mL) was added DIEA (129 mg, 1.0 mmol, 1.5 equiv), the mixture was stirred at 60° C. for 16 h. The mixture was filtered and concentrated. The residue was purified by prep-HPLC (column: Phenomenex luna C18 150*40 mm*15 μm; mobile phase: [water (FA)-ACN]; B %: 13%-43%, 10 min) to give tert-butyl 4-[(1S)-2-cyclopropyl-1-[4-[(1S)-1-[[1-[2-[2-[2-[2-[2-[2-(2-hydroxyethoxy)ethoxy]ethoxy]ethoxy]ethoxy]ethoxy]ethyl]-2-oxo-4H-pyrimido[4,5-d][1,3]oxazin-7-yl]amino]ethyl]phenyl]ethyl]piperazine-1-carboxylate (210 mg, 37% yield).

Step 4. To a solution of tert-butyl 4-[(1S)-2-cyclopropyl-1-[4-[(1S)-1-[1-[2-[2-[2-[2-[2-[2-(2-hydroxyethoxy)ethoxy]ethoxy]ethoxy]ethoxy]ethoxy]ethyl]-2-oxo-4H-pyrimido[4,5-d][1,3]oxazin-7-yl]amino]ethyl]phenyl]ethyl]piperazine-1-carboxylate (210 mg, 252 μmol, 1.0 equiv) and TEA (204 mg, 2.02 mmol, 8.0 equiv) in DCM (3 mL) was added TosCl (240 mg, 1.26 mmol, 5.0 equiv) at 0° C., the mixture was stirred at 25° C. for 16 h. The mixture was concentrated and purified by prep-HPLC (column: Waters Xbridge 150*25 mm*5 μm; mobile phase: [water (NH₄HCO₃)-ACN]; B %: 57%-87%, 8 min) to give tert-butyl 4-[(1S)-2-cyclopropyl-1-[4-[(1S)-1-[2-oxo-1-[2-[2-[2-[2-[2-[2-[2-(p-tolylsulfonyloxy)ethoxy]ethoxy]ethoxy]ethoxy]ethoxy]ethoxy]ethyl]-4H-pyrimido[4,5-d][1,3]oxazin-7-yl]amino]ethyl]phenyl]ethyl]piperazine-1-carboxylate (165 mg, 66% yield).

Step 5. To a solution of tert-butyl 4-[(1S)-2-cyclopropyl-1-[4-[(1S)-1-[[2-oxo-1-[2-[2-[2-[2-[2-[2-[2-(p-tolylsulfonyloxy)ethoxy]ethoxy]ethoxy]ethoxy]ethoxy]ethoxy]ethyl]-4H-pyrimido[4,5-d][1,3]oxazin-7-yl]amino]ethyl]phenyl]ethyl]piperazine-1-carboxylate (165 mg, 167 μmol, 1.0 equiv) and 5-[[[3-ethyl-5-[(2S)-2-(2-hydroxyethyl)-1-piperidyl]pyrazolo[1,5-a]pyrimidin-7-yl]amino methyl]pyridin-2-ol (66 mg, 167 μmol, 1.0 equiv) in DMF (2 mL) was added K₂CO₃ (46 mg, 334 μmol, 2.0 equiv), the mixture was stirred at 50° C. for 16 h. The mixture was filtered and concentrated. The residue was purified by prep-HPLC (column: Unisil 3-100 C18 Ultra 150*50 mm*3 μm; mobile phase: [water (FA)-ACN]; B %: 15%-45%, 10 min) to give 4-[(1S)-2-cyclopropyl-1-[4-[(1S)-1-[[1-[2-[2-[2-[2-[2-[2-[2-[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]ethoxy]ethoxy]ethyl]-2-oxo-4H-pyrimido[4,5-d][1,3]oxazin-7-yl]amino]ethyl]phenyl]ethyl]piperazine-1-carboxylate (73 mg, 36% yield).

Step 6. To a solution of tert-butyl 4-[(1S)-2-cyclopropyl-1-[4-[(1S)-1-[1-[2-[2-[2-[2-[2-[2-[2-[5-[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]ethoxy]ethoxy]ethyl]-2-oxo-4H-pyrimido[4,5-d][1,3]oxazin-7-yl]amino]ethyl]phenyl]ethyl]piperazine-1-carboxylate (73 mg, 60 μmol, 1.0 equiv) in DCM (1.5 mL) was added TFA (500 μL), the mixture was stirred at 25° C. for 1 h. The mixture was concentrated. The crude product 7-[[(1S)-1-[4-[(1S)-2-cyclopropyl-1-piperazin-1-yl-ethyl]phenyl]ethyl lamino]-1-[2-[2-[2-[2-[2-[2-[2-[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]ethoxy]ethoxy]ethyl]-4H-pyrimido[4,5-d][1,3]oxazin-2-one (79 mg, crude, TFA salt) was used into the next step without further purification.

Step 7. To a solution of 7-[[(1S)-1-[4-[(1S)-2-cyclopropyl-1-piperazin-1-yl-ethyl]phenyl]ethyl]amino]-1-[2-[2-[2-[2-[2-[2-[2-[5-[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]ethoxy]ethoxy]ethyl]-4H-pyrimido[4,5-d][1,3]oxazin-2-one (79 mg, 64 μmol, 1.0 equiv, TFA salt) and NaHCO₃ (1 M, 1.5 mL, 23.2 equiv) in THF (4.5 mL) was added prop-2-enoyl prop-2-enoate (8 mg, 64 μmol, 1.0 equiv) at 0° C., the mixture was stirred at 0° C. for 0.5 h. The mixture was diluted with water (20 mL) and extracted with EtOAc (20 mL*3). The combined organic layers were washed with brine (50 mL), dried over Na₂SO₄, filtered and concentrated. The residue was purified by prep-HPLC (column: Unisil 3-100 C18 Ultra 150*50 mm*3 μm; mobile phase: [water (FA)-ACN]; B %: 10%-40%, 10 min) to give 7-[[(1S)-1-[4-[(1S)-2-cyclopropyl-1-(4-prop-2-enoylpiperazin-1-yl) ethyl]phenyl]ethyl]amino]-1-[2-[2-[2-[2-[2-[2-[2-[[5-[[[3-ethyl-5-[(2S)-2-(2-hydroxyethyl)-1-piperidyl]pyrazolo[1,5-a]pyrimidin-7-yl]aminomethyl]-2-pyridyl]oxy]ethoxy]ethoxy]ethoxy]ethoxy]ethoxy]ethoxy]ethyl]-4H-pyrimido[4,5-d][1,3]oxazin-2-one (20 mg, 26% yield). ¹H NMR (400 MHZ, DMSO-d₆): δ 8.24-8.18 (m, 1H), 7.99 (s, 1H), 7.93-7.70 (m, 3H), 7.64 (s, 1H), 7.34-7.25 (m, 2H), 7.19-7.11 (m, 2H), 6.80-6.75 (m, 1H), 6.73-6.64 (m, 1H), 6.06-5.98 (m, 1H), 5.64-5.55 (m, 2H), 5.09 (s, 2H), 4.77-4.65 (m, 1H), 4.62-4.52 (m, 1H), 4.49-4.39 (m, 2H), 4.29-4.28 (m, 1H), 4.36-4.22 (m, 3H), 4.15-3.81 (m, 2H), 3.73-3.66 (m, 2H), 3.56-3.37 (m, 30H), 2.87-2.75 (m, 1H), 2.48-2.43 (m, 2H), 2.31-2.19 (m, 4H), 1.86-1.76 (m, 1H), 1.71-1.48 (m, 8H), 1.45-1.38 (m, 3H), 1.37-1.26 (m, 1H), 1.16 (t, J=7.6 Hz, 3H), 0.44-0.33 (m, 1H), 0.32-0.19 (m, 2H), 0.02--0.13 (m, 2H). LC-MS: MS (ES⁺): RT=1.875 min, m/z=1163.6 [M+H⁺]; LCMS method 25.

### Example 63—the Synthetic Route for I-84

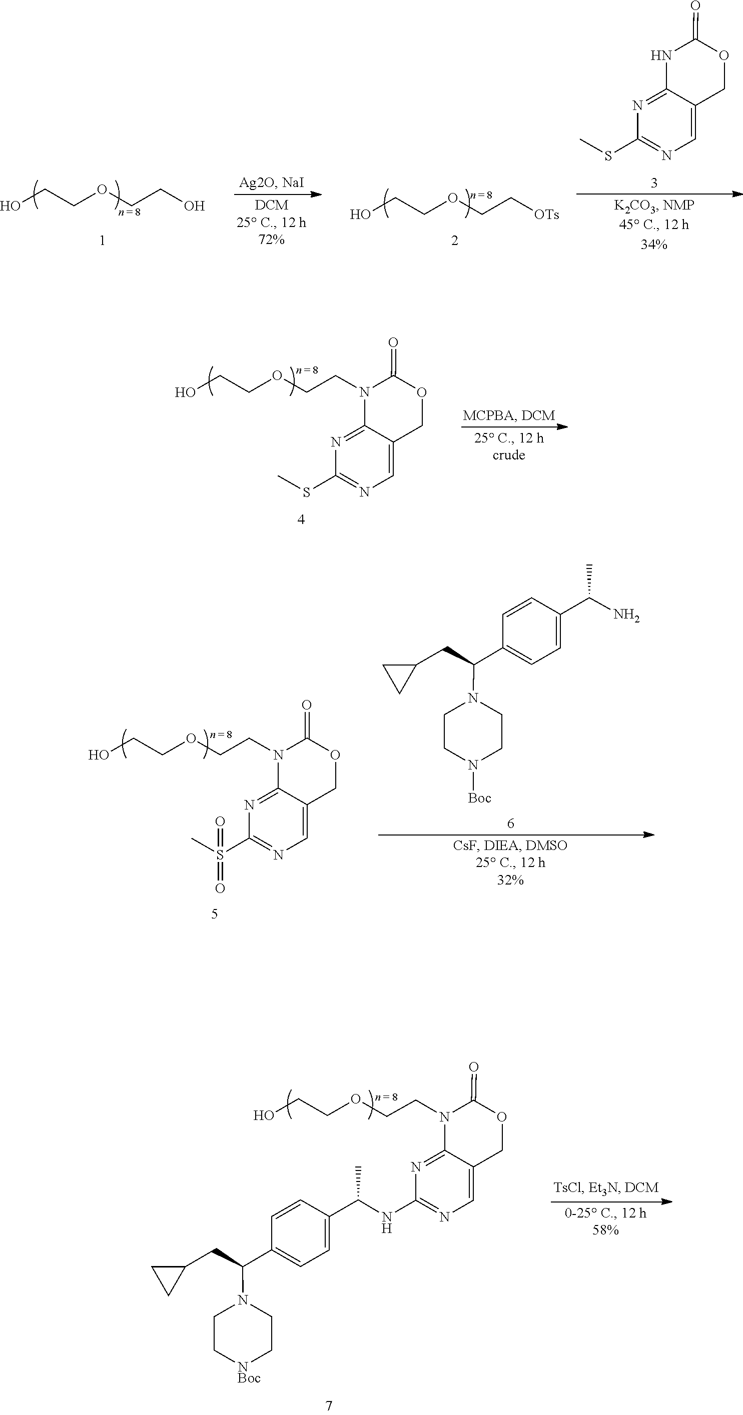

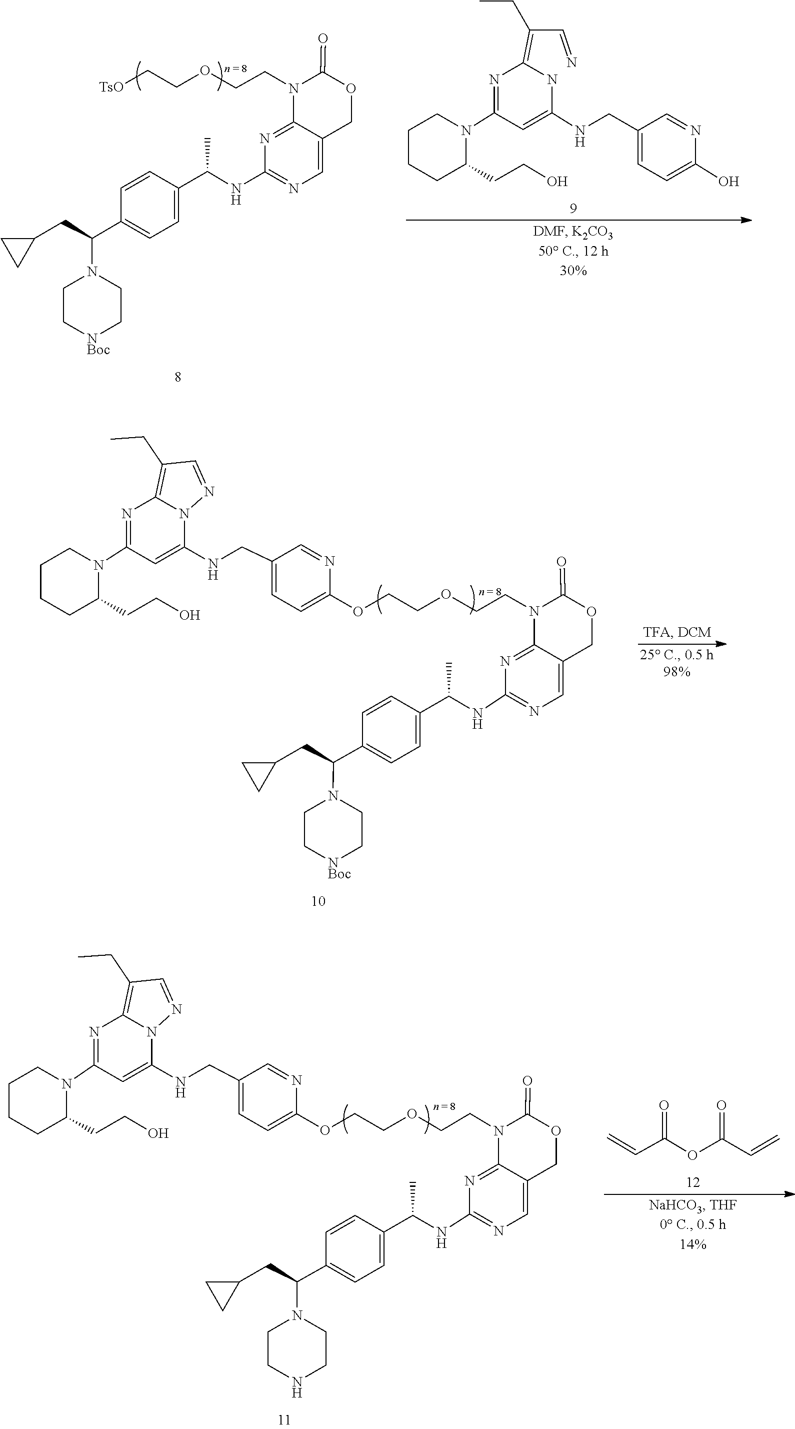

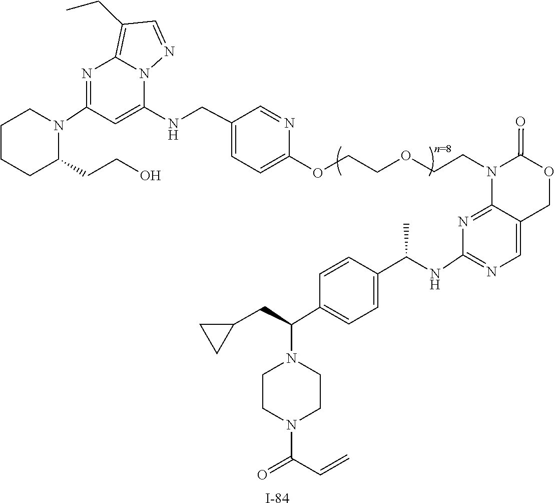

General Information: Compound 3 is described in CN113845528, 2021, A. Compound 6 is described in WO2018/111707, 2018, A¹. Compound 12 is described in U.S. Pat. No. 9,266,811.

Step 1. To a solution of 2-[2-[2-[2-[2-[2-[2-[2-(2-hydroxyethoxy)ethoxy]ethoxy]ethoxy]ethoxy]ethoxy]ethoxy]ethoxy]ethanol (10 g, 24 mmol, 1 equiv) in DCM (150 mL) was added Ag₂₀ (11 g, 48 mmol, 2 equiv) and NaI (3.9 g, 26 mmol, 1.1 equiv), and was added 4-methylbenzenesulfonyl chloride (4.6 g, 24 mmol, 1 equiv) at 0° C. The mixture was stirred at 25° C. for 12 h. The reaction mixture filtered and concentrated. The residue was purified by prep-HPLC (column: Kromasil Eternity XT 250*80 mm*10 μm; mobile phase: [water (NH₄HCO₃)-ACN]; B %: 25ACN %-55ACN %,18 min) to give compound 2-[2-[2-[2-[2-[2-[2-[2-(2-hydroxyethoxy)ethoxy]ethoxy]ethoxy]ethoxy]ethoxy]ethoxy]ethoxy]ethyl 4-methylbenzenesulfonate (10 g, 72% yield).

Step 2. To a solution of 2-[2-[2-[2-[2-[2-[2-[2-(2-hydroxyethoxy)ethoxy]ethoxy]ethoxy]ethoxy]ethoxy]ethoxy]ethoxy]ethyl 4-methylbenzenesulfonate (2 g, 3.52 mmol, 1.0 equiv) and 7-methylsulfanyl-1,4-dihydropyrimido[4,5-d][1,3]oxazin-2-one (693 mg, 3.52 mmol, 1.0 equiv) in NMP (20 mL) was added K₂CO₃ (972 mg, 7.03 mmol, 2.0 equiv). The mixture was stirred at 45° C. for 12 h. The reaction mixture filtered and concentrated. The residue was purified by prep-HPLC (column: Phenomenex luna C18 (250*70 mm, 10 μm); mobile phase: [water (FA)-ACN]; B %: 15%-40%, 20 min) to give compound 1-[2-[2-[2-[2-[2-[2-[2-[2-(2-hydroxyethoxy)ethoxy]ethoxy]ethoxy]ethoxy]ethoxy]ethoxy]ethoxy]ethyl]-7-methylsulfanyl-4H-pyrimido[4,5-d][1,3]oxazin-2-one (720 mg, 34% yield). ¹H NMR (400 MHZ, CDCl₃): δ 8.19 (s, 1H), 5.26 (s, 2H), 4.35 (t, J=5.9 Hz, 2H), 3.79 (t, J=5.9 Hz, 2H), 3.76-3.72 (m, 2H), 3.70-3.59 (m, 31H), 2.57 (s, 3H).

Step 3. To a solution of 1-[2-[2-[2-[2-[2-[2-[2-[2-(2-hydroxyethoxy)ethoxy]ethoxy]ethoxy]ethoxy]ethoxy]ethoxy]ethoxy]ethyl]-7-methylsulfanyl-4H-pyrimido[4,5-d][1,3]oxazin-2-one (620 mg, 1.04 mmol, 1 equiv) in DCM (10 mL) was added MCPBA (530 mg, 2.61 mmol, 85% purity, 2.5 equiv) at 0° C. The mixture was stirred at 25° C. for 12 h. The reaction mixture filtered and concentrated to give 1-[2-[2-[2-[2-[2-[2-[2-[2-(2-hydroxyethoxy)ethoxy]ethoxy]ethoxy]ethoxy]ethoxy]ethoxy]ethoxy]ethyl]-7-methylsulfonyl-4H-pyrimido[4,5-d][1,3]oxazin-2-one (653 mg, crude).

Step 4. To a solution of 1-[2-[2-[2-[2-[2-[2-[2-[2-(2-hydroxyethoxy)ethoxy]ethoxy]ethoxy]ethoxy]ethoxy]ethoxy]ethoxy]ethyl]-7-methylsulfonyl-4H-pyrimido[4,5-d][1,3]oxazin-2-one (418 mg, 669 μmol, 1 equiv) and tert-butyl 4-[(1S)-1-[4-[(1S)-1-aminoethyl]phenyl]-2-cyclopropyl-ethyl]piperazine-1-carboxylate (250 mg, 669 μmol, 1 equiv) in DMSO (3 mL) was added CsF (305 mg, 2.0 mmol, 3 equiv) and DIEA (129 mg, 1 mmol, 1.5 equiv). The mixture was stirred at 25° C. for 12 h. The mixture filtered and concentrated. The residue was purified by prep-HPLC (column: Phenomenex Synergi C18 150*25 mm*10 μm; mobile phase: [water (FA)-ACN]; B %: 14%-41%,9 min) to give compound tert-butyl 4-[(1S)-2-cyclopropyl-1-[4-[(1S)-1-[[1-[2-[2-[2-[2-[2-[2-[2-[2-(2-hydroxyethoxy)ethoxy]ethoxy]ethoxy]ethoxy]ethoxy]ethoxy]ethoxy]ethyl]-2-oxo-4H-pyrimido[4,5-d][1,3]oxazin-7-yl]amino]ethyl]phenyl]ethyl]piperazine-1-carboxylate (200 mg, 32% yield).

Step 5. To a solution of tert-butyl 4-[(1S)-2-cyclopropyl-1-[4-[(1S)-1-[1-[2-[2-[2-[2-[2-[2-[2-[2-(2-hydroxyethoxy)ethoxy]ethoxy]ethoxy]ethoxy]ethoxy]ethoxy]ethoxy]ethyl]-2-oxo-4H-pyrimido[4,5-d][1,3]oxazin-7-yl]amino]ethyl]phenyl]ethyl]piperazine-1-carboxylate (280 mg, 304 μmol, 1 equiv) in DCM (3 mL) was added TEA (500 μL) and was added 4-methylbenzenesulfonyl chloride (290 mg, 1.52 mmol, 5 equiv) at 0° C. The mixture was stirred at 25° C. for 12 h. The mixture filtered and concentrated. The residue was purified by prep-HPLC (column: Waters Xbridge C18 150*50 μm*10; mobile phase: [water (NH₄HCO₃)-ACN];

B %: 55%-85%, 11 min) to give tert-butyl 4-[(1S)-2-cyclopropyl-1-[4-[(1S)-1-[2-oxo-1-[2-[2-[2-[2-[2-[2-[2-[2-[2-(p-tolylsulfonyloxy)ethoxy]ethoxy]ethoxy]ethoxy]ethoxy]ethoxy]ethoxy]ethoxy]ethyl]-4H-pyrimido[4,5-d][1,3]oxazin-7-yl]amino]ethyl]phenyl]ethyl]piperazine-1-carboxylate (190 mg, 58% yield).

Step 6. To a solution of tert-butyl 4-[(1S)-2-cyclopropyl-1-[4-[(1S)-1-[2-oxo-1-[2-[2-[2-[2-[2-[2-[2-[2-[2-(p-tolylsulfonyloxy)ethoxy]ethoxy]ethoxy]ethoxy]ethoxy]ethoxy]ethoxy]ethoxy]ethyl]-4H-pyrimido[4,5-d][1,3]oxazin-7-yl]amino]ethyl]phenyl]ethyl]piperazine-1-carboxylate (190 mg, 177 μmol, 1 equiv) and 5-[[[3-ethyl-5-[(2S)-2-(2-hydroxyethyl)-1-piperidyl]pyrazolo[1,5-a]pyrimidin-7-yl]amino]methyl]pyridin-2-ol (70 mg, 177 μmol, 1 equiv) in DMF (2 mL) was added K₂CO₃ (48 mg, 354 μmol, 2 equiv). The mixture was stirred at 50° C. for 12 h. The reaction mixture filtered and concentrated under reduced pressure to give a residue. The residue was purified by prep-HPLC (column: Unisil 3-100 C18 Ultra 150*50 mm*3 μm; mobile phase: [water (FA)-ACN]; B %: 15%-45%, 10 min) to give compound tert-butyl 4-[(1S)-2-cyclopropyl-1-[4-[(1S)-1-[[1-[2-[2-[2-[2-[2-[2-[2-[2-[2-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]ethoxy]ethoxy]ethoxy]ethoxy]ethyl]-2-oxo-4H-pyrimido[4,5-d][1,3]oxazin-7-yl]amino]ethyl]phenyl]ethyl]piperazine-1-carboxylate (70 mg, 30% yield).

Step 7. To a solution of tert-butyl 4-[(1S)-2-cyclopropyl-1-[4-[(1S)-1-[1-[2-[2-[2-[2-[2-[2-[2-[2-[2-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]ethoxy]ethoxy]ethoxy]ethoxy]ethyl]-2-oxo-4H-pyrimido[4,5-d][1,3]oxazin-7-yl]amino]ethyl]phenyl]ethyl]piperazine-1-carboxylate (70 mg, 53 μmol, 1 equiv) in DCM (3 mL) was added TFA (400 μL). The mixture was stirred at 25° C. for 0.5 h. The mixture filtered and concentrated. The residue was used for next step directly to give compound 7-[[(1S)-1-[4-[(1S)-2-cyclopropyl-1-piperazin-1-yl-ethyl]phenyl]ethyl]amino]-1-[2-[2-[2-[2-[2-[2-[2-[2-[2-[5-[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]ethoxy]ethoxy]ethoxy]ethoxy]ethyl]-4H-pyrimido[4,5-d][1,3]oxazin-2-one (70 mg, 98% yield).

Step 8. To a solution ofprop-2-enoyl prop-2-enoate (6 mg, 53 μmol, 1 equiv) in THF (2 mL) was added NaHCO₃ (1 M, 1.39 mL, 26 equiv) and 7-[[(1S)-1-[4-[(1S)-2-cyclopropyl-1-piperazin-1-yl-ethyl]phenyl]ethyl]amino]-1-[2-[2-[2-[2-[2-[2-[2-[2-[2-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]ethoxy]ethoxy]ethoxy]ethoxy]ethyl]-4H-pyrimido[4,5-d][1,3]oxazin-2-one (70 mg, 53 μmol, 1 equiv, TFA salt). The mixture was stirred at 0° C. for 0.5 h. The reaction mixture was diluted with water (5 mL) and extracted with ethyl acetate (5 mL*3). The combined organic layers were washed with brine (10 mL), dried over sodium sulfate, filtered and concentrated. The residue was purified by prep-HPLC (column: Unisil 3-100 C18 Ultra 150*50 mm*3 μm; mobile phase: [water (FA)-ACN]; B %: 10%-40%, 10 min) to give compound 7-[(1S)-1-[4-[(1S)-2-cyclopropyl-1-(4-prop-2-enoylpiperazin-1-yl) ethyl]phenyl]ethyl]amino]-1-[2-[2-[2-[2-[2-[2-[2-[2-[2-[5-[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]ethoxy]ethoxy]ethoxy]ethoxy]ethyl]-4H-pyrimido[4,5-d][1,3]oxazin-2-one (9 mg, 14% yield). ¹H NMR (400 MHZ, DMSO): δ 8.40 (s, 1H), 8.23 (d, J=2.1 Hz, 1H), 7.99 (s, 1H), 7.92-7.74 (m, 3H), 7.65 (s, 1H), 7.31-7.29 (m, 2H), 7.17 (d, J=7.8 Hz, 2H), 6.79 (d, J=8.7 Hz, 1H), 6.75-6.60 (m, 1H), 6.05-6.00 (m, 1H), 5.66-5.56 (m, 2H), 5.09 (s, 2H), 4.74-4.60 (m, 1H), 4.64-4.42 (m, 3H), 4.39-4.19 (m, 3H), 4.14-3.85 (m, 1H), 3.75-3.67 (m, 2H), 3.59-3.52 (m, 6H), 3.51-3.45 (m, 35H), 2.89-2.77 (m, 1H), 1.78-1.50 (m, 10H), 1.48-1.31 (m, 5H), 1.16 (t, J=7.5 Hz, 4H), 0.28-0.25 (m, 3H), 0.02-0.10 (m, 2H). LC-MS: MS (ES⁺): RT=1.794 min, m/z=626.4 [M+H⁺]; LCMS method 25.

### Example 64—the Synthetic Route for I-85

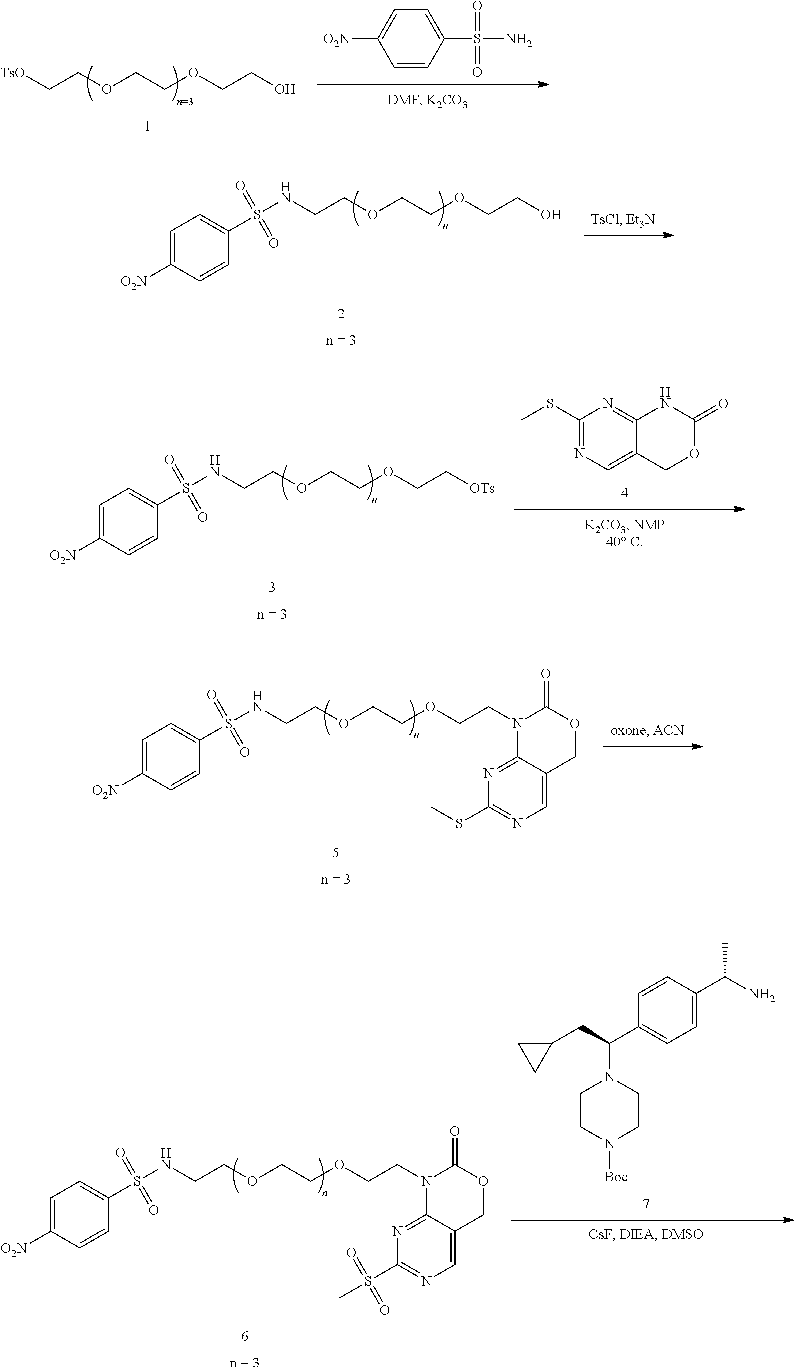

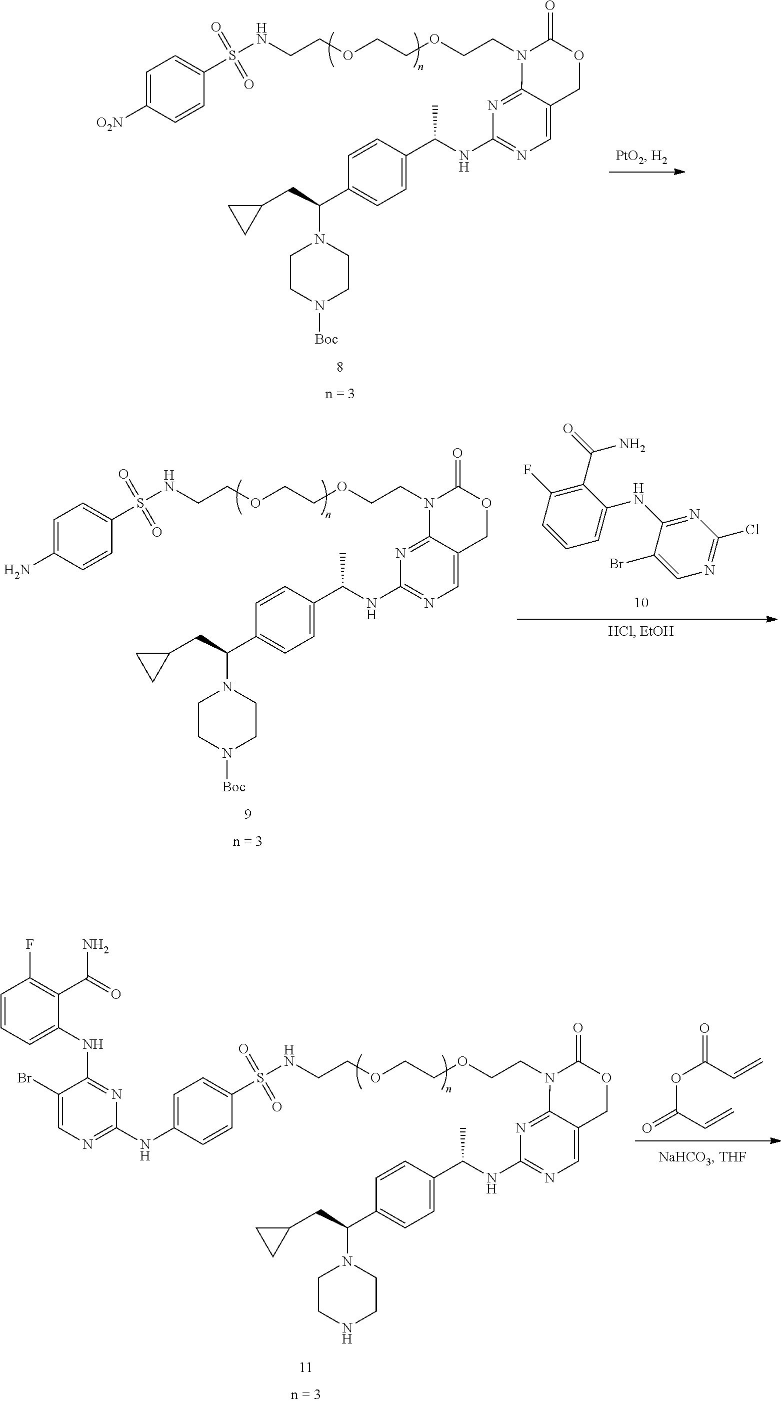

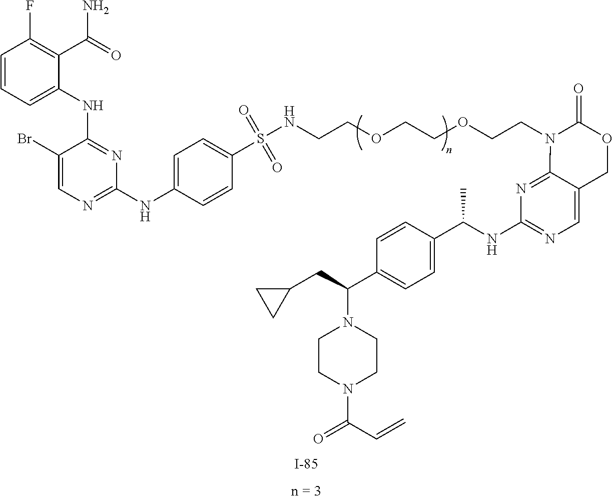

General Information: Compound 7 is described in WO2018/111707, 2018, A1.

Step 1. To a solution of 2-[2-[2-[2-(2-hydroxyethoxy)ethoxy]ethoxy]ethoxy]ethyl 4-methylbenzenesulfonate (6 g, 15.3 mmol, 1 eq) and 4-nitrobenzenesulfonamide (3.1 g, 15.3 mmol, 1 eq) in DMF (20 mL) was added K₂CO₃ (4.23 g, 30.6 mmol, 2 eq). The mixture was stirred at 70° C. for 12 h. The mixture was diluted with water (30 mL) and extracted with EtOAc (30 mL*3). The combined organic layers were washed with brine, dried over Na₂SO₄, filtered and concentrated. The residue was purified by column chromatography (SiO2,

Petroleum ether/Ethyl acetate=10/1 to 1/1) to give N-[2-[2-[2-[2-(2-hydroxyethoxy)ethoxy]ethoxy]ethoxy]ethyl]-4-nitro-benzenesulfonamide (2.1 g, 33% yield). ¹H NMR (400 MHZ, MeOD): δ 8.42 (dd, J=2.4, 8.8 Hz, 2H), 8.11 (dd, J=2.4, 8.8 Hz, 2H), 3.66-3.64 (m, 10H), 3.63-3.62 (m, 4H), 3.56-3.50 (m, 4H), 3.15-3.13 (m, 2H).

Step 2. To a solution of TosCl (1.42 g, 7.46 mmol, 1.5 eq) in DCM (20 mL) was added TEA (1.51 g, 14.9 mmol, 3 eq) and N-[2-[2-[2-[2-(2-hydroxyethoxy)ethoxy]ethoxy]ethoxy]ethyl]-4-nitro-benzenesulfonamide (2.1 g, 4.97 mmol, 1 eq) was added at 0° C. The mixture was stirred at 20° C. for 12 h and concentrated. The residue was purified by prep-HPLC (column: Waters Xbridge C18 150*50 mm*10 μm; mobile phase: [water (NH₄HCO₃)-ACN]; B %: 37%-67%,11 min,column: Waters Xbridge C18 150*50 mm*10 μm; mobile phase: [water (NH₄HCO₃)-ACN]; B %: 37%-67%,11 min) to give 2-[2-[2-[2-[2-[(4-nitrophenyl) sulfonylamino]ethoxy]ethoxy]ethoxy]ethoxy]ethyl 4-methylbenzenesulfonate (1.2 g,42% yield). ¹H NMR (400 MHZ, MeOD): δ 8.41 (dd, J=2.4, 8.8 Hz, 2H), 8.11 (dd, J=2.4, 8.8 Hz, 2H), 7.79 (d, J=8.4 Hz, 2H), 7.44 (d, J=8.4 Hz, 2H), 4.15-4.12 (m, 2H), 3.66-3.64 (m, 2H), 3.63-3.55 (m, 10H), 3.55-3.45 (m, 4H), 3.15-3.13 (m, 2H), 2.45 (s, 3H).

Step 3. To a solution of 7-methylsulfanyl-1,4-dihydropyrimido[4,5-d][1,3]oxazin-2-one (493 mg, 2.5 mmol, 1.5 eq) and 2-[2-[2-[2-[2-[(4-nitrophenyl)sulfonylamino]ethoxy]ethoxy]ethoxy]ethoxy]ethyl 4-methylbenzenesulfonate (960 mg, 1.66 mmol, 1 eq) in NMP (5 mL) was added K₂CO₃ (460 mg, 3.33 mmol, 2 eq). The mixture was stirred at 40° C. for 12 h. The mixture was concentrated and purified by prep-HPLC (column: Phenomenex luna C18 150*40 mm*15 μm; mobile phase: [water (FA)-ACN]; B %: 33%-63%, 10 min) to give N-(3-methyl-2-oxo-1,4-dihydroquinazolin-6-yl)-2-(4-piperidylmethoxy) benzenesulfonamide (45 mg, 97% yield).

Step 4. To a solution of N-[2-[2-[2-[2-[2-(7-methylsulfanyl-2-oxo-4H-pyrimido[4,5-d][1,3]oxazin-1-yl)ethoxy]ethoxy]ethoxy]ethoxy]ethyl]-4-nitro-benzenesulfonamide (500 mg, 831 μmol, 1 eq) in MeCN(4 mL) and H₂O (2 mL) was added Oxone (1.02 g, 1.66 mmol, 2 eq). The mixture was stirred at 25° C. for 12 h. The mixture was diluted with water (10 mL) and extracted with EtOAc (10 mL*3). The combined organic layers were washed with brine (10 mL), dried over Na₂SO₄, filtered and concentrated to give N-[2-[2-[2-[2-[2-(7-methylsulfonyl-2-oxo-4H-pyrimido[4,5-d][1,3]oxazin-1-yl)ethoxy]ethoxy]ethoxy]ethoxy]ethyl]-4-nitro-benzenesulfonamide (520 mg, 99% yield).

Step 5. To a solution of tert-butyl 4-[(1S)-1-[4-[(1S)-1-aminoethyl]phenyl]-2-cyclopropyl-ethyl]piperazine-1-carboxylate (277 mg, 742 μmol, 1 eq) and N-[2-[2-[2-[2-[2-(7-methylsulfonyl-2-oxo-4H-pyrimido[4,5-d][1,3]oxazin-1-yl)ethoxy]ethoxy]ethoxy]ethoxy]ethyl]-4-nitro-benzenesulfonamide (470 mg, 741.74 μmol, 1 eq) in DMSO (2 mL) was added CsF (338 mg, 2.23 mmol, 3 eq) and DIPEA (144 mg, 1.11 mmol, 1.5 eq). The mixture was stirred at 60° C. for 1 h and concentrated. The residue was purified by prep-HPLC (column: Phenomenex luna C18 150*40 mm*15 μm; mobile phase: [water (FA)-ACN]; B %: 20% -50% ,10 min) to give tert-butyl 4-[(1S)-2-cyclopropyl-1-[4-[(1S)-1-[[1-[2-[2-[2-[2-[2-[(4-nitrophenyl)sulfonylamino]ethoxy]ethoxy]ethoxy]ethoxy]ethyl]-2-oxo-4H-pyrimido[4,5-d][1,3]oxazin-7-yl]amino]ethyl]phenyl]ethyl]piperazine-1-carboxylate (170 mg, 25% yield). ¹H NMR (400 MHZ, MeOD): δ 8.41 (d, J=8.8 Hz, 2H), 8.10 (d, J=8.8 Hz, 2H), 7.35 (d, J=8.0 Hz, 2H), 7.25 (d, J=8.4 Hz, 2H), 4.58 (s, 6H), 3.62-3.30 (m, 24H), 3.15-3.13 (m, 2H), 2.45-2.25 (m, 2H), 1.92-1.90 (m, 1H), 1.57-1.50 (m, 4H), 1.41 (s, 9H), 0.40-0.20 (m, 3H).

Step 6. To a solution of tert-butyl 4-[(1S)-2-cyclopropyl-1-[4-[(1S)-1-[1-[2-[2-[2-[2-[2-[(4-nitrophenyl)sulfonylamino]ethoxy]ethoxy]ethoxy]ethoxy]ethyl]-2-oxo-4H-pyrimido[4,5-d][1,3]oxazin-7-yl]amino]ethyl]phenyl]ethyl]piperazine-1-carboxylate (170 mg, 183 μmol, 1 eq) in THF (5 mL) was added PtO2 (42 mg) under N₂ atmosphere. The mixture was stirred under H₂ (15 Psi.) at 25° C. for 1 h. The mixture was filtered and concentrated. The crude product was used directly for next step.

Step 7. To a solution of tert-butyl 4-[(1S)-1-[4-[(1S)-1-[[1-[2-[2-[2-[2-[2-[(4-aminophenyl)sulfonylamino]ethoxy]ethoxy]ethoxy]ethoxy]ethyl]-2-oxo-4H-pyrimido[4,5-d][1,3]oxazin-7-yl]amino]ethyl]phenyl]-2-cyclopropyl-ethyl]piperazine-1-carboxylate (110 mg, 122 μmol, 1 eq) and 2-[(5-bromo-2-chloropyrimidin-4-yl)amino]-6-fluoro-benzamide (43 mg, 122 μmol, 1 eq) in EtOH (1 mL) was added HCL (10 M, 1.23 μL, 0.1 eq) and H₂O (221 μg, 12 μmol, 0.1 eq). The mixture was stirred at 80° C. for 12 h. The mixture was concentrated and purified by prep-HPLC (column: Phenomenex luna C18 250*50 mm*15 μm; mobile phase: [water (FA)-ACN]; B %: 21%-51%, 10 min) to give 2-[[5-bromo-2-[4-[2-[2-[2-[2-[2-[7-[[(1S)-1-[4-[(1S)-2-cyclopropyl-1-piperazin-1-yl-ethyl]phenyl]ethyl]amino]-2-oxo-4H-pyrimido[4,5-d][1,3]oxazin-1-yl]ethoxy]ethoxy]ethoxy]ethoxy]ethylsulfamoyl]anilino]pyrimidin-4-yl]amino]-6-fluorobenzamide (40 mg, 29% yield).

Step 8. To a solution of prop-2-enoyl prop-2-enoate (2.2 mg, 17 μmol, 1 eq) in THF (0.5 mL) was added NaHCO₃ (1 mL). The mixture was stirred at 0° C. for 0.5 h. Then 2-[[5-bromo-2-[4-[2-[2-[2-[2-[2-[7-[[(1S)-1-[4-[(1S)-2-cyclopropyl-1-piperazin-1-y]ethyl]phenyl]ethyl]amino]-2-oxo-4H-pyrimido[4,5-d][1,3]oxazin-1-yl]ethoxy]ethoxy]ethoxy]ethoxy]ethylsulfamoyl]anilino]pyrimidin-4-yl]amino]-6-fluoro-benzamide (20 mg, 17 μmol, 1 eq, FA salt) was added at 0° C., the mixture was stirred at 0° C. for 0.5 h. The mixture was concentrated and purified by prep-HPLC (column: Phenomenex luna C18 250*50 mm*15 μm; mobile phase: [water (FA)-ACN]; B %: 23%-56%,10 min) to give 2-[[5-bromo-2-[4-[2-[2-[2-[2-[2-[7-[(1S)-1-[4-[(1S)-2-cyclopropyl-1-(4-prop-2-enoylpiperazin-1-yl) ethyl]phenyl]ethyl]amino]-2-oxo-4H-pyrimido[4,5-d][1,3]oxazin-1-yl]ethoxy]ethoxy]ethoxy]ethoxy]ethylsulfamoyl]anilino]pyrimidin-4-yl]amino]-6-fluoro-benzamide (3 mg, 14% yield). ¹H NMR (400 MHZ, MeOD): δ 8.50-8.38 (m, 2H), 8.24 (s, 1H), 7.85-7.82 (m, 2H), 7.72 (d, J=8.8 Hz, 2H), 7.52-7.49 (m, 1H), 7.33-7.31 (m, 2H), 7.22-7.16 (m, 2H), 7.02-6.96 (m, 1H), 6.66-6.61 (m, 1H), 6.15-6.10 (m, 1H), 5.69-5.66 (m, 1H), 3.63-3.59 (m, 20H), 3.55-3.45 (m, 6H), 3.10-3.05 (m, 2H), 2.40-2.03 (m, 4H), 1.85-1.75 (m, 1H), 1.55-1.45 (m, 3H), 1.30-1.25 (m, 1H), 0.3-0.22 (m, 3H), 0.02-0.00 (m, 2H). LC-MS: MS (ES⁺): RT=1.903 min, m/z=1183.3 [M+Na+]; LCMS method 25.

### Example 65—the Synthetic Route for I-86

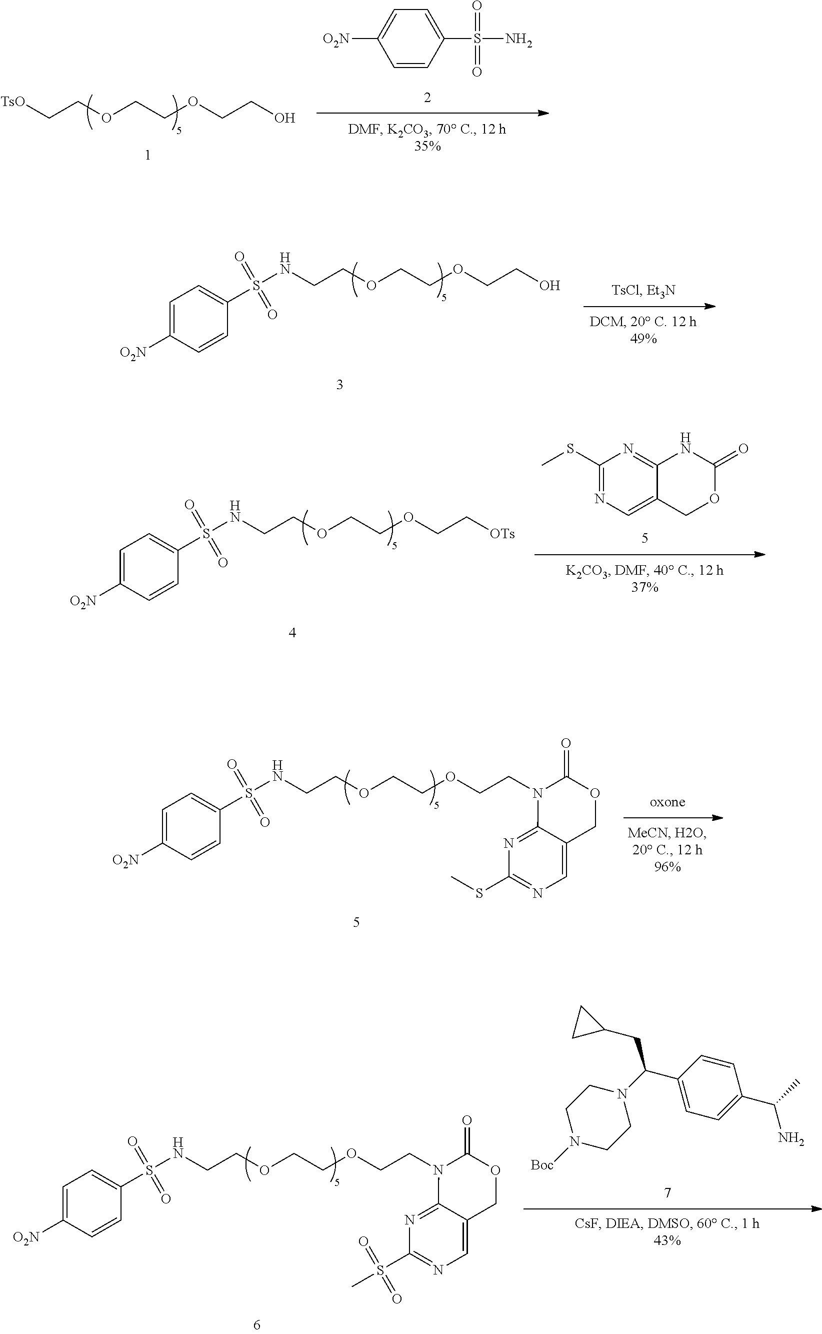

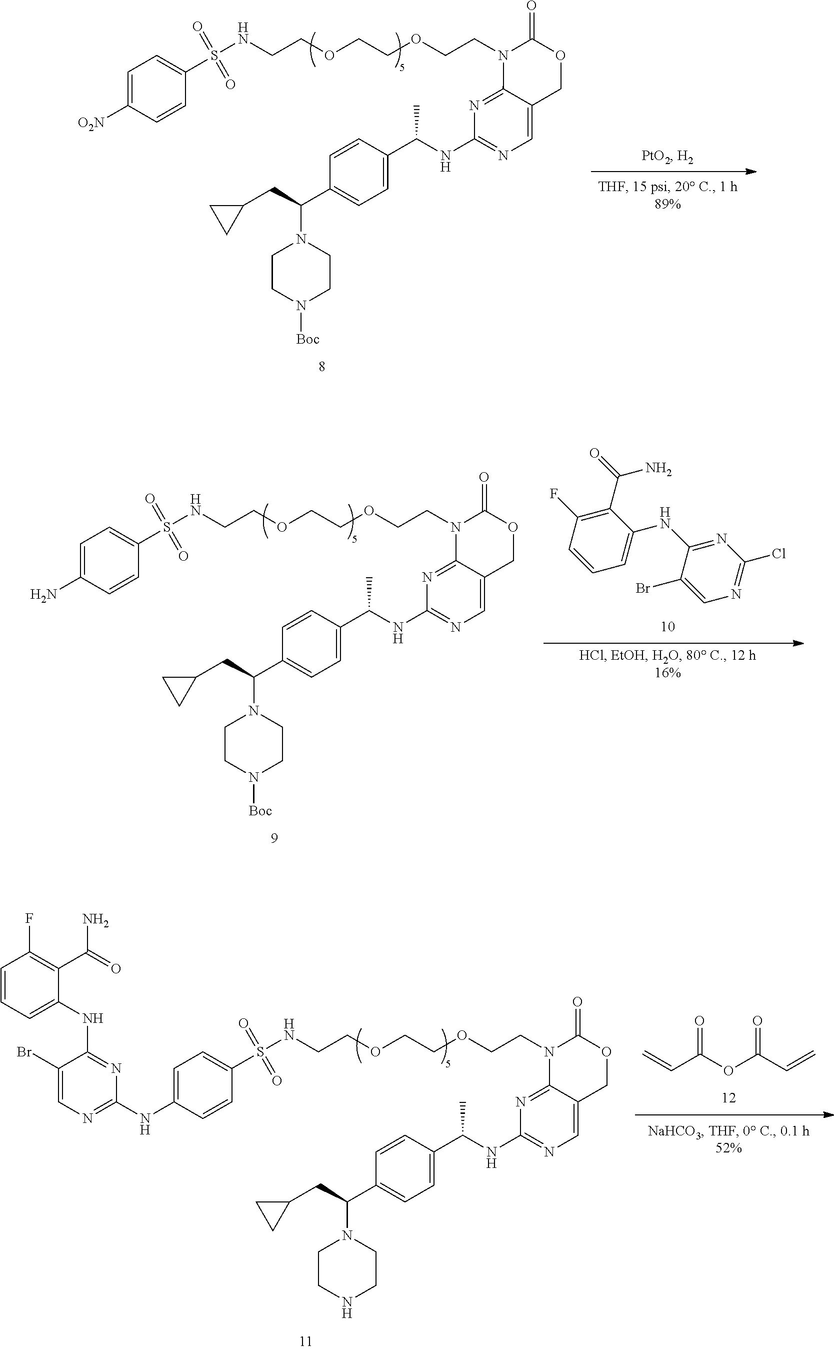

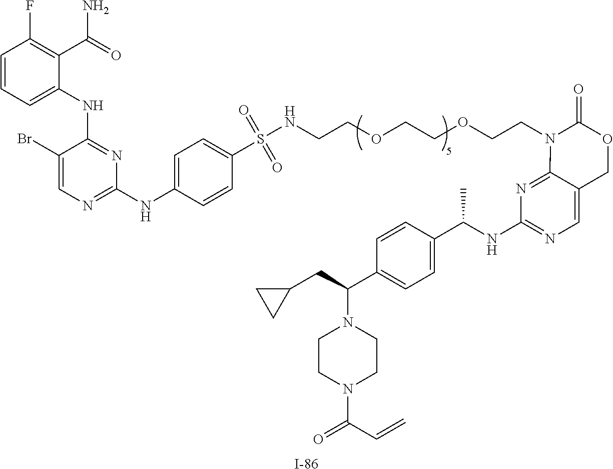

General Information: Compound 5 is described in CN113845528. Compound 7 is described in WO2018/111707.

Step 1. To a solution of 2-[2-[2-[2-[2-[2-(2-hydroxyethoxy)ethoxy]ethoxy]ethoxy]ethoxy]ethoxy]ethyl 4-methylbenzenesulfonate (4.0 g, 8.32 mmol, 1 equiv) in DMF (40 mL) was added K₂CO₃ (2.3 g, 16.6 mmol, 2.0 equiv) and 4-nitrobenzenesulfonamide (1.7 g, 8.32 mmol, 1.0 equiv). The mixture was stirred at 70° C. for 12 h. The residue was purified by prep-HPLC (column: Phenomenex luna C18 250*50 mm*10 μm; mobile phase: [water (NH₄HCO₃)-ACN]; B %: 20ACN %-48ACN %,17 min) to give N-[2-[2-[2-[2-[2-[2-(2-hydroxyethoxy)ethoxy]ethoxy]ethoxy]ethoxy]ethoxy]ethyl]-4-nitro-benzenesulfonamide (1.5 g, 35% yield) as a yellow oil. ¹H NMR (400 MHZ, CDCl₃): δ 8.28 (d, J=8.8 Hz, 2H), 8.09-8.01 (m, 2H), 3.68-3.65 (m, 2H), 3.62-3.50 (m, 20H), 3.46-3.39 (m, 4H), 3.12 (t, J=4.8 Hz, 2H).

Step 2. To a solution of N-[2-[2-[2-[2-[2-[2-(2-hydroxyethoxy)ethoxy]ethoxy]ethoxy]ethoxy]ethoxy]ethyl]-4-nitro-benzenesulfonamide (1.5 g, 2.94 mmol, 1.0 equiv) in DCM (30 mL) was added TosCl (672 mg, 3.53 mmol, 1.2 equiv) and TEA (892 mg, 8.81 mmol, 3.0 equiv). The mixture was stirred at 20° C. for 12 h. The residue was purified by prep-HPLC (column: Waters Xbridge C18 150*50 mm*10 μm; mobile phase: [water (NH₄HCO₃)-ACN]; B %: 37%-67%, 11 min) to give 2-[2-[2-[2-[2-[2-[2-[(4-nitrophenyl)sulfonylamino]ethoxy]ethoxy]ethoxy]ethoxy]ethoxy]ethoxy]ethyl 4-methylbenzenesulfonate (950 mg, 49% yield) as a yellow oil.

Step 3. To a solution of 7-methylsulfanyl-1,4-dihydropyrimido[4,5-d][1,3]oxazin-2-one (422 mg, 2.14 mmol, 1.5 equiv) in DMF (10 mL) was added K₂CO₃ (395 mg, 2.86 mmol, 2.0 equiv) and 2-[2-[2-[2-[2-[2-[2-[(4-nitrophenyl)sulfonylamino]ethoxy]ethoxy]ethoxy]ethoxy]ethoxy]ethoxy]ethyl 4-methylbenzenesulfonate (950 mg, 1.43 mmol, 1.0 equiv). The mixture was stirred at 40° C. for 12 h. The residue was purified by prep-HPLC (column: Phenomenex luna C18 150*40 mm*15 μm; mobile phase: [water (FA)-ACN]; B %: 35%-65%, 10 min) to give N-[2-[2-[2-[2-[2-[2-[2-(7-methylsulfanyl-2-oxo-4H-pyrimido[4,5-d][1,3]oxazin-1-yl)ethoxy]ethoxy]ethoxy]ethoxy]ethoxy]ethoxy]ethyl]-4-nitro-benzenesulfonamide (360 mg, 37% yield) as a yellow gum.

Step 4. To a solution of N-[2-[2-[2-[2-[2-[2-[2-(7-methylsulfanyl-2-oxo-4H-pyrimido[4,5-d][1,3]oxazin-1-yl)ethoxy]ethoxy]ethoxy]ethoxy]ethoxy]ethoxy]ethyl]-4-nitro-benzenesulfonamide (300 mg, 435 μmol, 1.0 equiv) in MeCN(10 mL) and H₂O (3 mL) was added Oxone (802 mg, 1.30 mmol, 3.0 equiv), the mixture was stirred at 20° C. for 12 h. The mixture was extracted with EtOAc (100 mL), the combined organic was washed with brine (30 mL*3), dried over anhydrous Na₂SO₄, the mixture was filtered and the filtrate was concentrated in vacuo to give N-[2-[2-[2-[2-[2-[2-[2-(7-methylsulfonyl-2-oxo-4H-pyrimido[4,5-d][1,3]oxazin-1-yl)ethoxy]ethoxy]ethoxy]ethoxy]ethoxy]ethoxy]ethyl]-4-nitro-benzenesulfonamide (300 mg, 96% yield) as a yellow gum.

Step 5. To a solution of tert-butyl 4-[(1S)-1-[4-[(1S)-1-aminoethyl]phenyl]-2-cyclopropyl-ethyl]piperazine-1-carboxylate (155 mg, 415 μmol, 1.0 equiv), N-[2-[2-[2-[2-[2-[2-[2-(7-methylsulfonyl-2-oxo-4H-pyrimido[4,5-d][1,3]oxazin-1-yl)ethoxy]ethoxy]ethoxy]ethoxy]ethoxy]ethoxy]ethyl]-4-nitro-benzenesulfonamide (300 mg, 416 μmol, 1.0 equiv) in DMSO (3 mL) was added CsF (189 mg, 1.25 mmol, 3.0 equiv) and DIEA (80 mg, 623 μmol, 1.5 equiv). The mixture was stirred at 60° C. for 1 h and purified by prep-HPLC (column: Phenomenex luna C18 150*40 mm*15 μm; mobile phase: [water (FA)-ACN]; B %: 20%-50%, 10 min) to give tert-butyl 4-[(1S)-2-cyclopropyl-1-[4-[(1S)-1-[1-[2-[2-[2-[2-[2-[2-[2-[(4-nitrophenyl)sulfonylamino]ethoxy]ethoxy]ethoxy]ethoxy]ethoxy]ethoxy]ethyl]-2-oxo-4H-pyrimido[4,5-d][1,3]oxazin-7-yl]amino]ethyl]phenyl]ethyl]piperazine-1-carboxylate (180 mg, 43% yield) as a yellow gum.

Step 6. To a solution of tert-butyl 4-[(1S)-2-cyclopropyl-1-[4-[(1S)-1-[1-[2-[2-[2-[2-[2-[2-[2-[(4-nitrophenyl)sulfonylamino]ethoxy]ethoxy]ethoxy]ethoxy]ethoxy]ethoxy]ethyl]-2-oxo-4H-pyrimido[4,5-d][1,3]oxazin-7-yl]amino]ethyl]phenyl]ethyl]piperazine-1-carboxylate (150 mg, 147 μmol, 1.0 equiv) in THF (10 mL) was added PtO2 (50 mg, 220 μmol, 1.5 equiv). The mixture was stirred under H₂ (15 psi) at 20° C. for 1 h. The mixture was filtered and the filtrate was concentrated to give tert-butyl 4-[(1S)-1-[4-[(1S)-1-[[1-[2-[2-[2-[2-[2-[2-[2-[(4-aminophenyl)sulfonylamino]ethoxy]ethoxy]ethoxy]ethoxy]ethoxy]ethoxy]ethyl]-2-oxo-4H-pyrimido[4,5-d][1,3]oxazin-7-yl]amino]ethyl]phenyl]-2-cyclopropyl-ethyl]piperazine-1-carboxylate (130 mg, 89% yield) as a yellow oil.

Step 7. To a solution of tert-butyl 4-[(1S)-1-[4-[(1S)-1-[1-[2-[2-[2-[2-[2-[2-[2-[(4-aminophenyl)sulfonylamino]ethoxy]ethoxy]ethoxy]ethoxy]ethoxy]ethoxy]ethyl]-2-oxo-4H-pyrimido[4,5-d][1,3]oxazin-7-yl]amino]ethyl]phenyl]-2-cyclopropyl-ethyl]piperazine-1-carboxylate (130 mg, 131 μmol, 1.0 equiv) and 2-[(5-bromo-2-chloropyrimidin-4-yl)amino]-6-fluoro-benzamide (45 mg, 131 μmol, 1.0 equiv) in EtOH (6 mL) was added HCl (12 M, 0.2 μL, 0.2 equiv) and H₂O (1 mL). The mixture was stirred at 80° C. for 12 h. The residue was purified by prep-HPLC (column: Phenomenex luna C18 250*50 mm* 15 μm; mobile phase: [water (FA)-ACN]; B %: 21%-51%, 10 min) to give 2-[[5-bromo-2-[4-[2-[2-[2-[2-[2-[2-[2-[7-[[(1S)-1-[4-[(1S)-2-cyclopropyl-1-piperazin-1-yl-ethyl]phenyl]ethyl]amino]-2-oxo-4H-pyrimido[4,5-d][1,3]oxazin-1-yl]ethoxy]ethoxy]ethoxy]ethoxy]ethoxy]ethoxy]ethylsulfamoyl]anilino]pyrimidin-4-yl]amino]-6-fluoro-benzamide (25 mg, 16% yield).

Step 8. To a solution of 2-[5-bromo-2-[4-[2-[2-[2-[2-[2-[2-[2-[7-[[(1S)-1-[4-[(1S)-2-cyclopropyl-1-piperazin-1-y]ethyl]phenyl]ethyl]amino]-2-oxo-4H-pyrimido[4,5-d][1,3]oxazin-1-yl]ethoxy]ethoxy]ethoxy]ethoxy]ethoxy]ethoxy]ethylsulfamoyl]anilino]pyrimidin-4-yl]amino]-6-fluoro-benzamide (25 mg, 21 μmol, 1.0 equiv) in THF (1.5 mL) was added aq·NaHCO₃ (0.5 mL) and prop-2-enoyl prop-2-enoate (3 mg, 21 μmol, 1.0 equiv) at 0° C. The mixture was stirred at 0° C. for 0.1 h and purified by prep-HPLC (column: Phenomenex luna C18 250*50 mm*15 μm; mobile phase: [water (FA)-ACN]; B %: 16%-46%,10 min) to give 2-[[5-bromo-2-[4-[2-[2-[2-[2-[2-[2-[2-[7-[[(1S)-1-[4-[(1S)-2-cyclopropyl-1-(4-prop-2-enoylpiperazin-1-yl) ethyl]phenyl]ethyl]amino]-2-oxo-4H-pyrimido[4,5-d][1,3]oxazin-1-yl]ethoxy]ethoxy]ethoxy]ethoxy]ethoxy]ethoxy]ethylsulfamoyl]anilino]pyrimidin-4-yl]amino]-6-fluoro-benzamide (15 mg, 52% yield). ¹H NMR (400 MHZ, CD₃OD): δ 8.61-8.40 (m, 2H), 8.38-8.27 (m, 1H), 7.92 (d, J=8.8 Hz, 2H), 7.82-7.72 (m, 2H), 7.66-7.52 (m, 2H), 7.44-7.29 (m, 4H), 7.11-7.02 (m, 1H), 6.75-6.72 (m, 1H), 6.20 (d, J=16.8 Hz, 1H), 5.75 (d, J=10.4 Hz, 1H), 5.15-5.12 (m, 1H), 4.33-4.29 (m, 2H), 3.67-3.60 (m, 20H), 3.57-3.55 (m, 8H), 3.12 (t, J=5.6 Hz, 2H), 2.52-2.45 (m, 4H), 1.69-1.57 (m, 4H), 1.31-1.17 (m, 3H), 0.48-0.31 (m, 3H), 0.12--0.03 (m, 2H). LC-MS: MS (ES⁺): RT=1.861 min, m/z=1251.3 [M+H⁺]; LCMS method 25.

### Example 66—the Synthetic Route for I-87

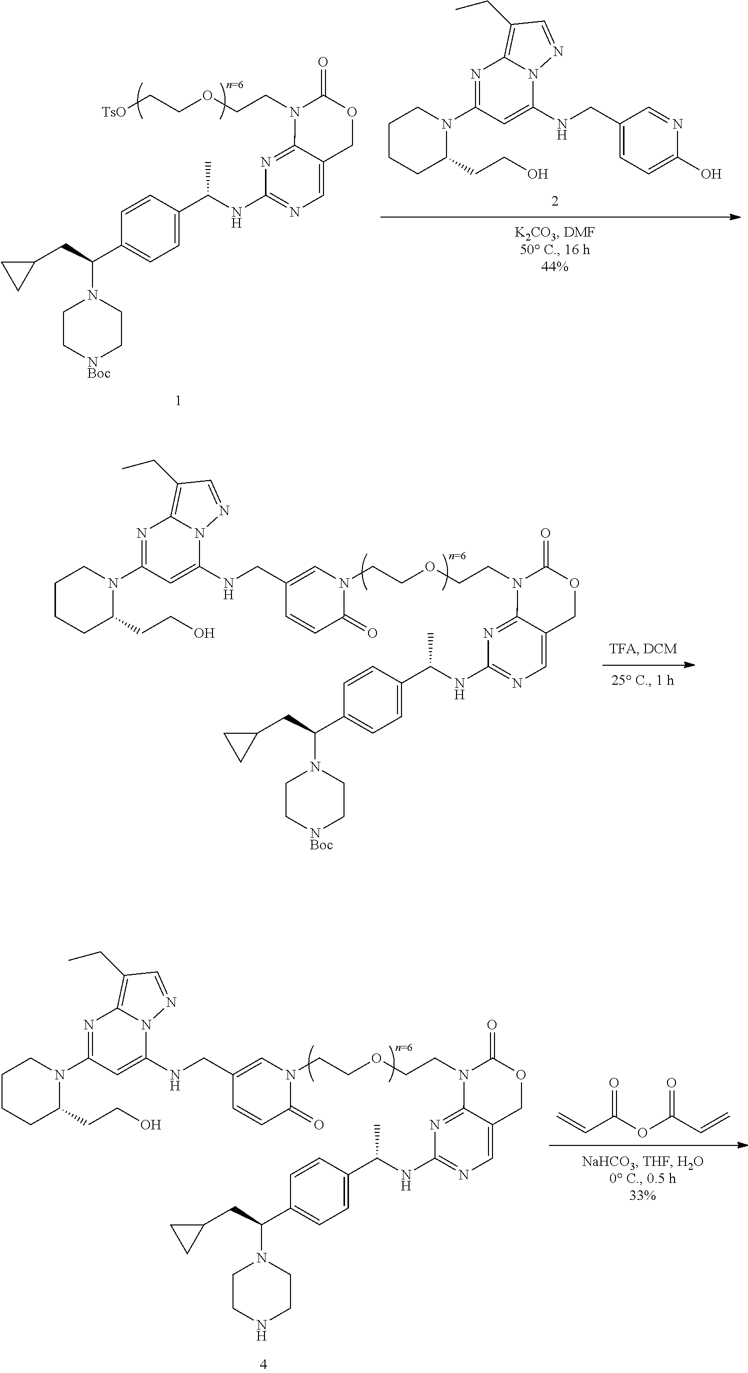

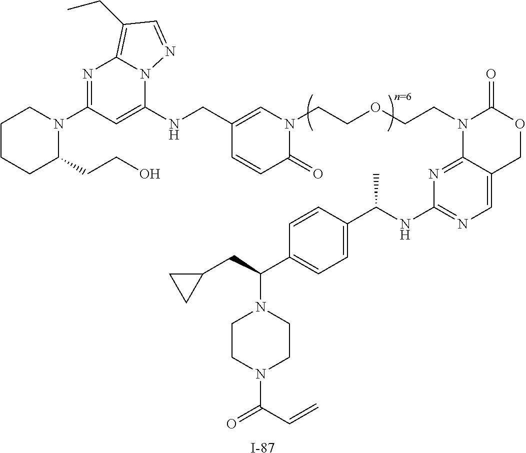

Step 1. To a solution of tert-butyl 4-[(1S)-2-cyclopropyl-1-[4-[(1S)-1-[2-oxo-1-[2-[2-[2-[2-[2-[2-[2-(p-tolylsulfonyloxy)ethoxy]ethoxy]ethoxy]ethoxy]ethoxy]ethoxy]ethyl]-4H-pyrimido[4,5-d][1,3]oxazin-7-yl]amino]ethyl]phenyl]ethyl]piperazine-1-carboxylate (165 mg, 167 μmol, 1.0 equiv) and 5-[[[3-ethyl-5-[(2S)-2-(2-hydroxyethyl)-1-piperidyl]pyrazolo[1,5-a]pyrimidin-7-yl]amino methyl]pyridin-2-ol (66 mg, 167 μmol, 1.0 equiv) in DMF (2 mL) was added K₂CO₃ (46 mg, 334 μmol, 2.0 equiv), the mixture was stirred at 50° C. for 16 h. The mixture was filtered and concentrated. The residue was purified by prep-HPLC (column: Unisil 3-100 C18 Ultra 150*50 mm*3 μm; mobile phase: [water (FA)-ACN]; B %: 15%-45%, 10 min) to give tert-butyl 4-[(1S)-2-cyclopropyl-1-[4-[(1S)-1-[1-[2-[2-[2-[2-[2-[2-[2-[5-[[[3-ethyl-5-[(2S)-2-(2-hydroxyethyl)-1-piperidyl]pyrazolo[1,5-a]pyrimidin-7-yl]amino]methyl]-2-oxo-1-pyridyl]ethoxy]ethoxy]ethoxy]ethoxy]ethoxy]ethoxy]ethyl]-2-oxo-4H-pyrimido[4,5-d][1,3]oxazin-7-yl]amino]ethyl]phenyl]ethyl]piperazine-1-carboxylate (90 mg, 44% yield).

Step 2. To a solution of tert-butyl 4-[(1S)-2-cyclopropyl-1-[4-[(1S)-1-[1-[2-[2-[2-[2-[2-[2-[2-[5-[[3-ethyl-5-[(2S)-2-(2-hydroxyethyl)-1-piperidyl]pyrazolo[1,5-a]pyrimidin-7-yl]amino]methyl]-2-oxo-1-pyridyl]ethoxy]ethoxy]ethoxy]ethoxy]ethoxy]ethoxy]ethyl]-2-oxo-4H-pyrimido[4,5-d][1,3]oxazin-7-yl]amino]ethyl]phenyl]ethyl]piperazine-1-carboxylate (90 mg, 74 μmol, 1.0 equiv) in DCM (1.5 mL) was added TFA (500 μL), the mixture was stirred at 25° C. for 1 h. The mixture was concentrated to give crude 7-[[(1S)-1-[4-[(1S)-2-cyclopropyl-1-piperazin-1-yl-ethyl]phenyl]ethyl]amino]-1-[2-[2-[2-[2-[2-[2-[2-[5-[3-ethyl-5-[(2S)-2-(2-hydroxyethyl)-1-piperidyl]pyrazolo[1,5-a]pyrimidin-7-yl]amino]methyl]-2-oxo-1-pyridyl]ethoxy]ethoxy]ethoxy]ethoxy]ethoxy]ethoxy]ethyl]-4H-pyrimido[4,5-d][1,3]oxazin-2-one (91 mg, crude, TFA salt) which was used into the next step without further purification.

Step 3. To a solution of 7-[(1S)-1-[4-[(1S)-2-cyclopropyl-1-piperazin-1-yl-ethyl]phenyl]ethyl]amino]-1-[2-[2-[2-[2-[2-[2-[2-[5-[3-ethyl-5-[(2S)-2-(2-hydroxyethyl)-1-piperidyl]pyrazolo[1,5-a]pyrimidin-7-yl]amino]methyl]-2-oxo-1-pyridyl]ethoxy]ethoxy]ethoxy]ethoxy]ethoxy]ethoxy]ethyl]-4H-pyrimido[4,5-d][1,3]oxazin-2-one (91 mg, 74 μmol, 1.0 equiv, TFA salt) and NaHCO₃ (1 M, 2 mL, 26.8 equiv) in THF (6 mL) was added prop-2-enoyl prop-2-enoate (9 mg, 74 μmol, 1.0 equiv) at 0° C., the mixture was stirred at 0° C. for 0.5 h. The mixture was diluted with water (10 mL) and extracted with EtOAc (20 mL*3). The combined organic layers were washed with brine (30 mL), dried over Na₂SO₄, filtered and concentrated. The residue was purified by prep-HPLC (column: Unisil 3-100 C18 Ultra 150*50 mm*3 μm; mobile phase: [water (FA)-ACN]; B %: 10%-40%, 10 min) to give 7-[[(1S)-1-[4-[(1S)-2-cyclopropyl-1-(4-prop-2-enoylpiperazin-1-yl) ethyl]phenyl]ethyl]amino]-1-[2-[2-[2-[2-[2-[2-[2-[5-[[3-ethyl-5-[(2S)-2-(2-hydroxyethyl)-1-piperidyl]pyrazolo[1,5-a]pyrimidin-7-yl]amino]methyl]-2-oxo-1-pyridyl]ethoxy]ethoxy]ethoxy]ethoxy]ethoxy]ethoxy]ethyl]-4H-pyrimido[4,5-d][1,3]oxazin-2-one (30 mg, 33% yield). ¹H NMR (400 MHZ, DMSO-d₆): δ 7.99 (s, 1H), 7.91-7.76 (m, 1H), 7.75-7.60 (m, 3H), 7.56-7.48 (m, 1H), 7.34-7.25 (m, 2H), 7.20-7.12 (m, 2H), 6.75-6.62 (m, 1H), 6.39-6.32 (m, 1H), 6.07-5.98 (m, 1H), 5.66-5.52 (m, 2H), 5.09 (s, 2H), 4.66-4.51 (m, 1H), 4.35-4.17 (m, 3H), 4.11-3.81 (m, 4H), 3.64-3.60 (m, 2H), 3.50-3.39 (m, 30H), 2.88-2.76 (m, 1H), 2.47-2.43 (m, 2H), 2.29-2.23 (m, 4H), 1.89-1.77 (m, 1H), 1.76-1.50 (m, 9H), 1.45-1.40 (m, 3H), 1.38-1.30 (m, 1H), 1.19-1.13 (m, 3H), 0.43-0.34 (m, 1H), 0.32-0.19 (m, 2H), 0.03-0.11 (m, 2H). LC-MS: MS (ES⁺): RT =2.157 min, m/z=1163.6 [M+H⁺]; LCMS method 10.

### Example 67—the Synthetic Route for I-88

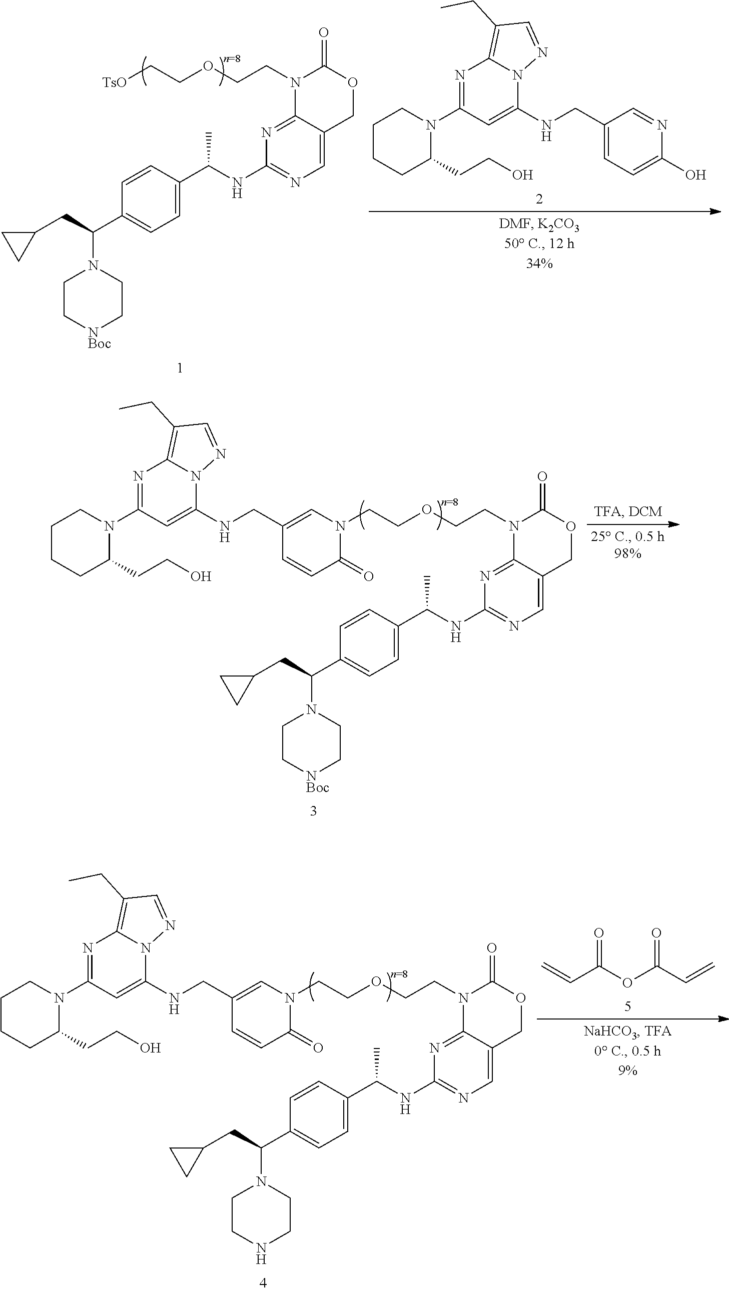

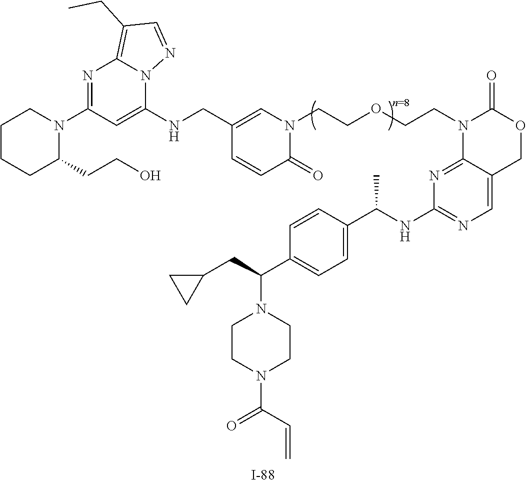

General Information: Compound 5 is described in U.S. Pat. No. 9,266,811.

Step 1. To a solution of tert-butyl 4-[(1S)-2-cyclopropyl-1-[4-[(1S)-1-[[2-oxo-1-[2-[2-[2-[2-[2-[2-[2-[2-[2-(p-tolylsulfonyloxy)ethoxy]ethoxy]ethoxy]ethoxy]ethoxy]ethoxy]ethoxy]ethoxy]ethyl]-4H-pyrimido[4,5-d]1,3]oxazin-7-yl]amino]ethyl]phenyl]ethyl]piperazine-1-carboxylate (190 mg, 177 μmol, 1 equiv) and 5-[[[3-ethyl-5-[(2S)-2-(2-hydroxyethyl)-1-piperidyl]pyrazolo[1,5-a]pyrimidin-7-yl]amino]methyl]pyridin-2-ol (70 mg, 177 μmol, 1 equiv) in DMF (2 mL) was added K₂CO₃ (48 mg, 354 μmol, 2 equiv). The mixture was stirred at 50° C. for 12 h. The mixture filtered and concentrated. The residue was purified by prep-HPLC (column: Unisil 3-100 C18 Ultra 150*50 mm*3 μm; mobile phase: [water (FA)-ACN]; B %: 15%-45%, 10 min) to give compound tert-butyl 4-[(1S)-2-cyclopropyl-1-[4-[(1S)-1-[[1-[2-[2-[2-[2-[2-[2-[2-[2-[2-[[5-[[[3-ethyl-5-[(2S)-2-(2-hydroxyethyl)-1-piperidyl]pyrazolo]1,5-a]pyrimidin-7-yl]amino]methyl]-2-pyridyl]oxy]ethoxy]ethoxy]ethoxy]ethoxy]ethoxy]ethoxy]ethoxy]ethoxy]ethyl]-2-oxo-4H-pyrimido[4,5-d][1,3]oxazin-7-yl]amino]ethyl]phenyl]ethyl]piperazine-1-carboxylate (70 mg, 34% yield).

Step 2. To a solution of tert-butyl 4-[(1S)-2-cyclopropyl-1-[4-[(1S)-1-[[1-[2-[2-[2-[2-[2-[12-[2-[2-[2-[5-[[[3-ethyl-5-[(2S)-2-(2-hydroxyethyl)-1-piperidyl]pyrazolo[1,5-a]pyrimidin-7-yl]amino]methyl]-2-oxo-1-pyridyl]ethoxy]ethoxy]ethoxy]ethoxy]ethoxy]ethoxy]ethoxy]ethoxy]ethyl]-2-oxo-4H-pyrimido[4,5-d][1,3]oxazin-7-yl]amino]ethyl]phenyl]ethyl]piperazine-1-carboxylate (80 mg, 61 μmol, 1 equiv) in DCM (3 mL) was added TFA (1 mL). The mixture was stirred at 25° C. for 0.5 h. The mixture filtered and concentrated. The residue was used for next step directly to give 7-[[(1S)-1-[4-[(1S)-2-cyclopropyl-1-piperazin-1-yl-ethyl]phenyl]ethyl]amino]-1-[2-[2-[2-[2-[2-[2-[2-[2-[2-[5-[[[3-ethyl-5-[(2S)-2-(2-hydroxyethyl)-1-piperidyl]pyrazolo[1,5-a]pyrimidin-7-yl]amino]methyl]-2-oxo-1-pyridyl]ethoxy]ethoxy]ethoxy]ethoxy]ethoxy]ethoxy]ethoxy]ethoxy]ethyl]-4H-pyrimido[4,5-d][1,3]oxazin-2-one (80 mg, 98% yield).

Step 3. To a solution of prop-2-enoyl prop-2-enoate (7 mg, 61 μmol, 1 equiv) in THF (2 mL) was added NaHCO₃ (1 M, 1.59 mL, 26 equiv) and 7-[[(1S)-1-[4-[(1S)-2-cyclopropyl-1-piperazin-1-yl-ethyl]phenyl]ethyl]amino]-1-[2-[2-[2-[2-[2-[2-[2-[2-[2-[5-[[[3-ethyl-5-[(2S)-2-(2-hydroxyethyl)-1-piperidyl]pyrazolo]1,5-a]pyrimidin-7-yl]amino]methyl]-2-oxo-1-pyridyl]ethoxy]ethoxy]ethoxy]ethoxy]ethoxy]ethoxy]ethoxy]ethoxy]ethyl]-4H-pyrimido]4,5-d][1,3]oxazin-2-one (80 mg, 61 μmol, 1 equiv, TFA salt). The mixture was stirred at 0° C. for 0.5 h. The reaction mixture was diluted with water (5 mL) and extracted with ethyl acetate (5 mL*3). The combined organic layers were washed with brine (10 mL), dried over sodium sulfate, filtered and concentrated to give 7-[[(1S)-1-[4-[(1S)-2-cyclopropyl-1-(4-prop-2-enoylpiperazin-1-yl) ethyl]phenyl]ethyl]amino]-1-[2-[2-[2-[2-[2-[2-[2-[2-[2-[5-[[[3-ethyl-5-[(2S)-2-(2-hydroxyethyl)-1-piperidyl]pyrazolo[1,5-a]pyrimidin-7-yl]amino]methyl]-2-oxo-1-pyridyl]ethoxy]ethoxy]ethoxy]ethoxy]ethoxy]ethoxy]ethoxy]ethoxy]ethyl]-4H-pyrimido[4,5-d][1,3]oxazin-2-one (6 mg, 9% yield). ¹H NMR (400 MHZ, DMSO): δ 8.36 (s, 1H), 8.01 (s, 1H), 7.94-7.80 (m, 1H), 7.76-7.69 (m, 2H), 7.66 (s, 1H), 7.55-7.52 (m, 1H), 7.33 (d, J=8.1 Hz, 2H), 7.19 (d, J=8.1 Hz, 2H), 6.74-6.67 (m, 1H), 6.39 (d, J=9.3 Hz, 1H), 6.06-6.02 (m, 1H), 5.66-5.56 (m, 2H), 5.16-4.93 (m, 3H), 4.79 (s, 1H), 4.61 (s, 1H), 4.48-4.19 (m, 4H), 4.17-3.88 (m, 5H), 3.63 (t, J=5.6 Hz, 5H), 3.49 (d, J=4.4 Hz, 35H), 2.94-2.76 (m, 1H), 1.90-1.78 (m, 1H), 1.75-1.51 (m, 9H), 1.47-1.37 (m, 4H), 1.18 (t, J=7.5 Hz, 3H), 0.43-0.23 (m, 3H), 0.05--0.08 (m, 2H). LC-MS: MS (ES⁺): RT=2.181 min, m/z=626.4 [M+H+]; LCMS method 25.

### Example 68—the Synthetic Route for I-89

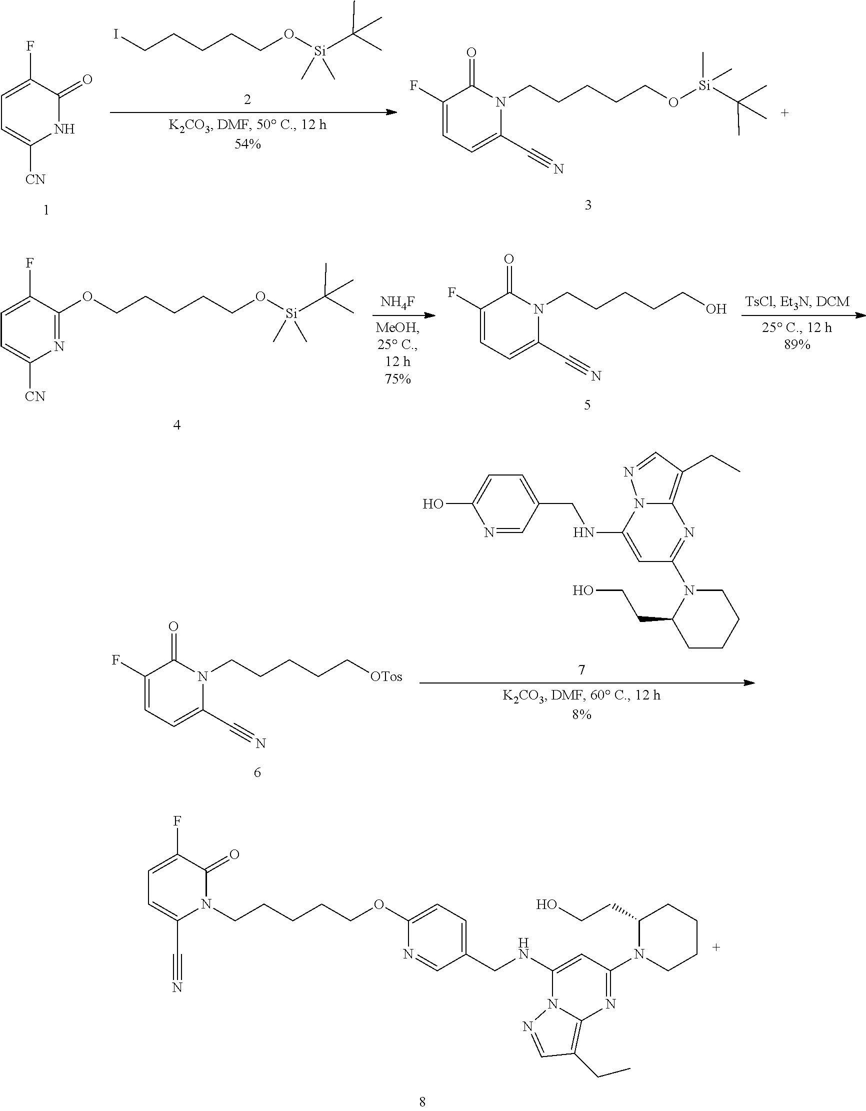

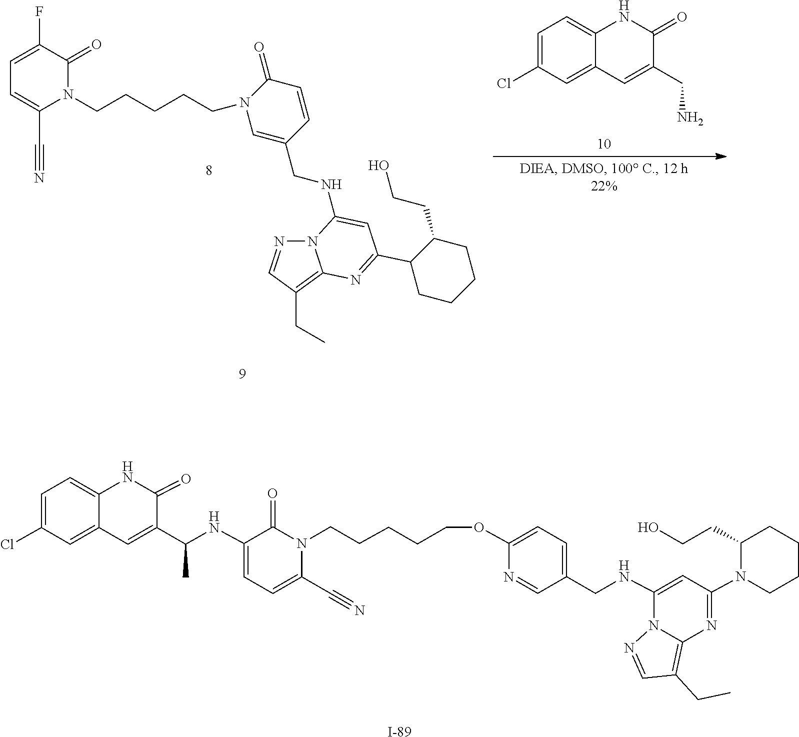

General Information: Synthetic route for compound 1 is provided in Journal of Medicinal Chemistry, 2020, vol. 63, #4, p. 1612-1623. Synthetic route for compound 2 is provided in Journal of Organic Chemistry, 2009, vol. 74, #20, p. 7982-7985. Synthetic route for compound 10 is provided in Journal of Medicinal Chemistry, 2019, vol. 62, #14, p. 6575-6596.

Step 1. A mixture of tert-butyl-(5-iodopentoxy)-dimethyl-silane (7.13 g, 21.7 mmol, 2.0 equiv), 5-fluoro-6-oxo-1H-pyridine-2-carbonitrile (1.50 g, 10.9 mmol, 1.0 equiv) and K₂CO₃ (4.50 g, 32.6 mmol, 3.0 equiv) in DMF (30 mL) was stirred at 50° C. for 12 h under N₂ atmosphere. The reaction mixture was quenched by addition water 150 mL at 25° C., and then diluted with Ethyl acetate 50 mL and extracted with Ethyl acetate (150 mL*3). The combined organic layers were washed with brine 150 mL, dried over Na₂SO₄, filtered and concentrated under reduced pressure to give a residue. The residue was purified by column chromatography (SiO2, Petroleum ether/Ethyl acetate=10/1 to 1/1). Compound 1-[5-[tert-butyl(dimethyl)silyl]oxypentyl]-5-fluoro-6-oxo-pyridine-2-carbonitrile (800 mg, 2.36 mmol, 22% yield) was obtained as a white solid. ¹H NMR: (400 MHZ, CHLOROFORM-d) δ=7.08 (t, J=8.0 Hz, 1H), 6.81-6.69 (m, 1H), 4.31-4.17 (m, 2H), 3.73-3.56 (m, 4H), 1.88-1.72 (m, 2H), 1.62-1.54 (m, 4H), 1.51-1.39 (m, 3H), 0.96-0.84 (m, 11H), 0.10-0.01 (m, 8H). 6-[5-[tert-butyl(dimethyl)silyl]oxypentoxy]-5-fluoro-pyridine-2-carbonitrile (2.00 g, 5.91 mmol, 54% yield) was obtained as a white solid. ¹H NMR: (400 MHZ, CHLOROFORM-d) δ=7.43-7.35 (m, 1H), 7.34-7.28 (m, 1H), 4.42 (t, J=6.8 Hz, 2H), 3.64 (t, J=6.0 Hz, 2H), 1.89-1.80 (m, 2H), 1.65-1.46 (m, 5H), 0.89 (s, 9H), 0.05 (s, 6H).

Step 2. A mixture of 1-[5-[tert-butyl(dimethyl)silyl]oxypentyl]-5-fluoro-6-oxo-pyridine-2-carbonitrile (800 mg, 2.36 mmol, 1.0 equiv) and NH₄F (400 mg, 10.8 mmol, 4.6 equiv) in MeOH (10 mL) was stirred at 25° C. for 12 hr under N₂ atmosphere. The reaction mixture was filtered and concentrated under reduced pressure to give a residue. The residue was purified by column chromatography (SiO₂, Petroleum ether/Ethyl acetate=10/1 to 2/1). Compound 5-fluoro-1-(5-hydroxypentyl)-6-oxo-pyridine-2-carbonitrile (400 mg, 1.78 mmol, 75% yield) was obtained as a white solid. ¹H NMR: (400 MHZ, CHLOROFORM-d) δ=7.09 (t, J=8.0 Hz, 1H), 6.81-6.71 (m, 1H), 4.29-4.20 (m, 2H), 3.68 (t, J=6.4 Hz, 2H), 1.91-1.80 (m, 2H), 1.73-1.63 (m, 2H), 1.56-1.44 (m, 2H).

Step 3. A mixture of 5-(6-cyano-3-fluoro-2-oxo-1-pyridyl)pentyl 4-methyl benzenesulfonate (300 mg, 793 μmol, 1.0 equiv), 5-[[[3-ethyl-5-[(2S)-2-(2-hydroxyethyl)-1-piperidyl]pyrazolo[1,5-a]pyrimidin-7-yl]amino]methyl]pyridin-2-ol (449 mg, 1.13 mmol, 1.4 equiv) in NMP (10 mL) was added K₂CO₃ (330 mg, 2.39 mmol, 3.0 equiv) and the mixture was stirred at 60° C. for 12 h under N₂ atmosphere. The reaction mixture was poured into water 50 mL at 0° C., and then diluted with Ethyl acetate 50 mL and extracted with ethyl acetate (50 mL×3). The combined organic layers were washed with brine 10 mL, dried over Na₂SO₄, filtered and concentrated under reduced pressure to give a residue. The residue was purified by prep-TLC (SiO2, DCM: MeOH=15:1). Compound 1-[5-[[5-[[[3-ethyl-5-[(2S)-2-(2-hydroxyethyl)-1-piperidyl]pyrazolo[1,5-a]pyrimidin-7-yl]amino]methyl]-2-pyridyl Joxy]pentyl]-5-fluoro-6-oxo-pyridine-2-carbonitrile (45.0 mg, 54.5 μmol, 7% yield, 73% purity) was obtained as a white gum. ¹H NMR: (400 MHz, DMSO-d₆) δ=8.22 (d, J=1.8 Hz, 1H), 7.84 (s, 1H), 7.78-7.73 (m, 1H), 7.65 (s, 1H), 7.55-7.48 (m, 1H), 7.23-7.15 (m, 1H), 6.75 (d, J=8.7 Hz, 1H), 4.72 (s, 1H), 4.62-4.53 (m, 1H), 4.45 (br d, J=6.0 Hz, 2H), 4.32-4.25 (m, 1H), 4.21 (br t, J=6.5 Hz, 2H), 4.08 (br t, J=7.5 Hz, 2H), 4.05-3.95 (m, 1H), 3.60 (s, 1H), 2.80 (s, 1H), 1.81-1.51 (m, 17H), 1.16 (t, J=7.5 Hz, 4H). Compound 1-[5-[5-[[3-ethyl-5-[(2S)-2-(2-hydroxyethyl)-1-piperidyl]pyrazolo[1,5-a]pyrimidin-7-yl]amino]methyl]-2-oxo-1-pyridyl]pentyl]-5-fluoro-6-oxo-pyridine-2-carbonitrile (65.0 mg, 63.6 μmol, 8% yield, 59% purity) was obtained as a white gum. ¹H NMR: (400 MHZ, DMSO-d₆) δ=7.83-7.73 (m, 2H), 7.64 (s, 1H), 7.57-7.47 (m, 2H), 7.23-7.18 (m, 1H), 6.35 (d, J=9.2 Hz, 1H), 5.56 (s, 1H), 4.78 (s, 1H), 4.62-4.53 (m, 1H), 4.40-4.30 (m, 1H), 4.26-4.19 (m, 2H), 4.10-4.04 (m, 2H), 4.03-3.95 (m, 1H), 3.91-3.83 (m, 1H), 3.82-3.72 (m, 1H), 3.60 (br t, J=6.4 Hz, 3H), 2.87-2.75 (m, 1H), 1.81-1.53 (m, 17H), 1.20-1.12 (m, 4H).

Step 4. A mixture of 1-[5-[[5-[[3-ethyl-5-[(2S)-2-(2-hydroxyethyl)-1-piperidyl]pyrazolo[1,5-a]pyrimidin-7-yl]amino]methyl]-2-pyridyl]oxy]pentyl]-5-fluoro-6-oxo-pyridine-2-carbonitrile (40.0 mg, 66.4 μmol, 1.0 equiv), 3-[(1S)-1-aminoethyl]-6-chloro-1H-quinolin-2-one (80.0 mg, 309 μmol, 4.7 equiv, HCl), DIPEA (89.0 mg, 689 μmol, 120 μL, 10.4 equiv) in DMSO (0.5 mL) was degassed and purged with N₂ for 3 times, and then the mixture was stirred at 100° C. for 12 h under N₂ atmosphere. The reaction mixture was concentrated under reduced pressure to give a residue. The residue was purified by prep-HPLC (FA condition; column: Unisil 3-100 C18 Ultra 150*50 mm*3 μm; mobile phase: [water (FA)-ACN]; B %: 35%-65%,7 min). Compound 5-[(1S)-1-(6-chloro-2-oxo-1H-quinolin-3-yl) ethyl]amino]-1-[5-[[5-[[3-ethyl-5-[(2S)-2-(2-hydroxyethyl)-1-piperidyl]pyrazolo[1,5-a]pyrimidin-7-yl]amino methyl]-2-pyridyl]oxy]pentyl]-6-oxo-pyridine-2-carbonitrile (11.7 mg, 14.4 μmol, 22% yield, 99% purity) was obtained as a yellow solid. ¹H NMR: (400 MHZ, METHANOL-d4) δ=8.18 (d, J=2.0 Hz, 1H), 7.77 (s, 1H), 7.76-7.71 (m, 1H), 7.63 (s, 1H), 7.62 (d, J=2.0 Hz, 1H), 7.48-7.42 (m, 1H), 7.31 (d, J=8.8 Hz, 1H), 6.83 (d, J=8.0 Hz, 1H), 6.77 (d, J=8.4 Hz, 1H), 6.02 (d, J=7.6 Hz, 1H), 5.51 (s, 1H), 4.81-4.75 (m, 2H), 4.62 (s, 4H)4.53 (s, 2H), 4.28 (t, J=6.0 Hz, 2H), 4.20 (br t, J=7.2 Hz, 2H), 4.05-3.96 (m, 1H), 3.58-3.51 (m, 1H), 2.99 (br t, J=13.6 Hz, 1H), 2.59-2.51 (m, 2H), 2.13-2.02 (m, 1H), 1.90-1.80 (m, 4H), 1.76-1.62 (m, 6H), 1.58 (d, J=6.8 Hz, 3H), 1.57-1.52 (m, 2H), 1.22 (t, J=7.6 Hz, 3H). LC-MS: MS (ES⁺): RT=2.354 min, m/z=805.3 [M+H⁺], LCMS method 25.

### Example 69—the Synthetic Route for I-90

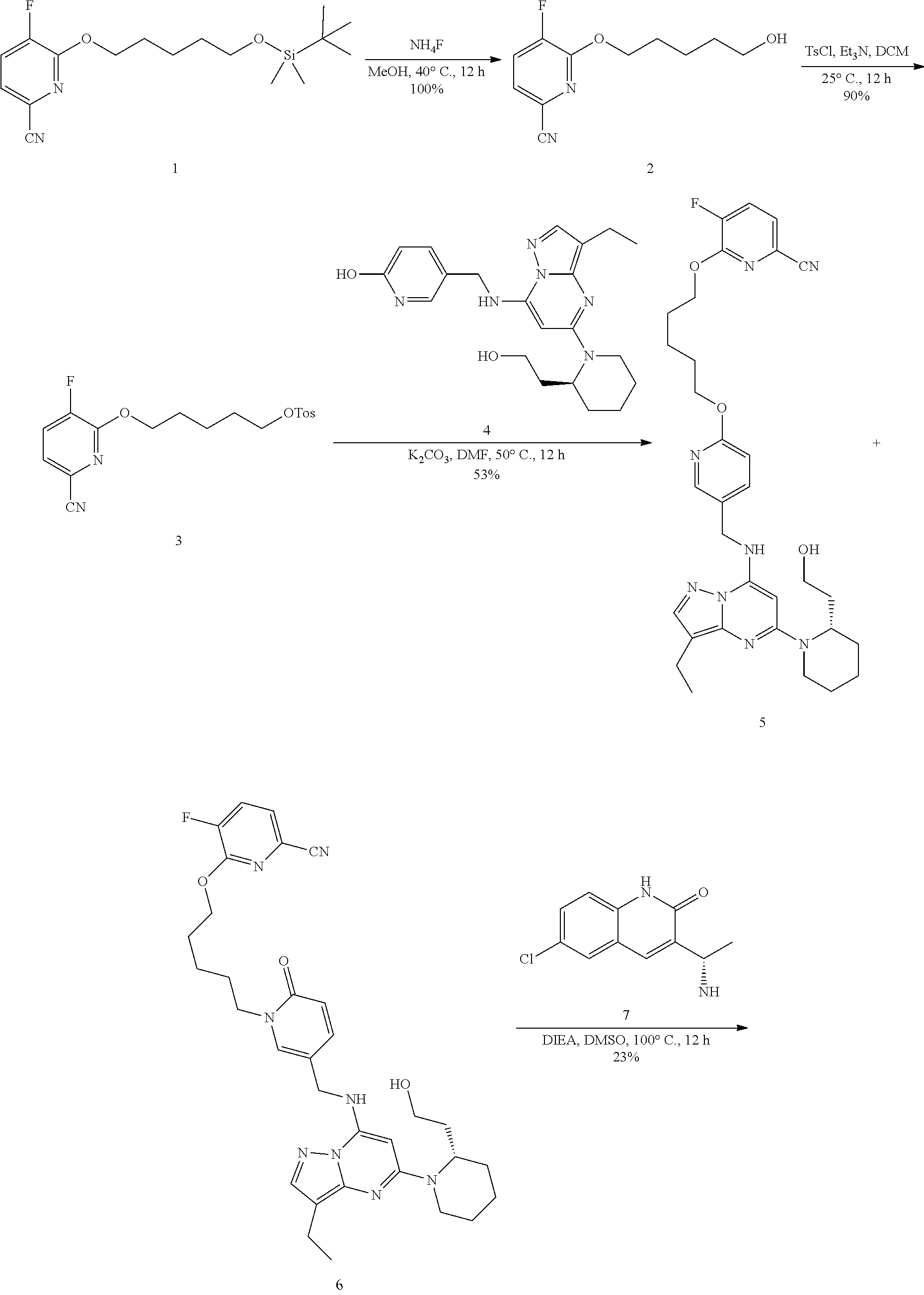

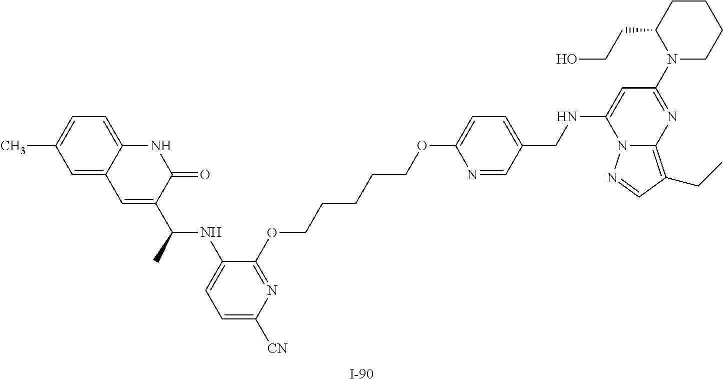

General Information: Synthetic route for compound 7 is provided in Journal of Medicinal Chemistry, 2019, vol. 62, #14, p. 6575-6596.

Step 1. A mixture of 6-[5-[tert-butyl(dimethyl)silyl]oxypentoxy]-5-fluoro-pyridine-2-carbonitrile (1.00 g, 2.95 mmol, 1.0 equiv) and NH₄F (500 mg, 13.5 mmol, 4.6 equiv) in MeOH (20 mL) was stirred at 40° C. for 12 h under N₂ atmosphere. The reaction mixture was filtered and concentrated under reduced pressure to give a residue. The residue was purified by column chromatography (SiO₂, Petroleum ether/Ethyl acetate=10/1 to 1/1). Compound 5-fluoro-6-(5-hydroxypentoxy) pyridine-2-carbonitrile (660 mg, 2.94 mmol, 100% yield) was obtained as a Colorless gum.

Step 2. A mixture of 5-fluoro-6-(5-hydroxypentoxy) pyridine-2-carbonitrile (660 mg, 2.94 mmol, 1.0 equiv), TosCl (2.76 g, 14.5 mmol, 4.9 equiv) and Et₃N(1.82 g, 18.0 mmol, 2.50 mL, 6.1 equiv) in DCM (20 mL) was stirred at 25° C. for 12 h under N₂ atmosphere. The reaction mixture was filtered and concentrated under reduced pressure to give a residue. The residue was purified by column chromatography (SiO₂, Petroleum ether/Ethyl acetate=10/1 to 1/1). Compound 5-[(6-cyano-3-fluoro-2-pyridyl) oxy]pentyl 4-methylbenzenesulfonate (1.00 g, 2.64 mmol, 90% yield) was obtained as a white solid.

Step 3. 5-[(6-cyano-3-fluoro-2-pyridyl) oxy]pentyl 4-methylbenzenesulfonate (400 mg, 1.06 mmol, 1.0 equiv), 5-[[[3-ethyl-5-[(2S)-2-(2-hydroxyethyl)-1-piperidyl]pyrazolo[1,5-a]pyrimidin-7-yl]amino methyl]pyridin-2-ol (629 mg, 1.59 mmol, 1.5 equiv), K₂CO₃ (438 mg, 3.17 mmol, 3.0 equiv) in NMP (2 mL) was stirred at 50° C. for 12 h under N₂. The reaction mixture was poured into water 50 mL at 0° C., and then diluted with Ethyl acetate 50 mL and extracted with Ethyl acetate (50 mL×3). The combined organic layers were washed with brine 10 mL, dried over Na₂SO₄, filtered and concentrated under reduced pressure to give a residue. The residue was purified by column chromatography (SiO₂, Petroleum ether/Ethyl acetate=1/1 to 0/1). Compound 6-[5-[[5-[3-ethyl-5-[(2S)-2-(2-hydroxyethyl)-1-piperidyl]pyrazolo[1,5-a]pyrimidin-7-yl]amino methyl]-2-pyridyl]oxy]pentoxy]-5-fluoro-pyridine-2-carbonitrile (230 mg, 382 μmol, 36% yield) was obtained as a yellow solid. ¹H NMR: (400 MHZ, DMSO-d₆)8 =8.22 (d, J=2.0 Hz, 1H), 7.93-7.87 (m, 1H), 7.84 (br t, J=6.4 Hz, 1H), 7.77-7.71 (m, 2H), 7.65 (s, 1H), 6.75 (d, J=8.4 Hz, 1H), 5.58 (s, 1H), 4.72 (t, J=5.6 Hz, 1H), 4.56 (br s, 1H), 4.45 (br d, J=6.4 Hz, 2H), 4.37 (t, J=6.4 Hz, 2H), 4.31-4.19 (m, 3H), 3.43-3.37 (m, 2H), 2.87-2.75 (m, 1H), 2.47-2.43 (m, 2H), 1.85-1.71 (m, 5H), 1.69-1.42 (m, 9H), 1.18-1.14 (m, 3H). Compound 6-[5-[5-[[[3-ethyl-5-[(2S)-2-(2-hydroxyethyl)-1-piperidyl]pyrazolo[1,5-a]pyrimidin-7-yl]amino methyl]-2-oxo-1-pyridyl]pentoxy]-5-fluoro-pyridine-2-carbonitrile (340 mg, 564 μmol, 53% yield) was obtained as a white solid. ¹H NMR: (400 MHZ, DMSO-d6) δ=7.94-7.88 (m, 1H), 7.81 (d, J=2.0 Hz, 1H), 7.78-7.72 (m, 2H), 7.64 (s, 1H), 7.53-7.47 (m, 1H), 6.35 (d, J=9.2 Hz, 1H), 5.56 (s, 1H), 4.78 (s, 1H), 4.62-4.51 (m, 1H), 4.35 (br t, J=6.8 Hz, 3H), 4.23 (br t, J=5.2 Hz, 2H), 3.88 (br s, 2H), 3.81 (br s, 2H), 2.17 (t, J=8.0 Hz, 2H), 1.94-1.86 (m, 3H), 1.83-1.74 (m, 4H), 1.71-1.52 (m, 9H), 1.45-1.33 (m, 4H), 1.18-1.14 (m, 3H).

Step 4. 3—[(1S)-1-aminoethyl]-6-chloro-1H-quinolin-2-one (200 mg, 772 μmol, 4.7 equiv, HCl), 6-[5-[5-[[3-ethyl-5-[(2S)-2-(2-hydroxyethyl)-1-piperidyl]pyrazolo[1,5-a]pyrimidin-7-yl]amino methyl]-2-pyridyl]oxy]pentoxy]-5-fluoro-pyridine-2-carbonitrile (100 mg, 166 μmol, 1.0 equiv), DIEA (223 mg, 1.72 mmol, 300 μL, 10.4 equiv) in DMSO (3 mL) was heated to 100° C. for 12 h under N₂. The reaction mixture was concentrated under reduced pressure to give a residue. The residue was purified by prep-HPLC (FA condition; column: Unisil 3-100 C18 Ultra 150*50 mm*3 μm; mobile phase: [water (FA)-ACN]; B %: 40%-70%,7 min). Compound 5-[[(1S)-1-(6-chloro-2-oxo-1H-quinolin-3-yl) ethyl]amino]-6-[5-[5-[[[3-ethyl-5-[(2S)-2-(2-hydroxyethyl)-1-piperidyl]pyrazolo[1,5-a]pyrimidin-7-yl]amino methyl]-2-pyridyl]oxy]pentoxy]pyridine-2-carbonitrile (31.9 mg, 38.0 μmol, 23% yield, 96% purity) was obtained. ¹H NMR: (400 MHZ, METHANOL-d₄) δ=8.15 (d, J=2.0 Hz, 1H), 7.78 (s, 1H), 7.72-7.68 (m, 1H), 7.63 (s, 1H), 7.57 (d, J=2.4 Hz, 1H), 7.44-7.39 (m, 1H), 7.27 (d, J=8.8 Hz, 1H), 7.15 (d, J=7.6 Hz, 1H), 6.73 (d, J=8.4 Hz, 1H), 6.53 (d, J=8.0 Hz, 1H), 5.50 (s, 1H), 4.82-4.76 (m, 2H), 4.52 (s, 2H), 4.44 (t, J=6.4 Hz, 2H), 4.29 (t, J=6.4 Hz, 2H), 4.03-3.95 (m, 1H), 3.57-3.50 (m, 1H), 3.42-3.34 (m, 1H), 3.02-2.94 (m, 1H), 2.59-2.51 (m, 2H), 2.11-2.02 (m, 1H), 1.97-1.83 (m, 4H), 1.75-1.60 (m, 8H), 1.57 (d, J=6.8 Hz, 3H), 1.54-1.44 (m, 1H), 1.22 (t, J=7.6 Hz, 3H). LC-MS: MS (ES⁺): RT=2.508 min, m/z=805.3 [M+H⁺], LCMS method 25.

### Example 70—the Synthetic Route for I-91

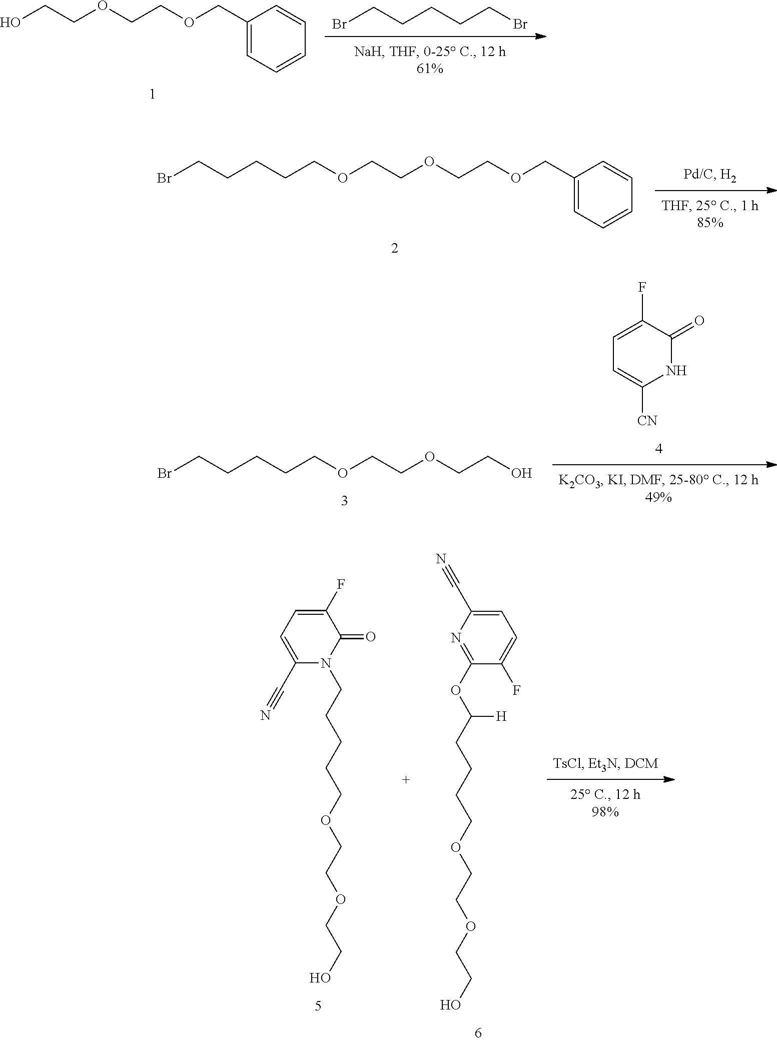

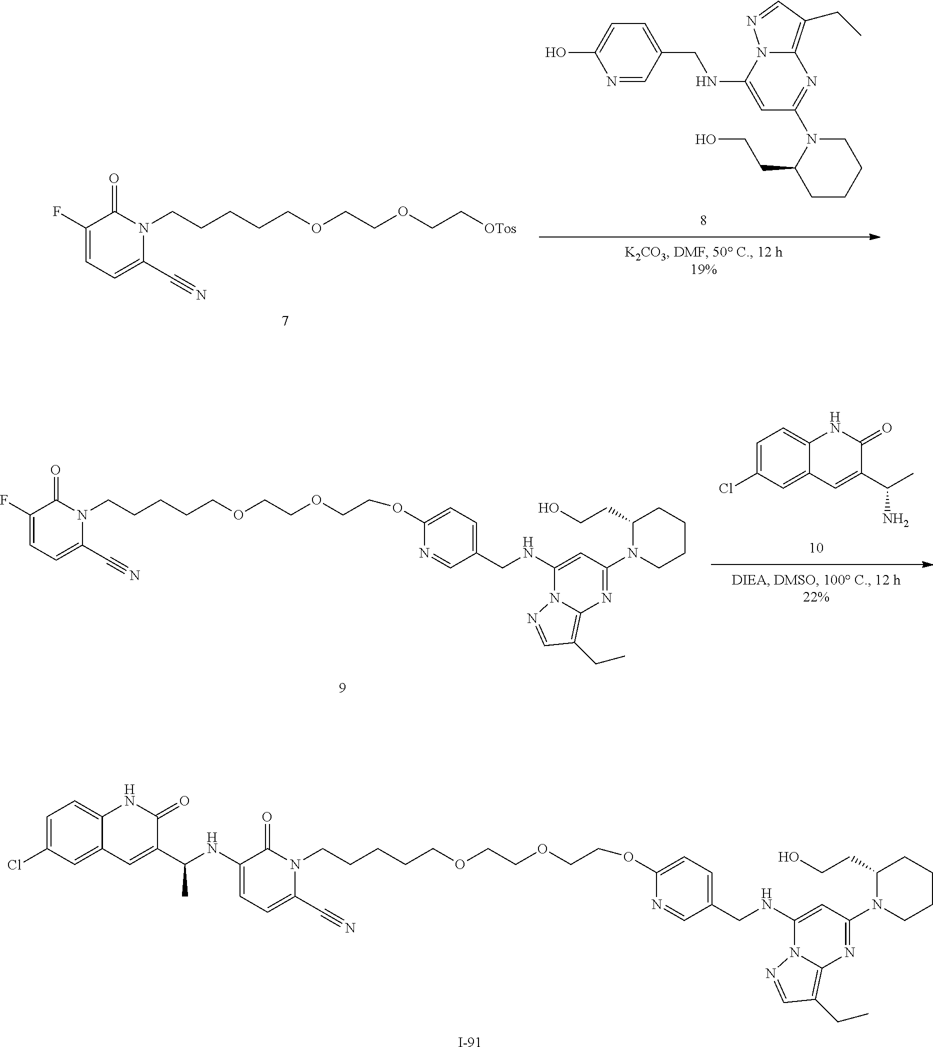

General Information: Synthetic route for compound 1 is described in Synthetic Communications, 1986, vol. 16, #1, p. 19-26. Synthetic route for compound 4 is described in Journal of Medicinal Chemistry, 2020, vol. 63, #4, p. 1612-1623. Synthetic route for compound 10 is described in Journal of Medicinal Chemistry, 2019, vol. 62, #14, p. 6575-6596.

Step 1. To a solution of 2-(2-benzyloxyethoxy) ethanol (5.00 g, 25.5 mmol, 1.0 equiv) in THF (60 mL) was added NaH (2.04 g, 51.0 mmol, 60% purity, 2.0 equiv) at 0° C. The mixture was stirred at 30° C. for 0.5 h. Then 1,5-dibromopentane (11.7 g, 51.0 mmol, 6.89 mL, 2.0 equiv) was added and stirred at 30° C. for 11.5 h. The reaction mixture was partitioned between H₂O 150 mL and EA 200 mL. The organic phase was separated, dried over Na₂SO₄, filtered and concentrated under reduced pressure to give a residue. The residue was purified by column chromatography (SiO₂, Petroleum ether/Ethyl acetate=10/1 to 1/1). Compound 2-[2-(6-bromohexoxy)ethoxy]ethoxymethylbenzene (5.60 g, 15.6 mmol, 61% yield) was obtained as a yellow oil.

Step 2. To a solution of 2-[2-(6-bromohexoxy)ethoxy]ethoxymethylbenzene (5.50 g, 15.3 mmol, 1.0 equiv) in THF (55 mL) was added Pd/C (10% purity, 1.0 equiv) The suspension was degassed under vacuum and purged with H₂ several times. The mixture was stirred under H2 (15 psi) at 25° C. for 1 h. The reaction mixture was Filtrate and concentrated. The residue was used for next step directly. Compound 2-[2-(6-bromohexoxy)ethoxy]ethoxymethanol (3.90 g, 13.0 mmol, 85% yield) was obtained as a yellow oil.

Step 3. A mixture of 2-[2-(5-bromopentoxy)ethoxy]ethanol (1.48 g, 5.79 mmol, 2.0 equiv), KI (2.40 g, 14.5 mmol, 5.0 equiv) in DMF (8 mL) was stirred at 80° C. for 4 h. then cooled to 25° C. and 5-fluoro-6-oxo-1H-pyridine-2-carbonitrile (400 mg, 2.90 mmol, 1.0 equiv) and K₂CO₃ (1.20 g, 8.69 mmol, 3.0 equiv) was added. The mixture was stirred at 50° C. for 12 h under N₂ atmosphere. The reaction mixture was quenched by addition water 50 mL at 25° C., and then diluted with Ethyl acetate 50 mL and extracted with Ethyl acetate (50 mL×3). The combined organic layers were washed with brine 50 mL, dried over Na₂SO₄, filtered and concentrated under reduced pressure to give a residue. The residue was purified by column chromatography (SiO₂, Petroleum ether/Ethyl acetate=1/1 to 0/1). Compound 5-fluoro-1-[5-[2-(2-hydroxyethoxy)ethoxy]pentyl]-6-oxo-pyridine-2-carbonitrile (80.0 mg, 256 μmol, 9% yield) was obtained as a white solid. ¹H NMR: (400 MHZ, CHLOROFORM-d) δ=7.09 (t, J=7.9 Hz, 1H), 6.78-6.70 (m, 1H), 4.27-4.19 (m, 2H), 3.76-3.73 (m, 2H), 3.69-3.67 (m, 2H), 3.63-3.59 (m, 4H), 3.52-3.48 (m, 2H), 1.88-1.81 (m, 2H), 1.71-1.66 (m, 2H), 1.55-1.48 (m, 2H). Compound 5-fluoro-6-[5-[2-(2-hydroxyethoxy)ethoxy]pentoxy]pyridine-2-carbonitrile (450 mg, 1.44 mmol, 50% yield) was obtained as a white solid. ¹H NMR: (400 MHz, CHLOROFORM-d) δ=7.42-7.36 (m, 1H), 7.32-7.28 (m, 1H), 4.43 (t, J=6.6 Hz, 2H), 3.75 (br d, J=4.3 Hz, 2H), 3.71-3.68 (m, 2H), 3.65-3.59 (m, 5H), 3.52 (t, J=6.6 Hz, 2H), 1.91-1.82 (m, 2H), 1.72-1.68 (m, 2H), 1.57-1.51 (m, 2H).

Step 4. To a solution of 5-fluoro-1-[5-[2-(2-hydroxyethoxy)ethoxy]pentyl]-6-oxo-pyridine-2-carbonitrile (170 mg, 544 μmol, 1.0 equiv) and Et₃N(291 mg, 2.87 mmol, 400 μL, 5.3 equiv) in DCM (10 mL) was added TosCl (510 mg, 2.68 mmol, 4.9 equiv). The mixture was stirred at 25° C. for 12 h under N₂ atmosphere. The reaction mixture was concentrated under reduced pressure to give a residue. The residue was purified by prep-TLC (SiO2, Petroleum ether: Ethyl acetate OH=1:1). Compound 2-[2-[5-(6-cyano-3-fluoro-2-oxo-1-pyridyl)pentoxy]ethoxy]ethyl 4-methylbenzenesulfonate (250 mg, 536 μmol, 98% yield) was obtained as a white solid.

Step 5. A mixture of 2-[2-[5-(6-cyano-3-fluoro-2-oxo-1-pyridyl)pentoxy]ethoxy]ethyl 4-methylbenzenesulfonate (250 mg, 536 μmol, 1.0 equiv), 5-[[[3-ethyl-5-[(2S)-2-(2-hydroxyethyl)-1-piperidyl]pyrazolo[1,5-a]pyrimidin-7-yl]amino]methyl]pyridin-2-ol (320 mg, 807 μmol, 1.5 equiv) in NMP (2 mL) was added K₂CO₃ (220 mg, 1.59 mmol, 3.0 equiv) and the mixture was stirred at 50° C. for 12 h under N₂ atmosphere. The reaction mixture was poured into water 50 mL at 0° C., and then diluted with Ethyl acetate 50 mL and extracted with Ethyl acetate (50 mL×3). The combined organic layers were washed with brine 10 mL, dried over Na₂SO₄, filtered and concentrated under reduced pressure to give a residue. The residue was purified by prep-TLC (SiO2, DCM: MeOH=15:1). Compound 1-[5-[2-[2-[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]pentyl]-5-fluoro-6-oxo-pyridine-2-carbonitrile (70.0 mg, 101 μmol, 19% yield) was obtained as a white solid. ¹H NMR: (400 MHZ, DMSO-d₆) δ=8.23 (d, J=2.0 Hz, 1H), 7.89-7.81 (m, 1H), 7.80-7.74 (m, 1H), 7.65 (s, 1H), 7.55-7.49 (m, 1H), 7.22-7.16 (m, 1H), 6.79 (d, J=8.8 Hz, 1H), 5.60 (s, 1H), 4.72 (t, J=4.8 Hz, 1H), 4.57 (br d, J=2.0 Hz, 1H), 4.47 (br d, J=5.6 Hz, 2H), 4.36-4.30 (m, 2H), 4.10-3.98 (m, 3H), 3.75-3.67 (m, 2H), 3.57-3.53 (m, 2H), 3.49-3.44 (m, 2H), 3.37 (br t, J=6.4 Hz, 3H), 2.88-2.77 (m, 1H), 1.86-1.76 (m, 1H), 1.71-1.47 (m, 11H), 1.40-1.30 (m, 3H), 1.19-1.15 (m, 4H).

Step 6. A mixture of 1-[5-[2-[2-[[5-[[[3-ethyl-5-[(2S)-2-(2-hydroxyethyl)-1-piperidyl]pyrazolo[1,5-a]pyrimidin-7-yl]amino methyl]-2-pyridyl]oxy]ethoxy]ethoxy]pentyl]-5-fluoro-6-oxo-pyridine-2-carbonitrile (70.0 mg, 101 μmol, 1.0 equiv),3-[(1S)-1-aminoethyl]-6-chloro-1H-quinolin-2-one (130 mg, 502 μmol, 5.0 equiv, HCl) in DMSO (2 mL) was added DIEA (148 mg, 1.15 mmol, 200 μL, 11.0 equiv) and the mixture was stirred at 100° C. for 12 h under N₂ atmosphere. The reaction mixture was concentrated under reduced pressure to give a residue. The residue was purified by prep-HPLC (FA condition; column: column: Unisil 3-100 C18 Ultra 150*50 mm*3 μm; mobile phase: [water (FA)-ACN]; B %: 34%-64%,7 min). Compound 5-[[(1S)-1-(6-chloro-2-oxo-1H-quinolin-3-yl) ethyl]amino]-1-[5-[2-[2-[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]pentyl]-6-oxo-pyridine-2-carbonitrile (20.4 mg, 22.0 μmol, 22% yield, 96% purity) was obtained as a yellow solid. ¹H NMR: (400 MHZ, METHANOL-d₄)8=8.18 (d, J=2.0 Hz, 1H), 7.79-7.73 (m, 2H), 7.60-7.56 (m, 2H), 7.47-7.43 (m, 1H), 7.29 (d, J=8.8 Hz, 1H), 6.81-6.77 (m, 2H), 6.01 (d, J=8.0 Hz, 1H), 5.51 (s, 1H), 4.53 (s, 2H), 4.41-4.37 (m, 2H), 4.12 (br t, J=7.6 Hz, 2H), 4.04-3.97 (m, 1H), 3.83-3.79 (m, 2H), 3.68-3.63 (mz, 2H), 3.59-3.55 (m, 2H), 3.54-3.50 (m, 1H), 3.46 (t, J=6.4 Hz, 2H), 3.42-3.34 (m, 2H), 3.03-2.94 (m, 1H), 2.57-2.49 (m, 2H), 2.12-2.02 (m, 1H), 1.79-1.60 (m, 9H), 1.60-1.54 (m, 5H), 1.53-1.47 (m, 1H), 1.46-1.39 (m, 2H), 1.20 (t, J=7.6 Hz, 3H). LC-MS: MS (ES⁺): RT=2.226 min, m/z=893.6 [M+H⁺], LCMS method 10.

### Example 71—the Synthetic Route for I-92 and I-93

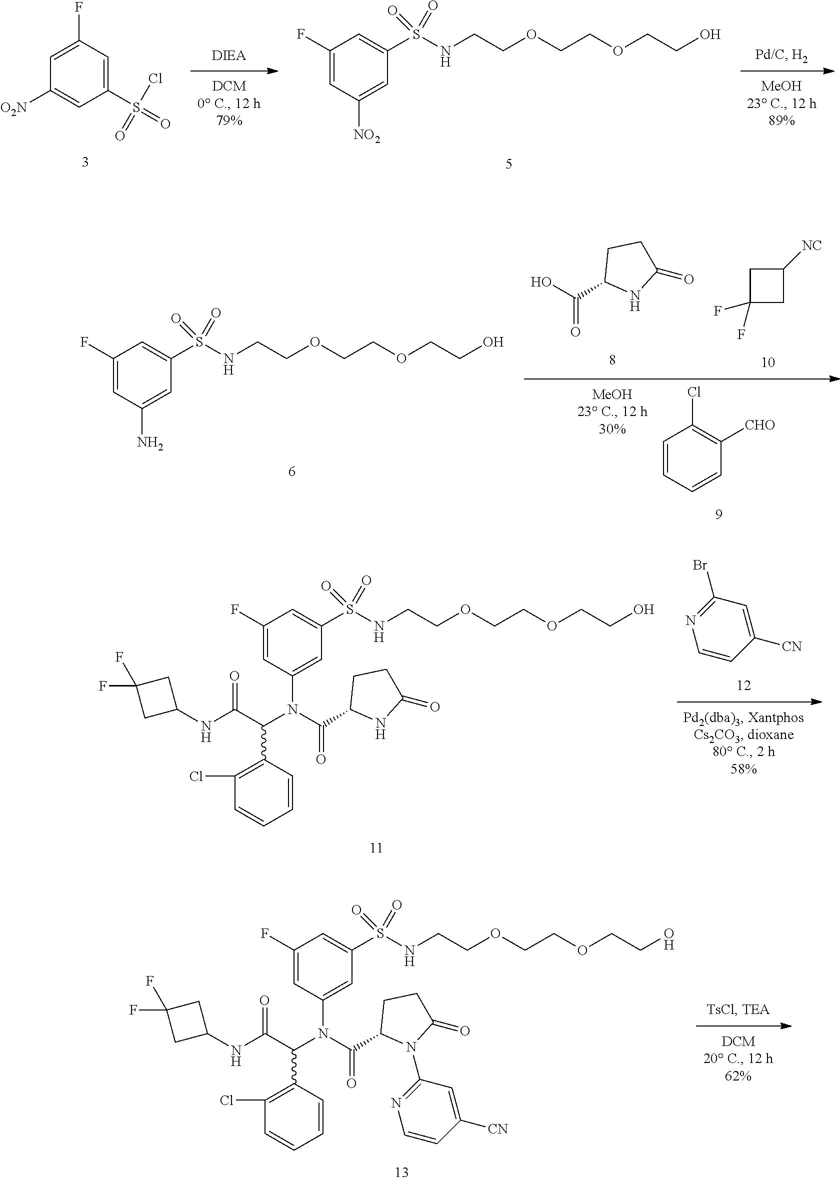

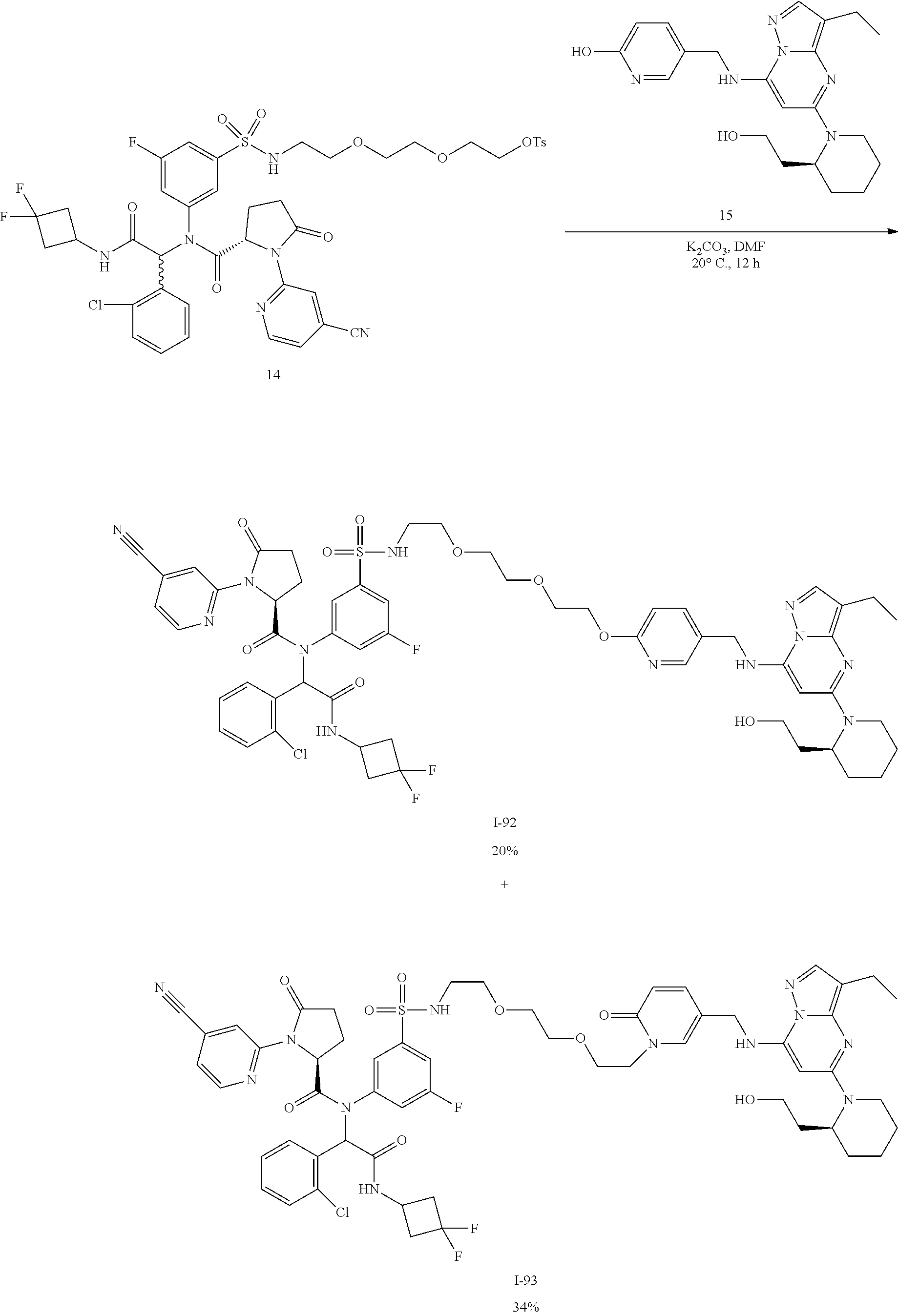

General Information: Synthetic route for compound 3 is described in WO2021/26099. Synthetic route for compound 10 is described in WO2013/107405.

Step 1. A mixture of 3-fluoro-5-nitro-benzenesulfonyl chloride (1.82 g, 7.60 mmol, 1.2 equiv), 2-[2-(2-aminoethoxy)ethoxy]ethanol (916 mg, 6.14 mmol, 1.0 equiv), DIEA (1.20 g, 9.26 mmol, 1.61 mL, 1.5 equiv) in DCM (20 mL) was degassed and purged with N₂ for 3 times, and then the mixture was stirred at 0° C. for 12 h under N₂ atmosphere. The reaction mixture was diluted with water (30 mL) and extracted with DCM (30 mL×3). The combined organic layers were washed with brine (10 mL×3), dried over Na₂SO₄, filtered and concentrated under reduced pressure to give a residue. The residue was purified by column chromatography (SiO₂, Petroleum ether/Ethyl acetate=50/1 to 10/1). Compound 3-fluoro-N-[2-[2-(2-hydroxyethoxy)ethoxy]ethyl]-5-nitro-benzenesulfonamide (1.71 g, 4.85 mmol, 79% yield) was obtained as a yellow oil.

Step 2. A mixture of 3-fluoro-N-[2-[2-(2-hydroxyethoxy)ethoxy]ethyl]-5-nitro-benzenesulfonamide (1.71 g, 4.85 mmol, 1.0 equiv), Pd/C (500 mg, 10% purity), H2 (9.8 mg, 4.8 mmol, 1.0 equiv) in MeOH (20 mL) was degassed and purged with H₂ for 3 times, and then the mixture was stirred at 23° C. for 12 h under H₂ atmosphere. The mixture filtered and concentrated under reduced pressure to give a residue. The mixture was used for next step without purification. Compound 3-amino-5-fluoro-N-[2-[2-(2-hydroxyethoxy)ethoxy]ethyl]benzenesulfonamide (1.40 g, 4.33 mmol, 89% yield) was obtained as a brown oil.

Step 3. A mixture of 3-amino-5-fluoro-N-[2-[2-(2-hydroxyethoxy)ethoxy]ethyl]benzenesulfonamide (1.3 g, 4.0 mmol, 1.0 equiv), 2-chlorobenzaldehyde (567 mg, 4.03 mmol, 454 μL, 1.0 equiv) in MeOH (15 mL) was stirred at 23° C. for 0.5 h. Then (2S)-5-oxopyrrolidine-2-carboxylic acid (520 mg, 4.03 mmol, 1.0 equiv) and 1,1-difluoro-3-isocyano-cyclobutane (1.42 g, 12.1 mmol, 3.0 equiv) was added. The mixture was stirred at 23° C. for 11.5 h under N₂ atmosphere. The mixture was filtered and concentrated to give the residue. The residue was purified by prep-HPLC (FA condition; column: YMC Triart C18 250*50 mm*7 μm; mobile phase: [water (NH₄HCO₃)-ACN]; B %: 5%-35%,30 min). Compound (2S)—N-[1-(2-chlorophenyl)-2-[(3,3-difluorocyclobutyl)amino]-2-oxo-ethyl]-N-[3-fluoro-5-[2-[2-(2-hydroxyethoxy)ethoxy]ethylsulfamoyl]phenyl]-5-oxo-pyrrolidine-2-carboxamide (842 mg, 1.22 mmol, 30% yield) was obtained as a white solid.

Step 4. A mixture of 2-bromopyridine-4-carbonitrile (79.4 mg, 434 μmol, 1.0 equiv), (2S)—N-[1-(2-chlorophenyl)-2-[(3,3-difluorocyclobutyl)amino]-2-oxo-ethyl]-N-[3-fluoro-5-[2-[2-(2-hydroxyethoxy)ethoxy]ethylsulfamoyl]phenyl]-5-oxo-pyrrolidine-2-carboxamide (300 mg, 434 μmol, 1.0 equiv), Pd₂ (dba)3 (39.8 mg, 43.4 μmol, 0.1 equiv), Xantphos (25.1 mg, 43.4 μmol, 0.1 equiv), Cs₂CO₃ (212 mg, 651 μmol, 1.5 equiv) in dioxane (20 mL) was degassed with N₂ and stirred under N₂ at 80° C. for 2 h. The mixture was filtered and concentrated to give a residue. The residue was purified by prep-HPLC (column: Waters xbridge 150*25 mm*10 μm; mobile phase: [water (NH₄HCO₃)-ACN]; B %: 29%-59%,8 min) to give the desired product (2S)—N-[1-(2-chlorophenyl)-2-[(3,3-difluorocyclobutyl)amino]-2-oxo-ethyl]-1-(4-cyano-2-pyridyl)-N-[3-fluoro-5-[2-[2-(2-hydroxyethoxy)ethoxy]ethylsulfamoyl]phenyl]-5-oxo-pyrrolidine-2-carboxamide (200 mg, 252 μmol, 58% yield) as a yellow solid.

Step 5. A mixture of (2S)—N-[1-(2-chlorophenyl)-2-[(3,3-difluorocyclobutyl)amino]-2-oxo-ethyl]-1-(4-cyano-2-pyridyl)-N-[3-fluoro-5-[2-[2-(2-hydroxyethoxy)ethoxy]ethylsulfamoyl]phenyl]-5-oxo-pyrrolidine-2-carboxamide (90.0 mg, 113 μmol, 1.0 equiv), TosCl (64.9 mg, 340 μmol, 3.0 equiv), TEA (45.9 mg, 454 μmol, 63.2 μL, 4.0 equiv) in DCM (2 mL) was stirred under N₂ at 20° C. for 12 h. The mixture was concentrated to give a residue. The residue was purified by prep-TLC (Petroleum ether: Ethyl acetate=2:3) to give the desired product 2-[2-[2-[3-[[1-(2-chlorophenyl)-2-[(3,3-difluorocyclobutyl)amino]-2-oxo-ethyl]-[(2S)-1-(4-cyano-2-pyridyl)-5-oxo-pyrrolidine-2-carbonyl]amino]-5-fluoro-phenyl]sulfonylamino]ethoxy]ethoxy]ethyl 4-methylbenzenesulfonate (70 mg, 70.0 μmol, 62% yield, 94.770% purity) as a white solid.

Step 6. A mixture of 2-[2-[2-[[3-[1-(2-chlorophenyl)-2-[(3,3-difluorocyclobutyl) amino]-2-oxo-ethyl]-[(2S)-1-(4-cyano-2-pyridyl)-5-oxo-pyrrolidine-2-carbonyl]amino]-5-fluoro-phenyl]sulfonylamino]ethoxy]ethoxy]ethyl 4-methylbenzenesulfonate (60 mg, 63 μmol, 1.0 equiv), 5-[[3-ethyl-5-[(2S)-2-(2-hydroxyethyl)-1-piperidyl]pyrazolo[1,5-a]pyrimidin-7-yl]amino methyl]pyridin-2-ol (176 mg, 443 μmol, 7.0 equiv), K₂CO₃ (43.8 mg, 317 μmol, 5.0 equiv) in DMF (1 mL) was stirred under N₂ at 20° C. for 12 h. The mixture diluted with EA (60 mL). The solution was washed with brine (10 mL×4), dried, filtered and concentrated to give a residue. The residue was purified by prep-TLC (Dichloromethane: Methanol=10:1) to give two crude desired product. The two desired product was further purified by prep-HPLC (FA) to give (2S)—N-[1-(2-chlorophenyl)-2-[(3,3-difluorocyclobutyl)amino]-2-oxo-ethyl]-1-(4-cyano-2-pyridyl)-N-[3-[2-[2-[2-[5-[[3-ethyl-5-[(2S)-2-(2-hydroxyethyl)-1-piperidyl]pyrazolo[1,5-a]pyrimidin-7-yl]amino methyl]-2-pyridyl]oxy]ethoxy]ethoxy]ethylsulfamoyl]-5-fluoro-phenyl]-5-oxo-pyrrolidine-2-carboxamide (14.82 mg, 12.58 μmol, 19.86% yield, 99.456% purity) as a yellow solid and (2S)—N-[1-(2-chlorophenyl)-2-[(3,3-difluorocyclobutyl)amino]-2-oxo-ethyl]-1-(4-cyano-2-pyridyl)-N-[3-[2-[2-[2-[5-[ [[3-ethyl-5-[(2S)-2-(2-hydroxyethyl)-1-piperidyl]pyrazolo[1,5-a]pyrimidin-7-yl]amino]methyl]-2-oxo-1-pyridyl]ethoxy]ethoxy]ethylsulfamoyl]-5-fluoro-phenyl]-5-oxo-pyrrolidine-2-carboxamide (25.91 mg, 21.72 μmol, 34.30% yield, 98.222% purity) as a white solid. Spectra for I-92: 1H NMR: (400 MHZ, CD₃OD) δ=8.68-8.59 (m, 2H), 8.19 (m, 1H), 7.87 (m, 1H), 7.75-7.73 (m, 1H), 7.63 (m, 1H), 7.43-7.41 (m, 3H), 7.25 (m, 1H), 7.08 (m, 1H), 6.99 (m, 1H), 6.81-6.79 (m, 1H), 6.53-6.46 (m, 1H), 5.52 (s, 1H), 4.54 (m, 3H), 4.41 (m, 2H), 4.20 (m, 2H), 3.82-3.81 (m, 2H), 3.63-3.62 (m, 6H), 3.52-3.50 (m, 3H), 2.99 (m, 6H), 2.56 (m, 6H), 2.10 (m, 3H), 1.73-1.64 (m, 7H), 1.24-1.21 (t, J=7.6 Hz, 3H). LC-MS: MS (ES⁺): RT=2.885 min, m/z=1171.6 [M+H +] LCMS method 100.

Spectra for I-93: 1H NMR: (400 MHZ, CD₃OD) δ=8.79-8.54 (m, 2H), 7.87-7.77 (m, 2H), 7.63-7.61 (m, 2H), 7.46-7.37 (m, 3H), 7.25 (m, 1H), 7.08 (m, 1H), 6.99 (m, 1H), 6.57-6.52 (m, 2H), 5.57-5.54 (m, 1H), 4.41-4.39 (m, 2H), 4.16-4.15 (m, 4H), 3.75 (m, 2H), 3.47 (m, 6H), 3.31 (m, 2H), 2.95 (m, 6H), 2.56-2.52 (m, 6H), 2.10 (m, 3H), 1.69-1.66 (m, 7H), 1.24-1.20 (m, 3H). LC-MS: MS (ES⁺): RT=2.821 min, m/z=1171.6 [M+H⁺] LCMS method 100.

### Example 72—the Synthetic Route for I-94

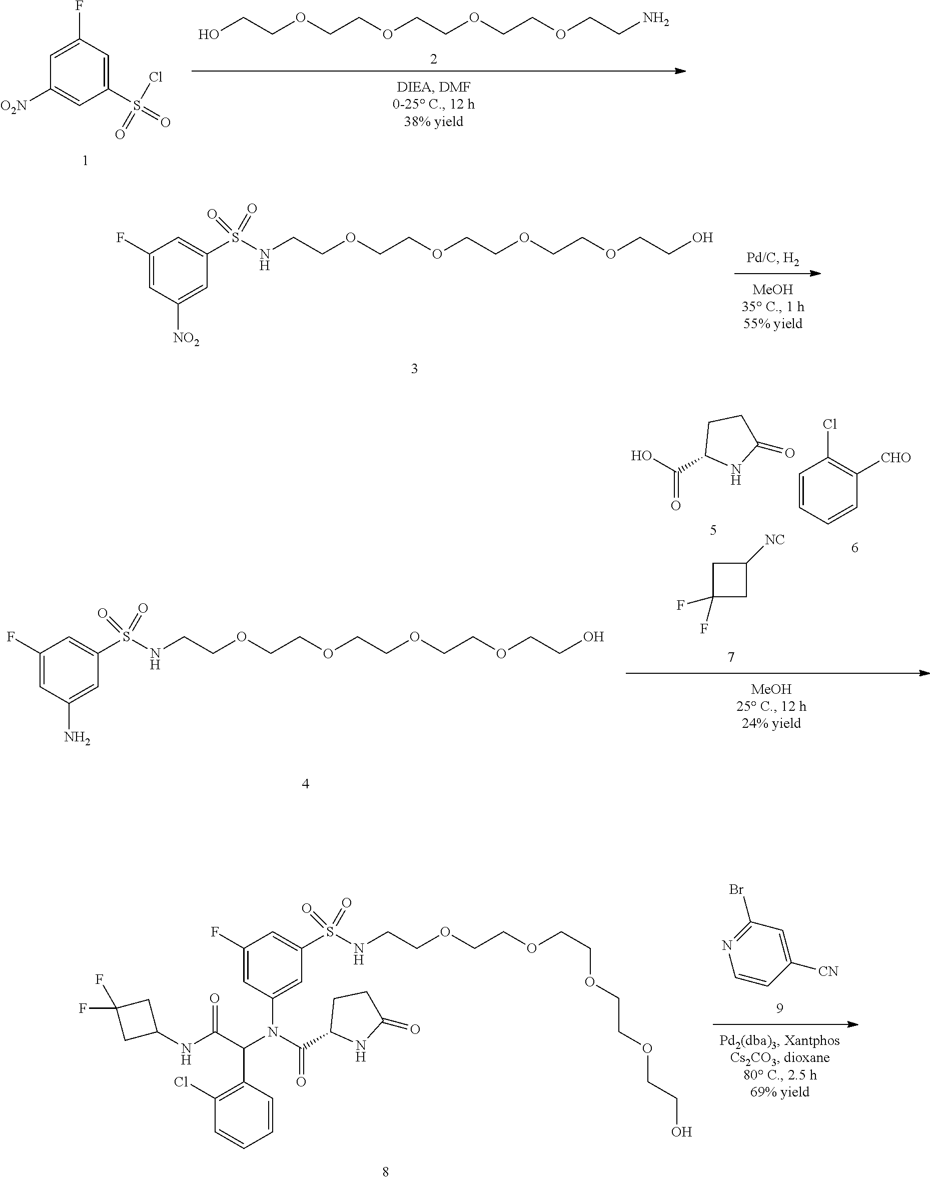

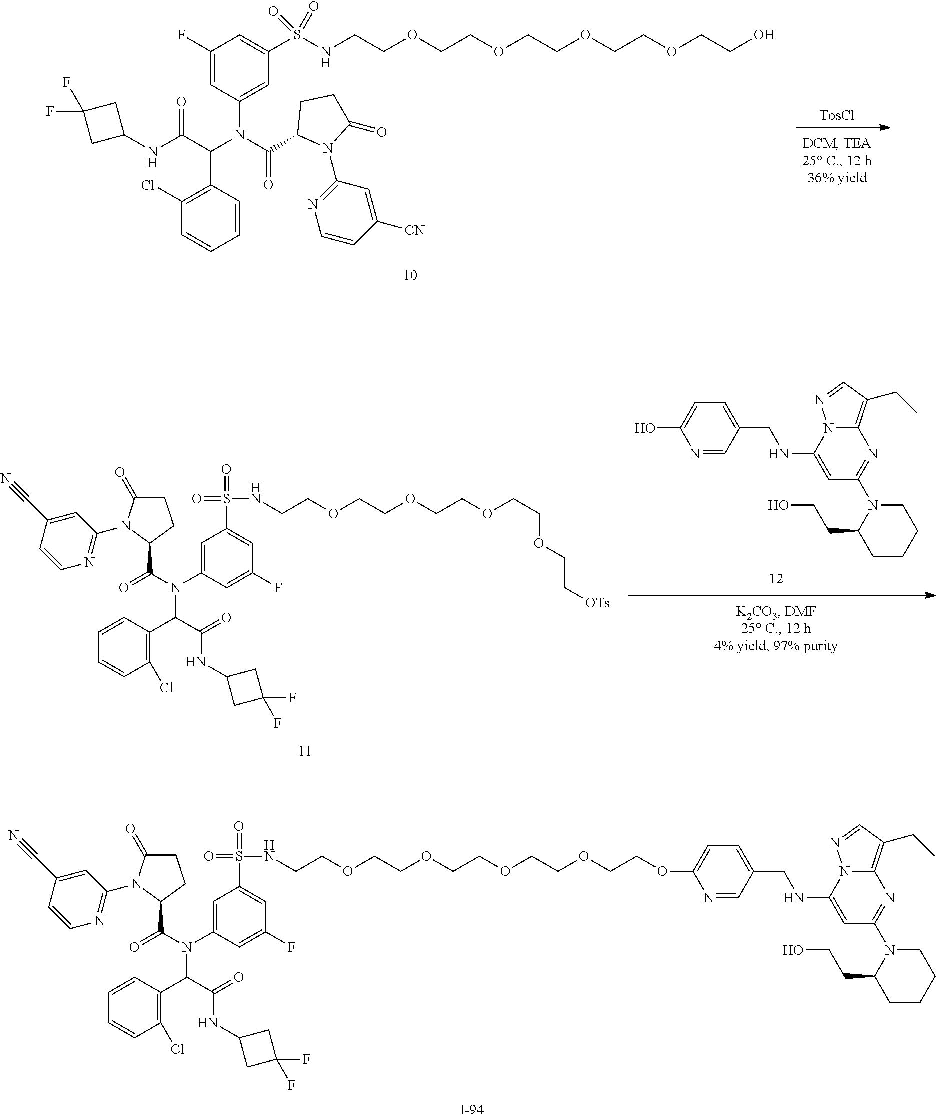

General Information: Synthetic route for compound 7 is described in WO2013/107405.

Step 1. To a solution of 2-[2-[2-[2-(2-aminoethoxy)ethoxy]ethoxy]ethoxy]ethanol (594 mg, 2.5 mmol, 1.2 equiv) in DCM (5.0 mL) was added DIEA (404 mg, 3.1 mmol, 545 μL, 1.5 equiv) and 3-fluoro-5-nitro-benzenesulfonyl chloride (500 mg, 2.1 mmol, 1.0 equiv) in 0° C. Then, the mixture was stirred at 0-25° C. for 12 h. The reaction mixture was filtered. The residue was purified by prep-HPLC (column: Phenomenex luna C18 150*25 mm*10 μm; mobile phase: [water (FA)-ACN]; B %: 20%-50%, 10 min) to give the 3-fluoro-N-[2-[2-[2-[2-(2-hydroxyethoxy)ethoxy]ethoxy]ethoxy]ethyl]-5-nitro-benzenesulfonamide (350 mg, 795 μmol, 38% yield) as a colorless oil. ¹H NMR: (400 MHZ, CDCl₃) δ=8.60 (s, 1H), 8.13-8.07 (m, 1H), 8.05-8.00 (m, 1H), 7.72-7.65 (m, 1H), 3.82-3.78 (m, 2H), 3.73-3.64 (m, 11H), 3.61-3.58 (m, 2H), 3.55-3.50 (m, 4H), 3.24-3.19 (m, 2H).

Step 2. A mixture of benzyl 3-fluoro-N-[2-[2-[2-[2-(2-hydroxyethoxy)ethoxy]ethoxy]ethoxy]ethyl]-5-nitro-benzenesulfonamide (1.3 g, 3.0 mmol, 1.0 equiv), Pd/C (1.0 g, 3.0 mmol, 10% purity, 1.0 equiv) in MeOH (17 mL) was degassed and purged with H₂ for 3 times, and then the mixture was stirred at 35° C. for 1 h under H₂ atmosphere. The mixture was filtered and concentrated to get the residue. The residue was purified by flash silica gel chromatography (ISCO®; 2 g SepaFlash® Silica Flash Column, Eluent of 100% Ethyl acetate/Petroleum ethergradient at 10 mL/min) to give the 3-amino-5-fluoro-N-[2-[2-[2-[2-(2-hydroxyethoxy) ethoxy]ethoxy]ethoxy]ethyl]benzenesulfonamide (700 mg, 1.7 mmol, 55% yield) as a yellow oil.

Step 3. To a solution of 3-amino-5-fluoro-N-[2-[2-[2-[2-(2-hydroxyethoxy)ethoxy]ethoxy]ethoxy]ethyl]benzenesulfonamide (700 mg, 1.7 mmol, 1.0 equiv), 2-chlorobenzaldehyde (263 mg, 1.9 mmol, 210 μL, 1.1 equiv) in MeOH (10 mL). The mixture was stirred at 25° C. for 30 min. Then added (2S)-5-oxopyrrolidine-2-carboxylic acid (242 mg, 1.9 mmol, 1.1 equiv) and was stirred at 25° C. for 10 min. Then added 1,1-difluoro-3-isocyano-cyclobutane (399 mg, 3.4 mmol, 2.0 equiv). The mixture was stirred at 25° C. for 12 h. The reaction mixture was filtered. The residue was purified by prep-HPLC (column: Phenomenex luna C18 150*25 mm*10 μm; mobile phase: [water (FA)-ACN]; B %: 25%-55%, 10 min) to give the (2S)—N-[1-(2-chlorophenyl)-2-[(3,3-difluorocyclobutyl)amino]-2-oxo-ethyl]-N-[3-fluoro-5-[2-[2-[2-[2-(2-hydroxyethoxy)ethoxy]ethoxy]ethoxy]ethylsulfamoyl]phenyl]-5-oxo-pyrrolidine-2-carboxamide (328 mg, 420 μmol, 24% yield) as a black brown solid. ¹H NMR: (400 MHZ, DMSO-d₆) δ=8.95 (s, 1H), 8.09-7.72 (m, 4H), 7.51-7.39 (m, 3H), 7.29-7.17 (m, 2H), 7.10 (d, J=7.2 Hz, 2H), 6.99-6.73 (m, 2H), 6.40-6.29 (m, 1H), 4.69-4.47 (m, 1H), 4.22-4.03 (m, 2H), 4.01-3.82 (m, 1H), 3.50-3.39 (m, 20H), 2.18-1.68 (m, 6H).

Step 4. A mixture of (2S)—N-[1-(2-chlorophenyl)-2-[(3,3-difluorocyclobutyl)amino]-2-oxo-ethyl]-N-[3-fluoro-5-[2-[2-[2-[2-(2-hydroxyethoxy)ethoxy]ethoxy]ethoxy]ethylsulfamoyl]phenyl]-5-oxo-pyrrolidine-2-carboxamide (278 mg, 356 μmol, 1.0 equiv), 2-bromopyridine-4-carbonitrile (65 mg, 356 μmol, 1.0 equiv), Cs₂CO₃ (174 mg, 535 μmol, 1.5 equiv), Pd₂ (dba)3 (32 mg, 35 μmol, 0.1 equiv) and Xantphos (20 mg, 35 μmol, 0.1 equiv) in dioxane (12 mL) was degassed and purged with N₂ for 3 times, and then the mixture was stirred at 80° C. for 2.5 h under N₂ atmosphere. The reaction mixture was filtered and concentrated under reduced pressure to give a residue. The residue was purified by flash silica gel chromatography (ISCO®; 1 g SepaFlash® Silica Flash Column, Eluent of 0˜100% Ethyl acetate/Petroleum ethergradient at 18 mL/min) to give the (2S)—N-[1-(2-chlorophenyl)-2-[(3,3-difluorocyclobutyl)amino]-2-oxo-ethyl]-1-(4-cyano-2-pyridyl)-N-[3-fluoro-5-[2-[2-[2-[2-(2-hydroxyethoxy)ethoxy]ethoxy]ethoxy]ethylsulfamoyl]phenyl]-5-oxo-pyrrolidine-2-carboxamide (220 mg, 249 μmol, 69% yield) as a yellow oil.

Step 5. A mixture of (2S)—N-[1-(2-chlorophenyl)-2-[(3,3-difluorocyclobutyl)amino]-2-oxo-ethyl]-1-(4-cyano-2-pyridyl)-N-[3-fluoro-5-[2-[2-[2-[2-(2-hydroxyethoxy)ethoxy]ethoxy]ethoxy]ethylsulfamoyl]phenyl]-5-oxo-pyrrolidine-2-carboxamide (200 mg, 226 μmol, 1.0 equiv), TEA (229 mg, 2.27 mmol, 315 μL, 10 equiv) in DCM (2 mL) was degassed and purged with N₂ for 3 times, then added TosCl (64 mg, 340 μmol, 1.5 equiv) and then the mixture was stirred at 25° C. for 12 h under N₂ atmosphere. The reaction mixture was filtered. The residue was purified by prep-TLC (SiO2, Petroleum ether: Ethyl acetate=1:3) to give the 2-[2-[2-[2-[2-[3-[[1-(2-chlorophenyl)-2-[(3,3-difluorocyclobutyl)amino]-2-oxo-ethyl]-[(2S)-1-(4-cyano-2-pyridyl)-5-oxo-pyrrolidine-2-carbonyl]amino]-5-fluoro-phenyl]sulfonylamino]ethoxy]ethoxy]ethoxy]ethoxy]ethyl 4-methylbenzenesulfonate (85 mg, 82 μmol, 36% yield) as a yellow solid.

Step 6. A mixture of 2-[2-[2-[2-[2-[[3-[[1-(2-chlorophenyl)-2-[(3,3-difluorocyclo butyl)amino]-2-oxo-ethyl]-[(2S)-1-(4-cyano-2-pyridyl)-5-oxo-pyrrolidine-2-carbonyl]amino]-5-fluoro-phenyl]sulfonylamino]ethoxy]ethoxy]ethoxy]ethoxy]ethyl 4-methylbenzenesulfonate (85 mg, 82 μmol, 1.0 equiv), 5-[[3-ethyl-5-[(2S)-2-(2-hydroxyethyl)-1-piperidyl]pyrazolo[1,5-a]pyrimidin-7-yl]amino methyl]pyridin-2-ol (227 mg, 574 μmol, 7.0 equiv), K₂CO₃ (79 mg, 574 μmol, 7.0 equiv) in DMF (1 mL) was degassed and purged with N₂ for 3 times, and then the mixture was stirred at 25° C. for 12 h under N₂ atmosphere. The reaction mixture was quenched by addition EA (60 mL) at 25° C. The combined organic layers were washed with brine (4×10 mL), dried over [Na₂SO₄], filtered and concentrated under reduced pressure to give a residue. The residue was purified by prep-TLC (SiO2, DCM: MeOH=10:1) and prep-HPLC (column: Phenomenex luna C18 150*25 mm*10 μm; mobile phase: [water (FA)-ACN]; B %: 32%-62%,10 min), and purified by prep-HPLC (column: Phenomenex luna C18 150*25 mm*10 μm; mobile phase: [water (FA)-ACN]; B %: 30%-60%, 10 min) to give the (2S)—N-[1-(2-chlorophenyl)-2-[(3,3-difluorocyclobutyl)amino]-2-oxo-ethyl]-1-(4-cyano-2-pyridyl)-N-[3-[2-[2-[2-[2-[2-[5-[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]ethylsulfamoyl]-5-fluoro-phenyl]-5-oxo-pyrrolidine-2-carboxamide (4 mg, 3 μmol, 4% yield, 97% purity) as a white solid. ¹H NMR: (400 MHZ, MeOD) δ=8.83-8.57 (m, 2H), 8.29-8.17 (m, 1H), 7.99-7.85 (m, 1H), 7.78-7.74 (m, 1H), 7.62 (s, 1H), 7.49-7.36 (m, 3H), 7.26-7.17 (m, 1H), 7.12-7.08 (m, 1H), 7.01-6.97 (m, 1H), 6.82-6.78 (m, 1H), 6.56-6.39 (m, 1H), 5.53 (s, 1H), 4.59-4.52 (m, 3H), 4.42-4.37 (m, 2H), 4.33-3.95 (m, 3H), 3.85-3.77 (m, 2H), 3.68-3.52 (m, 11H), 3.50-3.33 (m, 7H), 3.05-2.65 (m, 7H), 2.63-2.32 (m, 6H), 2.25-2.03 (m, 3H), 1.76-1.46 (m, 7H), 1.27-1.17 (m, 3H). LC-MS: MS (ES⁺): RT=2.917 min, m/z=1259.8 [M+H⁺] LCMS method 100.

### Example 73—the Synthetic Route for I-95

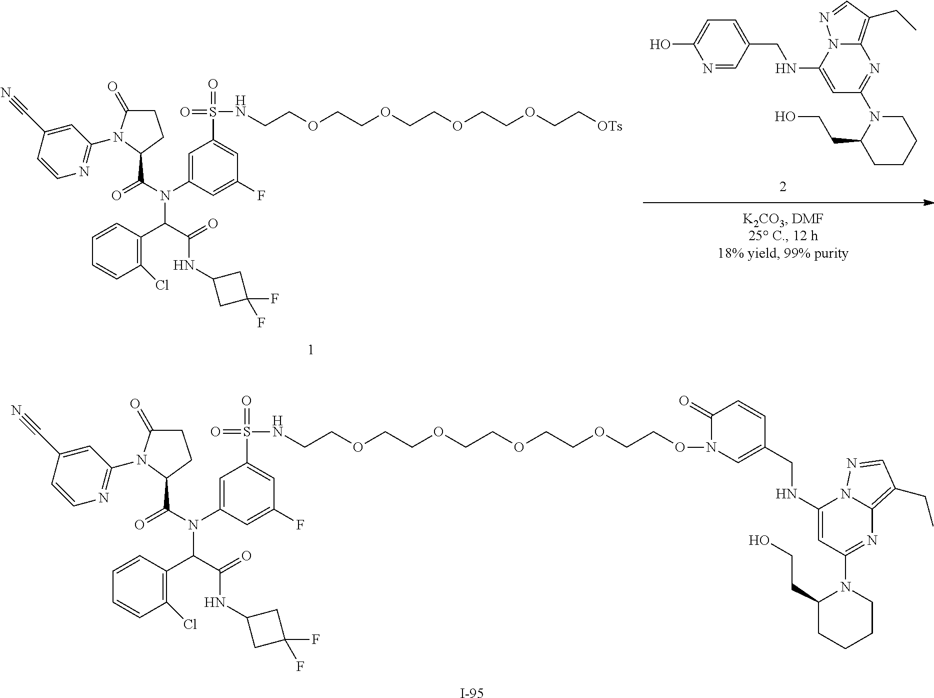

Step 1. A mixture of 2-[2-[2-[2-[2-[[3-[[1-(2-chlorophenyl)-2-[(3,3-difluorocyclobutyl)amino]-2-oxo-ethyl]-[(2S)-1-(4-cyano-2-pyridyl)-5-oxo-pyrrolidine-2-carbonyl]amino]-5-fluoro-phenyl]sulfonylamino]ethoxy]ethoxy]ethoxy]ethoxy]ethyl 4-methylbenzenesulfonate (85 mg, 82 μmol, 1.0 equiv), 5-[[[3-ethyl-5-[(2S)-2-(2-hydroxyethyl)-1-piperidyl]pyrazolo[1,5-a]pyrimidin-7-yl]amino]methyl]pyridin-2-ol (227 mg, 574 μmol, 7.0 equiv), K₂CO₃ (79 mg, 574 μmol, 7.0 equiv) in DMF (1 mL) was degassed and purged with N₂ for 3 times, and then the mixture was stirred at 25° C. for 12 h under N₂ atmosphere. The reaction mixture was quenched by addition EA (60 mL) at 25° C. The combined organic layers were washed with brine (4×10 mL), dried over Na₂SO₄, filtered and concentrated under reduced pressure to give a residue. The residue was purified by prep-TLC (SiO2, DCM: MeOH =10:1) and prep-HPLC (column: Phenomenex luna C18 150*25 mm*10 μm; mobile phase: [water (FA)-ACN]; B %: 32%-62%, 10 min), and purified by prep-HPLC (column: Phenomenex luna C18 150*25 mm*10 μm; mobile phase: [water (FA)-ACN]; B %: 30% -60% ,10 min) to give the (2S)—N-[1-(2-chlorophenyl)-2-[(3,3-difluorocyclobutyl)amino]-2-oxo-ethyl]-1-(4-cyano-2-pyridyl)-N-[3-[2-[2-[2-[2-[2-[5-[[3-ethyl-5-[(2S)-2-(2-hydroxyethyl)-1-piperidyl]pyrazolo[1,5-a]pyrimidin-7-yl]amino]methyl]-2-oxo-1-pyridyl]ethoxy]ethoxy]ethoxy]ethoxy]ethylsulfamoyl]-5-fluoro-phenyl]-5-oxo-pyrrolidine-2-carboxamide (19 mg, 15 μmol, 18% yield, 99% purity) as a white solid. ¹H NMR: (400 MHZ, MeOD) δ=8.83-8.56 (m, 2H), 8.05-7.83 (m, 1H), 7.74 (s, 1H), 7.66-7.58 (m, 2H), 7.52-7.35 (m, 3H), 7.29-7.16 (m, 1H), 7.13-7.06 (m, 1H), 7.03-6.96 (m, 1H), 6.59-6.38 (m, 2H), 5.57-5.50 (m, 1H), 4.71-4.56 (m, 1H), 4.42-4.35 (m, 2H), 4.29-4.06 (m, 4H), 3.77-3.71 (m, 2H), 3.59-3.37 (m, 16H), 3.35 (d, J=4.2 Hz, 2H), 3.05-2.71 (m, 6H), 2.66-2.33 (m, 6H), 2.23-2.03 (m, 3H), 1.76-1.48 (m, 7H), 1.26-1.17 (m, 3H). LC-MS: MS (ES⁺): RT =2.843 min, m/z=1259.7 [M+H⁺] LCMS Method 100.

### Example 74—the Synthetic Route for I-96

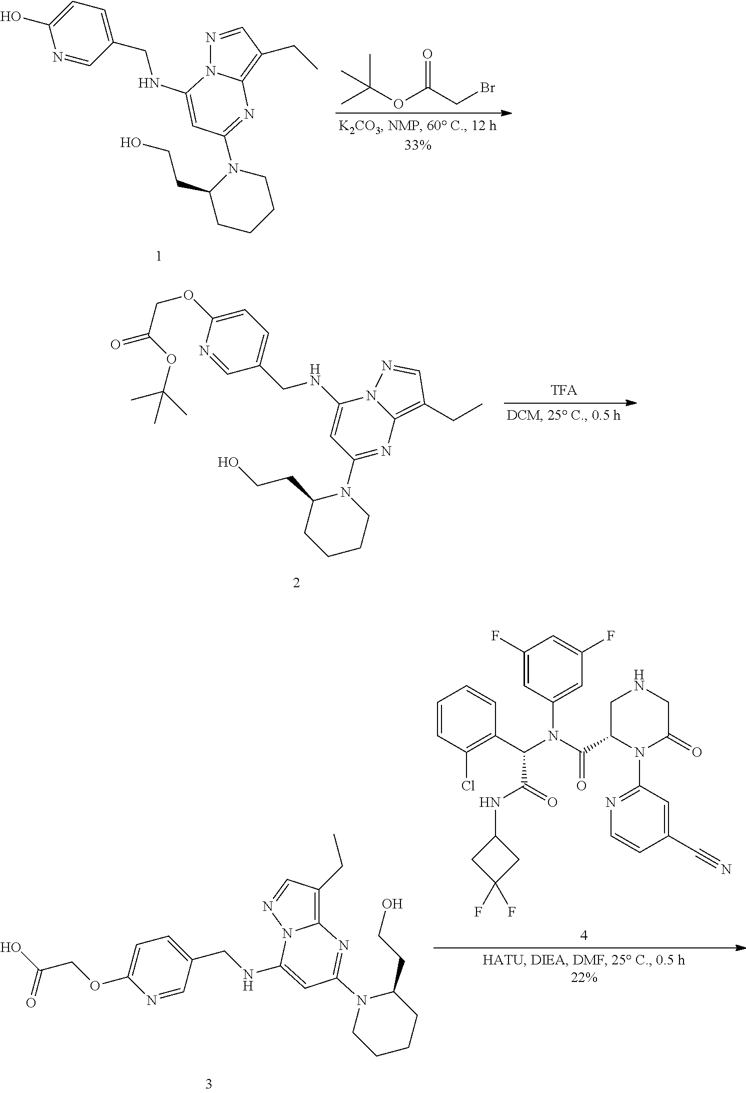

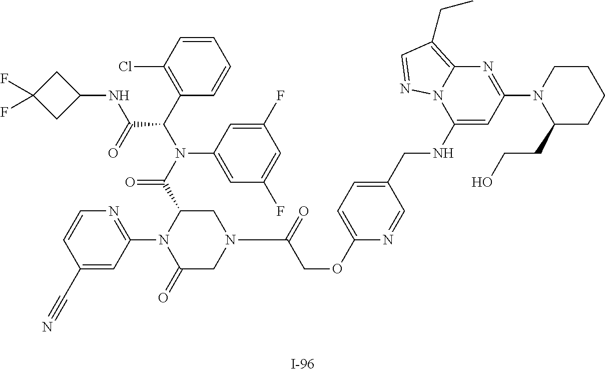

General Information: Synthetic route for compound 4 is described in WO2013/107291.

Step 1. To a solution of 5-[[3-ethyl-5-[(2S)-2-(2-hydroxyethyl)-1-piperidyl]pyrazolo[1,5-a]pyrimidin-7-yl]amino]methyl]pyridin-2-ol (200 mg, 504 μmol, 1.0 equiv) in NMP (2 mL) was added K₂CO₃ (209 mg, 1.5 mmol, 3.0 equiv) and tert-butyl 2-bromoacetate (127 mg, 655 μmol, 1.3 equiv). The mixture was stirred at 60° C. for 12 h. The reaction mixture was filtered and concentrated. The residue was purified by prep-HPLC (column: Phenomenex C18 75*30 mm*3 μm; mobile phase: [water (FA)-ACN]; B %: 22%-52%,7 min) to give tert-butyl 2-[[5-[[[3-ethyl-5-[(2S)-2-(2-hydroxyethyl)-1-piperidyl]pyrazolo[1,5-a]pyrimidin-7-yl]aminomethyl]-2-pyridyl]oxy]acetate (14 mg, 5% yield).

Step 2. To a solution of tert-butyl 2-[[5-[3-ethyl-5-[(2S)-2-(2-hydroxyethyl)-1-piperidyl]pyrazolo[1,5-a]pyrimidin-7-yl]amino]methyl]-2-pyridyl]oxy]acetate (24 mg, 47 μmol, 1.0 equiv) in DCM (1.5 mL) was added TFA (0.5 mL). The mixture was stirred at 25° C. for 0.5 h. The mixture was filtered and concentrated. The residue was used into the next step without further purification.

Step 3. To a solution of (2S)—N-[(1S)-1-(2-chlorophenyl)-2-[(3,3-difluorocyclobutyl) amino]-2-oxo-ethyl]-1-(4-cyano-2-pyridyl)-N-(3,5-difluorophenyl)-6-oxo-piperazine-2-carboxamide (31 mg, 42 μmol, 1.0 equiv, TFA salt) and 2-[5-[[3-ethyl-5-[(2S)-2-(2-hydroxyethyl)-1-piperidyl]pyrazolo[1,5-a]pyrimidin-7-yl]amino]methyl]-2-pyridyl]oxy]acetic acid (26 mg, 45 μmol, 1.0 equiv) in DMF (0.5 mL) was added HATU (24 mg, 63 μmol, 1.5 equiv) and DIEA (37 mg, 287 μmol, 6.7 equiv). The mixture was stirred at 25° C. for 0.5 h and concentrated. The residue was purified by prep-HPLC (column: Phenomenex C18 75*30 mm*3 μm; mobile phase: [water (FA)-ACN]; B %: 30%-60%,7 min) to give (2S)—N-[(1S)-1-(2-chlorophenyl)-2-[(3,3-difluorocyclobutyl)amino]-2-oxo-ethyl]-1-(4-cyano-2-pyridyl)-N-(3,5-difluorophenyl)-4-[2-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]acetyl]-6-oxo-piperazine-2-carboxamide (10 mg, 22% yield). ¹H NMR (400 MHZ, CHLOROFORM-d) δ 8.78-8.70 (m, 1H), 8.58-8.46 (m, 2H), 8.30 (s, 1H), 7.73-7.62 (m, 2H), 7.43-7.36 (m, 3H), 7.24-7.15 (m, 1H), 7.08 (s, 1H), 6.99-6.92 (m, 2H), 6.83-6.67 (m, 3H), 6.59-6.42 (m, 2H), 6.26 (s, 1H), 5.40-5.33 (m, 1H), 5.18-5.06 (m, 3H), 4.98-4.85 (m, 3H), 4.56-4.36 (m, 4H), 4.17-4.10 (m, 1H), 3.81-3.59 (m, 3H), 3.50-3.30 (m, 1H), 3.22-3.04 (m, 3H), 2.99-2.70 (m, 4H), 2.64-2.55 (m, 1H), 2.52-2.44 (m, 2H), 2.15-2.06 (m, 2H), 1.33-1.21 (m, 3H), 1.15 (t, J=7.6 Hz, 3H). LC-MS: MS (ES⁺): RT=2.476 min, m/z=1052.2 [M+H]⁺: LCMS Method 25.

EXAMPLE-75 the Synthetic Route for I-97

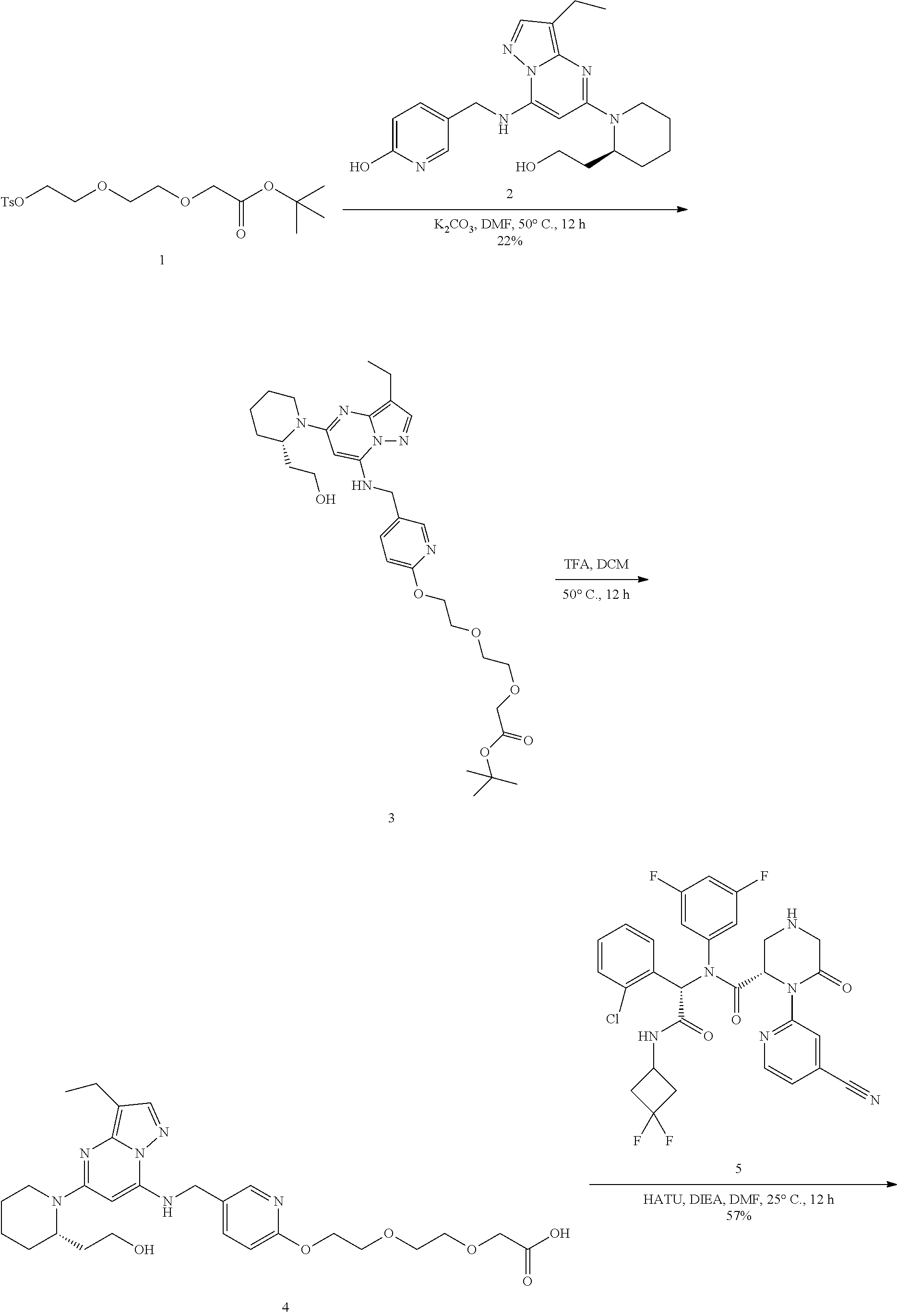 [SMILES: CCc1cnn2c(NCc3ccc(O)nc3)cc(N3CCCC[C@H]3CCO)nc12 ; Cc1ccc(S(=O)(=O)OCCOCCOCC(=O)OC(C)(C)C)cc1 ; CCc1cnn2c(NCc3ccc(OCCOCCOCC(=O)OC(C)(C)C)nc3)cc(N3CCCC[C@H]3CCO)nc12 ; CCc1cnn2c(NCc3ccc(OCCOCCOCC(=O)O)nc3)cc(N3CCCC[C@H]3CCO)nc12 ; N#Cc1ccnc(N2C(=O)CNC[C@H]2C(=O)N(c2cc(F)cc(F)c2)[C@H](C(=O)NC2CC(F)(F)C2)c2ccccc2Cl)c1]

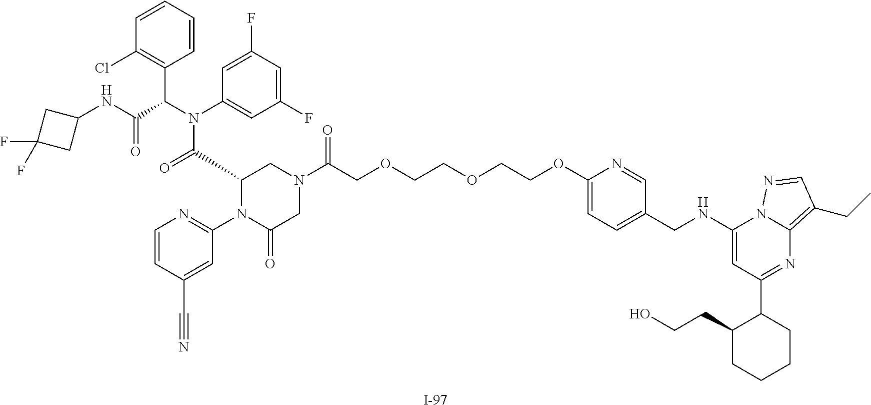

General Information: Compound 1 is described in US2019/300521, 2019, A¹. Compound 5 is described in WO2013/107291.

Step 1. To a solution of tert-butyl 2-[2-[2-(p-tolylsulfonyloxy)ethoxy]ethoxy]acetate (226 mg, 605 μmol, 1.2 equiv) in DMF (2 mL) was added K₂CO₃ (139 mg, 1.01 mmol, 2.0 equiv) and 5-[[3-ethyl-5-[(2S)-2-(2-hydroxyethyl)-1-piperidyl]pyrazolo[1,5-a]pyrimidin-7-yl]amino methyl]pyridin-2-ol (200 mg, 504 μmol, 1.0 equiv). The mixture was stirred at 50° C. for 12 h and concentrated. The residue was purified by prep-HPLC (column: Waters Xbridge 150*25 mm*5 μm; mobile phase: [water (NH₄HCO₃)-ACN]; B %: 53%-83%, 8 min) to give tert-butyl 2-[2-[2-[5-[3-ethyl-5-[(2S)-2-(2-hydroxyethyl)-1-piperidyl]pyrazolo[1,5-a]pyrimidin-7-yl]amino methyl]-2-pyridyl]oxy]ethoxy]ethoxy]acetate (68 mg, 22% yield).

Step 2. To a solution of tert-butyl 2-[2-[2-[5-[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]acetate (60 mg, 100 μmol, 1.0 equiv) in DCM (1.5 mL) was added TFA (0.5 mL), the mixture was stirred at 25° C. for 1 h and concentrated to give crude 2-[2-[2-[5-[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]acetic acid (54 mg, 99 μmol) which was used into the next step without further purification.

Step 3. To a solution of 2-[2-[2-[[5-[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]acetic acid (54 mg, 99 μmol, 1.0 equiv) and (2S)—N-[(1S)-1-(2-chlorophenyl)-2-[(3,3-difluorocyclobutyl)amino]-2-oxo-ethyl]-1-(4-cyano-2-pyridyl)-N-(3,5-difluorophenyl)-6-oxo-piperazine-2-carboxamide (61 mg, 99 μmol, 1.0 equiv) in DMF (1 mL) was added DIEA (64 mg, 497 μmol, 5.0 equiv) and HATU (56 mg, 149 μmol, 1.5 equiv), the mixture was stirred at 25° C. for 12 h and concentrated. The residue was purified by prep-HPLC (column: Unisil 3-100 C18 Ultra 150*50 mm*3 μm; mobile phase: [water (FA)-ACN]; B %: 37%-67%,7 min) to give (2S)—N-[(1S)-1-(2-chlorophenyl)-2-[(3,3-difluorocyclobutyl)amino]-2-oxo-ethyl]-1-(4-cyano-2-pyridyl)-N-(3,5-difluorophenyl)-4-[2-[2-[2-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]acetyl]-6-oxo-piperazine-2-carboxamide (66 mg, 57% yield). ¹H NMR (400 MHZ, DMSO-d₆): δ 9.05-8.76 (m, 1H), 8.73-8.63 (m, 1H), 8.33-8.15 (m, 2H), 8.02-7.83 (m, 1H), 7.78-7.66 (m, 3H), 7.50-7.42 (m, 1H), 7.31-7.08 (m, 3H), 6.99-6.89 (m, 1H), 6.88-6.49 (m, 2H), 6.42-6.08 (m, 1H), 5.69-5.55 (m, 1H), 5.06-4.81 (m, 1H), 4.64-4.41 (m, 5H), 4.39-4.14 (m, 5H), 4.08-3.90 (m, 2H), 3.85-3.54 (m, 7H), 3.46-3.35 (m, 3H), 3.32-3.24 (m, 2H), 3.02-2.80 (m, 3H), 2.47-2.15 (m, 3H), 1.88-1.76 (m, 1H), 1.73-1.50 (m, 6H), 1.42-1.31 (m, 1H), 1.22-1.09 (m, 3H). LC-MS: MS (ES⁺): RT=2.311 min, m/z=1139.2 [M+H⁺]; LCMS method 25.

### Example 76—the Synthetic Route for I-98

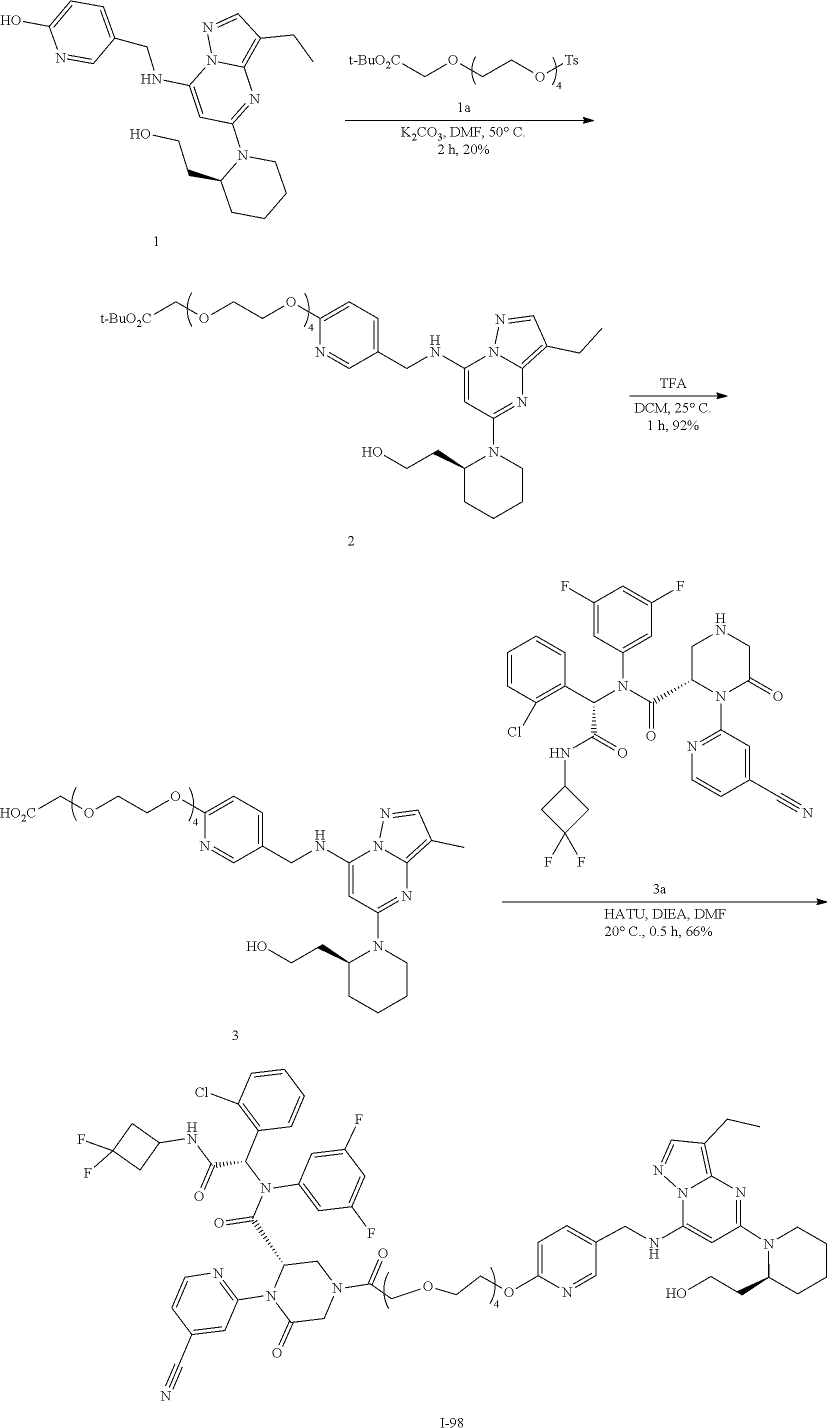

General Information: Synthetic route for compound 1a is described in KR102160377, 2020, B1. Synthetic route for compound 3a is described in WO2013/107291,2013, A¹.

Step 1. To a solution of tert-butyl 2-[2-[2-[2-[2-(p-tolylsulfonyloxy)ethoxy]ethoxy]ethoxy]ethoxy]acetate (200 mg, 432 μmol, 1.2 equiv) in DMF (1 mL) was added K₂CO₃ (100 mg, 720 μmol, 2.0 equiv) and 5-[[3-ethyl-5-[(2S)-2-(2-hydroxyethyl)-1-piperidyl]pyrazolo[1,5-a]pyrimidin-7-yl]amino]methyl]pyridin-2-ol (143 mg, 360 μmol, 1.0 equiv). The mixture was stirred at 50° C. for 2 h. The mixture was concentrated. The residue was purified by prep-HPLC (column: Unisil 3-100 C18 Ultra 150*50 mm*3 μm; mobile phase: [water (FA)-ACN]; B %: 28%-58%,7 min) to give tert-butyl 2-[2-[2-[2-[2-[5-[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]acetate (50 mg, 20% yield).

Step 2. A mixture of tert-butyl 2-[2-[2-[2-[2-[5-[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]acetate (40 mg, 58 μmol, 1.0 equiv) and TFA (100 μL) in DCM (1 mL) was degassed and purged with N₂ for 3 times, and then the mixture was stirred at 25° C. for 1 h under N₂ atmosphere. The mixture was filtered and the filtrate was concentrated. The product was used directly for next step.

Step 3. To a solution of 2-[2-[2-[2-[2-[5-[[3-ethyl-5-[(2S)-2-(2-hydroxyethyl)-1-piperidyl]pyrazolo[1,5-a]pyrimidin-7-yl]amino]methyl]-2 pyridyl]oxy]ethoxy]ethoxy]ethoxy]ethoxy]acetic acid (40 mg, 53.7 μmol, 1.0 equiv), (2S)—N-[(1S)-1-(2-chlorophenyl)-2-[(3,3-difluorocyclobutyl)amino]-2-oxo-ethyl]-1-(4-cyano-2-pyridyl)-N-(3,5-difluorophenyl)-6-oxo-piperazine-2-carboxamide (40 mg, 54 μmol, 1.0 equiv, TFA salt) in DMF (1 mL) was added HATU (31 mg, 80 μmol, 1.5 equiv) and DIEA (21 mg, 161 μmol, 3.0 equiv). The mixture was stirred at 20° C. for 0.5 h. The mixture was concentrated and purified by prep-HPLC (column:

Unisil 3-100 C18 Ultra 150*50 mm*3 μm; mobile phase: [water (FA)-ACN]; B %: 37%-67%,7 min) to give (2S)—N-[1-(2-chlorophenyl)-2-[(3,3-difluorocyclobutyl)amino]-2-oxo-ethyl]-1-(4-cyano-2-pyridyl)-N-(3,5-difluorophenyl)-4-[2-[2-[2-[2-[2-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]acetyl]-6-oxo-piperazine-2-carboxamide (43.8 mg, 66% yield). ¹H NMR (400 MHz, MeOD) δ 8.73 (d, J=5.0 Hz, 1H), 8.70-8.62 (m, 1H), 8.38-8.28 (m, 1H), 8.19 (s, 1H), 8.02-7.90 (m, 1H), 8.03-7.64 (m, 2H), 7.58-7.47 (m, 1H), 7.46-7.37 (m, 1H), 7.30-7.18 (m, 1H), 7.14-6.71 (m, 5H), 6.55-6.26 (m, 1H), 5.58 (s, 1H), 5.14 (s, 1H), 4.98 (s, 1H), 4.71 (s, 3H), 4.60-4.53 (m, 2H), 4.44-4.33 (m, 3H), 4.30 (s, 1H), 4.27-4.16 (m, 1H), 4.04-3.90 (m, 2H), 3.85-3.75 (m, 2H), 3.75-3.72 (m, 1H), 3.70 (s, 9H), 3.60-3.51 (m, 4H), 3.47-3.37 (m, 1H), 3.18-3.04 (m, 1H), 2.97-2.72 (m, 2H), 2.62-2.51 (m, 2H), 2.50-2.28 (m, 2H), 2.19-2.06 (m, 1H), 1.83-1.65 (m, 6H), 1.25-1.20 (m, 3H). LC-MS: MS (ES⁺): RT=2.497 min, m/z=1224.3 [M+H⁺]; LCMS method 25.

### Example 77—the Synthetic Route for I-99

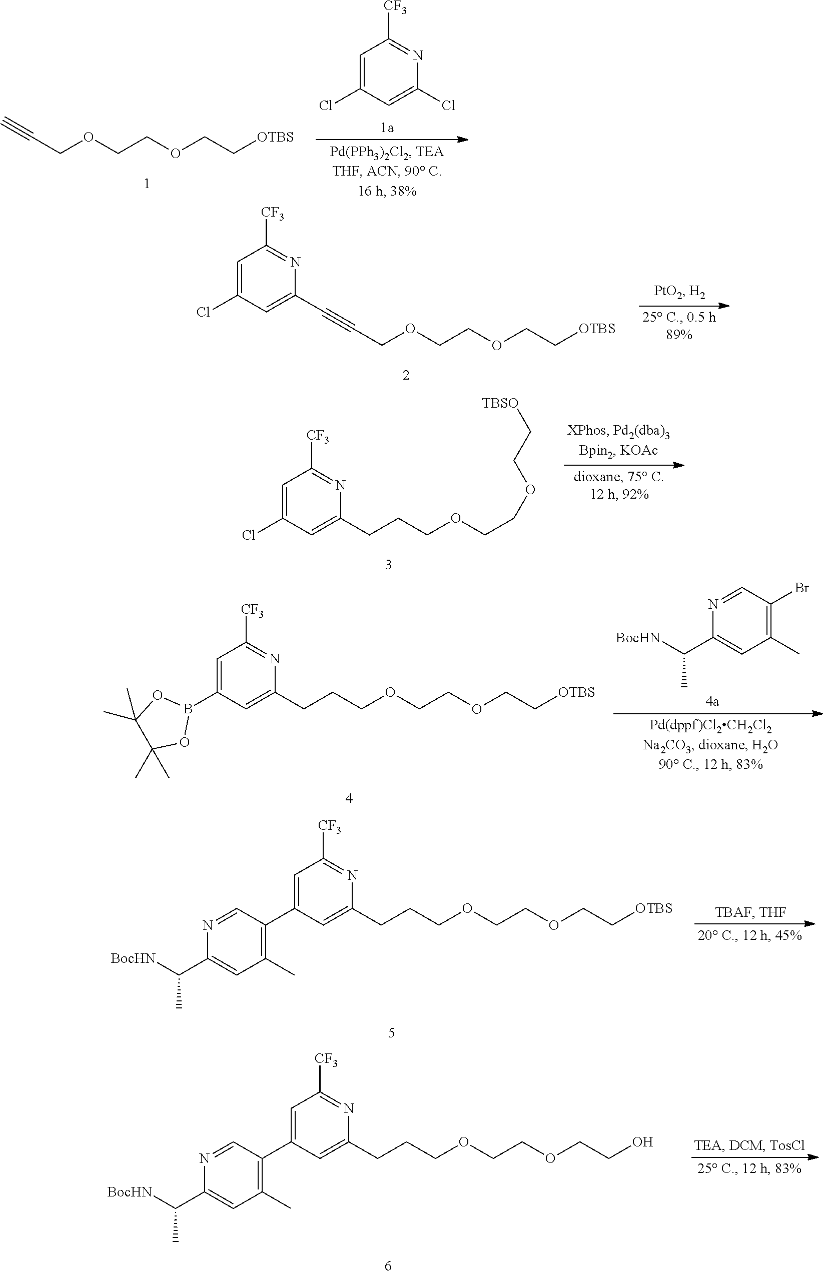

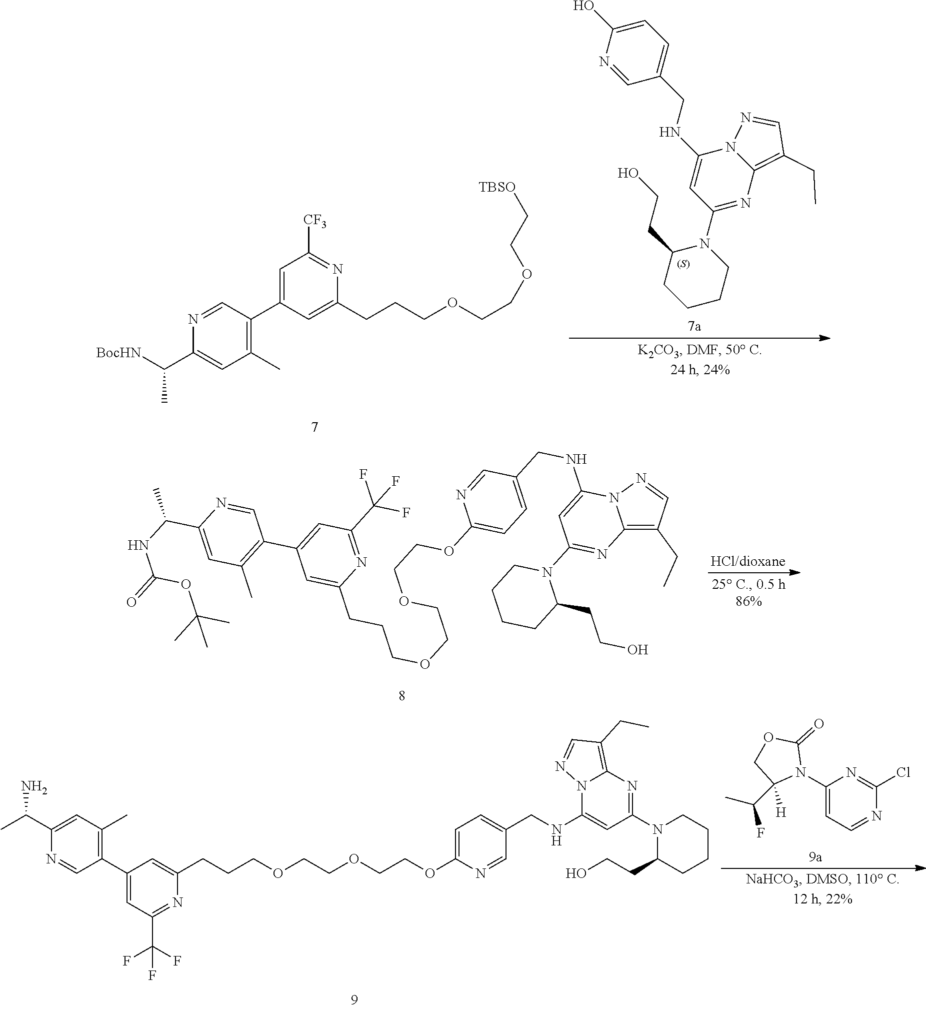

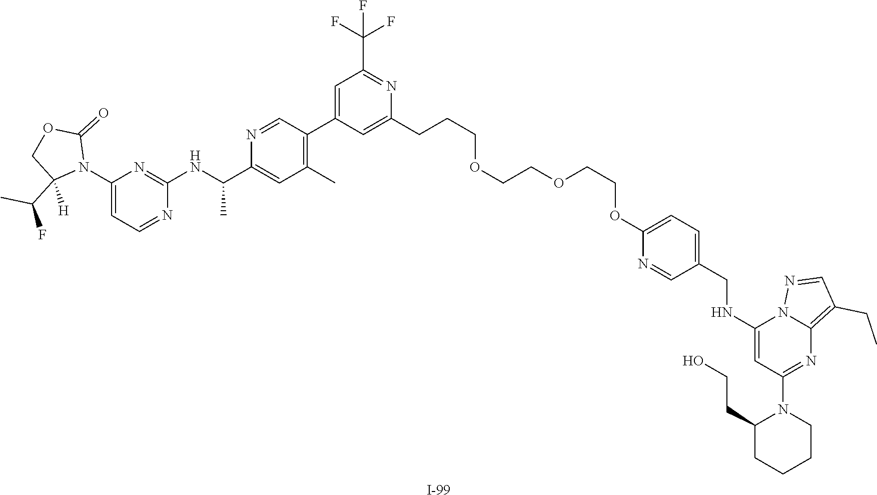

General Information: Synthetic route for compound 4a is described in WO2014/141104, 2014, A1.

Step 1. A mixture of 2,4-dichloro-6-(trifluoromethyl) pyridine (193 mg, 893 μmol, 1.0 equiv), tert-butyl-dimethyl-[2-(2-prop-2-ynoxyethoxy)ethoxy]silane (300 mg, 1.2 mmol, 1.3 equiv), Pd(PPh₃)2Cl₂ (63 mg, 89 μmol, 0.1 equiv), TEA (271 mg, 2.6 mmol, 3.0 equiv) and CuI (17 mg, 89 μmol, 0.1 equiv) in THF (4 mL) and MeCN(8 mL) was degassed and purged with N₂ for 3 times, and then the mixture was stirred at 90° C. for 16 h under N₂ atmosphere. The mixture was concentrated and purified by prep-HPLC (column: Waters Xbridge BEH C18 150*25 mm*5 μm; mobile phase: [water (NH₄HCO₃)-ACN]; B %: 70%-100%,12 min) to give tert-butyl-[2-[2-[3-[4-chloro-6-(trifluoromethyl)-2-pyridyl]prop-2-ynoxy]ethoxy]ethoxy]-dimethyl-silane (150 mg, 38% yield).

Step 2. To a solution of tert-butyl-[2-[2-[3-[4-chloro-6-(trifluoromethyl)-2-pyridyl]prop-2-ynoxy]ethoxy]ethoxy]-dimethyl-silane (100 mg, 228 μmol, 1.0 equiv), PtO2 (30 mg, 132 μmol, 5.79e-1 equiv) and H2 (460.3 μg, 228.3 μmol, 1.0 equiv) in THF (2 mL). The mixture was stirred under H₂ (15 psi) at 25° C. for 0.5 h and concentrated. The residue was purified by prep-TLC (SiO2, PE: EA=5:1) to give tert-butyl-[2-[2-[3-[4-chloro-6-(trifluoromethyl)-2-pyridyl]propoxy]ethoxy]ethoxy]-dimethyl-silane (90 mg, 89% yield).

Step 3. A mixture of tert-butyl-[2-[2-[3-[4-chloro-6-(trifluoromethyl)-2-pyridyl]propoxy]ethoxy]ethoxy]-dimethyl-silane (90 mg, 204 μmol, 1.0 equiv),4,4,5,5-tetramethyl-2-(4,4,5,5-tetramethyl-1,3,2-dioxaborolan-2-yl)-1,3,2-dioxaborolane (78 mg, 305 μmol, 1.5 equiv) and KOAc (60 mg, 611 μmol, 3.0 equiv) and Pd₂ (dba)3 (9.3 mg, 10 μmol, 0.05 equiv) XPhos (19 mg, 41 μmol, 0.2 equiv) in dioxane (2 mL) was degassed and purged with N₂ for 3 times, and then the mixture was stirred at 75° C. for 12 h under N₂ atmosphere. The mixture was filtered and concentrated. The residue was purified by prep-TLC (SiO2, PE: EA=5:1) to give tert-butyl-dimethyl-[2-[2-[3-[4-(4,4,5,5-tetramethyl-1,3,2-dioxaborolan-2-yl)-6-(trifluoromethyl)-2-pyridyl]propoxy]ethoxy]ethoxy]silane (100 mg, 92% yield).

Step 4. To a solution of tert-butyl-dimethyl-[2-[2-[3-[4-(4,4,5,5-tetramethyl-1,3,2-dioxaborolan-2-yl)-6-(trifluoromethyl)-2-pyridyl]propoxy]ethoxy]ethoxy]silane (350 mg, 656 μmol, 1.0 equiv), tert-butyl N-[(1S)-1-(5-bromo-4-methyl-2-pyridyl) ethyl]carbamate (207 mg, 656 μmol, 1.0 equiv), Pd(dppf) C12.CH₂Cl₂ (53.7 mg, 66 μmol, 0.1 equiv) and Na₂CO₃ (208 mg, 1.9 mmol, 3.0 equiv) in dioxane (2 mL) and H₂O (0.2 mL) was degassed and purged with N₂ for 3 times. The mixture was stirred at 90° C. for 12 h. The reaction mixture was filtered and the filtrate was concentrated. The residue was purified by prep-TLC (SiO2, PE: EA=5:1) to give tert-butyl N-[(1S)-1-[5-[2-[3-[2-[2-[tert-butyl(dimethyl)silyl]oxyethoxy]ethoxy]propyl]-6-(trifluoromethyl)-4-pyridyl]-4-methyl-2-pyridyl]ethyl]carbamate (350 mg, 83% yield).

Step 5. To a solution of tert-butyl N-[(1S)-1-[5-[2-[3-[2-[2-[tert-butyl(dimethyl)silyl]oxyethoxy]ethoxy]propyl]-6-(trifluoromethyl)-4-pyridyl]-4-methyl-2-pyridyl]ethyl]carbamate (350 mg, 545 μmol, 1.0 equiv) in THF (2 mL) was added TBAF (1 M, 1.6 mL, 3.0 equiv). The mixture was stirred at 20° C. for 12 h. The mixture was concentrated and purified by prep-TLC (SiO2, DCM: MeOH=10:1) to give tert-butyl N-[(1S)-1-[5-[2-[3-[2-(2-hydroxyethoxy)ethoxy]propyl]-6-(trifluoromethyl)-4-pyridyl]-4-methyl-2-pyridyl]ethyl]carbamate (130 mg, 45% yield).

Step 6. To a solution of tert-butyl N-[(1S)-1-[5-[2-[3-[2-(2-hydroxyethoxy)ethoxy]propyl]-6-(trifluoromethyl)-4-pyridyl]-4-methyl-2-pyridyl]ethyl]carbamate (130 mg, 246 μmol, 19.4 mL, 1.0 equiv) in DCM (2 mL) was added TosCl (141 mg, 739 μmol, 3.0 equiv) and TEA (125 mg, 1.23 mmol, 5 eq). The mixture was stirred at 25° C. for 12 h. The mixture was concentrated and purified by prep-TLC (SiO2, PE: EA=1:1) to give 2-[2-[3-[4-[6-[(1S)-1-(tert-butoxycarbonylamino)ethyl]-4-methyl-3-pyridyl]-6-(trifluoromethyl)-2-pyridyl]propoxy]ethoxy]ethyl 4-methylbenzenesulfonate (140 mg, 83% yield).

Step 7. To a solution of 2-[2-[3-[4-[6-[(1S)-1-(tert-butoxycarbonylamino)ethyl]-4-methyl-3-pyridyl]-6-(trifluoromethyl)-2-pyridyl]propoxy]ethoxy]ethyl 4-methylbenzenesulfonate (140 mg, 205.4 μmol, 1.0 equiv) in DMF (1 mL) was added K₂CO₃ (28.4 mg, 205 μmol, 1.0 equiv) and 5-[[3-ethyl-5-[(2S)-2-(2-hydroxyethyl)-1-piperidyl]pyrazolo[1,5-a]pyrimidin-7-yl]amino]methyl]pyridin-2-ol (81 mg, 205 μmol, 1.0 equiv). The mixture was stirred at 50° C. for 2 h. The mixture was filtered and the filtrate was concentrated. The residue was purified by prep-HPLC (column: Unisil 3-100 C18 Ultra 150*50 mm*3 μm; mobile phase: [water (FA)-ACN]; B %: 39%-69%,7 min) to give tert-butyl N-[(1S)-1-[5-[2-[3-[2-[2-[[5-[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]propyl]-6-(trifluoromethyl)-4-pyridyl]-4-methyl-2-pyridyl]ethyl]carbamate (45 mg, 24% yield).

Step 8. A mixture of tert-butyl N-[(1S)-1-[5-[2-[3-[2-[2-[5-[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]propyl]-6-(trifluoromethyl)-4-pyridyl]-4-methyl-2-pyridyl]ethyl]carbamate (10 mg, 11 μmol, 1.0 equiv) and HCl/dioxane (4 M, 2.7 μL, 1.0 equiv) in DCM (1 mL) was degassed and purged with N₂ for 3 times, and then the mixture was stirred at 25° C. for 1 h under N₂ atmosphere. The mixture was filtered and the filtrate was concentrated. The product was used directly for next step.

Step 9. To a solution of 2-[(2S)-1-[7-[[6-[2-[2-[3-[4-[6-[(1S)-1-aminoethyl]-4-methyl-3-pyridyl]-6-(trifluoromethyl)-2-pyridyl]propoxy]ethoxy]ethoxy]-3-pyridyl]methylamino]-3-ethyl-pyrazolo[1,5-a]pyrimidin-5-yl]-2-piperidyl]ethanol (30 mg, 36 μmol, 1.0 equiv, HCl) in DMSO (1 mL) was added NaHCO₃ (15 mg, 178 μmol, 5.0 equiv) and (4R)-3-(2-chloropyrimidin-4-yl)-4-[(1S)-1-fluoroethyl]oxazolidin-2-one (18 mg, 71 μmol, 2.0 equiv) The mixture was stirred at 110° C. for 12 h. The reaction mixture was filtered and the filtrate was concentrated. The residue was purified by prep-HPLC (column: Unisil 3-100 C18 Ultra 150*50 mm*3 μm; mobile phase: [water (FA)-ACN]; B %: 36%-66%,7 min) to give (4R)-3-[2-[(1S)-1-[5-[2-[3-[2-[2-[5-[[3-ethyl-5-[(2S)-2-(2-hydroxyethyl)-1-piperidyl]pyrazolo[1,5-a]pyrimidin-7-yl]amino methyl]-2-pyridyl]oxy]ethoxy]ethoxy]propyl]-6-(trifluoromethyl)-4-pyridyl]-4-methyl-2-pyridyl]ethyl]amino]pyrimidin-4-yl]-4-[(1S)-1-fluoroethyl]oxazolidin-2-one (7.98 mg, 22% yield). ¹H NMR (400 MHZ, MeOD) δ 8.36 (s, 1H), 8.21-8.10 (m, 2H), 7.69 (S, 1H), 7.63 (s, 1H), 7.59 (s, 1H), 7.52 (s, 1H), 7.42-7.36 (m, 2H), 6.72 (d, J=8.4 Hz, 1H), 5.51 (s, 1H), 5.13-5.01 (m, 1H), 4.83-4.73 (m, 2H), 4.72-4.65 (m, 1H), 4.59 (s, 1H), 4.54-4.46 (m, 3H), 4.45-4.38 (m, 1H), 4.36-4.31 (m, 2H), 4.01 (s, 1H), 3.80-3.75 (m, 2H), 3.67-3.60 (m, 2H), 3.56 (s, 2H), 3.54-3.47 (m, 3H), 3.38 (s, 1H), 3.04-2.91 (m, 3H), 2.60-2.48 (m, 2H), 2.25 (s, 3H), 2.15-1.96 (m, 3H), 1.77-1.62 (m, 6H), 1.56 (s, 3H), 1.53-1.44 (m, 1H), 1.22 (s, 3H), 1.18-1.03 (m, 2H). LC-MS: MS (ES⁺): RT=2.220 min, m/z=1015.5 [M+H⁺]; LCMS method 25.

### Example 78—the Synthetic Route for I-100

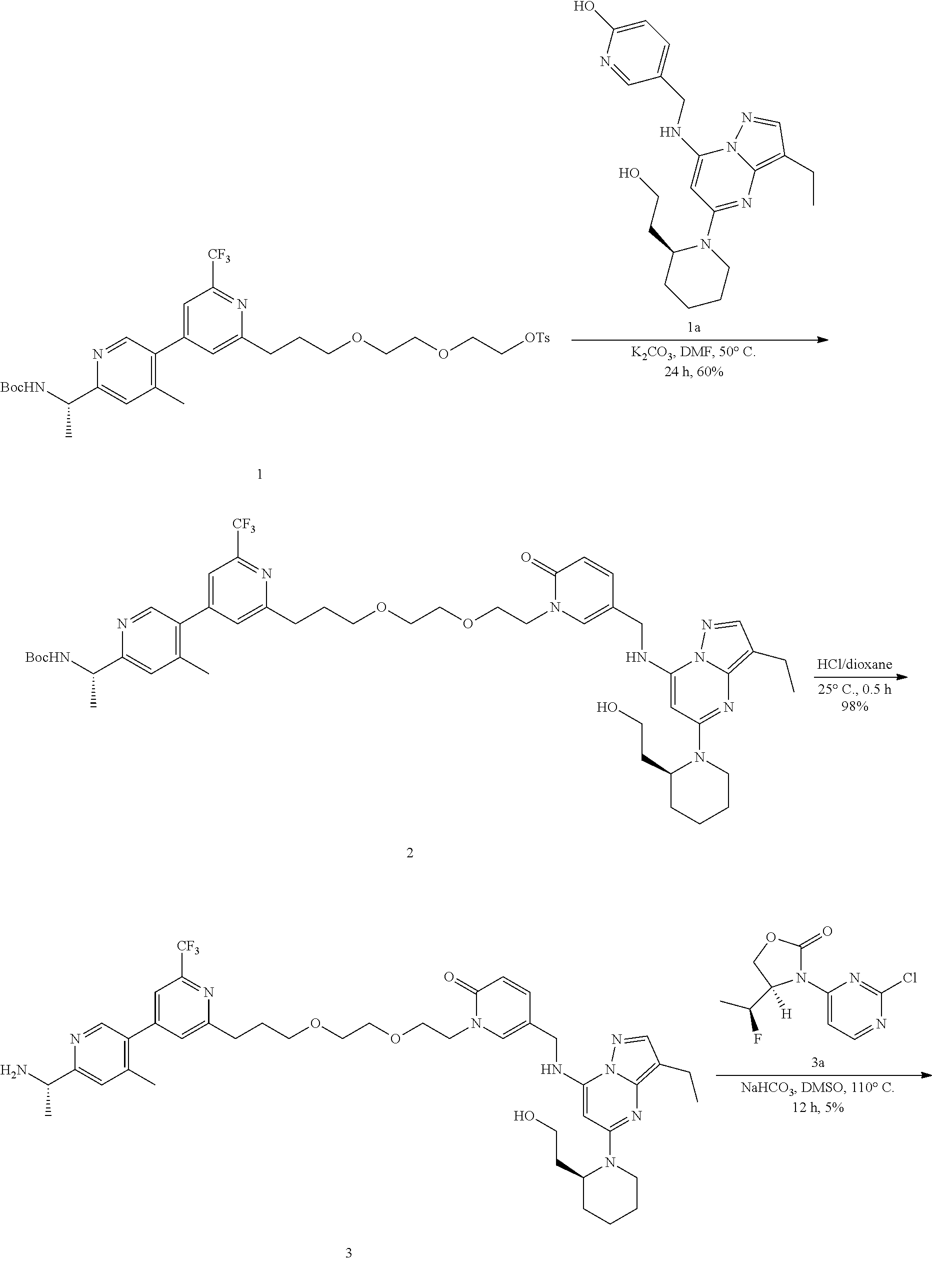

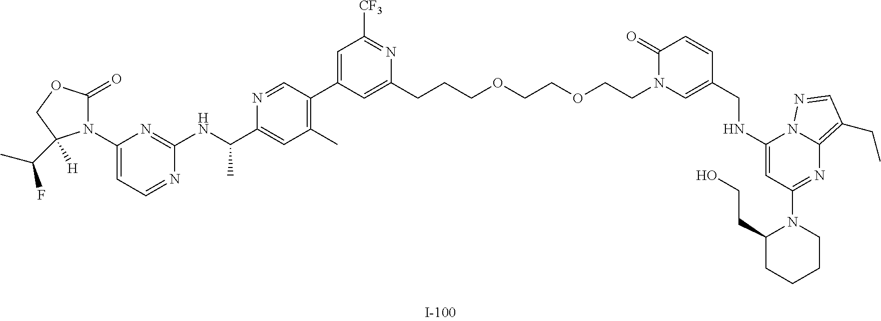 [SMILES: CCc1cnn2c(NCc3ccc(=O)n(CCOCCOCCCc4cc(-c5cnc([C@H](C)Nc6nccc(N7C(=O)OC[C@@H]7[C@H](C)F)n6)cc5C)cc(C(F)(F)F)n4)c3)cc(N3CCCC[C@H]3CCO)nc12]

Step 1. To a solution of 2-[2-[3-[4-[6-[(1S)-1-(tert-butoxycarbonylamino)ethyl]-4-methyl-3-pyridyl]-6-(trifluoromethyl)-2-pyridyl]propoxy]ethoxy]ethyl 4-methyl benzenesulfonate (140 mg, 205 μmol, 1.0 equiv) in DMF (1 mL) was added K₂CO₃ (28.4 mg, 205 μmol, 1.0 equiv) and 5-[[[3-ethyl-5-[(2S)-2-(2-hydroxyethyl)-1-piperidyl]pyrazolo[1,5-a]pyrimidin-7-yl]amino methyl]pyridin-2-ol (81 mg, 205 μmol, 1.0 equiv). The mixture was stirred at 50° C. for 2 h. The mixture was filtered and the filtrate was concentrated. The residue was purified by prep-HPLC (column: Unisil 3-100 C18 Ultra 150*50 mm*3 μm; mobile phase: [water (FA)-ACN]; B %: 39%-69%,7 min) to give tert-butyl N-[(1S)-1-[5-[2-[3-[2-[2-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]propyl]-6-(trifluoromethyl)-4-pyridyl]-4-methyl-2-pyridyl]ethyl]carbamate (45 mg, 24% yield).

Step 2. A mixture of tert-butyl N-[(1S)-1-[5-[2-[3-[2-[2-[5-[[[3-ethyl-5-[(2S)-2-(2-hydroxyethyl)-1-piperidyl]pyrazolo[1,5-a]pyrimidin-7-yl]amino]methyl]-2-oxo-1-pyridyl]ethoxy]ethoxy]propyl]-6-(trifluoromethyl)-4-pyridyl]-4-methyl-2-pyridyl]ethyl]carbamate (50 mg, 55 μmol, 1.0 equiv) and HCl/dioxane (4 M, 14 μL, 1.0 equiv) in DCM (1 mL) was degassed and purged with N₂ for 3 times, and then the mixture was stirred at 25° C. for 1 h under N₂ atmosphere. The mixture was filtered and the filtrate was concentrated. The product was used directly for next step.

Step 3. To a solution of 1-[2-[2-[3-[4-[6-[(1S)-1-aminoethyl]-4-methyl-3-pyridyl]-6-(trifluoromethyl)-2-pyridyl]propoxy]ethoxy]ethyl]-5-[[[3-ethyl-5-[(2S)-2-(2-hydroxyethyl)-1-piperidyl]pyrazolo[1,5-a]pyrimidin-7-yl]amino]methyl]pyridin-2-one (46 mg, 55 μmol, 1.0 equiv, HCl) in DMSO (0.5 mL) was added NaHCO₃ (23 mg, 273 μmol, 5.0 equiv) and (4R)-3-(2-chloropyrimidin-4-yl)-4-[(1S)-1-fluoroethyl]oxazolidin-2-one (27 mg, 109 μmol, 2.0 equiv). The mixture was stirred at 110° C. for 12 h. The mixture was filtered and the filtrate was concentrated. The residue was purified by prep-HPLC (column: Waters Xbridge BEH C18 150*25 mm*5 μm; mobile phase: [water (NH₄HCO₃)-ACN]; B %: 56%-86%,10 min) to give (4R)-3-[2-[[(1S)-1-[5-[2-[3-[2-[2-[5-[[3-ethyl-5-[(2S)-2-(2-hydroxyethyl)-1-piperidyl]pyrazolo[1,5-a]pyrimidin-7-yl]amino methyl]-2-oxo-1-pyridyl]ethoxy]ethoxy]propyl]-6-(trifluoromethyl)-4-pyridyl]-4-methyl-2-pyridyl]ethyl]amino]pyrimidin-4-yl]-4-[(1S)-1-fluoroethyl]oxazolidin-2-one (3.05 mg, 5% yield). ¹H NMR (400 MHZ, MeOD) δ 8.38 (s, 1H), 8.21-8.13 (m, 1H), 7.71 (s, 1H), 7.63-7.55 (m, 3H), 7.46 (s, 1H), 7.40 (s, 2H), 6.53 (d, J=8.8 Hz, 1H), 5.53 (s, 1H), 5.14-4.98 (m, 2H), 4.80-4.73 (m, 3H), 4.67-4.55 (m, 3H), 4.53-4.47 (m, 1H), 4.43 (s, 1H), 4.38 (s, 2H), 4.15-4.03 (m, 3H), 3.76-3.69 (m, 2H), 3.57-3.51 (m, 1H), 3.48 (s, 2H), 3.39 (s, 1H), 3.38-3.33 (m, 4H), 2.98 (s, 1H), 2.89 (t, J=7.6 Hz, 2H), 2.56-2.48 (m, 2H), 2.28 (s, 3H), 2.14-2.01 (m, 1H), 1.98-1.88 (m, 2H), 1.75-1.61 (m, 6H), 1.58 (s, 3H), 1.19 (s. 4H). LC-MS: MS (ES⁺): RT=2.489 min, m/z=1015.5] M+H⁺]; LCMS method 25.

### Example 79—the Synthetic Route for I-101

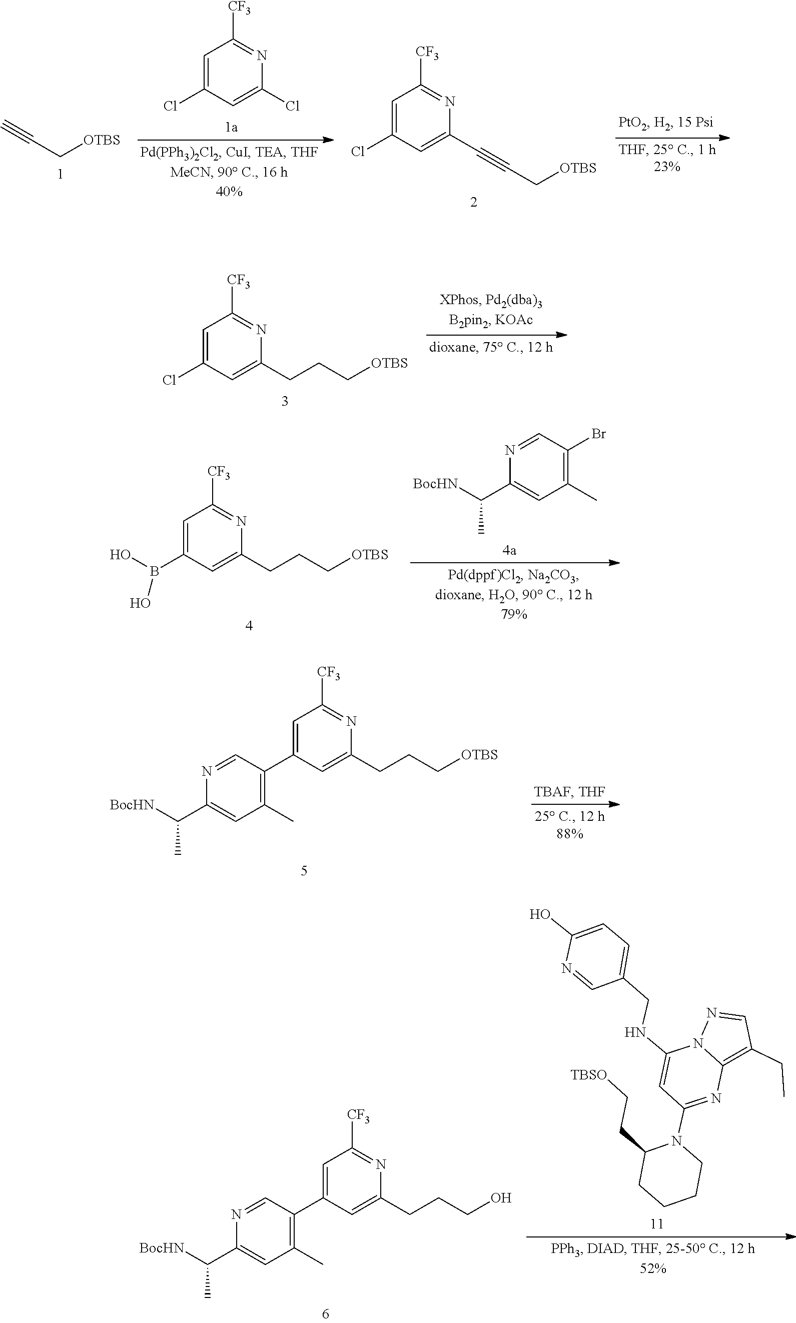

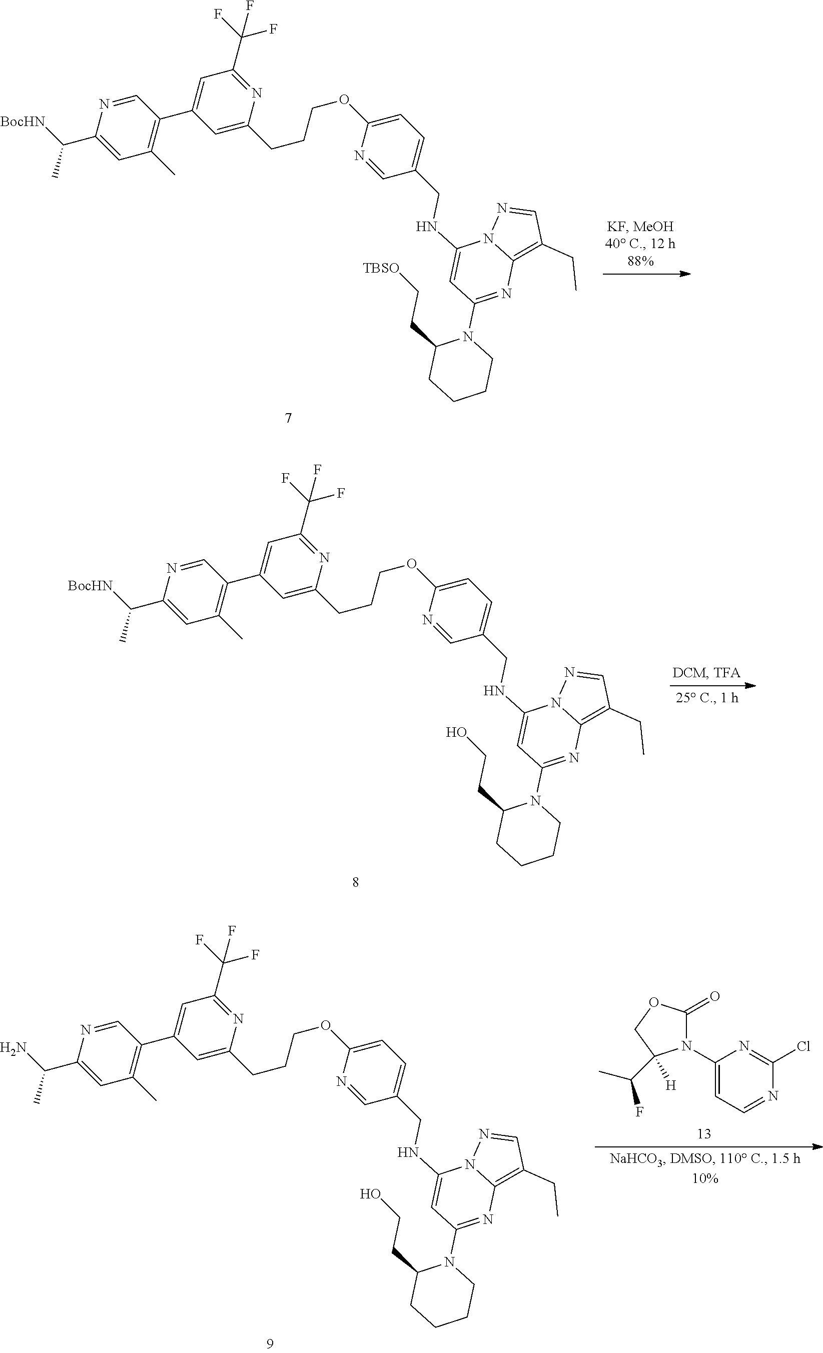

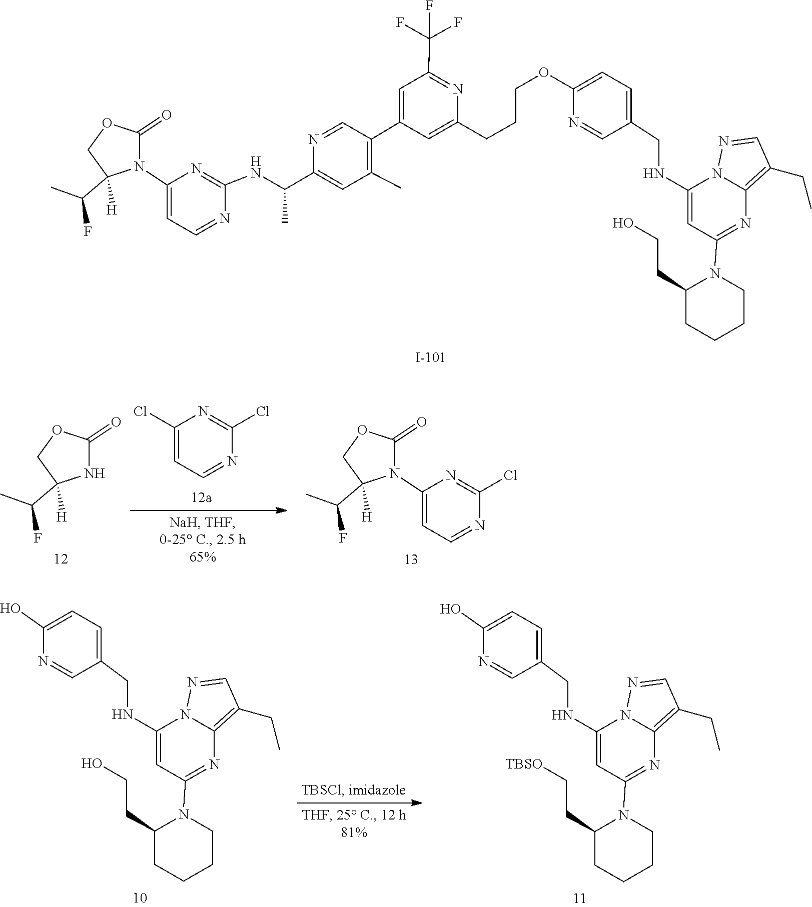

General Information: Compound 12 is described in ACS Medicinal Chemistry Letters, 2017, vol. 8, #10, p. 1116-1121.

Step 1. A mixture of 2,4-dichloro-6-(trifluoromethyl) pyridine (5 g, 23.15 mmol, 1 eq), tert-butyl-dimethyl-prop-2-ynoxy-silane (9.07 g, 53.2 mmol, 10.8 mL, 2.3 equiv), Pd(PPh₃)2Cl₂ (1.62 g, 2.31 mmol, 0.1 equiv), CuI (441 mg, 2.31 mmol, 0.1 equiv) and TEA (7.03 g, 69.5 mmol, 3.0 equiv) in THF (40 mL) and MeCN(80 mL) was degassed and purged with N₂ for 3 times, and then the mixture was stirred at 90° C. for 16 h under N₂ atmosphere. The mixture was filtered and concentrated. The residue was purified by prep-HPLC (column: Phenomenex luna C18 (250*70 mm*10 μm); mobile phase: [water (FA)-ACN]; B %: 65%-86%, 35 min) to give tert-butyl-[3-[4-chloro-6-(trifluoromethyl)-2-pyridyl]prop-2-ynoxy]-dimethyl-silane (3.2 g, 40% yield) as a yellow liquid. ¹H NMR (400 MHZ, CDCl₃): δ 7.62 (d, J=1.6 Hz, 1H), 7.59 (d, J=1.6 Hz, 1H), 4.57 (s, 2H), 0.94 (s, 9H), 0.17 (s, 6H).

Step 2. To a solution of tert-butyl-[3-[4-chloro-6-(trifluoromethyl)-2-pyridyl]prop-2-ynoxy]-dimethyl-silane (3 g, 8.58 mmol, 1.0 equiv) in THF (30 mL) was added PtO2 (500 mg, 2.20 mmol, 4.4 equiv) under N₂. The mixture was stirred under H₂ (15 psi) at 25° C. for 1 h. The mixture was filtered and concentrated. The residue was purified by prep-HPLC (column: Waters Xbridge C18 150*50 mm*10 μm; mobile phase: [water (NH₄HCO₃)-ACN]; B %: 80%-100%, 10 min) to give tert-butyl-[3-[4-chloro-6-(trifluoromethyl)-2-pyridyl]propoxy]-dimethyl-silane (700 mg, 23% yield). ¹H NMR (400 MHZ, CDCl₃): δ 7.51 (d, J=1.6 Hz, 1H), 7.38 (d, J=1.2 Hz, 1H), 3.67 (t, J=6.0 Hz, 2H), 3.00-2.90 (m, 2H), 2.03-1.91 (m, 2H), 0.90 (s, 9H), 0.05 (s, 6H).

Step 3. To a solution of tert-butyl-[3-[4-chloro-6-(trifluoromethyl)-2-pyridyl]propoxy]-dimethyl-silane (700 mg, 1.98 mmol, 1.0 equiv) in dioxane (20 mL) was added BPD (753 mg, 2.97 mmol, 1.5 equiv), Pd₂ (dba)3 (181 mg, 198 μmol, 0.1 equiv), XPhos (189 mg, 396 μmol, 0.2 equiv) and KOAc (582 mg, 5.93 mmol, 3.0 equiv). The mixture was stirred at 75° C. for 12 h. The mixture was filtered and concentrated. The residue was purified by prep-TLC (Petroleum ether: Ethyl acetate=3/1) to give [2-[3-[tert-butyl(dimethyl)silyl]oxypropyl]-6-(trifluoromethyl)-4-pyridyl]boronic acid (500 mg, crude).

Step 4. To a solution of tert-butyl N-[(1S)-1-(5-bromo-4-methyl-2-pyridyl) ethyl]carbamate (434 mg, 1.38 mmol, 1.0 equiv), [2-[3-[tertbutyl(dimethyl)silyl]oxypropyl]-6-(trifluoromethyl)-4-pyridyl]boronic acid (500 mg, 1.38 mmol, 1.0 equiv), Pd(dppf) Cl₂.CH₂Cl₂ (112 mg, 138 μmol, 0.1 equiv) and Na₂CO₃ (438 mg, 4.13 mmol, 3.0 equiv) in dioxane (10 mL) and H₂O (1 mL) was degassed and purged with N₂ for 3 times. The mixture was stirred at 90° C. for 12 h. The mixture was filtered and concentrated. The residue was purified by prep-TLC (Petroleum ether: Ethyl acetate=3/1) to give tert-butyl N-[(1S)-1-[5-[2-[3-[tert-butyl(dimethyl) silyl]oxypropyl]-6-(trifluoromethyl)-4-pyridyl]-4-methyl-2-pyridyl]ethyl]carbamate (600 mg, 79% yield). ¹H NMR (400 MHZ, CDCl₃): δ 8.35 (s, 1H), 7.46 (d, J=1.2 Hz, 1H), 7.31 (s, 1H), 7.20 (s, 1H), 5.65-5.54 (m, 1H), 4.97-4.80 (m, 1H), 3.72 (t, J=6.4 Hz, 2H), 3.09-2.95 (m, 2H), 2.30 (s, 3H), 2.05-2.00 (m, 2H), 1.51-1.44 (m, 12H), 0.89 (s, 9H), 0.05 (s, 6H).

Step 5. To a solution of tert-butyl N-[(1S)-1-[5-[2-[3-[tert-butyl(dimethyl)silyl]oxypropyl]-6-(trifluoromethyl)-4-pyridyl]-4-methyl-2-pyridyl]ethyl]carbamate (600 mg, 1.08 mmol, 1.0 equiv) in THF (10 mL) was added TBAF (1 M, 3.3 mL, 3.0 equiv). The mixture was stirred at 25° C. for 12 h. The reaction mixture was diluted with water (30 mL) and extracted with ethyl acetate (30 mL*3). The combined organic layers were washed with brine (50 mL), dried over sodium sulfate, filtered and concentrated. The residue was purified by prep-TLC (Petroleum ether: Ethyl acetate=1/1) to give tert-butyl N-[(1S)-1-[5-[2-(3-hydroxypropyl)-6-(trifluoromethyl)-4-pyridyl]-4-methyl-2-pyridyl]ethyl]carbamate (420 mg, 88% yield).

Step 6. To a solution of tert-butyl N-[(1S)-1-[5-[2-(3-hydroxypropyl)-6-(trifluoromethyl)-4-pyridyl]-4-methyl-2-pyridyl]ethyl]carbamate (180 mg, 410 μmol, 1.0 equiv) in THF (5 mL) was added PPh₃ (0.5 M, 3.3 mL, 4.0 equiv), 5-[[5-[(2S)-2-[2-[tertbutyl(dimethyl)silyl]oxyethyl]-1-piperidyl]-3-ethyl-pyrazolo[1,5-a]pyrimidin-7-ylamino]methyl]pyridin-2-ol (209 mg, 410 μmol, 1.0 equiv) and DIAD (331 mg, 1.64 mmol, 4.0 equiv) at 25° C. The mixture was stirred at 50° C. for 12 h. The reaction mixture was diluted with water (15 mL) and extracted with ethyl acetate (20 mL*3). The combined organic layers were washed with brine (20 mL*3), dried over sodium sulfate, filtered and concentrated. The residue was purified by prep-TLC (Petroleum ether: Ethyl acetate=1/1) and prep-HPLC (column: Phenomenex Synergi Polar-RP 100*25 mm*4 μm; mobile phase: [water (TFA)-ACN]; B %: 46%-76%, 9 min) to give tert-butyl N-[(1S)-1-[5-[2-[3-[5-[ [5-[(2S)-2-[2-[tert-butyl(dimethyl)silyl]oxyethyl]-1-piperidyl]-3-ethyl-pyrazolo[1,5-a]pyrimidin-7-yl]amino methyl]-2-pyridyl]oxy]propyl]-6-(trifluoromethyl)-4-pyridyl]-4-methyl-2-pyridyl]ethyl]carbamate (200 mg, 52% yield). ¹H NMR (400 MHZ, CDCl₃): δ 8.29 (s, 1H), 8.13 (d, J=2.0 Hz, 1H), 7.64 (s, 1H), 7.60 (m, J=2.4, 8.8 Hz, 1H), 7.47 (s, 1H), 7.33 (s, 1H), 7.19 (s, 1H), 6.71 (d, J=8.8 Hz, 1H), 6.28-6.18 (m, 1H), 5.69 (d, J=7.2 Hz, 1H), 5.43 (s, 1H), 4.93-4.81 (m, 1H), 4.63 (d, J=1.6 Hz, 1H), 4.48-4.32 (m, 5H), 3.74-3.59 (m, 2H), 3.12 (t, J=7.6 Hz, 2H), 2.99-2.82 (m, 1H), 2.64 (q, J=7.6 Hz, 2H), 2.36-2.24 (m, 5H), 1.97-1.78 (m, 3H), 1.75-1.61 (m, 8H), 1.50-1.42 (m, 12H), 1.28-1.23 (m, 3H), 0.87 (s, 9H), 0.02 (d, J=1.2 Hz, 6H).

Step 7. To a solution of tert-butyl N-[(1S)-1-[5-[2-[3-[[5-[[5-[(2S)-2-[2-[tert-butyl(dimethyl)silyl]oxyethyl]-1-piperidyl]-3-ethylpyrazolo[1,5-a]pyrimidin-7-yl]amino]methyl]-2-pyridyl]oxy]propyl]-6-(trifluoromethyl)-4-pyridyl]-4-methyl-2-pyridyl]ethyl]carbamate (200 mg, 215 μmol, 1.0 equiv) in MeOH (3 mL) was added KF (125 mg, 2.15 mmol, 10.0 equiv). The mixture was stirred at 40° C. for 12 h. The reaction mixture was diluted with water (15 mL) and extracted with ethyl acetate (20 mL*3). The combined organic layers were washed with brine (20 mL*3), dried over sodium sulfate, filtered and concentrated to give tert-butyl N-[(1S)-1-[5-[2-[3-[[5-[[[3-ethyl-5-[(2S)-2-(2-hydroxyethyl)-1-piperidyl]pyrazolo[1,5-a]pyrimidin-7-yl]amino]methyl]-2-pyridyl]oxy]propyl]-6-(trifluoromethyl)-4-pyridyl]-4-methyl-2-pyridyl]ethyl]carbamate (200 mg, crude) as a yellow oil.

Step 8. To a solution of tert-butyl N-[(1S)-1-[5-[2-[3-[[5-[[3-ethyl-5-[(2S)-2-(2-hydroxyethyl)-1-piperidyl]pyrazolo[1,5-a]pyrimidin-7-yl]amino]methyl]-2-pyridyl]oxy]propyl]-6-(trifluoromethyl)-4-pyridyl]-4-methyl-2-pyridyl]ethyl]carbamate (150 mg, 183 μmol, 1.0 equiv) in DCM (2 mL) was added TFA (0.5 mL). The mixture was stirred at 25° C. for 1 h and concentrated. Saturated sodium bicarbonate solution (10 mL) was added and extracted with DCM (20 mL*3). The combined organic layers were washed with brine (20 mL), dried over sodium sulfate, filtered and concentrated to give 2-[(2S)-1-[7-[[6-[3-[4-[6-[(1S)-1-aminoethyl]-4-methyl-3-pyridyl]-6-(trifluoromethyl)-2-pyridyl]propoxy]-3-pyridyl]methylamino]-3-ethyl-pyrazolo[1,5-a]pyrimidin-5-yl]-2-piperidyl]ethanol (150 mg, crude).

Step 9. To a solution of 5-[[[3-ethyl-5-[(2S)-2-(2-hydroxyethyl)-1-piperidyl]pyrazolo[1,5-a]pyrimidin-7-yl]amino]methyl]pyridin-2-ol (500 mg, 1.26 mmol, 1.0 equiv) in THF (10 mL) was added TBSCI (228 mg, 1.51 mmol, 1.2 equiv) and imidazole (172 mg, 2.52 mmol, 2.0 equiv). The mixture was stirred at 25° C. for 12 h. The reaction mixture was diluted with water (20 mL) and extracted with DCM (20 mL*3). The combined organic layers were washed with brine (20 mL), dried over sodium sulfate, filtered and concentrated. The residue was purified by prep-TLC (Dichloromethane: Methanol=10/1) to give 5-[[5-[(2S)-2-[2-[tert-butyl(dimethyl)silyl]oxyethyl]-1-piperidyl]-3-ethyl-pyrazolo[1,5-a]pyrimidin-7-yl]amino methyl]pyridin-2-ol (520 mg, 81% yield). ¹H NMR (400 MHZ, CDCl₃): δ 13.41-12.63 (m, 1H), 7.64 (s, 1H), 7.50 (m, J=2.4, 9.6 Hz, 1H), 7.37 (d, J=2.0 Hz, 1H), 6.60 (d, J=9.6 Hz, 1H), 6.29 (t, J=5.2 Hz, 1H), 5.39 (s, 1H), 4.63 (s, 1H), 4.41 (d, J=12.0 Hz, 1H), 4.26 (d, J=5.6 Hz, 2H), 3.75-3.62 (m, 2H), 3.01-2.87 (m, 1H), 2.64 (q, J=7.6 Hz, 2H), 1.93-1.80 (m, 2H), 1.76-1.54 (m, 6H), 1.27 (t, J=7.6 Hz, 3H), 0.88 (s, 9H), 0.03 (d, J=1.6 Hz, 6H).

Step 10. To a cooled (0° C.) solution of (4R)-4-[(1S)-1-fluoroethyl]oxazolidin-2-one (2 g, 15 mmol, 1.0 equiv) and 2,4-dichloropyrimidine (2.69 g, 18.0 mmol, 1.2 equiv) in THF (50 mL) was added NaH (901 mg, 22.5 mmol, 60% purity, 1.5 equiv). The resulting mixture was stirred at 0° C. for 30 min and at 25° C. for 2 h. The reaction mixture was adjusted pH to 7 with AcOH at 0° C., diluted with water (50 mL) and extracted with ethyl acetate (50 mL*3). The combined organic layers were washed with brine (100 mL*3), dried over sodium sulfate, filtered and concentrated. The residue was purified by silica column chromatography on silica gel (Petroleum ether: Ethyl acetate from 50/1 to 2/1) to give (4R)-3-(2-chloropyrimidin-4-yl)-4-[(1S)-1-fluoroethyl]oxazolidin-2-one (2.4 g, 65% yield). ¹H NMR (400 MHZ, CDCl₃): δ 8.49 (d, J=6.0 Hz, 1H), 8.20 (d, J=6.0 Hz, 1H), 5.42-5.21 (m, 1H), 4.87-4.72 (m, 1H), 4.64 (m, J=3.6, 8.8 Hz, 1H), 4.54-4.44 (m, 1H), 1.50-1.34 (m, 3H).

Step 11. To a solution of 2-[(2S)-1-[7-[[6-[3-[4-[6-[(1S)-1-aminoethyl]-4-methyl-3-pyridyl]-6-(trifluoromethyl)-2-pyridyl]propoxy]-3-pyridyl]methylamino]-3-ethyl-pyrazolo[1,5-a]pyrimidin-5-yl]-2-piperidyl]ethanol (150 mg, 209 μmol, 1.0 equiv) in DMSO (1.5 mL) was added NaHCO₃ (88 mg, 1.04 mmol, 5.0 equiv) and (4R)-3-(2-chloropyrimidin-4-yl)-4-[(1S)-1-fluoroethyl]oxazolidin-2-one (77 mg, 313 μmol, 1.5 equiv). The mixture was stirred at 110° C. for 1.5 h. The mixture was concentrated and purified by prep-HPLC (column: Waters Xbridge C18 150*50 mm*10 μm; mobile phase: [water (NH₄HCO₃)-ACN]; B %: 62%-92%, 10 min) to give (4R)-3-[2-[[(1S)-1-[5-[2-[3-[[5-[[3-ethyl-5-[(2S)-2-(2-hydroxyethyl)-1-piperidyl]pyrazolo[1,5-a]pyrimidin-7-yl]amino]methyl]-2-pyridyl]oxy]propyl]-6-(trifluoromethyl)-4-pyridyl]-4-methyl-2-pyridyl]ethyl]amino]pyrimidin-4-yl]-4-[(1S)-1-fluoroethyl]oxazolidin-2-one (21 mg, 10% yield). ¹H NMR (400 MHZ, DMSO-d₆): δ 8.40 (s, 1H), 8.28-8.13 (m, 2H), 7.83 (t, J=6.4 Hz, 1H), 7.74 (m, J=2.4, 8.4 Hz, 2H), 7.70-7.61 (m, 2H), 7.32 (s, 1H), 7.24 (d, J=5.6 Hz, 1H), 6.73 (d, J=8.4 Hz, 1H), 5.58 (s, 1H), 5.07-4.51 (m, 4H), 4.45 (d, J=5.2 Hz, 4H), 4.31-4.19 (m, 3H), 3.40-3.34 (m, 1H), 3.30 (s, 3H), 3.01 (t, J=7.6 Hz, 2H), 2.86-2.75 (m, 1H), 2.47-2.43 (m, 2H), 2.23 (s, 3H), 2.19-2.14 (m, 2H), 1.82-1.75 (m, 1H), 1.69-1.56 (m, 4H), 1.55-1.46 (m, 5H), 1.41-1.26 (m, 2H), 1.16 (t, J=7.6 Hz, 3H), 1.12-1.00 (m, 2H). LC-MS: MS (ES⁺): RT=2.890 min, m/z=928.4 [M+H⁺]; LCMS method: 01.

### Example 80—the Synthetic Route for I-102

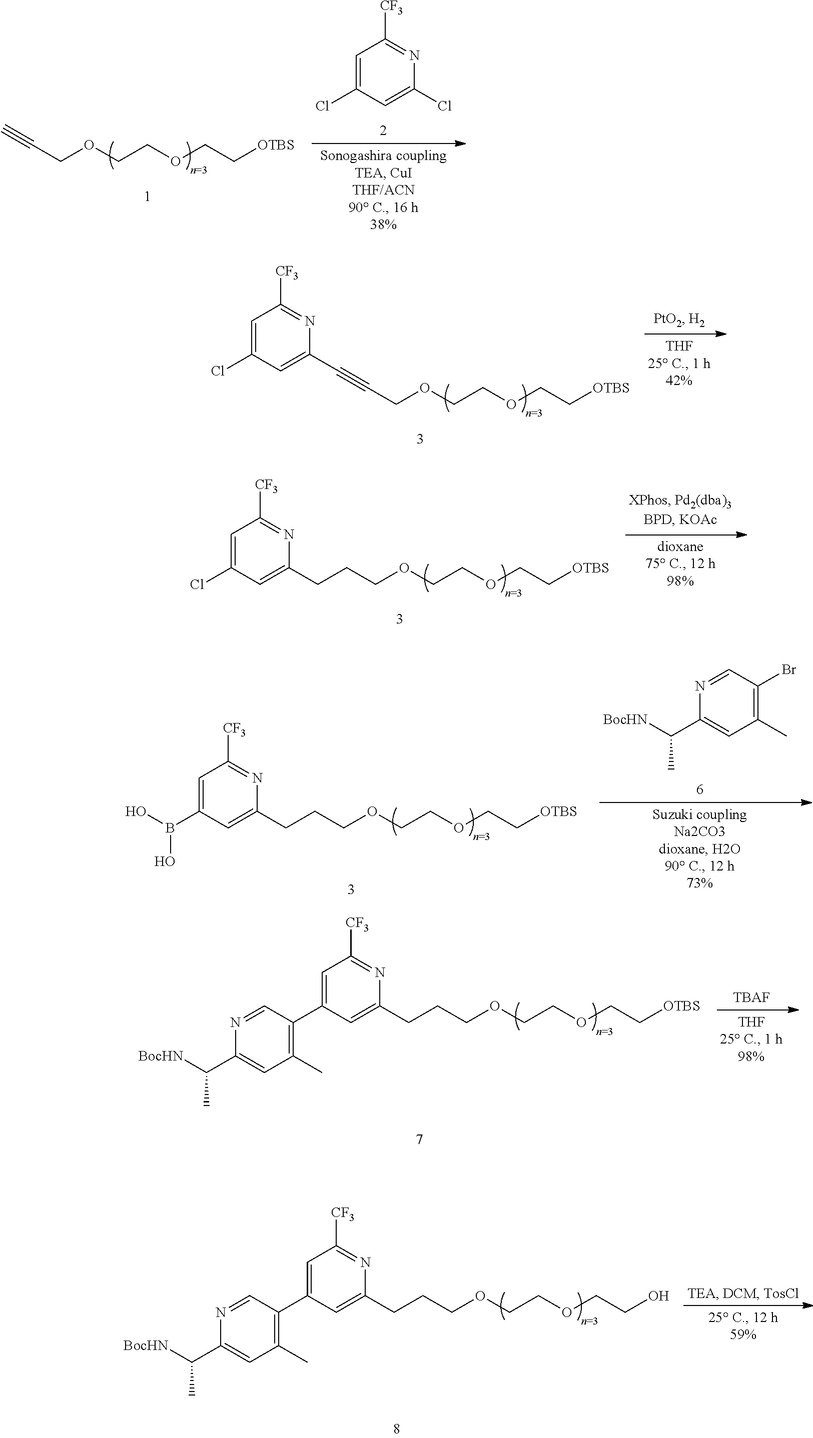

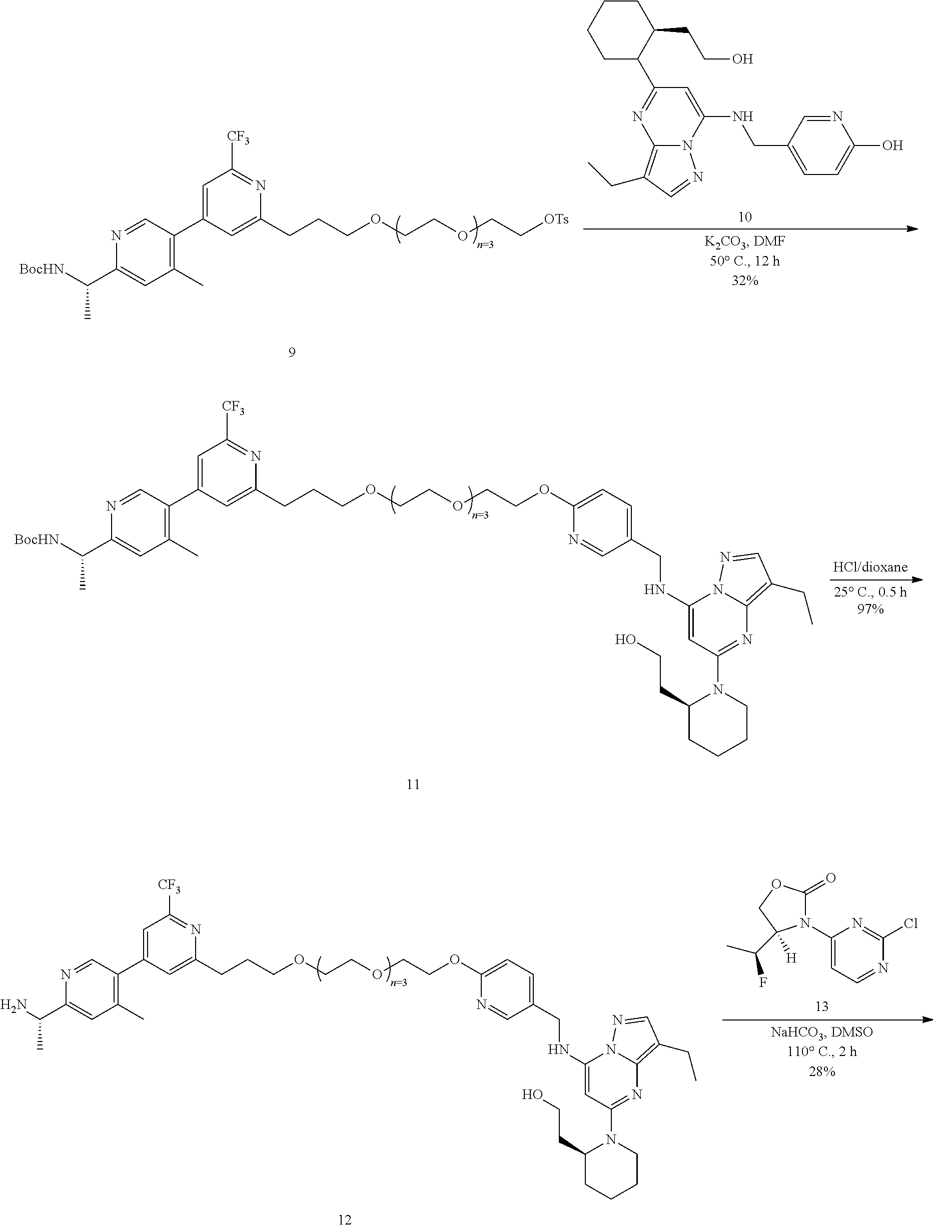

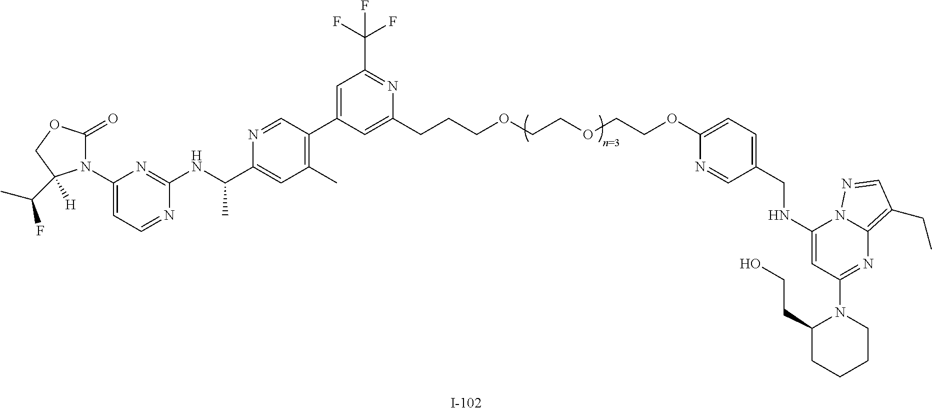

General Information: Compound 1 is described in Tetrahedron, 1998, vol. 54, #10, p. 2049-2058. Compound 2 is described in CN113248423, 2021, A. Compound 6 is described in WO2014/141104, 2014, A1.

Step 1. A mixture of tert-butyl-dimethyl-[2-[2-[2-(2-prop-2-ynoxyethoxy)ethoxy]ethoxy]ethoxy]silane (1.1 g, 3.33 mmol, 1.5 equiv), 2,4-dichloro-6-(trifluoromethyl) pyridine (480 mg, 2.22 mmol, 1.0 equiv), CuI (42 mg, 222 μmol, 0.1 equiv), dichloropalladium; triphenylphosphine (155 mg, 222 μmol, 0.1 equiv) and TEA (674 mg, 6.67 mmol, 3.0 equiv) in THF (5 mL) and ACN(10 mL), was degassed and purged with N₂ for 3 times, and the mixture was stirred at 90° C. for 16 h under N₂ atmosphere. The mixture was partitioned between H₂O 20 mL and EA 100 mL. The organic phase was separated, washed with H₂O 30 mL (10 mL*3), dried over Na₂SO₄, filtered and concentrated. The residue was purified by prep-HPLC (column: Phenomenex luna C18 150*40 mm*15 μm; mobile phase: [water (FA)-ACN]; B %: 70%-100%, 10 min) to give tert-butyl-[2-[2-[2-[2-[3-[4-chloro-6-(trifluoromethyl)-2-pyridyl]prop-2-ynoxy]ethoxy]ethoxy]ethoxy]ethoxy]-dimethyl-silane (450 mg, 38% yield). 1H NMR (400 MHZ, CDCl₃): δ 7.62-7.51 (m, 2H), 4.40 (s, 2H), 3.75-3.68 (m, 4H), 3.67-3.63 (m, 2H), 3.62 (s, 4H), 3.59 (s, 4H), 3.49 (t, J=5.4 Hz, 3H), 0.83 (s, 9H), 0.00 (s, 6H).

Step 2. To a solution of tert-butyl-[2-[2-[2-[2-[3-[4-chloro-6-(trifluoromethyl)-2-pyridyl]prop-2-ynoxy]ethoxy]ethoxy]ethoxy]ethoxy]-dimethyl-silane (370 mg, 703 μmol, 1.0 equiv) in THF (5 mL) was added PtO2 (100 mg, 440 μmol, 0.6 equiv) under N₂ atmosphere. The mixture was stirred under H₂ (15 Psi.) at 25° C. for 1 h. The reaction mixture was filtered and concentrated. The residue was purified by prep-TLC (SiO2, PE: EA=3:1) to give tert-butyl-[2-[2-[2-[2-[3-[4-chloro-6-(trifluoromethyl)-2-pyridyl]propoxy]ethoxy]ethoxy]ethoxy]ethoxy]-dimethyl-silane (160 mg, 42% yield). 1H NMR (400 MHz, CDCl₃): δ 7.53 (s, 1H), 7.41 (s, 1H), 3.78 (t, J=5.4 Hz, 2H), 3.72-3.63 (m, 10H), 3.62-3.49 (m, 6H), 2.96 (t, J=7.6 Hz, 2H), 2.14-1.97 (m, 2H), 0.91 (s, 9H), 0.08 (s, 6H).

Step 3. A mixture of tert-butyl-[2-[2-[2-[2-[3-[4-chloro-6-(trifluoromethyl)-2-pyridyl]propoxy]ethoxy]ethoxy]ethoxy]ethoxy]-dimethyl-silane (130 mg, 245 μmol, 1.0 equiv), BPD (93 mg, 367 μmol, 1.5 equiv), XPhos (23 mg, 49 μmol, 0.2 equiv), KOAc (72 mg, 735 μmol, 3.0 equiv) and Pd₂ (dba)3 (11 mg, 12. μmol, 0.05 equiv) in dioxane (5 mL) was degassed and purged with N₂ for 3 times, and then the mixture was stirred at 75° C. for 12 h under N₂ atmosphere. The reaction mixture was filtered and concentrated. The residue was purified by prep-TLC (SiO2, PE: EA=1:1) to give [2-[3-[2-[2-[2-[2-[tert-butyl(dimethyl)silyl]oxyethoxy]ethoxy]ethoxy]ethoxy]propyl]-6-(trifluoromethyl)-4-pyridyl]boronic acid (130 mg, 98% yield).

Step 4. A mixture of [2-[3-[2-[2-[2-[2-[tert-butyl(dimethyl)silyl]oxyethoxy]ethoxy]ethoxy]ethoxy]propyl]-6-(trifluoromethyl)-4-pyridyl]boronic acid (130 mg, 240 μmol, 1.0 equiv), tert-butyl N-[(1S)-1-(5-bromo-4-methyl-2-pyridyl) ethyl]carbamate (75 mg, 240 μmol, 1.0 equiv), cyclopentyl (diphenyl) phosphane; dichloromethane; dichloropalladium; iron (19 mg, 24 μmol, 0.1 equiv), Na₂CO₃ (76 mg, 722 μmol, 3.0 equiv) in dioxane (4 mL) and H₂O (0.4 mL) was degassed and purged with N₂ for 3 times, and then the mixture was stirred at 90° C. for 12 h under N₂. The mixture was partitioned between H₂O 20 mL and EA 100 mL. The organic phase was separated, washed with H₂O 30 mL (30 mL*3), dried over Na₂SO₄, filtered and concentrated. The residue was purified by prep-TLC (SiO2, Petroleum ether/Ethyl acetate=1:1) to give tert-butyl N-[(1S)-1-[5-[2-[3-[2-[2-[2-[2-[tert-butyl(dimethyl)silyl]oxyethoxy]ethoxy]ethoxy]ethoxy]propyl]-6-(trifluoromethyl)-4-pyridyl]-4-methyl-2-pyridyl]ethyl]carbamate (130 mg, 73% yield).

Step 5. To a solution of tert-butyl N-[(1S)-1-[5-[2-[3-[2-[2-[2-[2-[tert-butyl(dimethyl)silyl Joxyethoxy]ethoxy]ethoxy]ethoxy]propyl]-6-(trifluoromethyl)-4-pyridyl]-4-methyl-2-pyridyl]ethyl]carbamate (120 mg, 164 μmol, 1.0 equiv) in THF (3 mL) was added TBAF (1 M, 328 μL, 2.0 equiv). The mixture was stirred at 25° C. for 1 h. The reaction mixture was partitioned between H₂O 20 mL and EA 100 mL. The organic phase was separated, washed with H₂O 30 mL (30 mL*3), dried over Na₂SO₄, filtered and concentrated. The residue was purified by prep-TLC (SiO2, Petroleum ether/Ethyl acetate=0:1) to give tert-butyl N-[(1S)-1-[5-[2-[3-[2-[2-[2-(2-hydroxyethoxy)ethoxy]ethoxy]ethoxy]propyl]-6-(trifluoromethyl)-4-pyridyl]-4-methyl-2-pyridyl]ethyl]carbamate (100 mg, 98% yield).

Step 6. To a solution of tert-butyl N-[(1S)-1-[5-[2-[3-[2-[2-[2-(2-hydroxyethoxy) ethoxy]ethoxy]ethoxy]propyl]-6-(trifluoromethyl)-4-pyridyl]-4-methyl-2-pyridyl]ethyl]carbamate (80 mg, 130 μmol, 1 eq) in DCM (2 mL) was added TEA (39 mg, 390 μmol, 3 eq) and was added 4-methylbenzenesulfonyl chloride (50 mg, 260 μmol, 2 eq). The mixture was stirred at 25° C. for 12 h. The mixture was partitioned between H₂O 20 mL and EA 100 mL. The organic phase was separated, washed with H₂O 30 mL (30 mL*3), dried over Na₂SO₄, filtered and concentrated. The residue was purified by prep-TLC (SiO2, Petroleum ether/Ethyl acetate=2:1) to give 2-[2-[2-[2-[3-[4-[6-[(1S)-1-(tert-butoxycarbonylamino) ethyl]-4-methyl-3-pyridyl]-6-(trifluoromethyl)-2-pyridyl]propoxy]ethoxy]ethoxy]ethoxy]ethyl 4-methylbenzenesulfonate (60 mg, 59% yield).

Step 7. To a solution of 2-[2-[2-[2-[3-[4-[6-[(1S)-1-(tert-butoxycarbonylamino) ethyl]-4-methyl-3-pyridyl]-6-(trifluoromethyl)-2-pyridyl]propoxy]ethoxy]ethoxy]ethoxy]ethyl 4-methylbenzenesulfonate (60 mg, 77 μmol, 1.0 equiv) and 5-[[[3-ethyl-5-[(2S)-2-(2-hydroxyethyl)-1-piperidyl]pyrazolo[1,5-a]pyrimidin-7-yl]amino]methyl]pyridin-2-ol (30 mg, 77 μmol, 1.0 equiv) in DMF (1 mL) was added K₂CO₃ (32 mg, 233 μmol, 3.0 equiv). The mixture was stirred at 50° C. for 12 h. The reaction mixture filtered and concentrated. The residue was purified by prep-HPLC (column: Unisil 3-100 C18 Ultra 150*50 mm*3 μm; mobile phase: [water (FA)-ACN]; B %: 39%-69%,7 min) to give tert-butyl N-[(1S)-1-[5-[2-[3-[2-[2-[2-[2-[5-[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]propyl]-6-(trifluoromethyl)-4-pyridyl]-4-methyl-2-pyridyl]ethyl]carbamate (25 mg, 32% yield).

Step 8. To a solution of tert-butyl N-[(1S)-1-[5-[2-[3-[2-[2-[2-[2-[5-[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]propyl]-6-(trifluoromethyl)-4-pyridyl]-4-methyl-2-pyridyl]ethyl]carbamate (25 mg, 25 μmol, 1.0 equiv) in DCM (1 mL) was added HCl/dioxane (4 M, 0.5 mL, 79 equiv). The mixture was stirred at 25° C. for 0.5 h. The reaction mixture filtered and concentrated. The residue was used for next step directly to give 2-[(2S)-1-[7-[[6-[2-[2-[2-[2-[3-[4-[6-[(1S)-1-aminoethyl]-4-methyl-3-pyridyl]-6-(trifluoromethyl)-2-pyridyl]propoxy]ethoxy]ethoxy]ethoxy]ethoxy]-3-pyridyl]methylamino]-3-ethyl-pyrazolo[1,5-a]pyrimidin-5-yl]-2-piperidyl]ethanol (22 mg, 97% yield).

Step 9. To a solution of 2-[(2S)-1-[7-[[6-[2-[2-[2-[2-[3-[4-[6-[(1S)-1-aminoethyl]-4-methyl-3-pyridyl]-6-(trifluoromethyl)-2-pyridyl]propoxy]ethoxy]ethoxy]ethoxy]ethoxy]-3-pyridyl]methylamino]-3-ethyl-pyrazolo[1,5-a]pyrimidin-5-yl]-2-piperidyl]ethanol (22 mg, 24 μmol, 1.0 equiv) and (4R)-3-(2-chloropyrimidin-4-yl)-4-[(1S)-1-fluoroethyl]oxazolidin-2-one (9 mg, 36 μmol, 1.5 equiv) in DMSO (0.5 mL) was added NaHCO₃ (10 mg, 123 μmol, 5.0 equiv). The mixture was stirred at 110° C. for 2 h. The reaction mixture filtered and concentrated. The residue was purified by prep-HPLC (column: Waters Xbridge C18 150*50 mm*10 μm; mobile phase: [water (NH₄HCO₃)-ACN]; B %: 56%-86%,10 min) to give (4R)-3-[2-[[(1S)-1-[5-[2-[3-[2-[2-[2-[2-[5-[[[3-ethyl-5-[(2S)-2-(2-hydroxyethyl)-1-piperidyl]pyrazolo[1,5-a]pyrimidin-7-yl]amino]methyl]-2-pyridyl]oxy]ethoxy]ethoxy]ethoxy]ethoxy]propyl]-6-(trifluoromethyl)-4-pyridyl]-4-methyl-2-pyridyl]ethyl]amino]pyrimidin-4-yl]-4-[(1S)-1-fluoroethyl]oxazolidin-2-one (8 mg, 28% yield). ¹H NMR (400 MHZ, DMSO): δ 8.43 (s, 1H), 8.22 (d, J=2.1 Hz, 2H), 7.97-7.79 (m, 2H), 7.77-7.75 (m, 2H), 7.65 (s, 2H), 7.35 (s, 1H), 7.25 (d, J=5.4 Hz, 1H), 6.78 (d, J=8.7 Hz, 1H), 5.59 (s, 1H), 5.11-4.93 (m, 1H), 4.83-4.64 (m, 2H), 4.63-4.39 (m, 6H), 4.35-4.25 (m, 3H), 3.71-3.67 (m, 2H), 3.54-3.51 (m, 2H), 3.50-3.47 (m, 4H), 3.46-3.42 (m, 4H), 2.91 (t, J=7.9 Hz, 3H), 2.84-2.73 (m, 2H), 2.27 (s, 4H), 2.01-1.89 (m, 3H), 1.87-1.73 (m, 2H), 1.69-1.60 (m, 4H), 1.57 (s, 2H), 1.48 (d, J=7.2 Hz, 4H), 1.44-1.28 (m, 3H), 1.24 (s, 1H), 1.16 (t, J=7.5 Hz, 5H). LC-MS: MS (ES⁺): RT=2.590 min, m/z=1103.5 [M+H⁺]; LCMS method: 10.

### Example 81—the Synthetic Route for I-103

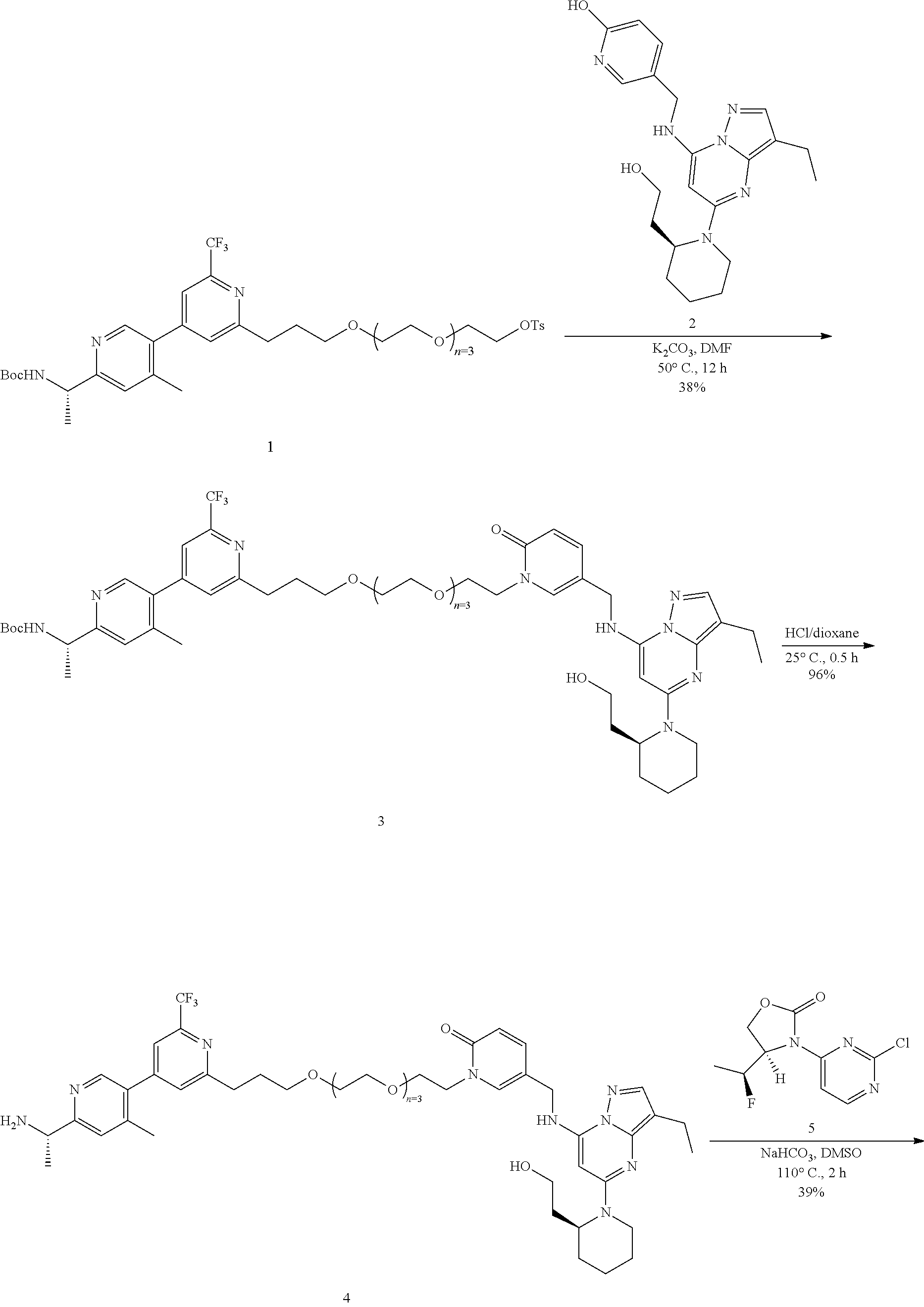

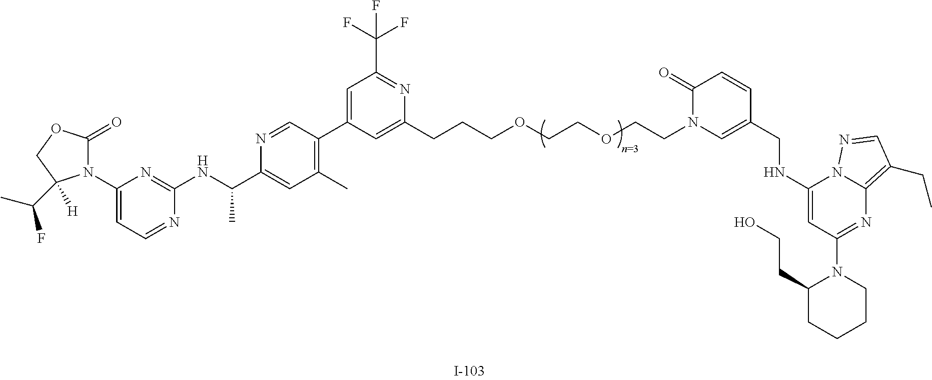

Step 1. To a solution of 2-[2-[2-[2-[3-[4-[6-[(1S)-1-(tert-butoxycarbonylamino) ethyl]-4-methyl-3-pyridyl]-6-(trifluoromethyl)-2-pyridyl]propoxy]ethoxy]ethoxy]ethoxy]ethyl 4-methylbenzenesulfonate (60 mg, 77 μmol, 1.0 equiv) and 5-[[[3-ethyl-5-[(2S)-2-(2-hydroxyethyl)-1-piperidyl]pyrazolo[1,5-a]pyrimidin-7-yl]amino]methyl]pyridin-2-ol (30 mg, 77 μmol, 1.0 equiv) in DMF (1 mL) was added K₂CO₃ (32 mg, 233 μmol, 3.0 equiv). The mixture was stirred at 50° C. for 12 h. The residue was purified by prep-HPLC (column: Unisil 3-100 C18 Ultra 150*50 mm*3 μm; mobile phase: [water (FA)-ACN]; B %: 39%-69%,7 min) to give tert-butyl N-[(1S)-1-[5-[2-[3-[2-[2-[2-[2-[5-[[[3-ethyl-5-[(2S)-2-(2-hydroxyethyl)-1-piperidyl]pyrazolo[1,5-a]pyrimidin-7-yl]amino]methyl]-2-oxo-1-pyridyl]ethoxy]ethoxy]ethoxy]ethoxy]propyl]-6-(trifluoromethyl)-4-pyridyl]-4-methyl-2-pyridyl]ethyl]carbamate (30 mg, 38% yield).

Step 2. To a solution of tert-butyl N-[(1S)-1-[5-[2-[3-[2-[2-[2-[2-[5-[[[3-ethyl-5-[(2S)-2-(2-hydroxyethyl)-1-piperidyl]pyrazolo[1,5-a]pyrimidin-7-yl]amino]methyl]-2-oxo-1-pyridyl]ethoxy]ethoxy]ethoxy]ethoxy]propyl]-6-(trifluoromethyl)-4-pyridyl]-4-methyl-2-pyridyl]ethyl]carbamate (30 mg, 30 μmol, 1.0 equiv) in DCM (1 mL) was added HCl/dioxane (4 M, 0.5 mL, 66 equiv). The mixture was stirred at 25° C. for 0.5 h. The reaction mixture filtered and concentrated. The residue was used for next step directly to give 1-[2-[2-[2-[2-[3-[4-[6-[(1S)-1-aminoethyl]-4-methyl-3-pyridyl]-6-(trifluoromethyl)-2-pyridyl]propoxy]ethoxy]ethoxy]ethoxy]ethyl]-5-[[[3-ethyl-5-[(2S)-2-(2-hydroxyethyl)-1-piperidyl]pyrazolo[1,5-a]pyrimidin-7-yl]amino]methyl]pyridin-2-one (26 mg, 96% yield).

Step 3. To a solution of 1-[2-[2-[2-[2-[3-[4-[6-[(1S)-1-aminoethyl]-4-methyl-3-pyridyl]-6-(trifluoromethyl)-2-pyridyl]propoxy]ethoxy]ethoxy]ethoxy]ethyl]-5-[[3-ethyl-5-[(2S)-2-(2-hydroxyethyl)-1-piperidyl]pyrazolo[1,5-a]pyrimidin-7-yl]amino]methyl]pyridin-2-one (26 mg, 29 μmol, 1.0 equiv) and (4R)-3-(2-chloropyrimidin-4-yl)-4-[(1S)-1-fluoroethyl]oxazolidin-2-one (10 mg, 43 μmol, 1.5 equiv) in DMSO (0.5 mL) was added NaHCO₃ (12 mg, 145 μmol, 5.0 equiv). The mixture was stirred at 110° C. for 2 h. The reaction mixture filtered and concentrated. The residue was purified by prep-HPLC (column: Waters Xbridge C18 150*50 mm*10 μm; mobile phase: [water (NH₄HCO₃)-ACN]; B %: 52%-82%, 10 min) to give compound (4R)-3-[2-[(1S)-1-[5-[2-[3-[2-[2-[2-[2-[5-[[[3-ethyl-5-[(2S)-2-(2-hydroxyethyl)-1-piperidyl]pyrazolo[1,5-a]pyrimidin-7-yl]amino]methyl]-2-oxo-1-pyridyl]ethoxy]ethoxy]ethoxy]ethoxy]propyl]-6-(trifluoromethyl)-4-pyridyl]-4-methyl-2-pyridyl]ethyl]amino]pyrimidin-4-yl]-4-[(1S)-1-fluoroethyl]oxazolidin-2-one (13 mg, 39% yield). ¹H NMR (400 MHZ, DMSO): δ 8.43 (s, 1H), 8.31-8.15 (m, 1H), 7.97-7.83 (m, 1H), 7.82-7.66 (m, 4H), 7.64 (s, 2H), 7.53-7.50 (m, 1H), 7.36 (s, 1H), 7.25 (d, J=5.5 Hz, 1H), 6.37 (d, J=9.4 Hz, 1H), 5.57 (s, 1H), 5.12-4.94 (m, 1H), 4.79-4.66 (m, 2H), 4.64-4.50 (m, 2H), 4.49-4.40 (m, 2H), 4.35-4.28 (m, 1H), 4.28-4.23 (m, 2H), 3.99-3.96 (m, 2H), 3.63-3.59 (m, 2H), 3.47-3.43 (m, 9H), 3.38 (d, J=4.9 Hz, 6H), 2.94-2.76 (m, 3H), 2.27 (s, 4H), 2.00-1.76 (m, 4H), 1.70-1.56 (m, 6H), 1.48 (d, J=7.2 Hz, 3H), 1.43-1.31 (m, 2H), 1.16 (t, J=7.5 Hz, 5H). LC-MS: MS (ES⁺): RT=2.513 min, m/z=1103.5 [M+H⁺]; LCMS method: 10.

### Example 82—the Synthetic Route for I-104

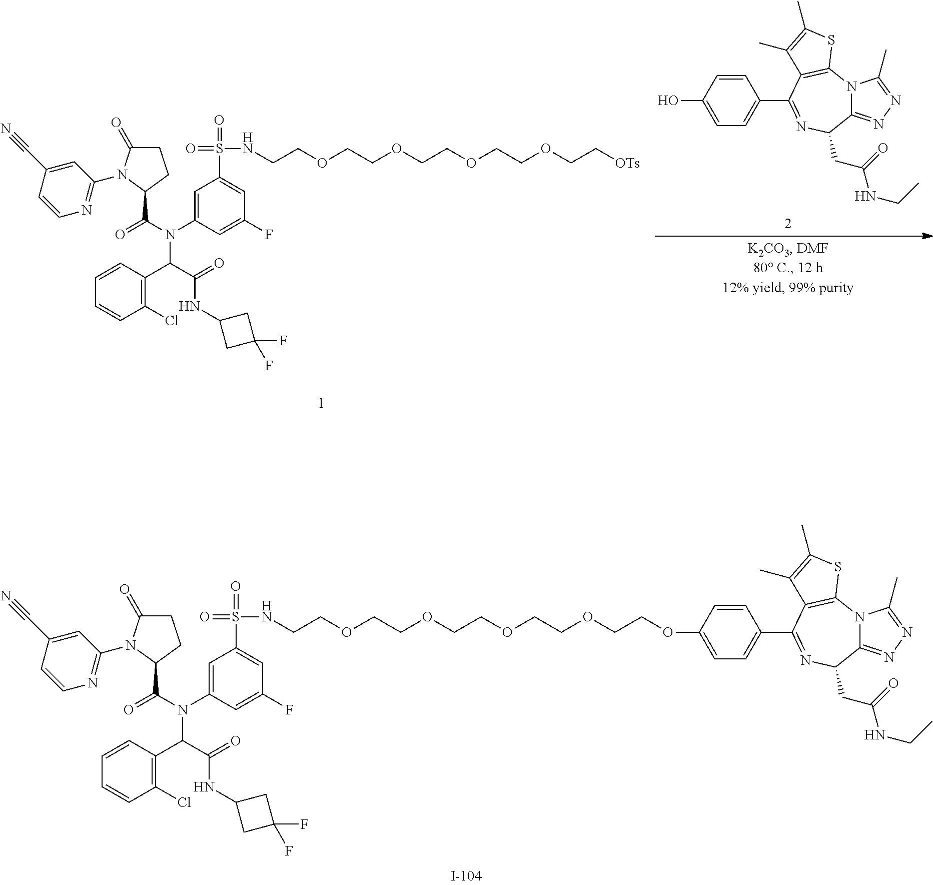 [SMILES: Cc1ccc(S(=O)(=O)OCCOCCOCCOCCOCCNS(=O)(=O)c2cc(F)cc(N(C(=O)[C@@H]3CCC(=O)N3c3cc(C#N)ccn3)C(C(=O)NC3CC(F)(F)C3)c3ccccc3Cl)c2)cc1 ; CCNC(=O)C[C@@H]1N=C(c2ccc(O)cc2)c2c(sc(C)c2C)-n2c(C)nnc21 ; CCNC(=O)C[C@@H]1N=C(c2ccc(OCCOCCOCCOCCOCCNS(=O)(=O)c3cc(F)cc(N(C(=O)[C@@H]4CCC(=O)N4c4cc(C#N)ccn4)C(C(=O)NC4CC(F)(F)C4)c4ccccc4Cl)c3)cc2)c2c(sc(C)c2C)-n2c(C)nnc21]

Step 1. A mixture of 2-[2-[2-[2-[2-[[3-[[1-(2-chlorophenyl)-2-[(3,3-difluoro cyclobutyl)amino]-2-oxo-ethyl]-[(2S)-1-(4-cyano-2-pyridyl)-5-oxo-pyrrolidine-2-carbonyl]amino]-5-fluoro-phenyl]sulfonylamino]ethoxy]ethoxy]ethoxy]ethoxy]ethyl 4-methylbenzenesulfonate (98 mg, 94 μmol, 1.0 equiv), N-ethyl-2-[(9S)-7-(4-hydroxyphenyl)-4,5,13-trimethyl-3-thia-1,8,11,12-tetrazatricyclo[8.3.0.0^{2,6}]trideca-2 (6),4,7,10,12-pentaen-9-yl]acetamide (135 mg, 331 μmol, 3.5 equiv), K₂CO₃ (39 mg, 283 μmol, 3.0 equiv) in CH₃CN (4 mL) was degassed and purged with N₂ for 3 times, and then the mixture was stirred at 80° C. for 12 h under N₂ atmosphere. The reaction mixture was filtered. The residue was purified by prep-TLC (SiO2, DCM: MeOH=10:1). Then purified by prep-HPLC (column: Phenomenex luna C18 150*25 mm*10 μm; mobile phase: [water (FA)-ACN]; B %: 40%-70%, 8 min) to give the (2S)—N-[1-(2-chlorophenyl)-2-[(3,3-difluorocyclobutyl)amino]-2-oxo-ethyl]-1-(4-cyano-2-pyridyl)-N-[3-[2-[2-[2-[2-[2-[4-[(9S)-9-[2-(ethylamino)-2-oxo-ethyl]-4,5, 13-trimethyl-3-thia-1,8,11,12-tetrazatricyclo[8.3.0.0^{2,6}]trideca-2 (6),4,7,10,12-pentaen-7-yl]phenoxy]ethoxy]ethoxy]ethoxy]ethoxy]ethylsulfamoyl]-5-fluoro-phenyl]-5-oxo-pyrrolidine-2-carboxamide (15 mg, 12 μmol, 12% yield, 99% purity) as a off-white solid. 1H NMR: (400 MHZ, MeOD) δ=8.83-8.56 (m, 2H), 8.05-7.83 (m, 1H), 7.74 (s, 1H), 7.66-7.58 (m, 2H), 7.52-7.35 (m, 3H), 7.29-7.16 (m, 1H), 7.13-7.06 (m, 1H), 7.03-6.96 (m, 1H), 6.59-6.38 (m, 2H), 5.57-5.50 (m, 1H), 4.71-4.56 (m, 1H), 4.42-4.35 (m, 2H), 4.29-4.06 (m, 4H), 3.77-3.71 (m, 2H), 3.59-3.37 (m, 16H), 3.35 (d, J=4.2 Hz, 2H), 3.05-2.71 (m, 6H), 2.66-2.33 (m, 6H), 2.23-2.03 (m, 3H), 1.76-1.48 (m, 7H), 1.26-1.17 (m, 3H). LC-MS: MS (ES⁺): RT=2.843 min, m/z=1259.7 [M+H⁺]; LCMS method 15.

### Example 83—the Synthetic Route for I-105

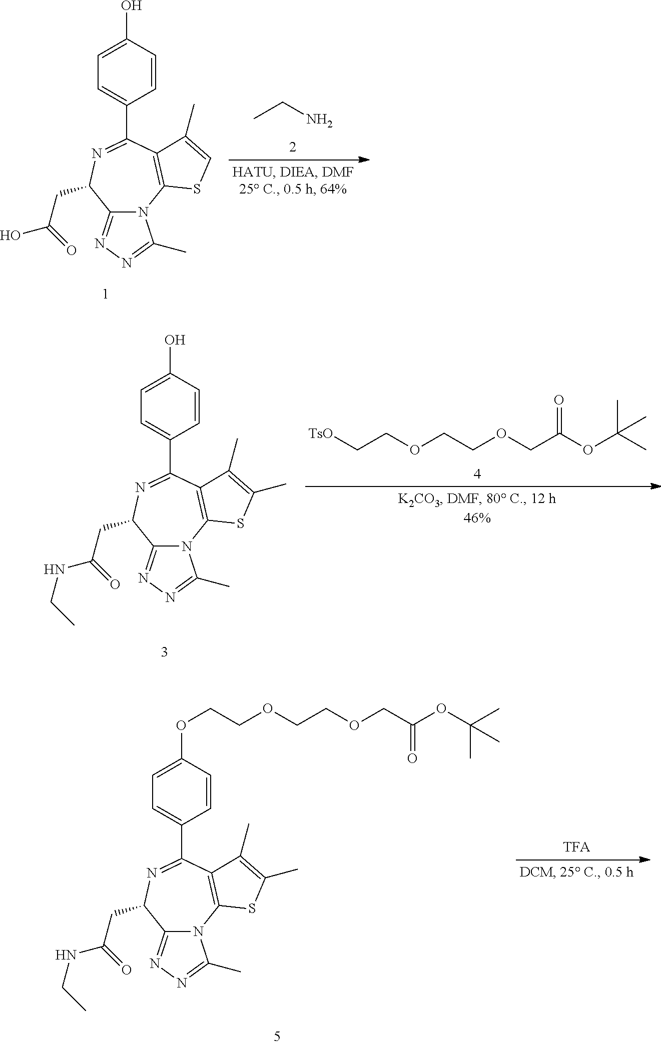

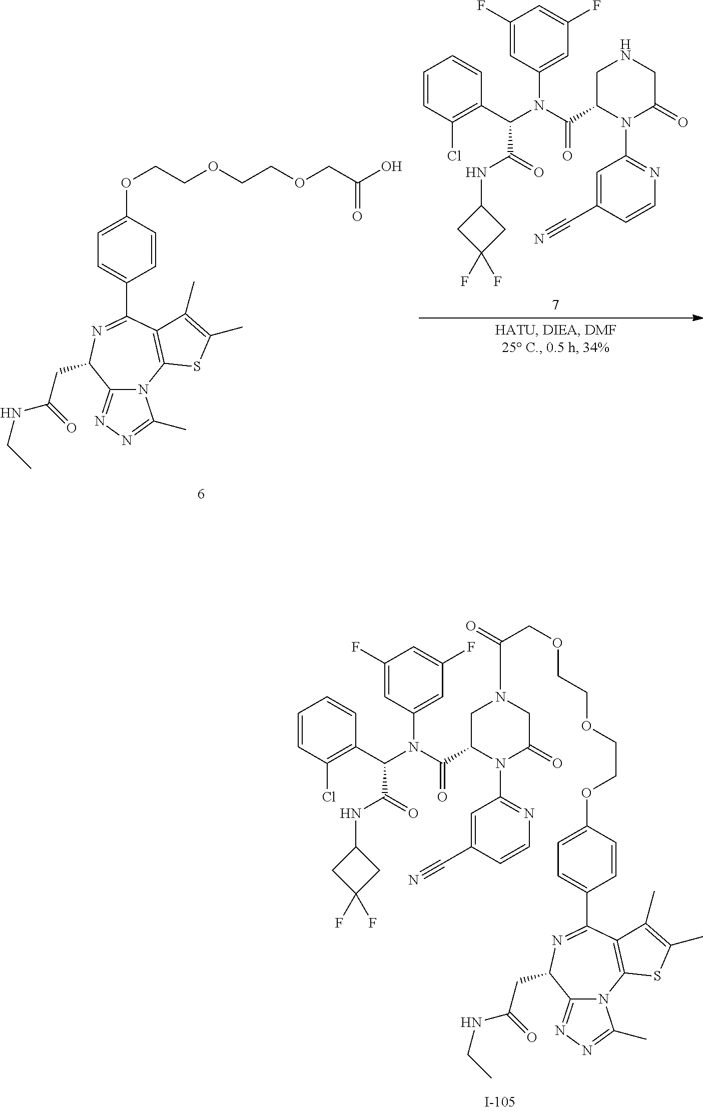 [SMILES: CCNC(=O)C[C@@H]1N=C(c2ccc(OCCOCCOCC(=O)O)cc2)c2c(sc(C)c2C)-n2c(C)nnc21 ; N#Cc1ccnc(N2C(=O)CNC[C@H]2C(=O)N(c2cc(F)cc(F)c2)[C@H](C(=O)NC2CC(F)(F)C2)c2ccccc2Cl)c1 ; CCNC(=O)C[C@@H]1N=C(c2ccc(OCCOCCOCC(=O)N3CC(=O)N(c4cc(C#N)ccn4)[C@H](C(=O)N(c4cc(F)cc(F)c4)[C@H](C(=O)NC4CC(F)(F)C4)c4ccccc4Cl)C3)cc2)c2c(sc(C)c2C)-n2c(C)nnc21]

General Information: Compound 7 is described in WO2013/107291, 2013, A^{1.}

Step 1. To a solution of 2-[(9S)-7-(4-hydroxyphenyl)-4,5,13-trimethyl-3-thia-1,8,11,12-tetrazatricyclo[8.3.0.0^{2,6}]trideca-2 (6),4,7, 10, 12-pentaen-9-yl]acetic acid (700 mg, 1.83 mmol, 1.0 equiv) and ethanamine (82.5 mg, 1.83 mmol, 1.0 equiv) in DMF (5 mL) was added DIEA (710 mg, 5.49 mmol, 3.0 equiv) and HATU (1.04 g, 2.75 mmol, 1.5 equiv). The mixture was stirred at 25° C. for 0.5 h. The residue was purified by prep-HPLC (column: Phenomenex luna C18 150*40 mm*15 μm; mobile phase: [water (FA)-ACN]; B %: 17%-47%,10 min) to afford N-ethyl-2-[(9S)-7-(4-hydroxyphenyl)-4,5,13-trimethyl-3-thia-1,8,11,12-tetrazatricyclo[8.3.0.0^{2,6}]trideca-2 (6),4,7, 10, 12-pentaen-9-yl]acetamide (480 mg, 1.17 mmol, 64% yield) as a yellow solid.

Step 2. To a solution of N-ethyl-2-[(9S)-7-(4-hydroxyphenyl)-4,5,13-trimethyl-3-thia-1,8, 11,12-tetrazatricyclo[8.3.0.0^{2,6}]trideca-2 (6),4,7, 10, 12-pentaen-9-yl]acetamide (220 mg, 537 μmol, 1.0 equiv) and tert-butyl 2-[2-[2-(p-tolylsulfonyloxy)ethoxy]ethoxy]acetate (302 mg, 806 μmol, 1.5 equiv) in DMF (5 mL) was added K₂CO₃ (223 mg, 1.61 mmol, 3.0 equiv) and NaI (80.5 mg, 537 μmol, 1.0 equiv). The mixture was stirred at 80° C. for 12 h. The residue was filtered to remove K₂CO₃ and then concentrated to afford crude product. The residue was purified by prep-HPLC (column: Waters Xbridge 150*25 mm*5 μm; mobile phase: [water (NH₄HCO₃)-ACN]; B %: 34%-64%,8 min) to afforded tert-butyl 2-[2-[2-[4-[(9S)-9-[2-(ethylamino)-2-oxo-ethyl]-4,5,13-trimethyl-3-thia-1,8,11,12-tetrazatricyclo[8.3.0.0^{2,6}]trideca-2 (6),4,7,10,12-pentaen-7-yl]phenoxy]ethoxy]ethoxy]acetate (150 mg, 245 μmol, 46% yield) as a yellow oil.

Step 3. To a solution of tert-butyl 2-[2-[2-[4-[(9S)-9-[2-(ethylamino)-2-oxo-ethyl]-4,5,13-trimethyl-3-thia-1,8, 11,12-tetrazatricyclo[8.3.0.0^{2,6}]trideca-2 (6),4,7,10,12-pentaen-7-yl]phenoxy]ethoxy]ethoxy]acetate (75.0 mg, 123 μmol, 1.0 equiv) in DCM (2 mL) was added TFA (1 mL). The mixture was stirred at 25° C. for 0.5 h. The reaction mixture was concentrated to afford crude product. 2-[2-[2-[4-[(9S)-9-[2-(ethylamino)-2-oxo-ethyl]-4,5,13-trimethyl-3-thia-1,8,11,12-tetrazatricyclo[8.3.0.0^{2,6}]trideca-2 (6),4, 7, 10,12-pentaen-7-yl]phenoxy]ethoxy]ethoxy]acetic acid (71 mg, TFA salt) was used into the next step without further purification.

Step 4. To a solution of 2-[2-[2-[4-[(9S)-9-[2-(ethylamino)-2-oxo-ethyl]-4,5,13-trimethyl-3-thia-1,8,11,12-tetrazatricyclo[8.3.0.0^{2,6}]trideca-2 (6) ,4,7, 10,12-pentaen-7-yl]phenoxy]ethoxy]ethoxy]acetic acid (63.0 mg, 113 μmol, 1.0 equiv) and (2S)—N-[(1S)-1-(2-chlorophenyl)-2-[(3,3-difluorocyclobutyl)amino]-2-oxo-ethyl]-1-(4-cyano-2-pyridyl)-N-(3,5-difluorophenyl)-6-oxo-piperazine-2-carboxamide (82.7 mg, 113 μmol, 1.0 equiv, TFA) in DMF (2 mL) was added DIEA (29.3 mg, 226 μmol, 2.0 equiv) and HATU (64.8 mg, 170 μmol, 1.5 equiv). The mixture was stirred at 25° C. for 0.5 h. The residue was purified by prep-HPLC (column: Phenomenex C18 75*30 mm*3 μm; mobile phase: [water (FA)-ACN]; B %: 48%-78%,7 min) to afford (2S)—N-[(1S)-1-(2-chlorophenyl)-2-[(3,3-difluorocyclobutyl)amino]-2-oxo-ethyl]-1-(4-cyano-2-pyridyl)-N-(3,5-difluorophenyl)-4-[2-[2-[2-[4-[(9S)-9-[2-(ethylamino)-2-oxo-ethyl]-4,5,13-trimethyl-3-thia-1,8,11,12-tetrazatricyclo[8.3.0.0^{2,6}]trideca-2 (6),4,7,10,12-pentaen-7-yl]phenoxy]ethoxy]ethoxy]acetyl]-6-oxo-piperazine-2-carboxamide (66.4 mg, 57.0 μmol, 50% yield, 99% purity) as a yellow solid. ¹H NMR (400 MHZ, CD₃OD) δ 8.88-8.55 (m, 2H), 8.29-7.75 (m, 2H), 7.49-7.29 (m, 3H), 7.28-7.17 (m, 2H), 7.16-6.68 (m, 6H), 6.50 (s, 1H), 4.91 (m, 2H), 4.85-4.48 (m, 3H), 4.47-4.24 (m, 2H), 4.23-4.01 (m, 3H), 4.00-3.76 (m, 4H), 3.75-3.67 (m, 3H), 3.45-3.36 (m, 1H), 3.28-3.21 (m, 2H), 2.94-2.68 (m, 5H), 2.56-2.29 (m, 5H), 1.75-1.58 (m, 3H), 1.22-1.14 (m, 3H). LC-MS: (ES⁺): RT =2.508 min, m/z=1152.3 [M+H⁺]; LCMS method 25.

### Example 84—the Synthetic Route for I-106

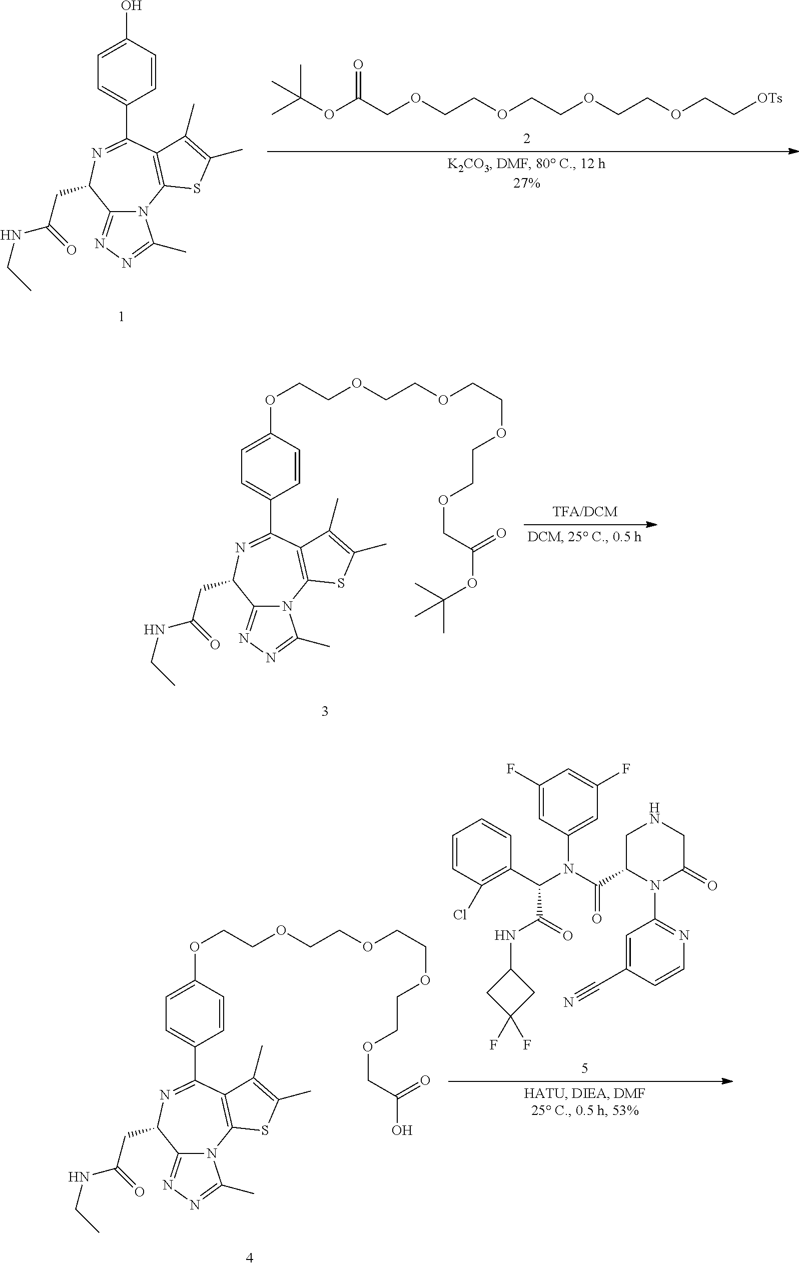

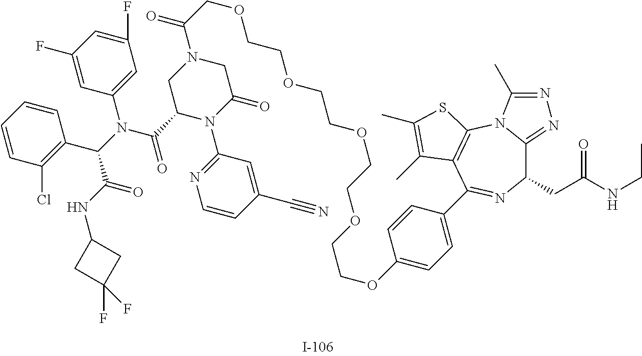 [SMILES: CCNC(=O)C[C@@H]1N=C(c2ccc(OCCOCCOCCOCCOCC(=O)N3CC(=O)N(c4cc(C#N)ccn4)[C@H](C(=O)N(c4cc(F)cc(F)c4)[C@H](C(=O)NC4CC(F)(F)C4)c4ccccc4Cl)C3)cc2)c2c(sc(C)c2C)-n2c(C)nnc21]

Step 1. To a solution of N-ethyl-2-[(9S)-7-(4-hydroxyphenyl)-4,5,13-trimethyl-3-thia-1,8,11,12-tetrazatricyclo[8.3.0.0^{2,6}]trideca-2 (6),4,7,10,12-pentaen-9-yl]acetamide (220 mg, 537 μmol, 1.0 equiv) and tert-butyl 2-[2-[2-[2-[2-(p-tolylsulfonyloxy)ethoxy]ethoxy]ethoxy]ethoxy]acetate (372 mg, 806 μmol, 1.5 equiv) in DMF (2 mL) was added K₂CO₃ (223 mg, 1.61 mmol, 3.0 equiv) and NaI (80.5 mg, 537 μmol, 1.0 equiv). The mixture was stirred at 80° C. for 12 h. The residue was filtered to remove K₂CO₃ and then concentrated to afford crude product. The residue was purified by prep-HPLC (column: Waters Xbridge 150*25 mm*5 μm; mobile phase: [water (NH₄HCO₃)-ACN]; B %: 34%-64%,8 min) to afford tert-butyl 2-[2-[2-[2-[2-[4-[(9S)-9-[2-(ethylamino)-2-oxo-ethyl]-4,5,13-trimethyl-3-thia-1,8,11,12-tetrazatricyclo[8.3.0.0^{2,6}]trideca-2 (6),4,7, 10,12-pentaen-7-yl]phenoxy]ethoxy]ethoxy]ethoxy]ethoxy]acetate (100 mg, 143 μmol, 27% yield) as a yellow oil.

Step 2. To a solution of tert-butyl 2-[2-[2-[2-[2-[4-[(9S)-9-[2-(ethylamino)-2-oxo-ethyl]-4,5,13-trimethyl-3-thia-1,8,11,12-tetrazatricyclo[8.3.0.0^{2,6}]trideca-2 (6),4,7, 10,12-pentaen-7-yl]phenoxy]ethoxy]ethoxy]ethoxy]ethoxy]acetate (80.0 mg, 114 μmol, 1.0 equiv) in DCM (2 mL) was added TFA (1 mL). The mixture was stirred at 25° C. for 0.5 h. The reaction mixture was concentrated to afford crude product. 2-[2-[2-[2-[2-[4-[(9S)-9-[2-(ethylamino)-2-oxo-ethyl]-4,5,13-trimethyl-3-thia1,8,11,12-tetrazatricyclo[8.3.0.0^{2,6}]trideca-2 (6),4,7,10,12-pentaen-7-yl]phenoxy]ethoxy]ethoxy]ethoxy]ethoxy]acetic acid (76.0 mg, TFA salt) was used into the next step without further purification.

Step 3. To a solution of 2-[2-[2-[2-[2-[4-[(9S)-9-[2-(ethylamino)-2-oxo-ethyl]-4,5,13-trimethyl-3-thia-1,8,11,12-tetrazatricyclo[8.3.0.0^{2,6}]trideca-2 (6),4,7, 10,12-pentaen-7-yl]phenoxy]ethoxy]ethoxy]ethoxy]ethoxy]acetic acid (68.0 mg, 106 μmol, 1.0 equiv) and (2S)—N-[(1S)-1-(2-chlorophenyl)-2-[(3,3-difluorocyclobutyl)amino]-2-oxo-ethyl]-1-(4-cyano-2-pyridyl)-N-(3,5-difluorophenyl)-6-oxo-piperazine-2-carboxamide (77.0 mg, 106 μmol, 1.0 equiv, TFA salt) in DMF (2 mL) was added DIEA (27.3 mg, 211 μmol, 2.0 equiv) and HATU (60.3 mg, 158 μmol, 1.5 equiv). The mixture was stirred at 25° C. for 0.5 h. The residue was purified by prep-HPLC (column: Phenomenex C18 75*30 mm*3 μm; mobile phase: [water (FA)-ACN]; B %: 48%-78%,7 min) to afford (2S)—N-[(1S)-1-(2-chlorophenyl)-2-[(3,3-difluorocyclobutyl)amino]-2-oxo-ethyl]-1-(4-cyano-2-pyridyl)-N-(3,5-difluorophenyl)-4-[2-[2-[2-[2-[2-[4-[(9S)-9-[2-(ethylamino)-2-oxo-ethyl]-4,5,13-trimethyl-3-thia-1,8,11,12-tetrazatricyclo[8.3.0.0^{2,6}]trideca-2 (6),4,7, 10,12-pentaen-7-yl]phenoxy]ethoxy]ethoxy]ethoxy]ethoxy]acetyl]-6-oxo-piperazine-2-carboxamide (70.4 mg, 56.2 μmol, 53% yield, 99% purity) as a yellow solid. ¹H NMR (400 MHZ, CD₃OD) δ 8.88-8.61 (m, 2H), 8.39-8.27 (m, 1H), 8.10-7.77 (m, 1H), 7.55-7.37 (m, 4H), 7.30-7.20 (m, 1H), 7.15-7.02 (m, 2H), 7.00-6.71 (m, 4H), 6.54-6.26 (m, 1H), 5.18-4.97 (m, 1H), 4.84 (s, 3H), 4.45-4.28 (m, 2H), 4.25-3.97 (m, 4H), 3.88-3.75 (m, 3H), 3.72-3.57 (m, 12H), 3.45-3.37 (m, 1H), 3.30-3.25 (m, 2H), 2.95-2.67 (m, 5H), 2.37 (m, 5H), 1.75-1.69 (m, 3H), 1.21-1.16 (m, 3H). LC-MS: (ES⁺): RT=2.944 min, m/z=1240.4 [M+H⁺]; LCMS method 10.

### Example 85—the Synthetic Route for I-107

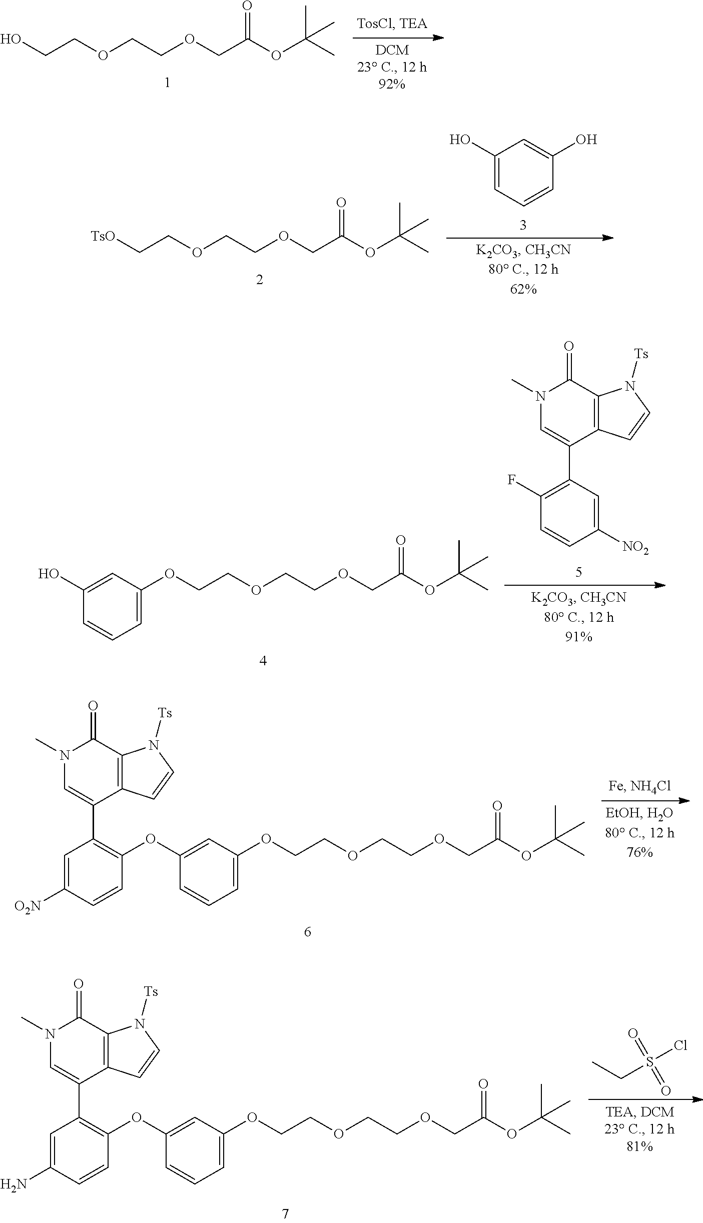

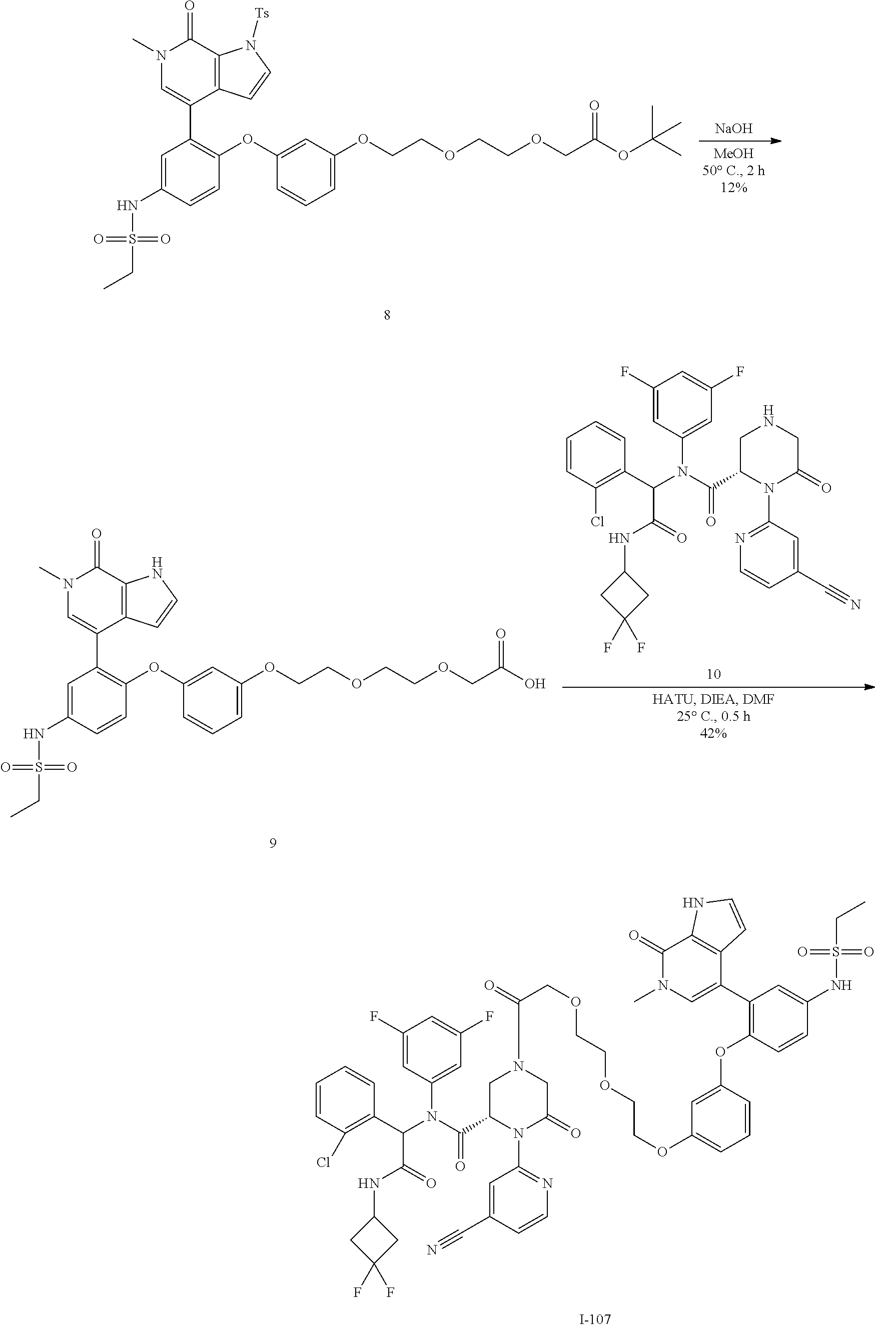

General Information: Synthetic route for compound 5 is described in WO2013/97601, 2013, A¹. Synthetic route for compound 10 is described in WO2013/107291, 2013, A¹

Step 1. A mixture of tert-butyl 2-[2-(2-hydroxyethoxy)ethoxy]acetate (2 g, 9.08 mmol, 1.0 equiv), TosCl (2.60 g, 13.6 mmol, 1.5 equiv), TEA (1.84 g, 18.2 mmol, 2.5 mL, 2.0 equiv) in DCM (20 mL) was stirred at 23° C. for 12 h. The mixture was filtered and concentrated under reduced pressure to give a residue. The residue was purified by column chromatography (SiO₂, Petroleum ether/Ethyl acetate=10/1 to 6/1) to give the desired product. Compound tert-butyl 2-[2-[2-(p-tolylsulfonyloxy)ethoxy]ethoxy]acetate (3.14 g, 8.39 mmol, 92% yield) was obtained as a yellow oil.

Step 2. A mixture of tert-butyl 2-[2-[2-(p-tolylsulfonyloxy)ethoxy]ethoxy]acetate (2.6 g, 6.9 mmol, 1.0 equiv), benzene-1,3-diol (2.29 g, 20.8 mmol, 3.48 mL, 3.0 equiv), K₂CO₃ (2.88 g, 20.83 mmol, 3 equiv) in CH₃CN(30 mL) was degassed and purged with N₂ for 3 times, and then the mixture was stirred at 80° C. for 12 h under N₂ atmosphere. The mixture was filtered and was concentrated to give the residue. The residue was purified by prep-HPLC (FA condition; column: YMC Triart C18 250*50 mm*7 μm; mobile phase: [water (FA)-ACN]; B %: 25%-55%,23 min). Compound tert-butyl 2-[2-[2-(3-hydroxyphenoxy)ethoxy]ethoxy]acetate (1.35 g, 4.32 mmol, 62% yield) was obtained as a yellow oil.

Step 3. A mixture of tert-butyl 2-[2-[2-(3-hydroxyphenoxy)ethoxy]ethoxy]acetate (212 mg, 679 μmol, 1.0 equiv), 4-(2-fluoro-5-nitro-phenyl)-6-methyl-1-(p-tolylsulfonyl) pyrrolo[2,3-c]pyridin-7-one (300 mg, 679 μmol, 1.0 equiv), K₂CO₃ (281.78 mg, 2.04 mmol, 3.0 equiv) in CH₃CN(5 mL) was degassed and purged with N₂ for 3 times, and then the mixture was stirred at 80° C. for 12 h under N₂ atmosphere. The mixture was filtered and concentrated to give the residue. The crude product was purified by chromatography on a silica gel (petroleum ether: ethyl acetate=10/1 to 3/1) to give tert-butyl 2-[2-[2-[3-[2-[6-methyl-7-oxo-1-(p-tolylsulfonyl) pyrrolo[2,3-c]pyridin-4-yl]-4-nitro-phenoxy]phenoxy]ethoxy]ethoxy]acetate (378 mg, 515 μmol, 76% yield) as a white solid.

Step 4. A mixture of tert-butyl 2-[2-[2-[3-[2-[6-methyl-7-oxo-1-(p-tolylsulfonyl) pyrrolo[2,3-c]pyridin-4-yl]-4-nitro-phenoxy]phenoxy]ethoxy]ethoxy]acetate (510 mg, 695 μmol, 1.0 equiv), NH₄Cl (372 mg, 6.95 mmol, 10.0 equiv) in EtOH (5 mL) and H₂O (5 mL) was stirred at 80° C. for 12 h. The mixture was filtered and concentrated under reduced pressure to give a residue. The residue was purified by chromatography on a silica gel (petroleum ether: ethyl acetate=5/1 to 2/1). Compound tert-butyl 2-[2-[2-[3-[4-amino-2-[6-methyl-7-oxo-1-(p-tolylsulfonyl) pyrrolo[2,3-c]pyridin-4-yl]phenoxy]phenoxy]ethoxy]ethoxy]acetate (558 mg, 634 μmol, 91% yield, 80% purity) was obtained as a brown solid.

Step 5. To a solution of tert-butyl 2-[2-[2-[3-[4-amino-2-[6-methyl-7-oxo-1-(p-tolylsulfonyl) pyrrolo[2,3-c]pyridin-4-y]]phenoxy]phenoxy]ethoxy]ethoxy]acetate (640 mg, 909 μmol, 1.0 equiv) in DCM (5 mL) was added TEA (460 mg, 4.55 mmol, 633 μL, 5.0 equiv) and ethanesulfonyl chloride (585 mg, 4.55 mmol, 430 μL, 5.0 equiv) at 0° C. The resulting mixture was stirred at 23° C. for 12 h. The mixture filtered and concentrated under reduced pressure to give a residue. The mixture used to next step without purification. Compound tert-butyl 2-[2-[2-[3-[4-(ethylsulfonylamino)-2-[6-methyl-7-oxo-1-(p-tolylsulfonyl) pyrrolo[2,3-c]pyridin-4-yl]phenoxy]phenoxy]ethoxy]ethoxy]acetate (583 mg, 733 μmol, 81% yield) was obtained as a colorless oil.

Step 6. A mixture of tert-butyl 2-[2-[2-[3-[4-(ethylsulfonylamino)-2-[6-methyl-7-oxo-1-(p-tolylsulfonyl) pyrrolo[2,3-c]pyridin-4-y]]phenoxy]phenoxy]ethoxy]ethoxy]acetate (583 mg, 733 μmol, 1.0 equiv), NaOH (293 mg, 7.33 mmol, 10.0 equiv), in MeOH (6 mL) and H₂O (2 mL) was degassed and purged with N₂ for 3 times, and then the mixture was stirred at 50° C. for 2 h under N₂ atmosphere. The reaction mixture was adjusted to pH=4˜5 by HCl (1N) at 0° C., and then diluted with water (10 mL) and extracted with DCM (10 mL×3). The combined organic layers were washed with brine (10 mL), dried over with Na₂SO₄, filtered and concentrated under reduced pressure to give a residue. The residue was purified by prep-HPLC (FA condition, column: Phenomenex luna C18 150*25 mm*10 μm; mobile phase: [water (FA)-ACN]; B %: 30%-50%,58 min) to give the desired product. Compound 2-[2-[2-[3-[4-(ethylsulfonylamino)-2-(6-methyl-7-oxo-1H-pyrrolo[2,3-c]pyridin-4-yl)phenoxy]phenoxy]ethoxy]ethoxy]acetic acid (50 mg, 85 μmol, 12% yield) was obtained as a white solid.

Step 7. A mixture of 2-[2-[2-[3-[4-(ethylsulfonylamino)-2-(6-methyl-7-oxo-1H-pyrrolo[2,3-c]pyridin-4-yl)phenoxy]phenoxy]ethoxy]ethoxy]acetic acid (55 mg, 94 μmol, 1.0 equiv), (2S)—N-[(1S)-1-(2-chlorophenyl)-2-[(3,3-difluorocyclobutyl)amino]-2-oxo-ethyl]-1-(4-cyano-2-pyridyl)-N-(3,5-difluorophenyl)-6-oxo-piperazine-2-carboxamide (61 mg, 84 μmol, 0.9 equiv, TFA), HATU (54 mg, 141 μmol, 1.5 equiv), DIEA (61 mg, 469 μmol, 82 μL, 5.0 equiv) in DMF (3 mL) was degassed and purged with N₂ for 3 times, and then the mixture was stirred at 25° C. for 0.5 h under N₂ atmosphere. The mixture filtered and concentrated under reduced pressure to give a residue. The residue was purified by prep-HPLC (FA condition column: Phenomenex luna C18 150*25 mm*10 μm; mobile phase: [water (FA)-ACN]; B %: 51%-81%,10 min). Compound (2S)—N-[(1S)-1-(2-chlorophenyl)-2-[(3,3-difluorocyclobutyl)amino]-2-oxo-ethyl]-1-(4-cyano-2-pyridyl)-N-(3,5-difluorophenyl)-4-[2-[2-[2-[3-[4-(ethylsulfonylamino)-2-(6-methyl-7-oxo-1H-pyrrolo[2,3-c]pyridin-4-yl)phenoxy]phenoxy]ethoxy]ethoxy]acetyl]-6-oxo-piperazine-2-carboxamide (50.61 mg, 39.80 μmol, 42% yield, 93% purity) was obtained as a white solid. ¹H NMR: (400 MHZ, CD₃OD)8 =8.86-8.57 (m, 1H), 8.30-8.17 (m, 1H), 8.00-7.93 (m, 1H), 7.54-6.73 (m, 12H), 6.57-6.42 (m, 1H), 6.39-6.25 (m, 3H), 6.22-6.07 (m, 1H), 4.78-4.60 (m, 2H), 4.35-3.47 (m, 15H), 3.35-3.22 (m, 1H), 3.19-3.12 (m, 2H), 2.94-2.71 (m, 2H), 2.58-2.23 (m, 2H), 1.39-1.26 (m, 3H) LC-MS: MS (ES⁺): RT=3.077 min, m/z=1182.6 [M+H⁺]; LCMS method 100.

### Example—86 the Synthetic Route for I-108

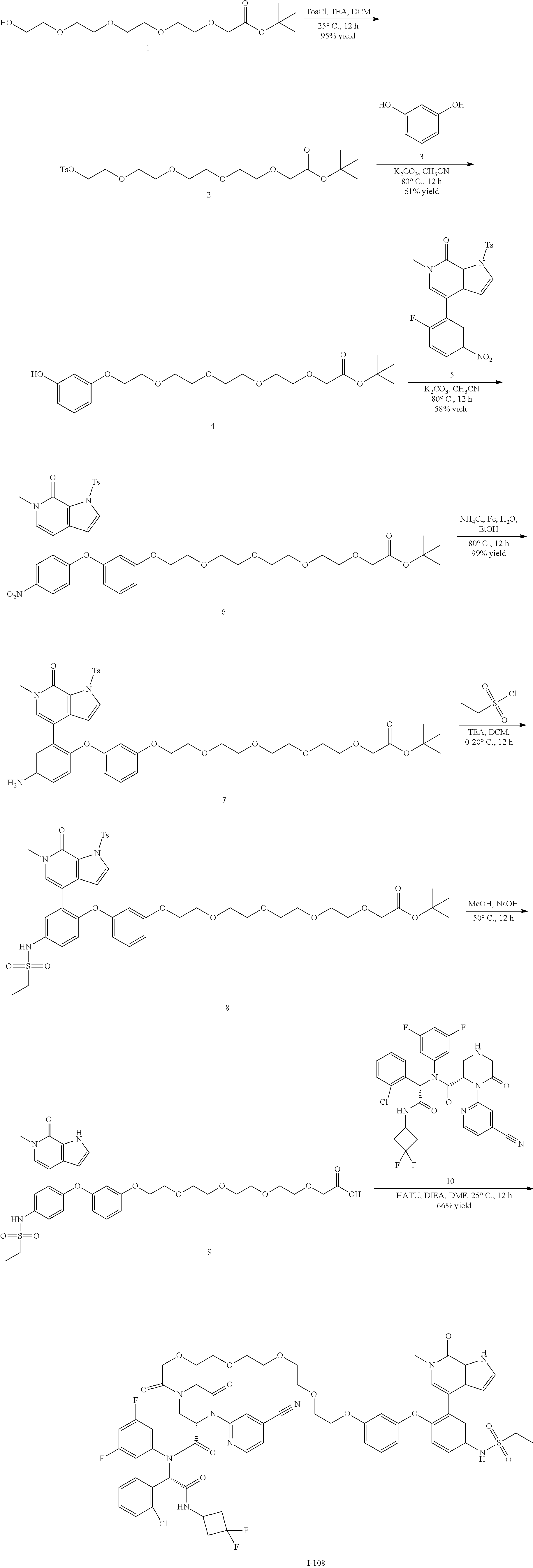

General Information: Synthetic route for compound 5 is described in Journal of Medicinal Chemistry, 2020, 63, 9093. Synthetic route for compound 10 is described in WO2013/107291, 2013, A¹.

Step 1. To a solution of tert-butyl 2-[2-[2-[2-(2-hydroxyethoxy)ethoxy]ethoxy]ethoxy]acetate (1.0 g, 3.2 mmol, 1.0 equiv), TEA (656 mg, 6.4 mmol, 902 μL, 2.0 equiv), TosCl (927 mg, 4.8 mmol, 1.5 equiv) in DCM (15 mL) was degassed and purged with N₂ for 3 times, and then the mixture was stirred at 25° C. for 12 h under N₂ atmosphere. The residue was diluted with H₂O (10 mL) and extracted with EA (3×10 mL). The combined organic layers were washed with brine (3×10 mL), dried over Na₂SO₄, filtered and concentrated under reduced pressure to give a residue. The residue was purified by column chromatography (SiO₂, Petroleum ether/Ethyl acetate=50/1 to 5/1) to give tert-butyl 2-[2-[2-[2-[2-(p-tolylsulfonyloxy)ethoxy]ethoxy]ethoxy]ethoxy]acetate (1.4 g, 3.0 mmol, 95% yield) as a yellow oil. ¹H NMR: (400 MHZ, CDCl₃) δ=7.79 (d, J=8.4 Hz, 2H), 7.34 (d, J=8.0 Hz, 2H), 4.17-4.12 (m, 2H), 4.03-3.98 (m, 2H), 3.70-3.66 (m, 6H), 3.64-3.60 (m, 4H), 3.57 (s, 4H), 2.44 (s, 3H), 1.46 (s, 9H)

Step 2. To a solution of tert-butyl 2-[2-[2-[2-[2-(p-tolylsulfonyloxy)ethoxy]ethoxy]ethoxy]ethoxy]acetate (1.3 g, 2.9 mmol, 1.0 equiv), benzene-1,3-diol (1.3 g, 11.9 mmol, 1.9 mL, 4.0 equiv), K₂CO₃ (1.6 g, 11.9 mmol, 4.0 equiv) in ACN(14 mL) was degassed and purged with N₂ for 3 times, and then the mixture was stirred at 80° C. for 12 h under N₂ atmosphere. The reaction mixture was filtered and concentrated under reduced pressure. The residue was purified by prep-HPLC (column: YMC Triart C18 250*50 mm*7 μm; mobile phase: [water (FA)-ACN]; B %: 25%-55%,23 min) to give tert-butyl 2-[2-[2-[2-[2-(3-hydroxyphenoxy)ethoxy]ethoxy]ethoxy]ethoxy]acetate (736 mg, 1.8 mmol, 61% yield) as a yellow oil. ¹H NMR: (400 MHZ, CDCl₃) δ=7.16-7.05 (m, 1H), 6.59-6.42 (m, 3H), 5.94-5.86 (m, 1H), 4.16-4.10 (m, 2H), 4.04 (s, 2H), 3.86-3.81 (m, 2H), 3.74-3.69 (m, 6H), 3.68-3.65 (m, 6H), 1.49-1.47 (m, 9H).

Step 3. To a solution of tert-butyl 2-[2-[2-[2-[2-(3-hydroxyphenoxy)ethoxy]ethoxy]ethoxy]ethoxy]acetate (285 mg, 711 μmol, 1.0 equiv), 4-(2-fluoro-5-nitro-phenyl)-6-methyl-1-(p-tolylsulfonyl) pyrrolo[2,3-c]pyridin-7-one (314 mg, 711 μmol, 1.0 equiv), K₂CO₃ (147 mg, 1.0 mmol, 1.5 equiv) in ACN(3 mL) was degassed and purged with N₂ for 3 times, and then the mixture was stirred at 80° C. for 12 h under N₂ atmosphere. The reaction mixture was filtered and concentrated under reduced pressure. The residue was purified by column chromatography (SiO₂, Petroleum ether/Ethyl acetate=50/1 to 5/1) to give tert-butyl 2-[2-[2-[2-[2-[3-[2-[6-methyl-7-oxo-1-(p-tolylsulfonyl) pyrrolo[2,3-c]pyridin-4-yl]-4-nitro-phenoxy]phenoxy]ethoxy]ethoxy]ethoxy]ethoxy]acetate (340 mg, 413 μmol, 58% yield) as a yellow solid.

Step 4. A solution of tert-butyl 2-[2-[2-[2-[2-[3-[2-[6-methyl-7-oxo-1-(p-tolylsulfonyl) pyrrolo[2,3-c]pyridin-4-yl]-4-nitro-phenoxy]phenoxy]ethoxy]ethoxy]ethoxy]ethoxy]acetate (510 mg, 620 μmol, 1.0 equiv), Fe (173 mg, 3.1 mmol, 5.0 equiv), NH₄Cl (331 mg, 6.2 mmol, 10.0 equiv) and H₂O (111 mg, 6.2 mmol, 111 μL, 10.0 equiv) in EtOH (5 mL) was degassed and purged with N₂ for 3 times, and then the mixture was stirred at 80° C. for 12 h under N₂ atmosphere. The reaction mixture was filtered and concentrated under reduced pressure. The residue was used for next step without further purification to give tert-butyl 2-[2-[2-[2-[2-[3-[4-amino-2-[6-methyl-7-oxo-1-(p-tolylsulfonyl) pyrrolo[2,3-c]pyridin-4-yl]phenoxy]phenoxy]ethoxy]ethoxy]ethoxy]ethoxy]acetate (491 mg, 620 μmol, 99% yield) as a yellow solid.

Step 5. To a solution of tert-butyl 2-[2-[2-[2-[2-[3-[4-amino-2-[6-methyl-7-oxo-1-(p-tolylsulfonyl) pyrrolo[2,3-c]pyridin-4-yl]phenoxy]phenoxy]ethoxy]ethoxy]ethoxy]ethoxy]acetate (462 mg, 583 μmol, 1.0 equiv), TEA (177 mg, 1.7 mmol, 243 μL, 3.0 equiv) in DCM (18 mL) was degassed and purged with N₂ for 3 times. Then the ethanesulfonyl chloride (750 mg, 5.8 mmol, 551 μL, 10.0 equiv) was added to the mixture and stirred at 0° C., and then the mixture was stirred at 20° C. for 12 h under N₂ atmosphere. The residue was diluted with H₂O (10 mL) and extracted with DCM (3×10 mL). The combined organic layers were washed with brine (3×10 mL), dried over Na₂SO₄, filtered and concentrated under reduced pressure to give a residue. The residue was used for next step without further purification to give tert-butyl 2-[2-[2-[2-[2-[3-[4-(ethylsulfonylamino)-2-[6-methyl-7-oxo-1-(p-tolylsulfonyl) pyrrolo[2,3-c]pyridin-4-yl]phenoxy]phenoxy]ethoxy]ethoxy]ethoxy]ethoxy]acetate (515 mg, crude) as a yellow oil.

Step 6. To a solution of tert-butyl 2-[2-[2-[2-[2-[3-[4-(ethylsulfonylamino)-2-[6-methyl-7-oxo-1-(p-tolylsulfonyl) pyrrolo[2,3-c]pyridin-4-y]]phenoxy]phenoxy]ethoxy]ethoxy]ethoxy]ethoxy]acetate (515 mg, 582 μmol, 1.0 equiv), NaOH (233 mg, 5.8 mmol, 10.0 equiv) in MeOH (5 mL) was degassed and purged with N₂ for 3 times, and then the mixture was stirred at 50° C. for 12 h under N₂ atmosphere. The reaction mixture was filtered and concentrated under reduced pressure. The residue was purified by prep-HPLC (column: Phenomenex luna C18 150*25 mm*10 μm; mobile phase: [water (FA)-ACN]; B %: 21%-51%, 10 min) to give 2-[2-[2-[2-[2-[3-[4-(ethylsulfonylamino)-2-(6-methyl-7-oxo-1H-pyrrolo[2,3-c]pyridin-4-yl)phenoxy]phenoxy]ethoxy]ethoxy]ethoxy]ethoxy]acetic acid (145 mg, 215 μmol, 36% yield) as a yellow solid. ¹H NMR: (400 MHz, CDCl₃) δ=11.34 (s, 1H), 7.49-7.42 (m, 1H), 7.39-7.32 (m, 2H), 7.18-7.10 (m, 1H), 7.06-6.97 (m, 2H), 6.54-6.33 (m, 3H), 6.27-6.19 (m, 1H), 4.21 (s, 2H), 3.94-3.88 (m, 2H), 3.78-3.68 (m, 7H), 3.65 (s, 8H), 3.60 (s, 3H), 3.25-3.16 (m, 2H), 1.48-1.40 (m, 3H).

Step 7. To a solution of 2-[2-[2-[2-[2-[3-[4-(ethylsulfonylamino)-2-(6-methyl-7-oxo-1H-pyrrolo[2,3-c]pyridin-4-yl)phenoxy]phenoxy]ethoxy]ethoxy]ethoxy]ethoxy]acetic acid (125 mg, 185 μmol, 1.0 equiv), (2S)—N-[1-(2-chlorophenyl)-2-[(3,3-difluorocyclobutyl)amino]-2-oxo-ethyl]-1-(4-cyano-2-pyridyl)-N-(3,5-difluorophenyl)-6-oxo-piperazine-2-carboxamide (128 mg, 176 μmol, 0.9 equiv, TFA), HATU (105 mg, 278 μmol, 1.5 equiv), DIEA (71 mg, 556 μmol, 96 μL, 3.0 equiv) in DMF (3 mL) was degassed and purged with N₂ for 3 times, and then the mixture was stirred at 25° C. for 12 h under N₂ atmosphere. The reaction mixture was filtered and concentrated under reduced pressure. The residue was purified by prep-HPLC (column: Phenomenex luna C18 150*25 mm*10 μm; mobile phase: [water (FA)-ACN]; B %: 48%-78%,10 min) to give (2S)—N-[1-(2-chlorophenyl)-2-[(3,3-difluorocyclobutyl)amino]-2-oxo-ethyl]-1-(4-cyano-2-pyridyl)-N-(3,5-difluorophenyl)-4-[2-[2-[2-[2-[2-[3-[4-(ethylsulfonylamino)-2-(6-methyl-7-oxo-1 H-pyrrolo[2,3-c]pyridin-4-yl)phenoxy]phenoxy]ethoxy]ethoxy]ethoxy]ethoxy]acetyl]-6-oxo-piperazine-2-carboxamide (158 mg, 124 μmol, 66% yield, 99% purity) as an off-white solid. ¹H NMR: (400 MHZ, MeOD) δ=8.78-8.59 (m, 1H), 8.39-8.22 (m, 1H), 8.01-7.73 (m, 1H), 7.55-7.38 (m, 3H), 7.35-7.16 (m, 4H), 7.14-6.70 (m, 6H), 6.55-6.23 (m, 5H), 5.21-4.93 (m, 1H), 4.79-4.51 (m, 2H), 4.46-4.01 (m, 4H), 3.96-3.83 (m, 2H), 3.79-3.49 (m, 17H), 3.30-3.07 (m, 3H), 3.00-2.74 (m, 2H), 2.58-2.30 (m, 2H), 1.40-1.30 (m, 3H). LC-MS: MS (ES⁺): RT=3.079 min, m/z=1270.6 [M+H⁺]; LCMS Method 100.

### Example 87—the Synthetic Route for I-109

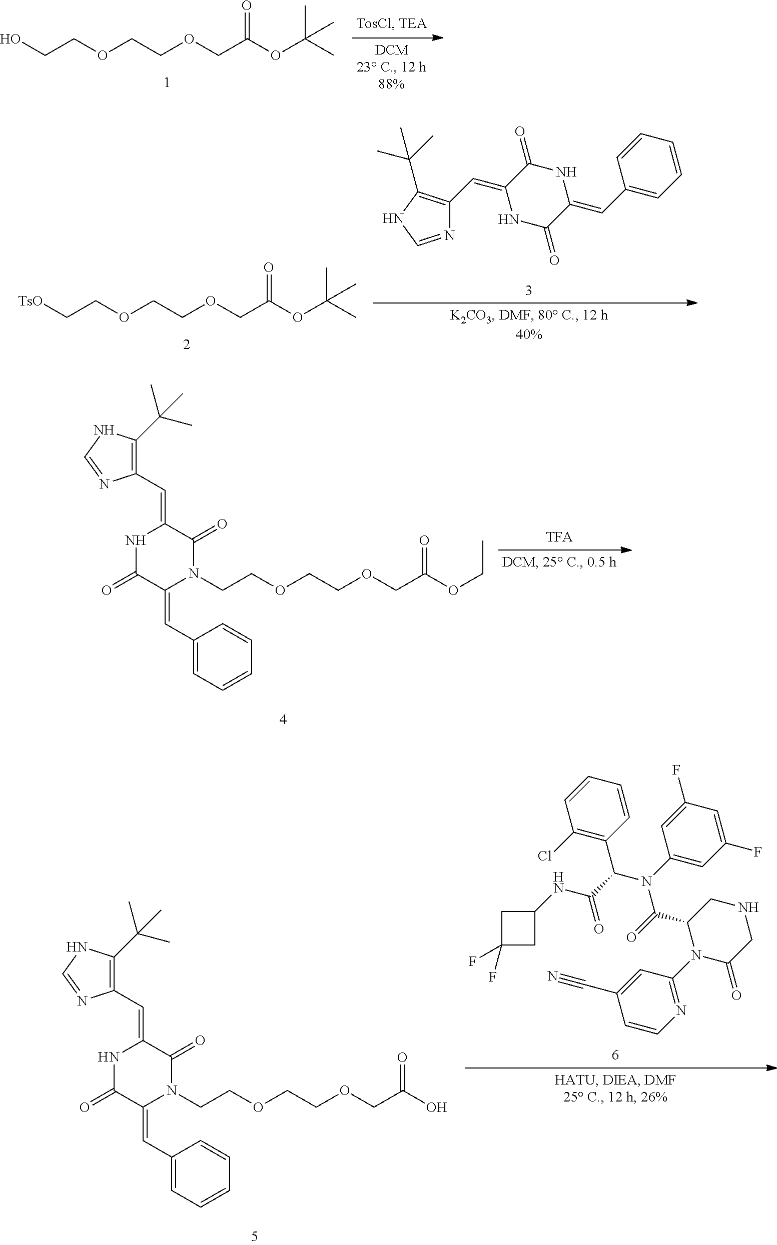 [SMILES: CC(C)(C)OC(=O)COCCOCCO ; CC(C)(C)c1[nH]cnc1/C=c1\[nH]c(=O)/c(=C/c2ccccc2)[nH]c1=O ; Cc1ccc(S(=O)(=O)OCCOCCOCC(=O)OC(C)(C)C)cc1 ; CCOC(=O)COCCOCCn1c(=O)/c(=C/c2nc[nH]c2C(C)(C)C)[nH]c(=O)/c1=C/c1ccccc1 ; CC(C)(C)c1[nH]cnc1/C=c1\[nH]c(=O)/c(=C/c2ccccc2)n(CCOCCOCC(=O)O)c1=O ; N#Cc1ccnc(N2C(=O)CNC[C@H]2C(=O)N(c2cc(F)cc(F)c2)[C@H](C(=O)NC2CC(F)(F)C2)c2ccccc2Cl)c1]

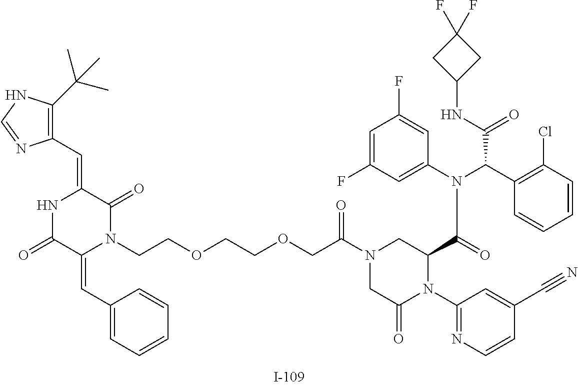 [SMILES: CC(C)(C)c1[nH]cnc1/C=c1\[nH]c(=O)/c(=C/c2ccccc2)n(CCOCCOCC(=O)N2CC(=O)N(c3cc(C#N)ccn3)[C@H](C(=O)N(c3cc(F)cc(F)c3)[C@H](C(=O)NC3CC(F)(F)C3)c3ccccc3Cl)C2)c1=O]

General Information: Compound 3 is described in WO2012/35436, 2012, A¹. Compound 6 is described in WO2013/107291, 2013, A¹.

Step 1. To a solution of tert-butyl 2-[2-(2-hydroxyethoxy)ethoxy]acetate (1.00 g, 4.54 mmol, 1.0 equiv) in DCM (10 mL) was added TEA (918 mg, 9.08 mmol, 2.0 equiv) and TosCl (1.30 g, 6.81 mmol, 1.5 equiv). The mixture was stirred at 25° C. for 12 h. The reaction mixture was concentrated to afford crude product. The residue was purified by silica chromatography (PE: EA=10:1-2:1) to afford tert-butyl 2-[2-[2-(p-tolylsulfonyloxy)ethoxy]ethoxy]acetate (1.50 g, 4.01 mmol, 88% yield) as a yellow oil.

Step 2. To a solution of tert-butyl 2-[2-[2-(p-tolylsulfonyloxy)ethoxy]ethoxy]acetate (400 mg, 1.07 mmol, 1.0 equiv) and (3Z,6Z)-3-benzylidene-6-[(5-tert-butyl-1H-imidazol-4-yl)methylene]piperazine-2,5-dione (359 mg, 1.07 mmol, 1.0 equiv) in DMF (10 mL) was added K₂CO₃ (443 mg, 3.20 mmol, 3.0 equiv). The mixture was stirred at 80° C. for 12 h. The reaction mixture was filtered to remove K₂CO₃ and concentrated to afford crude product. The residue was purified by prep-HPLC (column: Waters Xbridge C18 150*50 mm*10 μm; mobile phase: [water (NH₄HCO₃)-ACN]; B %: 48%-78%,10 min) to afford tert-butyl 2-[2-[2-[(2Z,5Z)-2-benzylidene-5-[(5-tert-butyl-1H-imidazol-4-yl)methylene]-3,6-dioxo-piperazin-1-yl]ethoxy]ethoxy]acetate (230 mg, 427 μmol, 40% yield) as a gray solid. ¹H NMR (400 MHZ, DMSO-d₆) δ 12.40-12.24 (m, 1H), 12.09 (s, 1H), 8.08 (d, J=7.5 Hz, 2H), 7.89-7.79 (m, 1H), 7.45-7.38 (m, 2H), 7.34-7.29 (m, 1H), 7.07 (s, 1H), 6.69-6.63 (m, 1H), 4.52-4.45 (m, 2H), 4.02-3.94 (m, 2H), 3.89-3.82 (m, 2H), 3.68-3.61 (m, 4H), 3.59 (s, 2H), 1.40-1.36 (m, 18H)

Step 3. To a solution of tert-butyl 2-[2-[2-[(2Z,5Z)-2-benzylidene-5-[(5-tert-butyl-1H-imidazol-4-yl)methylene]-3,6-dioxo-piperazin-1-yl]ethoxy]ethoxy]acetate (60.0 mg, 111 μmol, 1.0 equiv) in DCM (2 mL) was added TFA (1 mL), and then it was stirred at 25° C. for 0.5 h. The reaction mixture was concentrated to afford crude product. 2-[2-[2-[(2Z,5Z)-2-benzylidene-5-[(5-tert-butyl-1H-imidazol-4-yl)methylene]-3,6-dioxo-piperazin-1-yl]ethoxy]ethoxy]acetic acid (55.0 mg, crude) was used into the next step without further purification.

Step 4. To a solution of tert-butyl(3S)-3-[(1S)-1-(2-chlorophenyl)-2-[(3,3-difluorocyclobutyl)amino]-2-oxo-ethyl]-(3,5-difluorophenyl) carbamoyl]-4-(4-cyano-2-pyridyl)-5-oxo-piperazine-1-carboxylate (80.0 mg, 112 μmol, 1.0 equiv) in DCM (2 mL) was added TFA (1 mL). The mixture was stirred at 25° C. for 0.5 h. The reaction mixture was concentrated to afford crude product. (2S)—N-[(1S)-1-(2-chlorophenyl)-2-[(3,3-difluorocyclobutyl)amino]-2-oxo-ethyl]-1-(4-cyano-2-pyridyl)-N-(3,5-difluorophenyl)-6-oxo-piperazine-2-carboxamide (83 mg, TFA salt) was used into the next step without further purification.

Step 5. To a solution of 2-[2-[2-[(2Z,5Z)-2-benzylidene-5-[(5-tert-butyl-1H-imidazol-4-yl)methylene]-3,6-dioxo-piperazin-1-yl]ethoxy]ethoxy]acetic acid (50.0 mg, 104 μmol, 1.0 equiv) and (2S)—N-[(1S)-1-(2-chlorophenyl)-2-[(3,3-difluorocyclobutyl)amino]-2-oxo-ethyl]-1-(4-cyano-2-pyridyl)-N-(3,5-difluorophenyl)-6-oxo-piperazine-2-carboxamide (75.5 mg, 104 μmol, 1.0 equiv, TFA salt) in DMF (2 mL) was added DIEA (26.8 mg, 207 μmol, 2 equiv) and HATU (59.1 mg, 155 μmol, 1.5 equiv). The mixture was stirred at 25° C. for 0.5 h. The residue was purified by prep-HPLC (column: Phenomenex C18 75*30 mm*3 μm; mobile phase: [water (FA)-ACN]; B %: 65%-95%,7 min) to afford (2S)-4-[2-[2-[2-[(2Z,5Z)-2-benzylidene-5-[(5-tert-butyl-1H-imidazol-4-yl)methylene]-3,6-dioxo-piperazin-1-yl]ethoxy]ethoxy]acetyl]-N-[(1S)-1-(2-chlorophenyl)-2-[(3,3difluorocyclobutyl)amino]-2-oxo-ethyl]-1-(4-cyano-2-pyridyl)-N-(3,5-difluorophenyl)-6-oxo-piperazine-2-carboxamide (30.0 mg, 27.2 μmol, 26% yield, 98% purity) as a yellow solid. ¹H NMR (400 MHZ, DMSO-d₆) δ 8.31-7.66 (m, 6H), 7.53-7.15 (m, 6H), 6.98 (m, 2H), 6.93-6.83 (m, 1H), 6.71-6.58 (m, 1H), 6.34 (s, 1H), 5.02-4.75 (m, 1H), 4.39 (m, 6H), 4.12-3.83 (m, 4H), 3.83-3.42 (m, 6H), 1.22 (s, 9H). LC-MS: (ES⁺): RT=3.117 min, m/z=1079.3 [M+H⁺]; LCMS method 10.

### Example 88—the Synthetic Route for I-110

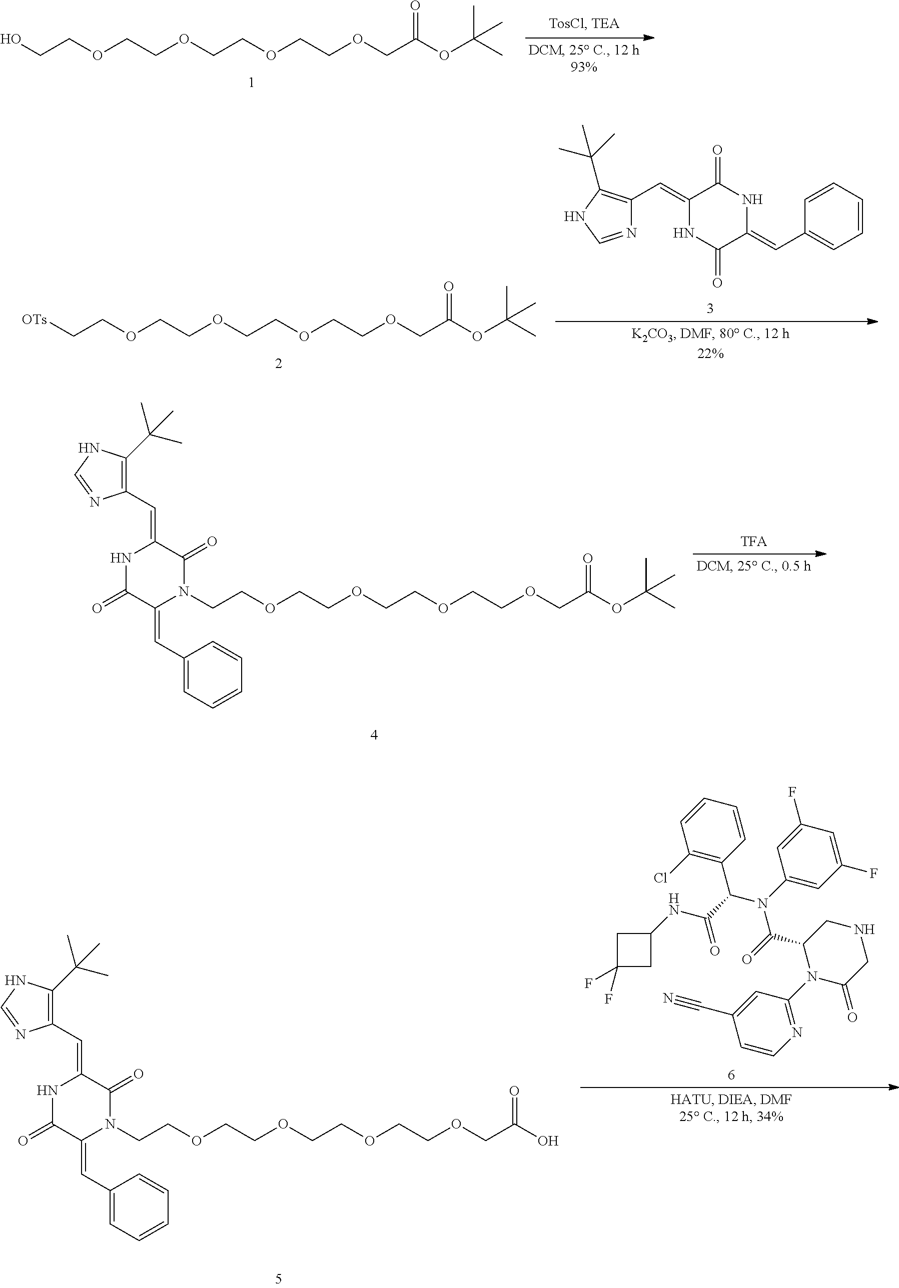 [SMILES: CC(C)(C)OC(=O)COCCOCCOCCOCCO ; CC(C)(C)c1[nH]cnc1/C=c1\[nH]c(=O)/c(=C/c2ccccc2)[nH]c1=O ; Cc1ccc(S(=O)(=O)OCCOCCOCCOCCOCC(=O)OC(C)(C)C)cc1 ; CC(C)(C)OC(=O)COCCOCCOCCOCCn1c(=O)/c(=C/c2nc[nH]c2C(C)(C)C)[nH]c(=O)/c1=C/c1ccccc1 ; CC(C)(C)c1[nH]cnc1/C=c1\[nH]c(=O)/c(=C/c2ccccc2)n(CCOCCOCCOCCOCC(=O)O)c1=O ; N#Cc1ccnc(N2C(=O)CNC[C@H]2C(=O)N(c2cc(F)cc(F)c2)[C@H](C(=O)NC2CC(F)(F)C2)c2ccccc2Cl)c1]

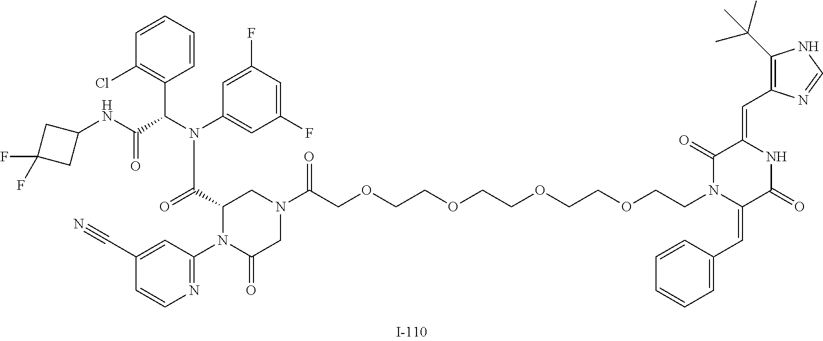 [SMILES: CC(C)(C)c1[nH]cnc1/C=c1\[nH]c(=O)/c(=C/c2ccccc2)n(CCOCCOCCOCCOCC(=O)N2CC(=O)N(c3cc(C#N)ccn3)[C@H](C(=O)N(c3cc(F)cc(F)c3)[C@H](C(=O)NC3CC(F)(F)C3)c3ccccc3Cl)C2)c1=O]

General Information: Compound 3 is described in WO2012/35436, 2012, A¹. Compound 6 is described in WO2013/107291, 2013, A¹.

Step 1. To a solution of tert-butyl 2-[2-[2-[2-(2-hydroxyethoxy)ethoxy]ethoxy]ethoxy]acetate (1.00 g, 3.24 mmol, 1.0 equiv) in DCM (10 mL) was added TEA (656 mg, 6.49 mmol, 2.0 equiv) and TosCl (927 mg, 4.86 mmol, 1.5 equiv). The mixture was stirred at 25° C. for 12 h. The reaction mixture was concentrated to afford crude product. The residue was purified by silica chromatography (PE:EA=10:1-1:1) to afford tert-butyl 2-[2-[2-[2-[2-(p-tolylsulfonyloxy)ethoxy]ethoxy]ethoxy]ethoxy]acetate (1.40 g, 3.03 mmol, 93% yield) as a yellow oil.

Step 2. To a solution of tert-butyl 2-[2-[2-[2-[2-(p-tolylsulfonyloxy)ethoxy]ethoxy]ethoxy]ethoxy]acetate (400 mg, 865 μmol, 1.0 equiv) and (3Z,6Z)-3-benzylidene-6-[(5-tert-butyl-1H-imidazol-4-yl)methylene]piperazine-2,5-dione (291 mg, 865 μmol, 1.0 equiv) in DMF (10 mL) was added K₂CO₃ (356 mg, 2.59 mmol, 3.0 equiv). The mixture was stirred at 80° C. for 12 h. The reaction mixture was filtered to remove K₂CO₃ and concentrated to afford crude product. The residue was purified by prep-HPLC (column: Waters Xbridge C18 150*50 mm*10 μm; mobile phase: [water (NH₄HCO₃)-ACN]; B %: 48%-78%,10 min) to afford tert-butyl 2-[2-[2-[2-[2-[(2Z,5Z)-2-benzylidene-5-[(5-tert-butyl-1 H-imidazol-4-yl)methylene]-3,6-dioxo-piperazin-1 yl]ethoxy]ethoxy]ethoxy]ethoxy]acetate (120 mg, 191 μmol, 22% yield), the product was a gray solid. ¹H NMR (400 MHZ, DMSO-d₆) δ 12.34-12.28 (m, 1H), 12.10-12.05 (m, 1H), 8.07 (d, J=7.3 Hz, 2H), 7.84-7.81 (m, 1H), 7.42-7.37 (m, 2H), 7.32-7.27 (m, 1H), 7.07-7.04 (m, 1H), 6.65-6.63 (m, 1H), 4.50-4.44 (m, 2H), 3.94-3.91 (m, 2H), 3.86-3.82 (m, 2H), 3.65-3.61 (m, 2H), 3.59-3.56 (m, 2H), 3.55-3.49 (m, 6H), 1.38-1.35 (m, 18H)

Step 3. To a solution of tert-butyl 2-[2-[2-[2-[2-[(2Z,5Z)-2-benzylidene-5-[(5-tert-butyl-1H-imidazol-4-yl)methylene]-3,6-dioxo-piperazin-1-yl]ethoxy]ethoxy]ethoxy]ethoxy]acetate (70.0 mg, 112 μmol, 1.0 equiv) in DCM (2 mL) was added TFA (1 mL). The mixture was stirred at 25° C. for 12 h. The reaction mixture was concentrated to afford crude 2-[2-[2-[2-[2-[(2Z,5Z)-2-benzylidene-5-[(5-tert-butyl-1H-imidazol-4-yl)methylene]-3,6-dioxo-piperazin-1-yl]ethoxy]ethoxy]ethoxy]ethoxy]acetic acid (65.0 mg, crude) was used into the next step without further purification.

Step 4. To a solution of 2-[2-[2-[2-[2-[(2Z,5Z)-2-benzylidene-5-[(5-tert-butyl-1H-imidazol-4-yl)methylene]-3,6-dioxo-piperazin-1-yl]ethoxy]ethoxy]ethoxy]ethoxy]acetic acid (50.0 mg, 87.6 μmol, 1.0 equiv) and (2S)—N-[(1S)-1-(2-chlorophenyl)-2-[(3,3-difluorocyclobutyl)amino]-2-oxo-ethyl]-1-(4-cyano-2-pyridyl)-N-(3,5-difluorophenyl)-6-oxo-piperazine-2-carboxamide (63.9 mg, 87.6 μmol, 1.0 equiv, TFA salt) in DMF (2 mL) was added DIEA (22.7 mg, 175 μmol, 2.0 equiv) and HATU (50.0 mg, 131 μmol, 1.5 equiv). The mixture was stirred at 25° C. for 0.5 h. The residue was purified by prep-HPLC (column: Phenomenex C18 75*30 mm*3 μm; mobile phase: [water (FA)-ACN]; B %: 60%-90%,7 min) to afford (2S)-4-[2-[2-[2-[2-[2-[(2Z,5Z)-2-benzylidene-5-[(5-tert-butyl-1 H-imidazol-4-yl)methylene]-3,6-dioxo-piperazin-1-yl]ethoxy]ethoxy]ethoxy]ethoxy]acetyl]-N-[(1S)-1-(2-chlorophenyl)-2-[(3,3-difluorocyclobutyl)amino]-2-oxo-ethyl]-1-(4-cyano-2-pyridyl)-N-(3,5-difluorophenyl)-6-oxo-piperazine-2-carboxamide (36.0 mg, 30.2 μmol, 34% yield, 98% purity) as a yellow solid. ¹H NMR (400 MHZ, DMSO-d₆) δ 9.04-8.62 (m, 2H), 8.35-7.99 (m, 3H), 7.96-7.67 (m, 3H), 7.51-7.36 (m, 3H), 7.34-7.05 (m, 5H), 6.98-6.85 (m, 1H), 6.70-6.56 (m, 1H), 6.41-6.07 (m, 1H), 5.07-4.81 (m, 1H), 4.67-4.37 (m, 4H), 4.37-3.94 (m, 4H), 3.93-3.60 (m, 6H), 3.59-3.38 (m, 10H), 2.96-2.80 (m, 2H), 2.45-2.23 (m, 2H), 1.38 (d, J=2.5 Hz, 8H). LC-MS: (ES⁺): RT=3.122 min, m/z=1167.3 [M+H⁺]; LCMS method 10.

### Example 82—the Synthetic Route for I-111

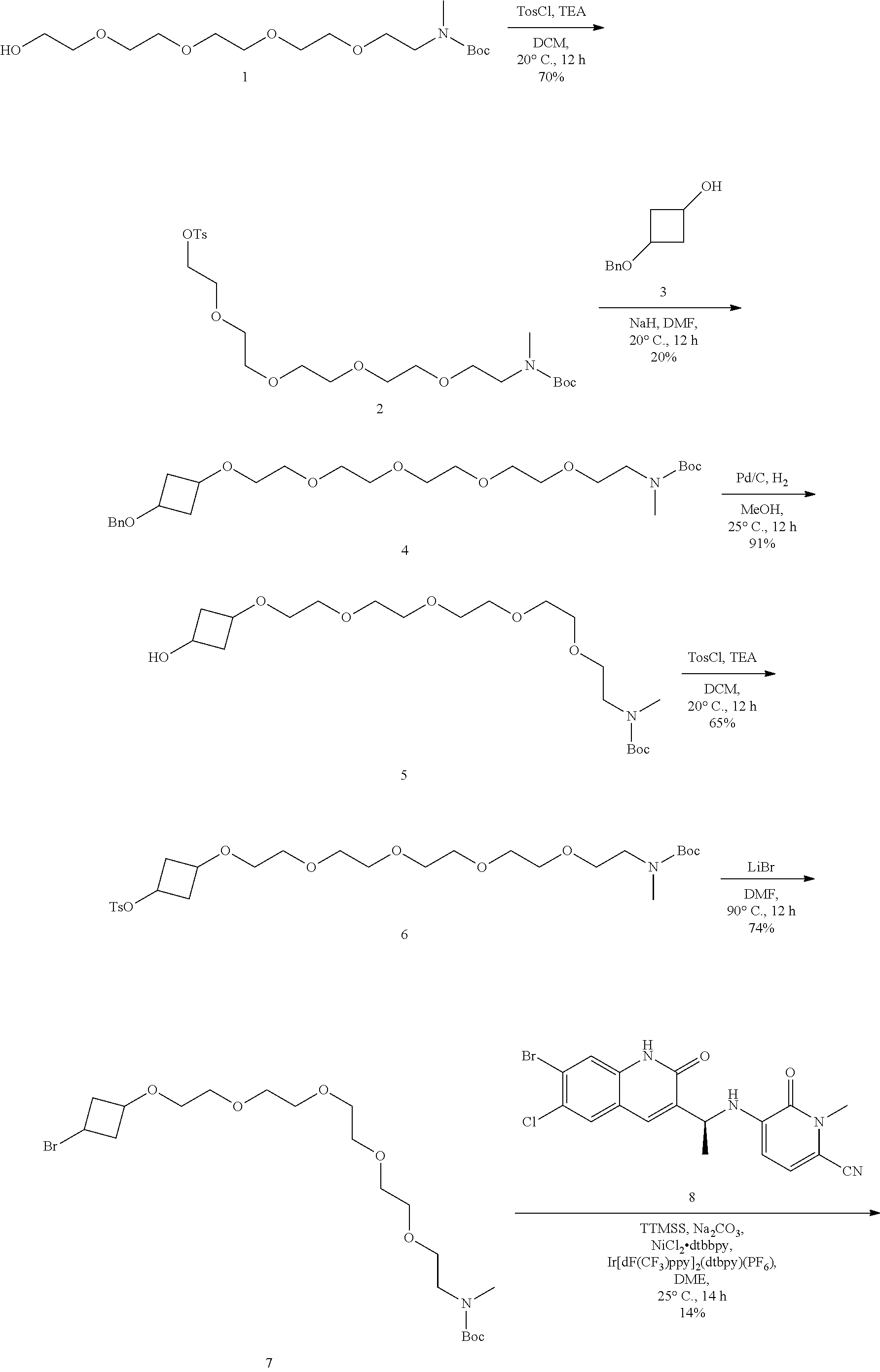

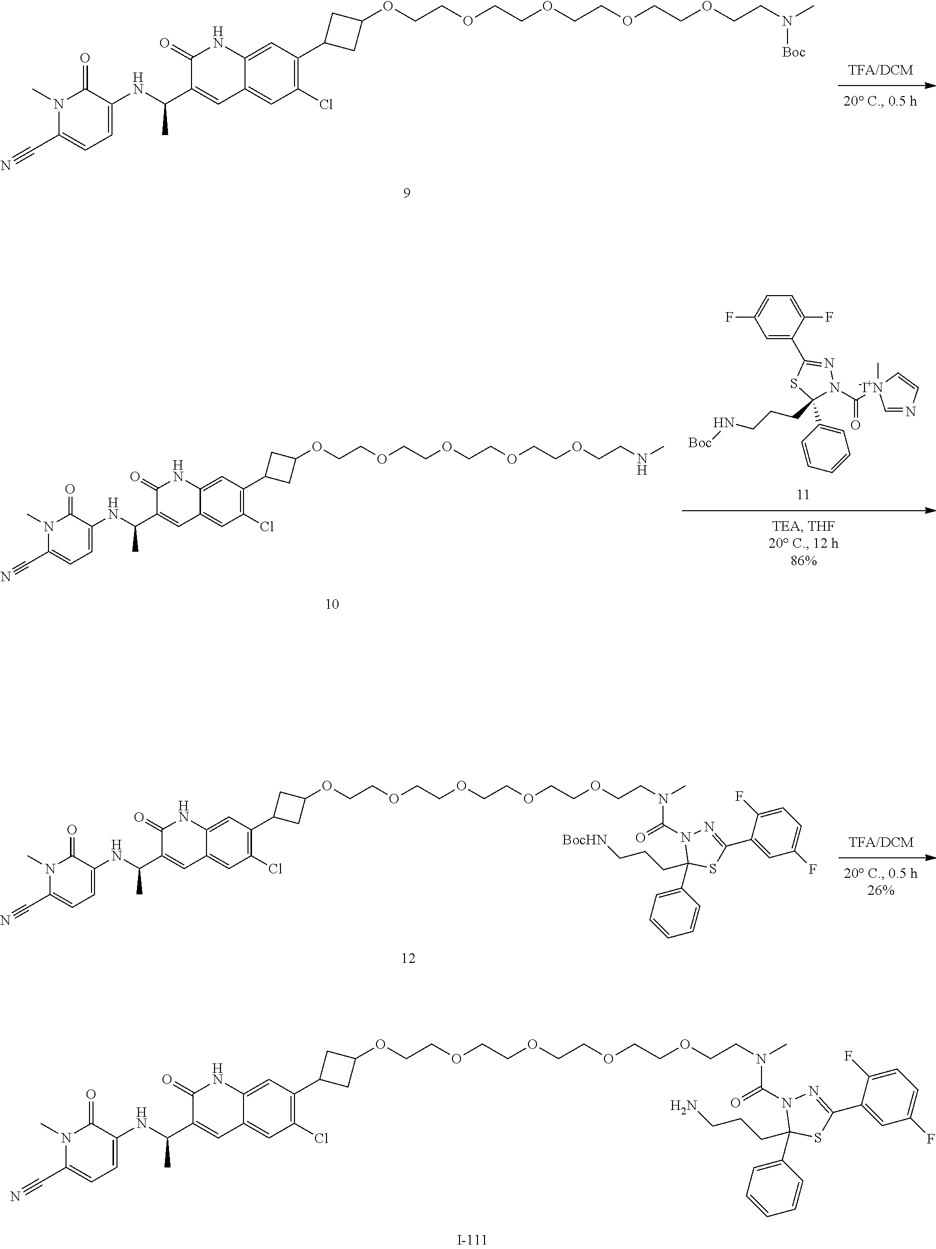

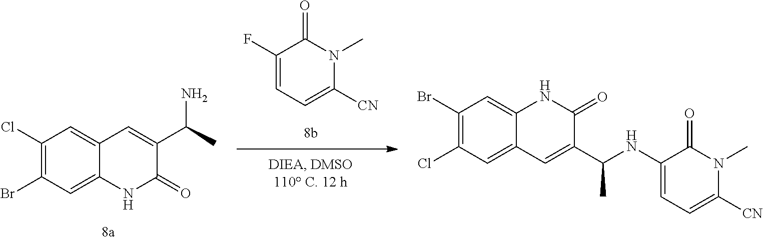 [SMILES: C[C@H](N)c1cc2cc(Cl)c(Br)cc2[nH]c1=O ; Cn1c(C#N)ccc(F)c1=O ; C[C@H](Nc1ccc(C#N)n(C)c1=O)c1cc2cc(Cl)c(Br)cc2[nH]c1=O]

General Information: Synthetic route for compound 1 is described in US2019/192668, 2019, A¹. Synthetic route for compound 8a is described in US2016/83365, 2016, A1. Synthetic route for compound 8b is described in US2019/350922, 2019, A¹. Synthetic route for compound 11 is described in US2006/247178 A¹.

Step 1. A mixture of tert-butyl N-[2-[2-[2-[2-(2-hydroxyethoxy)ethoxy]ethoxy]e thoxy]ethyl]-N-methyl-carbamate (2.0 g, 5.7 mmol, 1.0 equiv), TosCl (2.0 g, 11 mmol, 2.0 equiv), TEA (1.7 g, 17 mmol, 2.4 mL, 3.0 equiv) in DCM (20 mL) was degassed and purged with N₂ for 3 times, and then the mixture was stirred at 20° C. for 12 h under N₂ atmosphere. The reaction mixture was concentrated under reduced pressure to give a residue. The residue was purified by column chromatography (SiO₂, Petroleum ether/Ethyl acetate=1/1). Compound 2-[2-[2-[2-[2-[tert-butoxycarbonyl(methyl)amino]ethoxy]ethoxy]ethoxy]ethoxy]ethyl 4-methylbenzenesulfonate (2 g, 4 mmol, 70% yield) was obtained as a yellow oil.

Step 2. A mixture of 2-[2-[2-[2-[2-[tert-butoxycarbonyl(methyl)amino]ethoxy]ethoxy]ethoxy]ethoxy]ethyl 4-methylbenzenesulfonate (2 g, 4 mmol, 1.0 equiv), 3-benzyloxycyclobutanol (705 mg, 4 mmol, 1.0 equiv), NaH (474.7 mg, 12 mmol, 60% purity, 3.0 equiv) in DMF (20 mL) was degassed and purged with N₂ for 3 times, and then the mixture was stirred at 20° C. for 12 h under N₂ atmosphere. The residue was diluted with water (90 mL) and extracted with DCM (2×90 mL). The combined organic layers were washed with brine (90 mL), dried over Na₂SO₄, filtered and concentrated under reduced pressure to give a residue. The residue was purified by prep-HPLC (FA condition; column: Phenomenex luna C18 150*25 mm*10 μm; mobile phase: [water (FA)-ACN]; B %: 10%-40%,15 min). Compound tert-butyl N-[2-[2-[2-[2-[2-(3-benzyloxycyclobutoxy)ethoxy]ethoxy]ethoxy]ethoxy]ethyl]-N-methyl-carbamate (400 mg, 782 μmol, 20% yield) was obtained as a colorless oil. ¹H NMR: (400 MHZ, CDCl₃) δ=7.36-7.28 (m, 5H), 4.42 (s, 2H), 3.69-3.57 (m, 18H), 3.55-3.47 (m, 2H), 3.39 (br s, 2H), 2.91 (s, 3H), 2.70-2.57 (m, 2H), 2.05-1.93 (m, 2H), 1.61 (s, 3H), 1.46 (s, 9H).

Step 3. A mixture of tert-butyl N-[2-[2-[2-[2-[2-(3-benzyloxycyclobutoxy)ethoxy]ethoxy]ethoxy]ethoxy]ethyl]-N-methyl-carbamate (200 mg, 391 μmol, 1.0 equiv), Pd/C (200 mg, 10% purity), H2 (790 μg, 391 μmol, 1.0 equiv) in MeOH (10 mL) was degassed and purged with H₂ for 3 times, and then the mixture was stirred at 25° C. for 12 h under N₂ atmosphere. The mixture was filtered and the filtrate was concentrated to give a residue. The crude product was used into the next step without further purification. Compound tert-buty1N-[2-[2-[2-[2-[2-(3-hydroxycyclobutoxy)ethoxy]ethoxy]ethoxy]ethoxy]ethyl]-N-methyl-carbamate (150 mg, 356 μmol, 91% yield) was obtained as a colorless oil. ¹H NMR: (400 MHz, CDCl₃) δ=3.91 (br s, 1H), 3.72-3.61 (m, 16H), 3.51 (m, 2H), 3.40 (br s, 2H), 2.92 (s, 3H), 2.72 (m, 1H), 2.06-1.87 (m, 3H), 1.46 (s, 9H).

Step 4. A mixture of tert-butyl N-[2-[2-[2-[2-[2-(3-hydroxycyclobutoxy)ethoxy]ethoxy]ethoxy]ethoxy]ethyl]-N-methyl-carbamate (150 mg, 356 μmol, 1.0 equiv), TosCl (102 mg, 534 μmol, 1.5 equiv), TEA (72 mg, 712 μmol, 99 μL, 2.0 equiv) in DCM (3 mL) was degassed and purged with N₂ for 3 times, and then the mixture was stirred at 20° C. for 12 h under N₂ atmosphere. The mixture was filtered and the filtrate was concentrated to give a residue. The residue was purified by column chromatography (SiO₂, Petroleum ether/Ethyl acetate=1/1). Compound [3-[2-[2-[2-[2-[2-[tert-butoxycarbonyl(methyl)amino]ethoxy]ethoxy]ethoxy]ethoxy]ethoxy]cyclobutyl]4-methylbenzenesulfonate (130 mg, 226 μmol, 65% yield) was obtained as a yellow oil. ¹H NMR: (400 MHZ, CDCl₃) δ=7.79-7.77 (d, J=8.0 Hz, 2H), 7.35-7.33 (d, J=8.0 Hz, 2H), 4.46 (m, 1H), 4.13 (d, J=7.1 Hz, 1H), 3.65-3.51 (m, 17H), 3.48-3.36 (m, 4H), 2.91 (s, 3H), 2.62 (m, 2H), 2.45 (s, 3H), 2.22-2.07 (m, 2H), 1.46 (s, 9H).

Step 5. A mixture of [3-[2-[2-[2-[2-[2-[tert-butoxycarbonyl(methyl)amino]ethoxy]ethoxy]ethoxy]ethoxy]ethoxy]cyclobutyl]4-methylbenzenesulfonate (80 mg, 139 μmol, 1.0 equiv), LiBr (24 mg, 278 μmol, 7 μL, 2.0 equiv) in DMF (2 mL) was degassed and purged with N₂ for 3 times, and then the mixture was stirred at 90° C. for 12 h under N₂ atmosphere. The reaction mixture was filtered and the filtrate was concentrated under reduced pressure. The residue was diluted with water (40 mL) and extracted with EA (3×30 mL). The combined organic layers were washed with brine (3×30 mL), dried, filtered and concentrated under reduced pressure to give a residue. The residue was purified by column chromatography (SiO₂, Petroleum ether/Ethyl acetate=1/1). Compound tert-butyl N-[2-[2-[2-[2-[2-(3-bromo cyclobutoxy)ethoxy]ethoxy]ethoxy]ethoxy]ethyl]-N-methyl-carbamate (50 mg, 103 μmol, 74% yield) was obtained as a colorless oil.

Step 6. To a solution of 5-fluoro-1-methyl-6-oxo-pyridine-2-carbonitrile (292 mg, 1.92 mmol, 1.0 equiv) and 3-[(1S)-1-aminoethyl]-7-bromo-6-chloro-1H-quinolin-2-one (580 mg, 1.92 mmol, 1.0 equiv) in DMSO (6 mL) was added DIEA (497 mg, 3.85 mmol, 670 μL, 2.0 equiv). The mixture was stirred at 110° C. for 12 h. The reaction mixture was concentrated to give a residue. The residue was purified by prep-HPLC (FA) to afford 5-[[(1S)-1-(7-bromo-6-chloro-2-oxo-1H-quinolin-3-yl) ethyl]amino]-1-methyl-6-oxo-pyridine-2-carbonitrile (400 mg, 922 μmol, 48% yield) as a white solid. ¹H NMR: (400 MHz, DMSO-d₆) δ 7.95 (s, 1H), 7.74 (s, 1H), 7.64 (s, 1H), 6.96 (d, J=8.0 Hz, 1H), 6.89 (d, J=7.6 Hz, 1H), 5.94 (d, J=8.0 Hz, 1H), 4.67 (t, J=6.8 Hz, 1H), 3.58 (s, 3H), 1.50 (d, J=6.4 Hz, 3H).

Step 7. To a solution of tert-butyl N-[2-[2-[2-[2-[2-(3-bromocyclobutoxy)ethoxy]ethoxy]ethoxy]ethoxy]ethyl]-N-methyl-carbamate (110 mg, 227 μmol, 1.2 equiv) and 5-[(1S)-1-(7-bromo-6-chloro-2-oxo-1H-quinolin-3-yl) ethyl]amino]-1-methyl-6-oxo-pyridine-2-carbonitrile (82 mg, 189 μmol, 1.0 equiv) in DME (3 mL) was added TTMSS (47 mg, 189 μmol, 58 μL, 1.0 equiv), Na₂CO₃ (40 mg, 379 μmol, 2.0 equiv), NiCl₂·dtbbpy (1 mg, 3 μmol, 0.1 equiv), Ir[dF (CF₃) ppy]2 (dtbpy)(PF₆)(2 mg, 2 μmol, 0.01 equiv). The mixture was stirred at 25° C. under irradiation with a 34 W blue LED lamp (7 cm away) for 14 h. The reaction mixture was concentrated under reduced pressure to give a residue. The residue was purified by column chromatography (SiO₂, Petroleum ether/Ethyl acetate=0/1). Compound tert-butyl N-[2-[2-[2-[2-[2-[3-[6-chloro-3-[(IR)-1-[(6-cyano-1-methyl-2-oxo-3-pyridyl)amino]ethyl]-2-oxo-1H-quinolin-7-yl]cyclobutoxy]ethoxy]ethoxy]ethoxy]ethoxy]ethyl]-N-methyl-carbamate (20 mg, 26 μmol, 14% yield) was obtained as a white solid.

Step 8. To a mixture of tert-butyl N-[2-[2-[2-[2-[2-[3-[6-chloro-3-[(1R)-1-[(6-cyano-1-methyl-2-oxo-3-pyridyl)amino]ethyl]-2-oxo-1H-quinolin-7-yl]cyclobutoxy]ethoxy]ethoxy]ethoxy]ethoxy]ethyl]-N-methyl-carbamate (20 mg, 26.37 μmol, 1.0 equiv) in DCM (1 mL) was added TFA (0.5 mL). The mixture was stirred at 20° C. for 0.5 h. The mixture was concentrated to give a residue. The residue 5-[(1R)-1-[6-chloro-7-[3-[2-[2-[2-[2-[2-(methylamino)ethoxy]ethoxy ethoxy]ethoxy]ethoxy]cyclobutyl]-2-oxo-1H-quinolin-3-y]ethyl]amino]-1-methyl-6-oxo-pyridine-2-carbonitrile (20 mg, 26 μmol, crude, TFA) as a yellow oil was used for next step directly.

Step 9. To a mixture of 5-[[(IR)-1-[6-chloro-7-[3-[2-[2-[2-[2-[2-(methylamino) ethoxy]ethoxy]ethoxy]ethoxy]ethoxy]cyclobutyl]-2-oxo-1H-quinolin-3-yl]ethyl]amino]-1-methyl-6-oxo-pyridine-2-carbonitrile (20.0 mg, 25.9 μmol, 1.0 equiv, TFA) in DCM (1 mL) was added tert-butyl N-[3-[5-(2,5-difluorophenyl)-3-(1-methylimidazol-1-ium-1-carbonyl)-2-phenyl-1,3,4-thiadiazol-2-yl]propyl]carbamate; iodide (20.8 mg, 31.1 μmol, 1.2 equiv) and DIEA (10.0 mg, 77.7 μmol, 13.5 μL, 3.0 equiv). The mixture was stirred at 20° C. for 12 h. The mixture was concentrated to give a residue. The residue was purified by prep-TLC (Dichloromethane: Methanol=10:1) to give the desired product tert-butyl N-[3-[3-[2-[2-[2-[2-[2-[3-[6-chloro-3-[(1R)-1-[(6-cyano-1-methyl-2-oxo-3-pyridyl)amino]ethyl]-2-oxo-1H-quinolin-7-yl]cyclobutoxy]ethoxy]ethoxy]ethoxy]ethoxy]ethyl-methyl-carbamoyl]-5-(2,5-difluorophenyl)-2-phenyl-1,3,4-thiadiazol-2-yl]propyl]carbamate (25 mg, 22.4 μmol, 86% yield) as a yellow oil

Step 10. To a mixture of tert-butyl N-[3-[3-[2-[2-[2-[2-[2-[3-[6-chloro-3-[(1R)-1-[(6-cyano-1-methyl-2-oxo-3-pyridyl)amino]ethyl]-2-oxo-1H-quinolin-7-yl]cyclobutoxy]ethoxy]ethoxy]ethoxy]ethoxy]ethyl-methyl-carbamoyl]-5-(2,5-difluorophenyl)-2-phenyl-1,3,4-thiadiazol-2-yl]propyl]carbamate (25 mg, 22.4 μmol, 1 equiv) in TFA (0.5 mL) was added DCM (1 mL). The mixture was stirred at 20° C. for 0.5 h. The mixture was concentrated to give a residue. The residue was purified by prep-HPLC (FA) to give the desired product 2-(3-aminopropyl)-N-[2-[2-[2-[2-[2-[3-[6-chloro-3-[(1R)-1-[(6-cyano-1-methyl-2-oxo-3-pyridyl)amino]ethyl]-2-oxo-1H-quinolin-7-yl]cyclobutoxy]ethoxy]ethoxy]ethoxy]ethoxy]ethyl]-5-(2,5-difluorophenyl)-N-methyl-2-phenyl-1,3,4-thiadiazole-3-carboxamide (6.56 mg, 5.92 μmol, 26% yield, 96.0% purity, FA) as a green solid ¹H NMR: (400 MHZ, CD₃OD) δ=8.54 (s, 1H), 7.71 (m, 1H), 7.55-7.47 (m, 4H), 7.35-7.23 (m, 6H), 6.82-6.72 (m, 1H), 6.04-5.91 (m, 1H), 4.76 (m, 2H), 4.15 (m, 1H), 3.69-3.61 (m, 24H), 3.32 (m, 2H), 3.13-3.06 (m, 6H), 2.84 (m, 2H), 2.50 (m, 2H), 2.00 (m, 4H), 1.60 (m, 3H). LC-MS: MS (ES⁺): RT=2.727 min, m/z=1017.6 [M+H⁺]; LCMS method 05.

### Example 90—the Synthetic Route for I-112

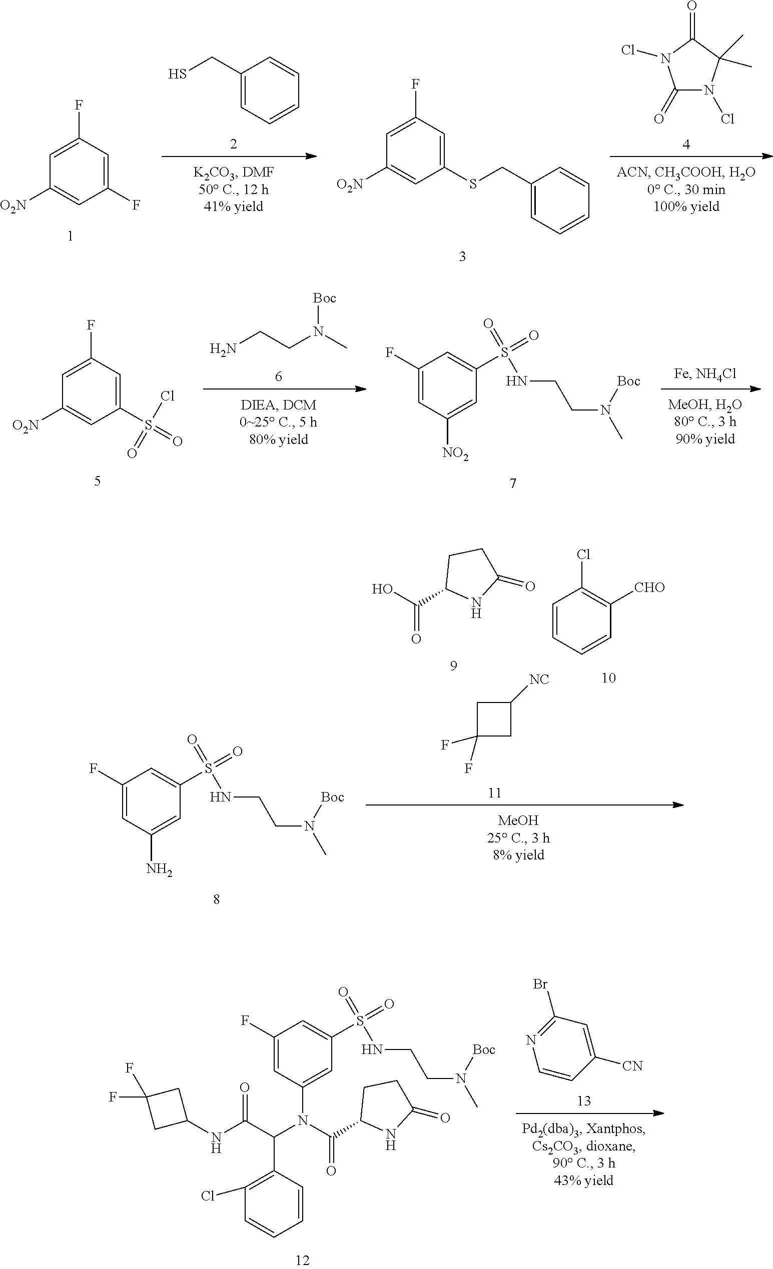

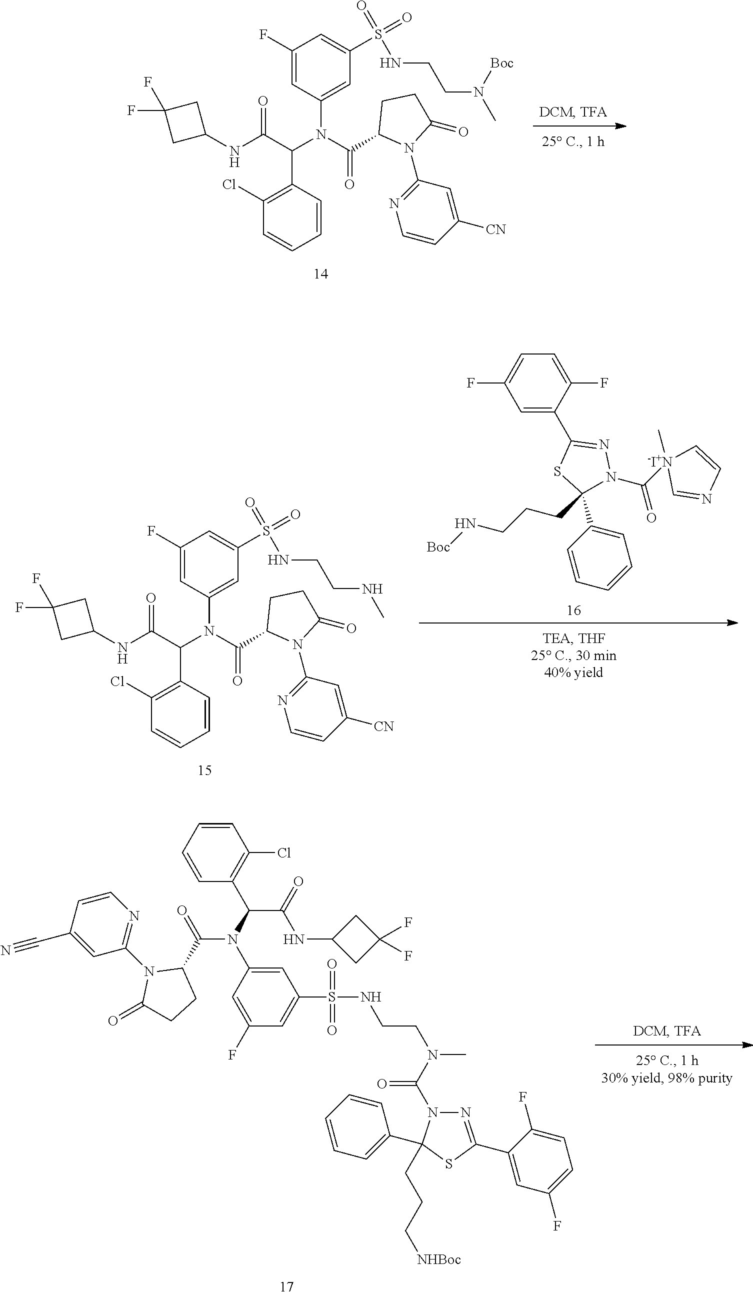

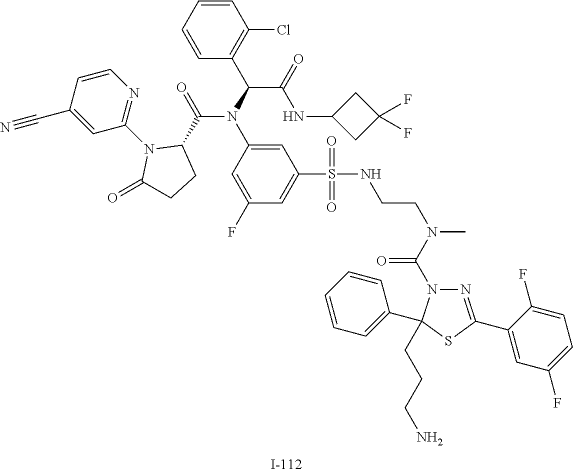 [SMILES: CN(CCNS(=O)(=O)c1cc(F)cc(N(C(=O)[C@@H]2CCC(=O)N2c2cc(C#N)ccn2)[C@H](C(=O)NC2CC(F)(F)C2)c2ccccc2Cl)c1)C(=O)N1N=C(c2cc(F)ccc2F)SC1(CCCN)c1ccccc1]

General Information: Synthetic route for compound 11 is described in WO2013/107405. Synthetic route for compound 16 is described in US2006/247178 A1.

Step 1. To a solution of 1,3-difluoro-5-nitro-benzene (10.0 g, 62.8 mmol, 1.0 equiv) in DMF (100 mL) was added K₂CO₃ (26.1 g, 189 mmol, 3.0 equiv) andphenylmethanethiol (8.6 g, 69 mmol, 8.1 mL, 1.1 equiv). The mixture was stirred at 50° C. for 12 h. The resultant mixture was quenched with saturated aqueous solution of H₂O (500 mL). The aqueous phase was extracted with ethyl acetate (2×500 mL). The combined organic phase was washed with brine (2×500 mL), dried over anhydrous sodium sulfate, filtered and concentrated under vacuum to give a residue. The residue was purified by flash silica gel chromatography (ISCO®; 80 g SepaFlash® Silica Flash Column, Eluent of 0 ˜10% Ethyl acetate/Petroleum ethergradient @ 60 mL/min) to afford 1-benzylsulfanyl-3-fluoro-5-nitro-benzene (6.8 g, 26 mmol, 41% yield) as a yellow solid. ¹H NMR: (400 MHZ, DMSO-d₆) δ=7.98-7.94 (m, 1H), 7.89-7.83 (m, 1H), 7.77-7.72 (m, 1H), 7.45-7.41 (m, 2H), 7.37-7.31 (m, 2H), 7.27 (d, J=7.2 Hz, 1H), 4.45 (s, 2H).

Step 2. To a 100 mL round-bottomed flask was added 1-benzylsulfanyl-3-fluoro-5-nitro-benzene (3.2 g, 12 mmol, 1.0 equiv), ACN(39 mL), H₂O (1 mL) and CH₃COOH (1.6 mL). The reaction mixture was cooled to 0° C. and 1,3-dichloro-5,5-dimethyl-imidazolidine-2,4-dione (3.3 g, 17 mmol, 1.4 equiv) was added portion-wised. When the addition was finished, the reaction mixture was stirred at 0° C. for 30 min. The reaction mixture was then diluted with salt NaHCO₃70 ml and extracted with EtOAc (140 mL). The organic extract was washed with water and dried over Na₂SO₄ and used for next step directly. Compound 3-fluoro-5-nitro-benzenesulfonyl chloride (2.9 g, 12 mmol, 100% yield) as a yellow solid.

Step 3. To a solution of tert-butyl N-(2-aminoethyl)-N-methyl-carbamate (330 mg, crude) in DCM (5 mL) was added DIEA (368 mg, 2.8 mmol, 496 μL, 1.5 equiv) and 3-fluoro-5-nitro-benzenesulfonyl chloride (455 mg, 1.90 mmol, 1.0 equiv) in 0° C. Then, the mixture was stirred at 25° C. for 5 h. The reaction mixture was partitioned between DCM (20 mL) and H₂O (10 mL). The organic phase was separated, washed with aqueous NaCl 10 mL (2×5 mL), dried over Na₂SO₄, filtered and concentrated under reduced pressure to give a residue. The residue was purified by flash silica gel chromatography (ISCO®; 12 g SepaFlash® Silica Flash Column, Eluent of 0˜30% Ethyl acetate/Petroleum ethergradient at 55 mL/min) to afford tert-butyl N-[2-[(3-fluoro-5-nitro-phenyl)sulfonylamino]ethyl]-N-methyl-carbamate (580 mg, 1.5 mmol, 80% yield) as a yellow oil. ¹H NMR: (400 MHZ, DMSO-d₆) δ=8.49-8.41 (m, 1H), 8.40-8.33 (m, 1H), 8.25-8.16 (m, 1H), 8.14-8.03 (m, 1H), 3.24-3.15 (m, 2H), 2.96 (s, 2H), 2.72 (s, 2H), 1.36 (s, 9H).

Step 4. To a solution of tert-butyl N-[2-[(3-fluoro-5-nitro-phenyl)sulfonylamino]ethyl]-N-methyl-carbamate (700 mg, 1.9 mmol, 1.0 equiv) in MeOH (7 mL) was added Fe (518 mg, 9.3 mmol, 5.0 equiv) and NH₄Cl (992 mg, 18.5 mmol, 10 equiv) in H₂O (7 mL). The mixture was stirred at 80° C. for 3 h. The reaction mixture was filtered. The residue was diluted with Ethyl acetate (40 mL). The organic layers were washed with aqueous NaCl (2×10 mL), dried over Na₂SO₄, filtered and concentrated under reduced pressure to give a residue. The residue was purified by flash silica gel chromatography (ISCO®; 12 g SepaFlash® Silica Flash Column, Eluent of 0˜10% Ethyl acetate/Petroleum ethergradient at 55 mL/min) to afford tert-butyl N-[2-[(3-amino-5-fluoro-phenyl)sulfonylamino]ethyl]-N-methyl-carbamate (580 mg, 1.7 mmol, 90% yield) as a yellow oil. ¹H NMR: (400 MHZ, DMSO-d₆)8 =7.68-7.57 (m, 1H), 6.82 (s, 1H), 6.65-6.45 (m, 2H), 5.93 (s, 2H), 3.22-3.13 (m, 2H), 2.85 (d, J=6.4 Hz, 2H), 2.75 (s, 3H), 1.37 (s, 9H).

Step 5. To a mixture of (2S)-5-oxopyrrolidine-2-carboxylic acid (189 mg, 1.47 mmol, 1.0 equiv)2-chlorobenzaldehyde (206 mg, 1.5 mmol, 165 μL, 1.0 equiv) in MeOH (2 mL) was stirred at 25° C. for 0.5 h. Then tert-butyl N-[2-[(3-amino-5-fluoro-phenyl)sulfonylamino]ethyl]-N-methyl-carbamate (510 mg, 1.5 mmol, 1.0 equiv) was added, followed by 3,3-difluorocyclo butanecarbonitrile (516 mg, 4.4 mmol, 3.0 equiv). The mixture was stirred at 25° C. for 2.5 h. The residue was purified by prep-HPLC (column: Phenomenex luna C18 150*25 mm*10 μm; mobile phase: [water (FA)-ACN]; B %: 34%-64%,10 min) to afford tert-butyl N-[2-[[3-[[1-(2-chlorophenyl)-2-[(3,3-difluorocyclobutyl)amino]-2-oxo-ethyl]-[(2S)-5-oxopyrrolidine-2-carbonyl]amino]-5-fluoro-phenyl]sulfonylamino Jethyl]-N-methyl-carbamate (90 mg, 125 μmol, 8% yield) as a white solid. ¹H NMR: (400 MHZ, DMSO-d₆)8=9.05-8.94 (m, 1H), 8.12-7.97 (m, 1H), 7.92-7.74 (m, 1H), 7.47 (d, J=7.6 Hz, 2H), 7.28-7.15 (m, 1H), 7.14-7.00 (m, 1H), 6.98-6.82 (m, 1H), 6.33 (s, 1H), 5.76 (s, 1H), 4.24-3.84 (m, 2H), 3.37 (s, 2H), 3.24-3.12 (m, 2H), 3.05-2.87 (m, 2H), 2.77 (d, J=8.8 Hz, 3H), 2.48-2.32 (m, 1H), 2.23-1.70 (m, 4H), 1.40 (s, 9H).

Step 6. A mixture of 2-bromopyridine-4-carbonitrile (15 mg, 83 μmol, 1.2 equiv), tert-butyl N-[2-[[3-[[1-(2-chlorophenyl)-2-[(3,3-difluorocyclobutyl)amino]-2-oxo-ethyl]-[(2S)-5-oxopyrrolidine-2-carbonyl]amino]-5-fluoro-phenyl]sulfonylamino]ethyl]-N-methyl-carbamate (50 mg, 69 μmol, 1.0 equiv), Xantphos (8 mg, 14 μmol, 0.2 equiv), Cs₂CO₃ (68 mg, 209 μmol, 3.0 equiv) and Pd₂ (dba)3 (6 mg, 7 μmol, 0.1 equiv) in dioxane (2 mL) was degassed and purged with N₂ for 3 time and then the mixture was stirred at 90° C. for 3 h under N₂ atmosphere. The residue was purified by prep-TLC (SiO2, DCM: MeOH=10:1) to afford tert-butyl N-[2-[[3-[[1-(2-chlorophenyl)-2-[(3,3-difluorocyclobutyl)amino]-2-oxo-ethyl]-[(2S)-1-(4-cyano-2-pyridyl)-5-oxo-pyrrolidine-2-carbonyl]amino]-5-fluoro-phenyl]sulfonylamino]ethyl]-N-methyl-carbamate (25 mg, 30 μmol, 43% yield) as a yellow oil.

Step 7. To a solution of tert-butyl N-[2-[[3-[1-(2-chlorophenyl)-2-[(3,3-difluorocyclobutyl)amino]-2-oxo-ethyl]-[(2S)-1-(4-cyano-2-pyridyl)-5-oxo-pyrrolidine-2-carbonyl]amino]-5-fluoro-phenyl]sulfonylamino]ethyl]-N-methyl-carbamate (55 mg, 67 μmol, 1.0 equiv) in DCM (2 mL) was added TFA (38 mg, 336 μmol, 25 μL, 5.0 equiv). The mixture was stirred at 25° C. for 1 h. The reaction mixture was filtered and concentrated under vacuum to give a residue. The crude product (2S)—N-[1-(2-chlorophenyl)-2-[(3,3-difluorocyclobutyl)amino]-2-oxo-ethyl]-1-(4-cyano-2-pyridyl)-N-[3-fluoro-5-[2-(methylamino) ethylsulfamoyl]phenyl]-5-oxo-pyrrolidine-2-carboxamide (48.2 mg, crude, TFA) was yellow oil and used into the next step without further purification.

Step 8. A mixture of (2S)—N-[1-(2-chlorophenyl)-2-[(3,3-difluorocyclobutyl)amino]-2-oxo-ethyl]-1-(4-cyano-2-pyridyl)-N-[3-fluoro-5-[2-(methylamino) ethylsulfamoyl]phenyl]-5-oxo-pyrrolidine-2-carboxamide (45 mg, 63 μmol, 1.0 equiv), tert-butyl N-[3-[(2S)-5-(2,5-difluorophenyl)-3-(1-iodoimidazol-1-ium-1-carbonyl)-2-phenyl-1,3,4-thiadiazol-2-yl]propyl]carbamate (49 mg, 75 μmol, 1.2 equiv), TEA (19 mg, 188 μmol, 26 μL, 3.0 equiv) in THF (0.5 mL) was degassed and purged with N₂ for 3 times, and then the mixture was stirred at 25° C. for 30 min under N₂ atmosphere. The reaction mixture was filtered and concentrated under reduced pressure to give a residue. The residue was purified by prep-HPLC (column: Welch Ultimate C18 150*25 mm*5 μm; mobile phase: [water (FA)-ACN]; B %: 68%-98%, min) to give the tert-butyl N-[3-[3-[2-[[3-[(1S)-1-(2-chlorophenyl)-2-[(3,3-difluorocyclobutyl)amino]-2-oxo-ethyl]-[(2S)-1-(4-cyano-2-pyridyl)-5-oxo-pyrrolidine-2-carbonyl]amino]-5-fluoro-phenyl]sulfonylamino]ethyl-methyl-carbamoyl]-5-(2,5-difluorophenyl)-2-phenyl-1,3,4-thiadiazol-2-yl]propyl]carbamate (30 mg, 25 μmol, 40% yield) as a white solid.

Step 9. A mixture of tert-butyl N-[3-[3-[2-[[3-[[(1S)-1-(2-chlorophenyl)-2-[(3,3-difluorocyclobutyl)amino]-2-oxo-ethyl]-[(2S)-1-(4-cyano-2-pyridyl)-5-oxo-pyrrolidine-2-carbonyl]amino]-5-fluoro-phenyl]sulfonylamino]ethyl-methyl-carbamoyl]-5-(2,5-difluorophenyl)-2-phenyl-1,3,4-thiadiazol-2-yl]propyl]carbamate (30 mg, 25 μmol, 1.0 equiv) in DCM (1 mL), TFA (0.3 mL) was degassed and purged with N₂ for 3 times, and then the mixture was stirred at 25° C. for 1 h under N₂ atmosphere. The reaction mixture was filtered and concentrated under reduced pressure to give a residue. The residue was purified by prep-HPLC (column: Waters xbridge 150*25 mm*10 μm; mobile phase: [water (FA)-ACN]; B %: 28%-58%,8 min). Compound 2-(3-aminopropyl)-N-[2-[[3-[[(1S)-1-(2-chlorophenyl)-2-[(3,3-difluorocyclobutyl)amino]-2-oxo-ethyl]-[(2S)-1-(4-cyano-2-pyridyl)-5-oxo-pyrrolidine-2-carbonyl]amino]-5-fluoro-phenyl]sulfonylamino]ethyl]-5-(2,5-difluorophenyl)-N-methyl-2-phenyl-1,3,4-thiadiazole-3-carboxamide (9 mg, 8 μmol, 30% yield, 98% purity, FA) as a white solid. ¹H NMR: (400 MHZ, DMSO-d₆) δ=9.04-8.79 (m, 1H), 8.71-8.45 (m, 2H), 8.44-8.15 (m, 3H), 7.97-7.70 (m, 1H), 7.66-6.85 (m, 13H), 6.39-6.20 (m, 1H), 4.90-4.57 (m, 2H), 4.21-3.98 (m, 3H), 3.32-3.16 (m, 8H), 3.03-2.76 (m, 10H), 2.64 (s, 1H), 2.44-2.31 (m, 1H), 2.09-1.84 (m, 2H), 1.66-1.34 (m, 1H). LC-MS: MS (ES⁺): RT=2.699 min, m/z=1077.5 [M+H⁺]; LCMS method 05.

### Example—91 the Synthetic Route for I-113

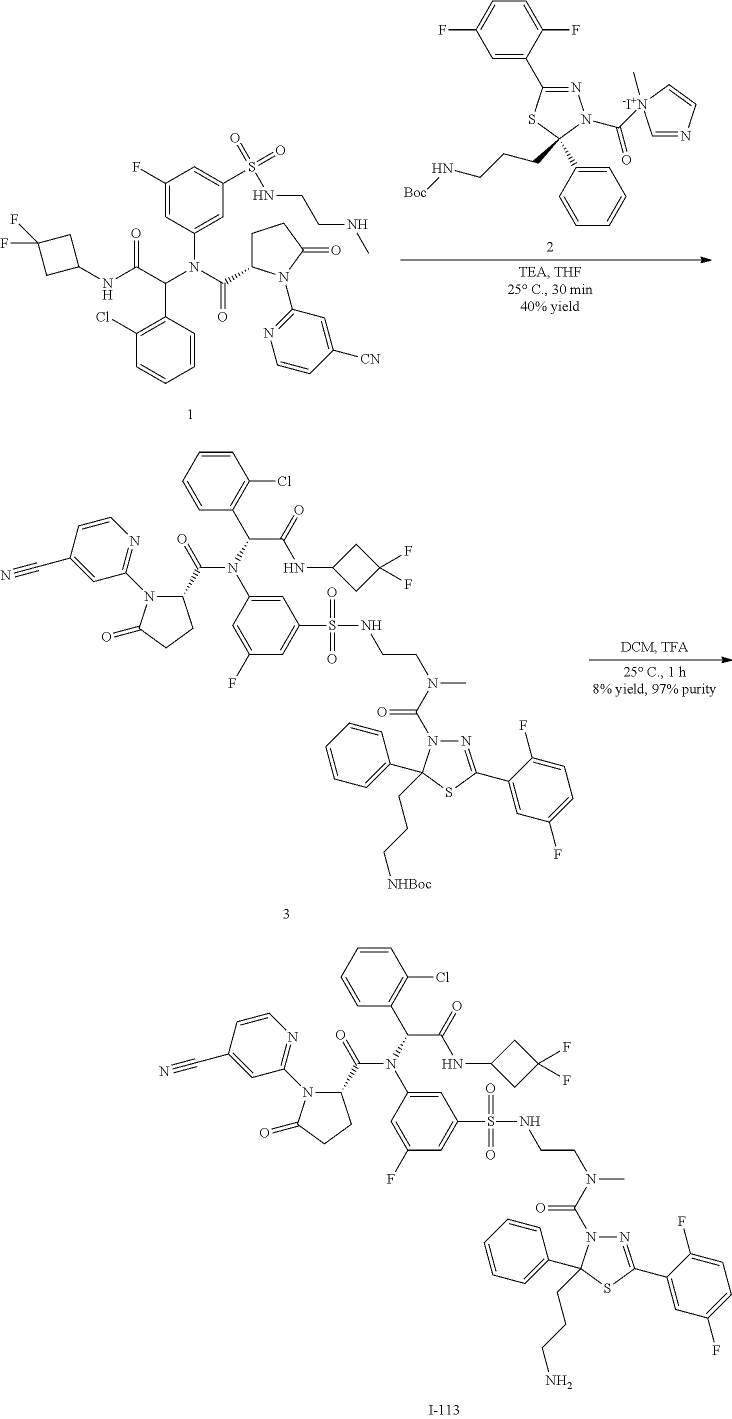

General Information: Synthetic route for compound 2 is described in US2006/247178 A¹.

Step 1. A mixture of (2S)—N-[1-(2-chlorophenyl)-2-[(3,3-difluorocyclobutyl)amino]-2-oxo-ethyl]-1-(4-cyano-2-pyridyl)-N-[3-fluoro-5-[2-(methylamino) ethylsulfamoyl]phenyl]-5-oxo-pyrrolidine-2-carboxamide (45 mg, 63 μmol, 1.0 equiv), tert-butyl N-[3-[(2S)-5-(2,5-difluorophenyl)-3-(1-iodoimidazol-1-ium-1-carbonyl)-2-phenyl-1,3,4-thiadiazol-2-yl]propyl]carbamate (49 mg, 75 μmol, 1.2 equiv), TEA (19 mg, 188 μmol, 26 μL, 3.0 equiv) in THF (0.5 mL) was degassed and purged with N₂ for 3 times, and then the mixture was stirred at 25° C. for 30 min under N₂ atmosphere. The reaction mixture was filtered and concentrated under reduced pressure to give a residue. The residue was purified by prep-HPLC (column: Welch Ultimate C18 150*25 mm*5 μm; mobile phase: [water (FA)-ACN]; B %: 68%-98%, min) to give the tert-butyl N-[3-[3-[2-[3-[[(1R)-1-(2-chlorophenyl)-2-[(3,3-difluorocyclobutyl)amino]-2-oxo-ethyl]-[(2S)-1-(4-cyano-2-pyridyl)-5-oxo-pyrrolidine-2-carbonyl]amino]-5-fluoro-phenyl]sulfonylamino]ethyl-methyl-carbamoyl]-5-(2,5-difluorophenyl)-2-phenyl-1,3,4-thiadiazol-2-yl]propyl]carbamate (30 mg, 25 μmol, 40% yield) as a white solid.

Step 2. A mixture of tert-butyl N-[3-[3-[2-[[3-[(1R)-1-(2-chlorophenyl)-2-[(3,3-difluorocyclobutyl)amino]-2-oxo-ethyl]-[(2S)-1-(4-cyano-2-pyridyl)-5-oxo-pyrrolidine-2-carbonyl]amino]-5-fluoro-phenyl]sulfonylamino]ethyl-methyl-carbamoyl]-5-(2,5-difluorophenyl)-2-phenyl-1,3,4-thiadiazol-2-yl]propyl]carbamate (30 mg, 25 μmol, 1.0 equiv) in DCM (1 mL), TFA (0.3 mL) was degassed and purged with N₂ for 3 times, and then the mixture was stirred at 25° C. for 1 h under N₂ atmosphere. The mixture was concentrated to get the residue. The residue was purified by prep-HPLC (column: Waters xbridge 150*25 mm*10 μm; mobile phase: [water (FA)-ACN]; B %: 28%-58%,8 min) to give the 2-(3-aminopropyl)-N-[2-[[3-[[(1R)-1-(2-chlorophenyl)-2-[(3,3-difluorocyclobutyl)amino]-2-oxo-ethyl]-[(2S)-1-(4-cyano-2-pyridyl)-5-oxo-pyrrolidine-2-carbonyl]amino]-5-fluoro-phenyl]sulfonylamino]ethyl]-5-(2,5-difluorophenyl)-N-methyl-2-phenyl-1,3,4-thiadiazole-3-carboxamide (2 mg, 2 μmol, 8% yield, 97% purity, FA) as a white solid. ¹H NMR: (400 MHZ, DMSO-d₆) δ=8.98-8.79 (m, 1H), 8.71-8.60 (m, 1H), 8.52-8.44 (m, 1H), 8.43-8.28 (m, 1H), 8.57-8.25 (m, 1H), 7.66-6.79 (m, 13H), 6.38-6.19 (m, 1H), 6.42-6.16 (m, 1H), 4.91-4.50 (m, 2H), 4.09-3.89 (m, 3H), 3.35-3.19 (m, 8H), 3.02-2.70 (m, 10H), 2.45-2.36 (m, 1H), 2.14-1.83 (m, 2H), 1.68-1.47 (m, 1H). LC-MS: MS (ES⁺): RT=2.712 min, m/z=1077.5 [M+H⁺]; LCMS method 05.

### Example 92—the Synthetic Route for I-114

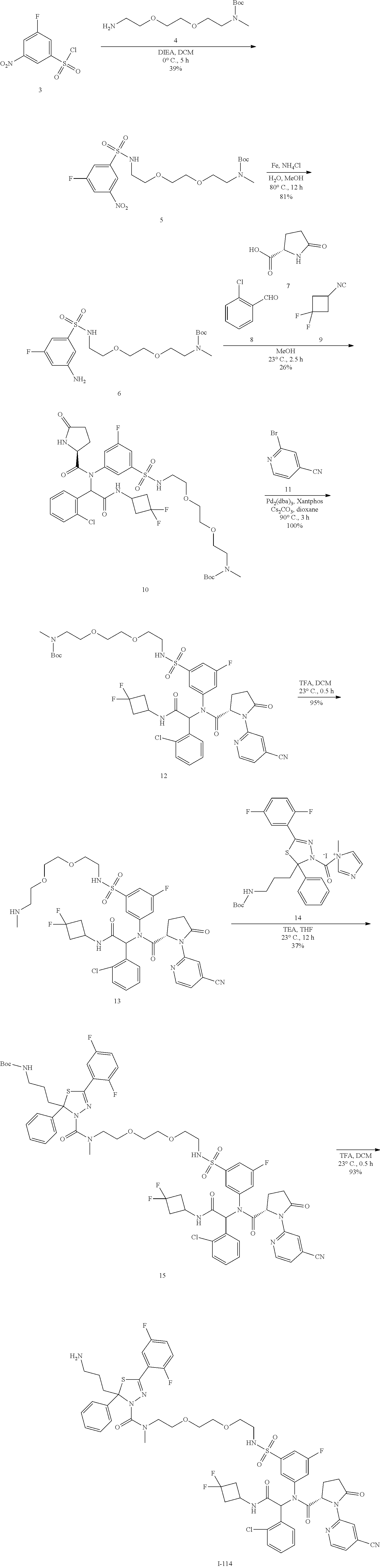

General Information: Synthetic route for compound 3 is described in WO2021/26099. Synthetic route for compound 4 is described in WO2019/195609. Synthetic route for compound 9 is described in WO2013/107405. Synthetic route for compound 14 is described in US2006/247178.

Step 1. To a solution of 3-fluoro-5-nitro-benzenesulfonyl chloride (444 mg, 1.85 mmol, 1.2 equiv) in DCM (5 mL) was added dropwise DIEA (299 mg, 2.32 mmol, 403 μL, 1.5 equiv) at 0° C., and then tert-butyl N-[2-[2-(2-aminoethoxy)ethoxy]ethyl]-N-methyl-carbamate (403 mg, 1.54 mmol, 1.0 equiv) in DCM (2 mL) was added dropwise at 0° C. The resulting mixture was stirred at 0° C. for 5 h. The mixture was filtered and was concentrated to give the residue. The residue was purified by column chromatography (SiO₂, Petroleum ether/Ethyl acetate=5/1 to 1/1). Compound tert-butyl N-[2-[2-[2-[(3-fluoro-5-nitro-phenyl)sulfonylamino]ethoxy]ethoxy]ethyl]-N-methyl-carbamate (283 mg, 608 μmol, 39% yield) was obtained as a colorless oil.

Step 2. A mixture of tert-butyl N-[2-[2-[2-[(3-fluoro-5-nitro-phenyl)sulfonylamino]ethoxy]ethoxy]ethyl]-N-methyl-carbamate (283 mg, 608 μmol, 1.0 equiv), Fe (170 mg, 3.04 mmol, 5.0 equiv) and NH₄Cl (325.20 mg, 6.08 mmol, 10.0 equiv) in MeOH (5 mL) and H₂O (5 mL) was degassed and purged with N₂ for 3 times, and then the mixture was stirred at 80° C. for 2 h under N₂ atmosphere. The mixture was filtered and concentrated to give the residue. The residue was purified by column chromatography (SiO₂, Petroleum ether/Ethyl acetate=3/1 to 1/1). Compound tert-butyl N-[2-[2-[2-[(3-amino-5-fluoro-phenyl)sulfonylamino]ethoxy]ethoxy]ethyl]-N-methyl-carbamate (214 mg, 491 μmol, 81% yield) was obtained as a colorless oil.

Step 3. A mixture of tert-butyl N-[2-[2-[2-[(3-amino-5-fluoro-phenyl)sulfonylamino]ethoxy]ethoxy]ethyl]-N-methyl-carbamate (164 mg, 376 μmol, 1.0 equiv) and 2-chlorobenz aldehyde (53 mg, 376 μmol, 42 μL, 1.0 equiv) in MeOH (5 mL) was stirred at 23° C. for 0.5 h under N₂ atmosphere. Then (2S)-5-oxopyrrolidine-2-carboxylic acid (48.6 mg, 376 μmol, 1.0 equiv) and 1,1-difluoro-3-isocyano-cyclobutane (132 mg, 1.13 mmol, 3.0 equiv) was added. The mixture was stirred at 23° C. for 2 h under N₂ atmosphere. The mixture was concentrated to give the residue. The residue was purified by prep-HPLC (TFA condition column: Phenomenex luna C18 150*25 mm*10 μm; mobile phase: [water (FA)-ACN]; B %: 39%-69%,58 min) to give desired compound tert-butyl N-[2-[2-[2-[[3-[1-(2-chlorophenyl)-2-[(3,3-difluorocyclobutyl)amino]-2-oxo-ethyl]-[(2S)-5-oxopyrrolidine-2-carbonyl]amino]-5-fluoro-phenyl]sulfonylamino]ethoxy]ethoxy]ethyl]-N-methyl-carbamate (80 mg, 99 μmol, 26% yield) as a white solid.

Step 4. A mixture of tert-butyl N-[2-[2-[2-[3-[[1-(2-chlorophenyl)-2-[(3,3-difluorocyclobutyl)amino]-2-oxo-ethyl]-[(2S)-5-oxopyrrolidine-2-carbonyl]amino]-5-fluoro-phenyl]sulfonylamino]ethoxy]ethoxy]ethyl]-N-methyl-carbamate (80 mg, 99 μmol, 1.0 equiv), 2-bromopyridine-4-carbonitrile (21.8 mg, 119 μmol, 1.2 equiv), Pd₂ (dba)3 (9.1 mg, 9.9 μmol, 0.1 equiv), Cs₂CO₃ (97.2 mg, 298 μmol, 3.0 equiv) and Xantphos (11.5 mg, 19.9 μmol, 0.2 equiv) in dioxane (2 mL) was degassed and purged with N₂ for 3 times, and then the mixture was stirred at 90° C. for 3 h under N₂ atmosphere. The mixture was filtered and was concentrated to give the residue. The residue was purified by column chromatography (SiO₂, DCM: MeOH=50:1 to 10:1) to give the desired product. Compound tert-butyl N-[2-[2-[2-[[3-[[1-(2-chlorophenyl)-2-[(3,3-difluorocyclobutyl)amino]-2-oxo-ethyl]-[(2S)-1-(4-cyano-2-pyridyl)-5-oxo-pyrrolidine-2-carbonyl]amino]-5-fluoro-phenyl]sulfonylamino]ethoxy]ethoxy]ethyl]-N-methyl-carbamate (90 mg, 99 μmol, 99% yield) was obtained as a colorless oil.

Step 5. To a solution of tert-butyl N-[2-[2-[2-[[3-[[1-(2-chlorophenyl)-2-[(3,3-difluorocyclobutyl)amino]-2-oxo-ethyl]-[(2S)-1-(4-cyano-2-pyridyl)-5-oxo-pyrrolidine-2-carbonyl]amino]-5-fluoro-phenyl]sulfonylamino]ethoxy]ethoxy]ethyl]-N-methyl-carbamate (90 mg, 99.3 μmol, 1.0 equiv) in DCM (4 mL) was added TFA (3.1 g, 27.0 mmol, 2.0 mL, 272.0 equiv). The mixture was stirred at 23° C. for 0.5 h. The resulting mixture was filtered and concentrated to give the residue. The residue was used for next step without purification. Compound (2S)—N-[1-(2-chlorophenyl)-2-[(3,3-difluorocyclobutyl)amino]-2-oxo-ethyl]-1-(4-cyano-2-pyridyl)-N-[3-fluoro-5-[2-[2-[2-(methylamino)ethoxy]ethoxy]ethylsulfamoyl]phenyl]-5-oxo-pyrrolidine-2-carboxamide (90 mg, 94.9 μmol, 95% yield, 85% purity) was obtained as an orange oil.

Step 6. A mixture of (2S)—N-[(1S)-1-(2-chlorophenyl)-2-[(3,3-difluorocyclobutyl) amino]-2-oxo-ethyl]-1-(4-cyano-2-pyridyl)-N-[3-fluoro-5-[2-[2-[2-(methylamino)ethoxy]ethoxy]ethylsulfamoyl]phenyl]-5-oxo-pyrrolidine-2-carboxamide (10 mg, 10.9 μmol, 1.0 equiv, TFA), tert-butyl N-[3-[(2S)-5-(2,5-difluorophenyl)-3-(1-methylimidazol-1-ium-1-carbonyl)-2-phenyl-1,3,4-thiadiazol-2-yl]propyl]carbamate; iodide (8.7 mg, 13 μmol, 1.2 equiv), TEA (5.5 mg, 54.3 μmol, 7.6 μL, 5.0 equiv) in THF (1 mL) was degassed and purged with N₂ for 3 times, and then the mixture was stirred at 23° C. for 12 h under N₂ atmosphere. The mixture was filtered and concentrated to give the residue. The residue was purified by prep-HPLC (FA condition; column: Phenomenex luna C18 150*25 mm*10 μm; mobile phase: [water (FA)-ACN]; B %: 70%-100%, 10 min) to give the desired product. Compound tert-butyl N-[3-[3-[2-[2-[2-[3-[[1-(2-chlorophenyl)-2-[(3,3-difluorocyclobutyl)amino]-2-oxo-ethyl]-[(2S)-1-(4-cyano-2-pyridyl)-5-oxo-pyrrolidine-2-carbonyl]amino]-5-fluoro-phenyl]sulfonylamino]ethoxy]ethoxy]ethyl-methyl-carbamoyl]-5-(2,5-difluorophenyl)-2-phenyl-1,3,4-thiadiazol-2-yl]propyl]carbamate (50 mg, 36 μmol, 37% yield, TFA) was obtained as a white solid.

Step 7. To a solution of tert-butyl N-[3-[3-[2-[2-[2-[3-[[1-(2-chlorophenyl)-2-[(3,3-difluorocyclobutyl)amino]-2-oxo-ethyl]-[(2S)-1-(4-cyano-2-pyridyl)-5-oxo-pyrrolidine-2-carbonyl]amino]-5-fluoro-phenyl]sulfonylamino]ethoxy]ethoxy]ethyl-methyl-carbamoyl]-5-(2,5-difluorophenyl)-2-phenyl-1,3,4-thiadiazol-2-yl]propyl]carbamate (50.0 mg, 39.5 μmol, 1.0 equiv) in DCM (5 mL) was added TFA (0.5 mL). The mixture was stirred at 23° C. for 0.5 h. The mixture was concentrated to give the residue. The residue was purified by prep-HPLC (FA condition; column: Phenomenex luna C18 150*25 mm*10 μm; mobile phase: [water (FA)-ACN]; B %: 30%-60%, 10 min). Compound 2-(3-aminopropyl)-N-[2-[2-[2-[[3-[[1-(2-chlorophenyl)-2-[(3,3-difluorocyclobutyl)amino]-2-oxo-ethyl]-[(2S)-1-(4-cyano-2-pyridyl)-5-oxo-pyrrolidine-2-carbonyl]amino]-5-fluoro-phenyl]sulfonylamino]ethoxy]ethoxy]ethyl]-5-(2,5-difluorophenyl)-N-methyl-2-phenyl-1,3,4-thiadiazole-3-carboxamide (47.17 mg, 36.85 μmol, 93% yield, 94.66% purity, FA) was obtained as a yellow solid. ¹H NMR: (400 MHZ, CD₃OD) δ=8.83-8.38 (m, 3H), 8.00-7.83 (m, 1H), 7.62-7.16 (m, 12H), 7.10-7.04 (m, 1H), 7.03-6.95 (m, 1H), 6.56-6.40 (m, 1H), 4.89 (m, 2H), 4.30-4.05 (m, 1H), 3.82-3.38 (m, 10H), 3.18-2.71 (m, 11H), 2.66-2.30 (m, 5H), 2.25-2.03 (m, 3H), 1.87-1.71 (m, 1H). LC-MS: MS (ES⁺): RT=2.711 min, m/z=1165.5 [M+H⁺]; LCMS method 05_01.

### Example 93-Cellular Growth Inhibition Assay for HEK293 cells and HeLa cells

Exemplary compounds were tested for ability to inhibit the proliferation of HEK293 cells or HeLa cells. Experimental procedures and results are provided below.

### Part I-Experimental Procedure

HEK293 and HeLa cells were cultured in DMEM medium supplemented with 10% fetal bovine serum and 1% Penn/Strep. Cells were seeded in white 384-well plates at 500 cells/well in 25 mL complete medium. Following seeding, plates were spun at 300×g for three minutes and cultured at 37° C. with 5% CO₂ in a humidified tissue culture incubator.

After 24 hours, compounds were titrated in 100% DMSO and diluted in complete cell culture medium. 25 mL of compound/media mixture was added to cells to bring total volume in well to 50 mL. DMSO alone was used as a negative control. Plates were then spun at 300×g for three minutes and stored at 37° C. with 5% CO₂ for three days.

On Day 0 and Day 3 of compound treatment, cell viability was quantified with CellTiter-Glo 2.0 reagent (Promega). After equilibrating microplates at room temperature for 30 minutes, 25 μL CellTiter-Glo 2.0 reagent was dispensed into each well to bring total volume to 75 mL. Plates were mixed on shaker for 2 minutes at 500 rpm, followed by a 10-minute incubation at room temperature. Following a quick spin, luminescence readings were measured with an En Vision Plate Reader. Data was normalized to DMSO treated Day 0 and Day 3 readings. A four-parameter non-linear regression curve fit was applied to dose-response data in GraphPad Prism data analysis software to determine the half maximal growth inhibitory concentration (GI₅₀) for each compound.

### Part II-Results

Results are provided in Table 3 below for exemplary compounds. The symbol “++++” indicates a GI₅₀ less than 0.5 μM. The symbol “+++” indicates an GI₅₀ in the range of 0.5 μM to 1.5 μM. The symbol “++” indicates a GI₅₀ in the range of greater than 1.5 μM to 5 μM. The symbol “+” indicates a GI₅₀ greater than 5 μM. The symbol “N/A” indicates that no data was available.

TABLE 3

| Compound | CTG HEK293 | CTG HELA |
|---|---|---|
| No. | GI₅₀ | GI₅₀ |
| I-42 | +++ | ++++ |
| I-45 | +++ | +++ |
| I-46 | ++ | ++ |
| I-48 | ++ | ++ |
| I-51 | ++ | +++ |
| I-57 | ++++ | ++++ |
| I-58 | +++ | ++++ |
| I-60 | ++ | +++ |
| I-61 | ++ | +++ |
| I-65 | + | ++ |
| I-64 | + | ++ |
| I-82 | ++ | +++ |
| I-70 | +++ | ++++ |
| I-79 | + | ++ |
| I-69 | ++++ | ++++ |
| I-81 | ++++ | ++++ |
| I-112 | ++ | +++ |
| I-113 | + | + |
| I-111 | ++ | +++ |
| I-114 | + | ++ |
| I-96 | ++++ | ++++ |
| I-97 | ++++ | ++++ |
| I-98 | ++++ | ++++ |
| I-105 | + | + |
| I-106 | + | + |
| I-107 | + | +++ |
| I-108 | + | ++++ |
| I-92 | ++++ | ++++ |
| I-93 | + | ++ |
| I-109 | + | + |
| I-110 | + | + |
| I-89 | ++ | ++++ |
| I-94 | ++ | +++ |
| I-95 | ++ | +++ |
| I-90 | ++++ | ++++ |
| I-91 | ++++ | ++++ |
| I-99 | ++++ | ++++ |
| I-100 | ++++ | ++++ |
| I-101 | ++++ | ++++ |
| I-102 | ++++ | ++++ |
| I-103 | +++ | +++ |
| I-104 | + | + |

### Example 94-Assay for Binding Affinity to BTK

Exemplary compounds were tested for ability to bind to BTK. Experimental procedures and results are provided below.

### Part I—Experimental Procedure

Compounds were tested using a KdELECT assay. Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 111X stocks in 100% DMSO. Kd values were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 mL. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM nonbiotinylated affinity ligand) and incubated at room temperature with shaking for 30 minutes. The kinase concentration in the eluates was measured by qPCR.

### Part II—Results

Results showing ability of exemplary compounds to bind BTK are provided in Table 4 below. The symbol “++++” indicates a Kd less than 0.05 μM. The symbol “+++” indicates an Kd in the range of 0.05 μM to 0.5 μM. The symbol “++” indicates a Kd in the range of greater than 0.5 μM to 2.5 μM. The symbol “+” indicates a Kd greater than 2.5 μM. The symbol “N/A” indicates that no data was available.

TABLE 4

| Compound | |
|---|---|
| No. | Kd |
| I-3 | +++ |
| I-5 | ++++ |
| I-7 | +++ |
| I-8 | +++ |
| I-13 | +++ |
| I-15 | ++++ |
| I-17 | ++ |
| I-18 | ++++ |
| I-24 | ++++ |
| I-26 | ++++ |
| I-28 | ++ |
| I-29 | ++++ |
| I-36 | +++ |
| I-37 | +++ |
| I-39 | ++++ |
| I-42 | ++++ |
| I-43 | ++++ |
| I-44 | ++++ |
| I-47 | ++++ |
| I-48 | ++++ |
| I-51 | ++++ |
| I-52 | ++++ |
| I-53 | ++++ |
| I-64 | ++++ |
| I-82 | +++ |
| I-67 | ++++ |
| I-68 | ++++ |
| I-77 | ++ |
| I-80 | ++++ |
| I-70 | ++++ |
| I-79 | ++++ |
| I-69 | ++++ |
| I-81 | ++++ |

### Example 95-Assay for Binding Affinity to Androgen Receptor

Exemplary compounds were tested for ability to bind to the androgen receptor. Experimental procedures and results are provided below.

### Part I—Experimental Procedure

Fractions of cell cytosol (106 cell/point) were incubated for 24 hr at 4° C. with 1 nM [³H]methyltrienolone in the absence or presence of the test compound in a buffer containing 25 mM Hepes-Tris (pH 7.4), 1 mM EDTA, 10 mM Na₂MoO₄, 2 mM DTT, 5 μM triamcinolone acetonide, and 10% glycerol. Nonspecific binding was determined in the presence of 1 μM testosterone. Following incubation, the samples were filtered rapidly under vacuum through glass fiber filters (GF/B, Packard) presoaked with 0.3% PEI and rinsed several times with ice-cold 50 mM Tris —HCl using a 96-sample cell harvester (Unifilter, Packard). The filters were dried then counted for radioactivity in a scintillation counter (Topcount, Packard) using a scintillation cocktail (Microscint 0, Packard). The results are expressed as a percent inhibition of the control radioligand specific binding. The standard reference compound is testosterone, which is tested in each experiment at several concentrations to obtain a competition curve from which its IC₅₀ is calculated.

### Part II—Results

Results showing ability of exemplary compounds to bind to the androgen receptor are provided in Table 5 below. The symbol “++++” indicates a Kd less than 0.05 μM. The symbol “+++” indicates an Kd in the range of 0.05 μM to 0.5 μM. The symbol “++” indicates a Kd in the range of greater than 0.5 μM to 2.5 μM. The symbol “+” indicates a Kd greater than 2.5 μM. The symbol “N/A” indicates that no data was available.

TABLE 5

| Compound No. | Kd |
|---|---|
| I-21 | ++++ |
| I-34 | ++++ |

### Example 96-Assay for Binding Affinity to BRD4-BD1

Exemplary compounds were tested for ability to bind to BRD4-BD1. Experimental procedures and results are provided below.

### Part I—Experimental Procedure

Compounds were tested using a bromoKdELECT assay. T7 phage strains displaying bromodomains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (5,000×g) and filtered (0.2 μm) to remove cell debris. Streptavidin-coated magnetic beads were treated with biotinylated small molecule or acetylated peptide ligands for 30 minutes at room temperature to generate affinity resins for bromodomain assays. The liganded beads were blocked with excess biotin and washed with blocking buffer (SeaBlock (Pierce), 1% BSA, 0.05% Tween 20, 1 mM DTT) to remove unbound ligand and to reduce nonspecific phage binding. Binding reactions were assembled by combining bromodomains, liganded affinity beads, and test compounds in 1×binding buffer (17% SeaBlock, 0.33×PBS, 0.04% Tween 20, 0.02% BSA, 0.004% Sodium azide, 7.4 mM DTT). Test compounds were prepared as 1000X stocks in 100% DMSO. Kds were determined using an 11-point 3-fold compound dilution series with one DMSO control point. All compounds for Kd measurements are distributed by acoustic transfer (non-contact dispensing) in 100% DMSO. The compounds were then diluted directly into the assays such that the final concentration of DMSO was 0.09%. All reactions performed in polypropylene 384-well plates. Each was a final volume of 0.02 ml. The assay plates were incubated at room temperature with shaking for 1 hour and the affinity beads were washed with wash buffer (1×PBS, 0.05% Tween 20). The beads were then resuspended in elution buffer (1×PBS, 0.05% Tween 20, 2 μM non-biotinylated affinity ligand) and incubated at room temperature with shaking for 30 minutes. The bromodomain concentration in the eluates was measured by qPCR.

### Part II—Results

Results showing ability of exemplary compounds to bind to BRD4-BD1 are provided in Table 6 below. The symbol “++++” indicates a Kd less than 0.05 μM. The symbol “+++” indicates a Kd in the range of 0.05 μM to 0.5 μM. The symbol “++” indicates a Kd in the range of greater than 0.5 μM to 2.5 μM. The symbol “+” indicates a Kd greater than 2.5 μM. The symbol “N/A” indicates that no data was available.

TABLE 6

| Compound No. | Kd |
|---|---|
| I-24 | +++ |
| I-26 | +++ |
| I-28 | + |
| I-29 | ++++ |
| I-32 | ++ |
| I-34 | +++ |

### Example 97-Assay for Binding Affinity to BRD4-BD2

Exemplary compounds were tested for ability to bind to BRD4-BD2. Experimental procedures and results are provided below.

### Part I—Experimental Procedure

Compounds were tested using a bromoKdELECT assay. T7 phage strains displaying bromodomains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (5,000×g) and filtered (0.2 μm) to remove cell debris. Streptavidin-coated magnetic beads were treated with biotinylated small molecule or acetylated peptide ligands for 30 minutes at room temperature to generate affinity resins for bromodomain assays. The liganded beads were blocked with excess biotin and washed with blocking buffer (SeaBlock (Pierce), 1% BSA, 0.05% Tween 20, 1 mM DTT) to remove unbound ligand and to reduce nonspecific phage binding. Binding reactions were assembled by combining bromodomains, liganded affinity beads, and test compounds in 1×binding buffer (17% SeaBlock, 0.33×PBS, 0.04% Tween 20, 0.02% BSA, 0.004% Sodium azide, 7.4 mM DTT). Test compounds were prepared as 1000X stocks in 100% DMSO. Kds were determined using an 11-point 3-fold compound dilution series with one DMSO control point. All compounds for Kd measurements are distributed by acoustic transfer (non-contact dispensing) in 100% DMSO. The compounds were then diluted directly into the assays such that the final concentration of DMSO was 0.09%. All reactions performed in polypropylene 384-well plates. Each was a final volume of 0.02 ml. The assay plates were incubated at room temperature with shaking for 1 hour and the affinity beads were washed with wash buffer (1×PBS, 0.05% Tween 20). The beads were then resuspended in elution buffer (1×PBS, 0.05% Tween 20, 2 μM non-biotinylated affinity ligand) and incubated at room temperature with shaking for 30 minutes. The bromodomain concentration in the eluates was measured by qPCR.

### Part II—Results

Results showing ability of exemplary compounds to bind to BRD4-BD2 are provided in Table 7 below. The symbol “++++” indicates a Kd less than 0.05 μM. The symbol “+++” indicates a Kd in the range of 0.05 μM to 0.5 μM. The symbol “++” indicates a Kd in the range of greater than 0.5 μM to 2.5 μM. The symbol “+” indicates a Kd greater than 2.5 μM. The symbol “N/A” indicates that no data was available.

TABLE 7

| Compound No. | Kd |
|---|---|
| I-24 | ++++ |
| I-26 | ++++ |
| I-28 | ++ |
| I-29 | ++++ |
| I-32 | +++ |
| I-34 | +++ |

### Example 98-Assay for Binding Affinity to CDK1

Exemplary compounds were tested for ability to bind to CDK1. Experimental procedures and results are provided below.

### Part I—Experimental Procedure

Compounds were tested using a KdELECT assay. Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1×binding buffer (20% SeaBlock, 0.17×PBS, 0.05% Tween 20, 6 mM DTT). Test compounds were prepared as 111X stocks in 100% DMSO. Kd values were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 mL. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM nonbiotinylated affinity ligand) and incubated at room temperature with shaking for 30 minutes. The kinase concentration in the eluates was measured by qPCR.

### Part II—Results

Results showing ability of exemplary compounds to bind to CDK1 are provided in Table 8 below. The symbol “++++” indicates a Kd less than 0.05 μM. The symbol “+++” indicates a Kd in the range of 0.05 μM to 0.5 μM. The symbol “++” indicates a Kd in the range of greater than 0.5 μM to 2.5 μM. The symbol “+” indicates a Kd greater than 2.5 μM. The symbol “N/A” indicates that no data was available.

TABLE 8

| Compound No. | Kd |
|---|---|
| I-13 | ++ |
| I-15 | +++ |
| I-18 | ++++ |
| I-43 | + |
| I-47 | + |

### Example 99-Assay for Binding Affinity to CDK9

Exemplary compounds were tested for ability to bind to CDK9. Experimental procedures and results are provided below.

### Part I—Experimental Procedure

Compounds were tested using a KdELECT. Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1×binding buffer (20% SeaBlock, 0.17×PBS, 0.05% Tween 20, 6 mM DTT). Test compounds were prepared as 111X stocks in 100% DMSO. Kd values were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 mL. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM nonbiotinylated affinity ligand) and incubated at room temperature with shaking for 30 minutes. The kinase concentration in the eluates was measured by qPCR.

### Part II—Results

Results showing ability of exemplary compounds to bind to CDK9 are provided in Table 9 below. The symbol “++++” indicates a Kd less than 0.05 μM. The symbol “+++” indicates a Kd in the range of 0.05 μM to 0.5 μM. The symbol “++” indicates a Kd in the range of greater than 0.5 μM to 2.5 M. The symbol “+” indicates a Kd greater than 2.5 μM. The symbol “N/A” indicates that no data was available.

TABLE 9

| Compound No. | Kd |
|---|---|
| I-42 | +++ |
| I-55 | +++ |
| I-56 | +++ |
| I-83 | ++++ |
| I-86 | ++ |
| I-87 | ++++ |
| I-64 | ++ |
| I-82 | ++++ |
| I-67 | ++++ |
| I-68 | ++ |
| I-77 | +++ |
| I-80 | +++ |
| I-70 | +++ |
| I-79 | +++ |
| I-69 | ++++ |
| I-81 | ++++ |
| I-96 | +++ |
| I-97 | +++ |
| I-98 | +++ |
| I-92 | ++ |
| I-93 | ++ |

### Example 100-Assay for Inhibition of KIF11

Exemplary compounds were tested for ability to inhibit KIF11. Experimental procedures and results are provided below.

### Part I—Experimental Procedure

The ATPase rate for Kif11 was monitored using an enzyme-coupled assay. Porcine brain microtubules (Cat #MT002) were purchased from Cytoskeleton (Denver, CO) and polymerized as per manufacturer's instructions and stored at 1 mg/ml at −80° C. GST-tagged Kif11/Eg5 (Cat #EG01-XL) and the Kinesin ELIPA Biochem Kit (Cat #BK060) was also purchased from Cytoskeleton. Taxol, microtubules, 7-methylthioguanosine (MESG), and purine nucleoside phosphorylase (PNP) were added to final concentrations of 15 mM, 0.05 mg/mL, 200 mM, 1U/m, 1 mM in EPLIA Reaction Buffer and allowed to incubate at room temperature for 15 minutes under rocking. Kif11 was then added at a final concentration of 0.025 mM and allowed to incubate at room temperature for an additional 15 minutes. Master mix (18.5 mL) was added to a black 384-well plate (Corning 3575), to which 0.5 mL of DMSO or compound were added to appropriate wells. Plates were incubated at room temperature for 2 hours. ATPase reactions were initiated by addition of 1 mM ATP and monitored by OD340 every 1 minute over the course of 2 hours. The rate of the reaction was taken from the linear part of the resulting curve. IC₅₀ was determined by plotting inhibitor concentration v. ATPase rate.

### Part II—Results

Results showing inhibition of KIF11 by exemplary compounds are provided in Table 10 below. The symbol “++++” indicates a IC₅₀ less than 0.05 μM. The symbol “+++” indicates an IC₅₀ in the range of 0.05 μM to 0.5 μM. The symbol “++” indicates a IC₅₀ in the range of greater than 0.5 μM to 2.5 μM. The symbol “+” indicates a IC₅₀ greater than 2.5 μM. The symbol “N/A” indicates that no data was available.

TABLE 10

| Compound No. | IC₅₀ |
|---|---|
| I-49 | +++ |
| I-50 | ++++ |
| I-52 | ++++ |
| I-53 | +++ |
| I-54 | ++++ |

### Example 101—Assay for Binding Affinity to mTOR

Exemplary compounds were tested for ability to bind to mTOR. Experimental procedures and results are provided below.

### Part I—Experimental Procedure

Compounds were tested using a KdELECT assay. Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1×binding buffer (20% SeaBlock, 0.17×PBS, 0.05% Tween 20, 6 mM DTT). Test compounds were prepared as 111X stocks in 100% DMSO. Kd values were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 mL. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM nonbiotinylated affinity ligand) and incubated at room temperature with shaking for 30 minutes. The kinase concentration in the eluates was measured by qPCR.

### Part II—Results

Results showing ability of exemplary compounds to bind to mTOR are provided in Table 11 below. The symbol “++++” indicates a Kd less than 0.05 μM. The symbol “+++” indicates a Kd in the range of 0.05 μM to 0.5 μM. The symbol “++” indicates a Kd in the range of greater than 0.5 μM to 2.5 μM. The symbol “+” indicates a Kd greater than 2.5 μM. The symbol “N/A” indicates that no data was available.

TABLE 11

| Compound No. | Kd |
|---|---|
| I-2 | ++ |
| I-3 | +++ |
| I-5 | +++ |
| I-7 | +++ |
| I-8 | + |
| I-9 | ++++ |
| I-10 | +++ |

### Example 102-Assay for Binding Affinity to PLK1

An exemplary compound was tested for ability to bind to PLK1. Experimental procedures and results are provided below.

### Part I—Experimental Procedure

Compounds were tested using a KdELECT assay. Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1×binding buffer (20% SeaBlock, 0.17×PBS, 0.05% Tween 20, 6 mM DTT). Test compounds were prepared as 111X stocks in 100% DMSO. Kd values were determined using an 11-point 3-fold compound dilution series with three DMSO control points. All compounds for Kd measurements are distributed by acoustic transfer (non-contact dispensing) in 100% DMSO.

The compounds were then diluted directly into the assays such that the final concentration of DMSO was 0.9%. All reactions performed in polypropylene 384-well plate. Each was a final volume of 0.02 mL. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM nonbiotinylated affinity ligand) and incubated at room temperature with shaking for 30 minutes. The kinase concentration in the eluates was measured by qPCR.

### Part II—Results

Results showing ability of an exemplary compound to bind to PLK1 are provided in Table 12 below. The symbol “++++” indicates a Kd less than 0.05 μM. The symbol “+++” indicates a Kd in the range of 0.05 μM to 0.5 μM. The symbol “++” indicates a Kd in the range of greater than 0.5 μM to 2.5 μM. The symbol “+” indicates a Kd greater than 2.5 μM. The symbol “N/A” indicates that no data was available.

TABLE 12

| Compound No. | Kd |
|---|---|
| I-41 | ++++ |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound represented by Formula I 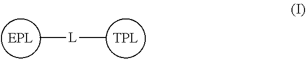
or a pharmaceutically acceptable salt thereof; wherein:
EPL is a moiety that binds to an effector protein selected from mTOR, PLK1, CDK1, CDK2, CDK9, BRD4, AURKA, AURKB, MEK, Src, c-KIT, KIF11, HSP90, tubulin, proteasome, topoisomerase, or HDAC;
L is a linker; and
TPL is a moiety that binds to a target protein selected from BTK, androgen receptor protein, and IDH1.

2. The compound of claim 1, wherein the compound is a compound of Formula I.

3. The compound of claim 1 or 2, wherein the EPL is a moiety that binds to mTOR.

4. The compound of claim 1 or 2, wherein the EPL has the following formula: 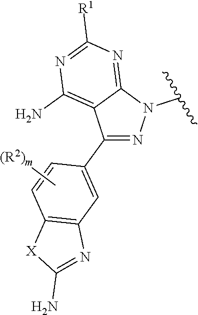
wherein:
R¹ and each R² represent independently for each occurrence halo, C₁-C₆ alkyl, C₁-C₆ haloalkyl, C₃-C₆ cycloalkyl, hydroxyl, C₁-C₆ alkoxy, or cyano;
R³ is hydrogen, C₁-C₆ alkyl, or C₃-C₆ cycloalkyl;
X is O, S, or N(R³); and
m is 0, 1, 2, or 3.

5. The compound of claim 1 or 2, wherein the EPL has the following formula: 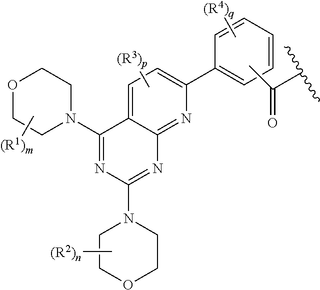
wherein:
R¹ and R² each represent independently for each occurrence halo, C₁-C₆ alkyl, C₁-C₆ haloalkyl, or C₃-C₆ cycloalkyl;
R³ and R⁴ each represent independently for each occurrence halo, C₁-C₆ alkyl, C₁-C₆ haloalkyl, C₃-C₆ cycloalkyl, hydroxyl, C₁-C₆ alkoxy, or cyano;
m, n, and q each represent independently 0, 1, 2, or 3; and
p is 0, 1, or 2.

6. The compound of claim 1 or 2, wherein the EPL is one of the following:

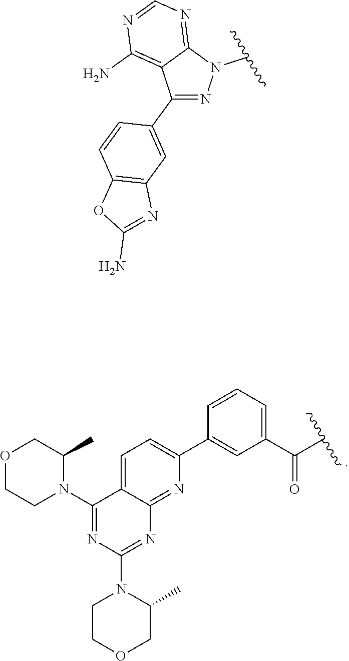 [SMILES: *n1nc(-c2ccc3oc(N)nc3c2)c2c(N)ncnc21 ; *C(=O)c1cccc(-c2ccc3c(N4CCOC[C@H]4C)nc(N4CCOC[C@H]4C)nc3n2)c1]

7. The compound of claim 1 or 2, wherein the EPL is a moiety that binds to PLK1.

8. The compound of claim 1 or 2, wherein the EPL has the following formula: 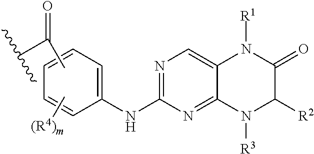
wherein:
R¹ and R² each represent independently C₁-C₆ alkyl, C₁-C₆ haloalkyl, or hydrogen;
R³ is C₃-C₆ cycloalkyl, C₁-C₆ alkyl, C₁-C₆ haloalkyl, or hydrogen;
R⁴ represents independently for each occurrence halo, C₁-C₆ alkyl, C₁-C₆ haloalkyl, C₃-C₆ cycloalkyl, hydroxyl, C₁-C₆ alkoxy, or cyano; and
m is 0, 1, 2, or 3.

9. The compound of claim 1 or 2, wherein the EPL is

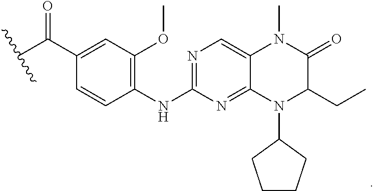 [SMILES: *C(=O)c1ccc(Nc2ncc3c(n2)N(C2CCCC2)C(CC)C(=O)N3C)c(OC)c1]

10. The compound of claim 1 or 2, wherein the EPL has the following formula: 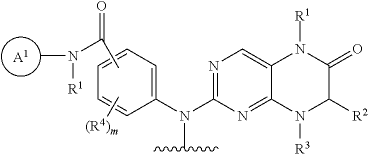
wherein:
R¹ and R² each represent independently for each occurrence C₁-C₆ alkyl, C₁-C₆ haloalkyl, or hydrogen;
R³ is C₃-C₆ cycloalkyl, C₁-C₆ alkyl, C₁-C₆ haloalkyl, or hydrogen;
R⁴ represents independently for each occurrence halo, C₁-C₆ alkyl, C₁-C₆ haloalkyl, C₃-C₆ cycloalkyl, hydroxyl, C₁-C₆ alkoxy, or cyano;
A¹ is a 3-7 membered saturated heterocyclyl containing 1, 2, or 3 heteroatoms independently selected from nitrogen, oxygen, and sulfur, wherein the heterocyclyl is optionally substituted with 1 or 2 occurrences of R⁴; and
m is 0, 1, 2, or 3.

11. The compound of claim 1 or 2, wherein the EPL has the following formula: 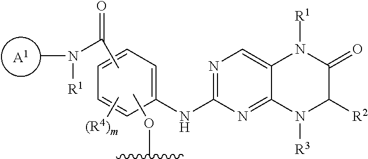
wherein:
R¹ and R² each represent independently for each occurrence C₁-C₆ alkyl, C₁-C₆ haloalkyl, or hydrogen;
R³ is C₃-C₆ cycloalkyl, C₁-C₆ alkyl, C₁-C₆ haloalkyl, or hydrogen;
R⁴ represents independently for each occurrence halo, C₁-C₆ alkyl, C₁-C₆ haloalkyl, C₃-C₆ cycloalkyl, hydroxyl, C₁-C₆ alkoxy, or cyano;
A¹ is a 3-7 membered saturated heterocyclyl containing 1, 2, or 3 heteroatoms independently selected from nitrogen, oxygen, and sulfur, wherein the heterocyclyl is optionally substituted with 1 or 2 occurrences of R⁴; and
m is 0, 1, 2, or 3.

12. The compound of claim 10 or 11, wherein A¹ is piperidinyl substituted with 1 or 2 occurrences of R⁴.

13. The compound of claim 1 or 2, wherein the EPL is one of the following:

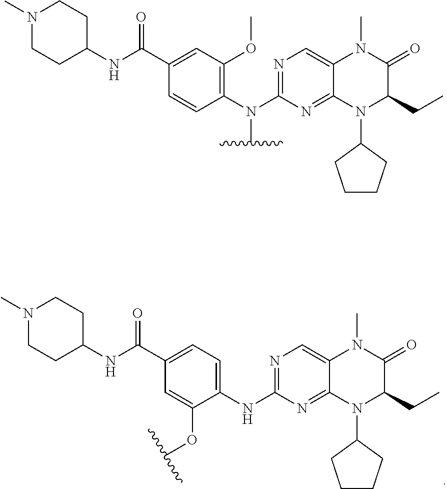

14. The compound of claim 1 or 2, wherein the EPL is a moiety that binds to CDK1.

15. The compound of claim 1 or 2, wherein the EPL has the following formula: 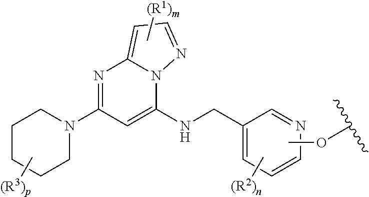
wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

16. The compound of claim 1 or 2, wherein the EPL has the following formula: 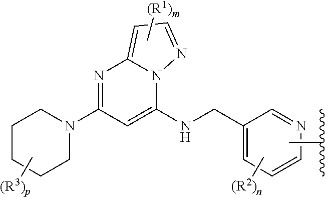
wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

17. The compound of claim 1 or 2, wherein the EPL has the following formula: 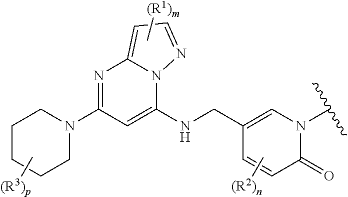
wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

18. The compound of claim 1 or 2, wherein the EPL has the following formula: 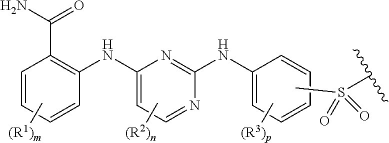
wherein:
R¹, R² and R³ each represent independently for each occurrence halo, C₁-C₆ alkyl, C₁-C₆ haloalkyl, C₃-C₆ cycloalkyl, hydroxyl, C₁-C₆ alkoxy, or cyano;
m and p each represent independently 0, 1, or 2; and
n is 0, 1 or 2

19. The compound of claim 1 or 2, wherein the EPL is one of the following:

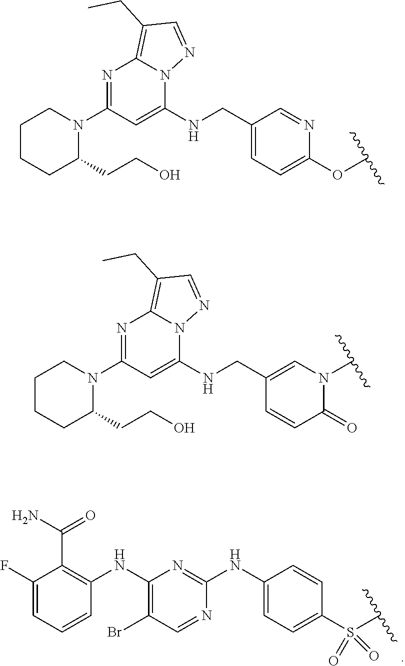 [SMILES: *Oc1ccc(CNc2cc(N3CCCC[C@H]3CCO)nc3c(CC)cnn23)cn1 ; *n1cc(CNc2cc(N3CCCC[C@H]3CCO)nc3c(CC)cnn23)ccc1=O ; *S(=O)(=O)c1ccc(Nc2ncc(Br)c(Nc3cccc(F)c3C(N)=O)n2)cc1]

20. The compound of claim 1 or 2, wherein the EPL is one of the following:

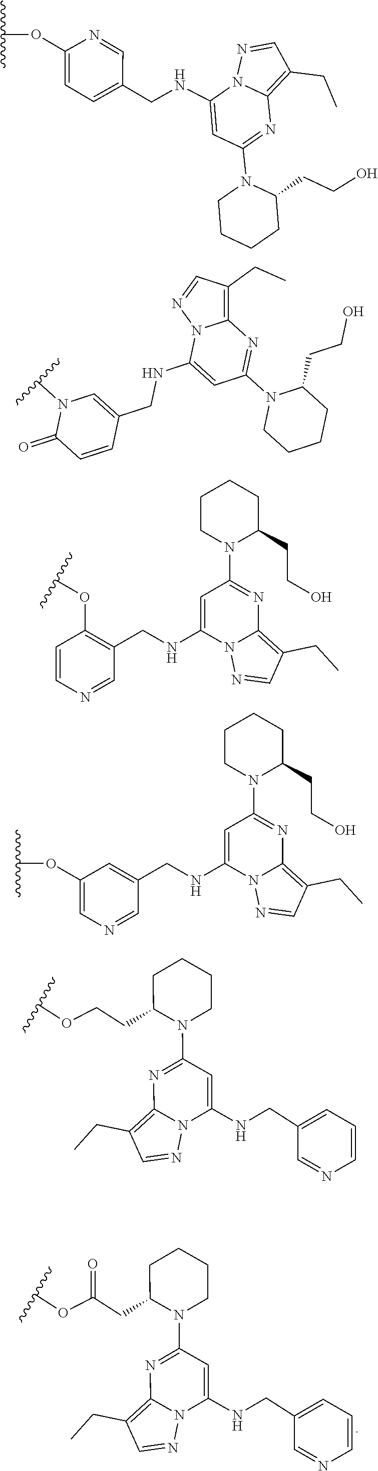

21. The compound of claim 1 or 2, wherein the EPL is

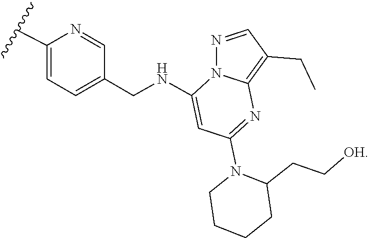 [SMILES: *c1ccc(CNc2cc(N3CCCCC3CCO)nc3c(CC)cnn23)cn1]

22. The compound of claim 1 or 2, wherein the EPL is a moiety that binds to CDK2.

23. The compound of claim 1 or 2, wherein the EPL has the following formula: 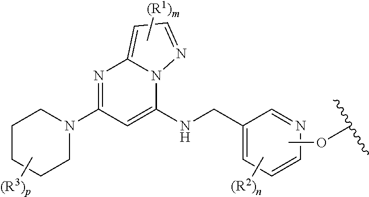
wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

24. The compound of claim 1 or 2, wherein the EPL has the following formula: 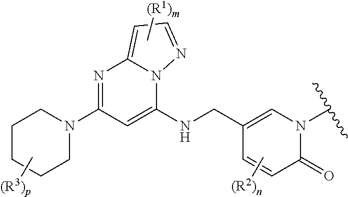
wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

25. The compound of claim 1 or 2, wherein the EPL is one of the following:

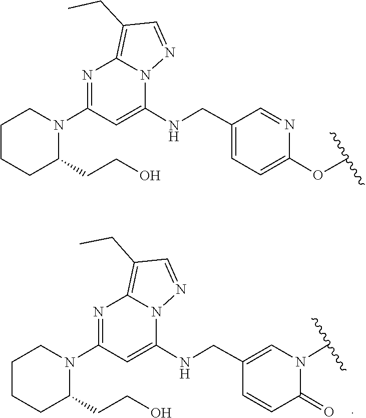 [SMILES: *Oc1ccc(CNc2cc(N3CCCC[C@H]3CCO)nc3c(CC)cnn23)cn1 ; *n1cc(CNc2cc(N3CCCC[C@H]3CCO)nc3c(CC)cnn23)ccc1=O]

26. The compound of claim 1 or 2, wherein the EPL is a moiety that binds to CDK9.

27. The compound of claim 1 or 2, wherein the EPL has the following formula: 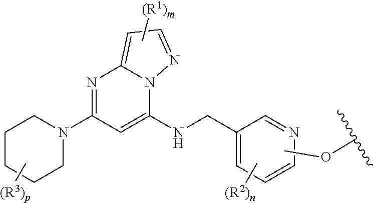
wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

28. The compound of claim 1 or 2, wherein the EPL has the following formula: 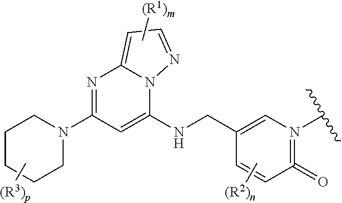
wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ represents independently for each occurrence C₁-C₆ alkyl, C₁-C₆ haloalkyl, C₃-C₆ cycloalkyl, hydroxyl, C₁-C₆ alkoxy, C₁-C₆ hydroxyalkyl, or —(C₁-C₆ alkylene)-(C₁-C₆ alkoxy),
m is 0, 1, or 2; and
n and p each represent independently 0, 1, 2, or 3.

29. The compound of claim 1 or 2, wherein the EPL is one of the following:

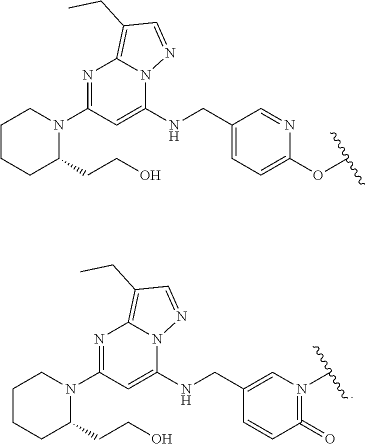

30. The compound of claim 1 or 2, wherein the EPL is a moiety that binds to BRD4.

31. The compound of claim 1 or 2, wherein the EPL has the following formula: 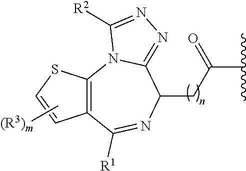
wherein:
R¹ is phenyl, C₃-C₈ cycloalkyl, or 5-6 membered heteroaryl containing 1, 2, or 3 heteroatoms independently selected from nitrogen, oxygen, and sulfur; each of which is optionally substituted with 1, 2, or 3 substituents independently selected from halo, C₁-C₆ alkyl, C₁-C₆ haloalkyl, C₃-C₆ cycloalkyl, hydroxyl, C₁-C₆ alkoxy, or cyano;
R² and each R³ represent independently for each occurrence halo, C₁-C₆ alkyl, C₁-C₆ haloalkyl, C₃-C₆ cycloalkyl, hydroxyl, C₁-C₆ alkoxy, or cyano;
m is 0, 1, or 2; and
n is 0, 1, 2, 3, or 4.

32. The compound of claim 1 or 2, wherein the EPL is

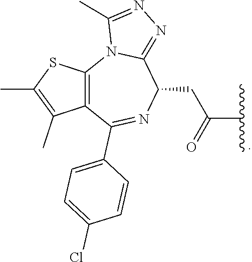 [SMILES: *C(=O)C[C@@H]1N=C(c2ccc(Cl)cc2)c2c(sc(C)c2C)-n2c(C)nnc21]

33. The compound of claim 1 or 2, wherein the EPL has the following formula: 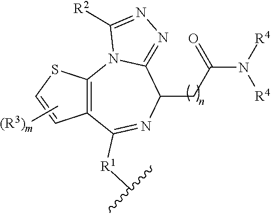
wherein:
R¹ is phenylene substituted with 0, 2, or 3 substituents independently selected from halo, C₁-C₆ alkyl, C₁-C₆ haloalkyl, C₃-C₆ cycloalkyl, hydroxyl, C₁-C₆ alkoxy, or cyano;
R² and R³ each represent independently for each occurrence halo, C₁-C₆ alkyl, C₁-C₆ haloalkyl, C₃-C₆ cycloalkyl, hydroxyl, C₁-C₆ alkoxy, or cyano;
R⁴ represents independently for each occurrence hydrogen, C₁-C₆ alkyl, or C₃-C₆ cycloalkyl;
m is 0, 1, or 2; and
n is 0, 1, 2, 3, or 4.

34. The compound of claim 1 or 2, wherein the EPL is

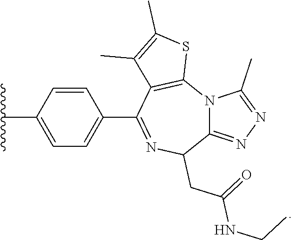 [SMILES: *c1ccc(C2=NC(CC(=O)NCC)c3nnc(C)n3-c3sc(C)c(C)c32)cc1]

35. The compound of claim 1 or 2, wherein the EPL has the following formula: 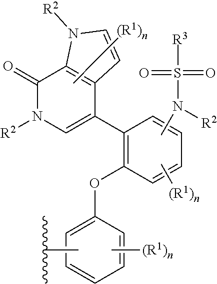
wherein:
R¹ represents independently for each occurrence halo, C₁-C₆ alkyl, C₁-C₆ haloalkyl, C₃-C₆ cycloalkyl, or hydroxyl;
R² represents independently for each occurrence hydrogen, C₁-C₆ alkyl, or C₃-C₆ cycloalkyl;
R³ is C₁-C₆ alkyl or C₃-C₆ cycloalkyl; and
n represents independently for each occurrence 0, 1, 2, 3, or 4.

36. The compound of claim 1 or 2, wherein the EPL is

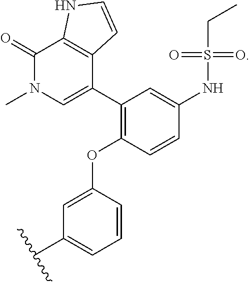 [SMILES: *c1cccc(Oc2ccc(NS(=O)(=O)CC)cc2-c2cn(C)c(=O)c3[nH]ccc23)c1]

37. The compound of claim 1 or 2, wherein the EPL is a moiety that binds to AURKA.

38. The compound of claim 1 or 2, wherein the EPL has the following formula: 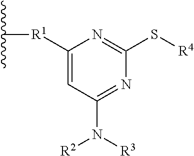
wherein:
R¹ is 4-7 membered, saturated heterocyclylene containing 1, 2, or 3 heteroatoms independently selected from nitrogen, oxygen, and sulfur;
R² is a 5-6membered heteroaryl containing 1, 2, or 3 heteroatoms independently selected from nitrogen, oxygen, and sulfur; wherein the heteroaryl is optionally substituted with 1 or 2 substituents independently selected from halo, C₁-C₆ alkyl, C₁-C₆ haloalkyl, C₃-C₆ cycloalkyl, hydroxyl, C₁-C₆ alkoxy, or cyano;
R³ represents independently for each occurrence H or C₁-C₆ alkyl; and
R⁴ is phenyl or a 5-6 membered heteroaryl containing 1, 2, or 3 heteroatoms independently selected from nitrogen, oxygen, and sulfur; each of which is optionally substituted with 1, 2, or 3 substituents independently selected from —N(R³)C(O)—(C₃-C₆ cycloalkyl), —N(R³)C(O)—(C₁-C₆ alkyl), halo, C₁-C₆ alkyl, C₁-C₆ haloalkyl, C₃-C₆ cycloalkyl, hydroxyl, C₁-C₆ alkoxy, or cyano.

39. The compound of claim 1 or 2, wherein the EPL has the following formula: 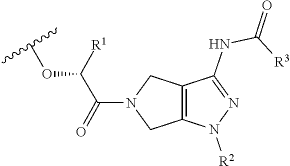
wherein:
R¹ is phenyl or a 5-6 membered heteroaryl containing 1, 2, or 3 heteroatoms independently selected from nitrogen, oxygen, and sulfur; each of which is optionally substituted with 1, 2, or 3 substituents independently selected from halo, C₁-C₆ alkyl, C₁-C₆ haloalkyl, C₃-C₆ cycloalkyl, hydroxyl, C₁-C₆ alkoxy, or cyano;
R² is -(phenylene)-(4-7 membered, saturated heterocyclyl containing 1, 2, or 3 heteroatoms independently selected from nitrogen, oxygen, and sulfur); wherein the heterocyclyl is optionally substituted with 1 or 2 substituents independently selected from halo, C₁-C₆ alkyl, C₁-C₆ haloalkyl, C₃-C₆ cycloalkyl, hydroxyl, C₁-C₆ alkoxy, or cyano;
R³ represents independently for each occurrence H or C₁-C₆ alkyl.

40. The compound of claim 1 or 2, wherein the EPL is one of the following:

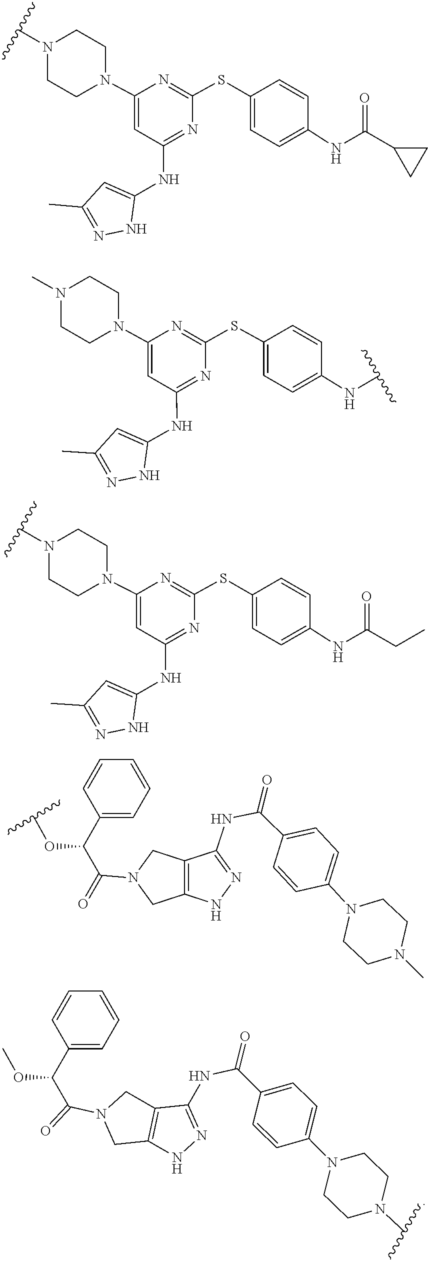

41. The compound of claim 1 or 2, wherein the EPL is a moiety that binds to AURKB.

42. The compound of claim 1 or 2, wherein the EPL has the following formula: 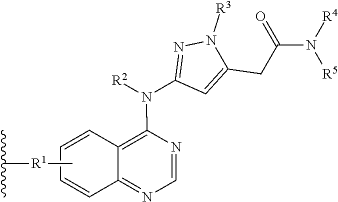
wherein:
R¹ is 4-10 membered heteroalkylene;
R², R³, and R⁵ are independently H or C₁-C₆ alkyl; and
R⁴ is phenyl or a 5-6 membered heteroaryl containing 1, 2, or 3 heteroatoms independently selected from nitrogen, oxygen, and sulfur; each of which is optionally substituted with 1, 2, or 3 substituents independently selected from halo, C₁-C₆ alkyl, C₁-C₆ haloalkyl, C₃-C₆ cycloalkyl, hydroxyl, C₁-C₆ alkoxy, or cyano.

43. The compound of claim 1 or 2, wherein the EPL is one of the following:

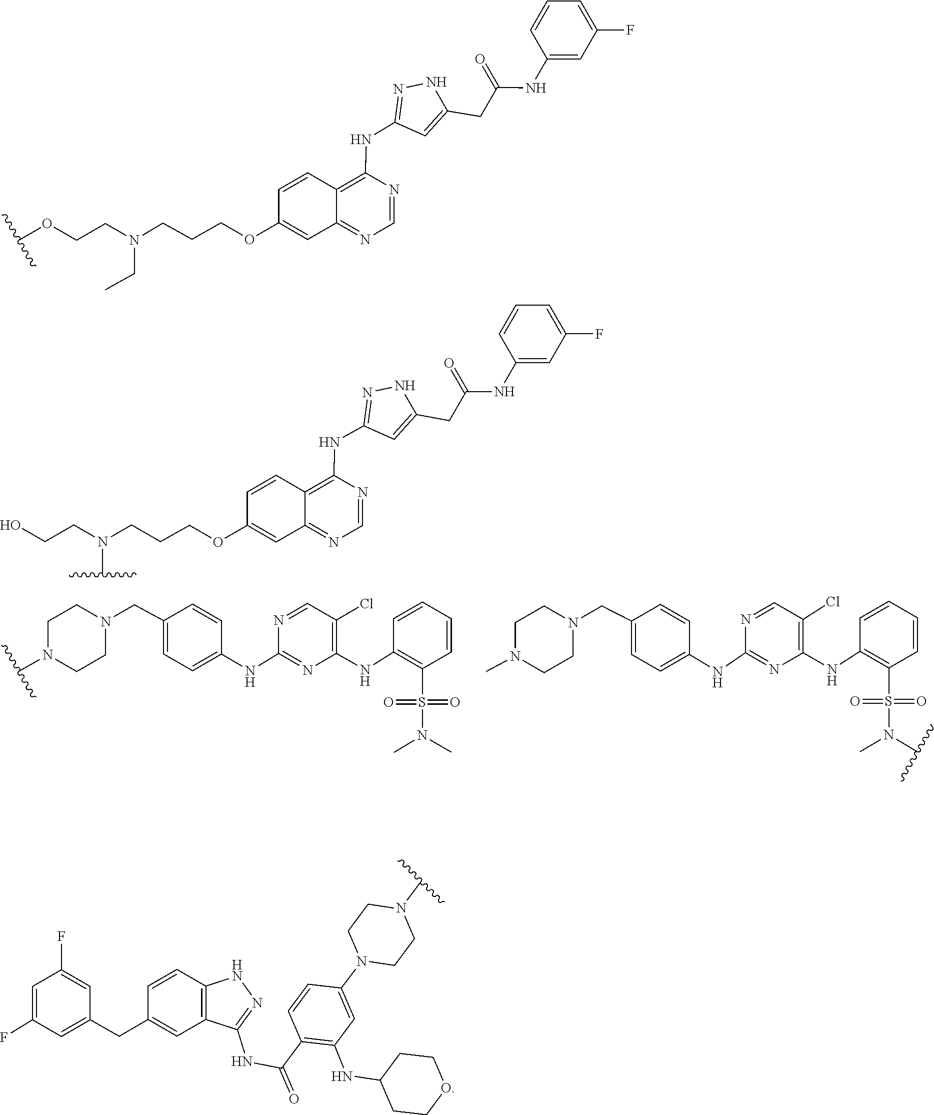

44. The compound of claim 1 or 2, wherein the EPL is a moiety that binds to MEK.

45. The compound of claim 1 or 2, wherein the EPL has the following formula: 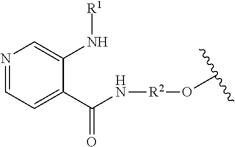
wherein:
R¹ is phenyl or a 5-6 membered heteroaryl containing 1, 2, or 3 heteroatoms independently selected from nitrogen, oxygen, and sulfur; each of which is optionally substituted with 1, 2, or 3 substituents independently selected from halo, C₁-C₆ alkyl, C₁-C₆ haloalkyl, C₃-C₆ cycloalkyl, hydroxyl, C₁-C₆ alkoxy, or cyano; and
R² is —(C₂₋₆ alkylene optionally substituted by one hydroxyl).

46. The compound of claim 1 or 2, wherein the EPL is one of the following:

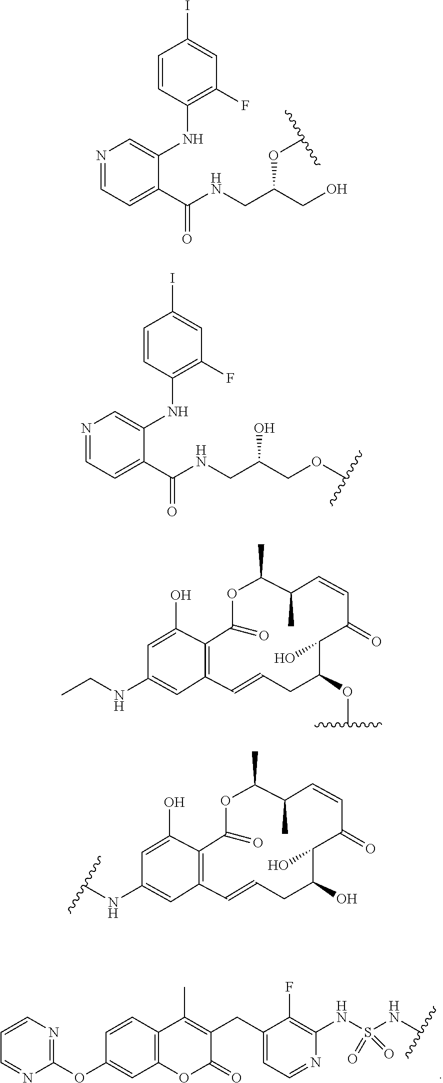

47. The compound of claim 1 or 2, wherein the EPL is a moiety that binds to Src.

48. The compound of claim 1 or 2, wherein the EPL is a moiety that binds to c-KIT.

49. The compound of claim 1 or 2, wherein the EPL is a moiety that binds to KIF11.

50. The compound of claim 1 or 2, wherein the EPL has the following formula: 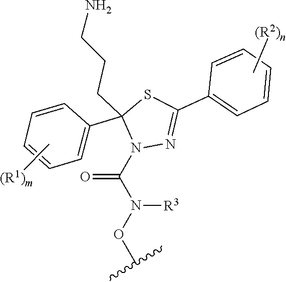
wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ is H, C₁-C₆ alkyl, C₁-C₆ haloalkyl, or C₃-C₆ cycloalkyl; and
m and n each represent independently 0, 1, 2, or 3.

51. The compound of claim 1 or 2, wherein the EPL has the following formula: 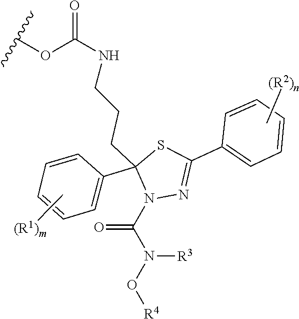
wherein:
R¹ and R² each represent independently for each occurrence halo, C₁-C₆ alkyl, C₁-C₆ haloalkyl, C₃-C₆ cycloalkyl, hydroxyl, C₁-C₆ alkoxy, or cyano;
R³ and R⁴ each represent independently H, C₁-C₆ alkyl, C₁-C₆ haloalkyl, or C₃-C₆ cycloalkyl; and
m and n each represent independently 0, 1, 2, or 3.

52. The compound of claim 1 or 2, wherein the EPL is one of the following:

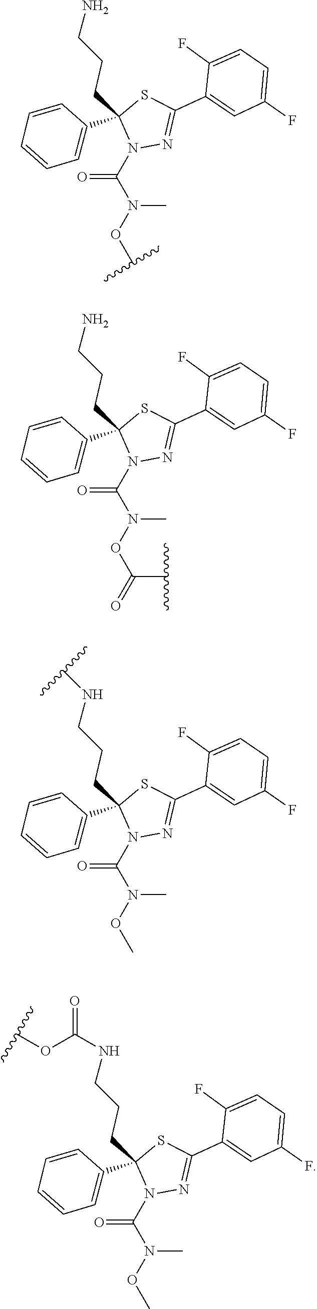 [SMILES: *ON(C)C(=O)N1N=C(c2cc(F)ccc2F)S[C@@]1(CCCN)c1ccccc1 ; *C(=O)ON(C)C(=O)N1N=C(c2cc(F)ccc2F)S[C@@]1(CCCN)c1ccccc1 ; *NCCC[C@@]1(c2ccccc2)SC(c2cc(F)ccc2F)=NN1C(=O)N(C)OC ; *OC(=O)NCCC[C@@]1(c2ccccc2)SC(c2cc(F)ccc2F)=NN1C(=O)N(C)OC]

53. The compound of claim 1 or 2, wherein the EPL is

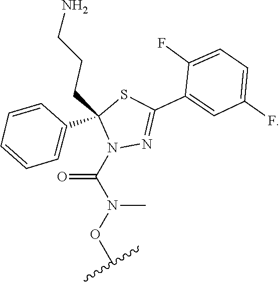 [SMILES: *ON(C)C(=O)N1N=C(c2cc(F)ccc2F)S[C@@]1(CCCN)c1ccccc1]

54. The compound of claim 1 or 2, wherein the EPL is a moiety that binds to HSP90.

55. The compound of claim 1 or 2, wherein the EPL is a moiety that binds to tubulin.

56. The compound of claim 1 or 2, wherein the EPL is one of the following: 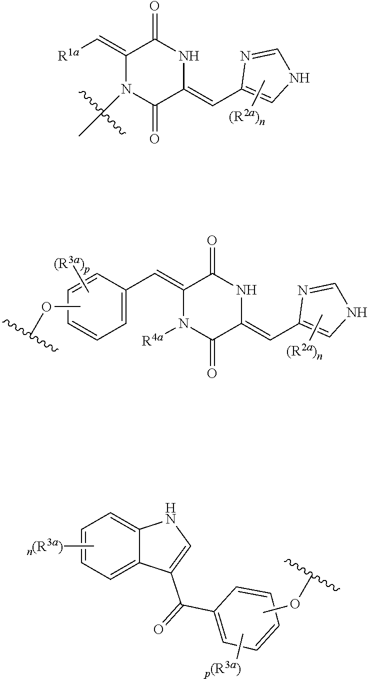
wherein:
R^{1a} is phenyl, C₃-C₆ cycloalkyl, or 5-6 membered heteroaryl containing 1 or 2 heteroatoms independently selected from oxygen, nitrogen, and sulfur; wherein the phenyl, cycloalkyl, and heteroaryl are substituted with 0, 1, 2, or 3 groups independently selected from C₁-C₆ alkyl, C₁-C₆ haloalkyl, C₃-C₆ cycloalkyl, halogen, hydroxyl, C₁-C₆ alkoxy, or —C(O)—(5-6 membered heteroaryl containing 1 or 2 heteroatoms independently selected from oxygen, nitrogen, and sulfur, wherein the heteroaryl is substituted with 0, 1, 2, or 3 groups independently selected from C₁-C₆ alkyl, C₁-C₆ haloalkyl, C₃-C₆ cycloalkyl, halogen, hydroxyl, or C₁-C₆ alkoxy);
R^{2a} each represent independently for each occurrence C₁-C₆ alkyl, C₁-C₆ haloalkyl, or C₃-C₆ cycloalkyl;
R^{3a} represents independently for each occurrence C₁-C₆ alkyl, C₁-C₆ haloalkyl, C₃-C₆ cycloalkyl, halogen, hydroxyl, or C₁-C₆ alkoxy;
R^{4a} is hydrogen, C₁-C₆ alkyl, or C₃-C₆ cycloalkyl;
n and p are independently 0, 1, or 2.

57. The compound of claim 1 or 2, wherein the EPL is one of the following:

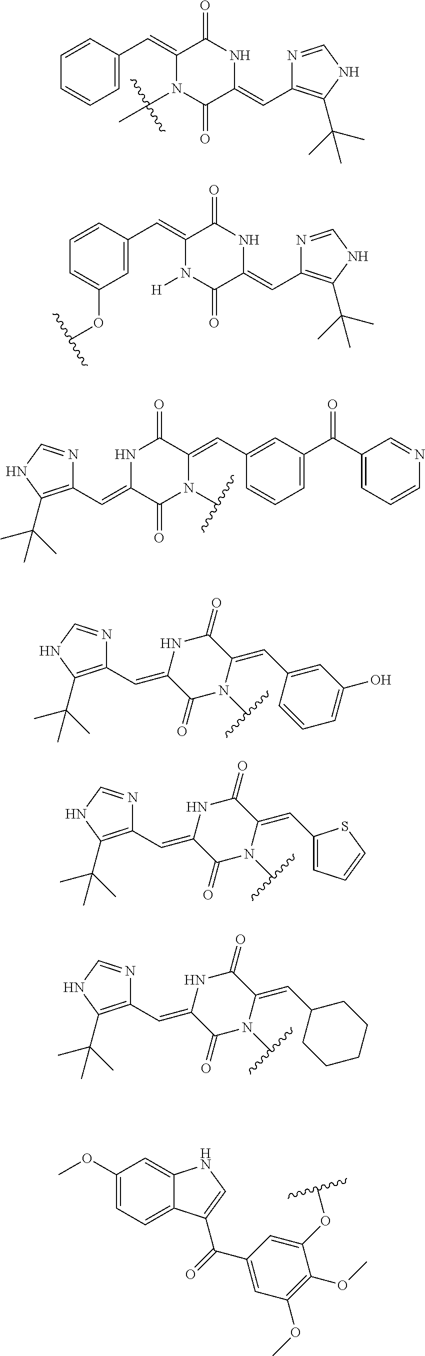

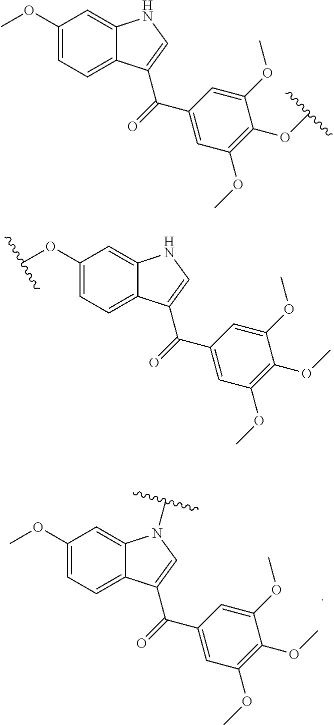

58. The compound of claim 1 or 2, wherein the EPL is a moiety that binds to proteasome.

59. The compound of claim 1 or 2, wherein the EPL is a moiety that binds to topoisomerase.

60. The compound of claim 1 or 2, wherein the EPL is a moiety that binds to HDAC.

61. The compound of any one of claims 1-60, wherein the TPL is a moiety that binds to BTK.

62. The compound of any one of claims 1-60, wherein the TPL is one of the following: 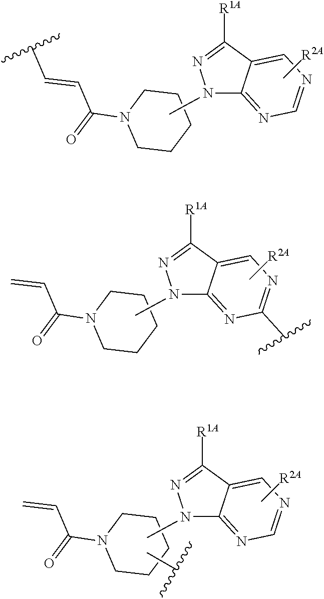
wherein:
R^{1A} is -(phenyl optionally substituted with 1, 2, 3, 4, or 5 substituents independently selected from halo, hydroxyl, C₁₋₄ alkyl, and C₁₋₄ alkoxyl)-O-(phenyl optionally substituted with 1, 2, 3, 4, or 5 substituents independently selected from halo, hydroxyl, C₁₋₄ alkyl, and C₁₋₄ alkoxyl);
R^{2A} is hydrogen, halo, hydroxyl, C₁₋₄ alkyl, C₁₋₄ alkoxyl, or —N(R^{5A})(R^{6A}); and
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to the same nitrogen atom are taken together with the nitrogen atom to which they are attached to form a 3-7 membered heterocyclic ring.

63. The compound of any one of claims 1-60, wherein the TPL is 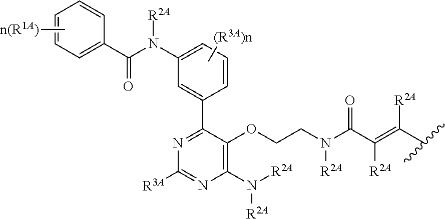
wherein:
R^{1A} and R^{3A} each represent independently for each occurrence hydrogen, halo, hydroxyl, C₁₋₄ alkyl, C₁₋₄ alkoxyl, or C₃₋₆ cycloalkyl;
R^{2A} is hydrogen, C₁₋₆ alkyl, or C₃₋₆ cycloalkyl; and
n and m are independently 1 or 2.

64. The compound of any one of claims 1-60, wherein the TPL is one of the following:

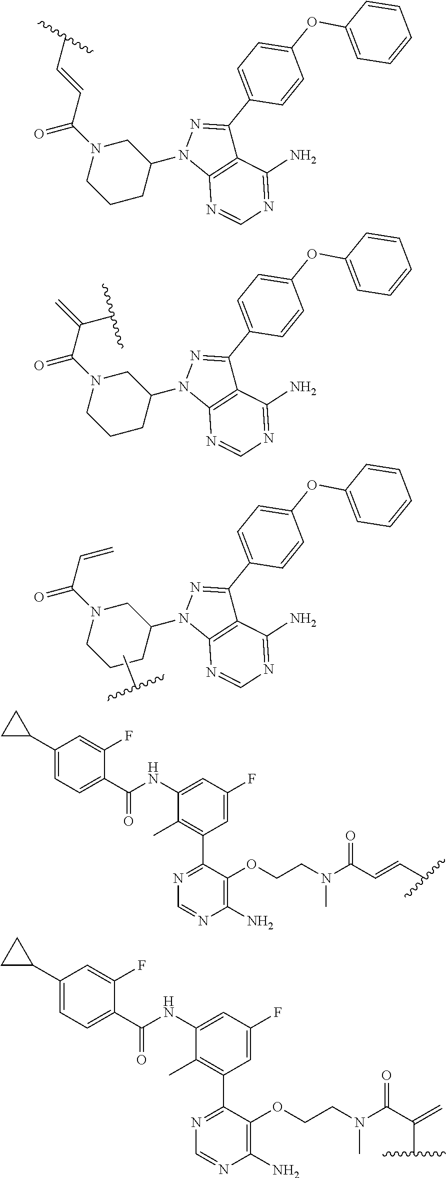

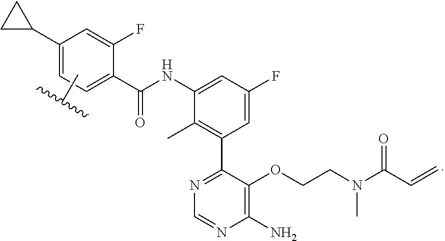

65. The compound of any one of claims 1-60, wherein the TPL is a moiety that binds to androgen receptor protein.

66. The compound of any one of claims 1-60, wherein the TPL is 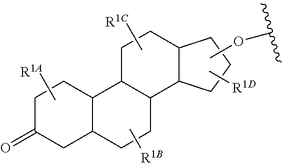
wherein R^{1A}, R^{1B}, R^{1C}, and R^{1D} are independently hydrogen, halo, hydroxyl, or C₁₋₄ alkyl.

67. The compound of any one of claims 1-60, wherein the TPL is

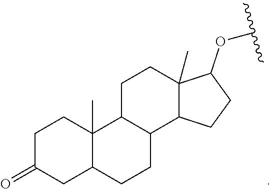 [SMILES: *OC1CCC2C3CCC4CC(=O)CCC4(C)C3CCC12C]

68. The compound of any one of claims 1-60, wherein the TPL is one of the following: 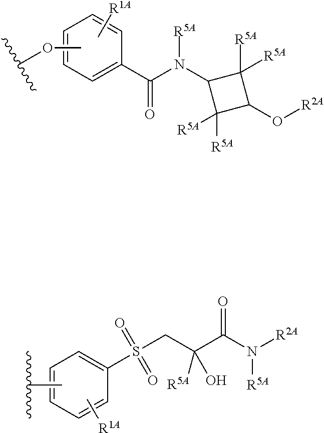
wherein:
R^{1A} is hydrogen, halo, hydroxyl, C₁₋₄ alkyl, C₁₋₄ alkoxyl, or —N(R^{5A})(R^{6A});
R^{2A} is -(phenyl or 5-6 membered heteroaryl containing 1, 2, or 3 heteroatoms independently selected from oxygen, nitrogen, and sulfur, wherein the phenyl and heteroaryl are optionally substituted with 1, 2, 3, 4, or 5 substituents independently selected from halo, cyano, hydroxyl, C₁₋₄ alkyl, C₁₋₄ haloalkyl, and C₁₋₄ alkoxyl); and
R^{5A} and R^{6A} each represent independently for each occurrence hydrogen, C₁₋₄ alkyl, C₃₋₇ cycloalkyl, or —(C₁₋₄ alkylene)-C₃₋₇ cycloalkyl; or an occurrence of R^{5A} and R^{6A} attached to the same nitrogen atom are taken together with the nitrogen atom to which they are attached to form a 3-7 membered heterocyclic ring.

69. The compound of any one of claims 1-60, wherein the TPL is one following:

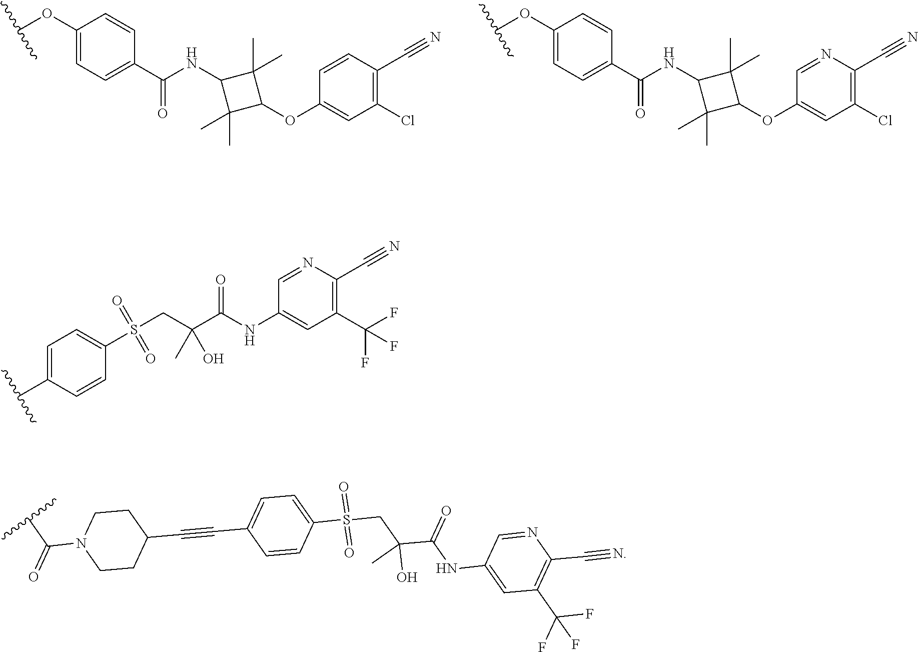 [SMILES: *Oc1ccc(C(=O)NC2C(C)(C)C(Oc3ccc(C#N)c(Cl)c3)C2(C)C)cc1 ; *Oc1ccc(C(=O)NC2C(C)(C)C(Oc3cnc(C#N)c(Cl)c3)C2(C)C)cc1 ; *c1ccc(S(=O)(=O)CC(C)(O)C(=O)Nc2cnc(C#N)c(C(F)(F)F)c2)cc1 ; *C(=O)N1CCC(C#Cc2ccc(S(=O)(=O)CC(C)(O)C(=O)Nc3cnc(C#N)c(C(F)(F)F)c3)cc2)CC1]

70. The compound of any one of claims 1-60, wherein the TPL is a moiety that binds to IDH1.

71. The compound of any one of claims 1-60, wherein the TPL is 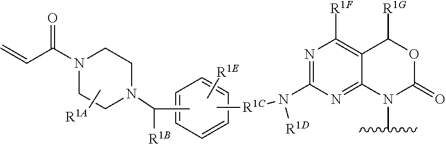
wherein:
R^{1A} and R^{1D} are independently hydrogen or C₁₋₄ alkyl;
R^{1B} is hydrogen, C₁₋₄ alkyl, or (C₁₋₄ alkylene)-(C₃₋₆ cycloalkyl);
R^{1C} is C₁₋₄ alkylene;
R^{1E}, R^{1F}, and R^{1G} are independently hydrogen, halo, hydroxyl, C₁₋₄ alkyl, or C₁₋₄ alkoxyl.

72. The compound of any one of claims 1-60, wherein the TPL is

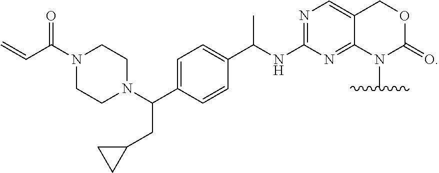 [SMILES: *N1C(=O)OCc2cnc(NC(C)c3ccc(C(CC4CC4)N4CCN(C(=O)C=C)CC4)cc3)nc21]

73. The compound of any one of claims 1-60, wherein the TPL is one of the following: 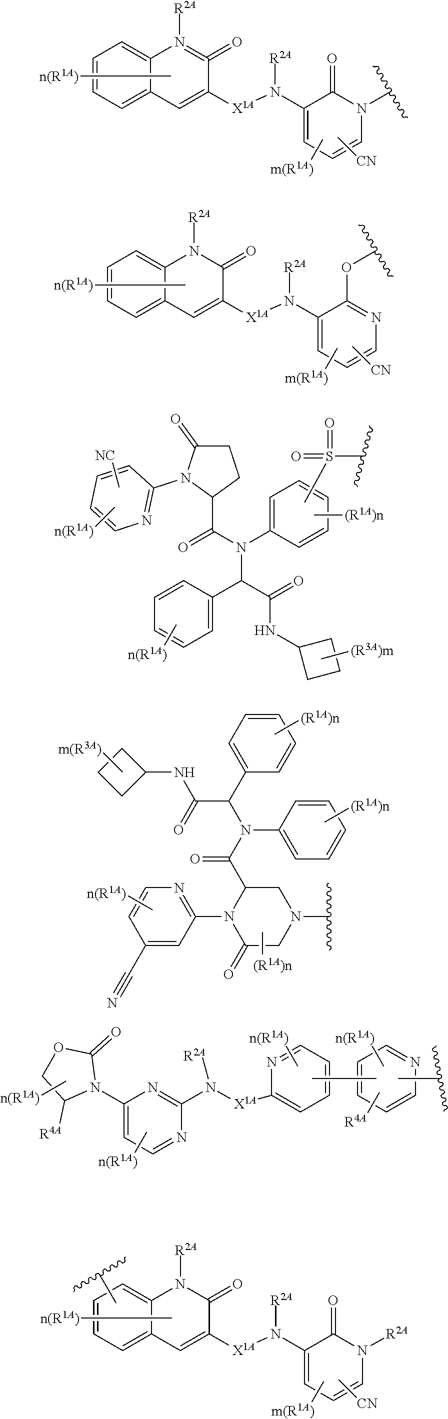
wherein:
R¹⁴ and R³⁴ each represent independently for each occurrence hydrogen, halo, hydroxyl, C₁₋₄ alkyl, C₁₋₄ alkoxyl, or C₃₋₆ cycloalkyl;
R²⁴ is hydrogen, C₁₋₆ alkyl, or C₃₋₆ cycloalkyl;
R⁴⁴ is C₁₋₄ haloalkyl;
X^{1A} is C₁₋₆ alkylene; and
n and m each represent independently for each occurrence 1 or 2.

74. The compound of any one of claims 1-60, wherein the TPL is one of the following:

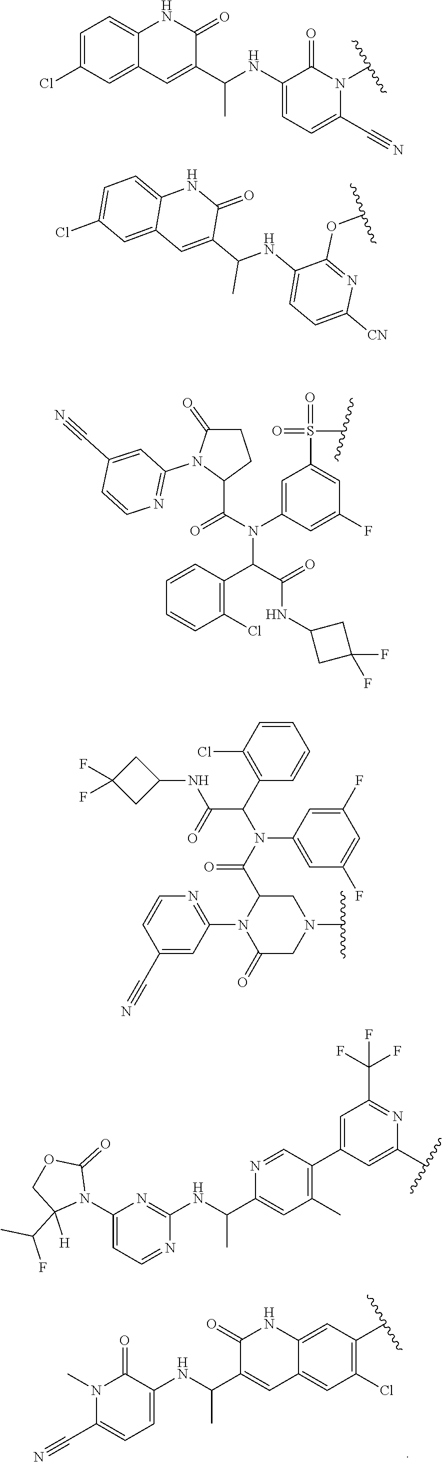 [SMILES: *n1c(C#N)ccc(NC(C)c2cc3cc(Cl)ccc3[nH]c2=O)c1=O ; *Oc1nc(C#N)ccc1NC(C)c1cc2cc(Cl)ccc2[nH]c1=O ; *S(=O)(=O)c1cc(F)cc(N(C(=O)C2CCC(=O)N2c2cc(C#N)ccn2)C(C(=O)NC2CC(F)(F)C2)c2ccccc2Cl)c1 ; *N1CC(=O)N(c2cc(C#N)ccn2)C(C(=O)N(c2cc(F)cc(F)c2)C(C(=O)NC2CC(F)(F)C2)c2ccccc2Cl)C1 ; *c1cc(-c2cnc(C(C)Nc3nccc(N4C(=O)OCC4C(C)F)n3)cc2C)cc(C(F)(F)F)n1 ; *c1cc2[nH]c(=O)c(C(C)Nc3ccc(C#N)n(C)c3=O)cc2cc1Cl]

75. The compound of any one of claims 1-74, wherein L is a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)2—, —N(H)S(O)2—, —N(C₁₋₆ alkyl)S(O)2—, —S(O)2N(H)—, —S(O)2N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur.

76. The compound of any one of claims 1-74, wherein L is a bivalent, saturated or unsaturated, straight or branched C₁₋₆₀ hydrocarbon chain, wherein (i)0-20 methylene units of the hydrocarbon are independently replaced with —O—, —S—, —N(H)—, —N(C₁₋₆ alkyl)-, —OC(O)—, —C(O)O—, —S(O)—, —S(O)2—, —N(H)S(O)2—, —N(C₁₋₆ alkyl)S(O)2—, —S(O)2N(H)—, —S(O)2N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur, and (ii)0-1 methylene units of the hydrocarbon are independently replaced with —C(O)—(C₂₋₆ alkenylene)-, —C(O)—(C₂₋₆ fluoroalkenylene)-, —C(O)—(C₂₋₆ alkynylene)-, —S(O)2-(C₂₋₆ alkenylene)-, —S(O)2-(C₂₋₆ fluoroalkenylene)-, —S(O)2-(C₂₋₆ alkynylene)-, or —(C₁₋₆ alkylene substituted with one R^{WH})—, wherein R^{WH} is

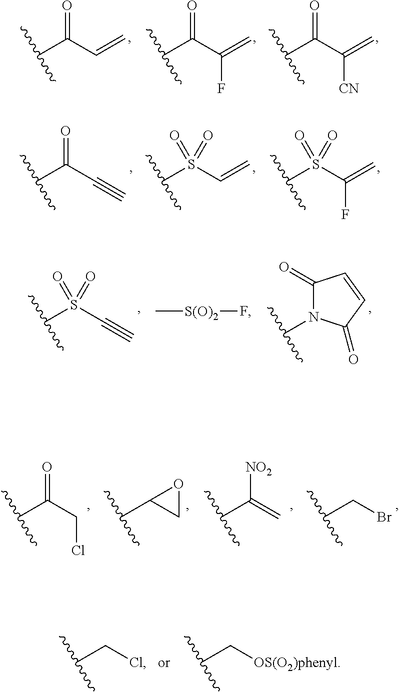

77. The compound of any one of claims 1-74, wherein L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, —C(O)N(C₁₋₆ alkyl)-, 3-10 membered carbocyclyl, or 3-10 membered heterocyclyl containing 1, 2, or 3 heteroatoms independently selected from nitrogen, oxygen, and sulfur.

78. The compound of any one of claims 1-74, wherein L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, or —C(O)N(C₁₋₆ alkyl)-.

79. The compound of any one of claims 1-74, wherein L has the formula-(C₀₋₁₂ alkylene)-(optionally substituted 3-40 membered heteroalkylene)-(C₀₋₁₂ alkylene)-.

80. The compound of any one of claims 1-74, wherein L is C₄₋₁₄ alkylene.

81. The compound of any one of claims 1-74, wherein L is —(CH₂)₆₋₁₀—.

82. The compound of any one of claims 1-74, wherein L is —CH₂CH₂ (OCH₂CH₂)—*** F, —CH₂CH₂ (OCH₂CH₂)₂—***, —CH₂CH₂ (OCH₂CH₂)₃—***, —CH₂CH₂ (OCH₂CH₂)₄—***, —CH₂CH₂ (OCH₂CH₂)₅—***, —CH₂CH₂ (OCH₂CH₂)₆—***, —CH₂CH₂ (OCH₂CH₂)₇—***, —CH₂CH₂ (OCH₂CH₂)₈—***, —CH₂CH₂ (OCH₂CH₂)₉—***, —CH₂CH₂ (OCH₂CH₂)₁₀—** *, —CH₂CH₂ (OCH₂CH₂)₁₁—***, —CH₂CH₂ (OCH₂CH₂)12—***, —CH₂CH₂ (OCH₂CH₂)₁₃—***, —CH₂CH₂ (OCH₂CH₂)₁₄—***, —CH₂CH₂ (OCH₂CH₂)₁₅—***, or —CH₂CH₂ (OCH₂CH₂)₁₆₋₂₀—*** where *** is a point of attachment to TPL.

83. The compound of any one of claims 1-74, wherein L is —(C₂₋₂₀ alkylene)-(OCH₂CH₂)₂₋₄—(C₀₋₄ alkylene)-***, —(C₂₋₂₀ alkylene)-(OCH₂CH₂)₅₋₇—(C₀₋₄ alkylene)-***, —(C₂₋₂₀ alkylene)-(OCH₂CH₂)₈₋₁₀—(C₀₋₄ alkylene)-***, —(C₂₋₂₀ alkylene)-(OCH₂CH₂)₁₁₋₁₃—(C₀₋₄ alkylene)-***, —(C₂₋₂₀ alkylene)-(OCH₂CH₂)₁₄₋₁₆—(C₀₋₄ alkylene)-***, —(C₂₋₂₀ alkylene)-(OCH₂CH₂)₁₇₋₂₀—(C₀₋₄ alkylene)-***, —(C₁₋₂₀ alkylene)-(OCH₂CH₂)₁₋₁₀—(C₀₋₄ alkylene)-C(O)—***, or —(C₁₋₂₀ alkylene)-(OCH₂CH₂)₁₁₋₂₀—(C₀₋₄ alkylene)-C(O)—***, where *** is a point of attachment to TPL.

84. The compound of any one of claims 1-74, wherein L is —O (CH₂CH₂O)₂₋₄—(C₀₋₄ alkylene)-***, —O (CH₂CH₂O)₅₋₇—(C₀₋₄ alkylene)-***, —O (CH₂CH₂O)₈₋₁₀—(C₀₋₄ alkylene)-***, —O (CH₂CH₂O)₁₁₋₁₃—(C₀₋₄ alkylene)-***, —O (CH₂CH₂O)₁₄₋₁₆—(C₀₋₄ alkylene)-***, —O (CH₂CH₂O)₁₆₋₂₀—(C₀₋₄ alkylene)-***, —O (CH₂CH₂O)₂₋₁₀—(C₀₋₄ alkylene)C(O)—***, or —O (CH₂CH₂O)₁₁₋₂₀—(C₀₋₄ alkylene)C(O)—***, where *** is a point of attachment to TPL.

85. The compound of any one of claims 1-74, wherein L is —(C₀₋₂₀ alkylene)-(OCH₂CH₂)₁₋₁₀ —(N(C₁₋₄ alkyl))-***, —(C₀₋₂₀ alkylene)-(OCH₂CH₂)₁₁₋₂₀—(N(C₁₋₄ alkyl))-***, —(C₀₋₂₀ alkylene)-(CH₂CH₂O)₁₋₁₀—(C₂₋₁₀ alkylene)-(N(C₁₋₄ alkyl))-(C₀₋₁₀ alkylene)-***, or —(C₀₋₂₀ alkylene)-(CH₂CH₂O)₁₁₋₂₀—(C₂₋₁₀ alkylene)-(N(C₁₋₄ alkyl))-(C₀₋₁₀ alkylene)-***, where *** is a point of attachment to TPL.

86. The compound of any one of claims 1-74, wherein L is —N(H)—(C₂₋₉ alkylene)-O—(C₁₋₆ alkylene)-C(O)—***, —N(H)—(C₁₀₋₂₀ alkylene)-O—(C₁₋₆ alkylene)-C(O)—***, —N(H)—[(C₂₋₄ alkylene)-O-]₂₋₆-(C₁₋₆ alkylene)-C(O)—***, —N(H)—[(C₂₋₄ alkylene)-O-]₇₋₁₅-(C₁₋₆ alkylene)-C(O)—***, —N(H)—(C₁₋₆ alkylene)-C(O)—***, —N(H)—(C₇₋₁₅ alkylene)-C(O)—***, —N(H)—[(C₂₋₄ alkylene)-O-]₂₋₆-(C₁₋₆ alkylene)-***, —N(H)—[(C₂₋₄ alkylene)-O-]₇₋₁₅-(C₁₋₆ alkylene)-***, —N(H)—(C₂₋₉ alkylene)-O—(C₁₋₆ alkylene)-C(O)N(C₁₋₆ alkyl)-(C₁₋₆ alkylene)-***, —N(H)—(C2-9 alkylene)-O—(C₁₋₆ alkylene)-C(O)N(H)—(C₁₋₆ alkylene)-***, —N(H)—[(C₂₋₄ alkylene)-O-]₂₋₆-(C₁₋₆ alkylene)-N(H)—(C₁₋₆ alkylene)-***, —N(H)—[(C₂₋₄ alkylene)-O-]₇₋₁₅-(C₁₋₆ alkylene)-N(H)—(C₁₋₆ alkylene)-***, —N(H)—[(C₂₋₄ alkylene)-O-]₂₋₆-(C₁₋₆ alkylene)-N(C₁₋₆ alkyl)-(C₁₋₆ alkylene)-***, or —N(H)—[(C₂₋₄ alkylene)-O-]₇₋₁₅-(C₁₋₆ alkylene)-N(C₁₋₆ alkyl)-(C₁₋₆ alkylene)-***, where *** is a point of attachment to TPL.

87. The compound of any one of claims 1-74, wherein L is —N(H)—(C₂₋₉ alkylene)-O—(C₁₋₆ alkylene)-C(O)—***, —N(H)—(C₁₀₋₂₀ alkylene)-O—(C₁₋₆ alkylene)-C(O)—***, —N(H)—[CH₂CH₂—O-]₂₋₆-(C₁₋₆ alkylene)-C(O)—***, —N(H)—[CH₂CH₂—O-]₇₋₁₅-(C₁₋₆ alkylene)-C(O)—***, —N(H)—(C₁₋₆ alkylene)-C(O)—***, —N(H)—(C₇₋₁₅ alkylene)-C(O)—***, —N(H)—[CH₂CH₂—O-]₂₋₆-(C₁₋₆ alkylene)-***, —N(H)—[CH₂CH₂—O-]₇₋₁₅-(C₁₋₆ alkylene)-***, —N(H)—(C₂₋₉ alkylene)-O—(C₁₋₆ alkylene)-C(O)N(C₁₋₆ alkyl)-(C₁₋₆ alkylene)-***, —N(H)—(C₂₋₉ alkylene)-O—(C₁₋₆ alkylene)-C(O)N(H)—(C₁₋₆ alkylene)-***, —N(H)—[CH₂CH₂—O-]₂₋₆-(C₁₋₆ alkylene)-N(H)—(C₁₋₆ alkylene)-***, —N(H)—[CH₂CH₂—O-]₇₋₁₅-(C₁₋₆ alkylene)-N(H)—(C₁₋₆ alkylene)-***, —N(H)—[CH₂CH₂—O-]₂₋₆-(C₁₋₆ alkylene)-N(C₁₋₆ alkyl)-(C₁₋₆ alkylene)-***, or —N(H)—[CH₂CH₂—O-]₇₋₁₅-(C₁₋₆ alkylene)-N(C₁₋₆ alkyl)-(C₁₋₆ alkylene)-***, where *** is a point of attachment to TPL.

88. The compound of any one of claims 1-74, wherein L is —N(H)—[(C₂₋₄ alkylene)-O-]₂₋₆-(C₁₋₆ alkylene)-C(O)—***, —N(H)—[(C₂₋₄ alkylene)-O-]₇₋₁₅-(C₁₋₆ alkylene)-C(O)—***, —N(H)—(C₁₋₆ alkylene)-N(C₁₋₆ alkyl)C(O)—(C₁₋₆ alkylene) ***, —N(H)—(C₁₋₆ alkylene)-N(H)C(O)—(C₁₋₆ alkylene) ***, —N(H)—(C₂₋₆ alkylene)-***, —N(H)—(C₇₋₁₅ alkylene)-***, —N(C₁₋₆ alkyl)-(C₂₋₆ alkylene)-***, —N(C₁₋₆ alkyl)-(C₇₋₁₅ alkylene)-***, —N(H)—[(C₂₋₄ alkylene)-O-]₂₋₆-(C₁₋₆ alkylene)-***, —N(H)—[(C₂₋₄ alkylene)-O-]₇₋₁₅-(C₁₋₆ alkylene)-***, —N(H)—(C₁₋₆ alkylene)-(3-6 membered heterocycloalkylene)-(C₁₋₆ alkylene)-N(C₁₋₆ alkyl)-(C₁₋₆ alkylene)-***, —N(H)—(C₁₋₆ alkylene)-(3-6 membered heterocycloalkylene)-(C₁₋₆ alkylene)-N(H)—(C₁₋₆ alkylene)-***, —N(H)—(C₂₋₆ alkylene)-N(H)—(C₁₋₆ alkylene)-***, or —N(H)—(C₂₋₆ alkylene)-N(C₁₋₆ alkyl)-(C₁₋₆ alkylene)-***, where *** is a point of attachment to TPL.

89. The compound of any one of claims 1-74, wherein L is —N(H)—[CH₂CH₂—O-]₂₋₆-(C₁₋₆ alkylene)-C(O)—***, —N(H)—[CH₂CH₂—O-]₇₋₁₅-(C₁₋₆ alkylene)-C(O)—***, —N(H)—(C₁₋₆ alkylene)-N(C₁₋₆ alkyl)C(O)—(C₁₋₆ alkylene) ***, —N(H)—(C₁₋₆ alkylene)-N(H)C(O)—(C₁₋₆ alkylene) ***, —N(H)—(C₂₋₆ alkylene)-***, —N(H)—(C₇₋₁₅ alkylene)-***, —N(C₁₋₆ alkyl)-(C₂₋₆ alkylene)-***, —N(C₁₋₆ alkyl)-(C₇₋₁₅ alkylene)-***, —N(H)—[CH₂CH₂—O-]₂₋₆-(C₁₋₆ alkylene)-***, —N(H)—[CH₂CH₂—O-]₇₋₁₅-(C₁₋₆ alkylene)-***, —N(H)—(C₁₋₆ alkylene)-(3-6 membered heterocycloalkylene)-(C₁₋₆ alkylene)-N(C₁₋₆ alkyl)-(C₁₋₆ alkylene)-***, —N(H)—(C₁₋₆ alkylene)-(3-6 membered heterocycloalkylene)-(C₁₋₆ alkylene)-N(H)—(C₁₋₆ alkylene)-***, —N(H)—(C₂₋₆ alkylene)-N(H)—(C₁₋₆ alkylene)-***, or —N(H)—(C₂₋₆ alkylene)-N(C₁₋₆ alkyl)-(C₁₋₆ alkylene)-***, where *** is a point of attachment to TPL.

90. The compound of any one of claims 1-74, wherein L is —[(C₂₋₄ alkylene)-O-]₂₋₆-(C₁₋₆ alkylene)-***, —[(C₂₋₄ alkylene)-O-]₇₋₁₅-(C₁₋₆ alkylene)-***, —[(C₂₋₄ alkylene)-O-]₂₋₆-(C₁₋₆ alkylene)-N(C₁₋₆ alkyl)(C₁₋₆ alkylene)-***, —[(C₂₋₄ alkylene)-O-]₇₋₁₅-(C₁₋₆ alkylene)-N(C₁₋₆ alkyl)(C₁₋₆ alkylene)-***, —[(C₂₋₄ alkylene)-O-]₂₋₆-(C₁₋₆ alkylene)-N(H)(C₁₋₆ alkylene)-***, —[(C₂₋₄ alkylene)-O-]₇₋₁₅-(C₁₋₆ alkylene)-N(H)(C₁₋₆ alkylene)-***, —(C₁₋₉ alkylene)-C(O)N(H)—(C₁₋₆ alkylene)-***, —(C₁₋₉ alkylene)-N(H)C(O)—(C₁₋₆ alkylene)-***, —(C₁₋₉ alkylene)-C(O)N(H)—[(C₂₋₄ alkylene)-O-]₂₋₆-(C₁₋₆ alkylene)-***, —(C₁₋₉ alkylene)-N(H)C(O)—[(C₂₋₄ alkylene)-O-]₂₋₆-(C₁₋₆ alkylene)-***, —(C₁₋₉ alkylene)-C(O)N(H)—[(C₂₋₄ alkylene)-O-]₇₋₁₅-(C1-6 alkylene)-***, —(C₁₋₉ alkylene)-N(H)C(O)—[(C₂₋₄ alkylene)-O-]₇₋₁₅-(C₁₋₆ alkylene)-***, -(C₁₋₉ alkylene)-C(O)N(H)—[(C₂₋₄ alkylene)-O-]₂₋₆-(C₁₋₆ alkylene)-N(C₁₋₆ alkyl)-(C₁₋₆ alkylene)-***, —(C₁₋₉ alkylene)-N(H)C(O)—[(C₂₋₄ alkylene)-O-]₂₋₆-(C₁₋₆ alkylene)-N(C₁₋₆ alkyl)-(C₁₋₆ alkylene)-***, —(C₁₋₉ alkylene)-C(O)N(H)—[(C₂₋₄ alkylene)-O-]₇₋₁₅-(C₁₋₆ alkylene)-N(C₁₋₆ alkyl)-(C₁₋₆ alkylene)-***, or —(C₁₋₉ alkylene)-N(H)C(O)—[(C₂₋₄ alkylene)-O-]₇₋₁₅-(C₁₋₆ alkylene)-N(C₁₋₆ alkyl)-(C₁₋₆ alkylene)-***, where *** is a point of attachment to TPL.

91. The compound of any one of claims 1-74, wherein L is —[CH₂CH₂—O-]₂₋₆-(C₁₋₆ alkylene)-***, —[CH₂CH₂—O-]₇₋₁₅-(C₁₋₆ alkylene)-***, —[CH₂CH₂—O-]₂₋₆-(C₁₋₆ alkylene)-N(C₁₋₆ alkyl)(C₁₋₆ alkylene)-***, —[CH₂CH₂—O-]₇₋₁₅-(C₁₋₆ alkylene)-N(C₁₋₆ alkyl)(C₁₋₆ alkylene)-***, —[CH₂CH₂—O-]₂₋₆-(C₁₋₆ alkylene)-N(H)(C₁₋₆ alkylene)-***, —[CH₂CH₂—O-]₇₋₁₅-(C₁₋₆ alkylene)-N(H)(C₁₋₆ alkylene)-***, —(C₁₋₉ alkylene)-C(O)N(H)—(C₁₋₆ alkylene)-***, —(C₁₋₉ alkylene)-N(H)C(O)—(C₁₋₆ alkylene)-***, —(C₁₋₉ alkylene)-C(O)N(H)—[CH₂CH₂—O-]₂₋₆-(C₁₋₆ alkylene)-***, —(C₁₋₉ alkylene)-N(H)C(O)—[CH₂CH₂—O-]₂₋₆-(C₁₋₆ alkylene)-***, —(C₁₋₉ alkylene)-C(O)N(H)—[CH₂CH₂—O-]₇₋₁₅-(C₁₋₆ alkylene)-***, —(C₁₋₉ alkylene)-N(H)C(O)—[CH₂CH₂—O-]₇₋₁₅-(C₁₋₆ alkylene)-***, —(C₁₋₉ alkylene)-C(O)N(H)—[CH₂CH₂—O-]₂₋₆-(C₁₋₆ alkylene)-N(C₁₋₆ alkyl)-(C₁₋₆ alkylene)-***, —(C₁₋₉ alkylene)-N(H)C(O)—[CH₂CH₂—O-]₂₋₆-(C₁₋₆ alkylene)-N(C₁₋₆ alkyl)-(C₁₋₆ alkylene)-***, —(C₁₋₉ alkylene)-C(O)N(H)—[CH₂CH₂—O-]₇₋₁₅ —(C₁₋₆ alkylene)-N(C₁₋₆ alkyl)-(C₁₋₆ alkylene)-***, or —(C₁₋₉ alkylene)-N(H)C(O)—[(CH₂CH₂—O-]₇₋₁₅-(C₁₋₆ alkylene)-N(C₁₋₆ alkyl)-(C₁₋₆ alkylene)-***, where *** is a point of attachment to TPL.

92. The compound of any one of claims 1-74, wherein L is —N(H)—[(C₂₋₄ alkylene)-O-]₂₋₆-(C₁₋₆ alkylene)-N(H)—***, —N(H)—[(C₂₋₄ alkylene)-O-]₇₋₁₅-(C₁₋₆ alkylene)-N(H)—***, —N(C₁₋₆ alkyl)-[(C₂₋₄ alkylene)-O-]₂₋₆-(C₁₋₆ alkylene)-N(H)—***, —N(C₁₋₆ alkyl)-[(C₂₋₄ alkylene)-O-]₇₋₁₅ —(C₁₋₆ alkylene)-N(H)—***, —N(C₁₋₆ alkyl)-[(C₂₋₄ alkylene)-O-]₂₋₆-(C₁₋₆ alkylene)-N(C₁₋₆ alkyl)-***, or —N(C₁₋₆ alkyl)-[(C₂₋₄ alkylene)-O-]₇₋₁₅-(C₁₋₆ alkylene)-N(C₁₋₆ alkyl)-***, where *** is a point of attachment to TPL.

93. The compound of any one of claims 1-74, wherein L is —N(H)—[CH₂CH₂—O-]₂₋₆-(C₁₋₆ alkylene)-N(H)—***, —N(H)—[CH₂CH₂—O-]₇₋₁₅-(C₁₋₆ alkylene)-N(H)—***, —N(C₁₋₆ alkyl)-[CH₂CH₂—O-]₂₋₆-(C₁₋₆ alkylene)-N(H)—***, —N(C₁₋₆ alkyl)-[CH₂CH₂—O-]₇₋₁₅-(C₁₋₆ alkylene)-N(H)—***, —N(C₁₋₆ alkyl)-[CH₂CH₂—O-]₂₋₆-(C₁₋₆ alkylene)-N(C₁₋₆ alkyl)-***, or —N(C₁₋₆ alkyl)-[CH₂CH₂—O-]₇₋₁₅-(C₁₋₆ alkylene)-N(C₁₋₆ alkyl)-***, where *** is a point of attachment to TPL.

94. The compound of any one of claims 1-74, wherein L is —(C₂₋₁₀ alkylene)-(OCH₂CH₂)₂₋₄—O-(3-6 membered saturated heterocyclylene containing 1 or 2 heteroatoms independently selected from nitrogen and oxygen)-(C₁₋₅ alkylene)-***, —(C₂₋₁₀ alkylene)-(3-6 membered saturated heterocyclylene containing 1 or 2 heteroatoms independently selected from nitrogen and oxygen)-(C₁₋₅ alkylene)-***, —(C₂₋₁₀ alkylene)-N(H)—(C₁₋₅ alkylene)-***, —(C₂₋₁₀ alkylene)-N(C₁₋₆ alkyl)-(C₁₋₅ alkylene)-***, —N(H)—(C₁₋₅ alkylene)-***, —(CH₂CH₂O)₁₋₄—(C₁₋₄ alkylene)-***, —(CH₂CH₂O)₁₋₄—(C₁₋₄ alkylene)-N(C₁₋₆ alkyl)-(C₁₋₆ alkylene)-***, —N(H)—(C₂₋₆ alkylene)-(3-6 membered saturated heterocyclylene containing 1 or 2 heteroatoms independently selected from nitrogen and oxygen)-(C₁₋₅ alkylene)-***, —N(H)—(C₂₋₆ alkylene)-(OCH₂CH₂)₁₋₄—O-(3-6 membered saturated heterocyclylene containing 1 or 2 heteroatoms independently selected from nitrogen and oxygen)-(C₁₋₅ alkylene)-***, —N(H)—[—CH₂CH₂O-]₂₋₆-(C₁₋₆ alkylene)-N(C₁₋₆ alkyl)-(C₁₋₆ alkylene)-***, —(C₁₋₆ alkylene)-(3-6 membered saturated heterocyclylene containing 1 or 2 heteroatoms independently selected from nitrogen and oxygen)-(C₁₋₆ alkylene)-***, —N(H)—(C₂₋₁₀ alkylene)-***, —(C₁₋₆ alkylene)-(3-6 membered saturated heterocyclylene containing 1 or 2 heteroatoms independently selected from nitrogen and oxygen)-(C₁₋₆ alkylene)-N(C₁₋₆ alkyl)-(C₁₋₆ alkylene)-***, —N(H)—[CH₂CH₂—O-]₂₋₆-(C₁₋₆ alkylene)-N(C₁₋₆ alkyl)-(C₁₋₆ alkylene)-***, —N(H)—[CH₂CH₂—O-]₂₋₁₀-(C₁₋₆ alkylene)-***, —N(H)—[CH₂CH₂—O-]₂₋₆-(C₁₋₆ alkylene)-N(C₁₋₆ alkyl)C(O)—(C₁₋₆ alkylene)-***, —[CH₂CH₂—O-]₁₋₆-(C₁₋₆ alkylene)-N(C₁₋₆ alkyl)-***, —[CH₂CH₂—O-]₁₋₆-(C₁₋₆ alkylene)-N(H)—***, or —(C₂₋₁₀ alkylene)-(OCH₂CH₂)₂₋₆—(C₃₋₆ cycloalkylene)-***, where *** is a point of attachment to TPL.

95. A compound in Table 1 or 2, or a pharmaceutically acceptable salt thereof.

96. A compound in Table 1-A or 2-A, or a pharmaceutically acceptable salt thereof.

97. A pharmaceutical composition comprising a compound of any one of claims 1-96 and a pharmaceutically acceptable carrier.

98. A method of treating cancer, comprising administering to a patient in need thereof a therapeutically effective amount of a compound of any one of claims 1-96 to treat the cancer.

99. The method of claim 98, wherein the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct cancer, gallbladder cancer, liver cancer, pancreatic cancer, bladder cancer, urinary tract cancer, kidney cancer, eye cancer, thyroid cancer, lymphoma, or leukemia.

100. A method of causing death of a cancer cell, comprising contacting a cancer cell with an effective amount of a compound of any one of claims 1-96 to cause death of the cancer cell.

101. The method of claim 100, wherein the cancer cell is selected from an ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct cancer, gallbladder cancer, liver cancer, pancreatic cancer, bladder cancer, urinary tract cancer, kidney cancer, eye cancer, thyroid cancer, lymphoma, or leukemia cell.
